(19) United States
(12) Patent Application Publication
(10) Pub. No.: US 2024/0401050 A1
(43) Pub. Date: Dec. 5, 2024

(54) Nucleic Acid Sponges

(71) Applicant: National University of Singapore, Singapore (SG)

(72) Inventors: Annadoray LAVENNIAH, Singapore (SG); Volker PATZEL, Singapore (SG); Roger SY FOO, Singapore (SG)

(21) Appl. No.: 18/755,790

(22) Filed: Jun. 27, 2024

Related U.S. Application Data
(63) Continuation of application No. PCT/SG2022/050939, filed on Dec. 28, 2022.
(60) Provisional application No. 63/295,755, filed on Dec. 31, 2021.

(51) Int. Cl. C12N 15/113

(52) U.S. Cl. CPC C12N 15/113; C12N 2310/113; C12N 2310/333; C12N 2310/3341; C12N 2310/335; C12N 2310/531

(57) ABSTRACT

The disclosure herein relates to engineered nucleic acids. These engineered nucleic acids are adapted to bind to RNA molecules such as microRNA and by doing so can affect RNA post transcriptional modification and RNA translation. Kits, pharmaceutical compositions, and methods of treatment using the engineered nucleic acids are also disclosed.

### CROSS-REFERENCE

This application is a continuation of PCT/SG2022/050939, filed Dec. 28, 2022, which claims the benefit of U.S. Provisional Application No. 63/295,755 filed Dec. 31, 2021, the disclosures of which are incorporated herein by reference in their entirety.

### SEQUENCE LISTING

The instant application contains a Sequence Listing which has been submitted electronically in ST.26 (xml) format and is hereby incorporated by reference in its entirety. Said ST.26 (xml) copy, created on Jun. 12, 2024, is named 203777_701301_SL.xml and is 687,274 bytes in size.

### SUMMARY

Disclosed herein are engineered nucleic acids comprising a plurality of stem loops wherein each stem loop of the plurality is joined to at least one additional stem loop of the plurality of stem loops by a linker sequence, wherein each loop of each stem loop of the plurality of stem loops independently comprises a binding site that is independently configured to hybridize to a target RNA, and wherein the engineered nucleic is not circularized. In some embodiments, an engineered nucleic acid can comprise from about 50 to about 500, or to about 550 nucleotides. In some embodiments, the plurality of stem loops can comprise 3, 4, 5, 6, 7, or 8 stem loops. In some embodiments, each stem loop of the plurality of stem loops can independently comprise a stem comprising from about 5 to about 20 base pairs or about 12 to about 15 base pairs. In some embodiments, the stem can comprise a deletion in one arm of the stem, a mismatch in the stem, or both. In some embodiments, each stem of each stem loop of the plurality of stem loops can comprise a substantially complimentary number of nucleotide base pairs. In some embodiments, each linker sequence can be independently from about 2 to about 12 nucleotides in length or about 6 to 8 nucleotides in length. In some embodiments, each loop of each stem loop of the plurality of stem loops independently can comprise from about 15 to about 75 nucleotides. In some embodiments, each binding site of each loop of the plurality of stem loops, when hybridized to the target RNA, independently can comprise a mismatched base with respect to a corresponding base of the target RNA. In some embodiments, each binding site of each loop of the plurality of stem loops, when independently hybridized to an individual target RNA, individually can comprise a second mismatched base with respect to a second corresponding base of the target RNA. In some embodiments, the corresponding base of the target RNA can be positioned within a first 8 nucleotides of a 5′ end of the target RNA. In some embodiments, each stem loop of the plurality of stem loops can be the same. In some embodiments, at least one stem loop of the plurality of stem loops can differ from the remaining stem loops of the plurality of stem loops. In some embodiments, a binding site of a loop of a stem loop of the engineered nucleic acid can be configured upon the binding or hybridizing to a target RNA to produce a mismatch between a base of the binding site of a loop of a stem loop and a base of the target RNA. In some embodiments, a binding site of a loop of a stem loop of the engineered nucleic acid can be configured upon the binding or hybridizing to a target RNA to produce a second mismatch between a second base of the binding site of the loop of the stem loop and a second base of the target RNA. In some embodiments, at least one mismatch is an A/C mismatch where the A can be in the binding site of the loop of the stem loop and the C can be in the target RNA or the A can be in the target RNA and the C can be in the binding site of the loop of the stem loop. In some embodiments, at least one mismatch is an A/G mismatch where the A can be in the binding site of the loop of the stem loop and the G can be in the target RNA or the A can be in the target RNA and the G can be in the binding site of the loop of the stem loop. In some embodiments, at least one mismatch is a T/G mismatch where the T can be in the binding site of the loop of the stem loop and the G can be in the target RNA or the T can be in the target RNA and the G can be in the binding site of the loop of the stem loop. In some embodiments, at least one mismatch is a T/C mismatch where the T can be in the binding site of the loop of the stem loop and the C can be in the target RNA or the T can be in the target RNA and the C can be in the binding site of the loop of the stem loop. In some embodiments, at least one mismatch is a U/G mismatch where the U can be in the binding site of the loop of the stem loop and the G can be in the target RNA or the U can be in the target RNA and the G can be in the binding site of the loop of the stem loop, and optionally wherein the U/G mismatch is a wobble base pair. In some embodiments, at least one mismatch a U/C mismatch where the U can be in the binding site of the loop of the stem loop and the C can be in the target RNA or the U can be in the target RNA and the C can be in the binding site of the loop of the stem loop. In some embodiments, a base of a mismatch can comprise a C, an A, a G, a T, or a U. In some embodiments, a base of each mismatch can independently comprise a C, an A, a G, a T, or a U. In some embodiments, the loop of a stem loop can comprise a first binding site and a second binding site. In some embodiments, the first binding site and the second binding site are the same. In some embodiments, the first binding site and the second binding site are different. In some embodiments, each loop of each stem loop of the plurality of stem loops can be configured to hybridize to the same target RNA. In some embodiments, each loop of the plurality of stem loops can be configured to hybridize a different target RNA than at least one other binding site of another loop of a stem loop of the plurality of stem loops. In some embodiments, each stem loop of the plurality of stem loops can be substantially radially disposed at a substantially regular distance around a partial loop comprising linkers. In some embodiments, the engineered nucleic acid when depicted in two dimensions, in a geometric confirmation substantially can display a C3 C4, C5, C6, C7, or C8 symmetries. In some embodiments, the engineered nucleic acid can further comprise a nuclear localization sequence. In some embodiments, the engineered nucleic acid can comprise a modified nucleotide. In some embodiments, the modified nucleotide can comprise pseudouridine, 5-methylcytidine, n1-methylpseudouridine, N6-methyladenosine, or any combination thereof. In some embodiments, the engineered nucleic acid can further comprise a chemical modification. In some embodiments, the chemical modification can comprise a phosphodiester modification, methylation of a hydroxyl group, an epigenetically marked base, a deoxyribose sugar, or a combination thereof. In some embodiments, the chemical modification comprises the phosphodiester modification. In some embodiments, the chemical modification comprises the methylation of a hydroxyl group. In some embodiments, the chemical modification comprises the epigenetically marked base. In some embodiments, the chemical modification comprises the deoxyribose sugar. In some embodiments, the target RNA can be a messenger RNA (mRNA), a ribosomal RNA (rRNA), a transfer RNA (tRNA), a ribozyme a transfer messenger RNA (tmRNA), a double stranded RNA (dsRNA), a small nuclear RNA (ssRNA), a small nucleolar RNA (snoRNA), a Piwi-interacting RNAs (piRNA), a microRNA (miRNA), a small interfering RNA (siRNA), a long non-coding RNA (lncRNA), or any combination thereof. In some embodiments, the target RNA can comprise the miRNA. In some embodiments, the binding site of a first loop of the plurality of stem loops can be configured to bind or hybridise to a second binding site of a second stem loop of the plurality of stem loops. In some embodiments, the nucleic acid comprises RNA. In some embodiments, the engineered nucleic acid can comprise at least about 80%, 85%, 90%, 95%, 96%, 97%, 98%, or 99% sequence identity to any one of SEQ ID NOs: 253-330. In some embodiments, the engineered nucleic acid can comprise at least about 80%, 85%, 90%, 95%, 96%, 97%, 98%, or 99% sequence identity to any one of SEQ ID NOs: 47-78 or 427-432. In some embodiments, the engineered nucleic acid can comprise at least about 80%, 85%, 90%, 95%, 96%, 97%, 98%, or 99% sequence identity to any one of SEQ ID Nos 433-511 or 512-583. In some embodiments, the engineered nucleic acid can comprise at least about 80%, 85%, 90%, 95%, 96%, 97%, 98%, or 99% sequence identity to any one of SEQ ID NOs: 331-426. In some embodiments, the engineered nucleic acid can comprise at least about 80%, 85%, 90%, 95%, 96%, 97%, 98%, or 99% sequence identity to SEQ ID NO: 435, 438, 514, or 517. In some embodiments, the engineered nucleic acid comprises at least about 80%, 85%, 90%, 95%, 96%, 97%, 98%, or 99% sequence identity to SEQ ID NO: 441, 449, 506, 521, 528, or 578. In some embodiments, the nucleic acid can comprise DNA. In some embodiments, the engineered nucleic acid can comprise at least about 80%, 85%, 90%, 95%, 96%, 97%, 98%, or 99% sequence identity to any one of SEQ ID NOs: 79-174. In some embodiments, the engineered nucleic acid can comprise at least about 80%, 85%, 90%, 95%, 96%, 97%, 98%, or 99% sequence identity to any one of SEQ ID NOs: 175-252.

Also disclosed herein are DNAs encoding the engineered nucleic acid described above.

Also disclosed herein are vectors containing or encoding engineered nucleic acids described above or the DNAs described above. In some embodiments, the vector can comprise a viral vector, a nanoparticle vector, a liposomal vector, or a combination thereof. In some embodiments, the vector can comprise the viral vector. In some embodiments, the viral vector can comprise a DNA viral vector. In some embodiments, the DNA viral vector comprises an adeno associated virus vector. In some embodiments, the adeno associated virus vector can comprise an AAV 1, AAV 2 AAV 3, AAV 4, AAV 5, AAV 6, AAV 7, AAV 8, AAV 9 or any combination thereof. In some embodiments, the DNA viral vector can comprise an adenoviral vector. In some embodiments, the viral vector can comprise a retroviral vector. In some embodiments, the retroviral vector can comprise a nuclear localization signal. In some embodiments, the vector can comprise the liposomal vector. In some embodiments, the liposomal vector can comprise RNA. In some embodiments, the liposomal vector can comprise DNA. In some embodiments, the liposomal vector can comprise a plasmid. In some embodiments, the vector can comprise a nanoparticle vector.

Also disclosed herein are pharmaceutical compositions comprising the engineered nucleic acids, the DNAs, or the vectors described above and a pharmaceutically acceptable: excipient, diluent, or carrier. In some embodiments, the excipient can comprise a buffering agent, a stabilizer, an antioxidant, a binder, a diluent, a dispersing agent, a rate controlling agent, a lubricant, a glidant, a disintegrant, a plasticizer, a preservative, or any combinations thereof. In some embodiments, the diluent can comprise distilled water, physiological saline, Ringer's solutions, dextrose solution, a cell growth medium, phosphate buffered saline (PBS), or any combination thereof. In some embodiments, the pharmaceutical composition can be in unit dose form.

Also disclosed herein are kits comprising: the engineered nucleic acids, the DNAs, the vectors, or the pharmaceutical compositions described above and a container.

Also disclosed herein are methods of treating or preventing a disease or condition in a subject. In some embodiments, the method can comprise administering to the subject a therapeutically effective amount of the engineered nucleic acids, the DNAs, the vectors, or the pharmaceutical compositions described above. In some embodiments, the subject is in need thereof. In some embodiments, the subject is a human subject. In some embodiments, the administering is in an amount of from about 0.001 mg to about 100 mg of the engineered nucleic acid, the DNA, the vector, or the pharmaceutical composition per kg of body weight of the subject. In some embodiments, the administering can be performed at least once in a 24-hour time period. In some embodiments, the administering can be intramuscular, intravenous, intraocular, intraperitoneal, intracardial, subcutaneous, intracranial, intrathecal, oral, inhalation, or any combination thereof. In some embodiments, the disease or condition can be a cardiac disease, a neurological disease, cancer, a viral disease or a fungal disease. In some embodiments, the disease or condition comprises the cardiac disease that comprises hypertension, a metabolic syndrome, a valve disease, cardiac hypertrophy, cardiac hypotrophy, cardiac fibrotic remodeling, cardiac wall stiffness, stable angina, unstable angina, variant angina, atrial fibrillation, hart block, premature atrial complex, atrial flutter, paroxysmal supraventricular tachycardia, Wolff-Parkinson-White syndrome, premature ventricular complex, ventricular tachycardia, ventricular fibrillation, long QT syndrome, dilated cardiomyopathy, hypertrophic cardiomyopathy, restrictive cardiomyopathy, congestive heart failure, arterial septal defect, ventricular septal defect, patent ductus arteriosis, pulmonic stenosis, congenital aortic stenosis, coarctation of aorta, tetraology of Fallot, tricuspid atresia, truncus arteriosus, Ebstein's anomaly of the tricuspid valve, cor pulmonale, myocardial infarction, mitral stenosis, mitral valve regurgitation, mitral valve prolapse, aortic stenosis, aortic regurgitation, tricuspid stenosis, tricuspid regurgitation, myocarditis, pericarditis, rheumatic heart disease, cardiac tumor, aortic aneurysm, arteriosclerosis, atherosclerosis, aortic dissection, hypertension, transient ischemic attack, other cardiac related diseases, or a combination thereof. In some embodiments, the disease or condition can comprise a cancer that comprises at least one of: a melanoma, a hepatocellular carcinoma, a breast cancer, a lung cancer, a non-small lung cancer, a peritoneal cancer, a prostate cancer, a bladder cancer, an ovarian cancer, a leukemia, a lymphoma, a renal cell carcinoma, a pancreatic cancer, an epithelial carcinoma, a gastric/GE junction adenocarcinoma, a cervical cancer, a colon carcinoma, a colorectal cancer, a duodenal cancer, a pancreatic adenocarcinoma, an adenoid cystic, a sarcoma, a mesothelioma, a glioblastoma multiforme, a astrocytoma, a multiple myeloma, a prostate carcinoma, a hepatocellular carcinoma, a cholangiocarcinoma, a pancreatic adenocarcinoma, a head and neck squamous cell carcinoma, a cervical squamous-cell carcinoma, an osteosarcoma, an epithelial ovarian carcinoma, an acute lymphoblastic lymphoma, a myeloproliferative neoplasm, any other malignant condition or any variant thereof. In some embodiments, the disease or condition can comprise the viral disease that comprises at least one of: a coronavirus disease, an influenza disease, a parainfluenza virus disease, a herpesvirus disease, an adenovirus disease, a flavivirus disease, a retroviral disease, a paramyxovirus based disease, a parvovirus based disease, or a combination thereof. In some embodiments, the disease or condition can comprise a fungal disease that comprises at least one of: blastomycosis, coccidioidomycosis, cryptococcosis, histoplasmosis, paracoccidioidomycosis, aspergillosis, candidiasis, mucormycosis, talaromycosis or a combination thereof. In some embodiments, the method can further comprise concurrently or consecutively administering a second therapy. In some embodiments, the disease or condition can be the cardiac disease, and the second therapy is an ace inhibitor, an angiotensin 2 receptor antagonist, an antiarrhythmic, an anticoagulant, a platelet inhibitor, an antihypertensive, a beta blocker, a calcium channel blocker, digitoxin, a statin, nitroglycerin, or a combination thereof. In some embodiments, the disease or condition can be the viral disease and the second therapy is an antiviral drug, an anti-inflammatory, a cytokine storm inhibitor, or a combination thereof. In some embodiments, the pharmaceutical composition comprises a liquid dosage form that can be administered at a volume of about 1 ml to about 5 ml, about 5 ml to 10 ml, about 15 ml to about 20 ml, about 25 ml to about 30 ml, about 30 ml to about 50 ml, about 50 ml to about 100 ml, about 100 ml to 150 ml, about 150 ml to about 200 ml, about 200 ml to about 250 ml, about 250 ml to about 300 ml, about 300 ml to about 350 ml, about 350 ml to about 400 ml, about 400 ml to about 450 ml, about 450 ml to 500 ml, about 500 ml to 750 ml, or about 750 ml to 1000 ml. In some embodiments, the subject can receive the pharmaceutical composition at a dosing level from about 0.001 mg/kg to about 10,000 mg/kg, wherein mg/kg is mg engineered nucleic acid per kilogram of subject body weight. In some embodiments, the administering can be in a liquid dosage form, a solid dosage form, an inhalable dosage form, an intranasal dosage form, a liposomal formulation, or any combinations thereof. In some embodiments, the administration can comprise at least partially systemic administration. In some embodiments, the at least partially systemic administration can comprise at least one of: an oral administration, an intravenous administration, an intranasal administration, a sublingual administration, a rectal administration, a transdermal administration, an intradermal administration, an intraurethral administration, an intravaginal administration, an intrathecal administration, an intramuscular administration, an intraperitoneal administration, an intratumoral administration, or any combinations thereof.

Also disclosed herein are isolated cells comprising the engineered nucleic acids, the DNAs, or the vectors described above.

Also disclosed herein are methods of conducting an in vitro assay. In some embodiments, the method comprises conducting the in vitro assay employing the engineered nucleic acids, the DNAs, or the vectors described above. In some embodiments, the assay can comprise detection of a reporter gene. In some embodiments, the reporter gene can comprise luciferase, beta galactosidase, a fluorescent protein, chloramphenicol acetyltransferase, or a combination thereof. In some embodiments, the reporter gene can comprise luciferase. In some embodiments, the reporter gene can comprise beta galactosidase. In some embodiments, the reporter gene can comprise a fluorescent protein. In some embodiments, the reporter gene can comprise chloramphenicol acetyltransferase.

Also disclosed herein are methods of increasing expression of a polypeptide translated from an mRNA template that is a substrate for an miRNA. In some embodiments, the method can comprise administering to a cell that comprises the mRNA template and the miRNA an effective amount of the engineered nucleic acid described above. In some embodiments, the effective amount can comprise an amount of the engineered nucleic acid that is sufficient to hybridize to the miRNA in the cell, thereby increasing translation of the mRNA template, as compared to an amount of translation prior to the administering, as determined in an in vitro assay.

Also disclosed herein are methods of sequestering a plurality of target RNAs with an engineered nucleic acid described above comprising binding at least three loops of the plurality joined individually to at least one protein in a metabolic pathway. In some embodiments, the metabolic pathway can comprise a target RNA encoding a protein comprising PBP1a/b. PBP4, PBP2c, PBP2d, PBP2a, PbpH, PBP2b, PBP3, SpoVD, PBP4b, PBP5, PBP4a, DacF, PbpX, MepA, AmpC, AmpH or any combination thereof. In some embodiments, the metabolic pathway can comprise affecting the shape of a bacteria and the proteins can comprise PBP4, PBP5, PBP7, AmpC, AmpH, or any combination thereof.

### BRIEF DESCRIPTION OF THE DRAWINGS

FIGS. 1A-B depicts structures of paired and unpaired loops of stem loops each carrying one miR-155-5p binding site, wherein FIG. 1A depicts loop structures, which can include one or more stem loops, contained within a loop of a stem loop and a second stem loop and FIG. 1B depicts two stem loops. Base paired stem sequences are highlighted in black: miR-155-5p miRNA bulged binding sites, which may comprise one deletion and two mismatches at positions at positions 9-11 of a 5′ end of a target miRNA, are highlighted in light gray: 5′ and 3′ auxiliary arm regions flanking the miRNA binding site are in lower case letters. Dot bracket notation and graphical representations of RNA minimum free energy (MFE) and centroid secondary structures were predicted using the Vienna RNAfold web server. FIG. 1A discloses SEQ ID NOS 47, 47, 47, and FIG. 1B discloses SEQ ID NOS 427, 427, and 427, respectively, in order of appearance.

FIG. 2 is a depiction of different base paired stem sequences for each miRNA target site. Nucleotide sequence of 6 stem loops with different base pairing stem regions but same auxiliary arm regions (e.g., a part of a stem loop or loop that does not bind to a target RNA) for a single miR-155-5p binding site. Base paired stem sequences are highlighted in black: miR-155-5p miRNA bulged binding sites are highlighted in light gray: 5′ and 3′ auxiliary arm regions flanking the miRNA binding site are in lower case letters. Figure discloses SEQ ID NOS 427-432, respectively, in order of appearance.

FIGS. 3A-B is a depiction of linking individual stem loops to form a snowflake-like structure when viewed in a two-dimensional form wherein FIG. 3A depicts a 3 nt sequence comprising of Us were used to link 6 different base paired stem-loops and FIG. 3B depicts base pairing stem sequences that were edited to allow for unpaired loop formation within the snowflake-like structure when viewed in a two-dimensional form, when the engineered polynucleotides are depicted as snowflake like structures in two dimensions. Edited nucleotides are highlighted with black arrows. Base paired stem sequences are highlighted in black: miR-155-5p miRNA bulged binding sites are highlighted in light gray: 5′ and 3′ auxiliary arm regions flanking the miRNA binding site are in lower case letters. Dot bracket notation and graphical representations of RNA minimum free energy (MFE) and centroid secondary structures were predicted using the Vienna RNAfold web server. FIG. 3A discloses SEQ ID NOS 49-54, and FIG. 3B discloses SEQ ID NOS 49, 56, 51, 58, 53, and 60, respectively, in order of appearance.

FIGS. 4A-C depicts illustrations and sequences showing improved flexibility of ‘snowflake’-like structure when viewed in a two-dimensional form wherein FIG. 4A illustrates sequences with a single base deletion at the center of the base pairing stem sequence. Figure discloses SEQ ID NOS 61-66, respectively, in order of appearance; FIG. 4B illustrates 8 nt linkers regions with a 30% GC content. Figure discloses SEQ ID NOS 67-72, 67, and 74-78, respectively, in order of appearance; and FIG. 4C, which depicts engineered polynucleotides as snowflake like structures in two dimensions, illustrates different 5′ and 3′ auxiliary arm sequences that were used for each stem-loop. In FIGS. 4A-C, single base deletions are underscored. Base paired stem sequences are highlighted in black: miR-155-5p miRNA bulged binding sites are highlighted in light gray: 5′ and 3′ auxiliary arm regions flanking the miRNA binding site are in lower case letters. Dot bracket notation and graphical representations of RNA minimum free energy (MFE) and centroid secondary structures were predicted using the Vienna RNAfold web server.

FIGS. 5A-B depicts structural features of C/D box and H/ACA box snoRNAs, wherein FIG. 5A depicts a C/D box of two conserved sequence elements named box C (RUGAUGA), and box D (CUGA), and wherein FIG. 5B depicts an H/ACA box snoRNA containing evolutionarily conserved structural elements, including box H (ANANNA), box ACA motif, and two pseudouridinylation pockets. Antisense elements of FIG. 5A upstream of the D/D′ motifs are complementary to target RNAs and catalyze site specific 2′-O-methylation (2′-O-Me) of the NT target RNAs. FIG. 5B further illustrates that the pseudouridinylation pockets are complementary to the substrate RNAs and responsible for pseudouridinylation (NY′).

FIG. 6 depicts nucleotide positions at and surrounding the AGCCC motif responsible for nuclear localization.

FIG. 7 depicts sequence alignment of the four most effective tile regions associated with nuclear localization were derived from lncRNAs JPX, PVT1, NR2F1-AS1 and Emxos. The consensus sequence of the AluSx repeat family and the SIRLOIN element are shown. C/T-rich hexamers in bold text. Figure discloses SEQ ID NOS 588-593, respectively, in order of appearance.

FIG. 8 depicts an engineered RNA sequence in a secondary structural form showing a snowflake configuration when viewed two-dimensionally, comprising as viewed outward from a center point, linker sequences and a homologous loop comprising substantially complementary base pairs. Stems of stem loops are shown in bold (e.g., homology arms), outward facing auxiliary arms are shown in lowercase. The auxiliary arms are depicted as outward from between the binding site of a engineered nucleic acid. The binding site itself is outward from the auxiliary arms and is adapted to interact with a nucleic acid. Figure discloses SEQ ID NO: 594.

FIG. 9 depicts miR binding sites for miR-17-5p targets. The binding sites shown are 1) perfect seed, 2) imperfect seed, 3) imperfect bulge and perfect seed, and 4) imperfect bulge and imperfect seed. Figure discloses SEQ ID NOS 595, 79, 595-596, 595, 84, 595, and 597, respectively, in order of appearance.

FIG. 10 depicts engineered nucleic acid binding site design choices with perfect seeds, and imperfect seeds. Perfect seeds are those sequences which comprise perfect complementary between the 2-8 nt region from the 5′ end of the target binding site. An imperfect seed comprises inserting either an A, G, C, or U nucleotide in position 6 from the 3′ end of the target site.

FIG. 11 depicts engineered nucleic acids using RNA, as snowflake like forms in two dimensions, which were designed to sponge one of several miRNAs. The miRNAs include miR-17, miR-132, and miR-155. Linkers that can be used in the central ring are: 2 nt (nucleotide) linkers, 4 nt linkers, 6 nt linkers, and 8 nt linkers.

FIG. 12 depicts engineered nucleic acids using RNA, in snowflake like form in two dimensions, which were designed to sponge one of several miRNAs. The miRNAs include miR-17, miR-132, and miR-155. The length of the homology arms can be 10 nt or 15 nt (currently shown). The length of the auxiliary arms can be 8 nt, 11 nt or 12 nt. There can be 2, 4, 6 (currently shown), or 8 binding sites. FIG. 12 also depicts an engineered nucleic acid sequence in a secondary structural form comprising as viewed outward from a center point, linker sequences and a homologous loop comprising substantially complementary base pairs. Stems of stem loops (e.g., homology arms) are shown in black boxes, outward facing auxiliary arms are shown in white curves. The auxiliary arms are depicted as outward from between the binding site of a engineered nucleic acid. The binding site itself is outward from the auxiliary arms and is engineered to interact with a nucleic acid.

FIG. 13 depicts different binding constructs engineered to target miR-132, miR-155, miR-18a, and miR-17.

FIGS. 14A-D depicts a luciferase reporter assay using a dual reporter construct with six perfect or imperfect miRNA binding sites inserted into the 3′-UTR of the Renilla luciferase gene to determine the effect of engineered polynucleotides with different miRNA binding site types targeting miR-155 (FIG. 14A), miR-132 (FIG. 14B), miR-17 (FIG. 14C) and miR-18a (FIG. 14D). The figures depict imperf A/G/C/U as having deletion+at positions 9-11 and having an imperfect seed by inserting either A/G/C/U at position 6 from the 3′ end of the target site.

FIGS. 15A-D depict graphical representations of RNA minimum free energy (MFE) secondary structures of snowflake designs carrying different types of miRNA binding sites targeting miR-155 (FIG. 15A), miR-132 (FIG. 15B), miR-17 (FIG. 15C) and miR-18a (FIG. 15D).

FIG. 16 depicts DNA constructs wherein a DNA construct is delivered as a nucleic acid which is to be transcribed into an engineered nucleic acid. Engineered RNAs can be expressed by a CAG promoter, a CMV promoter, and a T7 promoter.

FIG. 17 depicts snowflake RNA (sfRNA) targeting miR-155 with different numbers of binding sites (e.g., x1 is equal to one binding site while x6 is equal to six binding sites). Additionally, a luciferase rescue reporter assay of equimolar amounts of sfRNA constructs with different binding sites and their ability to sponge miR-155 mimics from inhibiting luciferase expression is shown. Values represent mean±SD. Unless otherwise indicated, * denotes statistical significance relative to mimics (p<0.05:1-way ANOVA; n=3).

FIGS. 18A-D depicts different amounts of sfRNA with different numbers of binding sites. FIG. 18A shows a comparison of an equal number of binding sites (60) presented by sfRNA targeting miR-155 with different binding capacity (e.g., 60 moles of one binding site has the same total number of sites as 30 moles of two binding sites). FIGS. 18B-D shows a luciferase reporter assay of sfRNA with different binding capacity transfected at different molar amounts (equal mass) to achieve equal number of binding sites. The graphs show the constructs ability to sponge miR-155 mimics from inhibiting luciferase expression at different concentrations. Values represent mean±SD. * denotes statistical significance relative to mimics (p<0.05; 1-way ANOVA; n=3).

FIGS. 19A-B depicts 3 binding site sfRNA targeting miR-155 varying in linker size. FIG. 19A shows the predicted structure of the sfRNA. FIG. 19B shows a luciferase rescue reporter assay of 3 binding site sfRNA targeting miR-155 varying in linker size. The graph shows the constructs ability to sponge miR-155 mimics from inhibiting luciferase expression at different concentrations. Values represent mean±SD. Unless otherwise indicated, * denotes statistical significance relative to mimics (p<0.05:1-way ANOVA; n=3).

FIGS. 20A-B depicts 6 binding site sfRNA targeting miR-155 varying in linker size. FIG. 20A shows the predicted structure of the sfRNA. FIG. 20B shows a luciferase rescue reporter assay of 6 binding site sfRNA targeting miR-155 varying in linker size. The graph shows the constructs ability to sponge miR-155 mimics from inhibiting luciferase expression at different concentrations. Values represent mean±SD. Unless otherwise indicated. * denotes statistical significance relative to mimics (p<0.05:1-way ANOVA; n=3).

FIGS. 21A-B depicts 3 binding site sfRNA targeting miR-155 varying in homology arm types. FIG. 21A shows the predicted structure of the sfRNA. FIG. 21B shows a luciferase rescue reporter assay of 3 binding site sfRNA targeting miR-155 varying in homology arm types. Different constructs (12 and 15 nt homology arms) comprising no base deletion in the arms (ND), a single nt base deletion in one stem loop arm (BD), and a mismatch in the stem loop arm (MM) were analysed. The graph shows the constructs ability to sponge miR-155 mimics from inhibiting luciferase expression at different concentrations. Values represent mean±SD. * denotes statistical significance as indicated (p<0.05:1-way ANOVA; n=3).

FIGS. 22A-B depicts of 6 binding site sfRNA targeting miR-155 varying in homology arm type. FIG. 22A shows the predicted structure of the sfRNA. FIG. 22B shows a luciferase rescue reporter assay of 6 binding site sfRNA targeting miR-155 varying in homology arm type. Different constructs (12 and 15 nt homology arms) comprising no base deletion in the arms (ND), a single nt base deletion in one stem loop arm (BD), and a mismatch in the stem loop arm (MM) were analysed. The graph shows the constructs ability to sponge miR-155 mimics from inhibiting luciferase expression at different concentrations. Values represent mean±SD. * denotes statistical significance as indicated (p<0.05:1-way ANOVA; n=3).

FIGS. 23A-B depicts 3 binding site sfRNA targeting miR-155 varying in binding site type. FIG. 23A shows a luciferase rescue reporter assay of 3 binding site sfRNA targeting miR-155 varying in binding site type. Perf ctrl refers to a 100% complementarity against miR-155. Imperf ctrl refers to having one deletion and two mismatches at positions 9-11 from the 3′ end of the binding site. FIG. 23B shows a luciferase rescue reporter assay. Perf A/G/C/U refer to an imperfect seed by inserting either A/G/C/U nucleotide at position 6 from the 3′ end of the binding site. Imperf A/G/C/U refer to having one deletion and two mismatches at positions 9-11 and having an imperfect seed by inserting either A/G/C/U nucleotide at position 6 from the 3′ end of the binding site. Perf pos x refers to an imperfect seed with a mismatch at position x from the 3′end of the binding site. Imperf pos x refer to having one deletion and two mismatches at positions 9-11 and an imperfect seed by with a mismatch at position x from the 3′end of the binding site. The graph shows the constructs ability to sponge miR-155 mimics from inhibiting luciferase expression at different concentrations. FIG. 23A shows a comparison between Perf ctrl and Imperf ctrl binding sites. FIG. 23B shows a comparison across all binding sites. Values represent mean±SD. * denotes statistical significance relative to mimics (p<0.05:1-way ANOVA; n=3) for FIG. 23A. * denotes statistical significance comparing all perf sites to perf ctrl and imperf sites to imperf ctrl (p<0.05:1-way ANOVA; n=3) for FIG. 23B.

FIGS. 24A-B depicts 6 binding site sfRNA targeting miR-155 varying in binding site type. FIG. 24A Luciferase rescue reporter assay of 6 binding site sfRNA targeting miR-155 varying in binding site type. Perf ctrl refers to a 100% complementarity against miR-155. Imperf ctrl refers to having one deletion and two mismatches at positions 9-11 from the 3′ end of the binding site. FIG. 24B shows a luciferase rescue reporter assay. Perf A/G/C/U refer to an imperfect seed by inserting either A/G/C/U nucleotide at position 6 from the 3′ end of the binding site. Imperf A/G/C/U refer to having one deletion and two mismatches at positions 9-11 and having an imperfect seed by inserting either A/G/C/U nucleotide at position 6 from the 3′ end of the binding site. Perf pos x refers to an imperfect seed with a mismatch at position x from the 3′end of the binding site. Imperf pos x refer to having one deletion and two mismatches at positions 9-11 and an imperfect seed by with a mismatch at position x from the 3′end of the binding site. The graph shows the constructs ability to sponge miR-155 mimics from inhibiting luciferase expression at different concentrations. FIG. 24A shows a comparison between Perf ctrl and Imperf ctrl binding sites. FIG. 24B shows a comparison across all binding sites. Values represent mean±SD. * denotes statistical significance relative to mimics (p<0.05:1-way ANOVA; n=3) for FIG. 24A. * denotes statistical significance comparing all perf sites to perf ctrl and imperf sites to imperf ctrl (p<0.05; 1-way ANOVA; n=3) for FIG. 24B.

FIGS. 25A-B depicts 3 binding site sfRNA targeting miR-155 varying in auxiliary arm length. FIG. 25A shows the predicted structure of the sfRNA. FIG. 25B shows a luciferase rescue reporter assay of 3 binding site sfRNA targeting miR-155 varying in auxiliary arm length. Different constructs comprising of the following auxiliary arm lengths were analyzed: 4 nt pair (4/4), 6 nt pair (6/6), 8 nt pair (8/8), 12 nt pair (12/12) and 11 nt and 12 nt pair (11/12). The graph shows the constructs ability to sponge miR-155 mimics from inhibiting luciferase expression at different concentrations. Unless otherwise indicated, * denotes statistical significance relative to mimics (p<0.05:1-way ANOVA; n=3).

FIGS. 26A-B depicts 6 binding site sfRNA targeting miR-155 varying in auxiliary arm length. FIG. 26A shows the predicted structure of the sfRNA. FIG. 26B shows a luciferase rescue reporter assay of 6 binding site sfRNA targeting miR-155 varying in auxiliary arm length. Different constructs comprising of the following auxiliary length were analysed: 4 nt pair (4/4), 6 nt pair (6/6), 8 nt pair (8/8), 12 nt pair (12/12) and 11 nt and 12 nt pair (11/12). The graph shows the constructs ability to sponge miR-155 mimics from inhibiting luciferase expression at different concentrations. Unless otherwise indicated, * denotes statistical significance relative to mimics (p<0.05:1-way ANOVA; n=3).

FIG. 27 depicts (sfRNA) targeting miR-132 with different numbers of binding sites. FIG. 27 depicts the structure of snowflake RNA (sfRNA) targeting miR-132 with different numbers of binding sites (e.g., x1 is equal to one binding site while x6 is equal to six binding sites). Additionally, FIG. 27 shows a luciferase rescue reporter assay of equimolar amounts of sfRNA constructs with different binding sites and their ability to sponge miR-132 mimics from inhibiting luciferase expression is shown. Values represent mean±SD. * indicates statistical significance relative to mimics (p<0.05:1-way ANOVA; n=3).

FIG. 28A-C depicts a comparison of an equal number of binding sites (60) presented by sfRNA targeting miR-132 with different binding capacity (e.g., 60 moles of one binding site has the same total number of sites as 30 moles of two binding sites). Luciferase reporter assay of sfRNA with different binding capacity transfected at different molar amounts (equal mass) to achieve equal number of binding sites; FIG. 28A shows 50 ng. FIG. 28B shows 100 ng, and FIG. 28C shows 300 ng. The graphs show the constructs ability to sponge miR-132 mimics from inhibiting luciferase expression at different concentrations. Values represent mean±SD. Unless otherwise indicated, * denotes statistical significance relative to mimics (p<0.05:1-way ANOVA; n=3).

FIGS. 29A-B depicts 3 binding site sfRNA targeting miR-132 varying in linker size. FIG. 29A shows the predicted structure of the sfRNA. FIG. 29B shows a luciferase rescue reporter assay of 3 binding site sfRNA targeting miR-132 varying in linker size. The graph shows the constructs ability to sponge miR-132 mimics from inhibiting luciferase expression at different concentrations. Values represent mean±SD. Unless indicated. * denotes statistical significance relative to mimics (p<0.05:1-way ANOVA; n=3).

FIGS. 30A-B depicts 6 binding site sfRNA targeting miR-132 varying in linker size. FIG. 30A shows the predicted structure of the sfRNA. FIG. 30B shows a luciferase rescue reporter assay of 6 binding site sfRNA targeting miR-132 varying in linker size. The graph shows the constructs ability to sponge miR-132 mimics from inhibiting luciferase expression at different concentrations. Values represent mean±SD. Unless otherwise indicated, * denotes statistical significance relative to mimics (p<0.05:1-way ANOVA; n=3).

FIGS. 31A-B depicts 3 binding site sfRNA targeting miR-132 varying in homology arm types. FIG. 31A shows the predicted structure of the sfRNA. FIG. 31B shows a luciferase rescue reporter assay of 3 binding site sfRNA targeting miR-132 varying in homology arm types. Different constructs (12 and 15 nt homology arms) comprising no base deletion in the arms (ND), a single nt base deletion in one stem loop arm (BD), and a mismatch in the stem loop arm (MM) were analysed. The graph shows the constructs ability to sponge miR-132 mimics from inhibiting luciferase expression at different concentrations. * denotes statistical significance as indicated (p<0.05:1-way ANOVA; n=3).

FIGS. 32A-B depicts 6 binding site sfRNA targeting miR-132 varying in homology arm type. FIG. 32A shows the predicted structure of the sfRNA. FIG. 32B shows a luciferase rescue reporter assay of 6 binding site sfRNA targeting miR-132 varying in homology arm type. Different constructs (12 and 15 nt homology arms) comprising no base deletion in the arms (ND), a single nt base deletion in one stem loop arm (BD), and a mismatch in the stem loop arm (MM) were analysed. The graph shows the constructs ability to sponge miR-132 mimics from inhibiting luciferase expression at different concentrations. * denotes statistical significance as indicated (p<0.05:1-way ANOVA; n=3).

FIGS. 33A-B depicts 3 binding site sfRNA targeting miR-132 varying in binding site type. FIG. 33A shows a luciferase rescue reporter assay of 3 binding site sfRNA targeting miR-132 varying in binding site type. Perf ctrl refers to a 100% complementarity against miR-132. Imperf ctrl refers to having one deletion and two mismatches at positions 9-11 from the 3′ end of the binding site. FIG. 33B shows a luciferase rescue reporter assay. Perf A/G/C/U refer to an imperfect seed by inserting either A/G/C/U nucleotide at position 6 from the 3′ end of the binding site. Imperf A/G/C/U refer to having one deletion and two mismatches at positions 9-11 and having an imperfect seed by inserting either A/G/C/U nucleotide at position 6 from the 3′ end of the binding site. Perf pos x refers to an imperfect seed with a mismatch at position x from the 3′end of the binding site. Imperf pos x refer to having one deletion and two mismatches at positions 9-11 and an imperfect seed by with a mismatch at position x from the 3′end of the binding site. The graph shows the constructs ability to sponge miR-132 mimics from inhibiting luciferase expression at different concentrations. FIG. 33A shows a comparison between Perf ctrl and Imperf ctrl binding sites. FIG. 33B shows a comparison across all binding sites. Values represent mean±SD. * denotes statistical significance relative to mimics (p<0.05:1-way ANOVA; n=3) for FIG. 33A. * denotes statistical significance comparing all perf sites to perf ctrl and imperf sites to imperf ctrl (p<0.05:1-way ANOVA; n=3) for FIG. 33B.

FIGS. 34A-B depicts 6 binding site sfRNA targeting miR-132 varying in binding site type. FIG. 34A shows a luciferase rescue reporter assay of 6 binding site sfRNA targeting miR-132 varying in binding site type. Perf ctrl refers to a 100% complementarity against miR-132. Imperf ctrl refers to having one deletion and two mismatches at positions 9-11 from the 3′ end of the binding site. FIG. 34B shows a luciferase rescue reporter assay. Perf A/G/C/U refer to an imperfect seed by inserting either A/G/C/U nucleotide at position 6 from the 3′ end of the binding site. Imperf A/G/C/U refer to having one deletion and two mismatches at positions 9-11 and having an imperfect seed by inserting either A/G/C/U nucleotide at position 6 from the 3′ end of the binding site. Perf pos x refers to an imperfect seed with a mismatch at position x from the 3′end of the binding site. Imperf pos x refer to having one deletion and two mismatches at positions 9-11 and an imperfect seed by with a mismatch at position x from the 3′end of the binding site. The graph shows the constructs ability to sponge miR-132 mimics from inhibiting luciferase expression at different concentrations. FIG. 34A shows a comparison between Perf ctrl and Imperf ctrl binding sites. FIG. 34B shows a comparison across all binding sites. Values represent mean±SD. * denotes statistical significance relative to mimics (p<0.05:1-way ANOVA; n=3) for FIG. 34A. * denotes statistical significance comparing all perf sites to perf ctrl and imperf sites to imperf ctrl (p<0.05; 1-way ANOVA; n=3) for FIG. 34B.

FIGS. 35A-B depicts 3 binding site sfRNA targeting miR-132 varying in auxiliary arm length. FIG. 35A shows the predicted structure of the sfRNA. FIG. 35B shows a luciferase rescue reporter assay of 3 binding site sfRNA targeting miR-132 varying in auxiliary arm length. Different constructs comprising of the following auxiliary length were analysed: 4 nt pair (4/4), 6 nt pair (6/6), 8 nt pair (8/8), 12 nt pair (12/12) and 11 nt and 12 nt pair (11/12). The graph shows the constructs ability to sponge miR-132 mimics from inhibiting luciferase expression at different concentrations. Values represent mean±SD. Unless otherwise indicated, * denotes statistical significance relative to mimics (p<0.05:1-way ANOVA; n=3).

FIGS. 36A-B depicts 6 binding site sfRNA targeting miR-132 varying in auxiliary arm length. FIG. 36A shows the predicted structure of the sfRNA. FIG. 36B shows a luciferase rescue reporter assay of 6 binding site sfRNA targeting miR-132 varying in auxiliary arm length. Different constructs comprising of the following auxiliary length were analysed: 4 nt pair (4/4), 6 nt pair (6/6), 8 nt pair (8/8), 12 nt pair (12/12) and 11 nt and 12 nt pair (11/12). The graph shows the constructs ability to sponge miR-132 mimics from inhibiting luciferase expression at different concentrations. Values represent mean±SD. Unless otherwise indicated, * denotes statistical significance relative to mimics (p<0.05:1-way ANOVA; n=3).

FIG. 37 shows RT-PCR analysis of immune regulators, RIG-I, MDA5, OAS1, OASL, PKR, IFNb mRNA expression in HEK293T cells following transfection with modified 3 site sfRNAs targeting miR-155. Unmodified GTP refers to unmodified sfRNAs with a 5′ triphosphate end. sfRNAs with modified NTPS were synthesized with a 5′ triphosphate end. 100% 5mCTP refers to a 100% substitution of CTP with 5mCTP, 100% Ψ-UTP refers to a 100% substitution of UTP with Ψ-UTP, 100% 5mCTP+100% Ψ-UTP refers to a 100% substitution of both CTP and UTP with 5mCTP and Ψ-UTP, 100% substitution me1Ψ-UTP refers to a 100% substitution of UTP with me1Ψ-UTP, 10% and 100% m6ATP refers to a 10% or 100% substitution of ATP with m6ATP respectively. Values represent mean±SD.

FIG. 38 shows RT-PCR analysis of immune regulators, RIG-I, MDA5, OAS1, OASL, PKR, IFNb mRNA expression in HEK293T cells following transfection with modified 3 site sfRNAs targeting miR-155. Unmodified GTP refers to unmodified sfRNAs with a 5′ triphosphate end while unmodified GMP refers to unmodified sfRNAs with a 5′ monophosphate end. sfRNAs with modified NTPS were synthesized with a 5′ monophosphate end. Substitution with modified NTPs are described above. Values represent mean±SD.

FIG. 39 shows RT-PCR analysis of immune regulators, RIG-I, MDA5, OAS1, OASL, PKR, IFNb mRNA expression in HEK293T cells following transfection with modified 3 site sfRNAs targeting miR-132. Unmodified GTP refers to unmodified sfRNAs with a 5′ triphosphate end. sfRNAs with modified NTPS were synthesized with a 5′ triphosphate end. Substitution with modified NTPs are described above. Values represent mean±SD.

FIG. 40 shows RT-PCR analysis of immune regulators, RIG-I, MDA5, OAS1, OASL, PKR, IFNb mRNA expression in HEK293T cells following transfection with modified 3 site sfRNAs targeting miR-132. Unmodified GTP refers to unmodified sfRNAs with a 5′ triphosphate end while unmodified GMP refers to unmodified sfRNAs with a 5′ monophosphate end. sfRNAs with modified NTPS were synthesized with a 5′ monophosphate end. Substitution with modified NTPs are described above. Values represent mean±SD.

FIG. 41 depicts 3 and 6 binding site structured sfRNA and 3 and 6 binding site unstructured RNA configurations.

FIG. 42 depicts a graph of 3 and 6 binding site structured sfRNAs and 3 and 6 binding site unstructured RNA targeting miR-155. The Renilla/Firefly luciferase ratio is shown on the Y-axis for luciferase expression of a reporter. The X-axis depicts the different constructs: x3 uRNA is 3 binding site unstructured RNA; x3 sfRNA is 3 binding site structured sfRNA; x6 uRNA is 6 binding site unstructured RNA; x6 sfRNA is 6 binding site structured sfRNA. The graph shows the constructs ability to sponge miR-155 mimics from inhibiting luciferase expression.

FIG. 43 shows an in vitro binding assay to determine the specific interaction of a 3 binding site structured sfRNA with 12 nt stem loop arm designed to bind to miR-155. The far left of the gel depicts an RNA ladder and the next two columns are different amounts of miR-132 single-stranded oligo. The remaining columns from left to right are loaded with an unhybridised sfRNA or hybridised samples of the sfRNA with different amounts of either miR-132 or miR-155 single-stranded oligo. The last two columns are a negative control of GFP targeting sfRNA mixed with and without miR-155 single-stranded oligo. The gel shows the sfRNA, carrying a 12 nt homology arm size containing no deletion, is specific for the intended miRNA sequence, miR-155.

FIG. 44 shows an in vitro binding assay to determine the specific interaction of a 3 binding site structured sfRNA with 15 nt stem loop arm designed to bind to miR-155. The first four columns on the far left of the gel depicts different amounts of miR-132 and miR-155 single-stranded oligo. The remaining columns from left to right are loaded with an unhybridised sfRNA or hybridised samples of the sfRNA with different amounts of either miR-132 or miR-155 single-stranded oligo. The last column on the far right shows an RNA ladder. The gel shows the sfRNA, carrying a 15 nt homology arm size containing a single base deletion in one strand of an homology arm, is specific for the intended miRNA sequence, miR-155.

FIG. 45 shows an in vitro binding assay to determine the specific interaction of a 3 binding site structured sfRNA with 15 nt stem loop arm designed to bind to miR-132. The far left of the gel depicts an RNA ladder and the next four columns are different amounts of miR-132 and miR-155 single-stranded oligo. The remaining columns from left to right are loaded with an unhybridised sfRNA or hybridised samples of the sfRNA with different amounts of either miR-132 or miR-155 single-stranded oligo. The gel shows the sfRNA designed to sponge miR-132, carrying a 15 nt homology arm size containing a single base deletion in one strand of an homology arm, is specific for the intended miRNA sequence, miR-132.

FIG. 46 shows a luciferase rescue reporter assay of 3 binding site sfRNA targeting miR-155 varying in nucleotide modifications. Unmodified refers to unmodified sfRNAs. sfRNAs with modified NTPS were synthesized with a 5′ triphosphate end. 100% 5mCTP refers to a 100% substitution of CTP with 5mCTP, 100% Ψ-UTP refers to a 100% substitution of UTP with Ψ-UTP, 100% 5mCTP+100% Ψ-UTP refers to a 100% substitution of both CTP and UTP with 5mCTP and Ψ-UTP, 100% substitution me1Ψ-UTP refers to a 100% substitution of UTP with me1Ψ-UTP, 10% and 100% m6ATP refers to a 10% or 100% substitution of ATP with m6ATP respectively. All sfRNAs were synthesized with a 5′ triphosphate end. The graph shows the constructs ability to sponge miR-155 mimics from inhibiting luciferase expression. Unless otherwise indicated, * denotes statistical significance relative to mimics (p<0.05:1-way ANOVA; n=3).

FIG. 47 shows a luciferase rescue reporter assay of 3 binding site sfRNA targeting miR-155 varying in nucleotide modifications. Unmodified refers to unmodified sfRNAs. Substitution with modified NTPs are described above. All sfRNAs were synthesized with a 5′ monophosphate end. The graph shows the constructs ability to sponge miR-155 mimics from inhibiting luciferase expression. Unless otherwise indicated, * denotes statistical significance relative to mimics (p<0.05:1-way ANOVA; n=3).

FIG. 48 shows a luciferase rescue reporter assay of 3 binding site sfRNA targeting miR-132 varying in nucleotide modifications. Unmodified refers to unmodified sfRNAs. Substitution with modified NTPs are described above. All sfRNAs were synthesized with a 5′ triphosphate end. The graph shows the constructs ability to sponge miR-132 mimics from inhibiting luciferase expression. Unless otherwise indicated, * denotes statistical significance relative to mimics (p<0.05:1-way ANOVA; n=3).

FIG. 49 shows a luciferase rescue reporter assay of 3 binding site sfRNA targeting miR-132 varying in nucleotide modifications. Unmodified refers to unmodified sfRNAs. Substitution with modified NTPs are described above. All sfRNAs were synthesized with a 5′ monophosphate end. The graph shows the constructs ability to sponge miR-132 mimics from inhibiting luciferase expression. Unless otherwise indicated, * denotes statistical significance relative to mimics (p<0.05:1-way ANOVA; n=3).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

As used herein, the singular forms “a”, “an”, and “the” include plural references unless the context clearly dictates otherwise. Any reference to “or” herein may be intended to encompass “and/or” unless otherwise stated.

As used herein, the term “about” may mean the referenced numeric indication plus or minus: 5%, 10%, 15%, or 20% of that referenced numeric indication. In some instances, “about” may mean the referenced numeric indication plus or minus 10% of that referenced numeric indication. In some instances, “about” may mean the referenced numeric indication plus or minus 15% of that referenced numeric indication. In some instances, “about” may mean the referenced numeric indication plus or minus 20% of that referenced numeric indication.

The term “fragment,” as used herein, may be a portion of a sequence, a subset that may be shorter than a full-length sequence. A fragment may be a portion of a gene. A fragment may be a portion of a peptide or protein. A fragment may be a portion of an amino acid sequence. A fragment may be a portion of an oligonucleotide sequence. A fragment may be less than about: 20, 30, 40, 50 amino acids in length. A fragment may be about 10%, about 15%, about 20%, about 25%, about 30%, about 35%, about 40%, about 50%, about 60% or about 70% of the total length of an amino acid sequence or a nucleotide sequence. A fragment may be less than about: 20, 30, 40, 50 oligonucleotides in length.

As used herein, the term “miRNA” and “microRNA” are interchangeable. miRNA can refer to a small single-stranded non-coding RNA molecule that functions in RNA silencing and posttranscriptional regulation of gene expression. In some cases, “miRNA” may be further abbreviated as “miR”.

The phrases “nucleic acid” or “nucleic acid sequence,” as used herein, can refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. Nucleic acid molecules can be naturally occurring, recombinant, or synthetic. The term “nucleotide” may be further abbreviated as “nt”. The abbreviation “NA.” as used herein can refer to a nucleic acid. Any nucleotide sequence disclosed herein comprises the DNA sequence and the RNA sequence (wherein all T's in the DNA sequence are substituted for U's). In some cases, the DNA sequence disclosed herein encodes the RNA sequence, for example a DNA sequence herein can encode an engineered nucleic acid such as a sfRNA.

As used herein, “nucleobases”, also known as nitrogenous bases or often simply bases, may refer to nitrogen-containing biological compounds that form nucleosides, which, in turn, may be components of nucleotides, Nucleobases may form base pairs and may stack one upon another leading to long-chain helical structures such as ribonucleic acid (RNA) and deoxyribonucleic acid (DNA). Five nucleobases are typically found in nucleic acids. These may include adenine (A), cytosine (C), guanine (G), thymine (T), and uracil (U), Four of these nucleobases, namely adenine, cytosine, guanine, and thymine, are typically found in DNA. In RNA, the four bases typically found are adenine, cytosine, guanine, and uracil. Typically, in a Watson-Crick arrangement, adenine, which is a purine nucleobase, typically forms two hydrogen bonds with thymine, a pyrimidine nucleobase, in a double stranded DNA arrangement. Likewise, in a Watson-Crick arrangement, typically forms three hydrogen bonds with cytosine, another pyrimidine nucleobase in a double stranded DNA arrangement. In the case of RNA, uracil is typically substituted with uracil, another pyrimidine base such that adenine typically forms two hydrogen bonds with uracil. In certain instances, nucleobases can form non Watson-Crick pairing where one nucleobase that typically does not interact with and hydrogen bind with another nucleobase, nevertheless does so. Examples of mismatch pairing include guanine-guanine, guanine-adenine, guanine-thymine, guanine-uracil, adenine-guanine, adenine-cytosine.

As used herein, “nucleosides” can be glycosylamines that can be thought of as nucleotides without a phosphate group. A nucleoside may comprise a nucleobase (also termed a nitrogenous base) and a five-carbon sugar (ribose or 2′-deoxyribose) whereas a nucleotide may comprise a nucleobase, a five-carbon sugar, and one or more phosphate groups. In a nucleoside, the anomeric carbon may be linked through a glycosidic bond to the N9 of a purine or the N1 of a pyrimidine.

As used herein, the term “oligonucleotide”, can refer to a nucleic acid sequence of at least about 6 nucleotides to about 60 nucleotides, about 15 to about 30 nucleotides, or about 20 to 25 nucleotides, which can be used in PCR amplification or in a hybridization assay or microarray. As used herein, the term “oligonucleotide” can be substantially equivalent to the terms “amplimers,” “primers,” “oligomers,” and “probes,” as these terms are commonly defined in the art.

In some cases, a variant can refer to a nucleic acid sequence (e.g., polynucleotide) that differs from a reference nucleic acid sequence. In some cases, a variant can retain one or more properties of a nucleic acid sequence. For example, a variant may have about 70%, 75%, 80%, 85%, 90%, 95%, 96%, 97%, 98%, or 99% sequence identity compared to a reference polynucleotide sequence. A variant polynucleotide may differ from a reference polynucleotide by one or more modifications (e.g., substitutions, additions, and/or deletions). A variant polynucleotide and reference polynucleotide may differ in nucleotide sequence by one or more changes (e.g., mutations, additions, and/or deletions). A substituted or inserted amino acid residue may or may not be one encoded by the genetic code. “Variant” can include functional and structural variants. In some instances, a chemical modification may be added to a nucleic acid such as a phosphodiester modification, methylation of a hydroxyl group, the presence of an epigenetically marked base, a deoxyribose sugar, or other such modifications.

As used herein, “sponging” may refer to the at least partial sequestration of a target RNA by at least a portion of a loop of a stem loop. The portion of the loop which engages a target RNA may be a binding site. As used herein, the term “sponge” may refer to sponging as a noun.

As used herein, a “nucleic acid sponge” may refer to an engineered nucleic acid which may contain one or more complementary binding sites to a target RNA. In some instances, the complementary binding sites are comprised in a portion of a loop of a stem loop comprised in the nucleic acid sponge. In some instances, the nucleic acid sponge can comprise a plurality of stem loops where each stem loop of the plurality of stem loops is connected to at least one other stem loop of the plurality of stem loops by at least one linker. The portion(s) of the loop of the stem loop that do not contain a binding site can be an Auxiliary Arm(s). Stem loops of a nucleic acid sponge can be covalently linked through one or more linkers, which can be polynucleotide sequences that attach or join two stem loops. The linkers can join stem loops together, for example, proximal to or at the end of the stems of the stem loops opposite to the end of the stems attached to the loops of the stem loops. In certain instances, a nucleic acid sponge's binding sites, which can be the same or different, can be independently specific to or selective for an miRNA seed sequence, thereby allowing for reducing or blocking the activity of, at least partial sequestration of, or both, of one or more target RNAs. In some cases, an engineered nucleic acid can have one, two, three, or more binding sites within a loop of a stem loop of an engineered RNA. In some cases, the binding sites within a loop of a stem loop can be the same or they can be different.

As used herein, a “snowflake” may refer an engineered nucleic acid or a nucleic acid sponge, that, when depicted and viewed two dimensionally takes has a snowflake-like appearance. The snowflake can, for example, appear to have a central circular hub comprising linkers and portions of stems of stem loops. As used herein, a “snowflake nucleic acid” is another term for snowflake. In some cases, a snowflake nucleic acid can be an RNA. In some cases, a snowflake RNA can be called a sfRNA.

As used herein, an “engineered nucleic acid-target RNA complex” can refer to a complex formed when a target RNA is bound, for example non-covalently bound, for example, through hydrogen bonding. Watson-Crick base pairing, Wobble Base Pairing, Van Der Waals interactions, or any combination thereof, to an engineered nucleic acid or nucleic acid sponge.

As used herein a target nucleic acid can be any nucleic acid. In some cases, a target nucleic acid can be a DNA or it can be an RNA. As used herein, a target RNA can be an RNA to which a nucleic acid sponge or engineered nucleic acid is engineered to bind. The binding of the nucleic acid sponge or engineered nucleic acid to the target RNA can be non-covalent. for example, through hydrogen bonding. Watson-Crick base pairing, Wobble Base Pairing, Van Der Waals interactions, or any combination thereof. A portion of the nucleic acid sponge or engineered nucleic acid, for example binding site of a loop of a stem loop of the nucleic acid sponge or engineered nucleic acid, can have at least partial complementarity to at least a portion of the target RNA. In some instances, the portion of the target RNA can be a seed sequence or seed region. Target RNAs can include a messenger RNA (mRNA), a ribosomal RNA (rRNA), a transfer RNA (IRNA), a ribozyme a transfer messenger RNA (tmRNA), a double stranded RNA (dsRNA), a small nuclear RNA (ssRNA), a small nucleolar RNA (snoRNA), a Piwi-interacting RNAs (piRNA), a microRNA (miRNA), a small interfering RNA (siRNA), a long non-coding RNA (lncRNA), or any combination thereof.

As used herein, a “seed sequence may refer to a conserved heptametrical sequence which may be situated at positions 2-7 from a 5′ miRNA end. As used herein a “seed region” can be another term for seed sequence.

As used herein, a “stem loop” can refer to an intramolecular base paring that may occur in a single stranded nucleic acid such as RNA. A stem loop can occur when two regions of the same strand, typically at least partially complementary in nucleotide sequences, when read in opposite directions, base pair to form a double helix that ends in a loop as a result of a substantially non-complementary sequence of nucleic acids between the two regions of the same strand which when read in opposite directions, base pair to form a double helix. The sequence of nucleic acids forming the loop does not typically form intramolecular base paring with itself. In some cases, at least a portion of the sequence of nucleic acids forming the loop can have intramolecular base paring with itself.

As used herein, a “stem” can refer to a portion of a stem loop that shares at least partial complementarity in nucleotide sequence when read in opposite directions, minus the substantially non-complementary nucleic acid sequence (e.g., the loop) between these two at least partially complementary nucleic acid sequences when read in opposite directions. In some cases, a stem can be referred to as an homology arm.

As used herein, a “loop” can refer to the substantially non-complementary sequence of a nucleic acid sequence between the two at least partially complementary sequences when read in opposite directions and which comprise the stem of the stem loop.

As used herein, a “binding sequence” can refer to a nucleic acid sequence within a loop designed to have at least partial complementarity to at least a portion of a target RNA or target nucleic acid.

As used herein, a “miRNA binding site” can refer to a location of the binding sequence within a loop of a stem loop adapted to have at least partial complementarity with a target RNA when the target RNA is miRNA.

As used herein an “auxiliary arm” can refer to a nucleic acid sequence within a loop that does not comprise a binding sequence. In some instances, two auxiliary arms flank a binding sequence within the loop.

As used herein, a “a nested stem loop” can refer to a sequence with at least two regions within a loop between the stem and the binding sequence of the loop with at least partial complementarity when read in opposite directions such that intramolecular base paring may occur. A nested stem loop can have substantially non-complementary sequences between its two regions of at least partial complementarity when read in opposite directions.

As used herein a “linker” can refer to a nucleic acid sequence, which can be single stranded, and that can covalently link two stem loops together. Linkers can join stem loops together, for example, proximal to or at the end of the stems of the stem loops opposite to the end of the stems attached to the loops of the stem loops. In some instances, a linker can join a 5′ end of one stem of a stem loop to a 3′ end of another stem of anther stem loop.

As used herein, a “hairpin” or “hairpin loop” can be a stem loop intramolecular base pairing that may occur in a single stranded nucleic acid such as RNA. Typically, a stem loop occurs when two regions of the same strand, typically at least partially complementary in nucleotide sequence when read in opposite directions, base pair to form a double helix that ends in an unpaired loop of the stem loop. The resulting structure may bind to or otherwise associate with for example, other nucleic acids.

As used herein, a “protrusion” may refer to a stem of a stem loop protruding from another stem of a stem loop.

As used herein, a “mismatch” can refer to a single nucleotide in a nucleic acid sponge that is unpaired to an opposing single nucleotide in a target RNA within an engineered acid-target RNA complex. A mismatch can comprise any two single nucleotides that do not base pair withing the engineered nucleic acid-target RNA complex. Examples of Mismatch pairing include guanine-guanine, guanine-adenine, guanine-thymine, guanine-uracil, adenine-guanine, adenine-cytosine.

As used herein, a “bulge can refer to a partially complementary binding between a binding sequence and a target RNA which may comprise at least one deletion and two mismatches at between the binding sequence and an at least partially complementary sequence of a target RNA.

As used herein “complementarity” can refer to non-covalent bonding of nucleobases through hydrogen bonding, Watson-Crick base pairing, Wobble Base Pairing, Van Der Waals interactions, or any combination thereof.

As used herein, “non-circularized” within the context of a nucleic acid sponge or engineered nucleic acid can refer to a terminal nucleotide or nucleoside at the 3′ end of the nucleic acid sponge or engineered nucleic acid is not covalently connected, either directly or via a linking group such as a phosphodiester group, to terminal nucleotide or nucleoside at the 5′ end of the nucleic acid sponge or engineered nucleic acid. In some instances, this can mean that one or two terminal nucleotides of the nucleic acid sponge or engineered nucleic acid individually contain a 3′ or a 5′ hydroxyl group, or both, that in an aqueous solution is exposed to the aqueous solution.

As used herein, a “wobble base pair” can refer to two nucleotide bases that weakly base pair when compared to the base pairing that occurs through Watson-Crick base pairing.

As used herein, a chemical modification” can refer to a modification of a nucleobase, a nucleoside, a nucleotide, a nucleic acid sequence, or a combination thereof through covalent modification such as a phosphodiester modification of a nucleic acid sequence, a methylation of a hydroxyl group of a nucleoside or nucleotide.

In some aspects, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology, similarity, or percent identity between two sequences. Any disclosed sequence herein also comprises sequences with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or sequence homology to the disclosed sequence. Any DNA sequence provided herein also includes the RNA sequence wherein all T's are substituted for U's. Any RNA sequence provided herein also includes the DNA sequence wherein all U's are substituted for T's. The two sequences can be genes, nucleotides, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=15, or can be varied (e.g., W=5 or W=20). Other examples include the algorithm of Myers and Miller, CABIOS (1989), ADVANCE, ADAM, BLAT, and FASTA. “Length homology” can in some instances be calculated by dividing the number of nucleotides in a first polynucleotide by the number of nucleotides in a second polynucleotide and multiplying the result by 100%—for example about: 80%, 81%, 82%, 83%, 84%, 85%, 86%, 87%, 88%, 89%, 90%, 91%, 92%, 93%, 94%, 95%, 96%, 97%, 98%, 99%, 100% homology to the disclosed sequence.

The phrase “pharmaceutically acceptable” may be employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” as used herein may refer to a pharmaceutically acceptable material, composition or vehicle, such as a liquid or solid filler, diluent, excipient, carrier, solvent or encapsulating material.

The terms “treat,” “treating” or “treatment,” as used herein, may include at least partially: alleviating, abating or ameliorating a disease or condition symptom; preventing an additional symptom: ameliorating or preventing the underlying causes of symptom: inhibiting the disease or condition, e.g., at least partially arresting the development of the disease or condition: relieving the disease or condition: causing regression of the disease or condition (e.g. causing a cancer to regress); relieving a condition caused by the disease or condition: or stopping a symptom of the disease or condition either prophylactically, therapeutically or both. Treatment may include stopping the growth of a cancer, reducing the size of a tumor, stopping an infection, reducing an infection, decreasing inflammation, preventing cancer, preventing an infection, or prolonging the life span of a subject when compared to an otherwise substantially identical subject who may not be treated.

As used herein, a “pharmaceutical agent” may refer to an agent or a therapy that may be used to prevent, diagnose, treat, or cure a disease, or combinations thereof. In some cases, a pharmaceutical agent can comprise engineered polynucleotide, a DNA encoding the engineered polynucleotide, or a vector containing or encoding the engineered polynucleotide. or, in some aspects, a method described herein may comprise administering a therapeutically effective amount of these to a subject, who can be a human or animal subject, who can be a mammal.

Included in the present disclosure may be salts, including pharmaceutically acceptable salts, of the compositions described herein. The compounds or compositions of the present disclosure that may possess a sufficiently acidic, a sufficiently basic, or both functional groups, may react with any of a number of in-organic bases, inorganic acids, or organic acids, to form a salt. Alternatively, compositions containing compounds that are inherently charged, such as those with quaternary nitrogen, may form a salt with an appropriate counterion, e.g., a halide such as bromide, chloride, or fluoride, particularly bromide.

As used herein, “agent” or “biologically active agent” may refer to a biological, pharmaceutical, or chemical compound or a salt of any of these. Non-limiting examples may include a simple or complex organic or inorganic molecule, a peptide, a protein, a nucleotide such as an engineered single stranded RNA, an engineered single stranded DNA, an alternative nucleic acid, a protein, a carbohydrate, a toxin, or a chemotherapeutic compound. Various compounds may be synthesized, for example, small molecules and oligomers (e.g., oligopeptides and oligonucleotides), or synthetic organic compounds based on various core structures. In addition, various natural sources may provide compounds for screening, such as plant or animal extracts, and the like.

Activity of a protein, as used herein, may refer to a transcript level of mRNA transcribed from a gene that codes for the protein, expression level of the protein, nature of an expressed protein, such as folding, ability of the protein to interact with other proteins in a tumor microenvironment, and ability of the protein to interact with downstream or upstream signaling molecules in a signaling cascade of which the protein is a member.

In some aspects, disclosed herein, compounds may be in a form of pharmaceutically acceptable salts. As well, active metabolites of these compounds having the same type of activity may be included in the scope of the present disclosure. In addition, the compounds described herein may exist in unsolvated as well as solvated forms with pharmaceutically acceptable solvents such as water, ethanol, and the like. The solvated forms of the compounds presented herein may also considered to be disclosed herein.

### Overview

Disclosed herein are engineered nucleic acids, polynucleotides encoding engineered nucleic acids, vectors encoding or containing engineered nucleic acids, methods of making engineered nucleic acids, and treating diseases with engineered nucleic acids. Engineered polynucleotides can function as sponges by, for example, binding and at least in part repressing the activity of microRNAs (miRNAs) and of other nucleic acids. In some cases, an engineered nucleic acid can comprise a snowflake-like geometric shape when drawn in two-dimensions. In some embodiments, an engineered nucleic acid can adopt or is configured to adopt a snowflake-like geometric shape. An engineered nucleic acid may act as a sponge and may be custom designed to target miRNA targets or other targets of interest. In some cases, an engineered nucleic acid can target a target RNA. The engineered nucleic acids can be used to treat or prevent diseases such as diseases associated with miRNAs. For example, engineered nucleic acids can target miR-17, miR-132, miR-18 and miR-155, which can be associated with a disease.

### Engineered Nucleic Acid Design

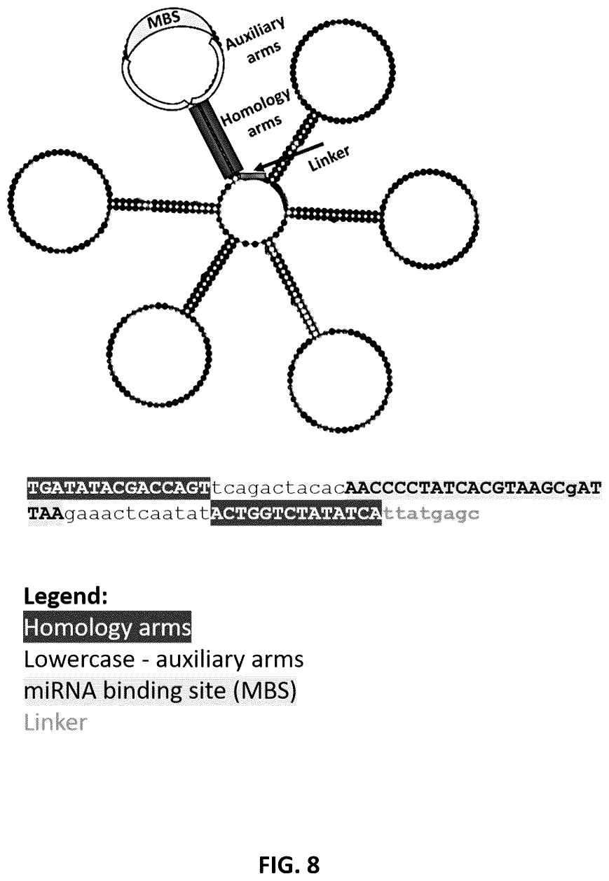

Certain aspects of the disclosure herein include engineered nucleic acids in the form of a snowflake-like configuration as shown in FIGS. 8, 11, 12. In general, an engineered nucleic acid, when viewed two dimensionally, can comprise a plurality of stem loops with some or all of the loops of the plurality of stem loops comprising a binding site, and with each stem loop of the plurality of stem loops connected to at least one other stem loop via a linker. Linkers can link stem loops proximal to an end of a stem of each stem loop linked by a linker that is opposite to a portion of a stem which contains a loop. In certain instances, the binding site binds to an RNA sequence such as a mRNA or a miRNA. In other instances, the binding site binds to a DNA sequence.

In general, engineered nucleic acids can function as sponges by binding and repressing the activity or accessibility of target RNA. Parameters such as number of engineered nucleic acid binding sites, spacer sizes, number of stem loops, number of nucleotides in loops of stem loops, and number of paired nucleotides in stems of stem loops can be modified to achieve sequestration of a designated target RNA. An engineered nucleic acid binding site can be configured to render the binding sites most accessible for binding target sequences. One or more stem-loops structures of the plurality of stem loops may comprise a single single-stranded nucleic acid target site and auxiliary single-stranded 5′ and 3′ extensions (auxiliary arms). In certain aspects, wherein the binding site is adapted to bind to a nucleic acid, the auxiliary arms can enable the formation of an A-form nucleic helix upon nucleic binding. In certain specific aspects, the nucleic acid comprising the auxiliary arms is RNA. Still further, in certain aspects, the binding site can be directed to bind or hybridize to a target miRNA. In other aspects, the binding site can be directed to bind or hybridize to a: messenger RNA (mRNA), ribosomal RNA (IRNA), transfer RNA (tRNA), ribozymes (RNA enzymes), transfer messenger RNA (tmRNA), double stranded RNA (dsRNA), small nuclear RNA (ssRNA), small nucleolar RNA (snoRNA), Piwi-interacting RNAs (piRNA), and long non-coding RNA (lncRNA).

In certain aspects regarding the engineered nucleic acid, the engineered nucleic acid can comprise one or more negatively charged stem-loops. In some instances, one or more loops of a plurality of stem loops can have a net negative charge (excluding the presence of counter cations). In some cases, the negatively charged stem-loops can comprise the auxiliary arms and the binding site. In certain instances, the stem loops can repel each other exposing the miRNA binding sites on the surface of the structure where they can be less affected by volume exclusion effects. A second advantage can be that the less structured miRNA binding sites can be approached more efficiently by miRNAs or RISCs as little, or no energy can be needed to open up any internal structure.

In some cases, the loops of a stem-loop of the engineered nucleic acid herein can be configured to bind to the same target nucleic acids. In some cases, the loops of a stem-loop of the engineered nucleic acid herein can be configured to bind to different target nucleic acids. In some cases, the binding site of a first loop of a stem loop in the engineered nucleic acid is configured to bind or hybridise to a second binding site of a second different stem loop in the engineered nucleic acid. In some cases, the binding site of a first loop of a stem loop in the engineered nucleic acid is configured to bind or hybridise to a second binding site of the same stem loop in the engineered nucleic acid.

In certain instances, the engineered nucleic acid herein can comprise a linker sequence, for example, as shown in FIGS. 8 and 11. Linker sequences can be single stranded RNA, DNA, or RNA and DNA nucleotide sequences spaced between stems of stem loops. They may contain or not contain one or more chemical modifications. In some instances, a linker sequence serves to connect a stem loop of the engineered nucleic acid to at least one other stem loop. The linkers can be 1, 2, 3, 4, 5, 6, 7, 8, 9, 10, 11, 12, 13, 14, 15, 16, 17, 18, 19, 20, 21, 22, 23, 24, 25, or more nucleotides in length. In some instances, linkers can be about: 2 to 10 nucleotides in length, 3 to 8 nucleotides in length, 4 to 15 nucleotides in length, or 6 to 25 nucleotides in length. In some cases, an engineered nucleic acid may have linkers of the same length. In some cases, an engineered nucleic acid may have linkers of different lengths. In certain instances, the linker sequences can be 2, 4, 6, or 8 nucleotides in length.

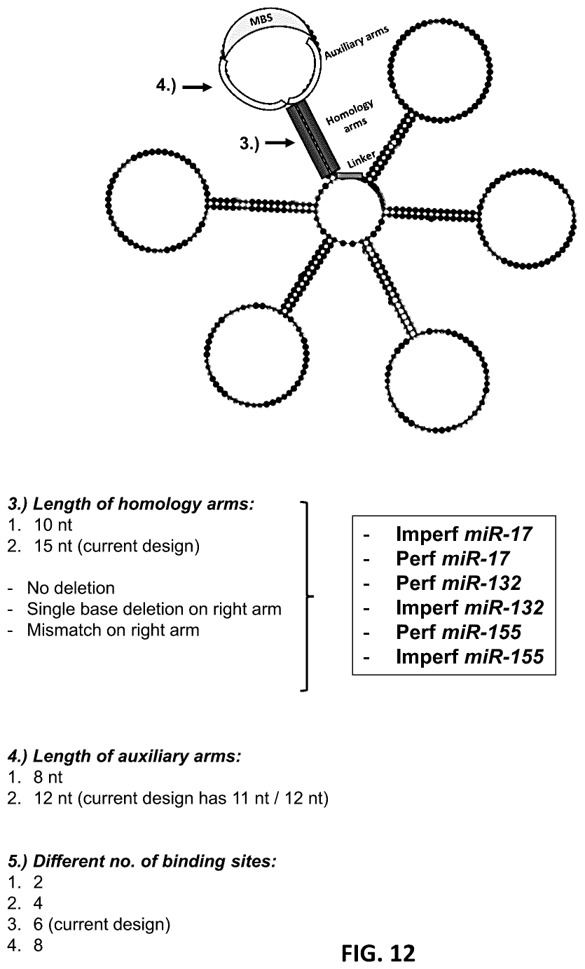

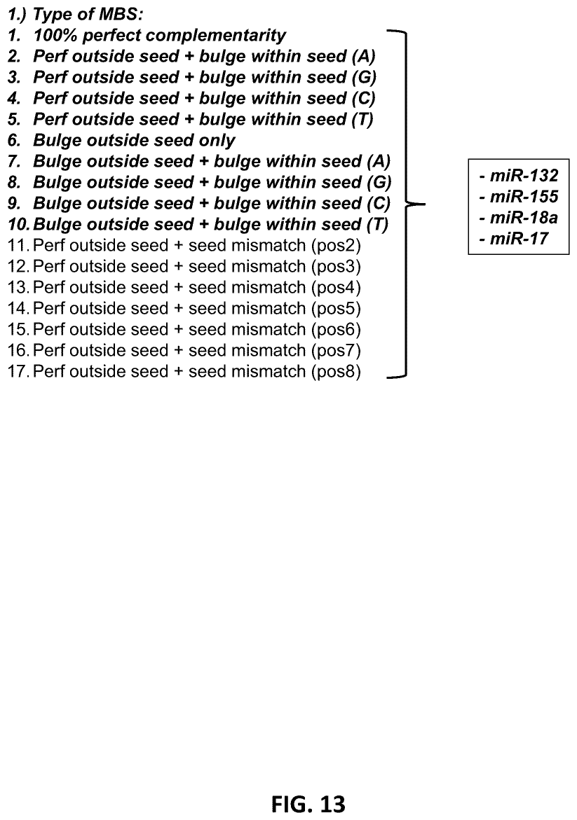

Additionally, engineered nucleic acids may comprise stems of stem loops which radiate outwardly as seen in FIGS. 8 and 12. An engineered nucleic acid sequence can comprise sets of complementary sequences such that when folded into a two-dimensional form, the resultant engineered nucleic acid comprises stems of stem loops with complementary base pairing between the sequences as shown. In certain cases, the base pairing is about: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary between the nucleic acid sequences that form a stem. The stems of stem loops can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In some instances, linkers, which link the 5′ end of one stem of a stem loop to the 3′ end of another stem loop, can be about: 2 to 100 nucleotides in length, 5 to 25 nucleotides in length, 3 to 12 nucleotides in length, 10 to 30 nucleotides in length, 2 to 8 nucleotides in length, 8 to 40 nucleotides in length, 15 to 50 nucleotides in length, or 20 to 70 nucleotides in length. In some cases, an engineered nucleic acid may have stems of stem loops of the same length. In some cases, an engineered nucleic acid may have stems of stem loops which are of different lengths. In certain aspects, the base pairing, which can be Watson-Crick base pairing, is 100% complementary. In some instances, one or more paired base pairs in one or more stems of the stem loops can independently pair via wobble base pairing. In some cases, a stem of a stem loop can be called a homology arm. In some cases, a stem of a stem loop can have 1 nucleotide, 2 nucleotide, 3 nucleotide, or more deletion in a stem loop arm. In some cases, a stem loop arm does not have a deletion. In some cases, a stem loop arm are can have 1, 2, 3, or more mismatches.

In some instances, a loop of a stem loop can be independently interrupted by a second stem which protrudes or projects out of the interrupted loop. The second stem can be different or the same in the number and composition of nucleotides as the stem. The end of the second stem that is distal from the interrupted loop can contain a further structural feature, for example, a second loop, or a bulge. If a second loop is present, the second loop can be different or the same in the number and composition of nucleotides to interrupted loop. If a bulge is present, the bulge can have the same number or a different number of nucleotides on each side of the bulge. Still further, in certain aspects, if a bulge is present, the end of the bulge that is distal to the second stem can contain a third stem, which can be identical or different in number and composition of nucleotides, to either or both of the stem and the second stem. The third stem can terminate, for example, in a third loop, which can be different or the same in number and composition of nucleotides as the interrupted loop. The number of paired nucleotides in the stem, the second stem, or the third stem can independently range from 1 to 50 paired nucleotides. The bulge, when present, can independently on each side of the bulge contain from about 1 to about 20 nucleotides. If the bulge contains only one nucleotide on each side of the bulge, then this is a mismatch. The second loop can independently contain from about 1 to about 50 nucleotides.

Engineered nucleic acids, when viewed from a two-dimensional perspective may comprise a geometric confirmation, that substantially displays C2, C3, C4, C5, C6, C7, C8, C9, C10, C11, C12, C13, C14, C15, C16, C17, C18, C19, C20 or more symmetries. In this regard, a stem loop may be substantially equidistant from each other stem loop. Still further, a stem loop may be substantially the same angle from each other when viewed in a substantially circularized form, such as, despite not having a 5′ end connected to a 3′ end, appears to have a central circular hub. For example, a C3 symmetry may have stem loops that are substantially 120 degrees from each other and a C4 symmetry may have stem loops that are substantially 90 degrees from each other.

Engineered polynucleotides herein can be not circularized. In some instances, this can mean that a terminal nucleotide or nucleoside at the 3′ end of the engineered polynucleotide is not covalently connected, either directly or via a linking group such as a phosphodiester group, to terminal nucleotide or nucleoside at the 5′ end of the engineered polynucleotide. In some instances, this can mean that one or two terminal nucleotides of the engineered polynucleotide individually contain a 3′ or a 5′ hydroxyl group that in an aqueous solution is exposed to the aqueous solution.

Engineered polynucleotides may comprise different phosphate ends selected for the purpose of triggering an innate immune response or inhibiting an innate immune response to an engineered nucleic acid which has been introduced into a cell for the purposes of sequestering target RNAs. In some instances, an engineered nucleic acid may carry a 5′ triphosphate group to trigger an innate immune response. In other instances, an engineered nucleic acid may carry a 5′ monophosphate group to prevent an innate immune response. In some cases, an engineered nucleic acid, such as a sfRNA can comprise a 5′ triphosphate end. In some cases, an engineered nucleic acid can comprise a 5′ Guanosine-5′-triphosphate (GTP) end. In some cases, an engineered nucleic acid can comprise a 5′ Guanosine monophosphate (GMP) end.

In some cases, synthesizing an engineered nucleic acid, such as a sfRNA can comprise synthesizing a sfRNA with modified nucleoside triphosphates (NTP) to reduce or alter an immune response. In some cases, synthesizing a modified NTP can comprise using 5-Methylcytidine-5′-Triphosphate (5mCTP), Pseudouridine-5′-triphosphate (Ψ-UTP) N1-Methylpseudo-UTP (me1Ψ-UTP), N6-Methyladenosine-5′-Triphosphate (m6ATP). In some cases, engineered nucleic acid can comprise a modified nucleotide or nucleoside. In some cases, a modified nucleotide or nucleoside can comprise pseudouridine, 5-methylcytidine, n1-methylpseudouridine, N6-methyladenosine, or any combination thereof. In some cases, an engineered nucleic acid can comprise about: 5% to about 100%, 10% to about 100%, 20% to about 50%, 30% to about 70%, 50% to about 80%, or 60% to about 100% substitution of uridine with pseudouridine in an engineered nucleic acid. In some cases, an engineered nucleic acid can comprise about: 5% to about 100%, 10% to about 100%, 20% to about 50%, 30% to about 70%, 50% to about 80%, or 60% to about 100% substitution of cytidine with 5-methylcytidine in an engineered nucleic acid. In some cases, an engineered nucleic acid can comprise about: 5% to about 100%, 10% to about 100%, 20% to about 50%, 30% to about 70%, 50% to about 80%, or 60% to about 100% substitution of uridine with n1-methylpseudouridine in an engineered nucleic acid. In some cases, an engineered nucleic acid can comprise about: 5% to about 100%, 10% to about 100%, 20% to about 50%, 30% to about 70%, 50% to about 80%, or 60% to about 100% substitution of adenosine with N6-methyladenosine in an engineered nucleic acid. In some cases, an engineered nucleic acid can comprise more than one modified nucleotides or nucleosides for example, a nucleic acid can comprise 5-methylcytidine and pseudouridine. In some cases, an engineered nucleic acid can comprise N-6-methyladenosine (m6A), pseudouridine (Ψ), N-1-methylpseudouridine (ml), 2 fluoro-deoxyuridine (2FdU), 5-methylcytidine (5mC), 5-hydroxymethylcytidine (5hmC), 5-methoxycytidine (5moC), 2 fluoro-deoxycytidine (2FdC), 2-thiouridine (s2u), 5-methoxyuridine (5moU), 5-methyluridine (5meU), N1-ethylpseudouridine (N1-et-Ψ), 5-carboxymethyluridine (cm5U), or any combination thereof. In some cases, a modified nucleotide or nucleoside can comprise N-6-methyladenosine (m6A), pseudouridine (Ψ). N-1-methylpseudouridine (mΨ), 2 fluoro-deoxyuridine (2FdU), 5-methylcytidine (5mC), 5-hydroxymethylcytidine (5hmC), 5-methoxycytidine (5moC), 2 fluoro-deoxycytidine (2FdC), 2-thiouridine (s2u), 5-methoxyuridine (5moU), 5-methyluridine (5meU), N1-ethylpseudouridine (N1-et-Ψ), 5-carboxymethyluridine (cm5U), or any combination thereof

Engineered nucleic acids disclosed herein can comprise auxiliary arms which extend outwardly from the stems of stem loops, for example, as seen in FIGS. 8 and 12. In some instances, auxiliary arm sequences are non-complementary and do not form secondary structures within the auxiliary arms themselves. In certain instances, each auxiliary arm can have an identical number of nucleotides. In other instances, there each auxiliary arm can have a difference in the number of nucleotides. In some cases, the auxiliary arms can have arm lengths of about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides in length. In some instances, auxiliary arms can be about: 2 to about 50 nucleotides in length, 5 to 20 nucleotides in length, 4 to 18 nucleotides in length, 10 to about 15 nucleotides in length, 8 to 16 nucleotides in length, 10 to 40 nucleotides in length, or 20 to 50 nucleotides in length. In some cases, auxiliary arms can have between 5 and 20 nucleotides when the binding site of the hairpin loop targets a nucleic acid. In certain instances, the auxiliary arms can be between 10 and 15 nucleotides. In some cases, an auxiliary arm can have one or more deletions or one or more mismatches.

In some embodiments, the engineered polynucleotide can have binding sites that sequester DNA sequences, or RNA sequences, or both. RNA sequences can include messenger RNA (mRNA), ribosomal RNA (rRNA), transfer RNA (tRNA), ribozymes (RNA enzymes) transfer messenger RNA (tmRNA), double stranded RNA (dsRNA), small nuclear RNA (ssRNA), small nucleolar RNA (snoRNA), Piwi-interacting RNAs (piRNA), and ling non-coding RNA (lncRNA). In certain instances, the target can be a miRNA as described or listed above. In certain instances, the binding site can have nucleotide lengths of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. In some instances, the binding site can be about: 10 to 100 nucleotides in length, 20 to 75 nucleotides in length, 10 to 80 nucleotides in length, 25 to 50 nucleotides in length, 30 to 65 nucleotides in length, 25 to 50 nucleotides in length, or 30 to 80 nucleotides in length. In some cases, a binding site can independently be between 10 and 70 nucleotides in length. In certain instances, a binding site can independently be between 40 and 60 nucleotides in length for binding to nucleic acids such as miRNAs.

In some embodiments, the binding site can have perfect (perf) complementarity or imperfect (imperf) complementary to a target nucleic acid. For example, a binding site may have a mismatch to a nucleotide in the target nucleic acid therefore it would have imperfect complementarity. In certain cases, the base pairing between and binding site and a target nucleic acid can be about: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary. In some cases, a target nucleic acid and a binding site can have complementarity in a region of about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some cases, a target nucleic acid and a binding site can have complementarity in a region of about: 5, 6, 7 or 8 nucleotides. In some cases, the target nucleic acid and the binding site can have multiple regions of complementarity, for example there can be 1, 2, 3, 4, or more than 5 regions of complementarity shared between the target nucleic acid and the binding site. In some cases, a target nucleic acid and a binding site can have a mismatch at one or more sites. In some cases, a target nucleic acid and a binding site can have 1, 2, 3, 4, or more than 5 mismatches. In some cases, a target nucleic acid and a binding site can have a deletion at one, two, three or more sites. For example, a binding site can have a deletion in its sequence as compared to the sequence in the target nucleic acid. In another example, a target nucleic acid can have a deletion in its sequence as compared to the binding site. In some instances, one, two, three, or more paired base pairs between the target nucleic acid and the binding site can independently pair via wobble base pairing.

In some embodiments, an engineered nucleic acid can comprise a mismatch to a target nucleic acid and a base of a mismatch can comprise a C, an A, a G, a T, or a U. In some cases, an target nucleic acid can comprise a mismatch to an engineered nucleic acid and a base of a mismatch can comprise a C, an A, a G, a T, or a U. In some cases, a mismatch can be an A/C mismatch, a A/G mismatch, a T/G mismatch, a T/C mismatch, a U/C mismatch, or a U/C mismatch. In some cases, an engineered nucleic acid can comprise one, two, three or more mismatches. In some cases, the mismatch can be an A/C mismatch where the A can be in the binding site of the loop of the stem loop of the engineered nucleic acid and the C can be in the target nucleic acid or the A can be in the target nucleic acid and the C can be in the binding site of the loop of the stem loop of the engineered nucleic acid. In some cases, the mismatch can be A/G mismatch where the A can be in the binding site of the loop of the stem loop of the engineered nucleic acid and the G can be in the target nucleic acid or the A can be in the target nucleic acid and the G can be in the binding site of the loop of the stem loop of the engineered nucleic acid. In some cases, the mismatch can be a T/G mismatch where the T can be in the binding site of the loop of the stem loop of the engineered nucleic acid and the G can be in the target nucleic acid or the T can be in the target nucleic acid and the G can be in the binding site of the loop of the stem loop of the engineered nucleic acid. In some cases, the mismatch can be a T/C mismatch where the T can be in the binding site of the loop of the stem loop of the engineered nucleic acid and the C can be in the target nucleic acid or the T can be in the target nucleic acid and the C can be in the binding site of the loop of the stem loop of the engineered nucleic acid. In some cases, the mismatch can be a U/G mismatch where the U can be in the binding site of the loop of the stem loop of the engineered nucleic acid and the G can be in the target nucleic acid or the U can be in the target nucleic acid and the G can be in the binding site of the loop of the stem loop of the engineered nucleic acid. In some cases, the mismatch can be a U/C mismatch where the U can be in the binding site of the loop of the stem loop of the engineered nucleic acid and the C can be in the target nucleic acid or the U can be in the target nucleic acid and the C can be in the binding site of the loop of the stem loop of the engineered nucleic acid. In some cases a U/G mismatch can be a wobble base pair.

In some embodiments, an engineered nucleic acid herein can comprise a length of about: 20 to about 1000 nucleotides, 20 to about 900 nucleotides, 20 to about 800 nucleotides, 20 to about 700 nucleotides, 20 to about 600 nucleotides, 20 to about 500 nucleotides, 50 to about 550 nucleotides, 50 to about 500 nucleotides, 50 to about 400 nucleotides, or about 100 to about 700 nucleotides. In some embodiments, an engineered nucleic acid herein can comprise a length of greater than, equal to, or less than about: 25, 30, 35, 40, 45, 50, 55, 60, 65, 70, 75, 80, 85, 90, 95,100,105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 nucleotides.

In some embodiments, an engineered nucleic acid herein can comprise a nucleic acid sequence from Table 5. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 47-78 or 427-432. In some embodiments, an engineered nucleic acid herein can comprise a nucleic acid sequence from Table 6. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 79-174. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 331-426. In some embodiments, an engineered nucleic acid herein can comprise a nucleic acid sequence from Table 7. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 175-252. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 214-252. In some embodiments, an engineered nucleic acid herein can comprise a nucleic acid sequence from Table 8. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 253-330. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 292-330.

In some embodiments, an engineered nucleic acid herein can comprise a nucleic acid sequence from Table 5. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NOs: 47-78 or 427-432. In some embodiments, an engineered nucleic acid herein can comprise a nucleic acid sequence from Table 6. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NOs: 79-174. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NOs: 331-426. In some embodiments, an engineered nucleic acid herein can comprise a nucleic acid sequence from Table 7. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NOs: 175-252. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NOs: 214-252. In some embodiments, an engineered nucleic acid herein can comprise a nucleic acid sequence from Table 8. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NOs: 253-330. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NOs: 292-330.

In some embodiments, a nucleic acid herein can comprise a nuclear localization sequence. In some embodiments, a nucleic acid herein can comprise a chemical modification. In some cases, a chemical modification can comprise a phosphodiester modification, methylation of a hydroxyl group, an epigenetically marked base, a deoxyribose sugar, or a combination thereof. In some cases, a chemical modification can comprise a phosphodiester modification. In some cases, a chemical modification can comprise a methylation of a hydroxyl group. In some cases, a chemical modification can comprise an epigenetically marked base. In some cases, a chemical modification can comprise a deoxyribose sugar.

In some embodiments, an engineered nucleic acid herein can comprise a nucleic acid sequence from Table 9 or Table 10. Table 9 shows different miR-132 targeting engineered sequences. Table 10 shows different miR-155 targeting engineered sequences. Table 9 and Table 10 show design descriptions of the engineered RNA, the SEQ ID NO, and the sequence of the engineered RNA. In the design description of the sequences 1×-6× sites indicate the number of binding sites (e.g., x1 is equal to one binding site while x6 is equal to six binding sites); x nt refers to the length of the linker sequence separating each stem loop; ND is no base deletion in the arms; BD is a single nt base deletion in one stem loop arm; MM is a mismatch in the stem loop arm; Perf A/G/C/U refer to an imperfect seed by inserting either A/G/C/U at position 6 from the 3′ end of the binding site; Perf pos x (e.g., pos2) refers to an imperfect seed with a mismatch at position x from the 3′end of the binding site; Imperf A/G/C/U refer to having one deletion and two mismatches at positions 9-11 and having an imperfect seed by inserting either A/G/C/U at position 6 from the 3′ end of the binding site; Imperf pos x (e.g. pos3) refer to having one deletion and two mismatches at positions 9-11 and an imperfect seed with a mismatch at position x from the 3′end of the binding site; x/X aux arms (e.g., 4/4) refers to the auxiliary arm length, for example a 4/4/is a 4 nt pair auxiliary arm size length. The RNA sequence of any sequence in Table 9 or Table 10 can be converted to a DNA sequence by substituting any “U” with a “T”.

TABLE 9
miR-132 engineered RNA sequences

| Design | SEQ ID | |
|---|---|---|
| Description | NO: | Sequence |
| x1 site | 433 | gggUGAUAUACGACCAGUauagacuacaaCGACCAUGGCUCAGACUGUUAgaaacucaagauACUGGUCUAUAUCAuuaugagc |
| x2 site | 434 | gggUGAUAUACGACCAGUauagacuacaaCGACCAUGGCUCAGACUGUUAgaaacucaagauACUGGUCUAUAUCAuuaugagcU |
| | | GAUAAGAAAUGACUcaacuuacguaCGACCAUGGCUCAGACUGUUAcaauaucaccucAGUCAUUCUUAUCAcuuagaca |
| x3 site | 435 | gggUGAUAUACGACCAGUauagacuacaaCGACCAUGGCUCAGACUGUUAgaaacucaagauACUGGUCUAUAUCAuuaugagcU |
| | | GAUAAGAAAUGACUcaacuuacguaCGACCAUGGCUCAGACUGUUAcaauaucaccucAGUCAUUCUUAUCAcuuagacaCAUUA |
| | | GUAUGUUAAGcucauccuuauCGACCAUGGCUCAGACUGUUAcacucaaaucauCUUAACAACUAAUGuuuaguag |
| x4 site | 436 | gggUGAUAUACGACCAGUauagacuacaaCGACCAUGGCUCAGACUGUUAgaaacucaagauACUGGUCUAUAUCAuuaugagcU |
| | | GAUAAGAAAUGACUcaacuuacguaCGACCAUGGCUCAGACUGUUAcaauaucaccucAGUCAUUCUUAUCAcuuagacaCAUUA |
| | | GUAUGUUAAGcucauccuuauCGACCAUGGCUCAGACUGUUAcacucaaaucauCUUAACAACUAAUGuuuaguagUCAAUACA |
| | | GAUUAUGacucuaucaaaCGACCAUGGCUCAGACUGUUAgcaacaucaucaCAUAAUCGUAUUGAcuugcaau |
| x5 site | 437 | gggUGAUAUACGACCAGUauagacuacaaCGACCAUGGCUCAGACUGUUAgaaacucaagauACUGGUCUAUAUCAuuaugagcU |
| | | GAUAAGAAAUGACUcaacuuacguaCGACCAUGGCUCAGACUGUUAcaauaucaccucAGUCAUUCUUAUCAcuuagacaCAUUA |
| | | GUAUGUUAAGcucauccuuauCGACCAUGGCUCAGACUGUUAcacucaaaucauCUUAACAACUAAUGuuuaguagUCAAUACA |
| | | GAUUAUGacucuaucaaaCGACCAUGGCUCAGACUGUUAgcaacaucaucaCAUAAUCGUAUUGAcuugcaauCUAUAUACUUC |
| | | AAGGaagaucacuaaCGACCAUGGCUCAGACUGUUAcacuacuaccaaCCUUGAAUAUAUAGccguaguu |
| x6 site | 438 | gggUGAUAUACGACCAGUauagacuacaaCGACCAUGGCUCAGACUGUUAgaaacucaagauACUGGUCUAUAUCAuuaugagcU |
| | | GAUAAGAAAUGACUcaacuuacguaCGACCAUGGCUCAGACUGUUAcaauaucaccucAGUCAUUCUUAUCAcuuagacaCAUUA |
| | | GUAUGUUAAGcucauccuuauCGACCAUGGCUCAGACUGUUAcacucaaaucauCUUAACAACUAAUGuuuaguagUCAAUACA |
| | | GAUUAUGacucuaucaaaCGACCAUGGCUCAGACUGUUAgcaacaucaucaCAUAAUCGUAUUGAcuugcaauCUAUAUACUUC |
| | | AAGGaagaucacuaaCGACCAUGGCUCAGACUGUUAcacuacuaccaaCCUUGAAUAUAUAGccguaguuCUGUUUAUUGACAU |
| | | GucaacaucaauCGACCAUGGCUCAGACUGUUAcacuuuaccuaaCAUGUCAUAAACAGuaacagaa |
| x3 sites- | 439 | gggugauauacgaccaguAUUACAUUAUAcgaccauggcucagacuguuaCUUACCCACUCAacuggucuauaucaUGugauaagaaaugac |
| 2n t | | uCUCCACUUUCCcgaccauggcucagacuguuaAAACUCUUAUAAagucauucuuaucaAAcauuaguauguuaagUAAAUUCAUUAcg |
| linker | | accauggcucagacuguuaCCAAUUCACCUCcuuaacaacuaaugCC |
| x3 sites- | 440 | gggugauauacgaccaguAUUUACAUAUUcgaccauggcucagacuguuaCUAAUUCACAAAacuggucuauaucaUAAUugauaagaaau |
| 4 nt | | gacuAUACCAAAUCUcgaccauggcucagacuguuaAAAUCAUACCAAagucauucuuaucaCAUUcauuaguauguuaagCUCACACCU |
| linker | | CUcgaccauggcucagacuguuaAAAUCCUAAUCAcuuaacaacuaaugUAUU |
| x3 sites- | 441 | gggugauauacgaccaguUCCUUUAUACAcgaccauggcucagacuguuaACAUAUUCUUUCacuggucuauaucaUCAUUUugauaaga |
| 6 nt | | aaugacuCACGAACACCCcgaccauggcucagacuguuaCCUACUCCUAUUagucauucuuaucaUCCACAcauuaguauguuaagUAUU |
| linker | | CCCAACCcgaccauggcucagacuguuaACACCAAUUUCCcuuaacaacuaaugCUUCUC |
| x3 sites- | 442 | gggugauauacgaccaguUUCUCACACUUcgaccauggcucagacuguuaAUACCAAUCUAUacuggucuauaucaCUUAUACUugauaa |
| 8 nt | | gaaaugacuCUCCACUUAAAcgaccauggcucagacuguuaCAACUAUCCUUUagucauucuuaucaUACCUAUCcauuaguauguuaag |
| linker | | UAAACCUACUUcgaccauggcucagacuguuaACUCCUUCUCAUcuuaacaacuaaugAACAAACU |
| x6 sites- | 443 | gggugauauacgaccaguAUUACAUUAUAcgaccauggcucagacuguuaCUUACCCACUCAacuggucuauaucaUGugauaagaaaugac |
| 2 nt | | uCUCCACUUUCCcgaccauggcucagacuguuaAAACUCUUAUAAagucauucuuaucaAAcauuaguauguuaagUAAAUUCAUUAcg |
| linker | | accauggcucagacuguuaCCAAUUCACCUCcuuaacaacuaaugCCucaauacagauuaugUAUCUCACGUAcgaccauggcucagacugu |
| | | uaAUAACUCCACCUcauaaucguauugaCUcuauauacuucaaggCUACCAAACAAcgaccauggcucagacuguuaACAAUUACCACAc |
| | | cuugaauauauagCCcuguuuauugacaugACCCUCCGCAAcgaccauggcucagacuguuaCCACCUUACUCAcaugucauaaacagCC |
| x6 sites- | 444 | gggugauauacgaccaguAUUUACAUAUUcgaccauggcucagacuguuaCUAAUUCACAAAacuggucuauaucaUAAUugauaagaaau |
| 4 nt | | gacuAUACCAAAUCUcgaccauggcucagacuguuaAAAUCAUACCAAagucauucuuaucaCAUUcauuaguauguuaagCUCACACCU |
| linker | | CUcgaccauggcucagacuguuaAAAUCCUAAUCAcuuaacaacuaaugUAUUucaauacagauuaugCAAAUUAAGAAcgaccauggcuc |
| | | agacuguuaCAAUACCACGCAcauaaucguauugaUCCUcuauauacuucaaggUAAUCCCUAUCcgaccauggcucagacuguuaCUUA |
| | | UCCACUUCccuugaauauauagCCUAcuguuuauugacaugCCCUCCACCGAcgaccauggcucagacuguuaCCAACCCUAUCAcauguc |
| | | auaaacagAUAA |
| x6 sites- | 445 | gggugauauacgaccaguUCCUUUAUACAcgaccauggcucagacuguuaACAUAUUCUUUCacuggucuauaucaUCAUUUugauaaga |
| 6 nt | | aaugacuCACGAACACCCcgaccauggcucagacuguuaCCUACUCCUAUUagucauucuuaucaUCCACAcauuaguauguuaagUAUU |
| linker | | CCCAACCcgaccauggcucagacuguuaACACCAAUUUCCcuuaacaacuaaugCUUCUCucaauacagauuaugUACUAUAUGCUcgac |
| | | cauggcucagacuguuaCACUUACCAAACcauaaucguauugaCCAACAcuauauacuucaaggCGAAUAACAAAcgaccauggcucagacu |
| | | guuaAACCUUUACAAUccuugaauauauagCUAUCAcuguuuauugacaugCUUAACCCGAAcgaccauggcucagacuguuaCAACCA |
| | | AUACUAcaugucauaaacagCACCCG |
| x6 sites- | 446 | gggugauauacgaccaguUUCUCACACUUcgaccauggcucagacuguuaAUACCAAUCUAUacuggucuauaucaCUUAUACUugauaa |
| 8 nt | | gaaaugacuCUCCACUUAAAcgaccauggcucagacuguuaCAACUAUCCUUUagucauucuuaucaUACCUAUCcauuaguauguuaag |
| linker | | UAAACCUACUUcgaccauggcucagacuguuaACUCCUUCUCAUcuuaacaacuaaugAACAAACUucaauacagauuaugCAACGCAA |
| | | ACCcgaccauggcucagacuguuaAACCAAUACACCcauaaucguauugaCGAAACUAcuauauacuucaaggCUCUUCACACCcgaccau |
| | | ggcucagacuguuaAAAUCCUCUAUCccuugaauauauagAACCUCUAcuguuuauugacaugCAAUAUUUAAUcgaccauggcucagac |
| | | uguuaAACUUCCAACACcaugucauaaacagCGCGCCUC |
| x3 sites- | 447 | gggucaauacagauuUCACUUUCACUcgaccauggcucagacuguuaCAUAACCUAAUCaaucuguauugaAACAUUACcuauauacuuc |
| 12 nt | | aGCUCCACAUAUcgaccauggcucagacuguuaCCCUCCAAUCAAugaaguauauagCCAUUCACcuguuuauugacCACCUUCAACAc |
| ND | | gaccauggcucagacuguuaCCUCAUAUGCACgucaauaaacagUUCACCUG |
| x3 sites- | 448 | gggUCUGCAUUGCUAcccaaccaacaCGACCAUGGCUCAGACUGUUAacuuuccucaauUAGCAAGCAGAcaaacucaCUCUUCGA |
| 12 nt | | ACCAucacuaaauucCGACCAUGGCUCAGACUGUUAcauaucacuauuUGGUUCAAGAGaacuccaaGCCACAGCAACAgcguauu |
| BD | | uaacCGACCAUGGCUCAGACUGUUAcuuuccucauaaUGUUGCGUGGCcccaauua |
| x3 sites- | 449 | gggCCGAUACCAUCAaugcaacuucaCGACCAUGGCUCAGACUGUUAaacacaacuauaUGAUGGAAUCGGuaccuaacUCUACU |
| 12 nt | | UCAGAGcacuauacaaaCGACCAUGGCUCAGACUGUUAaacauuuaccacCUCUGAUGUAGAucauacagGCAUAUACAACGcucc |
| MM | | aaccgaaCGACCAUGGCUCAGACUGUUAccuaaccuacacCGUUGUUUAUGCgaaucuca |
| x3 sites- | 450 | gggugauauacgaccaguUUAUUCUCCGAcgaccauggcucagacuguuaAGACUCCUACCCacuggucguauaucaCCCGUCUUugauaa |
| 15 nt | | gaaaugacuCCCUUCCUUAUcgaccauggcucagacuguuaCUAUACCUUUCCagucauuucuuaucaCAACUUUAcauuaguauguuaa |
| ND | | gUGCACCUAAUUcgaccauggcucagacuguuaACACUACCUAUUcuuaacauacuaaugAUUUACAA |
| x3 sites- | 451 | gggugauauacgaccaguAAACCAACCACcgaccauggcucagacuguuaCAAUUUCCAUACacuggucuauaucaUAAUUUAUugauaag |
| 15 nt | | aaaugacuCUCCUUCUACUcgaccauggcucagacuguuaACCACUUCUAUUagucauucuuaucaUUCUAACCcauuaguauguuaagU |
| BD | | CAUACCAACCcgaccauggcucagacuguuaCCUAUAUUUACUcuuaacaacuaaugACUUCACU |
| x3 sites- | 452 | gggugauauacgaccaguUAUACAUUAAUcgaccauggcucagacuguuaAACACUACCCACacugguccuauaucaUCAUAUAAugauaa |
| 15 nt | | gaaaugacuCUUUCCUCAACcgaccauggcucagacuguuaCACCUAUUCAUAagucauuacuuaucaCACCUAUUcauuaguauguuaa |
| MM | | gUAUUCUUAUCAcgaccauggcucagacuguuaCUAAAUCUAACCcuuaacaaacuaaugCACUAUGC |
| x6 sites- | 453 | ggguauacgaccaguCAACAUAACGAcgaccauggcucagacuguuaGCACCUCCAUAAacuggucguauaUAAUU |
| 12 nt | | GCCugauaagaaaugCUCUAUAAUCAcgaccauggcucagacuguuaAUUAAUCUCUAAcauuucuuaucaCCCU |
| ND | | ACGAcauuaguauguuUCUCUUUCUAAcgaccauggcucagacuguuaCUAACACAUAUUaacauacuaaugCCU |
| | | UAAUAucaauacagauuUCACUUUCACUcgaccauggcucagacuguuaCAUAACCUAAUCaaucuguauugaAA |
| | | CAUUACcuauauacuucaGCUCCACAUAUcgaccauggcucagacuguuaCCCUCCAAUCAAugaaguauauagCC |
| | | AUUCACcuguuuauugacCACCUUCAACAcgaccauggcucagacuguuaCCUCAUAUGCACgucaauaaacagUU |
| | | CACCUG |
| x6 sites- | 454 | gggGUCAGAUACAUCCCUAUACGAAACGACCAUGGCUCAGACUGUUAAUUUAAUGCACCGAUGUACUGACCCCUAUAACC |
| 12 nt | | AGCUCUUGCAUUUCCAAUACGCGACCAUGGCUCAGACUGUUACAACCCUCCAUUUGCAAGGCUGGCCCUCAACUGCCAA |
| BD | | AUGCCUACCUAUACCUUCGACCAUGGCUCAGACUGUUAAUAAUUCCCUCGAGGCAUUGGCACUUUCUCACCGUAUAGCC |
| | | CUUACACAAAUCUCGACCAUGGCUCAGACUGUUAAAUCUCUAUCACAGGGCUUACGGGUCCCGAACUUUGUCAGCAUCC |
| | | ACACCUUAUCGACCAUGGCUCAGACUGUUAAAUUAAUAUUUCAUGCUGCAAAGAUUACCGCUCAGCACUCUUCACUAAU |
| | | CCUAUCGACCAUGGCUCAGACUGUUAACAUCACCGCACGAAGAGGCUGAUCUUAAAG |
| x6 sites- | 455 | gggUCUCGUCACUCUUUCCUCUCCUACGACCAUGGCUCAGACUGUUAACCACUUAUCUUAGAGUGUCGAGAAACCAUAC |
| 12 nt | | ACCUCUACCGCAAACUUACUUCACGACCAUGGCUCAGACUGUUAACCUUUCUUAUCUGCGGUUGAGGUUACUUGACUC |
| MM | | ACGUUCUUGCCCAAUACUCCCCGACCAUGGCUCAGACUGUUACCCAAUUCCACUGCAAGAUCGUGACUAUCCAAAAGCAC |
| | | CAGAUCCAUUACAAAUACGACCAUGGCUCAGACUGUUAACAAUACUGCAAGAUCUGCUGCUUCAAAUACCUAAAUAUGC |
| | | UUCGAUAAACUACACGACCAUGGCUCAGACUGUUACCCUUCAUUGCAGAAGCAAAUUUAAUCUUAACUCACAAGCUCAA |
| | | GACCGAUACCACGACCAUGGCUCAGACUGUUACAUCCUUACUAAUUGAGCAUGUGAAAUCCCUU |
| x6 sites- | 585 | gggugauauacgaccaguUUAUUCUCCGAcgaccauggcucagacuguuaAGACUCCUACCCacuggucguauaucaCCCGUCUUugauaa |
| 15 nt | | gaaaugacuCCCUUCCUUAUcgaccauggcucagacuguuaCUAUACCUUUCCagucauuucuuaucaCAACUUUAcauuaguauguuaa |
| ND | | gUGCACCUAAUUcgaccauggcucagacuguuaACACUACCUAUUcuuaacauacuaaugAUUUACAAucaauacagauuaugUUCUCA |
| | | UUAUAcgaccauggcucagacuguuaCAAAUCUCAUCUcauaaucuguauugaAUUCUGUCcuauauacuucaaggAUCUAUCACGAcg |
| | | accauggcucagacuguuaCAUACAUAACUAccuugaaguauauagAAACUCCUcuguuuauugacaugUCUAAACGCACcgaccauggcu |
| | | cagacuguuaCUAUUCCUCUCCcaugucaauaaacagUCACCAAA |
| x6 sites- | 456 | gggugauauacgaccaguAAACCAACCACcgaccauggcucagacuguuaCAAUUUCCAUACacuggucuauaucaUAAUUUAUugauaag |
| 15 nt | | aaaugacuCUCCUUCUACUcgaccauggcucagacuguuaACCACUUCUAUUagucauucuuaucaUUCUAACCcauuaguauguuaagU |
| BD | | CAUACCAACCcgaccauggcucagacuguuaCCUAUAUUUACUcuuaacaacuaaugACUUCACUucaauacagauuaugCACCUAAAU |
| | | CAcgaccauggcucagacuguuaCUUAUACACUUCcauaaucguauugaCUAACUUCcuauauacuucaaggCGACUACACUAcgaccaug |
| | | gcucagacuguuaCCACACGAUUUAccuugaauauauagACACCUGAcuguuuauugacaugAAUCUACAAUAcgaccauggcucagacug |
| | | uuaCCAACCACACCAcaugucauaaacagCCUUUCUG |
| x6 sites- | 457 | gggugauauacgaccaguUAUACAUUAAUcgaccauggcucagacuguuaAACACUACCCACacugguccuauaucaUCAUAUAAugauaa |
| 15 nt | | gaaaugacuCUUUCCUCAACcgaccauggcucagacuguuaCACCUAUUCAUAagucauuacuuaucaCACCUAUUcauuaguauguuaa |
| MM | | gUAUUCUUAUCAcgaccauggcucagacuguuaCUAAAUCUAACCcuuaacaaacuaaugCACUAUGCucaauacagauuaugCAUUCA |
| | | AAUCAcgaccauggcucagacuguuaAAGAAAUUACAAcauaaucaguauugaCUACAUUCcuauauacuucaaggAAUCUUUCUCAcg |
| | | accauggcucagacuguuaACUUACCUUUCAccuugaacuauauagCUAACCAAcuguuuauugacaugAUCUAUACGCAcgaccauggcu |
| | | cagacuguuaCAUUAUCACCUAcaugucauuaaacagAAUUCUGU |
| x3 sites- | 458 | gggugauauacgaccaguUAUCAAAUUAACGACCAUGGCUGUAGACUGUUAGACAAUCAAAUCacuggucuauaucaCCAAACUU |
| Perf | | ugauaagaaaugacuUUACUAAUCAACGACCAUGGCUGUAGACUGUUAGACACAUAUUCUagucauucuuaucaCCGAAUACca |
| ctrl | | uuaguauguuaagUAUAAACCGAACGACCAUGGCUGUAGACUGUUAAACCUCUCAAAUcuuaacaacuaaugAUGCAACU |
| x3 sites- | 459 | gggugauauacgaccaguUCACACCGUACCGACCAUGGCUGUAGACaUGUUAACUGACAAUUAUacuggucuauaucaUCGACUC |
| Perf A | | GugauaagaaaugacuGUUCAAUGUACCGACCAUGGCUGUAGACaUGUUACCACCGCUUUAGagucauucuuaucaGCUCAAUC |
| | | cauuaguauguuaagCUUUCAAUAUCCGACCAUGGCUGUAGACaUGUUAUUAUUCCGUAUUcuuaacaacuaaugAUUUCCUA |
| x3 sites- | 460 | gggugauauacgaccaguUACGCACCUUUCGACCAUGGCUGUAGACgUGUUAGUUCCGAACAUUacuggucuauaucaCCGACUC |
| Perf G | | CugauaagaaaugacuCCUUUCCUUAACGACCAUGGCUGUAGACgUGUUAUGCCGCUUAUUCagucauucuuaucaACUUCACC |
| | | cauuaguauguuaagAAUAACCCGCCCGACCAUGGCUGUAGACgUGUUAUGACAAUUUCAGcuuaacaacuaaugAUUAUCCA |
| x3 sites- | 461 | gggugauauacgaccaguUCCCACGAUCUCGACCAUGGCUGUAGACCUGUUAAUCUGAACUCCUacuggucuauaucaCCAACGA |
| Perf C | | CugauaagaaaugacuGAAUCACAAUGCGACCAUGGCUGUAGACCUGUUAUAAACCUCUUAAagucauucuuaucaCACCCAACc |
| | | auuaguauguuaagAACCAUAUCCCCGACCAUGGCUGUAGACCUGUUACCUCAAUUAUCGcuuaacaacuaaugACAACGCG |
| x3 sites- | 462 | gggugauauacgaccaguCCUAUUUAAUUCGACCAUGGCUGUAGACuUGUUAAACCCUUCCAACacuggucuauaucaACCCUAU |
| Perf U | | CugauaagaaaugacuAUCCACUAACACGACCAUGGCUGUAGACuUGUUAAUAUUACUUAUCagucauucuuaucaACGCCGACc |
| | | auuaguauguuaagUCCAAAUAACCCGACCAUGGCUGUAGACuUGUUAAACCCUCCGCUUcuuaacaacuaaugAUUACUUA |
| x3 sites- | 463 | gggugauauacgaccaguACUAAGAAUAUcgaccauggcuguagacuguaaUACUUAAUUUAAacuggucuauaucauuaugagcugauaa |
| Perf | | gaaaugacuAUAAAUUAUCAcgaccauggcuguagacuguaaCUAAUCAACUAAagucauucuuaucauucagacacauuaguauguuaag |
| pos2 | | ACUAAUUAUCUcgaccauggcuguagacuguaaCUAAUCCUUCACcuuaacaacuaauguuuaguag |
| x3 sites- | 464 | gggugauauacgaccaguAGAGAACAUAAcgaccauggcuguagacugauaAUAACGUACCGAacuggucuauaucauuaugagcugauaag |
| Perf | | aaaugacuGUAUAAUUUCCcgaccauggcuguagacugauaACUUACCACAUAagucauucuuaucauucagacacauuaguauguuaagU |
| pos3 | | UCCAACUCUUcgaccauggcuguagacugauaAUCCGACUCUUCcuuaacaacuaauguuuaguag |
| x3 sites- | 465 | gggugauauacgaccaguAACACAUUAAUcgaccauggcuguagacucuuaUCCUUACUUAAAacuggucuauaucauuaugagcugauaag |
| Perf | | aaaugacuUCAAAGUAUAUcgaccauggcuguagacucuuaAUCCCUAAUCCUagucauucuuaucauucagacacauuaguauguuaagC |
| pos4 | | UUAUCCUAGAcgaccauggcuguagacucuuaAUAUCAAAUCAAcuuaacaacuaauguuuaguag |
| x3 sites- | 466 | gggugauauacgaccaguACACAACGUUCcgaccauggcuguagacaguuaAUAUAAUUCAAGacuggucuauaucauuaugagcugauaag |
| Perf | | aaaugacuUGAACACCUACcgaccauggcuguagacaguuaAACCUUUAUAAUagucauucuuaucauucagacacauuaguauguuaagA |
| pos5 | | AGACAUUUACcgaccauggcuguagacaguuaAACAAUCACAUAcuuaacaacuaauguuuaguag |
| x3 sites- | 467 | gggugauauacgaccaguCAUCAACUAAUcgaccauggcuguagaguguuaAUAUCUCAAUUCacuggucuauaucauuaugagcugauaag |
| Perf | | aaaugacuCUCCCUCACGAcgaccauggcuguagaguguuaGAUCAAUUCAUAagucauucuuaucauucagacacauuaguauguuaagU |
| pos6 | | UAAUUCAAACcgaccauggcuguagaguguuaCCUCCCUCAAUUcuuaacaacuaauguuuaguag |
| x3 sites- | 468 | gggugauauacgaccaguGAUAUAACUUUcgaccauggcuguagucuguuaACACCCGCUCAAacuggucuauaucauuaugagcugauaag |
| Perf | | aaaugacuUUCAAUAAGUAcgaccauggcuguagucuguuaUUCACACCCUCUagucauucuuaucauucagacacauuaguauguuaagC |
| pos7 | | UCCACCGUCGcgaccauggcuguagucuguuaUCAUAUGUCUUUcuuaacaacuaauguuuaguag |
| x3 sites- | 469 | gggugauauacgaccaguCAUUAAUCCUCcgaccauggcuguacacuguuaUUACCAAACACUacuggucuauaucauuaugagcugauaag |
| Perf | | aaaugacuCUCUCGCUUAUcgaccauggcuguacacuguuaCCUAACCUAAUCagucauucuuaucauucagacacauuaguauguuaagU |
| pos8 | | CCUAAUAUCGcgaccauggcuguacacuguuaAUAAAGUCAAUUcuuaacaacuaauguuuaguag |
| x3 sites- | 435 | gggUGAUAUACGACCAGUauagacuacaaCGACCAUGGCUCAGACUGUUAgaaacucaagauACUGGUCUAUAUCAuuaugagcU |
| Imperf | | GAUAAGAAAUGACUcaacuuacguaCGACCAUGGCUCAGACUGUUAcaauaucaccucAGUCAUUCUUAUCAcuuagacaCAUUA |
| ctrl | | GUAUGUUAAGcucauccuuauCGACCAUGGCUCAGACUGUUAcacucaaaucauCUUAACAACUAAUGuuuaguag |
| x3 sites- | 470 | gggugauauacgaccaguGUGCUCCACCUCGACCAUGGCUCAGACaUGUUACAUACUUUGAUGacuggucuauaucaCCACAAUU |
| Imperf | | ugauaagaaaugacuAUUAAGAUUACCGACCAUGGCUCAGACaUGUUAAAAUAGAUAUCGagucauucuuaucaCCAGAUCCcau |
| A | | uaguauguuaagAAACCGUUCUGCGACCAUGGCUCAGACaUGUUAAAAUAAGAUAUGcuuaacaacuaaugCAGUAGCA |
| x3 sites- | 471 | gggugauauacgaccaguUCCUCAGUAAACGACCAUGGCUCAGACgUGUUAUAUUCUAAUCCCacuggucuauaucaUUCACACU |
| Imperf | | ugauaagaaaugacuAGAAAUUGACUCGACCAUGGCUCAGACgUGUUAAAAUAUUGAAUAagucauucuuaucaUAAAUCCAca |
| G | | uuaguauguuaagAAUGAACCCACCGACCAUGGCUCAGACgUGUUAAAAUAUUGUGAAcuuaacaacuaaugGAAUGACU |
| x3 sites- | 472 | gggugauauacgaccaguUCACUCUUAAACGACCAUGGCUCAGACCUGUUAAAACCUUACAUUacuggucuauaucaAUAAUGUU |
| Imperf | | ugauaagaaaugacuUGAUAUCUACCCGACCAUGGCUCAGACCUGUUAAAAUGCUCCUAUagucauucuuaucaUUUACUCUca |
| C | | uuaguauguuaagAGACUAGAAUACGACCAUGGCUCAGACCUGUUAAAAUAUCCACAAcuuaacaacuaaugUAUGAACC |
| x3 sites- | 473 | gggugauauacgaccaguUGCACGCACUCCGACCAUGGCUCAGACuUGUUAAAAUUAUACACUacuggucuauaucaCCCUUACU |
| Imperf | | ugauaagaaaugacuCUAAAUGCACUCGACCAUGGCUCAGACuUGUUAAAUAUUCUACACagucauucuuaucaUUCGUCUCcau |
| U | | uaguauguuaagAUCUCUAUCUUCGACCAUGGCUCAGACUUGUUACACUUAAAUUCGcuuaacaacuaaugUCUCAACC |
| x3 sites- | 474 | gggugauauacgaccaguCAACCAACUAUcgaccauggcucagacuguaaCAUUUAACUCAAacuggucuauaucaCCACUCCCugauaag |
| Imperf | | aaaugacuAUAUACUAAUUcgaccauggcucagacuguaaUAUAAGACAUCAagucauucuuaucaAAACUAUCcauuaguauguuaag |
| pos2 | | UGACACGCACCcgaccauggcucagacuguaaAUUCCAAAUCCUcuuaacaacuaaugCAUUCCAA |
| x3 sites- | 475 | gggugauauacgaccaguAAUACACUUCAcgaccauggcucagacugauaUUACUAUAAACCacuggucuauaucaCUACAAUAugauaag |
| Imperf | | aaaugacuUUUCCACACUAcgaccauggcucagacugauaUAAAUCUAACAUagucauucuuaucaAGCAACCAcauuaguauguuaagA |
| pos3 | | UACAUAAUUAcgaccauggcucagacugauaACCUUCCUUCCCcuuaacaacuaaugCUUUAAUA |
| x3 sites- | 476 | gggugauauacgaccaguUCAGACAAACAcgaccauggcucagacucuuaCUAUCUCAACACacuggucuauaucaCAUUUACAugauaag |
| Imperf | | aaaugacuUGAACAAACGAcgaccauggcucagacucuuaCUAAAUUCUCUUagucauucuuaucaCAUCUACUcauuaguauguuaagG |
| pos4 | | AACAGCAUUCcgaccauggcucagacucuuaGAUACAGUACCAcuuaacaacuaaugCAUCAAUA |
| x3 sites- | 477 | gggugauauacgaccaguUAUACAGACUAcgaccauggcucagacaguuaCAAACGCGAACCacuggucuauaucaCACAAUUUugauaag |
| Imperf | | aaaugacuUUACAAAUAUCcgaccauggcucagacaguuaAAACCGCAACAUagucauucuuaucaUCCCGCAUcauuaguauguuaagC |
| pos5 | | UACAAAUCACcgaccauggcucagacaguuaGACACCUUCCCUcuuaacaacuaaugUUCUACAC |
| x3 sites- | 478 | gggugauauacgaccaguUUUCUAUCCUUcgaccauggcucagaguguuaAUACUAUAUCAUacuggucuauaucaUCUAUCUUugaua |
| Imperf | | agaaaugacuAUCCUACCCGAcgaccauggcucagaguguuaUAUCCAUUGACGagucauucuuaucaUUACCAACcauuaguauguuaa |
| pos6 | | gAAAUCUUCCACcgaccauggcucagaguguuaAACCAUCUACAGcuuaacaacuaaugUGCUCAUC |
| x3 sites- | 479 | gggugauauacgaccaguCUGCGAAUACCcgaccauggcucagucuguuaCCUUUGAUCUUAacuggucuauaucaUCUACCUCugauaa |
| Imperf | | gaaaugacuGUGAACAUUCCcgaccauggcucagucuguuaUCUAACGAAAUAagucauucuuaucaAACUCAACcauuaguauguuaag |
| pos7 | | AGAUACUUACUcgaccauggcucagucuguuaUCCUACCUUCCGcuuaacaacuaaugUACUCAUC |
| x3 sites- | 480 | gggugauauacgaccaguACCCUCAUCUAcgaccauggcucacacuguuaUUCUUAUCAUUAacuggucuauaucaUUCCAAAUugauaa |
| Imperf | | gaaaugacuCUGACUAUUCGcgaccauggcucacacuguuaAAUAUGAAACCUagucauucuuaucaCACUCAACcauuaguauguuaag |
| pos8 | | GCAAUCUACUUcgaccauggcucacacuguuaUACAACCUACGAcuuaacaacuaaugAAGCUCAG |
| x6 sites- | 481 | gggugauauacgaccaguUAUCAAAUUAACGACCAUGGCUGUAGACUGUUAGACAAUCAAAUCacuggucuauaucaCCAAACUU |
| Perf | | ugauaagaaaugacuUUACUAAUCAACGACCAUGGCUGUAGACUGUUAGACACAUAUUCUagucauucuuaucaCCGAAUACca |
| ctrl | | uuaguauguuaagUAUAAACCGAACGACCAUGGCUGUAGACUGUUAAACCUCUCAAAUcuuaacaacuaaugAUGCAACUucaa |
| | | uacagauuaugAUACAAAUUCACGACCAUGGCUGUAGACUGUUAGAUUACCUUUAGcauaaucguauugaCUCUGAACcuauau |
| | | acuucaaggCUCUAAUCUUUCGACCAUGGCUGUAGACUGUUAAAAUGUACCAAUccuugaauauauagAAGAUUUAcuguuuau |
| | | ugacaugUACUCAUAAUCCGACCAUGGCUGUAGACUGUUAAUAACCCAACCUcaugucauaaacagAAACAUAA |
| x6 sites- | 482 | gggugauauacgaccaguUCACACCGUACCGACCAUGGCUGUAGACaUGUUAACUGACAAUUAUacuggucuauaucaUCGACUC |
| Perf A | | GugauaagaaaugacuGUUCAAUGUACCGACCAUGGCUGUAGACaUGUUACCACCGCUUUAGagucauucuuaucaGCUCAAUC |
| | | cauuaguauguuaagCUUUCAAUAUCCGACCAUGGCUGUAGACaUGUUAUUAUUCCGUAUUcuuaacaacuaaugAUUUCCUA |
| | | ucaauacagauuaugAGAGUCCCAUACGACCAUGGCUGUAGACaUGUUAAGACUAAUCAAGcauaaucguauugaAUCUGAACc |
| | | uauauacuucaaggUCCACGCUCCACGACCAUGGCUGUAGACaUGUUAACUGAAUCAUACccuugaauauauagCUGCCACGcug |
| | | uuuauugacaugCUAAUUAACCCCGACCAUGGCUGUAGACaUGUUAAGCAAUACAUAUcaugucauaaacagAUAUUAAG |
| x6 sites- | 483 | gggugauauacgaccaguUACGCACCUUUCGACCAUGGCUGUAGACgUGUUAGUUCCGAACAUUacuggucuauaucaCCGACUC |
| Perf G | | CugauaagaaaugacuCCUUUCCUUAACGACCAUGGCUGUAGACgUGUUAUGCCGCUUAUUCagucauucuuaucaACUUCACC |
| | | cauuaguauguuaagAAUAACCCGCCCGACCAUGGCUGUAGACgUGUUAUGACAAUUUCAGcuuaacaacuaaugAUUAUCCAu |
| | | caauacagauuaugAGAACAUCCCUCGACCAUGGCUGUAGACgUGUUAUUCCAACCUAACcauaaucguauugaAAUUACAAcua |
| | | uauacuucaaggGACUAUAAACUCGACCAUGGCUGUAGACgUGUUACCUAUUAAGAUAccuugaauauauagCACUAUACcugu |
| | | uuauugacaugUUGCUUUCUCCCGACCAUGGCUGUAGACgUGUUAUUCCUUGAACUCcaugucauaaacagUGCACUUU |
| x6 sites- | 484 | gggugauauacgaccaguUCCCACGAUCUCGACCAUGGCUGUAGACCUGUUAAUCUGAACUCCUacuggucuauaucaCCAACGA |
| Perf C | | CugauaagaaaugacuGAAUCACAAUGCGACCAUGGCUGUAGACCUGUUAUAAACCUCUUAAagucauucuuaucaCACCCAACc |
| | | auuaguauguuaagAACCAUAUCCCCGACCAUGGCUGUAGACCUGUUACCUCAAUUAUCGcuuaacaacuaaugACAACGCGuca |
| | | auacagauuaugACUAUCCUAGACGACCAUGGCUGUAGACCUGUUAAUUCACUCCUAAcauaaucguauugaAUCCAAUCcuaua |
| | | uacuucaaggCAUAACUAACACGACCAUGGCUGUAGACCUGUUAAUUACAAGAUUAccuugaauauauagUAGAGCACcuguuu |
| | | auugacaugAAUCCUCAACACGACCAUGGCUGUAGACCUGUUAAGACUUAACCAAcaugucauaaacagUAGACCAA |
| x6 sites- | 485 | gggugauauacgaccaguCCUAUUUAAUUCGACCAUGGCUGUAGACuUGUUAAACCCUUCCAACacuggucuauaucaACCCUAU |
| Perf U | | CugauaagaaaugacuAUCCACUAACACGACCAUGGCUGUAGACuUGUUAAUAUUACUUAUCagucauucuuaucaACGCCGACc |
| | | auuaguauguuaagUCCAAAUAACCCGACCAUGGCUGUAGACuUGUUAAACCCUCCGCUUcuuaacaacuaaugAUUACUUAuca |
| | | auacagauuaugACUCCUUCACACGACCAUGGCUGUAGACuUGUUAAACUCCUUACUGcauaaucguauugaAUGAAAUAcuau |
| | | auacuucaaggUUCAUAUUAAUCGACCAUGGCUGUAGACuUGUUACCACUCUCAAAUccuugaauauauagAUGAUCUCcuguu |
| | | uauugacaugUGCAUAAAUUCCGACCAUGGCUGUAGACuUGUUAAAUUACCAAUAUcaugucauaaacagAUUUACAA |
| x6 sites- | 486 | gggugauauacgaccaguACUAAGAAUAUcgaccauggcuguagacuguaaUACUUAAUUUAAacuggucuauaucauuaugagcugauaa |
| Perf | | gaaaugacuAUAAAUUAUCAcgaccauggcuguagacuguaaCUAAUCAACUAAagucauucuuaucauucagacacauuaguauguuaag |
| pos2 | | ACUAAUUAUCUcgaccauggcuguagacuguaaCUAAUCCUUCACcuuaacaacuaauguuuaguagucaauacagauuaugUCACUCAA |
| | | ACCcgaccauggcuguagacuguaaUUCACUUCAUAUcauaaucguauugauucuuucacuauauacuucaaggGACUUGAAACAcgacca |
| | | uggcuguagacuguaaAAAUAACUCUUAccuugaauauauaguucaugcccuguuuauugacaugUAACUUUCCUUcgaccauggcuguag |
| | | acuguaaAUCCUCACAUAUcaugucauaaacaguuauacaa |
| x6 sites- | 487 | gggugauauacgaccaguAGAGAACAUAAcgaccauggcuguagacugauaAUAACGUACCGAacuggucuauaucauuaugagcugauaag |
| Perf | | aaaugacuGUAUAAUUUCCcgaccauggcuguagacugauaACUUACCACAUAagucauucuuaucauucagacacauuaguauguuaagU |
| pos3 | | UCCAACUCUUcgaccauggcuguagacugauaAUCCGACUCUUCcuuaacaacuaauguuuaguagucaauacagauuaugUGAACCUCC |
| | | GAcgaccauggcuguagacugauaAUAUCCCGCAAUcauaaucguauugauucuuucacuauauacuucaaggAUAAUUUAAUUcgaccau |
| | | ggcuguagacugauaAUCCAAUAACCCccuugaauauauaguucaugcccuguuuauugacaugAUUCCCUAGAAcgaccauggcuguagac |
| | | ugauaAAUAUUACUUUCcaugucauaaacaguuauacaa |
| x6 sites- | 488 | gggugauauacgaccaguAACACAUUAAUcgaccauggcuguagacucuuaUCCUUACUUAAAacuggucuauaucauuaugagcugauaag |
| Perf | | aaaugacuUCAAAGUAUAUcgaccauggcuguagacucuuaAUCCCUAAUCCUagucauucuuaucauucagacacauuaguauguuaagC |
| pos4 | | UUAUCCUAGAcgaccauggcuguagacucuuaAUAUCAAAUCAAcuuaacaacuaauguuuaguagucaauacagauuaugCAUUAACUU |
| | | UCcgaccauggcuguagacucuuaUCCCUCAUUAUAcauaaucguauugauucuuucacuauauacuucaaggAUUAACCGCAUcgaccaug |
| | | gcuguagacucuuaAAUAACCCGCAAccuugaauauauaguucaugcccuguuuauugacaugUAAUCCAAUUUcgaccauggcuguagacu |
| | | cuuaAUUAAUCCCUAUcaugucauaaacaguuauacaa |
| x6 sites- | 489 | gggugauauacgaccaguACACAACGUUCcgaccauggcuguagacaguuaAUAUAAUUCAAGacuggucuauaucauuaugagcugauaag |
| Perf | | aaaugacuUGAACACCUACcgaccauggcuguagacaguuaAACCUUUAUAAUagucauucuuaucauucagacacauuaguauguuaagA |
| pos5 | | AGACAUUUACcgaccauggcuguagacaguuaAACAAUCACAUAcuuaacaacuaauguuuaguagucaauacagauuaugCCCGCUCCU |
| | | AUcgaccauggcuguagacaguuaCCUAUCCUAACCcauaaucguauugauucuuucacuauauacuucaaggACAUUAUAAAUcgaccaug |
| | | gcuguagacaguuaCUUAAUCUAACAccuugaauauauaguucaugcccuguuuauugacaugCUUAAGACGAAcgaccauggcuguagaca |
| | | guuaAAUUACAACUACcaugucauaaacaguuauacaa |
| x6 sites- | 490 | gggugauauacgaccaguCAUCAACUAAUcgaccauggcuguagaguguuaAUAUCUCAAUUCacuggucuauaucauuaugagcugauaag |
| Perf | | aaaugacuCUCCCUCACGAcgaccauggcuguagaguguuaGAUCAAUUCAUAagucauucuuaucauucagacacauuaguauguuaagU |
| pos6 | | UAAUUCAAACcgaccauggcuguagaguguuaCCUCCCUCAAUUcuuaacaacuaauguuuaguagucaauacagauuaugAUAUCCCAC |
| | | AUcgaccauggcuguagaguguuaAAUCCGUAUCCCcauaaucguauugauucuuucacuauauacuucaaggCUCCCUUAACCcgaccaug |
| | | gcuguagaguguuaUAAAUUCCUAAUccuugaauauauaguucaugcccuguuuauugacaugUUCCCUUAAUCcgaccauggcuguagag |
| | | uguuaAACAAAUCUCCUcaugucauaaacaguuauacaa |
| x6 sites- | 491 | gggugauauacgaccaguGAUAUAACUUUcgaccauggcuguagucuguuaACACCCGCUCAAacuggucuauaucauuaugagcugauaag |
| Perf | | aaaugacuUUCAAUAAGUAcgaccauggcuguagucuguuaUUCACACCCUCUagucauucuuaucauucagacacauuaguauguuaagC |
| pos7 | | UCCACCGUCGcgaccauggcuguagucuguuaUCAUAUGUCUUUcuuaacaacuaauguuuaguagucaauacagauuaugUACAUUAU |
| | | CUUcgaccauggcuguagucuguuaCCAUACAUUCUCcauaaucguauugauucuuucacuauauacuucaaggCCUCAUCCUAUcgacca |
| | | uggcuguagucuguuaAUCCCGCGACAUccuugaauauauaguucaugcccuguuuauugacaugUCCAACCUUCGcgaccauggcuguagu |
| | | cuguuaUCUUUCAACACUcaugucauaaacaguuauacaa |
| x6 sites- | 492 | gggugauauacgaccaguCAUUAAUCCUCcgaccauggcuguacacuguuaUUACCAAACACUacuggucuauaucauuaugagcugauaag |
| Perf | | aaaugacuCUCUCGCUUAUcgaccauggcuguacacuguuaCCUAACCUAAUCagucauucuuaucauucagacacauuaguauguuaagU |
| pos8 | | CCUAAUAUCGcgaccauggcuguacacuguuaAUAAAGUCAAUUcuuaacaacuaauguuuaguagucaauacagauuaugACUUCACUC |
| | | AAcgaccauggcuguacacuguuaUCCUAAUCUCCGcauaaucguauugauucuuucacuauauacuucaaggCUCUUAUCUUCcgaccaug |
| | | gcuguacacuguuaAUUAAGCUUCUCccuugaauauauaguucaugcccuguuuauugacaugCAACCCUUCCAcgaccauggcuguacacu |
| | | guuaGAUACUCCGAACcaugucauaaacaguuauacaa |
| x6 sites- | 438 | gggUGAUAUACGACCAGUauagacuacaaCGACCAUGGCUCAGACUGUUAgaaacucaagauACUGGUCUAUAUCAuuaugagcU |
| Imperf | | GAUAAGAAAUGACUcaacuuacguaCGACCAUGGCUCAGACUGUUAcaauaucaccucAGUCAUUCUUAUCAcuuagacaCAUUA |
| ctrl | | GUAUGUUAAGcucauccuuauCGACCAUGGCUCAGACUGUUAcacucaaaucauCUUAACAACUAAUGuuuaguagUCAAUACA |
| | | GAUUAUGacucuaucaaaCGACCAUGGCUCAGACUGUUAgcaacaucaucaCAUAAUCGUAUUGAcuugcaauCUAUAUACUUC |
| | | AAGGaagaucacuaaCGACCAUGGCUCAGACUGUUAcacuacuaccaaCCUUGAAUAUAUAGccguaguuCUGUUUAUUGACAU |
| | | GucaacaucaauCGACCAUGGCUCAGACUGUUAcacuuuaccuaaCAUGUCAUAAACAGuaacagaa |
| x6 sites- | 493 | gggugauauacgaccaguGUGCUCCACCUCGACCAUGGCUCAGACaUGUUACAUACUUUGAUGacuggucuauaucaCCACAAUU |
| Imperf | | ugauaagaaaugacuAUUAAGAUUACCGACCAUGGCUCAGACaUGUUAAAAUAGAUAUCGagucauucuuaucaCCAGAUCCcau |
| A | | uaguauguuaagAAACCGUUCUGCGACCAUGGCUCAGACaUGUUAAAAUAAGAUAUGcuuaacaacuaaugCAGUAGCAucaau |
| | | acagauuaugCCUCAAUCCUUCGACCAUGGCUCAGACaUGUUAAAAUAUAAUGCUcauaaucguauugaAAUUUAGAcuauaua |
| | | cuucaaggAAACAGUAGAUCGACCAUGGCUCAGACaUGUUACACCAGAAAGAAccuugaauauauagCCAAGAACcuguuuauug |
| | | acaugUACUUCAAGAACGACCAUGGCUCAGACaUGUUAAAAUAAGCUUACcaugucauaaacagACCUUAUU |
| x6 sites- | 494 | gggugauauacgaccaguUCCUCAGUAAACGACCAUGGCUCAGACgUGUUAUAUUCUAAUCCCacuggucuauaucaUUCACACU |
| Imperf | | ugauaagaaaugacuAGAAAUUGACUCGACCAUGGCUCAGACgUGUUAAAAUAUUGAAUAagucauucuuaucaUAAAUCCAca |
| G | | uuaguauguuaagAAUGAACCCACCGACCAUGGCUCAGACgUGUUAAAAUAUUGUGAAcuuaacaacuaaugGAAUGACUucaa |
| | | uacagauuaugUCAACUAUCUACGACCAUGGCUCAGACgUGUUAAAAUCUUAUUCCcauaaucguauugaCUCCACGCcuauaua |
| | | cuucaaggACAACUUACCACGACCAUGGCUCAGACgUGUUAAAAUAUUACCACccuugaauauauagUUUGUUACcuguuuauug |
| | | acaugCCUAUUAACAGCGACCAUGGCUCAGACgUGUUAAAAUAUUCAUUCcaugucauaaacagAACUCUCC |
| x6 sites- | 495 | gggugauauacgaccaguUCACUCUUAAACGACCAUGGCUCAGACCUGUUAAAACCUUACAUUacuggucuauaucaAUAAUGUU |
| Imperf | | ugauaagaaaugacuUGAUAUCUACCCGACCAUGGCUCAGACCUGUUAAAAUGCUCCUAUagucauucuuaucaUUUACUCUca |
| C | | uuaguauguuaagAGACUAGAAUACGACCAUGGCUCAGACCUGUUAAAAUAUCCACAAcuuaacaacuaaugUAUGAACCucaau |
| | | acagauuaugAACUCACUCCACGACCAUGGCUCAGACCUGUUAAAACGCGCUACAcauaaucguauugaCUUAAUGAcuauauac |
| | | uucaaggCCACCUAACUGCGACCAUGGCUCAGACCUGUUAAAACUUCACCAAccuugaauauauagACUGAUCUcuguuuauuga |
| | | caugACAAGAAACUUCGACCAUGGCUCAGACCUGUUACUAAUAUACACCcaugucauaaacagUUCCUUGU |
| x6 sites- | 496 | gggugauauacgaccaguUGCACGCACUCCGACCAUGGCUCAGACuUGUUAAAAUUAUACACUacuggucuauaucaCCCUUACU |
| Imperf | | ugauaagaaaugacuCUAAAUGCACUCGACCAUGGCUCAGACuUGUUAAAUAUUCUACACagucauucuuaucaUUCGUCUCcau |
| U | | uaguauguuaagAUCUCUAUCUUCGACCAUGGCUCAGACuUGUUACACUUAAAUUCGcuuaacaacuaaugUCUCAACCucaaua |
| | | cagauuaugUGCAUUAAACCCGACCAUGGCUCAGACuUGUUAAAACUUCCAUCUcauaaucguauugaACAACUAUcuauauacu |
| | | ucaaggUACUAGAAAUCCGACCAUGGCUCAGACuUGUUAAAUAACCUCAAUccuugaauauauagUCUAAAUAcuguuuauugac |
| | | augCUCUAAGAACUCGACCAUGGCUCAGACuUGUUAAAUAACGCAUCCcaugucauaaacagGCUACAAC |
| x6 sites- | 497 | gggugauauacgaccaguCAACCAACUAUcgaccauggcucagacuguaaCAUUUAACUCAAacuggucuauaucaCCACUCCCugauaag |
| Imperf | | aaaugacuAUAUACUAAUUcgaccauggcucagacuguaaUAUAAGACAUCAagucauucuuaucaAAACUAUCcauuaguauguuaag |
| pos2 | | UGACACGCACCcgaccauggcucagacuguaaAUUCCAAAUCCUcuuaacaacuaaugCAUUCCAAucaauacagauuaugUAACGUAU |
| | | CACcgaccauggcucagacuguaaACAUUCCUCAUUcauaaucguauugaACAUCUACcuauauacuucaaggUUCCCUAAUGAcgacca |
| | | uggcucagacuguaaCAACGACCUACUccuugaauauauagAUACUAUCcuguuuauugacaugUCAAUUUCAACcgaccauggcucagac |
| | | uguaaUAUUCACCCGCUcaugucauaaacagACAACCUU |
| x6 sites- | 498 | gggugauauacgaccaguAAUACACUUCAcgaccauggcucagacugauaUUACUAUAAACCacuggucuauaucaCUACAAUAugauaag |
| Imperf | | aaaugacuUUUCCACACUAcgaccauggcucagacugauaUAAAUCUAACAUagucauucuuaucaAGCAACCAcauuaguauguuaagA |
| pos3 | | UACAUAAUUAcgaccauggcucagacugauaACCUUCCUUCCCcuuaacaacuaaugCUUUAAUAucaauacagauuaugUAAACUUA |
| | | ACUcgaccauggcucagacugauaUUUAAUACUAACcauaaucguauugaCCCUCCAAcuauauacuucaaggCUCCACACGAUcgaccau |
| | | ggcucagacugauaUAACAAUUCUUAccuugaauauauagCUUCAUACcuguuuauugacaugAAUCACAAGAAcgaccauggcucagac |
| | | ugauaAACCUAAUCCUCcaugucauaaacagUACCUUAU |
| x6 sites- | 499 | gggugauauacgaccaguUCAGACAAACAcgaccauggcucagacucuuaCUAUCUCAACACacuggucuauaucaCAUUUACAugauaag |
| Imperf | | aaaugacuUGAACAAACGAcgaccauggcucagacucuuaCUAAAUUCUCUUagucauucuuaucaCAUCUACUcauuaguauguuaagG |
| pos4 | | AACAGCAUUCcgaccauggcucagacucuuaGAUACAGUACCAcuuaacaacuaaugCAUCAAUAucaauacagauuaugGUAAACUAU |
| | | ACcgaccauggcucagacucuuaAUUACAUUCAAAcauaaucguauugaCCAUCUUGcuauauacuucaaggACACAACGUUAcgaccau |
| | | ggcucagacucuuaCAAACUUAUCACccuugaauauauagAUUUAACCcuguuuauugacaugGAUACCAAUUAcgaccauggcucagac |
| | | ucuuaCUUACACAACCGcaugucauaaacagCACAACCG |
| x6 sites- | 500 | gggugauauacgaccaguUAUACAGACUAcgaccauggcucagacaguuaCAAACGCGAACCacuggucuauaucaCACAAUUUugauaag |
| Imperf | | aaaugacuUUACAAAUAUCcgaccauggcucagacaguuaAAACCGCAACAUagucauucuuaucaUCCCGCAUcauuaguauguuaagC |
| pos5 | | UACAAAUCACcgaccauggcucagacaguuaGACACCUUCCCUcuuaacaacuaaugUUCUACACucaauacagauuaugUAAAUCCUA |
| | | CCcgaccauggcucagacaguuaACCUCUUUCACUcauaaucguauugaUAAUCAAUcuauauacuucaaggUACAAGCACGAcgaccaug |
| | | gcucagacaguuaAACCUCCACAAUccuugaauauauagCUUUAAUAcuguuuauugacaugAAGAAUCCCUAcgaccauggcucagacag |
| | | uuaCAUAACCUACAGcaugucauaaacagAACACAGA |
| x6 sites- | 501 | gggugauauacgaccaguUUUCUAUCCUUcgaccauggcucagaguguuaAUACUAUAUCAUacuggucuauaucaUCUAUCUUugaua |
| Imperf | | agaaaugacuAUCCUACCCGAcgaccauggcucagaguguua UAUCCAUUGACGagucauucuuaucaUUACCAACcauuaguauguuaa |
| pos6 | | gAAAUCUUCCACcgaccauggcucagaguguuaAACCAUCUACAGcuuaacaacuaaugUGCUCAUCucaauacagauuaugUGUUACU |
| | | UUCAcgaccauggcucagaguguuaUCUAUCAUAUCCcauaaucguauugaAGUCCUCUcuauauacuucaaggAGCAACCUUUAcgac |
| | | cauggcucagaguguuaAUCUUACGAAUCccuugaauauauagCGUCAACCcuguuuauugacaugUAUAAAUCACGcgaccauggcuca |
| | | gaguguuaCCAACAGAAUUUcaugucauaaacagAUCCUUCU |
| x6 sites- | 502 | gggugauauacgaccaguCUGCGAAUACCcgaccauggcucagucuguuaCCUUUGAUCUUAacuggucuauaucaUCUACCUCugauaa |
| Imperf | | gaaaugacuGUGAACAUUCCcgaccauggcucagucuguuaUCUAACGAAAUAagucauucuuaucaAACUCAACcauuaguauguuaag |
| pos7 | | AGAUACUUACUcgaccauggcucagucuguuaUCCUACCUUCCGcuuaacaacuaaugUACUCAUCucaauacagauuaugCUCCACUA |
| | | UUCcgaccauggcucagucuguuaAAACUCGAUCUUcauaaucguauugaUCUAGCCAcuauauacuucaaggACCAAAGCGCCcgacca |
| | | uggcucagucuguuaGUUAAUUCCAUAccuugaauauauagCCACUUAGcuguuuauugacaugGCCUCUCAUUGcgaccauggcucag |
| | | ucuguuaAUUCCUUCCAAGcaugucauaaacagAAUCUCCA |
| x6 sites- | 503 | gggugauauacgaccaguACCCUCAUCUAcgaccauggcucacacuguuaUUCUUAUCAUUAacuggucuauaucaUUCCAAAUugauaa |
| Imperf | | gaaaugacuCUGACUAUUCGcgaccauggcucacacuguuaAAUAUGAAACCUagucauucuuaucaCACUCAACcauuaguauguuaag |
| pos8 | | GCAAUCUACUUcgaccauggcucacacuguuaUACAACCUACGAcuuaacaacuaaugAAGCUCAGucaauacagauuaugCACGCUUC |
| | | AUUcgaccauggcucacacuguuaAUUAACUAAUCUcauaaucguauugaAUUCCACAcuauauacuucaaggACUAACACAUAcgacca |
| | | uggcucacacuguuaCAAGUCCUCUCCccuugaauauauagCCGCCUACcuguuuauugacaugAUUUACAUCUAcgaccauggcucacac |
| | | uguuaCACUCAAAUUACcaugucauaaacagUACCCUUC |
| x3 sites- | 504 | gggugauauacgaccaguUCCCCGACCAUGGCUCAGACUGUUAGAACacuggucuauaucaUGCGACUAugauaagaaaugacuACGA |
| 4/4 | | CGACCAUGGCUCAGACUGUUACUAAagucauucuuaucaAUCCGAAUcauuaguauguuaagCAUCCGACCAUGGCUCAGACUG |
| aux arms | | UUAAUUAcuuaacaacuaaugCUACCAUA |
| x3 sites- | 505 | gggugauauacgaccaguAUAACGCGACCAUGGCUCAGACUGUUACUACCCacuggucuauaucaUGCCCGAUugauaagaaaugac |
| 6/6 | | uGACACCCGACCAUGGCUCAGACUGUUAGCAAUAagucauucuuaucaUGAACUCAcauuaguauguuaagUUAUUCCGACCAU |
| aux arms | | GGCUCAGACUGUUAAUCACUcuuaacaacuaaugAACUAAAU |
| x3 sites- | 506 | gggugauauacgaccaguAAAUGCAACGACCAUGGCUCAGACUGUUAACUAAAUGacuggucuauaucaCACCUUCCugauaagaaa |
| 8/8 | | ugacuCCGCACGACGACCAUGGCUCAGACUGUUACAUACCCAagucauucuuaucaUACAUUCUcauuaguauguuaagUAUCAC |
| aux arms | | AACGACCAUGGCUCAGACUGUUAACCUACCUcuuaacaacuaaugUUACAACC |
| x3 sites- | 507 | gggugauauacgaccaguCUCCCUCCGAAUCGACCAUGGCUCAGACUGUUAACUAUAUUACACacuggucuauaucaAACUCCCA |
| 12/12 | | ugauaagaaaugacuUGCAACCUCUCACGACCAUGGCUCAGACUGUUAACUCCCUCAACUagucauucuuaucaGACCCUACcau |
| aux arms | | uaguauguuaagCGACAAUAAACACGACCAUGGCUCAGACUGUUAAUCUCACAACCUcuuaacaacuaaugCUUCUAUC |
| x6 sites- | 508 | gggugauauacgaccaguUCCCCGACCAUGGCUCAGACUGUUAGAACacuggucuauaucaUGCGACUAugauaagaaaugacuACG |
| 4/4 | | ACGACCAUGGCUCAGACUGUUACUAAagucauucuuaucaAUCCGAAUcauuaguauguuaagCAUCCGACCAUGGCUCAGACU |
| aux arms | | GUUAAUUAcuuaacaacuaaugCUACCAUAucaauacagauuaugUUCACGACCAUGGCUCAGACUGUUAAUACcauaaucguauu |
| | | gaCUCCCAUAcuauauacuucaaggUCCGCGACCAUGGCUCAGACUGUUACCUUccuugaauauauagAGAAAUUAcuguuuauug |
| | | acaugCACACGACCAUGGCUCAGACUGUUAAACCcaugucauaaacagACUCCCUG |
| x6 sites- | 509 | gggugauauacgaccaguAUAACGCGACCAUGGCUCAGACUGUUACUACCCacuggucuauaucaUGCCCGAUugauaagaaaugac |
| 6/6 | | uGACACCCGACCAUGGCUCAGACUGUUAGCAAUAagucauucuuaucaUGAACUCAcauuaguauguuaagUUAUUCCGACCAU |
| aux arms | | GGCUCAGACUGUUAAUCACUcuuaacaacuaaugAACUAAAUucaauacagauuaugAACUUACGACCAUGGCUCAGACUGUUA |
| | | CUUUCCcauaaucguauugaGAUCCAUAcuauauacuucaaggGUUCGACGACCAUGGCUCAGACUGUUACACAUAccuugaauau |
| | | auagACCACACCcuguuuauugacaugAAUAACCGACCAUGGCUCAGACUGUUAACCAAGcaugucauaaacagAACCACUG |
| x6 sites- | 510 | gggugauauacgaccaguAAAUGCAACGACCAUGGCUCAGACUGUUAACUAAAUGacuggucuauaucaCACCUUCCugauaagaaa |
| 8/8 | | ugacuCCGCACGACGACCAUGGCUCAGACUGUUACAUACCCAagucauucuuaucaUACAUUCUcauuaguauguuaagUAUCAC |
| aux arms | | AACGACCAUGGCUCAGACUGUUAACCUACCUcuuaacaacuaaugUUACAACCucaauacagauuaugACGAAAUACGACCAUGG |
| | | CUCAGACUGUUACACCUUAAcauaaucguauugaAAUCCCUCcuauauacuucaaggUACACUCCCGACCAUGGCUCAGACUGUU |
| | | ACAGACACUccuugaauauauagCGCACAACcuguuuauugacaugUUUAAGAACGACCAUGGCUCAGACUGUUACAACUCACca |
| | | ugucauaaacagCCCAUAGC |
| x6 sites- | 511 | gggugauauacgaccaguCCGAUCACUCUACGACCAUGGCUCAGACUGUUACACAUAUCCUAAacuggucuauaucaCAGCUAGC |
| 12/12 | | ugauaagaaaugacuAUCUACAACUUACGACCAUGGCUCAGACUGUUACCAAACACCUAAagucauucuuaucaUAUCUCUUcau |
| aux arms | | uaguauguuaagGCAUCACCGAAUCGACCAUGGCUCAGACUGUUAAACUCCUACAAAcuuaacaacuaaugCUCACUAUucaaua |
| | | cagauuaugACUAUCCUAAAUCGACCAUGGCUCAGACUGUUAACAAUUUCACCGcauaaucguauugaUACCAUACcuauauacu |
| | | ucaaggCACUCCUUCUCUCGACCAUGGCUCAGACUGUUACAUUAAUACCCAccuugaauauauagCAGAUCCCcuguuuauugac |
| | | augAUUACUAAUCUACGACCAUGGCUCAGACUGUUACUUAAUUUCCUAcaugucauaaacagACUCUCCA |

TABLE 10
miR-155 engineered sequences RNA

| Design | SEQ ID | |
|---|---|---|
| Description | NO: | Sequence |
| x1 site | 512 | gggUGAUAUACGACCAGUgcgcacuacacAACCCCUAUCACGUAAGCAUUAAgaaacucaagauACUGGUCUAUAUCAuuaugagc |
| x2 site | 513 | gggUGAUAUACGACCAGUgcgcacuacacAACCCCUAUCACGUAAGCAUUAAgaaacucaagauACUGGUCUAUAUCAuuaugagcU |
| | | GAUAAGAAAUGACUauccgaacuacAACCCCUAUCACGUAAGCAUUAAucauccaacaugAGUCAUUCUUAUCAuucagaca |
| x3 site | 514 | gggUGAUAUACGACCAGUgcgcacuacacAACCCCUAUCACGUAAGCAUUAAgaaacucaagauACUGGUCUAUAUCAuuaugagcU |
| | | GAUAAGAAAUGACUauccgaacuacAACCCCUAUCACGUAAGCAUUAAucauccaacaugAGUCAUUCUUAUCAuucagacaCAUU |
| | | AGUAUGUUAAGugacucaucauAACCCCUAUCACGUAAGCAUUAAcuacucaacgauCUUAACAACUAAUGuuuaguag |
| x4 site | 515 | gggUGAUAUACGACCAGUgcgcacuacacAACCCCUAUCACGUAAGCAUUAAgaaacucaagauACUGGUCUAUAUCAuuaugagcU |
| | | GAUAAGAAAUGACUauccgaacuacAACCCCUAUCACGUAAGCAUUAAucauccaacaugAGUCAUUCUUAUCAuucagacaCAUU |
| | | AGUAUGUUAAGugacucaucauAACCCCUAUCACGUAAGCAUUAAcuacucaacgauCUUAACAACUAAUGuuuaguagUCAAUAC |
| | | AGAUUAUGacagcaucuauAACCCCUAUCACGUAAGCAUUAAuacaccacgacgCAUAAUCGUAUUGAuucuuuca |
| x5 site | 516 | gggUGAUAUACGACCAGUgcgcacuacacAACCCCUAUCACGUAAGCAUUAAgaaacucaagauACUGGUCUAUAUCAuuaugagcU |
| | | GAUAAGAAAUGACUauccgaacuacAACCCCUAUCACGUAAGCAUUAAucauccaacaugAGUCAUUCUUAUCAuucagacaCAUU |
| | | AGUAUGUUAAGugacucaucauAACCCCUAUCACGUAAGCAUUAAcuacucaacgauCUUAACAACUAAUGuuuaguagUCAAUAC |
| | | AGAUUAUGacagcaucuauAACCCCUAUCACGUAAGCAUUAAuacaccacgacgCAUAAUCGUAUUGAuucuuucaCUAUAUACUU |
| | | CAAGGaccaucacuacAACCCCUAUCACGUAAGCAUUAAcacuacuagcagCCUUGAAUAUAUAGuucaugcc |
| x6 site | 517 | gggUGAUAUACGACCAGUgcgcacuacacAACCCCUAUCACGUAAGCAUUAAgaaacucaagauACUGGUCUAUAUCAuuaugagcU |
| | | GAUAAGAAAUGACUauccgaacuacAACCCCUAUCACGUAAGCAUUAAucauccaacaugAGUCAUUCUUAUCAuucagacaCAUU |
| | | AGUAUGUUAAGugacucaucauAACCCCUAUCACGUAAGCAUUAAcuacucaacgauCUUAACAACUAAUGuuuaguagUCAAUAC |
| | | AGAUUAUGacagcaucuauAACCCCUAUCACGUAAGCAUUAAuacaccacgacgCAUAAUCGUAUUGAuucuuucaCUAUAUACUU |
| | | CAAGGaccaucacuacAACCCCUAUCACGUAAGCAUUAAcacuacuagcagCCUUGAAUAUAUAGuucaugccCUGUUUAUUGACA |
| | | UGucacaucucacAACCCCUAUCACGUAAGCAUUAAcacaucacgcauCAUGUCAUAAACAGuuauacaa |
| x3 sites- | 518 | gggugauauacgaccaguAACAUAUAACCaaccccuaucacguaagcauuaaACUAACUAUUCCacuggucuauaucaCCugauaagaaaugac |
| 2 nt | | uUACAUUCUCAAaaccccuaucacguaagcauuaaGAUACACCGAACagucauucuuaucaCAcauuaguauguuaagAUAUAGCAACAaa |
| linker | | ccccuaucacguaagcauuaaCCUCACAGACCAcuuaacaacuaaugCC |
| x3 sites- | 519 | gggugauauacgaccaguUAAAGCAAUCUaaccccuaucacguaagcauuaaGCACCUCUCACUacuggucuauaucaCCUAugauaagaaau |
| 4 nt | | gacuUUCCACUCGCAaaccccuaucacguaagcauuaaUACCUACCUCAUagucauucuuaucaAUAAcauuaguauguuaagUAAUCCAC |
| linker | | UCUaaccccuaucacguaagcauuaaACCACUCUGACCcuuaacaacuaaugCCAA |
| x3 sites- | 520 | gggugauauacgaccaguCAACCUAAUUCaaccccuaucacguaagcauuaaCCAUCUCCUAAAacuggucuauaucaUAUACAugauaagaa |
| 6 nt | | augacuCAUUCUUAGCCaaccccuaucacguaagcauuaaUCGAUCCGCCAAagucauucuuaucaCAACCGcauuaguauguuaagACUCU |
| linker | | ACUCUAaaccccuaucacguaagcauuaaUUAUACUCCACCcuuaacaacuaaugAUCUAU |
| x3 sites- | 521 | gggugauauacgaccaguCCCGACCAGUUaaccccuaucacguaagcauuaaAUGAACAGAAAUacuggucuauaucaUCAAAUCCugauaa |
| 8 nt | | gaaaugacuUUACCACAUUCaaccccuaucacguaagcauuaaUUACCUUCCAUCaguauucuuaucaCCAUCACCcauuaguauguuaag |
| linker | | UACAUUCCAAGaaccccuaucacguaagcauuaaAUCACGACUCGCcuuaacaacuaaugCAAUUCAA |
| x6 sites- | 522 | gggugauauacgaccaguAACAUAUAACCaaccccuaucacguaagcauuaaACUAACUAUUCCacuggucuauaucaCCugauaagaaaugac |
| 2 nt | | uUACAUUCUCAAaaccccuaucacguaagcauuaaGAUACACCGAACagucauucuuaucaCAcauuaguauguuaagAUAUAGCAACAaa |
| linker | | ccccuaucacguaagcauuaaCCUCACAGACCAcuuaacaacuaaugCCucaauacagauuaugAAACUAUAUCCaaccccuaucacguaagcau |
| | | uaaUGUCGCCACACCcauaaucguauugaACcuauauacuucaaggAUACAAUCAAAaaccccuaucacguaagcauuaaACAAAUACAUU |
| | | GccuugaauauauagCUcuguuuauugacaugCCCAUUAUAUCaaccccuaucacguaagcauuaaAGUCUCUGACCUcaugucauaaacagC |
| | | G |
| x6 sites- | 523 | gggugauauacgaccaguUAAAGCAAUCUaaccccuaucacguaagcauuaaGCACCUCUCACUacuggucuauaucaCCUAugauaagaaau |
| 4 nt | | gacuUUCCACUCGCAaaccccuaucacguaagcauuaaUACCUACCUCAUagucauucuuaucaAUAAcauuaguauguuaagUAAUCCAC |
| linker | | UCUaaccccuaucacguaagcauuaaACCACUCUGACCcuuaacaacuaaugCCAAucaauacagauuaugCACUUCAAUUUaaccccuauca |
| | | cguaagcauuaaCCAACCUUUCUUcauaaucguauugaCCAUcuauauacuucaaggCUACCCUAAUCaaccccuaucacguaagcauuaaCC |
| | | UUCACCCUCAccuugaauauauagCGCCcuguuuauugacaugUCCGUAAACCGaaccccuaucacguaagcauuaaGACCCAAUCAAUca |
| | | ugucauaaacagUCUA |
| x6 sites- | 524 | gggugauauacgaccaguCAACCUAAUUCaaccccuaucacguaagcauuaaCCAUCUCCUAAAacuggucuauaucaUAUACAugauaagaa |
| 6 nt | | augacuCAUUCUUAGCCaaccccuaucacguaagcauuaaUCGAUCCGCCAAagucauucuuaucaCAACCGcauuaguauguuaagACUCU |
| linker | | ACUCUAaaccccuaucacguaagcauuaaUUAUACUCCACCcuuaacaacuaaugAUCUAUucaauacagauuaugAACCUCAAUUAaaccc |
| | | cuaucacguaagcauuaaCCUUUCAAUAUCcauaaucguauugaUUUCAAcuauauacuucaaggCCCACACCGUCaaccccuaucacguaag |
| | | cauuaaCCGUUCCAUCACccuugaauauauagACCAAAcuguuuauugacaugAAACCUUAAUAaaccccuaucacguaagcauuaaCAUAU |
| | | UCCCUAGcaugucauaaacagCUCUAU |
| x6 sites- | 525 | gggugauauacgaccaguACAUUCCGCAAaaccccuaucacguaagcauuaaCAACUGACCGAAacuggucuauaucaUAUAACCUugauaag |
| 8 nt | | aaaugacuUCACUUCAGCCaaccccuaucacguaagcauuaaGAUAUAACCAAUagucauucuuaucaCAAUUCUCcauuaguauguuaagC |
| linker | | CACCGUCGCAaaccccuaucacguaagcauuaaACCUAGUUUCAUcuuaacaacuaaugACUUUCUCucaauacagauuaugUAACUUUA |
| | | ACCaaccccuaucacguaagcauuaaUUAGCCACAAAUcauaaucguauugaUAUUCCUUcuauauacuucaaggUAAUUACCGUCaacccc |
| | | uaucacguaagcauuaaCCAUAAUCGACUccuugaauauauagUAUAGACUcuguuuauugacaugCCUCCAAUUCUaaccccuaucacgua |
| | | agcauuaaAUACAACUUCAAcaugucauaaacagCCCAGCAC |
| x3 sites- | 526 | ggguauacgaccaguUUUCAAUACUUaaccccuaucacguaagcauuaaCUACCGCAACUCacuggucguauaUUUACCUCugauaagaaau |
| 12 nt | | gACCGUACCCUCaaccccuaucacguaagcauuaaACCGACUAAUACcauuucuuaucaCCCUUAUAcauuaguauguuCACGCCAAUAA |
| ND | | aaccccuaucacguaagcauuaaAGUCCUACCAAUaacauacuaaugAUUUAUUC |
| x3 sites- | 527 | ggguauacgaccaguGCCCUAUACUUaaccccuaucacguaagcauuaaCGACCUCUAAGAacugguguauaUCCCUCUAugauaagaaaug |
| 12 nt | | ACAGUAGCAUUaaccccuaucacguaagcauuaaAUUCCCUCUAGCcauuucuaucaAUUAUCAGcauuaguauguuUCUUCCGCUCCa |
| BD | | accccuaucacguaagcauuaaUUUAUCCGCCAUaacauauaaugACAAUCCA |
| x3 sites- | 528 | ggguauacgaccaguCCAUAUCCAUCaaccccuaucacguaagcauuaaUAUCCUAAGAUCacuggugguauaCGUUCCACugauaagaaau |
| 12 nt | | gCCUCAAGAACAaaccccuaucacguaagcauuaaUGACUUCCAAUAcauuucauaucaACCAUCACcauuaguauguuUAUUCCAUCUC |
| MM | | aaccccuaucacguaagcauuaaUCCAUUUCGACUaacauaguaaugUCACCAUA |
| x3 sites- | 529 | gggugauauacgaccaguCUAUUUCCUAGaaccccuaucacguaagcauuaaAGACUCACAACCacuggucguauaucaCCGACUUCugauaa |
| 15 nt | | gaaaugacuCAAAUACUAAUaaccccuaucacguaagcauuaaAUUCAAGAACUCagucauuucuuaucaAAUAUUCCcauuaguauguuaa |
| ND | | gAUCAUUUACCGaaccccuaucacguaagcauuaaACAGACCACAAAcuuaacauacuaaugUGUUACGU |
| x3 sites- | 530 | gggugauauacgaccaguUUAACAAAUAAaaccccuaucacguaagcauuaaGUAACUAUCUCUacuggucuauaucaAAGAAAUCugauaa |
| 15 nt | | gaaaugacuACCCUUCAGCAaaccccuaucacguaagcauuaaAUUCCCUCUUCAagucauucuuaucaCAACAUGUcauuaguauguuaag |
| BD | | UCAAUCUAGCAaaccccuaucacguaagcauuaaACAACUUGUCCUcuuaacaacuaaugUCCUCACA |
| x3 sites- | 531 | gggugauauacgaccaguUUAAUUCCAACaaccccuaucacguaagcauuaaAGUAAAUCGCACacugguccuauaucaACCCAACCugauaa |
| 15 nt | | gaaaugacuUAGCCUAACAUaaccccuaucacguaagcauuaaGUCCGUAACCACagucauuacuuaucaUGAGUCCUcauuaguauguuaa |
| MM | | gUCUCAGCCAGAaaccccuaucacguaagcauuaaAUCAAUCUAUAUcuuaacaaacuaaugUCACCGUU |
| x6 sites- | 532 | ggguauacgaccaguUUAACCAUCCAaaccccuaucacguaagcauuaaGAGCCACUAUUUacuggucguauaCCAUUGCAugauaagaaau |
| 12 nt | | gCCCGAUCUGUCaaccccuaucacguaagcauuaaGUACCAACCCUAcauuucuuaucaCAUACACCcauuaguauguuCCUCCACGCCUa |
| ND | | accccuaucacguaagcauuaaCCUUCGAAAUCUaacauacuaaugUUCAUACCucaauacagauuAGACCCAACCUaaccccuaucacguaa |
| | | gcauuaaACAACUUCACCCaaucuguauugaUCCCACACcuauauacuucaGAAUACAACCGaaccccuaucacguaagcauuaaCCUCUAG |
| | | CCCAGugaaguauauagAUCACAAGcuguuuauugacCCAUAUUCAAGaaccccuaucacguaagcauuaaCUCAACGAUUUAgucaauaa |
| | | acagACACCAGA |
| x6 sites- | 533 | ggguauacgaccaguGCCCUAUACUUaaccccuaucacguaagcauuaaCGACCUCUAAGAacugguguauaUCCCUCUAugauaagaaaug |
| 12 nt | | ACAGUAGCAUUaaccccuaucacguaagcauuaaAUUCCCUCUAGCcauuucuaucaAUUAUCAGcauuaguauguuUCUUCCGCUCCa |
| BD | | accccuaucacguaagcauuaaUUUAUCCGCCAUaacauauaaugACAAUCCAucaauacagauuCAAUUCAAACUaaccccuaucacguaag |
| | | cauuaaCCACUUCCCGUAaaucugauugaACCUUCAGcuauauacuucaUAAUAUUCCGCaaccccuaucacguaagcauuaaAAGAACAA |
| | | ACUUugaaguuauagAUACCUCAcuguuuauugacCCCAAGCACUUaaccccuaucacguaagcauuaaAACGACCAGUCCgucaauaacag |
| | | ACAAUUUA |
| x6 sites- | 534 | ggguauacgaccaguCCAUAUCCAUCaaccccuaucacguaagcauuaaUAUCCUAAGAUCacuggugguauaCGUUCCACugauaagaaau |
| 12 nt | | gCCUCAAGAACAaaccccuaucacguaagcauuaaUGACUUCCAAUAcauuucauaucaACCAUCACcauuaguauguuUAUUCCAUCUC |
| MM | | aaccccuaucacguaagcauuaa UCCAUUUCGACUaacauaguaaugUCACCAUAucaauacagauuCAUAUAAAUAAaaccccuaucacgu |
| | | aagcauuaaAUUCCCAACUCUaaucugaauugaAAUUACUUcuauauacuucaUUCAGCCCAGCaaccccuaucacguaagcauuaaCCCA |
| | | CAGUCACCugaaguuuauagUCCAGAAAcuguuuauugacAUCAGUCCCAGaaccccuaucacguaagcauuaaAUCCAAUAGUAAguca |
| | | auuaacagGCCAAUAU |
| x6 sites- | 535 | gggugauauacgaccaguCUAUUUCCUAGaaccccuaucacguaagcauuaaAGACUCACAACCacuggucguauaucaCCGACUUCugauaa |
| 15 nt | | gaaaugacuCAAAUACUAAUaaccccuaucacguaagcauuaaAUUCAAGAACUCagucauuucuuaucaAAUAUUCCcauuaguauguuaa |
| ND | | gAUCAUUUACCGaaccccuaucacguaagcauuaaACAGACCACAAAcuuaacauacuaaugUGUUACGUucaauacagauuaugUUCCAC |
| | | GCAGAaaccccuaucacguaagcauuaaCAAAGACCGACUcauaaucuguauugaCCAAUACUcuauauacuucaaggAACAAACACCGaac |
| | | cccuaucacguaagcauuaaUUUCACAUAGAAccuugaaguauauagUGCUCACUcuguuuauugacaugCCGUACCGCCCaaccccuauca |
| | | cguaagcauuaaAUAACAGUCAACcaugucaauaaacagCAAAUUAC |
| x6 sites- | 536 | gggugauauacgaccaguUUAACAAAUAAaaccccuaucacguaagcauuaaGUAACUAUCUCUacuggucuauaucaAAGAAAUCugauaa |
| 15 nt | | gaaaugacuACCCUUCAGCAaaccccuaucacguaagcauuaaAUUCCCUCUUCAagucauucuuaucaCAACAUGUcauuaguauguuaag |
| BD | | UCAAUCUAGCAaaccccuaucacguaagcauuaaACAACUUGUCCUcuuaacaacuaaugUCCUCACAucaauacagauuaugUCGCCUG |
| | | UCCUaaccccuaucacguaagcauuaaAUCCCUCUAAUUcauaaucguauugaCUAACUCGcuauauacuucaaggACCGACUCACAaaccc |
| | | cuaucacguaagcauuaaGAAAUCACUGAAccuugaauauauagAUAUAAACcuguuuauugacaugCCACUUCAUUAaaccccuaucacgu |
| | | aagcauuaaCCAUUCGUCUCAcaugucauaaacagUAUCCAGU |
| x6 sites- | 537 | gggugauauacgaccaguUUAAUUCCAACaaccccuaucacguaagcauuaaAGUAAAUCGCACacugguccuauaucaACCCAACCugauaa |
| 15 nt | | gaaaugacuUAGCCUAACAUaaccccuaucacguaagcauuaaGUCCGUAACCACagucauuacuuaucaUGAGUCCUcauuaguauguuaa |
| MM | | gUCUCAGCCAGAaaccccuaucacguaagcauuaaAUCAAUCUAUAUcuuaacaaacuaaugUCACCGUUucaauacagauuaugAACAUC |
| | | UCAAGaaccccuaucacguaagcauuaaACCAAACAUCGAcauaaucaguauugaUUUCAUUAcuauauacuucaaggCUCCAUACCAAaa |
| | | ccccuaucacguaagcauuaaAACUCGAAGACAccuugaacuauauagACCGCGUAcuguuuauugacaugAAACCGAACUAaaccccuauca |
| | | cguaagcauuaaUAUCAUGACCGAcaugucauuaaacagCCUUCCUG |
| x3 sites- | 538 | gggUGAUAUACGACCAGUgcgcacuacacAACCCCUAUCACGAUUAGCAUUAAgaaacucaagauACUGGUCUAUAUCAuuaugagc |
| Perf | | UGAUAAGAAAUGACUacuuuaacuacAACCCCUAUCACGAUUAGCAUUAAgaacacGacauCAGUCAUUCUUAUCAuucagacaCA |
| ctrl | | UUAGUAUGUUAAGuuaccgaucauAACCCCUAUCACGAUUAGCAUUAAcuuacaaacgauCUUAACAACUAAUGuuuaguag |
| x3 sites- | 539 | gggUGAUAUACGACCAGUgcgcacuacacAACCCCUAUCACGAUUAGCaAUUAAgaaacucaagauACUGGUCUAUAUCAuuaugag |
| Perf A | | CUGAUAAGAAAUGACUacuuuaacuacAACCCCUAUCACGAUUAGCaAUUAAgaacacGacauCAGUCAUUCUUAUCAuucagaca |
| | | CAUUAGUAUGUUAAGuuaagcaucauAACCCCUAUCACGAUUAGCaAUUAAcaacgaaacgauCUUAACAACUAAUGuuuaguag |
| x3 sites- | 540 | gggUGAUAUACGACCAGUucauacuacacAACCCCUAUCACGAUUAGCgAUUAAgaaacucaauauACUGGUCUAUAUCAuuaugag |
| Perf G | | cUGAUAAGAAAUGACUacaccgacauaAACCCCUAUCACGAUUAGCgAUUAAccuacagagauuAGUCAUUCUUAUCAuucagacaC |
| | | AUUAGUAUGUUAAGugacucaucauAACCCCUAUCACGAUUAGCgAUUAAauucagaacgauCUUAACAACUAAUGuuuaguag |
| x3 sites- | 541 | gggUGAUAUACGACCAGUgcgcacuacacAACCCCUAUCACGAUUAGCcAUUAAgaaacucaagauACUGGUCUAUAUCAuuaugag |
| Perf C | | CUGAUAAGAAAUGACUacuuuaacuacAACCCCUAUCACGAUUAGCcAUUAAgaacacGacauCAGUCAUUCUUAUCAuucagaca |
| | | CAUUAGUAUGUUAAGuuaagcaucauAACCCCUAUCACGAUUAGCcAUUAAcaacgaaacgauCUUAACAACUAAUGuuuaguag |
| x3 sites- | 542 | gggUGAUAUACGACCAGUgcgcacuacacAACCCCUAUCACGAUUAGCuAUUAAgaaacucaagauACUGGUCUAUAUCAuuaugag |
| Perf U | | CUGAUAAGAAAUGACUacuuuaacuacAACCCCUAUCACGAUUAGCuAUUAAgaacacGacauCAGUCAUUCUUAUCAuucagaca |
| | | CAUUAGUAUGUUAAGuuaccgaucauAACCCCUAUCACGAUUAGCuAUUAAcuuacaaacgauCUUAACAACUAAUGuuuaguag |
| x3 sites- | 543 | gggugauauacgaccaguAUUCUUAACCCaaccccuaucacgauuagcauuuaCACUUCGCAUCAacuggucuauaucauuaugagcugauaag |
| Perf | | aaaugacuACCAUCACCUCaaccccuaucacgauuagcauuuaUAACUAUAUUUAagucauucuuaucauucagacacauuaguauguuaagU |
| pos2 | | UCUCACGACCaaccccuaucacgauuagcauuuaAAUAGCACAUAUcuuaacaacuaauguuuaguag |
| x3 sites- | 544 | gggugauauacgaccaguGUCACAUAAAUaaccccuaucacgauuagcauaaaACCGAGAAUAAAacuggucuauaucauuaugagcugauaag |
| Perf | | aaaugacuCCAUCACUUCAaaccccuaucacgauuagcauaaaAAUUCUCAAUCUagucauucuuaucauucagacacauuaguauguuaagC |
| pos3 | | AUUCAACCCUaaccccuaucacgauuagcauaaaUUACGCCUUUCCcuuaacaacuaauguuuaguag |
| x3 sites- | 545 | gggugauauacgaccaguUUUCAGCCUCAaaccccuaucacgauuagcaauaaCUCCUUCCCACCacuggucuauaucauuaugagcugauaag |
| Perf | | aaaugacuCCAACCUCUAAaaccccuaucacgauuagcaauaaCACUCAAACUCUagucauucuuaucauucagacacauuaguauguuaagAG |
| pos4 | | AAACGCCUUaaccccuaucacgauuagcaauaaAUACUGAGCCAAcuuaacaacuaauguuuaguag |
| x3 sites- | 546 | gggugauauacgaccaguCUAACUUACUAaaccccuaucacgauuagcuuuaaAUAUCACAUUCCacuggucuauaucauuaugagcugauaag |
| Perf | | aaaugacuCGAAUUUCCGCaaccccuaucacgauuagcuuuaaCCUAUACCACACagucauucuuaucauucagacacauuaguauguuaagU |
| pos5 | | AAACACUAACaaccccuaucacgauuagcuuuaaCUCACUUUCGACcuuaacaacuaauguuuaguag |
| x3 sites- | 547 | gggugauauacgaccaguUUAACAUUUCAaaccccuaucacgauuaggauuaaCAUCACAACCUCacuggucuauaucauuaugagcugauaag |
| Perf | | aaaugacuUAAACUCACUCaaccccuaucacgauuaggauuaaUACCGCUACCCUagucauucuuaucauucagacacauuaguauguuaagCC |
| pos6 | | CAUACAUCAaaccccuaucacgauuaggauuaaAUACACGCCAACcuuaacaacuaauguuuaguag |
| x3 sites- | 548 | gggugauauacgaccaguAUUCUACAACCaaccccuaucacgauuaccauuaaCUAUAGCCUCACacuggucuauaucauuaugagcugauaag |
| Perf | | aaaugacuCAUAAACACAUaaccccuaucacgauuaccauuaaCCAGCAUAACACagucauucuuaucauucagacacauuaguauguuaagCG |
| pos7 | | CUCUACUUUaaccccuaucacgauuaccauuaaCUAACGAAACACcuuaacaacuaauguuuaguag |
| x3 sites- | 549 | gggugauauacgaccaguAGACUCCACGAaaccccuaucacgauuugcauuaaCAUACACUUAACacuggucuauaucauuaugagcugauaag |
| Perf | | aaaugacuGAACUACAUAUaaccccuaucacgauuugcauuaaAUACACCCACUAagucauucuuaucauucagacacauuaguauguuaagU |
| pos8 | | AACCUUACGAaaccccuaucacgauuugcauuaaUAAACAAUAUACcuuaacaacuaauguuuaguag |
| x3 sites- | 514 | gggUGAUAUACGACCAGUgcgcacuacacAACCCCUAUCACGUAAGCAUUAAgaaacucaagauACUGGUCUAUAUCAuuaugagcU |
| Imperf | | GAUAAGAAAUGACUauccgaacuacAACCCCUAUCACGUAAGCAUUAAucauccaacaugAGUCAUUCUUAUCAuucagacaCAUU |
| ctrl | | AGUAUGUUAAGugacucaucauAACCCCUAUCACGUAAGCAUUAAcuacucaacgauCUUAACAACUAAUGuuuaguag |
| x3 sites- | 550 | gggUGAUAUACGACCAGUgcgcacuacacAACCCCUAUCACGUAAGCaAUUAAgaaacucaagauACUGGUCUAUAUCAuuaugagc |
| Imperf | | UGAUAAGAAAUGACUauccgaacuacAACCCCUAUCACGUAAGCaAUUAAucauccaacaugAGUCAUUCUUAUCAuucagacaCAU |
| A | | UAGUAUGUUAAGugacucaucauAACCCCUAUCACGUAAGCaAUUAAcuacucaacgauCUUAACAACUAAUGuuuaguag |
| x3 sites- | 551 | gggUGAUAUACGACCAGUucagacuacacAACCCCUAUCACGUAAGCgAUUAAgaaacucaauauACUGGUCUAUAUCAuuaugagc |
| Imperf | | UGAUAAGAAAUGACUacaccgacauaAACCCCUAUCACGUAAGCgAUUAAccuacagagauuAGUCAUUCUUAUCAuucagacaCAU |
| G | | UAGUAUGUUAAGugacucagcauAACCCCUAUCACGUAAGCgAUUAAauucagaacaauCUUAACAACUAAUGuuuaguag |
| x3 sites- | 552 | gggUGAUAUACGACCAGUgcgcacuacacAACCCCUAUCACGUAAGCcAUUAAgaaacucaagauACUGGUCUAUAUCAuuaugagc |
| Imperf | | UGAUAAGAAAUGACUauccgaacuacAACCCCUAUCACGUAAGCcAUUAAucauccaacaugAGUCAUUCUUAUCAuucagacaCAU |
| C | | UAGUAUGUUAAGugacucaucauAACCCCUAUCACGUAAGCcAUUAAcuacucaacgauCUUAACAACUAAUGuuuaguag |
| x3 sites- | 553 | gggUGAUAUACGACCAGUgcgcacuacacAACCCCUAUCACGUAAGCuAUUAAgaaacucaagauACUGGUCUAUAUCAuuaugagc |
| Imperf | | UGAUAAGAAAUGACUauccgaacuacAACCCCUAUCACGUAAGCuAUUAAucauccaacaugAGUCAUUCUUAUCAuucagacaCA |
| U | | UUAGUAUGUUAAGugacucaucauAACCCCUAUCACGUAAGCuAUUAAcuacucaacgauCUUAACAACUAAUGuuuaguag |
| x3 sites- | 554 | gggugauauacgaccaguACCAUCUCCGCaaccccuaucacguaagcauuuaAAGUUCCCAGCCacuggucuauaucaAACACCAAugauaag |
| Imperf | | aaaugacuUUACAUUAACGaaccccuaucacguaagcauuuaAUAUAAUUACUUagucauucuuaucaGAAACUACcauuaguauguuaag |
| pos2 | | CACUUAAACCAaaccccuaucacguaagcauuuaCCAAAUCGACUAcuuaacaacuaaugCCAACUUA |
| x3 sites- | 555 | gggugauauacgaccaguCCUCAAUCGCAaaccccuaucacguaagcauaaaCCUAAACAUACUacuggucuauaucaCAAUUAACugauaag |
| Imperf | | aaaugacuCGCCAUAACAGaaccccuaucacguaagcauaaaGAAACUUCCAUUagucauucuuaucaACCUGAACcauuaguauguuaagC |
| pos3 | | AACUCCAAUAaaccccuaucacguaagcauaaaCAAGACCGACCAcuuaacaacuaaugAGCCGCAU |
| x3 sites- | 556 | gggugauauacgaccaguAAACCAACCUAaaccccuaucacguaagcaauaaGCCAUUUCCACCacuggucuauaucaGAAACCACugauaag |
| Imperf | | aaaugacuAUCCAUCAGUCaaccccuaucacguaagcaauaaCACCAUUAAGCCagucauucuuaucaAUCUUCACcauuaguauguuaagA |
| pos4 | | UUCCGCAGUCaaccccuaucacguaagcaauaaCAUCUUAACUUCcuuaacaacuaaugCUACUUCC |
| x3 sites- | 557 | gggugauauacgaccaguCGACAUCUACUaaccccuaucacguaagcuuuaaUAAUCUACCUAAacuggucuauaucaAUCAUUCCugauaa |
| Imperf | | gaaaugacuCCUCCCAUUAAaaccccuaucacguaagcuuuaaAUUAGACAUAACagucauucuuaucaAUACCUACcauuaguauguuaag |
| pos5 | | CAUCCUAUACUaaccccuaucacguaagcuuuaaCCUUCUGUACCUcuuaacaacuaaugAACUACCC |
| x3 sites- | 558 | gggugauauacgaccaguCUCACUUAAACaaccccuaucacguaaggauuaaAGACACCGCACAacuggucuauaucaUGCUAAACugauaag |
| Imperf | | aaaugacuGCCUUUAACACaaccccuaucacguaaggauuaaUAACGCCCAAUGagucauucuuaucaCCUUUGUUcauuaguauguuaag |
| pos6 | | ACCCAAGAUCAaaccccuaucacguaaggauuaaAUAACGACAAACcuuaacaacuaaugCCACCUGU |
| x3 sites- | 559 | gggugauauacgaccaguCCAACACCCGAaaccccuaucacguaaccauuaaGCAUUCCGAUUCacuggucuauaucaAUCUCACCugauaag |
| Imperf | | aaaugacuAAAUCCAUUAUaaccccuaucacguaaccauuaaCUCCUGUCUAGAagucauucuuaucaAACUAAUCcauuaguauguuaag |
| pos7 | | AAAUCACUAUAaaccccuaucacguaaccauuaaUUAAUCAAUCACcuuaacaacuaaugAUUCAACC |
| x3 sites- | 560 | gggugauauacgaccaguACUCCAAUCAAaaccccuaucacguaugcauuaaCUACUUACUUCAacuggucuauaucaUCUCUACCugauaag |
| Imperf | | aaaugacuACACGCAAUCAaaccccuaucacguaugcauuaaAUCACAACCCUCagucauucuuaucaCACAUUCCcauuaguauguuaagA |
| pos8 | | AUUUCUCUACaaccccuaucacguaugcauuaaUCAAACUAAUCGcuuaacaacuaaugAAUCAAUU |
| x6 sites- | 561 | gggUGAUAUACGACCAGUgcgcacuacacAACCCCUAUCACGAUUAGCAUUAAgaaacucaagauACUGGUCUAUAUCAuuaugagc |
| Perf | | UGAUAAGAAAUGACUacuuuaacuacAACCCCUAUCACGAUUAGCAUUAAgaacacGacauCAGUCAUUCUUAUCAuucagacaCA |
| ctrl | | UUAGUAUGUUAAGuuaccgaucauAACCCCUAUCACGAUUAGCAUUAAcuuacaaacgauCUUAACAACUAAUGuuuaguagUCAA |
| | | UACAGAUUAUGacaucacgcauAACCCCUAUCACGAUUAGCAUUAAuacaccacgacgCAUAAUCGUAUUGAuucuuucaCUAUAU |
| | | ACUUCAAGGacucuaucuacAACCCCUAUCACGAUUAGCAUUAAcacuucacgccuCCUUGAAUAUAUAGuucaugccCUGUUUAU |
| | | UGACAUGucacaucucacAACCCCUAUCACGAUUAGCAUUAAcacaucacgcauCAUGUCAUAAACAGuuauacaa |
| x6 sites- | 584 | gggUGAUAUACGACCAGUgcgcacuacacAACCCCUAUCACGAUUAGCaAUUAAgaaacucaagauACUGGUCUAUAUCAuuaugag |
| Perf A | | cUGAUAAGAAAUGACUacuuuaacuacAACCCCUAUCACGAUUAGCaAUUAAgaacacGacauCAGUCAUUCUUAUCAuucagaca |
| | | CAUUAGUAUGUUAAGuuaagcaucauAACCCCUAUCACGAUUAGCaAUUAAcaacgaaacgauCUUAACAACUAAUGuuuaguagU |
| | | CAAUACAGAUUAUGacagcaucuauAACCCCUAUCACGAUUAGCaAUUAAuacaccacgacgCAUAAUCGUAUUGAuucuuucaCU |
| | | AUAUACUUCAAGGaccaucacuacAACCCCUAUCACGAUUAGCaAUUAAcacuacuagcagCCUUGAAUAUAUAGuucaugccCUGU |
| | | UUAUUGACAUGucacaucucacAACCCCUAUCACGAUUAGCaAUUAAcacaucacgcauCAUGUCAUAAACAGuuauacaa |
| x6 sites- | 303 | gggUGAUAUACGACCAGUucauacuacacAACCCCUAUCACGAUUAGCgAUUAAgaaacucaauauACUGGUCUAUAUCAuuaugag |
| Perf G | | CUGAUAAGAAAUGACUacaccgacauaAACCCCUAUCACGAUUAGCgAUUAAccuacagagauuAGUCAUUCUUAUCAuucagacaC |
| | | AUUAGUAUGUUAAGugacucaucauAACCCCUAUCACGAUUAGCgAUUAAauucagaacgauCUUAACAACUAAUGuuuaguagUC |
| | | AAUACAGAUUAUGacagcaacuauAACCCCUAUCACGAUUAGCgAUUAAuacaccacgaauCAUAAUCGUAUUGAuucuuucaCUA |
| | | UAUACUUCAAGGacauaccuaucAACCCCUAUCACGAUUAGCgAUUAAcaccaugaccagCCUUGAAUAUAUAGuucaugccCUGU |
| | | UUAUUGACAUGucacaucucacAACCCCUAUCACGAUUAGCgAUUAAcacuccacucauCAUGUCAUAAACAGuuauacaa |
| x6 sites- | 304 | gggUGAUAUACGACCAGUgcgcacuacacAACCCCUAUCACGAUUAGCcAUUAAgaaacucaagauACUGGUCUAUAUCAuuaugag |
| Perf C | | CUGAUAAGAAAUGACUacuuuaacuacAACCCCUAUCACGAUUAGCcAUUAAgaacacGacauCAGUCAUUCUUAUCAuucagaca |
| | | CAUUAGUAUGUUAAGuuaagcaucauAACCCCUAUCACGAUUAGCcAUUAAcaacgaaacgauCUUAACAACUAAUGuuuaguagU |
| | | CAAUACAGAUUAUGacagcaucuauAACCCCUAUCACGAUUAGCcAUUAAuacaccacgacgCAUAAUCGUAUUGAuucuuucaCU |
| | | AUAUACUUCAAGGaccaucacuacAACCCCUAUCACGAUUAGCcAUUAAcacuacuagcagCCUUGAAUAUAUAGuucaugccCUGU |
| | | UUAUUGACAUGucacaucucacAACCCCUAUCACGAUUAGCcAUUAAcacaucacgcauCAUGUCAUAAACAGuuauacaa |
| x6 sites- | 305 | gggUGAUAUACGACCAGUgcgcacuacacAACCCCUAUCACGAUUAGCuAUUAAgaaacucaagauACUGGUCUAUAUCAuuaugag |
| Perf U | | CUGAUAAGAAAUGACUacuuuaacuacAACCCCUAUCACGAUUAGCuAUUAAgaacacGacauCAGUCAUUCUUAUCAuucagaca |
| | | CAUUAGUAUGUUAAGuuaccgaucauAACCCCUAUCACGAUUAGCuAUUAAcuuacaaacgauCUUAACAACUAAUGuuuaguagU |
| | | CAAUACAGAUUAUGacaucacgcauAACCCCUAUCACGAUUAGCuAUUAAuacaccacgacgCAUAAUCGUAUUGAuucuuucaCU |
| | | AUAUACUUCAAGGacucuaucuacAACCCCUAUCACGAUUAGCuAUUAAcacuucacgccuCCUUGAAUAUAUAGuucaugccCUG |
| | | UUUAUUGACAUGucacaucucacAACCCCUAUCACGAUUAGCuAUUAAcacaucacgcauCAUGUCAUAAACAGuuauacaa |
| x6 sites- | 562 | gggugauauacgaccaguAUUCUUAACCCaaccccuaucacgauuagcauuuaCACUUCGCAUCAacuggucuauaucauuaugagcugauaag |
| Perf | | aaaugacuACCAUCACCUCaaccccuaucacgauuagcauuuaUAACUAUAUUUAagucauucuuaucauucagacacauuaguauguuaagU |
| pos2 | | UCUCACGACCaaccccuaucacgauuagcauuuaAAUAGCACAUAUcuuaacaacuaauguuuaguagucaauacagauuaugGAAAUACAU |
| | | ACaaccccuaucacgauuagcauuuaAGAAAUCCAUAUcauaaucguauugauucuuucacuauauacuucaaggACCUCUAACAUaaccccua |
| | | ucacgauuagcauuuaACUAUCCUCUCAccuugaauauauaguucaugcccuguuuauugacaugUCACGCACCGAaaccccuaucacgauuag |
| | | cauuuaUUACAAACCUACcaugucauaaacaguuauacaa |
| x6 sites- | 563 | gggugauauacgaccaguGUCACAUAAAUaaccccuaucacgauuagcauaaaACCGAGAAUAAAacuggucuauaucauuaugagcugauaag |
| Perf | | aaaugacuCCAUCACUUCAaaccccuaucacgauuagcauaaaAAUUCUCAAUCUagucauucuuaucauucagacacauuaguauguuaagC |
| pos3 | | AUUCAACCCUaaccccuaucacgauuagcauaaaUUACGCCUUUCCcuuaacaacuaauguuuaguagucaauacagauuaugAUUAUCCCU |
| | | CUaaccccuaucacgauuagcauaaaCUAUCCAGCCUCcauaaucguauugauucuuucacuauauacuucaaggACCACACCAAUaaccccua |
| | | ucacgauuagcauaaaCCUAAACCAUUAccuugaauauauaguucaugcccuguuuauugacaugAUCUUAACCUCaaccccuaucacgauuag |
| | | cauaaaCAAGACUUUCACcaugucauaaacaguuauacaa |
| x6 sites- | 564 | gggugauauacgaccaguUUUCAGCCUCAaaccccuaucacgauuagcaauaaCUCCUUCCCACCacuggucuauaucauuaugagcugauaag |
| Perf | | aaaugacuCCAACCUCUAAaaccccuaucacgauuagcaauaaCACUCAAACUCUagucauucuuaucauucagacacauuaguauguuaagAG |
| pos4 | | AAACGCCUUaaccccuaucacgauuagcaauaaAUACUGAGCCAAcuuaacaacuaauguuuaguagucaauacagauuaugAACUAACAUU |
| | | AaaccccuaucacgauuagcaauaaUAGCACUCAAGAcauaaucguauugauucuuucacuauauacuucaaggCCUACCUUAACaaccccuau |
| | | cacgauuagcaauaaUACAAUCAAUACccuugaauauauaguucaugcccuguuuauugacaugACCACUCAACCaaccccuaucacgauuagca |
| | | auaaCAAAUCCUACACcaugucauaaacaguuauacaa |
| x6 sites- | 565 | gggugauauacgaccaguCUAACUUACUAaaccccuaucacgauuagcuuuaaAUAUCACAUUCCacuggucuauaucauuaugagcugauaag |
| Perf | | aaaugacuCGAAUUUCCGCaaccccuaucacgauuagcuuuaaCCUAUACCACACagucauucuuaucauucagacacauuaguauguuaagU |
| pos5 | | AAACACUAACaaccccuaucacgauuagcuuuaaCUCACUUUCGACcuuaacaacuaauguuuaguagucaauacagauuaugUCACUUACA |
| | | CUaaccccuaucacgauuagcuuuaaUUUCAUCUACGUcauaaucguauugauucuuucacuauauacuucaaggGAGAUCUUACUaaccccu |
| | | aucacgauuagcuuuaaAUACUCAAUAAGccuugaauauauaguucaugcccuguuuauugacaugUCCACCGCGCAaaccccuaucacgauua |
| | | gcuuuaaAUACAAUCCACCcaugucauaaacaguuauacaa |
| x6 sites- | 566 | gggugauauacgaccaguUUAACAUUUCAaaccccuaucacgauuaggauuaaCAUCACAACCUCacuggucuauaucauuaugagcugauaag |
| Perf | | aaaugacuUAAACUCACUCaaccccuaucacgauuaggauuaaUACCGCUACCCUagucauucuuaucauucagacacauuaguauguuaagCC |
| pos6 | | CAUACAUCAaaccccuaucacgauuaggauuaaAUACACGCCAACcuuaacaacuaauguuuaguagucaauacagauuaugACUCUCACAA |
| | | CaaccccuaucacgauuaggauuaaAGACCCACAACCcauaaucguauugauucuuucacuauauacuucaaggCAAAUAUUAAGaaccccuau |
| | | cacgauuaggauuaaAAAGCUCUCAUCccuugaauauauaguucaugcccuguuuauugacaugCUCUUUAACACaaccccuaucacgauuagg |
| | | auuaaAAACCGCACACUcaugucauaaacaguuauacaa |
| x6 sites- | 567 | gggugauauacgaccaguAUUCUACAACCaaccccuaucacgauuaccauuaaCUAUAGCCUCACacuggucuauaucauuaugagcugauaag |
| Perf | | aaaugacuCAUAAACACAUaaccccuaucacgauuaccauuaaCCAGCAUAACACagucauucuuaucauucagacacauuaguauguuaagCG |
| pos7 | | CUCUACUUUaaccccuaucacgauuaccauuaaCUAACGAAACACcuuaacaacuaauguuuaguagucaauacagauuaugUCAGCCUCCA |
| | | AaaccccuaucacgauuaccauuaaCCCGUAUCAUACcauaaucguauugauucuuucacuauauacuucaaggAGAAAUCUAUCaaccccuau |
| | | cacgauuaccauuaaCCAUAAGCACAAccuugaauauauaguucaugcccuguuuauugacaugACCAUCACAUGaaccccuaucacgauuacca |
| | | uuaaACACGACCCACAcaugucauaaacaguuauacaa |
| x6 sites- | 568 | gggugauauacgaccaguAGACUCCACGAaaccccuaucacgauuugcauuaaCAUACACUUAACacuggucuauaucauuaugagcugauaag |
| Perf | | aaaugacuGAACUACAUAUaaccccuaucacgauuugcauuaaAUACACCCACUAagucauucuuaucauucagacacauuaguauguuaagU |
| pos8 | | AACCUUACGAaaccccuaucacgauuugcauuaaUAAACAAUAUACcuuaacaacuaauguuuaguagucaauacagauuaugCCAUAACUC |
| | | CGaaccccuaucacgauuugcauuaaUACUAUUCAGUCcauaaucguauugauucuuucacuauauacuucaaggACAACCUCCCAaaccccua |
| | | ucacgauuugcauuaaCCACUCCAUAUAccuugaauauauaguucaugcccuguuuauugacaugAAGAACCAAGAaaccccuaucacgauuug |
| | | cauuaaCCACAAUAUAGCcaugucauaaacaguuauacaa |
| x6 sites- | 586 | gggUGAUAUACGACCAGUgcgcacuacacAACCCCUAUCACGUAAGCAUUAAgaaacucaagauACUGGUCUAUAUCAuuaugagcU |
| Imperf | | GAUAAGAAAUGACUauccgaacuacAACCCCUAUCACGUAAGCAUUAAucauccaacaugAGUCAUUCUUAUCAuucagacaCAUU |
| ctrl | | AGUAUGUUAAGugacucaucauAACCCCUAUCACGUAAGCAUUAAcuacucaacgauCUUAACAACUAAUGuuuaguagUCAAUAC |
| | | AGAUUAUGacagcaucuauAACCCCUAUCACGUAAGCAUUAAuacaccacgacgCAUAAUCGUAUUGAuucuuucaCUAUAUACUU |
| | | CAAGGaccaucacuacAACCCCUAUCACGUAAGCAUUAAcacuacuagcagCCUUGAAUAUAUAGuucaugccCUGUUUAUUGACA |
| | | UGucacaucucacAACCCCUAUCACGUAAGCAUUAAcacaucacgcauCAUGUCAUAAACAGuuauacaa |
| x6 sites- | 307 | gggUGAUAUACGACCAGUgcgcacuacacAACCCCUAUCACGUAAGCaAUUAAgaaacucaagauACUGGUCUAUAUCAuuaugagc |
| Imperf | | UGAUAAGAAAUGACUauccgaacuacAACCCCUAUCACGUAAGCaAUUAAucauccaacaugAGUCAUUCUUAUCAuucagacaCAU |
| A | | UAGUAUGUUAAGugacucaucauAACCCCUAUCACGUAAGCaAUUAAcuacucaacgauCUUAACAACUAAUGuuuaguagUCAAU |
| | | ACAGAUUAUGacagcaucuauAACCCCUAUCACGUAAGCaAUUAAuacaccacgacgCAUAAUCGUAUUGAuucuuucaCUAUAUA |
| | | CUUCAAGGaccaucacuacAACCCCUAUCACGUAAGCaAUUAAcacuacuagcagCCUUGAAUAUAUAGuucaugccCUGUUUAUU |
| | | GACAUGucacaucucacAACCCCUAUCACGUAAGCaAUUAAcacaucacgcauCAUGUCAUAAACAGuuauacaa |
| x6 sites- | 308 | gggUGAUAUACGACCAGUucagacuacacAACCCCUAUCACGUAAGCgAUUAAgaaacucaauauACUGGUCUAUAUCAuuaugagc |
| Imperf | | UGAUAAGAAAUGACUacaccgacauaAACCCCUAUCACGUAAGCgAUUAAccuacagagauuAGUCAUUCUUAUCAuucagacaCAU |
| G | | UAGUAUGUUAAGugacucagcauAACCCCUAUCACGUAAGCgAUUAAauucagaacaauCUUAACAACUAAUGuuuaguagUCAAU |
| | | ACAGAUUAUGacagcaacuauAACCCCUAUCACGUAAGCgAUUAAuacaccacgaauCAUAAUCGUAUUGAuucuuucaCUAUAUA |
| | | CUUCAAGGacauaccuaucAACCCCUAUCACGUAAGCgAUUAAcaccaucaccagCCUUGAAUAUAUAGuucaugccCUGUUUAUU |
| | | GACAUGucacaucucacAACCCCUAUCACGUAAGCgAUUAAcacuccacucauCAUGUCAUAAACAGuuauacaa |
| x6 sites- | 309 | gggUGAUAUACGACCAGUgcgcacuacacAACCCCUAUCACGUAAGCcAUUAAgaaacucaagauACUGGUCUAUAUCAuuaugagc |
| Imperf | | UGAUAAGAAAUGACUauccgaacuacAACCCCUAUCACGUAAGCcAUUAAucauccaacaugAGUCAUUCUUAUCAuucagacaCAU |
| C | | UAGUAUGUUAAGugacucaucauAACCCCUAUCACGUAAGCcAUUAAcuacucaacgauCUUAACAACUAAUGuuuaguagUCAAU |
| | | ACAGAUUAUGacagcaucuauAACCCCUAUCACGUAAGCcAUUAAuacaccacgacgCAUAAUCGUAUUGAuucuuucaCUAUAUA |
| | | CUUCAAGGaccaucacuacAACCCCUAUCACGUAAGCcAUUAAcacuacuagcagCCUUGAAUAUAUAGuucaugccCUGUUUAUU |
| | | GACAUGucacaucucacAACCCCUAUCACGUAAGCcAUUAAcacaucacgcauCAUGUCAUAAACAGuuauacaa |
| x6 sites- | 310 | gggUGAUAUACGACCAGUgcgcacuacacAACCCCUAUCACGUAAGCuAUUAAgaaacucaagauACUGGUCUAUAUCAuuaugagc |
| Imperf | | UGAUAAGAAAUGACUauccgaacuacAACCCCUAUCACGUAAGCuAUUAAucauccaacaugAGUCAUUCUUAUCAuucagacaCA |
| U | | UUAGUAUGUUAAGugacucaucauAACCCCUAUCACGUAAGCuAUUAAcuacucaacgauCUUAACAACUAAUGuuuaguagUCAA |
| | | UACAGAUUAUGacaccaucuauAACCCCUAUCACGUAAGCuAUUAAuacaccacgacgCAUAAUCGUAUUGAuucuuucaCUAUAU |
| | | ACUUCAAGGaccaucacaacAACCCCUAUCACGUAAGCuAUUAAcacgaccacgagCCUUGAAUAUAUAGuucaugccCUGUUUAUU |
| | | GACAUGucacaucucacAACCCCUAUCACGUAAGCuAUUAAcacaucacgcauCAUGUCAUAAACAGuuauacaa |
| x6 sites- | 569 | gggugauauacgaccaguACCAUCUCCGCaaccccuaucacguaagcauuuaAAGUUCCCAGCCacuggucuauaucaAACACCAAugauaag |
| Imperf | | aaaugacuUUACAUUAACGaaccccuaucacguaagcauuuaAUAUAAUUACUUagucauucuuaucaGAAACUACcauuaguauguuaag |
| pos2 | | CACUUAAACCAaaccccuaucacguaagcauuuaCCAAAUCGACUAcuuaacaacuaaugCCAACUUAucaauacagauuaugCCCUAGAC |
| | | CAUaaccccuaucacguaagcauuuaACUAUAACCCACcauaaucguauugaACGUUCCCcuauauacuucaaggUAAAUUCCAAGaacccc |
| | | uaucacguaagcauuuaGCCAGAUCAACUccuugaauauauagCGCUCUUCcuguuuauugacaugUAAUACCGACUaaccccuaucacgua |
| | | agcauuuaUUUAUUCACCGCcaugucauaaacagCAUACCUU |
| x6 sites- | 570 | gggugauauacgaccaguCCUCAAUCGCAaaccccuaucacguaagcauaaaCCUAAACAUACUacuggucuauaucaCAAUUAACugauaag |
| Imperf | | aaaugacuCGCCAUAACAGaaccccuaucacguaagcauaaaGAAACUUCCAUUagucauucuuaucaACCUGAACcauuaguauguuaagC |
| pos3 | | AACUCCAAUAaaccccuaucacguaagcauaaaCAAGACCGACCAcuuaacaacuaaugAGCCGCAUucaauacagauuaugAACGCACCA |
| | | AUaaccccuaucacguaagcauaaaAAUAUCAACACCcauaaucguauugaUCAAUCAUcuauauacuucaaggACACCCACAGCaaccccua |
| | | ucacguaagcauaaaGAAAUUCGACCAccuugaauauauagCUACGUACcuguuuauugacaugCAAGAACGCCCaaccccuaucacguaagc |
| | | auaaaUAGAACUUAAGAcaugucauaaacagUCUCCGAG |
| x6 sites- | 571 | gggugauauacgaccaguAAACCAACCUAaaccccuaucacguaagcaauaaGCCAUUUCCACCacuggucuauaucaGAAACCACugauaag |
| Imperf | | aaaugacuAUCCAUCAGUCaaccccuaucacguaagcaauaaCACCAUUAAGCCagucauucuuaucaAUCUUCACcauuaguauguuaagA |
| pos4 | | UUCCGCAGUCaaccccuaucacguaagcaauaaCAUCUUAACUUCcuuaacaacuaaugCUACUUCCucaauacagauuaugCAAUUCGCA |
| | | GAaaccccuaucacguaagcaauaaAAGACAUACCAAcauaaucguauugaUUCAAAUCcuauauacuucaaggCUCAAUUUAAGaaccccu |
| | | aucacguaagcaauaaACAUAUUCACUUccuugaauauauagAGUCAUCUcuguuuauugacaugAUUAAUCCGCAaaccccuaucacguaa |
| | | gcaauaaCUCCCAGAAUUAcaugucauaaacagCUACCUUA |
| x6 sites- | 572 | gggugauauacgaccaguCGACAUCUACUaaccccuaucacguaagcuuuaaUAAUCUACCUAAacuggucuauaucaAUCAUUCCugauaa |
| Imperf | | gaaaugacuCCUCCCAUUAAaaccccuaucacguaagcuuuaaAUUAGACAUAACagucauucuuaucaAUACCUACcauuaguauguuaag |
| pos5 | | CAUCCUAUACUaaccccuaucacguaagcuuuaaCCUUCUGUACCUcuuaacaacuaaugAACUACCCucaauacagauuaugCCACGACU |
| | | AACaaccccuaucacguaagcuuuaaUUUCCACAUCUAcauaaucguauugaAACACAACcuauauacuucaaggAUACAAACCGCaaccccu |
| | | aucacguaagcuuuaaAUGACCCACCGCccuugaauauauagCAGAAACAcuguuuauugacaugCAUAAAUUCUUaaccccuaucacguaa |
| | | gcuuuaaACUCACCAUUAAcaugucauaaacagAAUAGCUA |
| x6 sites- | 573 | gggugauauacgaccaguCUCACUUAAACaaccccuaucacguaaggauuaaAGACACCGCACAacuggucuauaucaUGCUAAACugauaag |
| Imperf | | aaaugacuGCCUUUAACACaaccccuaucacguaaggauuaaUAACGCCCAAUGagucauucuuaucaCCUUUGUUcauuaguauguuaag |
| pos6 | | ACCCAAGAUCAaaccccuaucacguaaggauuaaAUAACGACAAACcuuaacaacuaaugCCACCUGUucaauacagauuaugACCCACAU |
| | | UCAaaccccuaucacguaaggauuaaACACGCCUCAAAcauaaucguauugaUUCCAAUAcuauauacuucaaggAUACAGAACACaacccc |
| | | uaucacguaaggauuaaUUUAGCACACGAccuugaauauauagAUUAUCCCcuguuuauugacaugCACAAACUACAaaccccuaucacgua |
| | | aggauuaaUACACAUAACAUcaugucauaaacagCCUCAAAC |
| x6 sites- | 574 | gggugauauacgaccaguCCAACACCCGAaaccccuaucacguaaccauuaaGCAUUCCGAUUCacuggucuauaucaAUCUCACCugauaag |
| Imperf | | aaaugacuAAAUCCAUUAUaaccccuaucacguaaccauuaaCUCCUGUCUAGAagucauucuuaucaAACUAAUCcauuaguauguuaag |
| pos7 | | AAAUCACUAUAaaccccuaucacguaaccauuaaUUAAUCAAUCACcuuaacaacuaaugAUUCAACCucaauacagauuaugCAUACUCU |
| | | CCCaaccccuaucacguaaccauuaaGAUCGCAAAUACcauaaucguauugaAACUGCAAcuauauacuucaaggACCCGUCUCCUaaccccu |
| | | aucacguaaccauuaaCCAACCCUGUACccuugaauauauagCAAAUAGCcuguuuauugacaugCCACACCAAGAaaccccuaucacguaac |
| | | cauuaaACUAGAACGAAAcaugucauaaacagUACAUACA |
| x6 sites- | 575 | gggugauauacgaccaguACUCCAAUCAAaaccccuaucacguaugcauuaaCUACUUACUUCAacuggucuauaucaUCUCUACCugauaag |
| Imperf | | aaaugacuACACGCAAUCAaaccccuaucacguaugcauuaaAUCACAACCCUCagucauucuuaucaCACAUUCCcauuaguauguuaagA |
| pos8 | | AUUUCUCUACaaccccuaucacguaugcauuaaUCAAACUAAUCGcuuaacaacuaaugAAUCAAUUucaauacagauuaugAGACUUAU |
| | | AUUaaccccuaucacguaugcauuaaUCUUCGCGCCAAcauaaucguauugaCAAUCCUAcuauauacuucaaggGCAAUCCUUCUaacccc |
| | | uaucacguaugcauuaaCUCAAGCUAAGAccuugaauauauagCCUUAUUUcuguuuauugacaugCUAUCAGCUUCaaccccuaucacgu |
| | | augcauuaaCACUUUCUCAUUcaugucauaaacagUCAACGUU |
| x3 sites- | 576 | gggugauauacgaccaguGACGAACCCCUAUCACGUAAGCAUUAAAUAAacuggucuauaucaCAAGACUAugauaagaaaugacuGAA |
| 4/4 | | GAACCCCUAUCACGUAAGCAUUAACCUAagucauucuuaucaGCUAUUUCcauuaguauguuaagAGAAAACCCCUAUCACGUAAG |
| aux arms | | CAUUAAAUGAcuuaacaacuaaugAAUUACAA |
| x3 sites- | 577 | gggugauauacgaccaguUUUCCAAACCCCUAUCACGUAAGCAUUAAGUAUCUacuggucuauaucaUCGAUGAAugauaagaaaugac |
| 6/6 | | uAUCCCUAACCCCUAUCACGUAAGCAUUAAAUGUAAagucauucuuaucaGCACCUCCcauuaguauguuaagAAAUUAAACCCCU |
| aux arms | | AUCACGUAAGCAUUAACCUUAAcuuaacaacuaaugUACAACCA |
| x3 sites- | 578 | gggugauauacgaccaguAAACUUAUAACCCCUAUCACGUAAGCAUUAAAAUCCAAAacuggucuauaucaUACCCAUGugauaagaaa |
| 8/8 | | ugacuACCUAUACAACCCCUAUCACGUAAGCAUUAAACCAGCACagucauucuuaucaAUCAUUACcauuaguauguuaagGCAACA |
| aux arms | | UAAACCCCUAUCACGUAAGCAUUAAAAUCUCAAcuuaacaacuaaugACUCAUCC |
| x3 sites- | 579 | gggucaauacagauuaugUUCCUCAAAUCUAACCCCUAUCACGUAAGCAUUAAGUCCUUAAAGUCcauaaucguauugaCAACUUC |
| 12/12 | | CcuauauacuucaaggCCCUAACUUCGCAACCCCUAUCACGUAAGCAUUAAUACUCCACACCCccuugaauauauagUCAUAAAUcu |
| aux arms | | guuuauugacaugACUCUUGUUCUCAACCCCUAUCACGUAAGCAUUAAUUCCAUCUUUCCcaugucauaaacagCCUCCAUC |
| x6 sites- | 580 | gggugauauacgaccaguGACGAACCCCUAUCACGUAAGCAUUAAAUAAacuggucuauaucaCAAGACUAugauaagaaaugacuGAA |
| 4/4 | | GAACCCCUAUCACGUAAGCAUUAACCUAagucauucuuaucaGCUAUUUCcauuaguauguuaagAGAAAACCCCUAUCACGUAAG |
| aux arms | | CAUUAAAUGAcuuaacaacuaaugAAUUACAAucaauacagauuaugACUCAACCCCUAUCACGUAAGCAUUAAAACAcauaaucgua |
| | | uugaCAUACCUUcuauauacuucaaggGAAAAACCCCUAUCACGUAAGCAUUAAUAGAccuugaauauauagCCGAUUUCcuguuuau |
| | | ugacaugACCCAACCCCUAUCACGUAAGCAUUAACCUCcaugucauaaacagUUAUACCG |
| x6 sites- | 581 | gggugauauacgaccaguAAUUUCAACCCCUAUCACGUAAGCAUUAAACACCCacuggucuauaucaAACUUUAAugauaagaaaugac |
| 6/6 | | uACUCCGAACCCCUAUCACGUAAGCAUUAAUAUUCAagucauucuuaucaAACUCAUCcauuaguauguuaagUCGUCUAACCCCU |
| aux arms | | AUCACGUAAGCAUUAACAAAUCcuuaacaacuaaugUUCAUGCCucaauacagauuaugGAGCCCAACCCCUAUCACGUAAGCAUU |
| | | AAAAAGAGcauaaucguauugaUUUCGAACcuauauacuucaaggCUAUACAACCCCUAUCACGUAAGCAUUAAGAACUAccuugaa |
| | | uauauagAUCAAUUAcuguuuauugacaugACCAGCAACCCCUAUCACGUAAGCAUUAACGACUCcaugucauaaacagACUAACCA |
| x6 sites- | 582 | gggugauauacgaccaguAAACUUAUAACCCCUAUCACGUAAGCAUUAAAAUCCAAAacuggucuauaucaUACCCAUGugauaagaaa |
| 8/8 | | ugacuACCUAUACAACCCCUAUCACGUAAGCAUUAAACCAGCACagucauucuuaucaAUCAUUACcauuaguauguuaagGCAACA |
| aux arms | | UAAACCCCUAUCACGUAAGCAUUAAAAUCUCAAcuuaacaacuaaugACUCAUCCucaauacagauuaugACUGACCGAACCCCUAU |
| | | CACGUAAGCAUUAACUAUAUUCcauaaucguauugaACUUCACAcuauauacuucaaggAACCUACUAACCCCUAUCACGUAAGCA |
| | | UUAACUGAACUCccuugaauauauagCCGUUCACcuguuuauugacaugGUCAGUCCAACCCCUAUCACGUAAGCAUUAAUAUCG |
| | | UUAcaugucauaaacagUUAGUUAG |
| x6 sites- | 583 | gggugauauacgaccagucacuccuuaacaaaccccuaucacguaagcauuaauauaccugcccuacuggucuauaucaguaccugcugauaagaaauga |
| 12/12 | | cuuccacaaacagcaaccccuaucacguaagcauuaaaccucacaucucagucauucuuaucaccuuuccucauuaguauguuaagauagccaucaccaa |
| aux arms | | ccccuaucacguaagcauuaagagaaucauaaacuuaacaacuaaugcugucauaucaauacagauuauguuccucaaaucuaaccccuaucacguaagc |
| | | auuaaguccuuaaaguccauaaucguauugacaacuucccuauauacuucaaggcccuaacuucgcaaccccuaucacguaagcauuaauacuccacacc |
| | | cccuugaauauauagucauaaaucuguuuauugacaugacucuuguucucaaccccuaucacguaagcauuaauuccaucuuucccaugucauaaacag |
| | | ccuccauc |

In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the sequence in Table 9 or Table 10. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 433-511 or 512-583. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 435, 438, 514, or 517. In some embodiments, an engineered nucleic acid herein can comprise a nucleic acid sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO: 441, 449, 506, 521, 528, or 578. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NO: 433-511 or 512-583. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NO: 435, 438, 514, or 517. In some embodiments, an engineered nucleic acid herein can comprise a nucleic acid sequence with about: 50%, 60%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence length to any one of SEQ ID NO: 441, 449, 506, 521, 528, or 578.

### Nucleic Acid Targets

In some aspects, an engineered nucleic acid can act as a sponge to sequester a nucleic acid. In some aspects, an engineered nucleic acid can sequester a microRNA (miRNA). In some instances, an engineered nucleic acid can act as a sponge to sequester RNA that has not undergone post RNA transcription processing such as splicing out introns, for example a pre-RNA or pre-mRNA. In some instances, an engineered nucleic acid can sequester mRNA. In some instances, an engineered nucleic acid can sequester a ribozyme. In some aspects, an engineered nucleic acid can act as a sponge to sequester a mRNA. In some instances, an engineered nucleic acid can sequester a single stranded DNA molecule. In some cases, an engineered nucleic acid can sequester a messenger RNA (mRNA), a ribosomal RNA (rRNA), a transfer RNA (tRNA), a ribozyme, a transfer messenger RNA (tmRNA), a double stranded RNA (dsRNA), a small nuclear RNA (ssRNA), a small nucleolar RNA (snoRNA), a Piwi-interacting RNAs (piRNA), a microRNA (miRNA), a small interfering RNA (siRNA), a long non-coding RNA (lncRNA), or any combination thereof.

In instances wherein sequestration of a miRNA is contemplated, an engineered nucleic acid may be generated to sequester any miRNA conferring a detriment to a cell, a benefit to a cell, a function of a cell, a growth of a cell, a differentiation of a cell, or a miRNA which inhibits or activates a reporter protein in a cell after translation. A non-limiting list of target miRNAs and their disease indication are listed in Table 1 and Table 2. Administration of an engineered nucleic acid herein can be used to treat any of the diseases is Table 1 or Table 2.

TABLE 1
List of Diseases and Potential Target
miRNAs Indicated in the Disease

| Disease | Disease type | miRNA type |
|---|---|---|
| Cancer | B-CLL | miR-15, miR-16 |
| | Breast cancer | miR-125b, miR-145, miR- |
| | | 21, miR-155, miR-210 |
| | Lung cancer | miR-155, let-7a |
| | Gastric cancer | miR-145 |
| | Liver cancer | miR-29b |
| Viral diseases | HCV | miR-122, miR-155 |
| | HIV-1 | miR-28, miR-125b, miR- |
| | | 150, miR-223, miR-382 |
| | Influenza | miR-21, miR-223 |
| Immune related | Multiple sclerosis | miR-145, miR-34a, miR- |
| diseases | | 155, miR-326 |
| | Systemic lupus | miR-146a |
| | erythematosus | |
| | Type II diabetes | miR-144, miR-146a, miR- |
| | | 150, miR-182, miR-103, |
| | | miR-107 |
| | Non-alcoholic fatty | miR-200a, miR-200b, |
| | liver disease | miR-429, miR-122, miR- |
| | | 451, miR-27 |
| | Non-alcoholic | miR-29c, miR-34a, miR- |
| | steatohepatitis | 155, miR-200b |
| Neurodegenerative | Parkinson's disease | miR-30b, miR-30c, miR- |
| diseases | | 26a, miR-133b, miR- |
| | | 184*, let-7 |
| | Alzheimer's disease | miR-29b-1, miR-29a, |
| | | miR-9 |

TABLE 2
List of Target miRNAs Indicated in Different Diseases

| miRNA | |
|---|---|
| Candidates | Disease Indication |
| miR-21-3p | Proliferative vitroretinopathy (PVR) |
| miR-21-5p | |
| miR-27a-5p | |
| miR-223-5p | |
| miR-361-5p | |
| miR-128-1 | Metabolic Syndrome, Familial Hypercholesterolemia |
| miR-148a | |
| miR-122 | Hepatitis C virus (HCV) infection |
| miR-103 | Diabetic non-alcoholic steatohepatitis (NASH) |
| miR-107 | |
| miR-21 | Alport Syndrome |
| miR-17 | Autosomal dominant polycystic kidney disease |
| miR-132 | Non-alcoholic steatohepatitis (NASH) |
| miR-132 | Stable ischemic heart failure |
| miR-212 | Cardiac hypertrophy and hear failure |
| miR-1 | |
| miR-25 | |
| miR-208 | |
| miR-155 | Cutaneous T-cell Lymphoma (CTCL) |
| | Adult T-cell Lymphoma (Leukemia) |
| | Atopic dermatitis |
| | Intersitial lung disease |
| miR-92a | Chronic ischemic disorders |
| miR-33 | Atherosclerosis |

### Engineered Nucleic Acid Delivery

In certain aspects of the disclosure, delivery of an engineered nucleic acid to a cell or a subject or a cell of a subject in need thereof is contemplated. In some cases, an engineered nucleic acid may be encoded by a polynucleotide. For example, an engineered nucleic acid can be encoded in a viral vector or can be encoded by DNA in the form of a plasmid or an episome. In certain aspects, delivery of a nucleic acid intended to function as an engineered nucleic acid or to produce an engineered nucleic acid is through liposomal delivery. In certain instances, the liposome may be a positively charged liposome. In certain instances, the liposome may be a negatively charged liposome. In other instances, the delivery of engineered nucleic acid is a polymer delivery. In other instances, the engineered nucleic acid delivery is a dendrimer mediated delivery. In other instances, the engineered nucleic acid delivery is a nanoparticle mediated delivery. In certain aspects, delivery of a nucleic acid intended to be an engineered nucleic acid is through a viral vector. For example, an adeno associated virus (AAV) vector can encode an engineered nucleic acid. An AAV vector can comprise AAV serotype 1, 2, 3, 4, 5, 6, 7, 8, 9, 10 or 11. In some cases, an AAV vector can comprise a mix of serotypes, can be genetically modified, or a combination thereof. In some cases, AAV vectors may infect non-dividing cells. A desired DNA (e.g., a DNA encoding a engineered nucleic acid) may be engineered to be inserted between a 5′ and 3′ inverted terminal repeats (ITRs) of an AAV based vector. In some cases, a desired DNA can be integrated into the nucleus of a target cell. In certain instances, at least part of the AAV genome may integrate into a human chromosome, such as human chromosome 19. In certain instances, the viral vector can be a DNA virus such as a herpes virus. In the case of herpes virus vectors, the virus may be derived from herpes simplex 1 or 2. In certain instances, the herpes vector can have a deletion at the ICP4 locus. In some cases, a DNA encoding an engineered nucleic acid can be inserted into a retrovirus after reverse transcription from RNA to DNA. In some cases, an engineered nucleic acid can comprise the use of an adenovirus, or a pox virus as a vector. In certain instances, in the case of an adenoviral vector, a DNA coding for an engineered RNA, can be typically inserted into the E1A region of the adenoviral vector, rendering the adenovirus unable to replicate while still being able to deliver the engineered nucleic acid. In certain instances, such as in the case of retroviral vectors, and more specifically lentiviral vectors, the vector has a nuclear localization signal. In some cases, a nuclear localization signal can comprise AGCCC, a sequence derived from lncRNAs JPX, PVT1, NR2F1-AS1 Emxos, AluSx, a SIRLOIN element or any combination thereof. In some cases, an engineered nucleic acid herein can comprise a nuclear localization signal. Examples of lentiviruses include SIV, HIV, BIV, and FIV. In lentiviral vectors, the viral vector is designed to be incapable of replication after delivering the payload, in this case a DNA sequence coding for an engineered nucleic acid to a cell. Lentiviral vectors typically contain a nuclear localization signal within the nucleocapsid protein of the virion enabling a non-dividing cell, such as a cardiac tissue cell, to have the DNA encoding the nucleic acid delivered to the nucleus. In other instances, the delivery of an engineered nucleic acid or a DNA encoding an engineered nucleic acid is via microinjection, electroporation, ultrasound, gene gun or hydrodynamic applications. In other instances, the delivery of an engineered nucleic acid is via conjugation to or association with a nanoparticle.

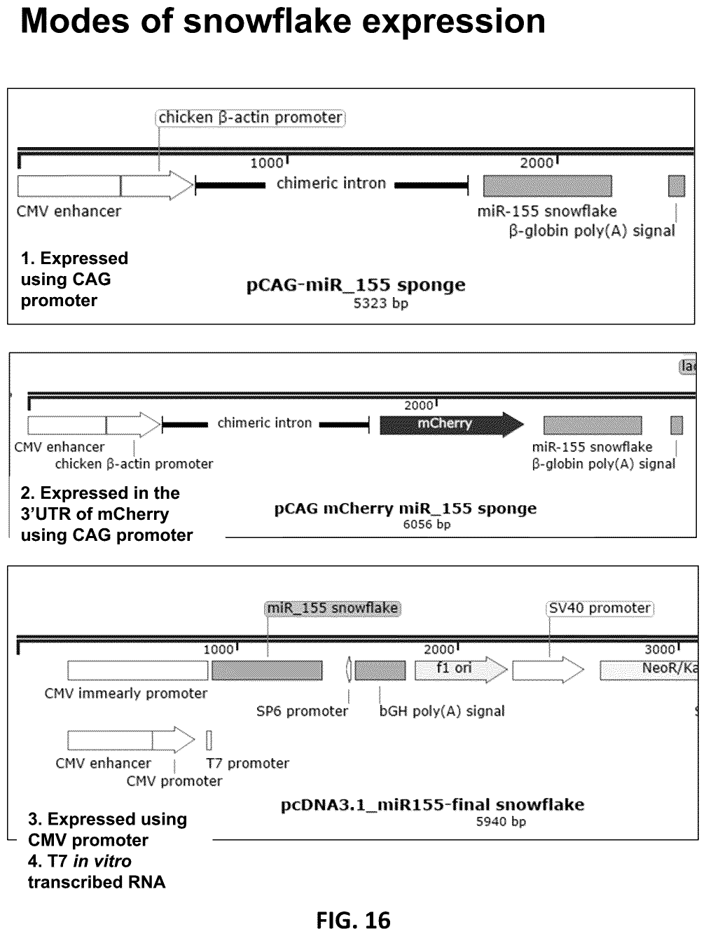

In certain instances, the engineered nucleic acid is delivered as produced. For example, in certain instances, such as with delivery in a liposome or gene gun, a synthetically produced engineered nucleic acid, which is single stranded, can be introduced into a liposome and delivered into a cell and/or subject in need of therapy. A biologically produced engineered nucleic acid, isolated from an engineered nucleic acid production cell, can be introduced into a liposome and delivered to a cell, a subject, a human subject, or a cell of a subject or a human subject, in need of therapy. In other instances, the delivery can be by a gene construct on a plasmid or in a viral vector such that upon transcription within the cell, the engineered nucleic acid is formed—for example as shown in FIG. 16 wherein an engineered nucleic acid encoded DNA can be 1) expressed using a CAG promoter, 2) expressed in the 3′UTR of mCherry using a CAG promoter, and/or 3) expressed using a CMV promoter. In some cases, the engineered nucleic acid encoded DNA may not be expressed in the 3′UTR of mCherry using a CAG promoter. In some cases, a promoter can be used to express an engineered nucleic acid, such as a sfRNA disclosed herein. In some cases, a promoter can be operably linked to a coding region. In some cases, the coding region can comprise a sfRNA. Other examples of promoters can include a CMV promoter, a EF1a promoter, a SV40 promoter, a PGK1 promoter, a Ubc promoter, a CAG promoter, a TRE promoter, a UAS promoter, a U6 promoter, a H1 promoter, or a combination thereof.

### Subjects

In some aspects, a subject can comprise a mammal. In some aspects, a mammal can comprise a mouse, rabbit, pig, cow, dog, primate, or human. In some aspects, a subject can comprise a patient in need thereof. In some cases, a subject can be a human. A human can be at any developmental stage, for example a human can be a fetus, an infant, a child, or an adult. In some cases, a human can be from about: 1 day to about 7 days old, 1 week to about 5 weeks old, 1 month to about 12 months old, 1 year to about 6 years old, 5 years to about 15 years old, 14 years to about 30 years old, 25 years to about 50 years old, 40 years to about 75 years old, 70 years to about 100 years old, 85 years old to about 110 years old or about 100 years to about 130 years old. In some aspects, a subject can be immunocompromised, can have a cancer, can have lupus, can be receiving treatment with a molecule which modulates a subject's immune system, or any combination thereof. In some aspects, the human can be male. In some aspects, the human can be female. In some instances, the human or the subject can be a human or a subject in need thereof.

In some aspects, a subject can be diagnosed with a disease. In some cases, a subject can be diagnosed prior to treating a disease. In some cases, a diagnosis can comprise a physical examination, a biopsy, a radiological image, a blood test, an in vitro diagnostic, or any combination thereof. In some cases, a diagnosis can comprise a colonoscopy. A radiological image can comprise an X-Ray, a computed tomography (CT) scan, a magnetic resonance image (MRI), a mammogram, a positron emission tomography (PET image), an ultrasound or any combination thereof. In some cases, a blood test can comprise a diagnostic test such as an ELISA, or a rapid antibody test. In some cases, a blood test can comprise a blood count, a metabolic panel, a lipid panel, a hormone panel, a liver panel or any combination thereof. A biopsy can comprise a biopsy of tissue, such as breast tissue or liver tissue. In some instances, a physical examination can comprise examination for lumps, or growths on the body and examination of bodily functions such as blood pressure, pulse, and breathing rate.

In some aspects, a subject can be tested for a predisposition towards a disease. In some aspects, testing for a predisposition can comprise reviewing a medical history, genetic testing, or a combination thereof. In some aspects, reviewing a medical history can comprise reviewing comorbidities and pre-existing conditions. In some aspects, a comorbidity or pre-existing condition can increase a risk of infection or risk of a severe symptom. In some aspects a genetic test can comprise high-throughput sequencing, cell-free nucleic acid sequencing, or any combination thereof. In some aspects, a genetic test can determine a genetic predisposition towards developing a cancer. In some aspects, a genetic test can determine a genetic predisposition towards developing a cancer.

### Diseases and Conditions

Disclosed herein are methods of compositions for use in treating or preventing a disease or condition. Also disclosed herein in some aspects, are methods and compositions of treating, preventing, or ameliorating a symptom of a disease, such as a heart disease. In some instances, a disease can comprise a cardiac disease, a neurological disease, a cancer, or a viral disease.

In some aspects, a disease can comprise an infection or disease. In some aspects, a disease can comprise a cancer. In some cases, an infection can comprise a bacterial, viral, or fungal infection or disease associated with a virus, bacteria, or fungus.

In certain instances, the engineered nucleic acid is designed or configured to sequester a miR-155. In certain instances, sequestration or sponging of a miR-155 reduces the endogenous expression of angiotensin 2 reception AT1R protein. AT1R is thought to mediate angiotensin 2 related elevation on blood pressure which may contribute to the pathogenesis of heart failure. In other instances, miR-155 may result in triggering oncogenic cascades that begin by apoptotic resistance. In certain instances, a P53 induced nuclear protein 1 (TP53INP1) can be silenced by miR-155. In certain instances, the engineered nucleic acid is designed to sequester a miR-17, which may be overexpressed in a wide variety of cancer types. In certain instances, the engineered nucleic acid is designed to sequester a miR-132. Expression of miR-132 may result in proliferation of endothelial cells and neovascularization and thus may play a role in angiogenesis. In the case of cardiac disease, Angiotensin 2 receptor type 1 mRNA can be silenced by a miR-132. In certain instances, the engineered nucleic acid can be designed to sequester a miR-18. Expression of a miR-18 may result in downregulation of interferon regulatory factor 2 (IRF2). IRF2 can increase apoptosis, inhibit cell proliferation and migration ability.

### Cardiac Diseases

In some cases, a disease can be a disease of the heart. In certain instances, the disease is hypertension, a metabolic syndrome, a valve disease, cardiac hypertrophy, cardiac hypotrophy, cardiac fibrotic remodeling, cardiac wall stiffness, stable angina, unstable angina, variant angina, atrial fibrillation, hart block, premature atrial complex, atrial flutter, paroxysmal supraventricular tachycardia, Wolff-Parkinson-White syndrome, premature ventricular complex, ventricular tachycardia, ventricular fibrillation, long QT syndrome, dilated cardiomyopathy, hypertrophic cardiomyopathy, restrictive cardiomyopathy, congestive heart failure, arterial septal defect, ventricular septal defect, patent ductus arteriosus, pulmonic stenosis, congenital aortic stenosis, coarctation of aorta, tetralogy of Fallot, tricuspid atresia, truncus arteriosus, Ebstein's anomaly of the tricuspid valve, cor pulmonale, myocardial infarction, mitral stenosis, mitral valve regurgitation, mitral valve prolapse, aortic stenosis, aortic regurgitation, tricuspid stenosis, tricuspid regurgitation, myocarditis, pericarditis, rheumatic heart disease, cardiac tumor, aortic aneurysm, arteriosclerosis, atherosclerosis, aortic dissection, hypertension, transient ischemic attack, and other cardiac related diseases, and any combination thereof.

### Viral Diseases

In some aspects, a disease can be caused by a viral infection. In some aspects, a viral infection can be caused by a virus of the realm Riboviria. In some aspects, a viral infection can be caused by a virus of the Phylum Incertae sedis. In some aspects, a viral infection can be caused by a virus of the Order Nidovirales. In some aspects, a viral infection can be caused by a virus of the Family Coronaviridae. In some aspects, a viral infection can be caused by a virus of the Subfamily Coronavirinae. In some aspects, a viral infection can be caused by a virus of the Genus Betacoronavirus. In some aspects, a viral infection can be caused by a virus of the Subgenus Sarbecovirus. In some aspects, a viral infection can be caused by a virus of the Species Severe acute respiratory syndrome-related coronavirus. In some aspects, a viral infection can be caused by a virus of the strain Severe Acute Respiratory Syndrome Coronavirus 2 (SARS-COV-2). In some aspects a virus can comprise a mutant derived from the species Severe acute respiratory syndrome-related coronavirus.

In some cases, the infection is caused by an influenza virus such as H1N1, H3N2, H7N9, or H5N1 influenza strains. In aspects, an infection can comprise an infection from adenovirus such as adenovirus B, adenovirus C, or adenovirus E. In some cases, the infection is caused by a metapneumo virus, a parainfluenza virus 1, parainfluenza virus 2, parainfluenza virus 3, coronavirus OC43, coronavirus NL63, coronavirus MERS, coronavirus HKU1, coronavirus 229E, bocavirus type 2,4, and bocavirus type 1,3. In some cases, an infection can comprise an infection from a herpes virus, herpes simplex virus (HSV) such as HHV 1, HHV 2, HHV 3, HHV 4, HHV 5, HHV 6, HHV 7, and HHV 8. In some cases, the infection is caused by a retrovirus such as HIV-1, HIV-2, HTLV-1, or HTLV-2. In some cases, the disease results in hepatitis such as a disease from hepatitis B, hepatitis delta, or hepatitis c. Other viral diseases include diseases derived from infection with Nile Virus, Nipah virus, Hendra virus, a paramyxovirus, Epstein-Barr Virus, a dengue Virus, a rhabdovirus such as rabies, a picornavirus such as polio, a filovirus such as Ebola or Marburg, or a poxvirus such as monkeypox.

### HIV-1

HIV-1 virus is a positive stranded RNA retrovirus that is the causative agent of AIDS. Among the numerous proteins encoded by the HIV-1 genome, as well as that of HIV-2 and SIV, is the Tat protein which can enhance the efficiency of viral transcription. One potential target of a therapy against HIV-1 may be the downregulation of an RNA encoding the Tat protein.

Potential binding sites between an engineered nucleic acid of the present disclosure may include complementary binding between a portion of the Tat RNA and at least one binding site of an engineered nucleic acid herein. The binding site may be 70% complementary to a sequence found in an RNA encoding the Tat protein. In other instances, the binding site may exhibit over: 70 percent, 80 percent, 85 percent, 90 percent, 95 percent or more complementary binding between a binding site of an engineered nucleic acid and a target sequence of an RNA encoding a Tat protein. In some cases, the binding site may exhibit perfect complementary binding between the engineered nucleic acid and the target sequence. A DNA sequence corresponding to the Tat RNA of a HIV-1 virus is found on GenBank with accession number NC_001802 and is shown below in Table 3 (SEQ. ID. NO 1). An example of an RNA binding site with a reverse compliment to a portion of the RNA encoding Tat is found in Table 3 (SEQ ID NO: 2). It should be noted that the binding sites may not be exclusive to those listed.

### Dengue Virus

Dengue virus is a positive stranded RNA flavivurus that results in dengue fever. Symptoms may include a high fever, headache, vomiting, muscle and joint pains, and a skin rash. In a small proportion of cases, dengue fever develops into dengue hemorrhagic fever. One potential target of a therapy against dengue fever may be the NS4B gene of Dengue virus. This gene encodes a viral membrane protein that may serve as a scaffold for dengue replication complex formation.

An engineered nucleic acid of the present disclosure may be designed to interact with all or a portion of a dengue virus RNA encoding the NS4B protein. Binding sites between an engineered nucleic acid of the present disclosure may include complementary binding between a portion of the NS4B RNA and at least one binding site of an engineered nucleic acid of the present disclosure. The binding site may be 70% complementary to a sequence found in an RNA encoding the NS4B protein. In other instances, the binding site may exhibit at or over: 70 percent, 80 percent, 85 percent, 90 percent, 95 percent or more complementary binding between a binding site of an engineered nucleic acid and a target sequence of an RNA encoding a NS4B protein. In some cases, the binding site may exhibit perfect (e.g., 100%) complementary binding between the engineered nucleic acid and the target sequence. A DNA sequence corresponding to the NS4B RNA of a dengue virus is found on GenBank with accession number JN406515 and is shown below in Table 3 (SEQ. ID. NO 3). An example of an RNA binding site with a reverse compliment to a portion of the RNA encoding NS4B is found in Table 3 (SEQ ID NO: 4). It should be noted that the binding sites may not be exclusive to those listed.

### SARS-COV-2

SARS-COV-2 virus is a positive stranded RNA coronavirus that is the causative agent of COVID-19. Among the numerous proteins encoded by the SARS-COV-2 genome is its RNA dependent RNA Polymerase. One potential target of a therapy against SARS-COV-2 may be the downregulation of an RNA encoding the RNA dependent RNA polymerase.

Potential binding sites between an engineered nucleic acid of the present disclosure may include complementary binding between a portion of the RNA dependent RNA polymerase RNA as mentioned above and at least one binding site of an engineered nucleic acid of the present disclosure. The binding site may be 70% complementary to a sequence found in an RNA encoding the RNA dependent RNA polymerase protein. In other instances, the binding site may exhibit at or over: 70 percent, 80 percent, 85 percent, 90 percent, 95 percent or more complementary binding between a binding site of an engineered nucleic acid and a target sequence of an RNA encoding a RNA dependent RNA polymerase protein. In some cases, the binding site may exhibit perfect complementary binding between the engineered nucleic acid and the target sequence. A DNA sequence corresponding to the RNA dependent RNA polymerase RNA of a SARS-COV-2 virus is found on GenBank with accession number NC_045512.2 and is shown below in Table 3 (SEQ. ID. NO 5). An example of an RNA binding site with a reverse compliment to a portion of the RNA encoding the RNA dependent RNA polymerase is found in Table 3 (SEQ ID NO: 6). It should be noted that the binding sites may not be exclusive to those listed.

### Herpes Virus

Herpes viruses are double stranded DNA viruses. There are nine known human herpes viruses. Among these is HHV4, also known as Epstein-Barr virus. Among the numerous proteins encoded by the Epstein-Barr genome is BMRF1, a DNA polymerase processivity subunit. One potential target of a therapy against Epstein-Barr may be the downregulation of an RNA encoding the BMRF1 protein.

Potential binding sites between an engineered nucleic acid of the present disclosure may include complementary binding between a portion of the BMRF1 RNA as mentioned above and at least one binding site of an engineered nucleic acid of the present disclosure. The binding site may be 70% complementary to a sequence found in an RNA encoding BMRF1 protein. In other instances, the binding site may exhibit at or over: 70 percent, 80 percent, 85 percent, 90 percent, 95 percent or more complementary binding between a binding site of an engineered nucleic acid and a target sequence of an RNA encoding a BMRF1 protein. In some cases, the binding site may exhibit perfect complementary binding between the engineered nucleic acid and the target sequence. A DNA sequence coding for BMRF1 of Epstein-Barr virus is found on GenBank with accession number NC_007605.1 and is shown below in Table 3 (SEQ. ID. NO 7). An example of an RNA binding site with a reverse compliment to a portion of an RNA encoding BMRF1 is found in Table 3 (SEQ ID NO: 8). It should be noted that binding sites may not be exclusive to those listed.

### Influenza H1N1

Influenza H1N1 virus is a segmented negative stranded RNA virus. Among the numerous proteins encoded by the Influenza H1N1 genome is its matrix protein M1. One potential target of a therapy against influenza H1N1 may be the downregulation of an RNA encoding the M1 protein.

Potential binding sites between an engineered nucleic acid of the present disclosure may include complementary binding or hybridization between a portion of the M1 protein RNA as mentioned above and at least one binding site of an engineered nucleic acid of the present disclosure. The binding site may be 70% complementary to a sequence found in an RNA encoding M1 protein. In other instances, the binding site may exhibit at or over: 70 percent, 80 percent, 85 percent, 90 percent, 95 percent or more complementary binding between a binding site of an engineered nucleic acid and a target sequence of an RNA encoding a M1 protein. In some cases, the binding site may exhibit perfect complementary binding between the engineered nucleic acid and the target sequence. A DNA sequence corresponding to the M1 RNA of an influenza H1N1 virus is found on GenBank with accession number CY043484.1 and is shown below in Table 3 (SEQ. ID. NO 9). An example of an RNA binding site with a reverse compliment to a portion of the RNA encoding M1 is found in Table 3 (SEQ ID NO: 10). It should be noted that the binding sites may not be exclusive to those listed.

### Rabies Virus

Rhabdoviruses are single stranded negative sense RNA viruses. Among these is the rabies virus, which has a high mortality rate in humans. Among the numerous proteins encoded by the rabies virus genome is its RNA dependent RNA polymerase. One potential target of a therapy against rabies may be the downregulation of an RNA encoding this protein.

Potential binding sites between an engineered nucleic acid of the present disclosure may include complementary binding between a portion of the RNA encoding the rabies RNA dependent RNA polymerase as mentioned above and at least one binding site of an engineered nucleic acid of the present disclosure. The binding site may be 70% complementary to a sequence found in an RNA encoding RNA dependent RNA polymerase. In other instances, the binding site may exhibit at or over: 70 percent, 80 percent, 85 percent, 90 percent, 95 percent or more complementary binding between a binding site of an engineered nucleic acid and a target sequence of an RNA encoding an RNA dependent RNA polymerase of rabies. In some cases, the binding site may exhibit perfect complementary binding between the engineered nucleic acid and the target sequence. A DNA sequence coding for the RNA dependent RNA polymerase of rabies is found on GenBank with accession number M31046.1 and is shown below in Table 3 (SEQ. ID. NO 11). An example of an RNA binding site with a reverse compliment to a portion of an RNA encoding the RNA dependent RNA polymerase is found in Table 3 (SEQ ID NO: 12). It should be noted that the binding sites may not be exclusive to those listed.

### Smallpox

Pox viruses of the family poxviridae are double stranded DNA viruses. Among these is the smallpox virus. Among the numerous proteins encoded by the smallpox virus genome is its EV maturation protein, which facilitates the transport of intracellular viral particles to the cell membrane. One potential target of a therapy against smallpox may be the downregulation of an RNA encoding this protein.

Potential binding sites between an engineered nucleic acid of the present disclosure may include complementary binding between a portion of the RNA encoding the smallpox EV maturation protein as mentioned above and at least one binding site of an engineered nucleic acid of the present disclosure. The binding site may be 70% complementary to a sequence found in an RNA encoding the smallpox EV protein. In other instances, the binding site may exhibit at or over: 70 percent, 80 percent, 85 percent, 90 percent, 95 percent or more complementary binding between a binding site of an engineered nucleic acid and a target sequence of an RNA the smallpox EV protein. In some cases, the binding site may exhibit perfect complementary binding between the engineered nucleic acid and the target sequence. A DNA sequence coding for the smallpox EV protein is found on GenBank with accession number NC_001611.1 and is shown below in Table 3 (SEQ. ID. NO 13). An example of an RNA binding site with a reverse compliment to a portion of an RNA encoding the EV protein is found in Table 3 (SEQ ID NO: 14). It should be noted that the binding sites may not be exclusive to those listed.

### Anti-Inflammatory Cytokine Storm

COVID-19 disease as a result of SARS-COV-2 is often accompanied by an aggressive inflammatory response with the release of a large amount of pro-inflammatory cytokines in an event known as “cytokine storm.” The host immune response to the SARS-COV-2 virus is hyperactive resulting in an excessive inflammatory reaction.

The CCL2 protein is a cytokine of the CC chemokine family that plays a role in the recruitment of monocytes, memory T cells, and dendritic cells to the sites of inflammation produced by infection. Potential binding sites between an engineered nucleic acid of the present disclosure may include complementary binding between a portion of the CCL2 RNA and at least one binding site of an engineered nucleic acid of the present disclosure. The binding site may be 70% complementary to a sequence found in an RNA encoding the CCL2 protein. In other instances, the binding site may exhibit at or over: 70 percent, 80 percent, 85 percent, 90 percent, 95 percent or more complementary binding between a binding site of an engineered nucleic acid and a target sequence of an RNA encoding the CCL2 protein. In some cases, the binding site may exhibit perfect complementary binding between the engineered nucleic acid and the target sequence. A DNA sequence corresponding to the CCL2 RNA may be found on GenBank with accession number AK311960.1 is shown below in Table 3 (SEQ. ID. NO 15). An example of an RNA binding site with a reverse compliment to a portion of the CCL2 RNA is found in Table 3 (SEQ ID NO: 16). It should be noted that the binding sites may not be exclusive to those listed.

### Cancers

In some cases, a cancer can comprise a sarcoma, a carcinoma, a melanoma, a lymphoma, a leukemia, a blastoma, a germ cell tumor, a myeloma, or any combination thereof. In some cases, a cancer can comprise a non-small cell lung cancer (e.g., non-small cell lung cell carcinoma), a solid tumor, or both. In some cases, a cancer can comprise small cell lung cancer. In some cases, a cancer can comprise a solid tumor, a metastatic tumor. A cancer can be a carcinoma of lung, colorectum, pancreas, larynx, stomach, peripheral and central nervous system including glioblastoma multiforme, head and neck, prostate, mammary, other carcinomas. A cancer can comprise a sarcoma, chronic myeloid leukemia (CML), acute myeloid leukemia (AML), acute lymphatic leukemia (ALL), non-Hodgkin Lymphoma (NHL), myeloproliferative syndrome (MPS), myelodysplastic syndrome (MDS), plasmacytoma, and other leukemias. In some cases, a cancer can comprise a bladder cancer, a breast cancer, a colorectal cancer, a kidney cancer, a liver cancer, a lung cancer, a lymphoma, a skin cancer, a melanoma, an oral cancer, an oropharyngeal cancer, a pancreatic cancer, a prostate cancer, a thyroid cancer, a uterine cancer, an adenoid cystic carcinoma, an adrenal gland tumor, anal cancer, appendix cancer, a childhood cancer, an astrocytoma, a bone cancer, a brain tumor, a cervical cancer, a desmoid tumor, an ependymoma, an esophageal cancer, an eye cancer, an eyelid cancer, a familial cancer (e.g. adenomatous polyposis, familial GIST, malignant melanoma, pancreatic cancer), a gall bladder cancer, a gastrointestinal stromal tumor, a head and neck cancer, a hereditary cancer, a leukemia (e.g. acute lymphoblastic, acute lymphocytic, acute myeloid, B-cell prolymphocytic leukemia, chronic lymphocytic, chronic myeloid chronic T-cell lymphocytic, eosinophilic), Li-Fraumeni Syndrome, Lymphoma (e.g. Hodgkin, non-Hodgkin, childhood, lynch syndrome or any combination thereof. In some cases, a cancer can comprise a mastocytosis, a medulloblastoma, a meningioma, a mesothelioma, a endocrine neoplasia, a neuroblastoma, a neuroendocrine tumor, a neurofibromatosis, a osteosarcoma, a parathyroid cancer, a penile cancer, Peutz-Jeghers Syndrome, a pheochromocytoma, a retinoblastoma, a rhabdomyosarcoma, a salivary gland cancer, Kaposi sarcoma, a soft tissue sarcoma, a stomach cancer, a testicular cancer, a thymoma carcinoma, a thyroid cancer, a uterine cancer, Von Hippel-Lindau Syndrome, a vulvar cancer, Waldenstrom Macroglobulinemia, Werner Syndrome, Wilms Tumor, xeroderma pigmentosum, or any combination thereof.

### N-myc

N-myc proto-oncogene protein also known as N-Myc or basic helix-loop-helix protein 37 (bHLHe37), is a protein that in humans is encoded by the MYCN gene. Amplification and overexpression of N-Myc can lead to tumorigenesis. Excess N-Myc is associated with a variety of tumors, most notably neuroblastomas where patients with amplification of the N-Myc gene tend to have poor outcomes.

An engineered RNA of the present disclosure may help to downregulate N-myc by complementary binding between a portion of an mRNA encoding N-myc and a binding site of an engineered RNA. The binding site may be 70% complementary to a sequence found in an RNA encoding the N-myc protein. In other instances, the binding site may exhibit at or over: 70 percent, 80 percent, 85 percent, 90 percent, 95 percent or more complementary binding between a binding site of an engineered nucleic acid and a target sequence of an RNA encoding the N-myc protein. In some cases, the binding site may exhibit perfect complementary binding between the engineered nucleic acid and the target sequence. A DNA sequence corresponding to the N-myc RNA may be found on GenBank with accession number NM_001293228.2 is shown below in Table 3 (SEQ. ID. NO 17). An example of a binding site with a reverse compliment to a portion of the N-myc RNA is found in Table 3 (SEQ ID NO: 18). It should be noted that the binding sites may not be exclusive to those listed.

### Survivin

Survivin is a member of the inhibitor of apoptosis (IAP) family. The survivin protein functions to inhibit caspase activation, thereby leading to negative regulation of apoptosis or programmed cell death. This has been shown by disruption of survivin induction pathways leading to increase in apoptosis and decrease in tumor growth.

An engineered RNA of the present disclosure may help to downregulate survivin by complementary binding between a portion of an mRNA encoding survivin and a binding site of an engineered RNA. The binding site may be 70% complementary to a sequence found in an RNA encoding the survivin protein. In other instances, the binding site may exhibit at or over: 70 percent, 80 percent, 85 percent, 90 percent, 95 percent or more complementary binding between a binding site of an engineered nucleic acid and a target sequence of an RNA encoding the survivin protein. In some cases, the binding site may exhibit perfect complementary binding between the engineered nucleic acid and the target sequence. A DNA sequence corresponding to the survivin RNA may be found on GenBank with accession number NM_001012270.2 is shown below in Table 3 (SEQ. ID. NO 19). A binding site with a reverse compliment to a portion of the survivin RNA is found in Table 3 (SEQ ID NO: 20). It should be noted that the binding sites may not be exclusive to those listed.

### AKT2

AKT2 is an enzyme that in humans is encoded by the AKT2 gene. Further, it is a putative oncogene belonging to the AKT subfamily of serine/threonine kinases that contain SH2-like (Src homology 2-like) sites. The gene has been shown to be amplified and overexpressed in ovarian carcinoma cell lines and primary ovarian tumors. Overexpression additionally contributes to the malignant phenotype of a subset of human ductal pancreatic cancers.

An engineered RNA of the present disclosure may help to downregulate AKT2 protein by complementary binding between a portion of an mRNA encoding AKT2 and a binding site of an engineered RNA. The binding site may be 70% complementary to a sequence found in an RNA encoding the AKT2 protein. In other instances, the binding site may exhibit at or over: 70 percent, 80 percent, 85 percent, 90 percent, 95 percent or more complementary binding between a binding site of an engineered nucleic acid and a target sequence of an RNA encoding the AKT2 protein. In some cases, the binding site may exhibit perfect complementary binding between the engineered nucleic acid and the target sequence. A DNA sequence corresponding to the survivin RNA may be found on GenBank with accession number NM_001626.6 is shown below in Table 3 (SEQ. ID. NO 21). A binding site with a reverse compliment to a portion of the AKT2 RNA is found in Table 3 (SEQ ID NO: 22).

Potential target sequences or genes encoding target sequences for engineered nucleic acids disclosed herein are shown in Table 3. It should be noted that the binding sites may not be exclusive to those listed. Any DNA sequence provided herein also includes the RNA sequence wherein all T's are substituted for U's. Table 3 also provides reverse complement sequences that can be incorporated into a engineered nucleic acid design. In some cases, an engineered nucleic acid herein can comprise a sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 2, 4, 6, 8, 10, 12, 14, 16, 18, 20, or 22.

TABLE 3
Potential target sequences of viral mediated diseases and cancers

| Sequence | | |
|---|---|---|
| ID | Description | Sequence |
| SEQ ID | HIV-1 Tat | CTTTGAGCCAATTCCCATACATTATTGTGCCCCGGCTGGTTTTGCGATT |
| NO: 1 | GenBank accession | CTAAAATGTAATAATAAGACGTTCAATGGAACAGGACCATGTACAAAT |
| | number: NC_001802 | GTCAGCACAGTACAATGTACACA |
| SEQ ID | A 20 nt reverse | UUAUUACAUUUUAGAAUCGC |
| NO: 2 | compliment RNA of | |
| | Tat for engineered | |
| | binding site | |
| SEQ ID | Dengue virus 3 | AATGAAATGGGATTGTTGGAAACCACAAAGAGAGATTTGGGGATGTCC |
| NO: 3 | NS4B | AAAGAACCAGGTGTTGTTTCGCCAACCAGTTATTTGGATGTGGATTTAC |
| | GenBank Accession | ACCCAGCATCAGCCTGGACATTGTACGCTGTGGCCACAACAGTAATAA |
| | Number: JN406515 | CACCAATGTTGAGACATACCATAGAGAATTCCACAGCAAATGTGTCCC |
| | | TAGCAGCTATAGCCAACCAGGCAGTGGTCCTGATGGGTTTAGACAAAG |
| | | GATGGCCGATATCGAAAATGGACTTAGGCGTGCCACTATTGGCACTGG |
| | | GTTGCTATTCACAAGTGAACCCACTAACTCTCACAGCGGCAGTACTCCT |
| | | GCTAGTCACGCATTATGCTATTATAGGTCCAGGATTGCAGGCAAAAGC |
| | | TACTCGTGAGGCTCAAAAAAGGACGGCTGCTGGAATAATGAAGAATCC |
| | | AACGGTGGATGGGATAATGACAATAGACCTAGATCCTGTAATATACGA |
| | | CTCAAAATTTGAAAAACAACTAGGACAAGTTATGCTCCTGGTTCTGTGT |
| | | GCAGTTCAACTTTTGTTAATGAGAACATCATGGGCATTTTGTGAAGCTC |
| | | TAACCCTAGCCACAGGACCAATAACAACACTCTGGGAAGGATCACCTG |
| | | GGAAGTTCTGGAACACCACGATAGCTGTTTCCATGGCAAACATCTTTA |
| | | GAGGGAGCTATCTGGCAGGAGCTGGGCTAGCTTTTTCTATCATGAAAT |
| | | CAGTTGGAACAGGAAAGAGA |
| SEQ ID | A 20 nt reverse | AGUGCCAAUAGUGGCACGCC |
| NO: 4 | compliment RNA of | |
| | Dengue virus NS4B | |
| | for engineered | |
| | binding site | |
| SEQ ID | SARS-CoV-2 RNA | TCAGCTGATGCACAATCGTTTTTAAACACCGTGCGGCACAGGCACTAG |
| NO: 5 | dependent RNA | TACTGATGTCGTATACAGGGCTTTTGACATCTACAATGATAAAGTAGCT |
| | Polymerase DNA | GGTTTTGCTAAATTCCTAAAAACTAATTGTTGTCGCTTCCAAGAAAAGG |
| | sequence | ACGAAGATGACAATTTAATTGATTCTTACTTTGTAGTTAAGAGACACAC |
| | GenBank Accession | TTTCTCTAACTACCAACATGAAGAAACAATTTATAATTTACTTAAGGAT |
| | Number: | TGTCCAGCTGTTGCTAAACATGACTTCTTTAAGTTTAGAATAGACGGTG |
| | NC_045512.2 | ACATGGTACCACATATATCACGTCAACGTCTTACTAAATACACAATGG |
| | | CAGACCTCGTCTATGCTTTAAGGCATTTTGATGAAGGTAATTGTGACAC |
| | | ATTAAAAGAAATACTTGTCACATACAATTGTTGTGATGATGATTATTTC |
| | | AATAAAAAGGACTGGTATGATTTTGTAGAAAACCCAGATATATTACGC |
| | | GTATACGCCAACTTAGGTGAACGTGTACGCCAAGCTTTGTTAAAAACA |
| | | GTACAATTCTGTGATGCCATGCGAAATGCTGGTATTGTTGGTGTACTGA |
| | | CATTAGATAATCAAGATCTCAATGGTAACTGGTATGATTTCGGTGATTT |
| | | CATACAAACCACGCCAGGTAGTGGAGTTCCTGTTGTAGATTCTTATTAT |
| | | TCATTGTTAATGCCTATATTAACCTTGACCAGGGCTTTAACTGCAGAGT |
| | | CACATGTTGACACTGACTTAACAAAGCCTTACATTAAGTGGGATTTGTT |
| | | AAAATATGACTTCACGGAAGAGAGGTTAAAACTCTTTGACCGTTATTTT |
| | | AAATATTGGGATCAGACATACCACCCAAATTGTGTTAACTGTTTGGAT |
| | | GACAGATGCATTCTGCATTGTGCAAACTTTAATGTTTTATTCTCTACAG |
| | | TGTTCCCACCTACAAGTTTTGGACCACTAGTGAGAAAAATATTTGTTGA |
| | | TGGTGTTCCATTTGTAGTTTCAACTGGATACCACTTCAGAGAGCTAGGT |
| | | GTTGTACATAATCAGGATGTAAACTTACATAGCTCTAGACTTAGTTTTA |
| | | AGGAATTACTTGTGTATGCTGCTGACCCTGCTATGCACGCTGCTTCTGG |
| | | TAATCTATTACTAGATAAACGCACTACGTGCTTTTCAGTAGCTGCACTT |
| | | ACTAACAATGTTGCTTTTCAAACTGTCAAACCCGGTAATTTTAACAAAG |
| | | ACTTCTATGACTTTGCTGTGTCTAAGGGTTTCTTTAAGGAAGGAAGTTC |
| | | TGTTGAATTAAAACACTTCTTCTTTGCTCAGGATGGTAATGCTGCTATC |
| | | AGCGATTATGACTACTATCGTTATAATCTACCAACAATGTGTGATATCA |
| | | GACAACTACTATTTGTAGTTGAAGTTGTTGATAAGTACTTTGATTGTTA |
| | | CGATGGTGGCTGTATTAATGCTAACCAAGTCATCGTCAACAACCTAGA |
| | | CAAATCAGCTGGTTTTCCATTTAATAAATGGGGTAAGGCTAGACTTTAT |
| | | TATGATTCAATGAGTTATGAGGATCAAGATGCACTTTTCGCATATACAA |
| | | AACGTAATGTCATCCCTACTATAACTCAAATGAATCTTAAGTATGCCAT |
| | | TAGTGCAAAGAATAGAGCTCGCACCGTAGCTGGTGTCTCTATCTGTAG |
| | | TACTATGACCAATAGACAGTTTCATCAAAAATTATTGAAATCAATAGC |
| | | CGCCACTAGAGGAGCTACTGTAGTAATTGGAACAAGCAAATTCTATGG |
| | | TGGTTGGCACAACATGTTAAAAACTGTTTATAGTGATGTAGAAAACCC |
| | | TCACCTTATGGGTTGGGATTATCCTAAATGTGATAGAGCCATGCCTAAC |
| | | ATGCTTAGAATTATGGCCTCACTTGTTCTTGCTCGCAAACATACAACGT |
| | | GTTGTAGCTTGTCACACCGTTTCTATAGATTAGCTAATGAGTGTGCTCA |
| | | AGTATTGAGTGAAATGGTCATGTGTGGCGGTTCACTATATGTTAAACC |
| | | AGGTGGAACCTCATCAGGAGATGCCACAACTGCTTATGCTAATAGTGT |
| | | TTTTAACATTTGTCAAGCTGTCACGGCCAATGTTAATGCACTTTTATCT |
| | | ACTGATGGTAACAAAATTGCCGATAAGTATGTCCGCAATTTACAACAC |
| | | AGACTTTATGAGTGTCTCTATAGAAATAGAGATGTTGACACAGACTTT |
| | | GTGAATGAGTTTTACGCATATTTGCGTAAACATTTCTCAATGATGATAC |
| | | TCTCTGACGATGCTGTTGTGTGTTTCAATAGCACTTATGCATCTCAAGG |
| | | TCTAGTGGCTAGCATAAAGAACTTTAAGTCAGTTCTTTATTATCAAAAC |
| | | AATGTTTTTATGTCTGAAGCAAAATGTTGGACTGAGACTGACCTTACTA |
| | | AAGGACCTCATGAATTTTGCTCTCAACATACAATGCTAGTTAAACAGG |
| | | GTGATGATTATGTGTACCTTCCTTACCCAGATCCATCAAGAATCCTAGG |
| | | GGCCGGCTGTTTTGTAGATGATATCGTAAAAACAGATGGTACACTTAT |
| | | GATTGAACGGTTCGTGTCTTTAGCTATAGATGCTTACCCACTTACTAAA |
| | | CATCCTAATCAGGAGTATGCTGATGTCTTTCATTTGTACTTACAATACA |
| | | TAAGAAAGCTACATGATGAGTTAACAGGACACATGTTAGACATGTATT |
| | | CTGTTATGCTTACTAATGATAACACTTCAAGGTATTGGGAACCTGAGTT |
| | | TTATGAGGCTATGTACACACCGCATACAGTCTTACAG |
| SEQ ID | A 20 nt reverse | CUAGCCACUAGACCUUGAGA |
| NO: 6 | compliment RNA of | |
| | SARS-CoV-2 DNA | |
| | sequence | |
| | Polymerase for | |
| | engineered binding | |
| | site | |
| SEQ ID | HHV4 (Epstein Barr | ATGGAAACCACTCAGACTCTCCGCTTTAAGACCAAGGCCCTAGCCGTC |
| NO: 7 | virus) | CTGTCCAAGTGCTATGACCATGCCCAGACTCATCTCAAGGGAGGAGTG |
| | HHV4 BMRF1 | CTGCAGGTAAACCTTCTGTCTGTAAACTATGGAGGCCCCCGGCTGGCC |
| | DNA polymerase | GCCGTGGCCAACGCAGGCACGGCCGGGCTAATCAGCTTCGAGGTCTCC |
| | processivity subunit | CCTGACGCTGTGGCCGAGTGGCAGAATCACCAGAGCCCAGAGGAGGC |
| | GenBank Accession | CCCGGCCGCCGTGTCATTTAGAAACCTTGCCTACGGGCGCACCTGTGTC |
| | Number: | CTGGGCAAGGAGCTGTTTGGCTCGGCTGTGGAGCAGGCTTCCCTGCAA |
| | NC_007605.1 | TTTTACAAGCGGCCACAAGGGGGTTCCCGGCCTGAATTTGTTAAGCTC |
| | | ACTATGGAATATGATGATAAGGTGTCCAAGAGCCACCACACCTGCGCC |
| | | CTGATGCCCTATATGCCCCCGGCCAGCGACAGGCTGAGGAACGAGCAG |
| | | ATGATTGGGCAGGTGCTGTTGATGCCCAAGACGGCTTCCTCGTTGCAG |
| | | AAGTGGGCACGCCAGCAAGGCTCAGGCGGCGTTAAGGTGACACTCAAT |
| | | CCGGATCTCTACGTCACCACGTATACTTCTGGGGAGGCCTGCCTCACCC |
| | | TAGACTACAAGCCTCTGAGTGTGGGGCCATACGAGGCCTTCACTGGCC |
| | | CTGTGGCCAAGGCTCAGGACGTGGGGGCCGTTGAGGCCCACGTTGTCT |
| | | GCTCGGTAGCAGCGGACTCGCTGGCGGCGGCGCTTAGCCTCTGCCGCA |
| | | TTCCGGCCGTTAGCGTGCCAATCTTGAGGTTTTACAGGTCTGGCATCAT |
| | | AGCTGTGGTGGCCGGCCTGCTGACGTCAGCGGGGGACCTGCCGTTGGA |
| | | TCTTAGTGTTATTTTATTTAACCACGCCTCCGAAGAGGCGGCCGCCAGT |
| | | ACGGCCTCTGAGCCAGAAGATAAAAGTCCCCGGGTGCAACCACTGGGC |
| | | ACAGGACTCCAACAACGCCCCAGACATACGGTCAGTCCATCTCCTTCA |
| | | CCTCCGCCACCTCCTAGGACCCCTACTTGGGAGAGTCCGGCAAGGCCA |
| | | GAGACACCCTCGCCTGCCATTCCCAGCCACTCCAGCAACACCGCACTG |
| | | GAGAGGCCTCTGGCTGTTCAGCTCGCGAGGAAAAGGACATCGTCGGAG |
| | | GCCAGGCAGAAGCAGAAGCACCCCAAGAAAGTGAAGCAGGCCTTTAA |
| | | CCCCCTCATTTAA |
| SEQ ID | A 20 nt reverse | CAGUGAAGGCCUCGUAUGGC |
| NO: 8 | compliment RNA of | |
| | HHV4 BMRF1 | |
| | DNA polymerase | |
| | processivity subunit | |
| | DNA sequence | |
| | for engineered | |
| | binding site | |
| SEQ ID | Influenza H1N1 | GGAGCAAAAGCAGGTAGATATTGAAAGATGAGTCTTCTAACCGAGGTC |
| NO: 9 | strain Matrix | GAAACGTACGTTCTCTCTATCATCCCGTCAGGCCCCCTCAAAGCCGAG |
| | protein 1 | ATCGCACAGAGACTTGAAGATGTATTTGCTGGAAAGAATACCGATCTT |
| | GenBank Accession | GAGGCTCTCATGGAGTGGCTAAAGACAAGACCAATCCTGTCACCTCTG |
| | Number: | ACTAAGGGGATTTTAGGATTTGTGTTCACGCTCACCGTGCCCAGTGAGC |
| | CY043484.1 | GAGGACTGCAGCGTAGACGCTTTGTCCAAAATGCCCTCAATGGGAATG |
| | | GGGATCCAAATAATATGGACAGAGCAGTCAAACTTTATCGAAAGCTTA |
| | | AGAGGGAGATAACATTCCATGGGGCCAAAGAAATAGCACTCAGTTATT |
| | | CTGCTGGTGCACTTGCCAGTTGTATGGGACTCATATACAACAGGATGG |
| | | GGGCTGTGACCACCGAATCAGCATTTGGCCTTATATGTGCAACCTGTG |
| | | AACAGATTGCCGACTCCCAGCATAAGTCTCACAGGCAAATGGTAACAA |
| | | CAACCAATCCATTAATAAGACATGAGAACAGAATGGTTCTGGCCAGCA |
| | | CTACAGCTAAGGCTATGGAGCAAATGGCTGGATCGAGCGAACAAGCA |
| | | GCTGAGGCCATGGAGGTTGCTAGTCAGGCCAGGCAGATGGTGCAGGCA |
| | | ATGAGAGCCATTGGGACTCATCCTAGCTCTAGCACTGGTCTGAAAAAT |
| | | GATCTCCTTGAAAATTTACAGGCCTATCAGAAACGAATGGGGGTGCAG |
| | | ATGCAACGATTCAAGTGATCCTCTTGTTGTTGCCGCAAGTATAATTGGG |
| | | ATTGTGCACTTGATATTGTGGATTATTGATCGCCTTTTTTCCAAAAGCA |
| | | TTTATCGTATCTTTAAACACGGTTTAAAAAGAGGGCCTTCTACGGAAG |
| | | GAGTACCAGAGTCTATGAGGGAAGAATATCGAGAGGAACAGCAGAAT |
| | | GCTGTGGATGCTGACGATGATCATTTTGTCAGCATAGAGCTAGAGTAA |
| | | AAAACTC |
| SEQ ID | A 20 nt reverse | UGCAGUCCUCGCUCACUGGG |
| NO: 10 | compliment RNA of | |
| | Influenza H1N1 | |
| | strain Matrix | |
| | protein 1 DNA | |
| | sequence for | |
| | engineered binding | |
| | site | |
| SEQ ID | Rabies RNA | ATGCTCGATCCTGGAGAGGTCTATGATGACCCTATTGACCCAATCGAG |
| NO: 11 | dependent RNA | TTAGAGGCTGAACCCAGAGGAACCCCCATTGTCCCCAACATCTTGAGG |
| | polymerase | AACTCTGACTACAATCTCAACTCTCCTTTGATAGAAGATCCTGCTAGAC |
| | GenBank Accession | TAATGTTAGAATGGTTAAAAACAGGGAATAGACCTTATCGGATGACTC |
| | Number: M31046.1 | TAACAGACAATTGCTCCAGGTCTTTCAGAGTTTTGAAAGATTATTTCAA |
| | | GAAGGTAGATTTGGGTTCTCTCAAGGTGGGCGGAATGGCTGCACAGTC |
| | | AATGATTTCTCTCTGGTTATATGGTGCCCACTCTGAATCCAACAGGAGC |
| | | CGGAGATGTATAACAGACTTGGCCCATTTCTATTCCAAGTCGTCCCCCA |
| | | TAGAGAAGCTGTTGAATCTCACGCTAGGAAATAGAGGGCTGAGAATCC |
| | | CCCCAGAGGGAGTGTTAAGTTGCCTTGAGAGGGTTGATTATGATAATG |
| | | CATTTGGAAGGTATCTTGCCAACACGTATTCCTCTTACTTGTTCTTCCAT |
| | | GTAATCACCTTATACATGAACGCCCTAGACTGGGATGAAGAAAAGACC |
| | | ATCCTAGCATTATGGAAAGATTTAACCTCAGTGGACATCGGGAAGGAC |
| | | TTGGTAAAGTTCAAAGACCAAATATGGGGACTGCTGATCGTGACAAAG |
| | | GACTTTGTTTACTCCCAAAGTTCCAATTGTCTTTTTGACAGAAACTACA |
| | | CACTTATGCTAAAAGATCTTTTCTTGTCTCGCTTCAACTCCTTAATGGTC |
| | | TTGCTCTCTCCCCCAGAGCCCCGATACTCAGATGACTTGATATCTCAAC |
| | | TATGCCAGCTGTACATTGCTGGGGATCAAGTCTTGTCTATGTGTGGAAA |
| | | CTCCGGCTATGAAGTCATCAAAATATTGGAGCCATATGTCGTGAATAG |
| | | TTTAGTCCAGAGAGCAGAAAAGTTTAGGCCTCTCATTCATTCCTTGGGA |
| | | GACTTTCCTGTATTTATAAAAGACAAGGTAAGTCAACTTGAAGAGACG |
| | | TTCGGTCCCTGTGCAAGAAGGTTCTTTAGGGCTCTGGATCAATTCGACA |
| | | ACATACATGACTTGGTTTTTGTGTTTGGCTGTTACAGGCATTGGGGGCA |
| | | CCCATATATAGATTATCGAAAGGGTCTGTCAAAACTATATGATCAGGT |
| | | TCACCTTAAAAAAATGATAGATAAGTCCTACCAGGAGTGCTTAGCAAG |
| | | CGACCTAGCCAGGAGGATCCTTAGATGGGGTTTTGATAAGTACTCCAA |
| | | GTGGTATCTGGATTCAAGATTCCTAGCCCGAGACCACCCCTTGACTCCT |
| | | TATATCAAAACCCAAACATGGCCACCCAAACATATTGTAGACTTGGTG |
| | | GGGGATACATGGCACAAGCTCCCGATCACGCAGATCTTTGAGATTCCT |
| | | GAATCAATGGATCCGTCAGAAATATTGGATGACAAATCACATTCTTTC |
| | | ACCAGAACGAGACTAGCTTCTTGGCTGTCAGAAAACCGAGGGGGGCCT |
| | | GTTCCTAGCGAAAAAGTTATTATCACGGCCCTGTCTAAGCCGCCTGTCA |
| | | ATCCCCGAGAGTTTCTGAGGTCTATAGACCTCGGAGGATTGCCAGATG |
| | | AAGACTTGATAATTGGCCTCAAGCCAAAGGAACGGGAATTGAAGATTG |
| | | AAGGTCGATTCTTTGCTCTAATGTCATGGAATCTAAGATTGTATTTTGT |
| | | CATCACTGAAAAACTCTTGGCCAACTACATCTTGCCACTTTTTGACGCG |
| | | CTGACTATGACAGACAACCTGAACAAGGTGTTTAAAAAGCTGATCGAC |
| | | AGGGTCACCGGGCAAGGGCTTTTGGACTATTCAAGGGTCACATATGCA |
| | | TTTCACCTGGACTATGAAAAGTGGAACAACCATCAAAGATTAGAGTCA |
| | | ACAGAGGATGTATTTTCTGTCCTAGATCAAGTGTTTGGATTGAAGAGA |
| | | GTGTTTTCTAGAACACACGAGTTTTTTCAAAAGGCCTGGATCTATTATT |
| | | CAGACAGATCAGACCTCATCGGGTTACGGGAGGATCAAATATACTGCT |
| | | TAGATGCGTCCAACGGCCCAACCTGTTGGAATGGCCAGGATGGCGGGC |
| | | TAGAAGGCTTACGGCAGAAGGGCTGGAGTCTAGTCAGCTTATTGATGA |
| | | TAGATAGAGAATCTCAAATCAGGAACACAAGAACCAAAATACTAGCTC |
| | | AAGGAGACAACCAGGTTTTATGTCCGACATACATGTTGTCGCCAGGGC |
| | | TATCTCAAGAGGGGCTCCTCTATGAATTGGAGAGAATATCAAGGAATG |
| | | CACTTTCGATATACAGAGCCGTCGAGGAAGGGGCATCTAAGCTAGGGC |
| | | TGATCATCAAGAAAGAAGAGACCATGTGTAGTTATGACTTCCTCATCT |
| | | ATGGAAAAACCCCTTTGTTTAGAGGTAACATATTGGTGCCTGAGTCCA |
| | | AAAGATGGGCCAGAGTCTCTTGCGTCTCTAATGACCAAATAGTCAACC |
| | | TCGCCAATATAATGTCGACAGTGTCCACCAATGCGCTAACAGTGGCAC |
| | | AACACTCTCAATCTTTGATCAAACCGATGAGGGATTTTCTGCTCATGTC |
| | | AGTACAGGCAGTCTTTCACTACCTGCTATTTAGCCCAATCTTAAAGGGA |
| | | AGAGTTTACAAGATTCTGAGCGCTGAAGGGGAGAGCTTTCTCCTAGCC |
| | | ATGTCAAGGATAATCTATCTAGATCCTTCTTTGGGAGGGATATCTGGAA |
| | | TGTCCCTCGGAAGATTCCATATACGACAGTTCTCAGACCCTGTCTCTGA |
| | | AGGGTTATCCTTCTGGAGAGAGATCTGGTTAAGCTCCCAAGAGTCCTG |
| | | GATTCACGCGTTGTGTCAAGAGGCTGGAAACCCAGATCTTGGAGAGAG |
| | | AACACTCGAGAGCTTCACTCGCCTTCTAGAAGATCCGACCACCTTAAA |
| | | TATCAGAGGAGGGGCCAGTCCTACCATTCTACTCAAGGATGCAATCAG |
| | | AAAGGCTTTATATGACGAGGTGGACAAGGTGGAAAATTCAGAGTTTCG |
| | | AGAGGCAATCCTGTTGTCCAAGACCCATAGAGATAATTTTATACTCTTC |
| | | TTAATATCTGTTGAGCCTCTGTTTCCTCGATTTCTCAGTGAGCTATTCAG |
| | | TTCGTCTTTTTTGGGAATCCCCGAGTCAATCATTGGATTGATACAAAAC |
| | | TCCCGAACGATAAGAAGGCAGTTTAGAAAGAGTCTCTCAAAAACTTTA |
| | | GAAGAATCCTTCTACAACTCAGAGATCCACGGGATTAGTCGGATGACC |
| | | CAGACACCTCAGAGGGTTGGGGGGGTGTGGCCTTGCTCTTCAGAGAGG |
| | | GCAGATCTACTTAGGGAGATCTCTTGGGGAAGAAAAGTGGTAGGCACG |
| | | ACAGTTCCTCACCCTTCTGAGATGTTGGGATTACTTCCCAAGTCCTCTA |
| | | TTTCTTGCACTTGTGGAGCAACAGGAGGAGGCAATCCTAGAGTTTCTGT |
| | | ATCAGTACTCCCGTCCTTTGATCAGTCATTTTTTTCACGAGGCCCCCTA |
| | | AAGGGATACTTGGGCTCGTCCACCTCTATGTCGACCCAGCTATTCCATG |
| | | CATGGGAAAAAGTCACTAATGTTCATGTGGTGAAGAGAGCTCTATCGT |
| | | TAAAAGAATCTATAAACTGGTTCATTACTAGAGATTCCAACTTGGCTCA |
| | | AGCTCTAATTAGGAACATTATGTCTCTGACAGGCCCTGATTTCCCTCTA |
| | | GAGGAGGCCCCTGTCTTCAAAAGGACGGGGTCAGCCTTGCATAGGTTC |
| | | AAGTCTGCCAGATACAGCGAAGGAGGGTATTCTTCTGTCTGCCCGAAC |
| | | CTCCTCTCTCATATTTCTGTTAGTACAGACACCATGTCTGATTTGACCC |
| | | AAGACGGGAAGAACTACGATTTCATGTTCCAGCCATTGATGCTTTATG |
| | | CACAGACATGGACATCAGAGCTGGTACAGAGAGACACAAGGCTAAGA |
| | | GACTCTACGTTTCATTGGCACCTCCGATGCAACAGGTGTGTGAGACCC |
| | | ATTGACGACGTGACCCTGGAGACCTCTCAGATCTTCGAGTTTCCGGATG |
| | | TGTCGAAAAGAATATCCAGAATGGTTTCTGGGGCTGTGCCTCACTTCCA |
| | | GAGGCTTCCCGATATCCGTCTGAGACCAGGAGATTTTGAATCTCTAAG |
| | | CGGTAGAGAAAAGTCTCACCATATCGGATCAGCTCAGGGGCTCTTATA |
| | | CTCAATCTTAGTGGCAATTCACGACTCAGGATACAATGATGGAACCAT |
| | | CTTCCCTGTCAACATATACGGCAAGGTTTCCCCTAGAGACTATTTGAGA |
| | | GGGCTCGCAAGGGGAGTATTGATAGGATCCTCGATTTGCTTCTTGACA |
| | | AGAATGACAAATATCAATATTAATAGACCTCTTGAATTGGTCTCAGGG |
| | | GTAATCTCATATATTCTCCTGAGGCTAGATAACCATCCCTCCTTGTACA |
| | | TAATGCTCAGAGAACCGTCTCTTAGAGGAGAGATATTTTCTATCCCTCA |
| | | GAAAATCCCCGCCGCTTATCCAACCACTATGAAAGAAGGCAACAGATC |
| | | AATCTTGTGTTATCTCCAACATGTGCTACGCTATGAGCGAGAGATAATC |
| | | ACGGCGTCTCCAGAGAATGACTGGCTATGGATCTTTTCAGACTTTAGA |
| | | AGTGCCAAAATGACGTACCTATCCCTCATTACTTACCAGTCTCATCTTC |
| | | TACTCCAGAGGGTTGAGAGAAACCTATCTAAGAGTATGAGAGATAACC |
| | | TGCGACAATTGAGTTCTTTGATGAGGCAGGTGCTGGGCGGGCACGGAG |
| | | AAGATACCTTAGAGTCAGACGACAACATTCAACGACTGCTAAAAGACT |
| | | CTTTACGAAGGACAAGATGGGTGGATCAAGAGGTGCGCCATGCAGCTA |
| | | GAACCATGACTGGAGATTACAGCCCCAACAAGAAGGTGTCCCGTAAGG |
| | | TAGGATGTTCAGAATGGGTCTGCTCTGCTCAACAGGTTGCAGTCTCTAC |
| | | CTCAGCAAACCCGGCCCCTGTCTCGGAGCTTGACATAAGGGCCCTCTCT |
| | | AAGAGGTTCCAGAACCCTTTGATCTCGGGCTTGAGAGTGGTTCAGTGG |
| | | GCAACCGGTGCTCATTATAAGCTTAAGCCTATTCTAGATGATCTCAATG |
| | | TTTTCCCATCTCTCTGCCTTGTAGTTGGGGACGGGTCAGGGGGGATATC |
| | | AAGGGCAGTCCTCAACATGTTTCCAGATGCCAAGCTTGTGTTCAACAG |
| | | TCTTTTAGAGGTGAATGACCTGATGGCTTCCGGAACACATCCACTGCCT |
| | | CCTTCAGCAATCATGAGGGGAGGAAATGATATCGTCTCCAGAGTGATA |
| | | GATCTTGACTCAATCTGGGAAAAACCGTCCGACTTGAGAAACTTGGCA |
| | | ACCTGGAAATACTTCCAGTCAGTCCAAAAGCAGGTCAACATGTCCTAT |
| | | GACCTCATTATTTGCGATGCAGAAGTTACTGACATTGCATCTATCAACC |
| | | GGATCACCCTGTTAATGTCCGATTTTGCATTGTCTATAGATGGACCACT |
| | | CTATTTGGTCTTCAAAACTTATGGGACTATGCTAGTAAATCCAAACTAC |
| | | AAGGCTATTCAACACCTGTCAAGAGCGTTCCCCTCGGTCACAGGGTTT |
| | | ATCACCCAAGTAACTTCGTCTTTTTCATCTGAGCTCTACCTCCGATTCTC |
| | | CAAACGAGGGAAGTTTTTCAGAGATGCTGAGTACTTGACCTCTTCCAC |
| | | CCTTCGAGAAATGAGCCTTGTGTTATTCAATTGTAGCAGCCCCAAGAGT |
| | | GAGATGCAGAGAGCTCGTTCCTTGAACTATCAGGATCTTGTGAGAGGA |
| | | TTTCCTGAAGAAATCATATCAAATCCTTACAATGAGATGATCATAACTC |
| | | TGATTGACAGTGATGTAGAATCTTTTCTAGTCCACAAGATGGTTGATGA |
| | | TCTTGAGTTACAGAGGGGAACTCTGTCTAAAGTGGCTATCATTATAGCC |
| | | ATCATGATAGTTTTCTCCAACAGAGTCTTCAACGTTTCCAAACCCCTAA |
| | | CTGACCCCTCGTTCTATCCACCGTCTGATCCCAAAATCCTGAGGCACTT |
| | | CAACATATGTTGCAGTACTATGATGTATCTATCTACTGCTTTAGGTGAC |
| | | GTCCCTAGCTTCGCAAGACTTCACGACCTGTATAACAGACCTATAACTT |
| | | ATTACTTCAGAAAGCAAGTCATTCGAGGGAACGTTTATCTATCTTGGA |
| | | GTTGGTCCAACGACACCTCAGTGTTCAAAAGGGTAGCCTGTAATTCTA |
| | | GCCTGAGTCTGTCATCTCACTGGATCAGGTTGATTTACAAGATAGTGAA |
| | | GACTACCAGACTCGTTGGCAGCATCAAGGATCTATCCAGAGAAGTGGA |
| | | AAGACACCTTCATAGGTACAACAGGTGGATCACCCTAGAGGATATCAG |
| | | ATCTAGATCATCCCTACTAGACTACAGTTGCCTGTGA |

| | | |
|---|---|---|
| SEQ ID | A 20 nt reverse | UAUAAUGAGCACCGGUUGCC |
| NO: 12 | compliment RNA of | |
| | Rabies RNA | |
| | dependent RNA | |
| | polymerase DNA | |
| | sequence | |
| | for engineered | |
| | binding site | |
| SEQ ID | Smallpox EV | TTATAATTTTACCATTTGACTCATGACTTCATTGATATCTTTATACGAGC |
| NO: 13 | protein | TACGAATATATAATTCTTTATAACTGAACTGAGATATATACACTGGATC |
| | GenBank Accession | TATGGTTTCCATAATTGAGTAAATAAATGCTCTGCAATAACTAATGGCA |
| | Number: | AATGTATAAAACAACAAAATTATACTAGAGTTGTTAAAGTTAATATTTT |
| | NC_001611.1 | CTATTAGTTGTTCCAATAAATTATTTGTTGTGACTGTGTTCAAGTCATA |
| | | AATCATTTTGATACTATCCAATAAACAGTCTTTAAGTTCTGGAATATTA |
| | | TCATCCCATTGTAAAGCCCCTAATTCGACTATCGAATACCCTGCTCTGG |
| | | TAGCCGTTTCAATATCGACGGACGTCAATACTGTAATAAAGGTGGTAG |
| | | TATTGTCATCATCATGATACACAACAGGAATATGGTCGTTAGTAGGTA |
| | | CGGTGACTTTACACAACGCGATATATAACTTTCCTTTTGTGCCATTTTT |
| | | AACGTAGTTGGGACGTCCTGCAGGGTATTGTTTTGAAGAAATGATATC |
| | | GAGAACAGATTTGATACGATATTTGTTGGATTTCTGATTATTCACTATA |
| | | ATATAATCTAGACAGATAGATGATTCGATAAATAAAGAAGATATATCG |
| | | TTGGTAGGATAATACATCCCCATTCCAGTATTCTTGGATACTCTATTGA |
| | | TGACACTAGTTAAGAACATGTCTTCAATTCTAGAAAACGAAAACATCC |
| | | TACACGGACTCATTAAAACTTCTAGCGCTCCTGATTGTGTCTCAAATGC |
| | | CTCATACAATGATTTCAAGGATGTCATAGATTCTTTAACCCACGATTTA |
| | | ATATTGCGTTTAGCATCTATTTTTTTTATTAAATCGAATGGTCGGCTCTC |
| | | TGGTTTGCTACCCCAATGATAACAATAGTCTTGTAAAGATAAACCGCA |
| | | AGAAAATTTATACGCATCCATCCAAATAACTCTTGCACCATCAGATGA |
| | | TATCAGTATATTATTATAGATTTTCCATCCACAATTATTGGGCCAGTAT |
| | | ACTGTTAGCAACGGTATATCGAATAGATTACTCATGTAACCTACTAGA |
| | | ATGATAGTTCGTGTACTAGTCATAATATCTTTAATCCAATCTAAGAAAT |
| | | TTAAAATTAGATCTTTTACACTGTTAAAGTTAACAAAGGTATTACCCGG |
| | | GTACGTGGATATCATATATGGCATTGGTCCATTATCAGTAATAGCTCCA |
| | | TAAACTGATACGGTGATGGTTTTTATATGTGTTTGATCTAACGAGGAAG |
| | | AAATTTGCGCCCATAATTCATCTCTAGATATGTATTTAATATCAAACGG |
| | | TAACACATCAATTTCGGGACGCGTATATGTTTCTAAATTTTTAATCCAA |
| | | ATATAATGATGACCTATATGCCCTATTATCATACTGTCAACTATAGTAC |
| | | ACTTAGAGAACTTACGATACATCTGTTTCCTATAATCGTTAAATTTTAC |
| | | AAATCTATAACATGCTAAACCTTTTGACGACAACCATTCATTAATTTCT |
| | | GATATGGAATCTGTATTCTCGATACAGTATCGTTTTAAAGCCAGTGCTA |
| | | TATCTCCCTGTTCGTGAGAACGCTTTCGTATAATATCAATCAACGGATA |
| | | ATCTGAGGTTTTTGGAGAATAATATGACTCATGATCTATTTCGTCCATA |
| | | AACAATCTAGACATAGGAATTGGAGGCGATGATCTTAATTTTGTGCAA |
| | | TGAGTAGTTAATCCTATAACTTCTAATCTTGTAATATTCATCATCGACA |
| | | TAATACTATCTATGTTATCATCGTATATTAGTATACCATGACCTTCTTCA |
| | | TTTCGTGCCAAAATGATATACAGTCTTAAATAGTTACGCAATATCTCAA |
| | | TAGTTTCATAATTGTTAGCTGTTTTCATTAAGATTTGTACCCTGTTTAAC |
| | | AT |
| SEQ ID | A 20 nt reverse | CAGGGAGAUAUAGCACUGGC |
| NO: 14 | compliment RNA of | |
| | Smallpox EV | |
| | protein DNA | |
| | sequence | |
| | for engineered | |
| | binding site | |
| SEQ ID | cDNA CCL2 protein | GAGAGGCTGAGACTAACCCAGAAACATCCAATTCTCAAACTGAAGCTC |
| NO: 15 | GenBank Accession | GCACTCTCGCCTCCAGCATGAAAGTCTCTGCCGCCCTTCTGTGCCTGCT |
| | Number: | GCTCATAGCAGCCACCTTCATTCCCCAAGGGCTCGCTCAGCCAGATGC |
| | AK311960.1 | AATCAATGCCCCAGTCACCTGCTGCTATAACTTCACCAATAGGAAGAT |
| | | CTCAGTGCAGAGGCTCGCGAGCTATAGAAGAATCACCAGCAGCAAGTG |
| | | TCCCAAAGAAGCTGTGATCTTCAAGACCATTGTGGCCAAGGAGATCTG |
| | | TGCTGACCCCAAGCAGAAGTGGGTTCAGGATTCCATGGACCACCTGGA |
| | | CAAGCAAACCCAAACTCCGAAGACTTGA |
| SEQ ID | A 20 nt reverse | UAUAGCAGCAGGUGACUGGG |
| NO: 16 | compliment RNA of | |
| | CCL2 | |
| | for engineered | |
| | binding site | |
| SEQ ID | N-myc cDNA | AGGCTGTGACAGTCATCTGTCTGGACGCGCTGGGTGGATGCGGGGGGC |
| NO: 17 | GenBank Accession | TCCTGGGAACTGTGTTGGAGCCGAGCAAGCGCTAGCCAGGCGCAAGCG |
| | Number: | CGCACAGACTGTAGCCATCCGAGGACACCCCCGCCCCCCCGGCCCACC |
| | NM_001293228.2 | CGGAGACACCCGCGCAGAATCGCCTCCGGATCCCCTGCAGTCGGCGGG |
| | | AGGTAAGGAGCAGGGCTTGCAAACCGCCCGGCGCCCAGGGAAGCGAC |
| | | GAGCGCCGGGGCAAGGCAAGCCCTGGACGGGATTGCGACGTGCGCAC |
| | | CGGGCGCCCTAATATGCCCGGGGGACTGTTTCTGCTTCCGAAACAAAA |
| | | CCATCTCTGGGTTTTCCCAGAAAAGCCAGTTCCAGCCCCGAAGGCATC |
| | | CTGGCTAGAGGAGACCCGCCCTAATCCTTTTGCAGCCCTTACCGGGGG |
| | | GAGTAATGGCTTCTGCGAAAAGAAATTCCCTCGGCTCTAGAAGATCTG |
| | | TCTGTGTTTGAGCTGTCGGAGAGCCGTGTTGGAGGTCGGCGCCGGCCC |
| | | CCGCCTTCCGCGCCCCCCACGGGAAGGAAGCACCCCCGGTATTAAAAC |
| | | GAACGGGGCGGAAAGAAGCCCTCAGTCGCCGGCCGGGAGGCGAGCCG |
| | | ATGCCGAGCTGCTCCACGTCCACCATGCCGGGCATGATCTGCAAGAAC |
| | | CCAGACCTCGAGTTTGACTCGCTACAGCCCTGCTTCTACCCGGACGAA |
| | | GATGACTTCTACTTCGGCGGCCCCGACTCGACCCCCCCGGGGGAGGAC |
| | | ATCTGGAAGAAGTTTGAGCTGCTGCCCACGCCCCCGCTGTCGCCCAGC |
| | | CGTGGCTTCGCGGAGCACAGCTCCGAGCCCCCGAGCTGGGTCACGGAG |
| | | ATGCTGCTTGAGAACGAGCTGTGGGGCAGCCCGGCCGAGGAGGACGC |
| | | GTTCGGCCTGGGGGGACTGGGTGGCCTCACCCCCAACCCGGTCATCCT |
| | | CCAGGACTGCATGTGGAGCGGCTTCTCCGCCCGCGAGAAGCTGGAGCG |
| | | CGCCGTGAGCGAGAAGCTGCAGCACGGCCGCGGGCCGCCAACCGCCG |
| | | GTTCCACCGCCCAGTCCCCGGGAGCCGGCGCCGCCAGCCCTGCGGGTC |
| | | GCGGGCACGGCGGGGCTGCGGGAGCCGGCCGCGCCGGGGCCGCCCTG |
| | | CCCGCCGAGCTCGCCCACCCGGCCGCCGAGTGCGTGGATCCCGCCGTG |
| | | GTCTTCCCCTTTCCCGTGAACAAGCGCGAGCCAGCGCCCGTGCCCGCA |
| | | GCCCCGGCCAGTGCCCCGGCGGCGGGCCCTGCGGTCGCCTCGGGGGCG |
| | | GGTATTGCCGCCCCAGCCGGGGCCCCGGGGGTCGCCCCTCCGCGCCCA |
| | | GGCGGCCGCCAGACCAGCGGCGGCGACCACAAGGCCCTCAGTACCTCC |
| | | GGAGAGGACACCCTGAGCGATTCAGATGATGAAGATGATGAAGAGGA |
| | | AGATGAAGAGGAAGAAATCGACGTGGTCACTGTGGAGAAGCGGCGTT |
| | | CCTCCTCCAACACCAAGGCTGTCACCACATTCACCATCACTGTGCGTCC |
| | | CAAGAACGCAGCCCTGGGTCCCGGGAGGGCTCAGTCCAGCGAGCTGAT |
| | | CCTCAAACGATGCCTTCCCATCCACCAGCAGCACAACTATGCCGCCCC |
| | | CTCTCCCTACGTGGAGAGTGAGGATGCACCCCCACAGAAGAAGATAAA |
| | | GAGCGAGGCGTCCCCACGTCCGCTCAAGAGTGTCATCCCCCCAAAGGC |
| | | TAAGAGCTTGAGCCCCCGAAACTCTGACTCGGAGGACAGTGAGCGTCG |
| | | CAGAAACCACAACATCCTGGAGCGCCAGCGCCGCAACGACCTTCGGTC |
| | | CAGCTTTCTCACGCTCAGGGACCACGTGCCGGAGTTGGTAAAGAATGA |
| | | GAAGGCCGCCAAGGTGGTCATTTTGAAAAAGGCCACTGAGTATGTCCA |
| | | CTCCCTCCAGGCCGAGGAGCACCAGCTTTTGCTGGAAAAGGAAAAATT |
| | | GCAGGCAAGACAGCAGCAGTTGCTAAAGAAAATTGAACACGCTCGGA |
| | | CTTGCTAGACGCTTCTCAAAACTGGACAGTCACTGCCACTTTGCACATT |
| | | TTGATTTTTTTTTTAAACAAACATTGTGTTGACATTAAGAATGTTGGTTT |
| | | ACTTTCAAATCGGTCCCCTGTCGAGTTCGGCTCTGGGTGGGCAGTAGG |
| | | ACCACCAGTGTGGGGTTCTGCTGGGACCTTGGAGAGCCTGCATCCCAG |
| | | GATGCTGGGTGGCCCTGCAGCCTCCTCCACCTCACCTCCATGACAGCG |
| | | CTAAACGTTGGTGACGGTTGGGAGCCTCTGGGGCTGTTGAAGTCACCT |
| | | TGTGTGTTCCAAGTTTCCAAACAACAGAAAGTCATTCCTTCTTTTTAAA |
| | | ATGGTGCTTAAGTTCCAGCAGATGCCACATAAGGGGTTTGCCATTTGAT |
| | | ACCCCTGGGGAACATTTCTGTAAATACCATTGACACATCCGCCTTTTGT |
| | | ATACATCCTGGGTAATGAGAGGTGGCTTTTGCGGCCAGTATTAGACTG |
| | | GAAGTTCATACCTAAGTACTGTAATAATACCTCAATGTTTGAGGAGCA |
| | | TGTTTTGTATACAAATATATTGTTAATCTCTGTTATGTACTGTACTAATT |
| | | CTTACACTGCCTGTATACTTTAGTATGACGCTGATACATAACTAAATTT |
| | | GATACTTATATTTTCGTATGAAAATGAGTTGTGAAAGTTTTGAGTAGAT |
| | | ATTACTTTATCACTTTTTGAACTAAGAAACTTTTGTAAAGAAATTTACT |
| | | ATATATATATGCCTTTTTCCTAGCCTGTTTCTTCCTGTTAATGTATTTGT |
| | | TCATGTTTGGTGCATAGAACTGGGTAAATGCAAAGTTCTGTGTTTAATT |
| | | TCTTCAAAATGTATATATTTAGTGCTGCATCTTATAGCACTTTGAAATA |
| | | CCTCATGTTTATGAAAATAAATAGCTTAAAATTAAA |
| SEQ ID | A 20 nt reverse | CCGAACUCGACAGGGGACCG |
| NO: 18 | compliment RNA of | |
| | N-myc | |
| | for engineered | |
| | binding site | |
| SEQ ID | Survivin cDNA | GCGGCGCGCCATTAACCGCCAGATTTGAATCGCGGGACCCGTTGGCAG |
| NO: 19 | GenBank Accession | AGGTGGCGGCGGCGGCATGGGTGCCCCGACGTTGCCCCCTGCCTGGCA |
| | Number: | GCCCTTTCTCAAGGACCACCGCATCTCTACATTCAAGAACTGGCCCTTC |
| | NM_001012270.2 | TTGGAGGGCTGCGCCTGCACCCCGGAGCGGATGGCCGAGGCTGGCTTC |
| | | ATCCACTGCCCCACTGAGAACGAGCCAGACTTGGCCCAGTGTTTCTTCT |
| | | GCTTCAAGGAGCTGGAAGGCTGGGAGCCAGATGACGACCCCATGCAA |
| | | AGGAAACCAACAATAAGAAGAAAGAATTTGAGGAAACTGCGGAGAAA |
| | | GTGCGCCGTGCCATCGAGCAGCTGGCTGCCATGGATTGAGGCCTCTGG |
| | | CCGGAGCTGCCTGGTCCCAGAGTGGCTGCACCACTTCCAGGGTTTATTC |
| | | CCTGGTGCCACCAGCCTTCCTGTGGGCCCCTTAGCAATGTCTTAGGAAA |
| | | GGAGATCAACATTTTCAAATTAGATGTTTCAACTGTGCTCTTGTTTTGT |
| | | CTTGAAAGTGGCACCAGAGGTGCTTCTGCCTGTGCAGCGGGTGCTGCT |
| | | GGTAACAGTGGCTGCTTCTCTCTCTCTCTCTCTTTTTTGGGGGCTCATTT |
| | | TTGCTGTTTTGATTCCCGGGCTTACCAGGTGAGAAGTGAGGGAGGAAG |
| | | AAGGCAGTGTCCCTTTTGCTAGAGCTGACAGCTTTGTTCGCGTGGGCAG |
| | | AGCCTTCCACAGTGAATGTGTCTGGACCTCATGTTGTTGAGGCTGTCAC |
| | | AGTCCTGAGTGTGGACTTGGCAGGTGCCTGTTGAATCTGAGCTGCAGG |
| | | TTCCTTATCTGTCACACCTGTGCCTCCTCAGAGGACAGTTTTTTTGTTGT |
| | | TGTGTTTTTTTGTTTTTTTTTTTTTGGTAGATGCATGACTTGTGTGTGAT |
| | | GAGAGAATGGAGACAGAGTCCCTGGCTCCTCTACTGTTTAACAACATG |
| | | GCTTTCTTATTTTGTTTGAATTGTTAATTCACAGAATAGCACAAACTAC |
| | | AATTAAAACTAAGCACAAAGCCATTCTAAGTCATTGGGGAAACGGGGT |
| | | GAACTTCAGGTGGATGAGGAGACAGAATAGAGTGATAGGAAGCGTCT |
| | | GGCAGATACTCCTTTTGCCACTGCTGTGTGATTAGACAGGCCCAGTGA |
| | | GCCGCGGGGCACATGCTGGCCGCTCCTCCCTCAGAAAAAGGCAGTGGC |
| | | CTAAATCCTTTTTAAATGACTTGGCTCGATGCTGTGGGGGACTGGCTGG |
| | | GCTGCTGCAGGCCGTGTGTCTGTCAGCCCAACCTTCACATCTGTCACGT |
| | | TCTCCACACGGGGGAGAGACGCAGTCCGCCCAGGTCCCCGCTTTCTTT |
| | | GGAGGCAGCAGCTCCCGCAGGGCTGAAGTCTGGCGTAAGATGATGGAT |
| | | TTGATTCGCCCTCCTCCCTGTCATAGAGCTGCAGGGTGGATTGTTACAG |
| | | CTTCGCTGGAAACCTCTGGAGGTCATCTCGGCTGTTCCTGAGAAATAA |
| | | AAAGCCTGTCATTTCAAACACTGCTGTGGACCCTACTGGGTTTTTAAAA |
| | | TATTGTCAGTTTTTCATCGTCGTCCCTAGCCTGCCAACAGCCATCTGCC |
| | | CAGACAGCCGCAGTGAGGATGAGCGTCCTGGCAGAGACGCAGTTGTCT |
| | | CTGGGCGCTTGCCAGAGCCACGAACCCCAGACCTGTTTGTATCATCCG |
| | | GGCTCCTTCCGGGCAGAAACAACTGAAAATGCACTTCAGACCCACTTA |
| | | TTTCTGCCACATCTGAGTCGGCCTGAGATAGACTTTTCCCTCTAAACTG |
| | | GGAGAATATCACAGTGGTTTTTGTTAGCAGAAAATGCACTCCAGCCTC |
| | | TGTACTCATCTAAGCTGCTTATTTTTGATATTTGTGTCAGTCTGTAAATG |
| | | GATACTTCACTTTAATAACTGTTGCTTAGTAATTGGCTTTGTAGAGAAG |
| | | CTGGAAAAAAATGGTTTTGTCTTCAACTCCTTTGCATGCCAGGCGGTGA |
| | | TGTGGATCTCGGCTTCTGTGAGCCTGTGCTGTGGGCAGGGCTGAGCTG |
| | | GAGCCGCCCCTCTCAGCCCGCCTGCCACGGCCTTTCCTTAAAGGCCATC |
| | | CTTAAAACCAGACCCTCATGGCTACCAGCACCTGAAAGCTTCCTCGAC |
| | | ATCTGTTAATAAAGCCGTAGGCCCTTGTCTAAGTGCAACCGCCTAGACT |
| | | TTCTTTCAGATACATGTCCACATGTCCATTTTTCAGGTTCTCTAAGTTGG |
| | | AGTGGAGTCTGGGAAGGGTTGTGAATGAGGCTTCTGGGCTATGGGTGA |
| | | GGTTCCAATGGCAGGTTAGAGCCCCTCGGGCCAACTGCCATCCTGGAA |
| | | AGTAGAGACAGCAGTGCCCGCTGCCCAGAAGAGACCAGCAAGCCAAA |
| | | CTGGAGCCCCCATTGCAGGCTGTCGCCATGTGGAAAGAGTAACTCACA |
| | | ATTGCCAATAAAGTCTCATGTGGTTTTATCTA |
| SEQ ID | A 20 nt reverse | CUCUGCCAGGACGCUCAUCC |
| NO: 20 | compliment RNA of | |
| | Survivin | |
| | for engineered | |
| | binding site | |
| SEQ ID | AKT2 cDNA | GCCGTGCTAGCCGTTGGGCCTGCCTCGGAGGAGGCGTCGCCGCCGCCG |
| NO: 21 | GenBank Accession | CTGCCGCTGCCGGCGCCGTTGCCGCTGCCGGGAAACACAAGGAAAGGG |
| | Number: | AACCAGCGCAGCGTGGCGATGGGCGGGGGTAGAGCCCCGCCGGAGAG |
| | NM_001626.6 | GCTGGGCGGCTGCCGGTGACAGACTGTGCCCTGTCCACGGTGCCTCCT |
| | | GCATGTCCTGCTGCCCTGAGCTGTCCCGAGCTAGGTGACAGCGTACCA |
| | | CGCTGCCACCATGAATGAGGTGTCTGTCATCAAAGAAGGCTGGCTCCA |
| | | CAAGCGTGGTGAATACATCAAGACCTGGAGGCCACGGTACTTCCTGCT |
| | | GAAGAGCGACGGCTCCTTCATTGGGTACAAGGAGAGGCCCGAGGCCCC |
| | | TGATCAGACTCTACCCCCCTTAAACAACTTCTCCGTAGCAGAATGCCAG |
| | | CTGATGAAGACCGAGAGGCCGCGACCCAACACCTTTGTCATACGCTGC |
| | | CTGCAGTGGACCACAGTCATCGAGAGGACCTTCCACGTGGATTCTCCA |
| | | GACGAGAGGGAGGAGTGGATGCGGGCCATCCAGATGGTCGCCAACAG |
| | | CCTCAAGCAGCGGGCCCCAGGCGAGGACCCCATGGACTACAAGTGTGG |
| | | CTCCCCCAGTGACTCCTCCACGACTGAGGAGATGGAAGTGGCGGTCAG |
| | | CAAGGCACGGGCTAAAGTGACCATGAATGACTTCGACTATCTCAAACT |
| | | CCTTGGCAAGGGAACCTTTGGCAAAGTCATCCTGGTGCGGGAGAAGGC |
| | | CACTGGCCGCTACTACGCCATGAAGATCCTGCGGAAGGAAGTCATCAT |
| | | TGCCAAGGATGAAGTCGCTCACACAGTCACCGAGAGCCGGGTCCTCCA |
| | | GAACACCAGGCACCCGTTCCTCACTGCGCTGAAGTATGCCTTCCAGAC |
| | | CCACGACCGCCTGTGCTTTGTGATGGAGTATGCCAACGGGGGTGAGCT |
| | | GTTCTTCCACCTGTCCCGGGAGCGTGTCTTCACAGAGGAGCGGGCCCG |
| | | GTTTTATGGTGCAGAGATTGTCTCGGCTCTTGAGTACTTGCACTCGCGG |
| | | GACGTGGTATACCGCGACATCAAGCTGGAAAACCTCATGCTGGACAAA |
| | | GATGGCCACATCAAGATCACTGACTTTGGCCTCTGCAAAGAGGGCATC |
| | | AGTGACGGGGCCACCATGAAAACCTTCTGTGGGACCCCGGAGTACCTG |
| | | GCGCCTGAGGTGCTGGAGGACAATGACTATGGCCGGGCCGTGGACTGG |
| | | TGGGGGCTGGGTGTGGTCATGTACGAGATGATGTGCGGCCGCCTGCCC |
| | | TTCTACAACCAGGACCACGAGCGCCTCTTCGAGCTCATCCTCATGGAA |
| | | GAGATCCGCTTCCCGCGCACGCTCAGCCCCGAGGCCAAGTCCCTGCTT |
| | | GCTGGGCTGCTTAAGAAGGACCCCAAGCAGAGGCTTGGTGGGGGGCCC |
| | | AGCGATGCCAAGGAGGTCATGGAGCACAGGTTCTTCCTCAGCATCAAC |
| | | TGGCAGGACGTGGTCCAGAAGAAGCTCCTGCCACCCTTCAAACCTCAG |
| | | GTCACGTCCGAGGTCGACACAAGGTACTTCGATGATGAATTTACCGCC |
| | | CAGTCCATCACAATCACACCCCCTGACCGCTATGACAGCCTGGGCTTA |
| | | CTGGAGCTGGACCAGCGGACCCACTTCCCCCAGTTCTCCTACTCGGCC |
| | | AGCATCCGCGAGTGAGCAGTCTGCCCACGCAGAGGACGCACGCTCGCT |
| | | GCCATCACCGCTGGGTGGTTTTTTCCCCCTAACTTTTTACTTAGCCTTTT |
| | | TGGTTTGTGTCCCCACCCCCACCTCCTCACCCCCTTTCCAGTTCTTCTTC |
| | | AGGCCCCTCCCAGACGCACCCCAGCGGCCCCTGCAGCCCCTGCCTCCA |
| | | GCCTCCAGCCTCACCTTTGTGCCCAGACTCGCATTTGGAAGACTCCACC |
| | | TCCCGCCCAGGCCTGGGCTGTTGGGCGGTTGGAGATTCAGGTTTTAATC |
| | | CACACAAGCCCCAGTGAGGGGTGAAGCATGGCGCCTGGGGCCTGCCTG |
| | | AGTTTCTGGCCTGGGTGTCGTGCTGGTGTCTGCCTCCGCGCTGCTGCAT |
| | | CTGGACGAAGGCTGCCTTCTGGTGGGACGCGACACCCGGCAGACAGTG |
| | | GTGCTGCCTTCCAGGCCCCGTGGCCTAGGCTCGGAGTGGCCAGGCACG |
| | | GGGCGGTCCAATCCCCCACCCGCTGTCCCCCTATGGGGGCAGAAAAGC |
| | | AATAATGTCCAGGGGCAGGCAGGGGCCCTTGGGAGCTGCAGGGCTGG |
| | | GGGTTAGGGCTGCTCCCTGGTGAATGGAGTCAGATCCTAGGATCTGTA |
| | | CCATGGGGAACCAGGAGTGGCCGGGCTGGGTGCCGCCTCCTGGTCCGG |
| | | CCTCCTCCCCACCAAACTGTCCTCACCCTATGGATGAGGCAGGAGGAA |
| | | CATTTGGGGCCAAACCTGCCTGCCTCCCAGCCCCGTGCCTTACTAGGGC |
| | | TTCCTTCCAGCTGGCCTTACCTCCCGCTGGACCCTGGGCCTGGCCTGGC |
| | | CCCACTGGGGGCTATGGGCTGGGCTCACCCTCTCCTCTGCGGGGGTGG |
| | | AGGGCCACCAGCCTTGGCTGTTACAATCTTACACCGGACAGTATTGGG |
| | | CCCCATGGACTTGGTCAGGGAGGGGTGGGGGGGGCATCTCTGGTACC |
| | | TATTGGGGTGGGGGGCCTCTGAAAAGGGAGGCTCCTAGGCCCCCCTCA |
| | | CCCCTCCCTCTCCCCAGGGCCCCACGTTCTGCAGCCTTAAGGTTGAACA |
| | | TGAGTGCACGTCCATGTCAGTGCTGTGGGACTCCTGTGCGTGCCTCGGA |
| | | CTGCGTGTGTCGGCGGGACGCAGGCACACGTGGGTGTGTGTGCATGTG |
| | | TGTTTGTGTGAGGGCAGCGTGTCCTCCAGTGTGCATGGTGTGTGGGCTT |
| | | GGGCCCCATCCCTGGCCCGAGCATTTCATCCTGTGGGGGAGGGGTGCT |
| | | GACCTAGTGGGAGGAGCCCCACTGTGATCCATGAGCTGCCCTGCCCAC |
| | | GCCTCCCCTCCCTGTAGCAACACCTCTGGGTGTTTGGAGTTTAGCTTTT |
| | | GTGGGTTTGCTCTCCCTATCCCATCTCCTGTACTACACAGTTCATGGCA |
| | | GGGTGGGGAGGGGTGGGGTTGGTTCGGGGGGTGAGGGTCTTTTTCCT |
| | | CTGTGTGCGATGTTGTTATCTGACAGTTCTCCGTCCCTACTGGCCTTTCT |
| | | CCTCGTCTTCATATTTGTACGGTACAAGCAATAAAGACACTCATTTCAG |
| | | ACCAGGGCCCAGCCTGCACTCACGCCAGCCCAACCACTCTGGGCTTTG |
| | | CCTTGGTGATGGAGTCAGACCCCTGGGCCCCAGCTCCTCCTGTACTAGC |
| | | CGTTCCCTTCAGCAAGGAGGGCACTGAGCTCAGGGTGAGGGCAGCTGG |
| | | GGTGTGTGCAGGAGCTCAGGCTGGAGAGGGTGGGTGGAGCTGGTGCTG |
| | | TGGGGCTGAGGGGTATGGGAAGGCTCCCCGCATGTGGGGGTGGGGTG |
| | | GACAGAGACCACTCCAGGCCCTCAGTGCTGCTTAGGCTAAGAGAGGTG |
| | | GGGTGGAGGGACAGGGCTGGAAGATCTGGGTAGCCCAGAATGAGGAG |
| | | GGTGCCTGTGCTGTCACTGAATGAGAGGGAGTGGTTCATTCCACCCGG |
| | | CTGCCGAGCCTCAGAGGGGGGCATTCCTATCCTGCCCCACCTCCCTGTT |
| | | TATGCTGCCACCTGGAAGCCTTGAGGCCCCCAAATTCCAGTACAGACC |
| | | CAGTGGTGTGTTCATGGTGGCGTGGTTGCTGTCACCTGGGAGCTCCTGA |
| | | GCGTTTGGTTAGAACCCTGTTCAGCTTGGGGTCAGCCCTCCCCTAGTCA |
| | | CTGCCCTTTAGCCTGGATGTGTCTGGGCCCCTGCACTTCCCGTGCTTGA |
| | | GTCACGTGGCTGCATGGCCGGGCGCTGGCCGGATGGAACACCTCCCCC |
| | | AGCAAGGGACCAGGGACCAGAGCCCTGGCCTGCCCTGCTGAGCCCTGC |
| | | TGTGCAGAGGGCCTGGCACAGATGAATTTGAGATTTTGCCGCAAGGTG |
| | | TTAGCACTTCACACCCATTGAGTCTTTGAGATTTTAAGTGAATGTAAGC |
| | | AGAAAAAGTCAGATCCAATTTACAGAAATCAGAGTTAGCTACAGCTAG |
| | | GACTCGTTTGGTTGGGGTTTTTTAGTTTGTCTTTCTAAAGTCATGTGGAC |
| | | CTTAATTTAATTACAAAAGTCTACCCTGGTGGTCATAAAATAGGCAGG |
| | | CCTATGAAGAAAGGCCTTTTACTCTTCCATCTCGTCCCAGCCCCGAGTT |
| | | GACCCACGTTGCTGCTCCTCACACCATGGTGATGCAGGTCTCGTAGTGT |
| | | GGGCACAGGCCTGGCTACCTCATCTTTTTAGTGCCTCTCTCCTCTTCCA |
| | | CAGGATGGGGTCCCACAGCTGCAGCAGCTGGCCCCGTAGTTGAGCATG |
| | | TGTGGTTATCCTGTAGAGCTTTTCCCAAGAAGGGTGTTTGAACTTAGAG |
| | | TCTTAATAAAATCTTACCAAATAAATTTTGAGTAGAATAATCGTCTTTT |
| | | GCAATGTACATTTTAAAAATTTCACACATTCTTTTTTGTATATAAAGAA |
| | | CAGTGACTGGGCACAGTGGCTCATGCCTGTAATCCCAGCAATTTGGGA |
| | | GGCCGAGGCGGGCGGGTCTCTTGAGGCCAGGGGTTCGAGACCAGCCTG |
| | | GGCATCATAGGGAGACCTTCATCTCTACAAAAAATACAAAAATTAGCT |
| | | GGGCATGGTGGTGCATGCCTGCAATCCCAGCTAACTTGGAAGGCTGAG |
| | | GTGAGGTGGGAAGATCACTTGAGCCCAGGAGTTTGAGGCTGCAGTGAG |
| | | CTATGATTGCGGCACTGCACTGCAGCCTGGGACAATGAGACTGTGTCT |
| | | CTAAAAATAAAAAAAAAAAAAACATGATACATGCTATTAAAAAAGAC |
| | | AGCAAAGCAGGAGTATAAGAAAGGAAATTCACCCGAGGTCGCAGGGC |
| | | CTTGAGTACTCATTTTGGTGCTGATTACCTCTCTGCAAATGGACACGGC |
| | | ATCATAAATTGGTAGTTTCCTGCTCTTTTTGTGTAATCTTTTCCAGTTAA |
| | | TGTGAAGCCTCTGGGGGCTGCCCTCGTGCACTGATGGTTGTGTGGAGTC |
| | | GGGGGCGGCAGTGCGATTCCCTTTTAGCTGCTGCATGGGGGGAACTCA |
| | | GGCTTTCCAGCTGCTTCCTGGGGTTCCATGGGGTAGACCCCTCAACCGC |
| | | TTCAGCTGCCCCGTTAACAGGAATTGACTTGGTTTCGTTTGGTGCTACC |
| | | AGCAGTCCTGTAATAAACTAGCTATCCATCTGTA |
| SEQ ID | A 20 nt reverse | AGCUCCUGCUCACACCCCAG |
| NO: 22 | compliment RNA of | |
| | AKT2 | |

### Bacterial Infections

Bacterial infections may be caused by, but not limited to: Acetobacter aurantius, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia, Burkholderia mallei, Burkholderia pseudomallei, Burkholderia cepacian, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetiid, Ehrlichia chaffeensis, Ehrlichia ewingii, Eikenella corrodens, Enterobacter cloacae, Enterococcus avium, Enterococcus durans, Enterococcus faecalis, Enterococcus faecium, Enterococcus gallinarum, Enterococcus maloratus, Escherichia coli, Fusobacterium necrophorum, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Legionella pneumophila, Leptospira interrogans, Leptospira noguchii, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma Mexican, Neisseria gonorrhoeae, Neisseria meningitidis, Pasteurella multocida, Pasteurella tularensis, Porphyromonas gingivalis, Prevotella melaninogenica Pseudomonas aeruginosa, Rhizobium radiobacter, Rickettsia prowazekii, Rickettsia psittaci, Rickettsia quintana, Rickettsia rickettsia, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Ureaplasma urealyticum, Vibrio cholerae, Vibrio comma, Vibrio parahaemolyticus, Vibrio vulnificus, Yersinia enterocolitica, Yersinia pestis, and Yersinia pseudotuberculosis

### β-Lactamase Resistant Bacterial Infections

In certain instances, regarding the treatment of a bacterial infection, the infection may include bacteria which have become resistant to one or multiple types of antibiotics. Examples of bacteria that have become resistant to antibiotics include methicillin-resistant Staphylococcus aureus (MRSA), vancomycin-resistant Enterococcus (VRE), multi-drug-resistant Mycobacterium tuberculosis (MDR-TB), carbapenem-resistant Enterobacteriaceae (CRE) gut bacteria, cephalosporin resistant bacteria, carbapenem resistant bacteria, and ciprofloxacin resistant bacteria.

The β-lactam family of antibiotic molecules may comprise cephalosporins, monobactam, penicillins, and carbapenems. Cephalosporins, like other β-lactams, may bind to the bacterial penicillin-binding proteins (PBPs). Resistance may arise when the PBDs are protected by beta-lactamases. Beta-lactamases are produced widely by bacteria and may be encoded by chromosomal or plasmid DNA.

Drug resistance to cephalosporins or carbapenem may develop and transfer β-lactam resistance (including carbapenem resistance) in many ways. For example, they may generate new extended-spectrum β-lactamases (ESBL) from the existing spectrum of plasmid-mediated β-lactamases through amino acid substitution. They may acquire genes encoding β-lactamases from environmental bacteria. They may increase the expression of chromosome-encoded β-lactamase genes (bla genes) due to regulatory gene and promoter sequence modifications. Alternately or additively, they can mobilize bla genes through integrons or horizontal transfer of genomic islands into other gram-negative species and strains. Further, bacteria can disseminate plasmid-mediated β-lactamases.

Types of β-lactamases include but are not limited to the following: extended-spectrum β-lactamase (ESBL), TEM β-lactamases, SHV β-lactamases, CTX-M β-lactamases, OXA beta-lactamases, IMP-type carbapenemases (metallo-β-lactamases) (class B), VIM (Verona integron-encoded metallo-β-lactamase) (Class B), and OXA (oxacillinase) group of β-lactamases (class D).

An engineered RNA of the present disclosure may help to downregulate β-lactamases by complementary binding between a portion of an mRNA encoding one or more β-lactamases and a binding site of an engineered RNA. The binding site may be 70% complementary to a sequence found in an RNA encoding a β-lactamase enzyme. In other instances, the binding site may exhibit at or over: 70 percent, 80 percent, 85 percent, 90 percent, 95 percent or more complementary binding between a binding site of an engineered nucleic acid and a target sequence of an RNA encoding a β-lactamase enzyme. In some cases, the binding site may exhibit perfect complementary binding between the engineered nucleic acid and the target sequence. Still further, an engineered RNA may downregulate multiple classes of β-lactamases through multiple binding sites. In some instances, an engineered RNA may independently comprise loops with at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-identical binding sites. In certain instances, the non-identical binding sites may be directed to the same RNA encoding β-lactamase with different affinities. In instances, the non-identical binding sites may be directed to bind RNA encoding multiple different β-lactamases. In some cases, the engineered polynucleotide may have binding sites within the loops of its stem loops in which the engineered polynucleotide binds two or more different RNA sequences for genes encoding β-lactamases with different binding affinities.

β-lactamase

A DNA sequence corresponding to an RNA encoding a TEM-1 β-lactamase may be found on GenBank with accession number KP634897.1 and is listed in Table 4 paired with an example of a binding site having a reverse complement to a portion of RNA sequence for the TEM-1 β-lactamase listed. See SEQ. ID. NOS: 23, 24.

A DNA sequence corresponding to an RNA encoding a SHV β-lactamase may be found on GenBank with accession number KX171170.1 and is listed in Table 4 paired with an example of a binding site having a reverse complement to a portion of RNA sequence for the SHV β-lactamase listed. See SEQ. ID. NOS: 25, 26.

A DNA sequence corresponding to an RNA encoding a CTX-M β-lactamase may be found on GenBank with accession number DNA sequences corresponding to an RNA encoding a CTX-M β-lactamase may be found on GenBank with accession number MH814718.1 and is listed in Table 4 paired with an example of a binding site having a reverse complement to a portion of RNA sequence for the CTX-M β-lactamase listed. See SEQ. ID. NOS: 27, 28.

A DNA sequence corresponding to an RNA encoding a OXA β-lactamase may be found on GenBank with accession number DNA sequences corresponding to an RNA encoding a OXA β-lactamase may be found on GenBank with accession number KX171195.1 and is listed in Table 4 paired with an example of a binding site having a reverse complement to a portion of RNA sequence for the OXA β-lactamase listed. See SEQ. ID. NOS: 29, 30.

A DNA sequence corresponding to an RNA encoding a ESBL β-lactamase may be found on GenBank with accession number DNA sequences corresponding to an RNA encoding a ESBL β-lactamase may be found on GenBank with accession number LC440650.1 and is listed in Table 4 paired with an example of a binding site having a reverse complement to a portion of RNA sequence for the ESBL β-lactamase listed. See SEQ. ID. NOS: 31, 32.

A DNA sequence corresponding to an RNA encoding an IMP carbapenemase may be found on GenBank with accession number DNA sequences corresponding to an RNA encoding an IMP carbapenemase may be found on GenBank with accession number HQ263342.1 and is listed in Table 4 paired with an example of a binding site having a reverse complement to a portion of RNA sequence for the IMP carbapenemase listed. See SEQ. ID. NOS: 33, 34.

A DNA sequence corresponding to an RNA encoding a VIM (Verona integron-encoded metallo-β-lactamase) may be found on GenBank with accession number DNA sequences corresponding to an RNA encoding a VIM (Verona integron-encoded metallo-β-lactamase) may be found on GenBank with accession number AF531419.1 and is listed in Table 4 paired with an example of a binding site having a reverse complement to a portion of RNA sequence for the VIM (Verona integron-encoded metallo-β-lactamase) listed. See SEQ. ID. NOS: 35, 36.

A DNA sequence corresponding to an RNA encoding an oxacillinase may be found on GenBank with accession number DNA sequences corresponding to an RNA encoding an oxacillinase may be found on GenBank with accession number AY306135.1 and is listed in Table 4 paired with an example of a binding site having a reverse complement to a portion of RNA sequence for the oxacillinase listed. See SEQ. ID. NOS: 37, 38.

### Ciprofloxacin Resistant Bacterial Infections

Ciprofloxacin is an antimicrobial of the fluoroquinolone class. The substrate of ciprofloxacin is the complex formed by the DNA of the bacterium and either the DNA gyrase enzyme or the topoisomerase IV enzyme. DNA gyrase creates single-stranded breaks in the DNA to supercoil negatively the DNA during replication or transcription. If ciprofloxacin binds DNA gyrase in complex with DNA, the single-stranded DNA breaks cannot be re-ligated and thus accumulate, leading to double-stranded DNA breaks. Genes known to play a role in ciprofloxacin resistance include: gyrA, gyrB, parC, marR, acrRAB, tolC, soxS, rpoB, qepA, oqxAB, qnrA, qnrB, qnrC, qnrD, qnrE, qnrS, and crpP.

An engineered RNA of the present disclosure may help to downregulate a protein conferring ciprofloxacin resistance by complementary binding between a portion of an mRNA encoding one or more genes conferring ciprofloxacin resistance and a binding site of an engineered RNA. The binding site may be 70% complementary to a sequence found in an RNA encoding a β-lactamase enzyme. In other instances, the binding site may exhibit at or over: 70 percent, 80 percent, 85 percent, 90 percent, 95 percent or more complementary binding between a binding site of an engineered nucleic acid and a target sequence of an RNA encoding a β-lactamase enzyme. In some cases, the binding site may exhibit perfect complementary binding between the engineered nucleic acid and the target sequence. Still further, an engineered RNA may downregulate multiple genes encoding proteins conferring ciprofloxacin through multiple binding sites. In some instances, an engineered RNA may comprise loops with at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-identical binding sites. In certain instances, the non-identical binding sites may be directed to the same RNA encoding genes conferring ciprofloxacin resistance with different affinities. In some instances, the non-identical binding sites may be directed to bind RNA encoding multiple different genes conferring ciprofloxacin resistance. In some cases, the engineered polynucleotide may have binding sites within the loops of its stem loops in which the engineered polynucleotide binds two or more different RNA sequences encoding for proteins conferring ciprofloxacin resistance with different binding affinities.

A DNA sequence corresponding to a selected gene encoding a protein which confers ciprofloxacin resistance, namely parC may be found on GenBank with accession number KU521518.1 and is listed in Table 4 paired with an example of a binding site having a reverse complement to a portion of RNA sequence for the parC gene listed. See SEQ. ID. NOS: 39, 40.

### Bacterial Metabolic Pathway

In some instances, an engineered RNA can comprise three or more binding sites individually within loops of the stem loops of the engineered RNA. In certain instances, the engineered polynucleotide binding sites each bind to or hybridize with a target RNA sequence encoding a protein such that when the individual RNAs encoding proteins are bound to or hybridized with the engineered polynucleotide binding sites, a bacterial metabolic pathway is disrupted. In certain instances, the metabolic pathway may comprise a protein involved in a metabolic pathway. In certain instances, the protein comprises PBP1a/b, PBP4, PBP2c, PBP2d, PBP2a, PbpH, PBP2b, PBP3, SpoVD, PBP4b, PBP5, PBP4a, DacF, PbpX, MepA, AmpC, AmpH or any combination thereof. In certain instances, the metabolic pathway can control the shape and/or structure of a bacteria. In certain instances, wherein the metabolic pathway controls the shape of a bacteria, the proteins comprise PBP4, PBP5, PBP7, AmpC, AmpH, or any combination thereof.

### Fungal Infections

In some aspects, a disease can be caused by a fungal infection. In some aspects, a fungal disease can be blastomycosis which may be caused by the fungus Blastomyces. In some aspects, a fungal disease is coccidioidomycosis which may be caused the fungus Coccidioides. In some instances, the fungal disease is a cryptococcosis infection caused by Cryptococcus gattii. In some instances, the fungal disease is histoplasmosis caused by the fungus histoplasma. In some instances, the fungal infection is paracoccidioidomycosis caused by the fungus Paracoccidioides. In some instances, the fungal disease is aspergillosis caused by the fungus Aspergillus. In some instances, the fungal disease is candidiasis caused by the fungus candida, in some instances, the fungal disease is caused by Cryptococcus neoformans. In some instances, the fungal disease is mucormycosis caused by mucormycetes molds. In some instances, the fungal disease is talaromycosis caused by Talaromyces marneffei.

Candida albicans Fungal Disease

In certain instances, regarding the treatment of a fungal infection, the infection may include a fungus derived toxin such as candidalysin. This toxin is a 31 amino acid peptide toxin derived from the gene ECE1 of C. albicans and has a role in inflammasome activation and induction of cell damage.

A DNA sequence corresponding to an RNA encoding the ECE1 protein may be found on GenBank with accession number L17087.1 and is listed in Table 4 paired with an example of a binding site having a reverse complement to a portion of RNA sequence for the ECE1 listed. See SEQ. ID. NOS: 45, 46.

Potential target sequences or genes encoding target sequences for engineered nucleic acids disclosed herein are shown in Table 4. Any DNA sequence provided herein also includes the RNA sequence wherein all T's are substituted for U's. Table 4 also provides reverse complement sequences that can be incorporated into a engineered nucleic acid design. In some cases, an engineered nucleic acid herein can comprise a sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 24, 26, 28, 30, 32, 34, 36, 38, 40, or 46.

TABLE 4
Potential target sequences of bacterial and fungal mediated diseases

| Sequence ID | Description | Sequence |
|---|---|---|
| SEQ ID NO: | ß-lactamase | CCCAGAAACGCTGGTGAAAGTAAAAGATGCTGAAGATCAGTTGGGTGCACGA |
| 23 | Tem-1 DNA | GTGGGTTACATCGAACTGGATCTCAACAGCGGTAAGATCCTTGAGAGTTTTCG |
| | sequence | CCCCGAAGAACGTTTTCCAATGATGAGCACTTTTAAAGTTCTGCTATGTGGTGC |
| | GenBank | GGTATTATCCCGTGTTGACGCCGGGCAAGAGCAACTCGGTCGCCGCATACACT |
| | Accession | ATTCTCAGAATGACTTGGTTGAGTACTCACCAGTCACAGAAAAGCATCTTACG |
| | Number: | GATGGCATGACAGTAAGAGAATTATGCAGTGCTGCCATAACCATGAGTGATAA |
| | KP634897.1 | CACTGCTGCCAACTTACTTCTGACAACGATCGGAGGACCGAAGGAGCTAACCG |
| | | CTTTTTTGCACAACATGGGGGATCATGTAACTCGCCTTGATCGTTGGGAACCGG |
| | | AGCTGAATGAAGCCATACCAAACGACGAGCGTGACACCACGATGCCTGCAGC |
| | | AATGGCAACAACGTTGCGCAAACTATTAACTGGCGAACTACTTACTCTAGCTTC |
| | | CCGGCAACAATTAATAGACTGGATGGAGGCGGATAAAGTTGCAGGACCACTTC |
| | | TGCGCTCGGCCCTTCCGGCTGGCTGGTTTATTGCTGATAAATCTGGAGCCGGTG |
| | | AGCGTGGGTCTCGCGGTATCATTGCAGCACTGGGGCCAGATGGTAAGCCCTCC |
| | | CGTATCGTAGTTATCTACACGACGGGGAGTCAGGCAACTATGGATGAACGAAA |
| | | TAGACAGATCGCTGAGATAGGTGCCTCACTGATAG |
| SEQ ID NO: | A 20 nt reverse | AUGGCUUCAUUCAGCUCCGG |
| 24 | compliment | |
| | RNA of DNA | |
| | encoding TEM-1 | |
| | ß-lactamase | |
| SEQ ID NO: | SHV ß- | ATGATAGAAATGGATCTGGCCAGCGGCCGCACGCTGACCGCCTGGCGCGCCGA |
| 25 | lactamase DNA | TGAACGCTTTCCCATGATGAGCACCTTTAAAGTAGTGCTCTGCGGCGCAGTGCT |
| | sequence | GGCGCGGGTGGATGCCGGTGACGAACAGCTGGAGCGAAAGATCCACTATCGC |
| | GenBank | CAGCAGGATCTGGTGGACTACTCGCCGGTCAGCGAAAAACATCTTGCCGACGG |
| | Accession | CATGACGGTCGGCGAACTCTGTGCCGCCGCCATTACCATGAGCGATAACAGCG |
| | Number: | CCGCCAATCTGCTGCTGGCCACCGTCGGCGGCCCCGCAGGATTGACTGCCTTTT |
| | KX171170.1 | TGCGCCAGATCGACGACAACGTCACCCGCCTTGACCGCTGGGAAACGGAACTG |
| | | AATGAGGCGCTTCCCGGCGACGCCCGCGACACCACTACCCCGGCCAGCATGGC |
| | | CGCGACCCTGCGCAAGCTGCTGACCAGCCAGCGTCTGAGCGCCCGTTCGCAAC |
| | | GGCAGCTGCTGCAGTGGATGGTGGACGATCGGGTCGCCGGACCGTTGATCCGC |
| | | TCCGTGCTGCCGGCGGGCTGGTTTATCGCCGATAAGACCGGAGCTGGCGAGCG |
| | | GGGTGCGCGCG |
| SEQ ID NO: | A 20 nt reverse | CCGGCAUCCACCCGCGCCAG |
| 26 | compliment | |
| | RNA of DNA | |
| | encoding SHV | |
| | ß-lactamase | |
| SEQ ID NO: | CTX-M ß- | ATGGTGACAAAGAGAGTGCAACGGATGATGTTCGCGGCGGCGGCGTGCATTCC |
| 27 | lactamase DNA | GCTGCTGCTGGGCAGCGCGCCGCTTTATGCGCAGACGAGTGCGGTGCAGCAAA |
| | sequence | AGCTGGCGGCGCTGGAGAAAAGCAGCGGAGGGCGGCTGGGCGTCGCGCTCAT |
| | GenBank | CGATACCGCAGATAATACGCAGGTGCTTTATCGCGGTGATGAACGCTTTCCAA |
| | Accession | TGTGCAGTACCAGTAAAGTTATGGCGGCCGCGGCGGTGCTTAAGCAGAGTGAA |
| | Number: | ACGCAAAAGCAGCTGCTTAATCAGCCTGTCGAGATCAAGCCTGCCGATCTGGT |
| | MH814718.1 | TAACTACAGTCCGATTGCCGAAAAACACGTCAACGGCACAATGACGCTGGCAG |
| | | AACTGAGCGCGGCCGTGTTGCAGTACAGCGACAATACCGCCATGAACAAATTG |
| | | ATTGCCCAGCTCGGTGGCCCGGGAGGCGTGACGGCTTTTGCCCGCGCGATCGG |
| | | CGATGAGACGTTTCGTCTGGATCGCACTGAACCTACGCTGAATACCGCCATTCC |
| | | CGGCGACCCGAGAGACACCACCACGCCGCGGGCGATGGCGCAGACGTTGCGT |
| | | CAGCTTACGCTGGGTCATGCGCTGGGCGAAACCCAGCGGGCGCAGTTGGTGAC |
| | | GTGGCTCAAAGGCAATACGACCGGCGCAGCCAGCATTCGGGCCGGCTTACCGA |
| | | CGTCGTGGACTGTGGGTGATAAGACCGGCAGCGGCGGCTACGGCACCACCAAT |
| | | GATATTGCGGTGATCTGGCCGCAGGGTCGTGCGCCGCTGGTTCTGGTGACCTAT |
| | | TTTACCCAGCCGCAACAGAACGCAGAGAGCCGCCGCGATGTGCTGGCTTCAGC |
| | | GGCGAGAATCATCGCCGAAGGGCTGTAA |
| SEQ ID NO: | A 20 nt reverse | ACGUCUGCGCCAUCGCCCGC |
| 28 | compliment | |
| | RNA of DNA | |
| | encoding CTX- | |
| | M ß-lactamase | |
| SEQ ID NO: | OXA ß- | AGGAACTGAAGGTTGTTTTTTACTTTACGATGCATCCACAAACGCTGAAATTGC |
| 29 | lactamase DNA | TCAATTCAATAAAGCAAAGTGTGCAACGCAAATGGCACCAGATTCAACTTTCA |
| | sequence | AGATCGCATTATCACTTATGGCATTTGATGCGGAAATAATAGATCAGAAAACC |
| | GenBank | ATATTCAAATGGGATAAAACCCCCAAAGGAATGGAGATCTGGAACAGCAATC |
| | Accession | ATACACCAAAGACGTGGATGCAATTTTCTGTTGTTTGGGTTTCGCAAGAAATAA |
| | Number: | CCCAAAAAATTGGATTAAATAAAATCAAGAATTATCTCAAAGATTTTGATTAT |
| | KX171195.1 | GGAAATCAAGACTTCTCTGGAGATAAAGAAAGAAACAACGGATTAACAGAAG |
| | | CATGGCTCGAAAGTAGCTTAAAAATTTCACCAGAAGAACAAATTCAATTCCTG |
| | | CGTAAAATTATTAATCACAATCTCCCAGTTAAAAACTCAGCCATAGAAAACAC |
| | | CATAGAGAACATGTATCTACAAGATCTGGATAATAGTACAAAACTGTATGGGA |
| | | AAACTGGTGCAGGATTCACAGCAAATAGAACCTTACAAAACGGATGGTTTGAA |
| | | GGGTTTATTATAAGCAAATCAGGACATAA |
| SEQ ID NO: | A 20 nt reverse | GAUUAAUAAUUUUACGCAGG |
| 30 | compliment | |
| | RNA of DNA | |
| | encoding OXA | |
| | ß-lactamase | |
| SEQ ID NO: | ESBL ß- | ATTAAACAAAGCGAAAGCCAGCTGTCGGGCCGCGTAGGCATGATAGAAATGG |
| 31 | lactamase DNA | ATCTGGCCAGCGGCCGCACGCTGACCGCCTGGCGCGCCGATGAACGCTTTCCC |
| | sequence | ATGATGAGCACCTTTAAAGTAGTGCTCTGCGGCGCAGTGCTGGCGCGGGTGGA |
| | GenBank | TGCCGGTGACGAACAGCTGGACGAAAGATCCACTATCGCCAGCAGGATCTGGT |
| | Accession | GGACTACTCGCCGGTCAGCGAAAAACACCTTGCCGACGGCATGACGGTCGGCG |
| | Number: | AACTCTGCGCCGCCGCCATTACCATGAGCGATAACAGCGCCGCCAATCTGCTG |
| | LC440650.1 | CTGGCCACCGTCGGCGGCCCCGCAGGATTGACTGCCTTTTTGCGCCAGATCGGC |
| | | GACAACGTCACCCGCCTTGACCGCTGGGAAACGGAACTGAATGAGGCGCTTCC |
| | | CGGCGACGCCCGCGACACCACTACCCCGGCCAGCATGGCCGCGACCCTGCGCA |
| | | AGCTGCTGACCAGCCAGCGTCTGAGCGCCGTTCGCAACGGCAGCTGCTGCAGT |
| | | GGATGGTGGACGATCGGGTCGCCGGACCGTTGATCCGCTCCGTGCTGCCGAAC |
| | | GGCGCTCAGACGCTGGGGCGGGCTGGTTTATCGCCGATAAGACCGGAGCTAGC |
| | | AGCGGGGTGCGCGCGGGATTGTC |
| SEQ ID NO: | A 20 nt reverse | GAACGGCGCUCAGACGCUGG |
| 32 | compliment | |
| | RNA of DNA | |
| | encoding ESBL | |
| | ß-lactamase | |
| SEQ ID NO: | IMP | ATGAGCAAGTTATTTGTATTCTTTATGTTTTTGTTTTGTAGCATTACTGCCGCAG |
| 33 | carbapenemase | CAGAGTCTTTGCCAGATTTAAAAATTGAGAGGCTTGATGAAGGCGTTTATGTTC |
| | DNA sequence | ATACTTCGTTTGAAGAAGTTAACGGTTGGGGTGTTGTTCCTAAACACGGCTTGG |
| | GenBank | TGGTTCTTGTAAATACTGAGGCCTATCTGATTGACACTCCATTTACGGCTAAAG |
| | Accession | ATACTGAAAAGTTAGTCACTTGGTTTGTGGGACGCGGCTATAAAATAAAAGGC |
| | Number: | AGTATTTCCTCTCATTTTCATAGCGACAGCACGGGCGGAATAGAGTGGCTTAAT |
| | HQ263342.1 | TCTCAATCTATCCCCACGTATGCATCTGAATTAACAAATGAACTTCTTAAAAAA |
| | | GACGGTAAGGTACAAGCTAAAAATTCATTTGGCGGAGTTAGCTATTGGCTAGT |
| | | TAAGAATAAGATTGAAGTTTTTTATCCTGGTCCAGGGCACACTCCAGATAACGT |
| | | AGTGGTTTGGCTACCTGAAAATAGAGTTTTGTTCGGTGGTTGTTTTGTTAAACC |
| | | GTACGGTCTTGGTAATTTGGGTGACGCAAATTTAGAAGCTTGGCCAAAGTCCG |
| | | CCAAATTATTAATGTCCAAATATGGTAAGGCAAAACTGGTTGTTCCAAGTCAC |
| | | AGTGAAGTTGGAGACGCATCACTCTTGAAGCGAACATTAGAACATGCGGTTAA |
| | | AGGGTTAAATGAAAGTAAAAAACCATCAAAACCAAGTAACTAA |
| SEQ ID NO: | A 20 nt reverse | CAAGUCACAGUGAAGUUGGA |
| 34 | compliment | |
| | RNA of DNA | |
| | encoding IMP | |
| | carbapenemase | |
| SEQ ID NO: | VIM (Verona | GTTATGCCGCACCCACCCCTATGGAGTCTTGATGTTAAAAGTTATTAGTAGTTT |
| 35 | integron- | ATTGGTCTACATGACCGCGTCTGTCATGGCTGTCGCAAGTCCGTTAGCCCATTC |
| | encoded | CGGGGAGCCGAGTGGTGAGTATCCGACAGTCAACGAAATTCCGGTCGGAGAG |
| | metallo-ß- | GTCCGACTTTACCAGATTGCCGATGGTGTTTGGTCGCATATCGCAACGCAGTCG |
| | lactamase) | TTTGATGGCGCGGTCTACCCGTCCAATGGTCTCATTGTCCGTGATGGTGATGAG |
| | DNA sequence | TTGCTTTTGATTGATACAGCGTGGGGTGCGAAAAACACAGCGGCACTTCTCGC |
| | GenBank | GGAGATTGAAAAGCAAATTGGACTTCCCGTAACGCGTGCAGTCTCCACGCACT |
| | Accession | TTCATGACGACCGCGTCGGCGGCGTTGATGTCCTTCGGGCGGCTGGGGTGGCA |
| | Number: | ACGTACGCATCACCGTCGACACGCCGGCTAGCCGAGGCAGAGGGGAACGAGA |
| | AF531419.1 | TTCCCACGCATTCTCTAGAAGGACTCTCATCGAGCGGGGACGCAGTGCGCTTC |
| | | GGTCCAGTAGAGCTCTTCTATCCTGGTGCTGCGCATTCGACCGACAATCTGGTT |
| | | GTATACGTCCCGTCAGCGAACGTGCTATACGGTGGTTGTGCCGTTCATGAGTTG |
| | | TCACGCACGTCTGCGGGGAACGTGGCCGATGCCGATCTGGCTGAATGGCCCAC |
| | | CTCCGTTGAGCGGATTCAAAAACACTACCCGGAAGCAGAGGTCGTCATTCCCG |
| | | GGCACGGTCTACCGGGCGGTCTAGACTTGCTCCAGCACACAGCGAACGTTGTC |
| | | AAAGCACACAAAAATCGCTCAGTCGCCGAGTAGCAGATGCGGCATAACAAAT |
| | | CGTTGGAGCGGGACTTTTGCTACGCAGGCTGCGCCTACTCCGCAAAAGCCCCT |
| | | CAACTCAGGC |
| SEQ ID NO: | A 20 nt reverse | GAAGGACAUCAACGCCGCCG |
| 36 | compliment | |
| | RNA of DNA | |
| | encoding VIM | |
| | (Verona | |
| | integron- | |
| | encoded | |
| | metallo-ß- | |
| | lactamase) | |
| SEQ ID NO: | Oxacillinase | ATGCGCCCTCTCCTCTTGAGTGCCCTTCTCCTGCTTTCCGGGCATACCCAGGCC |
| 37 | DNA sequence | AGCGAATGGAACGACAGCCAGGCCGTGGACAAGCTATTCGGCGCGGCCGGGG |
| | GenBank | TGAAAGGCACCTTCGTCCTCTACGATGTGCAGCGGCAGCGCTATGTCGGCCAT |
| | Accession | GACCGGGAGCGCGCGGAAACCCGCTTCGTTCCCGCTTCCACCTACAAGGTGGC |
| | Number: | GAACAGCCTGATCGGCTTATCCACAGGGGCGGTTAGATCCGCCGACGAGGTTC |
| | AY306135.1 | TTCCCTATGGCGGCAAGCCCCAGCGCTTCAAGGCCTGGGAGCACGACATGAGC |
| | | CTGCGCGAGGCGATCAAGGCATCGAACGTACCGGTCTACCAGGAACTGGCGCG |
| | | GCGCATCGGCCTGGAGCGGATGCGCGCCAATGTCTCGCGCCTGGGTTACGGCA |
| | | ACGCGGAAATCGGCCAGGTTGTGGATAACTTCTGGTTGGTGGGACCGCTGAAG |
| | | ATCAGCGCGATGGAACAGACCCACTTTCTGCTCCGACTGGCGCAGGGAGAATT |
| | | GCCATTCCCCGCCCCGGTGCAGTCCACCGTGCGCGCCATGACCCTGCTGGAAA |
| | | GCGGCCCGGGCTGGGAGCTGCACGGCAAGACCGGCTGGTGCTTCGACTGCACG |
| | | CCGGAACTCGGCTGGTGGGTGGGCTGGGTGAAGCGCAACGAGCGGCTCTACGG |
| | | CTTCGCCCTGAACATCGACATGCCCGGCGGCGAGGCCGACATCGGCAAGCGCG |
| | | TCGAACTGGGCAAGGCCAGTCTCAAGGCTCTCGGGATACTGCCCTGA |
| SEQ ID NO: | A 20 nt reverse | CCAGCAGGGUCAUGGCGCGC |
| 38 | compliment | |
| | RNA of DNA | |
| | encoding | |
| | Oxacillinase | |
| SEQ ID NO: | parC DNA | GCCGCATATCGGCGACGGCCTGAAGCCGGTGCAGCGACGCATCGTCTACGCCA |
| 39 | sequence | TGAGCGAACTGGGGCTGGATGCCGATTCCAAGCACAAGAAGTCGGCGCGCACC |
| | GenBank | GTCGGCGACGTGCTCGGCAAGTTCCACCCGCACGGCGATTCGGCCTGCTACGA |
| | Accession | GGCCATGGTGCTGATGGCGCAGCCGTTCTCCTATCGCTATCCGCTGGTGGACGG |
| | Number: | CCAGGGCAACTGGGGGGCTCC |
| | KU521518.1 | |
| SEQ ID NO: | A 20 nt reverse | CAUGGCGUAGACGAUGCGUC |
| 40 | compliment | |
| | RNA of a parC | |
| | DNA sequence | |
| SEQ ID NO: | ECE1 protein | TTTTGGAGATAACCACTAGCGAAGTAAGACATATTACAAAAATTTCCAGCCAC |
| 45 | DNA sequence | TATTTTGTACCTGTAACTTTCAAAAATGATTACACTTTTTGACTTCGTTATCAGT |
| | from Candida | GGCTTCATATTATATAGTACTTATCAAAGCTTTAATACTTTTGAGGTATCATAA |
| | albicans | CCTTAAGAAATTGTTTTCAATCTATTACTCGTCTCACACGGTTAGAAGTCATTT |
| | GenBank | GTAGGATTTTCAGCAGAACAACCATTTTTGTTGTATAGAAGATAGACGCCAAG |
| | Accession | AAAAAAACAAGAGTACGTTATACTTTGAACAAGATACACAAAGAGTTAAATAC |
| | Number: | CGTAAAAACGCAAACTTCATTCTTGACTGCAAAAGCGCCTAAACTTGTAAAGC |
| | L17087.1 | CCGCCCACAAATCTTACATACAGGAGATATTGAGTAATCACTACAAACCCTAT |
| | | AATCGTCAACAATTCAAAACGAATTGCACCCAATAGGATCAGTAAATTCTGCT |
| | | CAAAATATGTAAAAGATACTTTTGCAATTGCTTTATTTTATTATTTGTTACTTTT |
| | | TTTAGTCGTACTTGTCATGCTATTTTTAAGTGGCTTCTCATAAATGAAGGGCTC |
| | | AGAATTGAAGATATAAATACATCAAATAACCCACCTATTTCAAAATTGTTTTAT |
| | | TTTTGTTTATCTCTACAACAAACAACTTTCCTTTATTTTACTACCAACTATTTTC |
| | | CATTCGTTAAAATGAAATTCTCCAAAATTGCCTGTGCTACTGTTTTTGCTTTATC |
| | | TTCTCAAGCTGCCATCATCCACCATGCTCCAGAATTCAACATGAAGAGAGATG |
| | | TTGCTCCAGCTGCCCCAGCTGCTCCAGCTGACCAAGCACCTACTGTTCCTGCAC |
| | | CTCAAGAATTCAATACTGCTATTACCAAAAGAAGTATTATTGGAATTATTATGG |
| | | GTATTCTTGGCAACATTCCACAAGTAATCCAAATCATCATGAGTATTGTCAAAG |
| | | CTTTCAAAGGTAACAAGAGAGAAGATATTGATTCTGTTGTTGCTGGTATCATTG |
| | | CTGATATGCCATTTGTTGTCAGAGCTGTTGACACAGCCATGACTTCTGTTGCTT |
| | | CTACCAAGAGAGATGGAGCTAATGATGACGTTGCTAATGCCGTCGTCAGATTG |
| | | CCAGAAATTGTTGCTCGTGTTGCCACTGGTGTTCAACAATCCATCGAAAATGCC |
| | | AAGAGAGATGGCGTTCCAGATGTTGGCCTTAATCTTGTTGCTAATGCTCCAAGA |
| | | CTTATCTCTAACGTTTTTGATGGCGTCCTGGAAACTGTTCAACAAGCTAAGAGA |
| | | GATGGTCTTGAAGATTTTCTTGATGAACTTCTTCAAAGACTCCCACAACTCATT |
| | | ACTAGATCAGCTGAATCTGCTTTGAAAGACAGTCAACCAGTTAAAAGAGATGC |
| | | CGGCTCAGTAGCACTTAGCAATTTAATCAAAAAGAGCATTGAAACTGTCGGTA |
| | | TTGAAAATGCTGCTCAAATTGTTTCAGAAAGAGATATTTCTTCTTTGATTGAAG |
| | | AATATTTCGGAAAAGCTTAAATGCTCAGCAGATAAAAATTTGTTTTCCACAAG |
| | | CTTAATCTTTTATTCCATAAGTCTTGTGACACTTTTGCTGTAACTGATTTTTTTA |
| | | ATTGTTGTTTTTTGTGGTGGTGTTGAATTGCTTTAATATCATTTTGATAGCATTC |
| | | ACACCATAGTTTATATTACCTATATTTTACTTAGATATCAA |
| SEQ ID NO: | A 20 nt reverse | TTGATATCTAAGTAAAATAT |
| 46 | compliment | |
| | RNA of a | |
| | ECE1 DNA | |
| | sequence | |

### Administering and Treatment

In some aspects, disclosed herein are methods of administering a composition (e.g., pharmaceutical composition) as described herein to a subject who can be a subject in need thereof. In some cases, a method of treating or preventing a disease can comprise administering a composition described herein.

In some aspects, a subject can be a subject in need thereof. In some aspects, a subject can have a disease such as a cancer, a viral disease or a disease affecting the function of an organ such as a heart. In some aspects, a presence of a biomarker can indicate the subject has a cancer, a viral disease, or disease affecting the function of an organ such as a heart. In some aspects, a treatment can at least partially improve or ameliorate a symptom of a disease, as described herein. In some aspects, an amelioration of a disease comprises a reduction in need the size of a tumor, a reduction the number of cancerous cells, a reduction of fatigue, an increase in energy, a reduction in pain, reduced spread of a cancer, increased weight, increased muscle, or any combination thereof. In some aspects, administering compositions as described herein can prevent a disease.

In some aspects, administering can comprise an injection. In some aspects, an injection can comprise an intravenous injection. In some aspects, administering can induce a selective death of cells expressing a biomarker. In some aspects, administering can induce a selective growth inhibition of a cell expressing a biomarker. In some aspects, administering can induce a selective growth of a cell expressing a biomarker. In some aspects, administering can induce a cell surface protein upregulation of a cell expressing a biomarker. In some aspects, administering can induce a secreted chemical or protein from a cell expressing a biomarker. In some aspects, administering can induce these effects in a cell in which a biomarker is not expressed but targeted in other fashions such as by microscopy, by x-ray, by MRI, by PET/CT, by sight, or by loss or gain of a function in a subject such as difficulty breathing, skin change, behavioral issues, cognitive issues, waste secretion and the like.

In some aspects, a pharmaceutical composition can be administered to a subject at a suitable unit dose. The pharmaceutical composition can be in unit dose form. In some cases, unit dose can be meant to refer to pharmaceutical drug products in the form in which they are marketed for use, with a specific mixture of active ingredients and inactive components, diluents, or excipients, in a particular configuration, and apportioned into a particular dose to be delivered. In some instances, unit dose can also sometimes encompass non-reusable packaging, although the FDA distinguishes between unit dose “packaging” or “dispensing”. More than one unit dose can refer to distinct pharmaceutical drug products packaged together, or to a single pharmaceutical drug product containing multiple drugs and/or doses. In some instances, the term unit dose can also sometimes refer to the particles comprising a pharmaceutical composition, and to any mixtures involved. In some cases, types of unit doses may vary with the route of administration for drug delivery, and the substance(s) being delivered. In some aspects, administration can comprise intravenous, intraperitoneal, intra-arterial, intertumoral, subcutaneous, intramuscular, intranasal, topical, oral, or intradermal administration. In some cases, administration can comprise inhalation administration. In some aspects, a dosage regimen can be determined by an attending physician and clinical factors. In some aspects, a dosage for a subject can depend upon many factors, including a subject's size, body surface area, age, sex, general health, a compound to be administered, a time and route of administration, other drugs being administered concurrently, or any combination thereof.

In some aspects, a range of a dose can comprise 0.001 to 1000 μg. In some aspects, a dose can be below or above such a range. In some aspects, a regimen as a regular administration of a pharmaceutical composition can be in a range of 1 μg to 10 mg. In some aspects, a regimen as a regular administration of a pharmaceutical composition can be in a range of 10\hat{ }2 units to 10\hat{ }10 units per day, week or month. In some cases, a unit can be a copy of the engineered polynucleotide, or a vector comprising an engineered polynucleotide. In some aspects, if a regimen comprises a continuous infusion, it can also be in a range of 1 μg to 10,000 mg of pharmaceutical composition or engineered polynucleotide or DNA encoding the engineered polynucleotide or vector containing or encoding the engineered polynucleotide per kilogram of body weight per minute, respectively. In certain instances, the range is from 1 mg per kilogram of body weight to 1000 mg per kilogram of body weight. In some aspects, progress can be monitored by periodic assessment.

In some aspects, a composition described herein can be administered one or more days to a subject in need thereof. In some aspects, administration can occur for about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or about 31 days. In some aspects, administration can occur for about: 1, 2, 3, 4, 5, 6, 7, 8, 9, 10, 11, 12, 13, 14, 15, 16, 17, 18, 19, 20, 21, 22, 23, or about 24 months. In some aspects, administration can occur for about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 or more years. In some cases, administration can occur for life. In some aspects, a pharmaceutical composition described herein can be administered on 2, 3, 4, 5, 6, 7, 8, 9, 10, 11, 12, 13, 14, 15, 16, 17, 18, 19 or 20 or more days. In some cases, a composition described herein can be administered on consecutive days or on nonconsecutive days. In some cases, a composition described herein can be administered to a subject more than one time per day. In some instances, a composition described herein can be administered to a subject: 1, 2, 3, 4, 5, 6, 7, 8, 9, or 10 or more times per day.

In some aspects, disclosed herein are methods of use for compositions as disclosed herein. In some aspects, a daily oral dosage regimen can be from about 0.1 milligram per kilogram (mg/kg) to about 80 mg/kg of total body weight, from about 0.2 mg/kg to about 30 mg/kg, or from about 0.5 mg/kg to about 15 mg/kg. In some aspects, a daily parenteral dosage regimen can comprise from about 0.1 mg/kg to about 10,000 mg/kg of total body weight, from about 0.2 mg/kg to about 5,000 mg/kg, or from about 0.5 mg/kg to about 1,000 mg/kg. In some aspects, a daily topical dosage regimen can be from about 0.1 mg to about 500 mg. In some aspects, a daily inhalation dosage regimen can be from about 0.01 mg/kg to about 1,000 mg/kg per day. In some aspects, an optimal quantity and spacing of individual dosages of a composition can be determined by a nature and extent of a condition being treated, a form, route and site of administration, and a particular subject being treated, and that such optimums can preferably be determined by a method described herein. In some aspects, a number of doses of compositions given per day for a defined number of days, can be ascertained by those skilled in the art using conventional course of treatment determination tests. In some aspects, a dosage regimen can be determined by an attending physician and other clinical factors. In some aspects, dosages for any one subject can depend upon many factors. In some aspects, factors affecting dosage can comprise a subject's size, body surface area, age, a particular compound to be administered, sex, time and route of administration, general health, other drugs being administered concurrently or any combination thereof. In some aspects, progress can be monitored by periodic assessment.

In some cases, a dose regimen can be administered for a duration of about 1 week, about 2 weeks, about 3 weeks, about 4 weeks, about 6 weeks, about 7 weeks, about 8 weeks, about 9 weeks, about 10 weeks, or about 12 weeks. In some cases, a dose regimen can be administered for a duration of about 1 month, about 2 months, about 3 months, about 4 months, about 6 months, about 7 months, about 8 months, about 9 months, about 10 months, or about 12 months. In some cases, a dose regimen can be administered for a duration of about 1 year, about 2 years or more than about 3 years.

In some aspects, disclosed herein are methods of administering an engineered nucleic acid or a DNA encoding an engineered nucleic acid to a subject in combination with another treatment or therapy. In some aspects, a method can further comprise administering one or more additional therapeutics. In some aspects, one or more additional therapeutics can be administered concurrently. In some aspects, one or more additional therapeutics can be administered consecutively. In some aspects, when the additional therapy is a second drug, the second therapy can be included in a pharmaceutical composition in the form of a fixed dose combination drug. In some cases, an additional therapeutic can comprise surgery, chemotherapy, radiation therapy, an anticancer therapy, cryosurgery, thermotherapy, radiochemotherapy, a vaccine, immunotherapy (e.g., an immunotherapeutic agent), hormone therapy, a checkpoint inhibitor, targeted drug therapy, chimeric antigen receptor (CAR) T-cell therapy, a cardiovascular therapy or any combination thereof. In some cases, an additional therapeutic can comprise T-cell transfer therapy.

In certain instances, a co-therapy can include an ace inhibitor such as benazepril, captopril, enalapril maleate, lisinopril, quinapril, ramipril, a salt of these, or any combination thereof. In certain instances, a co-therapy can include an angiotensin 2 receptor antagonist such as candesartan cilexetil, eprosartan mesylate, irbesartan, losartan, telmisartan, valsartan, a salt of these, or any combination thereof. In certain instances, a co-therapy can include an antiarrhythmic such as amiodarone, disopyramide phosphate, dofetilide, flecainide, mexiletine, procainamide, propafenone, quinadine, glucomate, sotalol, tocainide, a salt of these, or any combination thereof. In certain instances, a co-therapy can include an anticoagulant such as dalteparin, enoxaparin, fondaparinux, heparin, warfarin, a salt of these, or any combination thereof. In certain instances, a co-therapy can include a platelet inhibitor such as aspirin, cilostazol, clopidogrel, dipyramidamole, prasugrel, ticlopidine, a salt of these, or any combination thereof. In some instances, a co-therapy can include an antihypertensive such as clonidine, doxazosin mesylate, hydralazine, methyldopa, minoxidil, phenoxybenzamine, phentolamine mesylate, prazosin, terazosin, a salt of these or any combination thereof. In certain instances, a co-therapy can include a beta blocker such as acebutolol, atenolol, betaxolol, bisoprolol, carvedilol, labetalol, metoprolol, nadolol, nebivolol, pindolol, propranolol, sotalol, timolol, a salt of these or any combination thereof. In some instances, a co-therapy can include a calcium channel blocker such as amlodipine besylate, bepridil, diltiazem, felodipine, isradipine, nicardipine, nifedipine, nisoldipine, verapamil, a salt of these or any combination thereof. Other cardiac related co-therapy small molecule drugs can include digitoxin, statins such as atorvastatin, fluvastatin, lovastatin, rosuvastatinm, or nitrates such as nitroglycerin. Salts of these or combinations thereof are also contemplated.

In some cases, an immunotherapeutic agent can comprise a monoclonal antibody or a fragment thereof (e.g. blinatumomab, trastuzumab, alemtuzumab, rituximab, trastuzumab). In some cases, a hormone therapy can comprise abiraterone, anastrozole, exemestane, fulvestrant, letrozole, leuprolide, tamoxifen, a salt of any of these, or any combination thereof. An immunotherapeutic agent can comprise a check-point inhibitor. For example, an inhibitor of an immune checkpoint molecule can be chosen from an inhibitor of one or more of CD155, PD-1, PD-L1, PD-L2, CTLA4, TIM3, LAG3, VISTA, BTLA, TGT, LAIR-1, CD160, 2B4, TGFR beta, KIRs, and CD94/NKG2A.

In some cases, a co-therapy may be administered along with the engineered nucleic acid. In some cases, co-therapy can be a vaccine. A vaccine can comprise a vaccine against a cancer inducing infectious disease, for example a vaccine for human papillomavirus (e.g., recombinant human papillomavirus quadrivalent vaccine) or hepatitis B virus. In some cases, a vaccine can comprise a cancer vaccine (e.g., Talimogene laherparepvec, Sipuleucel-T or a combination thereof). In some cases, an additional therapeutic can comprise, Bacille Calmette-Guérin, imiquimod, IL-2, an interferon (e.g., IFN-alpha, IFN-beta, IFN-gamma), or any combination thereof. In some cases, radiation therapy can comprise external radiation therapy, internal radiation therapy (e.g., brachytherapy), or both. In some instances, radiation therapy can comprise 3D (3-dimensional) conformal radiation therapy (3D-CRT), intensity-modulated radiation therapy (IMRT), image-guided radiation therapy (IGRT) stereotactic body radiation therapy (SBRT), proton therapy, stereotactic ablative radiation therapy (SABR) or any combination thereof. In some aspects, one or more additional therapeutics administered can comprise, a ventilator, an Extracorporeal Membrane Oxygenation (ECMO) machine, nitric oxide, oxygen, saline, interfering RNA therapies, a medicament, a stem cell, or any combination thereof.

In some cases, a small molecule co-therapy be administered with the cyclic nucleic acid of the present disclosure. In certain instances, a co-therapy can include an alkylating agent, a nitrosoureas, an antimetabolite, an anti-tumor antibiotic (e.g., an anthracycline), a topoisomerase inhibitor, a mitotic inhibitor, a corticosteroid, or any combination thereof. In some instances, a chemotherapy can comprise cyclophosphamide, melphalan, temozolomide, 5-fluorouracil, 6-mercaptopurine, cytarabine, gemcitabine, methotrexate, actinomycin-D, bleomycin, daunorubicin, doxorubicin, docetaxel, estramustine, paclitaxel, vinblastine, etoposide, irinotecan, teniposide, topotecan, exatecan, deruxtecan, indenoisoquinoline, a phenanthridine, an indolocarbazole, monomethyl auristatin E, prednisone, methylprednisolone, dexamethasone, a salt of any of these, or a combination of any of these. In certain instances, a co-therapy can include carboplatin, cisplatin, oxaliplatin, lenalidomide, pomalidomide, thalidomide, a salt of any of these, or any combination thereof. In certain instances, a co-therapy can include an anti-viral drug, such as, acyclovir, adefovir, amantadine, amprenavir, umifenovir, atazanavir, baloxavir, bictegravir, boceprevir, bulevirtide, bidofovir, cidofovir, cobicistat, combivir, daclatasvir, darunavir, delavirdine, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, idoxuridine, imiquimod, imunovir, indinavir, lamivudine, letermovir, lopinavir, loviride, maraviroc, methisazone, moroxydine, methisazone, moroxydine, nelfinavir, nevirapine, nexavir, nitazoxanide, norvir, nseltamivir, penciclovir, peramivir, pleconaril, podophyllotoxin, raltegravir, remdesivir, ribavirin, rilpivirine, rimantadine, ritonavir, saquinavir, simeprevir, sofosbuvir, taribavirin, telaprevir, telbivudine, tenofovir, trifluridine, tromantadine, tromantadine, umifenovir, valaciclovir, valganciclovir, vicriviroc, vidarabine, zalcitabine, zanamivir, chloroquine, hydroxychloroquine, ivermectin, a corticosteroid, losartan, vitamin C, sildenafil, a steroid, an anti-inflammatory, a cytokine storm inhibitor, methylprednisolone, a salt of any of these, or any combination thereof. In some aspects, a cytokine storm inhibitor can comprise a chemokine inhibitor, a compound that targets a cholinergic anti-inflammatory pathway, a platelet activating factor (PAF) inhibitor, a resolvin, a lipoxin, a protectin, a COX-2 inhibitor, a compound targeting a chemokine, a compound targeting a T-reg cell, a prostaglandin, a prostaglandin E2 cyclooxygenase inhibitor, or any combination thereof. In some aspects, an anti-inflammatory can comprise aspirin, ibuprofen, naproxen, celecoxib, diclofenac, diflunisal etodolac, famotidine/ibuprofen, flurbiprofen, indomethacin, ketoprofen, mefenamic acid, meloxicam, nabumetone, oxaprozin, piroxicam, sulindac, celecoxib, a salt of any of these, or any combination thereof. In some aspects, a corticosteroid can comprise a glucocorticoid or a mineralocorticoid. In some aspects, a corticosteroid can comprise prednisone, prednisolone, triamcinolone, triamcinolone, methylprednisolone, dexamethasone, cortisol (hydrocortisone), cortisone, prednisone, prednisolone, methylprednisolone, dexamethasone, betamethasone, triamcinolone, fludrocortisone acetate, deoxycorticosterone acetate, corticosterone, aldosterone, deoxycorticosterone, fludrocortisone, prednisolone, a salt of any of these, or any combination thereof.

### Kits

Also described herein are kits comprising compositions and formulations described herein. In some cases, a kit can comprise a container that comprises a composition or formulation. In some instances, a kit can comprise instructions for use. In some instances, a container can be a sterile container. In some cases, a container can be a plastic, a glass, or a metal container.

### Assays

Certain aspects of the disclosure pertain to assays to determine the localization, the concentration, or both the localization and concentration of an engineered nucleic acid (NA) within a nucleolus of a cell, within the nucleus of a cell, within the cytoplasm of a cell, within a bacterium, within cell culture media, within extracellular medium, within a tissue, or within the body of a subject.

Engineered nucleic acids may be engineered to have a reporter signal covalently bonded to the 5′ end of the engineered nucleic acids, the 3′ end of the engineered nucleic acid, or both the 5′ and the 3′ ends. In some instances, the reporter signal is a fluorophore, chromophore, a quantum dot, a metalorganic molecule, enzyme such as but not limited to luciferase or β-galactosidase, a radionucleotide, a fluorescent protein, a reactive die, nucleic acid dye, a cell function dye, and the like.

In some cases, an engineered nucleic acid can be tested in an in vitro fluorescent assay. In some instances, an in vitro assay may include a reporter gene. In some instances, a reporter gene is beta galactosidase, luciferase, a fluorescent protein such as green, red, blue, cyan, or yellow fluorescent protein. In some instances, the reporter gene chloramphenicol acetyltransferase.

In some cases, engineered nucleic acids may be engineered to have binding sites to target the mRNA of a fluorescent reporter gene. In some cases, binding to an mRNA of a fluorescent reporter by an engineered nucleic acid disclosed herein can decrease the amount of signal relative to when an engineered nucleic acid is not present.

In certain instances, an assay to determine the location of an engineered nucleic acid may include but are not limited to fluorescent microscopy, fluorescence in situ hybridization, fluorescence activated cell sorting, and an immunochromatographic assay.

Examples of florescent dyes include: hydroxycoumarin, aminocoumarin, methoxycoumarin, Cascade Blue, pacific blue, pacific orange, lucifer yellow, R-phycoerythrin, PE-Cy5 congugates, PE-Cy7 congugates, red 613, Cy2, Cy3, Cy3B, Cy3.5, and Cy5.5. Examples of nucleic acid dyes include Hoechst 33342, 4′,6-diamidino-2-phenylindole, Hoechst 33258, SYTOX Blue, Chromomycin A3, Mithramycin, YOYO-1, ethidium bromide, acridine orange, STYTOX green, TOTO-1, thiazole orange, CyTRAK orange, propidium iodide, LDS 751, 7-AAD, SYTOX orange, TOTO-3, TO-PRO-3, DRAQ5, DRAQ7, and the like.

Examples of fluorescent proteins include: green fluorescent protein, red fluorescent protein, cyan fluorescent protein, orange fluorescent protein, R-phycoerythrin, phycobilisomes, peridinin chlorophyll, and derivatives thereof.

### Recap

Certain aspects of the disclosure pertain to an engineered nucleic acid, which can alternatively be referred to as an engineered polynucleotide, comprising a plurality of stem loops wherein each stem loop of the plurality is joined to at least one additional stem loop of the plurality of stem loops by a linker sequence, wherein each loop of each stem loop of the plurality of stem loops independently comprises a binding site that is independently configured to hybridize to a target RNA, and wherein the engineered nucleic is not circularized. In some instances, one or more loops of the plurality of stem loops of the engineered polynucleotide can individually contain one, two, three, or more loop structures, at least one of which can be a stem loop, protruding from the one or more loops of the plurality of stem loops of the engineered polynucleotide. In some instances, the one or more loop structures, individually and independently, can comprise from about 5 to about 15 nucleotides.

In certain aspects, the engineered nucleic acid may have from about 50 to about 500 nucleotides. In certain aspects, the engineered nucleic acid may have a plurality of stem loops such as 4, 5, 6, 7, or 8 stem loops. In certain aspects, pertaining to the engineered nucleic acid, each stem loop of the plurality of stem loops individually may include a stem comprising from about 5 to about 20 base pairs or about 12 to about 15 base pairs. In certain aspects regarding the engineered nucleic acid above, the stem can comprise a deletion in one arm of the stem, a mismatch in the stem, or both. In certain aspects regarding the engineered nucleic acid, each stem of each stem loop of the plurality of stem loops may have a substantially complimentary number of nucleotide base pairs. In certain aspects regarding the engineered nucleic acid above, each linker sequence may individually from about 2 to about 12 nucleotides or about 6 to 8 nucleotides in length. Still further, in certain aspects, each loop of each stem loop of the plurality of stem loops individually may include from about 15 to about 75 nucleotides. In certain aspects, each binding site of each stem loop of the plurality of stem loops, when hybridized to the target RNA, individually may include a mismatched base with respect to a corresponding base of the target RNA. In some instances, the engineered nucleic acid is such that each binding site of each stem loop of the plurality of stem loops, when individually hybridized to an individual target RNA, individually comprises a second mismatched base with respect to a second corresponding base of the target RNA. In some instances, the corresponding base of the target RNA is positioned within a first 8 nucleotides of a 5′ end of the target RNA. In certain aspects above regarding the engineered nucleic acid, each stem loop of the plurality of stem loops is the same. In other instances, at least one stem loop of the plurality of stem loops differs from the remaining stem loops of the plurality of stem loops. In certain instances, a binding site of a loop of a stem loop of the engineered nucleic acid is configured upon binding or hybridizing to a target RNA to produce a mismatch between a base of the binding site of the loop of the stem loop and a base of the target RNA. In certain instances, a binding site of a loop of a stem loop of the engineered nucleic acid is configured upon binding or hybridizing to a target RNA to produce a first mismatch between a first base of the binding site of the loop of the stem loop and a first base of the target RNA and a second mismatch between a second base of the binding site of the loop of the stem loop and a second base of the target RNA. In some instances, the engineered polynucleotide when depicted in two dimensions can assume a snowflake like shape (e.g., can be a snowflake). In some instances, the engineered polynucleotides herein can bind or hybridize to target RNAs which may be referred to as sponging the target RNAs. In some instances, the engineered polynucleotide is single stranded in its primary form. In some instances, the engineered polynucleotide can bind or sponge RNA polynucleotides.

In some instances, the mismatch, the second mismatch, or both, is an A/C mismatch where the A can be in the binding site of the loop of the stem loop and the C can be in the target RNA or the A can be in the target RNA and the C can be in the binding site of the loop of the stem loop.

In some instances, the mismatch, the second mismatch, or both, is an A/G mismatch where the A can be in the binding site of the loop of the stem loop and the G can be in the target RNA or the A can be in the target RNA and the G can be in the binding site of the loop of the stem loop.

In some instances, the mismatch, the second mismatch, or both, is a T/G mismatch where the T can be in the binding site of the loop of the stem loop and the G can be in the target RNA or the T can be in the target RNA and the G can be in the binding site of the loop of the stem loop.

In some instances, the mismatch, the second mismatch, or both, is a T/C mismatch where the T can be in the binding site of the loop of the stem loop and the C can be in the target RNA or the T can be in the target RNA and the C can be in the binding site of the loop of the stem loop.

In some instances, the mismatch, the second mismatch, or both, is a U/G mismatch where the U can be in the binding site of the loop of the stem loop and the G can be in the target RNA or the U can be in the target RNA and the G can be in the binding site of the loop of the stem loop and optionally wherein the U/G mismatch can be a wobble base pair.

In some instances, the mismatch, the second mismatch, or both, is a U/C mismatch where the U can be in the binding site of the loop of the stem loop and the C can be in the target RNA or the U can be in the target RNA and the C can be in the binding site of the loop of the stem loop.

In some instances, the loop of a stem loop can comprise a first binding site and a second binding site. In some instances, the first binding site and the second binding site can be the same. In other instances, the first binding site and the second binding site are different.

In some instances, a base of the mismatch can comprise a C, an A, a G, a T, or a U. In some instances, a base of each mismatch can independently comprise a C, an A, a G, a T, or a U. In certain instances, each binding site in each loop of each stem loop of the plurality of stem loops is configured to hybridize to the same target RNA. In other aspects, at least one binding site in a loop of a stem loop of the plurality of stem loops is configured to hybridize to a different target RNA than at least one other binding site of another loop of a stem loop of the plurality of stem loops.

In some aspects, the engineered nucleic acid comprises DNA. In some aspects, the engineered nucleic acid comprises at least about 80%, 85%, 90%, 95%, 96%, 97%, 98%, or 99% sequence identity to any one of SEQ ID NOs: 79-174. In some aspects, the engineered nucleic acid comprises at least about 80%, 85%, 90%, 95%, 96%, 97%, 98%, or 99% sequence identity to any one of SEQ ID NOs: 175-252. In some aspects the engineered nucleic acid comprises RNA. In some aspects, the engineered nucleic acid comprises at least about 80%, 85%, 90%, 95%, 96%, 97%, 98%, or 99% sequence identity to any one of SEQ ID NOs: 253-330. In some aspects, the engineered nucleic acid comprises at least about 80%, 85%, 90%, 95%, 96%, 97%, 98%, or 99% sequence identity to any one of SEQ ID NOs: 47-78. In some aspects, the engineered nucleic acid comprises at least about 80%, 85%, 90%, 95%, 96%, 97%, 98%, or 99% sequence identity to any one of SEQ ID NOs: 331-426.

In some aspects, the engineered nucleic acid substantially displays C4, C5, C6, C7, or C8 stem loop symmetries. In certain aspects, the plurality of stem loops is substantially radially disposed at substantially regular distances around a partial loop comprising the linkers. In certain aspects the nucleic acid comprises a nuclear localization sequence. In certain aspects, the engineered nucleic acid comprises a modified nucleotide. In certain aspects, the modified nucleotide comprises pseudouridine, 5-methylcytidine, n1-methylpseudouridine, N6-methyladenosine, or any combination thereof.

In certain aspects, the engineered nucleic acid is RNA. In other aspects, the nucleic acid is DNA. In certain aspects disclosed herein is a DNA encoding an RNA engineered nucleic acid.

In certain alternative aspects, the engineered nucleic acid comprises a chemical modification. In some instances, the chemical modification may include a phosphodiester modification, methylation of a hydroxyl group, the presence of an epigenetically marked base, a deoxyribose sugar, or a combination thereof.

Certain further aspects of the disclosure relate to a vector containing or encoding an engineered nucleic acid. In certain instances, the vector is a nanoparticle vector. In certain instances, the vector is a liposomal vector. In certain instances, the vector is a viral vector. Still further, the viral vector may be a DNA viral vector. In certain instances, the viral vector is an adeno associated viral vector. In certain instances, the viral vector is an adenoviral vector. In certain instances, the viral vector is a retroviral vector. In certain instances, the vector may include a nuclear localization sequence. In the case of the vector being a liposomal vector, the liposomal vector may include RNA. In other instances, the liposomal vector may include DNA. In certain instances, wherein the liposomal vector includes DNA, the liposomal vector may comprise a plasmid.

Other aspects of the disclosure pertain to a pharmaceutical composition of the engineered nucleic acid or a DNA encoding an engineered nucleic acid or a vector containing the engineered nucleic acid or a nucleic acid encoding the engineered nucleic acid with a pharmaceutically acceptable: excipient, diluent, or carrier. In certain aspects regarding an excipient, the excipient may include a buffering agent, a stabilizer, an antioxidant, a binder, a diluent, a dispersing agent, a rate controlling agent, a lubricant, a glidant, a disintegrant, a plasticizer, a preservative, or any combinations thereof. In certain aspects regarding a diluent, the diluent may include distilled water, physiological saline, Ringer's solutions, dextrose solution, a cell growth medium, phosphate buffered saline (PBS), or any combination thereof. In certain aspects regarding a pharmaceutical composition, the pharmaceutical composition may additionally include a solubilizing agent or a combination thereof. In certain aspects, the pharmaceutical composition may be in unit dose form.

Other aspects of the disclosure pertain to a kit including the engineered nucleic, a DNA encoding the engineered nucleic acid, a vector, or a pharmaceutical composition of the engineered nucleic acid, or a combination thereof and a container.

Other aspects of the disclosure include a method of treating or preventing a disease or condition in a subject, that can be a human, that can be in need thereof, the method comprising administering to the subject a therapeutically effective amount of the engineered nucleic acid, a DNA encoding an engineered nucleic acid RNA, a vector, or a pharmaceutical composition, thereby treating or preventing the disease or condition. In certain instances, the administering is in an amount of from about 0.001 mg to about 10,000 mg of the engineered nucleic acid, the DNA encoding a nucleic acid, the vector, or the pharmaceutical composition per kg of body weight of the subject. In certain instances, the administering is performed at least once in a 24-hour time period. In other instances, the administering is performed 2, 3, 4, 5, 6, 7, 8, 9, 10, 11, 12, 13, 14, 15, 16, 17, 18, 19, 20, 21, 22, 23, or 24 times in a 24-hour time period. In certain other instances, the administering is performed one or more times per week, one or more times per month, or one or more times per year. In certain instances, the administering is intramuscular, intravenous, intraocular, intraperitoneal, intracardial, subcutaneous, intracranial, intrathecal, oral, inhalation, or any combination thereof.

In certain aspects, the disease or condition comprises a cardiac disease, a neurological disease, cancer, a fungal disease or a viral disease.

In instances wherein the disease or condition comprises a cardiac disease, the disease may include hypertension, a metabolic syndrome, a valve disease, cardiac hypertrophy, cardiac hypotrophy, cardiac fibrotic remodeling, cardiac wall stiffness, stable angina, unstable angina, variant angina, atrial fibrillation, hart block, premature atrial complex, atrial flutter, paroxysmal supraventricular tachycardia, Wolff-Parkinson-White syndrome, premature ventricular complex, ventricular tachycardia, ventricular fibrillation, long QT syndrome, dilated cardiomyopathy, hypertrophic cardiomyopathy, restrictive cardiomyopathy, congestive heart failure, arterial septal defect, ventricular septal defect, patent ductus arteriosis, pulmonic stenosis, congenital aortic stenosis, coarctation of aorta, tetraology of Fallot, tricuspid atresia, truncus arteriosus, Ebstein's anomaly of the tricuspid valve, cor pulmonale, myocardial infarction, mitral stenosis, mitral valve regurgitation, mitral valve prolapse, aortic stenosis, aortic regurgitation, tricuspid stenosis, tricuspid regurgitation, myocarditis, pericarditis, rheumatic heart disease, cardiac tumor, aortic aneurysm, arteriosclerosis, atherosclerosis, aortic dissection, hypertension, transient ischemic attack, other cardiac related diseases, or a combination thereof.

In some instances, a target RNA can comprise between about 5 and about 500 nucleobases. In some instances, the target RNA can be messenger RNA (mRNA), ribosomal RNA (rRNA), transfer RNA (tRNA), ribozymes (RNA enzymes), transfer messenger RNA (tmRNA), double stranded RNA (dsRNA), small nuclear RNA (ssRNA), small nucleolar RNA (snoRNA), Piwi-interacting RNAs (piRNA), long non-coding RNA (lncRNA), or any combination thereof.

In certain instances wherein the disease or condition comprises a cancer, the cancer may include at least one of: a melanoma, a hepatocellular carcinoma, a breast cancer, a lung cancer, a non-small lung cancer, a peritoneal cancer, a prostate cancer, a bladder cancer, an ovarian cancer, a leukemia, a lymphoma, a renal cell carcinoma, a pancreatic cancer, an epithelial carcinoma, a gastric/GE junction adenocarcinoma, a cervical cancer, a colon carcinoma, a colorectal cancer, a duodenal cancer, a pancreatic adenocarcinoma, an adenoid cystic, a sarcoma, a mesothelioma, a glioblastoma multiforme, a astrocytoma, a multiple myeloma, a prostate carcinoma, a hepatocellular carcinoma, a cholangiocarcinoma, a pancreatic adenocarcinoma, a head and neck squamous cell carcinoma, a cervical squamous-cell carcinoma, an osteosarcoma, an epithelial ovarian carcinoma, an acute lymphoblastic lymphoma, a myeloproliferative neoplasm, any other malignant condition or any variant thereof, or any combination thereof.

In instances wherein the disease or condition comprises a viral disease, the viral disease may include a coronavirus disease, such as COVID caused by a SARS-COV-2 and variants thereof, an influenza disease, a parainfluenza virus disease, a herpesvirus disease, an adenovirus disease, a flavivirus disease, a retroviral disease, a paramyxovirus-based disease, a parvovirus-based disease, or any combination thereof.

In certain instances, regarding a condition or disease, the treatment may further include concurrently or consecutively administering a second therapy.

In certain instances, for example when the disease or condition is the cardiac disease or condition, the second therapy may include an ace inhibitor, an angiotensin 2 receptor antagonist, an antiarrhythmic, an anticoagulant, a platelet inhibitor, an antihypertensive, a beta blocker, a calcium channel blocker, digitoxin, a statin, nitroglycerin, or a combination thereof.

In certain instances, regarding a viral disease, the second therapy may include an antiviral drug, an anti-inflammatory, a cytokine storm inhibitor, or any combination thereof.

In certain instances of administering a pharmaceutical composition, the pharmaceutical composition may be in a liquid dosage that is administered at a volume of about 1 ml to about 5 ml, about 5 ml to about 10 ml, about 15 ml to about 20 ml, about 25 ml to about 30 ml, about 30 ml to about 50 ml, about 50 ml to about 100 ml, about 100 ml to about 150 ml, about 150 ml to about 200 ml, about 200 ml to about 250 ml, about 250 ml to about 300 ml, about 300 ml to about 350 ml, about 350 ml to about 400 ml, about 400 ml to about 450 ml, about 450 ml to about 500 ml, about 500 ml to about 750 ml, or about 750 ml to about 1000 ml. In certain instances, the subject receives the pharmaceutical composition at a dosing level from about 0.001 mg/kg to about 1000 mg/kg, wherein mg/kg is mg of the engineered polynucleotide, a DNA encoding the engineered polynucleotide, or a vector containing or encoding the engineered polynucleotide per kilogram of subject body weight. In certain instances, the dosage is in a liquid dosage form, a solid dosage form, an inhalable dosage form, an intranasal dosage form, a liposomal formulation, or any combinations thereof. In certain instances, the administration comprises at least partially systemic administration. In certain instances, the at least partially systemic administration may include at least one of: an oral administration, an intravenous administration, an intranasal administration, a sublingual administration, a rectal administration, a transdermal administration, an intradermal administration, an intraurethral administration, an intravaginal administration, an intrathecal administration, an intramuscular administration, an intraperitoneal administration, an intratumoral administration, or any combinations thereof.

Other aspects of the disclosure include the engineered nucleic acid above, the DNA encoding a nucleic acid, or the vector. In some instances, the engineered polynucleotide can be a ribonucleic acid (RNA). In some instances, the engineered polynucleotide can be a deoxyribonucleic acid (DNA).

Certain aspects of the disclosure may include an engineered polynucleotide, which can be an RNA, wherein a binding site of a loop of a stem loop that is proximal to the 5′ end of the engineered polynucleotide binds or hybridizes to a binding site of a loop of a stem loop that is proximal to the 3′ end of the engineered polynucleotide.

Other aspects of the disclosure pertain to an in vitro assay, the assay comprising conducting the in vitro assay employing the engineered nucleic acid above, the DNA encoding a nucleic acid, or the vector containing or encoding the engineered nucleic acid. In certain aspects of the in vitro assay, the assay comprises detection of a reporter gene. In certain aspects the reporter gene is luciferase, beta galactosidase, a fluorescent protein, chloramphenicol acetyltransferase, or any combination thereof.

Certain other aspects of the disclosure include decreasing expression of a polypeptide translated from an mRNA template in a cell by employing the engineered polynucleotide, a polynucleotide encoding the engineered polynucleotide, or a vector containing or encoding the engineered polynucleotide to diminish directly or indirectly availability of the mRNA template for translation to a polypeptide when compared to an otherwise comparable cell that does not contain the engineered polynucleotide, a polynucleotide encoding the engineered polynucleotide, or a vector containing or encoding the engineered polynucleotide. The decreasing can be determined in an in vitro assay.

Certain other aspects of the disclosure concern a method of sequestering a plurality of target RNAs with an engineered nucleic acid comprising binding at least three loops of three stem loops of the plurality of stem loops of the engineered nucleic acid to one or more target RNAs which encode proteins. The target RNAs can be in a bacterium. In certain instances, the target RNAs can code for two or more proteins in a metabolic pathway is found in bacteria. Still further, the metabolic pathway comprises using an engineered nucleic acid to bind or hybridize a target RNA encoding a protein comprising PBP1a/b, PBP4, PBP2c, PBP2d, PBP2a, PbpH, PBP2b, PBP3, SpoVD, PBP4b, PBP5, PBP4a, DacF, PbpX, MepA, AmpC, AmpH or any combination thereof. Still further, in certain instances, the metabolic pathway comprises affecting the shape of a bacteria and the a target RNA encoding the proteins comprise PBP4, PBP5, PBP7, AmpC, AmpH, or any combination thereof

### EXAMPLES

### Example 1: Generating “Snowflake” Engineered Nucleic Acids

A stem loop or hairpin loop structure was engineered in which the loop was formed by one miR-155-5p binding site, i.e., a binding sequence representing the reverse complement (antisense sequence) of miR-155-5p or a variant thereof harboring a single mutation forming a mismatch in the microRNA seed region upon miRNA binding, and in which the stem was formed by 5′ and 3′ adjacent complementary sequences. Auxiliary arm sequences were inserted in between the miR-155-5p binding site and each stem-forming extension.

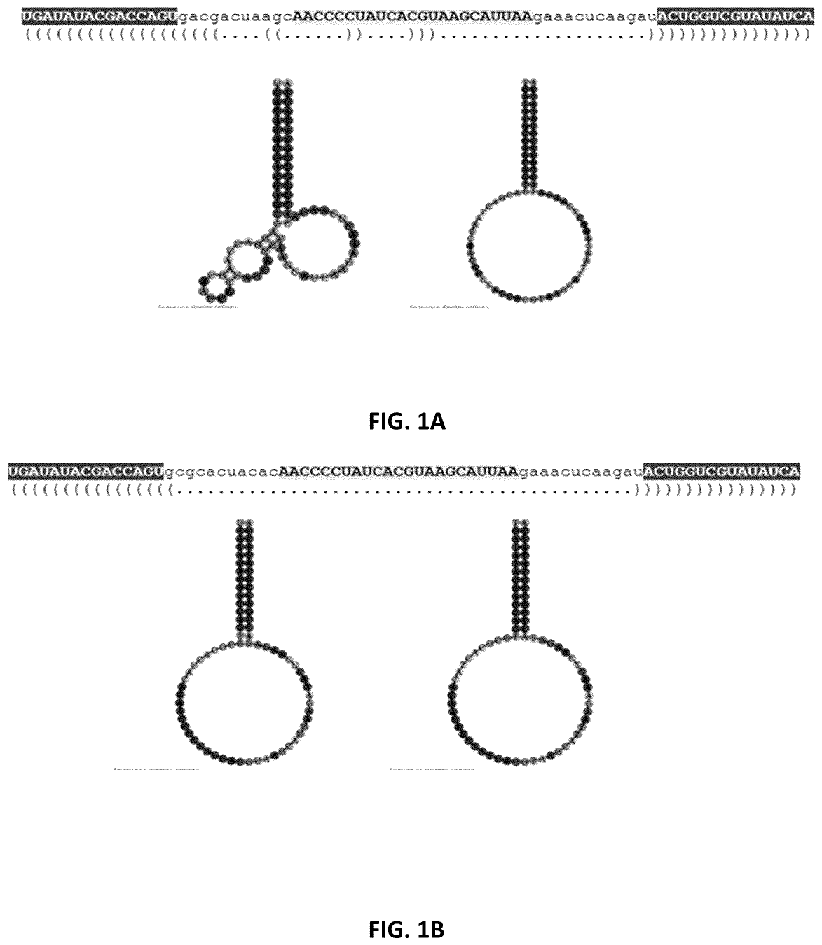

The auxiliary arm sequences were selected to neither form any secondary structure within themselves, nor with each other, nor with the miR-155-5p binding site but instead to support the formation of a large unpaired loop comprising the miR-155-5p binding site and the auxiliary arm sequences (FIG. 1A (SEQ ID NO: 47) and FIG. 1B (SEQ ID NO: 48) found in Table 5 which shows a nested stem loop comprising two regions within a loop between the binding sequence of the loop. The sequence lengths were as follows: 15 nt 5′ and 3′ stem forming sequences, 11 nt 5′ auxiliary arm region, 12 nt 3′ auxiliary arm region.

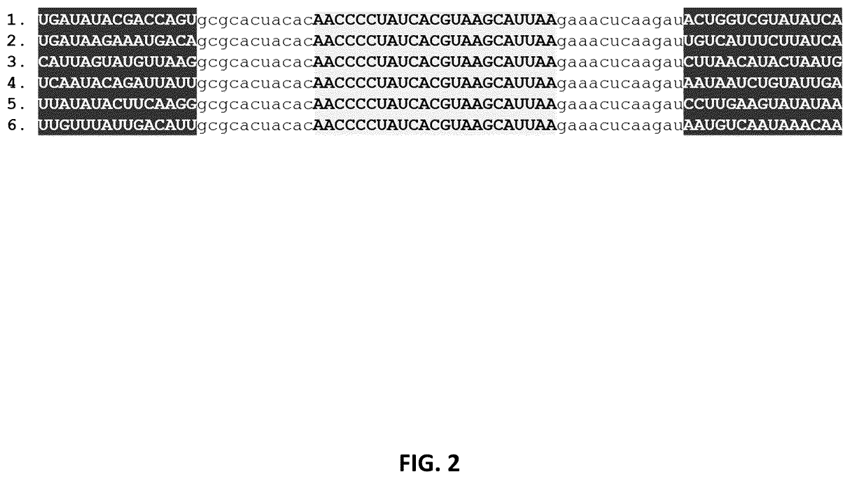

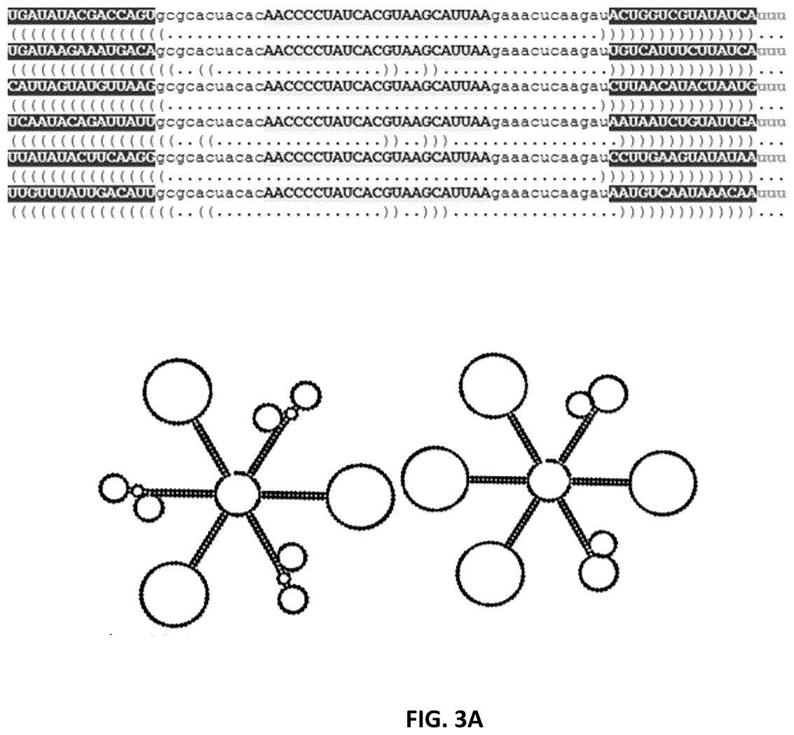

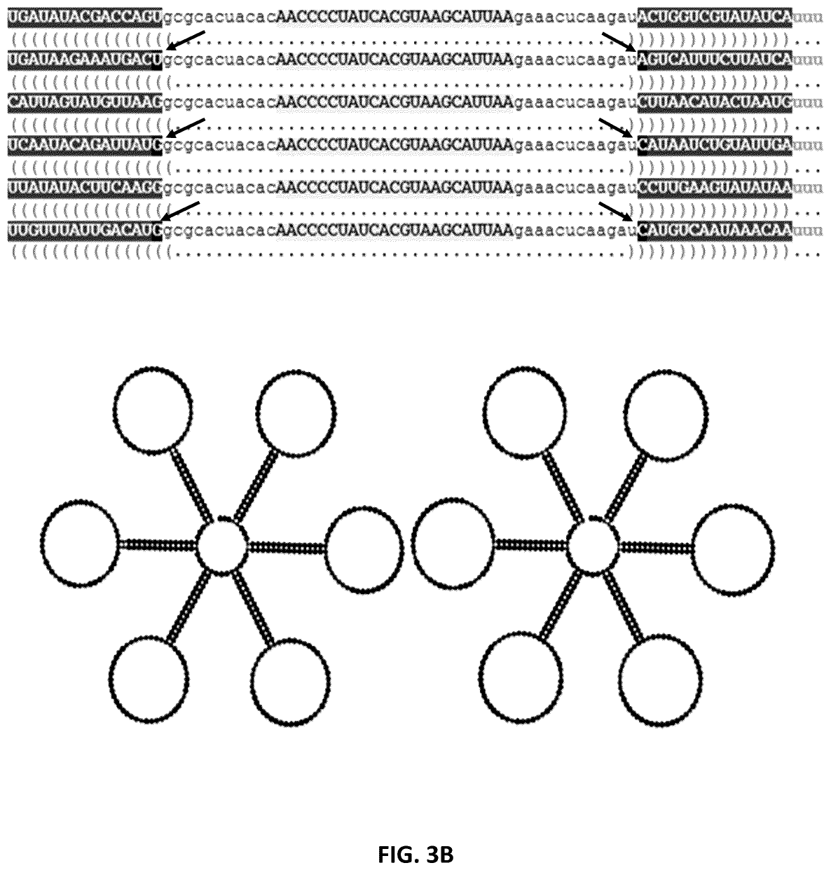

3 nt linkers were used to join six individual stem-loops in a flexible way (FIG. 2) together to form a ‘snowflake’-like structure (FIG. 3A) See Table 5. uuu sequences were chosen as linkers to avoid base pairing within linkers. Incorporation of linkers resulted in pairing within some loops in the resulting structure (FIG. 3A) (SEQ ID NOs: 49-54) See Table 5. Thus, stem sequences were further edited manually to eliminate pairing within loops (FIG. 3B) (SEQ ID NOs: 55-60). See Table 5.

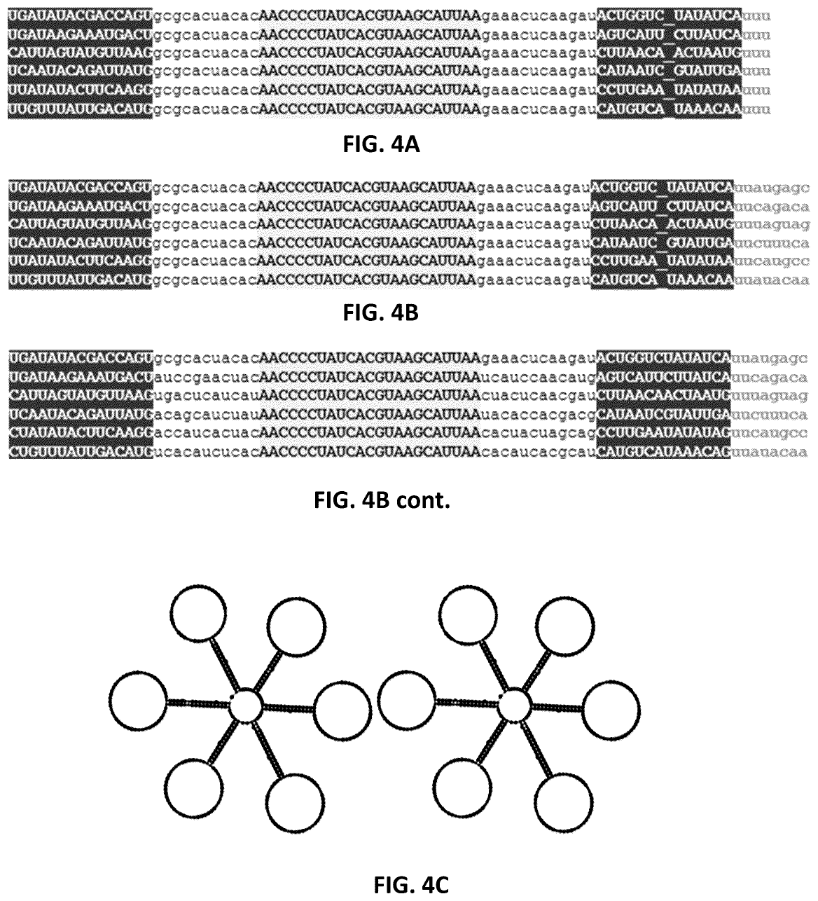

Perfectly base-paired double-stranded RNA can give rise to A-to-I editing in the nucleus and can trigger RNA interference (RNAi) in the cytoplasm. To improve the flexibility of the stem and to suppress editing and/or RNAi, a single base deletion was introduced at the center of the stem (FIG. 4A) (SEQ ID NOs: 61-66). See Table 5. Alternatively, a mismatch or larger internal loops, preferably symmetric internal loops, can be introduced instead. Structures with a mismatch and larger internal loops (symmetric loops) can be further tested. Additionally, different linker sequences that were longer (8 nt) with approximately 30% GC content and preferably more Us were also used to separate each individual stem-loop (FIG. 4B) (SEQ. ID. Nos 67-72). See Table 5. Lastly, to reduce the number of repeats, that can result in frequent recombination events, within the RNA structure, different 5′ and 3′ auxiliary arm sequences, while maintaining the length, were generated for each stem-loop (FIG. 4B cont.) (SEQ ID NOs: 73-78). See Table 5. Table 5 provides engineered nucleic acid design sequences (e.g., an auxiliary arm and binding site sequence) and descriptions of the sequences.

TABLE 5
Engineered nucleic acid design sequences

| SEQ ID and Description | Sequence |
|---|---|
| SEQ ID NO: 47 | UGAUAUACGACCAGUgacgacuaagcAACCCCUAUCACGU |
| Auxiliary arm and miR- | AAGCAUUAAgaaacucaagauACUGGUCGUAUAUCA |
| 155-5p binding sites paired | |
| SEQ ID NO: 48 | UGAUAUACGACCAGUgcgcacuacacAACCCCUAUCACGU |
| Auxiliary arm and miR- | AAGCAUUAAgaaacucaagauACUGGUCGUAUAUC |
| 155-5p binding sites | |
| unpaired | |
| SEQ ID NO: 49 | UGAUAUACGACCAGUgcgcacuacacAACCCCUAUCACGU |
| Auxiliary arm and miR- | AAGCAUUAAgaaacucaagauACUGGUCGUAUAUCAuuu |
| 155-5p binding sites paired | |
| alternate 1 | |
| SEQ ID NO: 50 | UGAUAAGAAAUGACAgcgcacuacacAACCCCUAUCACGU |
| Auxiliary arm and miR- | AAGCAUUAAgaaacucaagauUGUCAUUUCUUAUCAuuu |
| 155-5p binding sites paired | |
| alternate 2 | |
| SEQ ID NO: 51 | CAUUAGUAUGUUAAGgcgcacuacacAACCCCUAUCACGU |
| Auxiliary arm and miR- | AAGCAUUAAgaaacucaagauCUUAACAUACUAAUGuuu |
| 155-5p binding sites paired | |
| alternate 3 | |
| SEQ ID NO: 52 | UCAAUACAGAUUAUUgcgcacuacacAACCCCUAUCACGU |
| Auxiliary arm and miR- | AAGCAUUAAgaaacucaagauAAUAAUCUGUAUUGAuuu |
| 155-5p binding sites paired | |
| alternate 4 | |
| SEQ ID NO: 53 | UUAUAUACUUCAAGGgcgcacuacacAACCCCUAUCACGU |
| Auxiliary arm and miR- | AAGCAUUAAgaaacucaagauCCUUGAAGUAUAUAAuuu |
| 155-5p binding sites paired | |
| alternate 5 | |
| SEQ ID NO: 54 | UUGUUUAUUGACAUUgcgcacuacacAACCCCUAUCACGU |
| Auxiliary arm and miR- | AAGCAUUAAgaaacucaagauAAUGUCAAUAAACAAuuu |
| 155-5p binding sites paired | |
| alternate 6 | |
| SEQ ID NO: 55 | UGAUAUACGACCAGUgcgcacuacacAACCCCUAUCACGU |
| Auxiliary arm and miR- | AAGCAUUAAgaaacucaagauACUGGUCGUAUAUCAuuu |
| 155-5p binding sites | |
| unpaired alternate 1 | |
| SEQ ID NO: 56 | UGAUAAGAAAUGACUgcgcacuacacAACCCCUAUCACGU |
| Auxiliary arm and miR- | AAGCAUUAAgaaacucaagauAGUCAUUUCUUAUCAuuu |
| 155-5p binding sites | |
| unpaired alternate 2 | |
| SEQ ID NO: 57 | CAUUAGUAUGUUAAGgcgcacuacacAACCCCUAUCACGU |
| Auxiliary arm and miR- | AAGCAUUAAgaaacucaagauCUUAACAUACUAAUGuuu |
| 155-5p binding sites | |
| unpaired alternate 3 | |
| SEQ ID NO: 58 | UCAAUACAGAUUAUGgcgcacuacacAACCCCUAUCACGU |
| Auxiliary arm and miR- | AAGCAUUAAgaaacucaagauCAUAAUCUGUAUUGAuuu |
| 155-5p binding sites | |
| unpaired alternate 4 | |
| SEQ ID NO: 59 | UUAUAUACUUCAAGGgcgcacuacacAACCCCUAUCACGU |
| Auxiliary arm and miR- | AAGCAUUAAgaaacucaagauCCUUGAAGUAUAUAAuuu |
| 155-5p binding sites | |
| unpaired alternate 5 | |
| SEQ ID NO: 60 | UUGUUUAUUGACAUGgcgcacuacacAACCCCUAUCACGU |
| Auxiliary arm and miR- | AAGCAUUAAgaaacucaagauCAUGUCAAUAAACAAuuu |
| 155-5p binding sites | |
| unpaired alternate 6 | |
| SEQ ID NO: 61 | UGAUAUACGACCAGUgcgcacuacacAACCCCUAUCACGU |
| Auxiliary arm and miR- | AAGCAUUAAgaaacucaagauACUGGUC_UAUAUCAuuu |
| 155-5p binding sites single | |
| base deletion alternate 1 | |
| SEQ ID NO: 62 | UGAUAAGAAAUGACUgcgcacuacacAACCCCUAUCACGU |
| Auxiliary arm and miR- | AAGCAUUAAgaaacucaagauAGUCAUU_CUUAUCAuuu |
| 155-5p binding sites single | |
| base deletion alternate 2 | |
| SEQ ID NO: 63 | CAUUAGUAUGUUAAGgcgcacuacacAACCCCUAUCACGU |
| Auxiliary arm and miR- | AAGCAUUAAgaaacucaagauCUUAACA ACUAAUGuuu |
| 155-5p binding sites single | |
| base deletion alternate 3 | |
| SEQ ID NO: 64 | UCAAUACAGAUUAUGgcgcacuacacAACCCCUAUCACGU |
| Auxiliary arm and miR- | AAGCAUUAAgaaacucaagauCAUAAUC_GUAUUGAuuu |
| 155-5p binding sites single | |
| base deletion alternate 4 | |
| SEQ ID NO: 65 | UUAUAUACUUCAAGGgcgcacuacacAACCCCUAUCACGU |
| Auxiliary arm and miR- | AAGCAUUAAgaaacucaagauCCUUGAA_UAUAUAAuuu |
| 155-5p binding sites single | |
| base deletion alternate 5 | |
| SEQ ID NO: 66 | UUGUUUAUUGACAUGgcgcacuacacAACCCCUAUCACGU |
| Auxiliary arm and miR- | AAGCAUUAAgaaacucaagauCAUGUCA_UAAACAAuuu |
| 155-5p binding sites single | |
| base deletion alternate 6 | |
| SEQ ID NO: 67 | UGAUAUACGACCAGUgcgcacuacacAACCCCUAUCACGU |
| Auxiliary arm and miR- | AAGCAUUAAgaaacucaagauACUGGUC_UAUAUCAuuaugag |
| 155-5p binding sites single | c |
| base deletion alternate 1 | |
| with 8 nt linker | |
| SEQ ID NO: 68 | UGAUAAGAAAUGACUgcgcacuacacAACCCCUAUCACGU |
| Auxiliary arm and miR- | AAGCAUUAAgaaacucaagauAGUCAUU_CUUAUCAuucagac |
| 155-5p binding sites single | a |
| base deletion alternate 2 | |
| with 8 nt linker | |
| SEQ ID NO: 69 | CAUUAGUAUGUUAAGgcgcacuacacAACCCCUAUCACGU |
| Auxiliary arm and miR- | AAGCAUUAAgaaacucaagauCUUAACA_ACUAAUGuuuagua |
| 155-5p binding sites single | g |
| base deletion alternate 3 | |
| with 8 nt linker | |
| SEQ ID NO: 70 | UCAAUACAGAUUAUGgcgcacuacacAACCCCUAUCACGU |
| Auxiliary arm and miR- | AAGCAUUAAgaaacucaagauCAUAAUC_GUAUUGAuucuuuc |
| 155-5p binding sites single | a |
| base deletion alternate 4 | |
| with 8 nt linker | |
| SEQ ID NO: 71 | UUAUAUACUUCAAGGgcgcacuacacAACCCCUAUCACGU |
| Auxiliary arm and miR- | AAGCAUUAAgaaacucaagauCCUUGAA_UAUAUAAuucaugc |
| 155-5p binding sites single | c |
| base deletion alternate 5 | |
| with 8 nt linker | |
| SEQ ID NO: 72 | UUGUUUAUUGACAUGgcgcacuacacAACCCCUAUCACGU |
| Auxiliary arm and miR- | AAGCAUUAAgaaacucaagauCAUGUCA_UAAACAAuuauaca |
| 155-5p binding sites single | a |
| base deletion alternate 6 | |
| with 8 nt linker | |
| SEQ ID NO: 73 | UGAUAUACGACCAGUgcgcacuacacAACCCCUAUCACGU |
| Auxiliary arm and miR- | AAGCAUUAAgaaacucaagauACUGGUCUAUAUCAuuaugagc |
| 155-5p binding sites no | |
| deletion alternate 1 with 8 | |
| nt linker | |
| SEQ ID NO: 74 | UGAUAAGAAAUGACUauccgaacuacAACCCCUAUCACGU |
| Auxiliary arm and miR- | AAGCAUUAAucauccaacaugAGUCAUUCUUAUCAuucagaca |
| 155-5p binding sites no | |
| deletion alternate 2 with 8 | |
| nt linker | |
| SEQ ID NO: 75 | CAUUAGUAUGUUAAGugacucaucauAACCCCUAUCACGU |
| Auxiliary arm and miR- | AAGCAUUAAcuacucaacgauCUUAACAACUAAUGuuuaguag |
| 155-5p binding sites no | |
| deletion alternate 3 with 8 | |
| nt linker | |
| SEQ ID NO: 76 | UCAAUACAGAUUAUGacagcaucuauAACCCCUAUCACGU |
| Auxiliary arm and miR- | AAGCAUUAAuacaccacgacgCAUAAUCGUAUUGAuucuuuca |
| 155-5p binding sites no | |
| deletion alternate 4 with 8 | |
| nt linker | |
| SEQ ID NO: 77 | CUAUAUACUUCAAGGaccaucacuacAACCCCUAUCACGU |
| Auxiliary arm and miR- | AAGCAUUAAcacuacuagcagCCUUGAAUAUAUAGuucaugcc |
| 155-5p binding sites no | |
| deletion alternate 5 with 8 | |
| nt linker | |
| SEQ ID NO: 78 | CUGUUUAUUGACAUGucacaucucacAACCCCUAUCACGU |
| Auxiliary arm and miR- | AAGCAUUAAcacaucacgcauCAUGUCAUAAACAGuuauacaa |
| 155-5p binding sites no | |
| deletion alternate 6 with 8 | |
| nt linker | |
| SEQ ID NO: 427 | UGAUAUACGACCAGUgcgcacuacacAACCCCUAUCACGU |
| Auxiliary arm and miR- | AAGCAUUAAgaaacucaagauACUGGUCGUAUAUCA |
| 155-5p binding sites paired | |
| alternate 1 | |
| SEQ ID NO: 428 | UGAUAAGAAAUGACAgcgcacuacacAACCCCUAUCACGU |
| Auxiliary arm and miR- | AAGCAUUAAgaaacucaagauUGUCAUUUCUUAUCA |
| 155-5p binding sites paired | |
| alternate 2 | |
| SEQ ID NO: 429 | CAUUAGUAUGUUAAGgcgcacuacacAACCCCUAUCACGU |
| Auxiliary arm and miR- | AAGCAUUAAgaaacucaagauCUUAACAUACUAAUG |
| 155-5p binding sites paired | |
| alternate 3 | |
| SEQ ID NO: 430 | UCAAUACAGAUUAUUgcgcacuacacAACCCCUAUCACGU |
| Auxiliary arm and miR- | AAGCAUUAAgaaacucaagauAAUAAUCUGUAUUGA |
| 155-5p binding sites paired | |
| alternate 4 | |
| SEQ ID NO: 431 | UUAUAUACUUCAAGGgcgcacuacacAACCCCUAUCACGU |
| Auxiliary arm and miR- | AAGCAUUAAgaaacucaagauCCUUGAAGUAUAUAA |
| 155-5p binding sites paired | |
| alternate 5 | |
| SEQ ID NO: 432 | UUGUUUAUUGACAUUgcgcacuacacAACCCCUAUCACGU |
| Auxiliary arm and miR- | AAGCAUUAAgaaacucaagauAAUGUCAAUAAACAA |
| 155-5p binding sites paired | |
| alternate 6 | |

### Example 2: snoRNA and piRNA Targeting

### Background

Based on inferred biology and molecular mechanisms, it is hypothesized that engineered nucleic acids could be customized to target and sequester (or sponge) other small non-coding nucleic acids such as RNAs that are not microRNAs. These include but are not limited to small nucleolar RNAs (snoRNAs) and piwi-interacting RNAs (piRNAs).

SnoRNAs are non-coding RNAs of 60-300 nucleotides (nt) long, derived largely from gene introns. They play roles that include rRNA processing, regulation of mRNA splicing and editing, during cellular stress response and metabolic homeostasis. These functions may be implicated in the initiation and progression of pathology. In terms of disease landscape, their role and molecular mechanisms have been widely studied in cancer, neurodegenerative disorders and viral diseases.

Prominent examples are v-snoRNA1 (viral small nucleolar RNA1) which was identified in B lymphocytes infected with Epstein-Barr virus (EBV), which regulates virus life cycle by interacting with BALF5, a viral DNA polymerase mRNA, inducing its hydrolysis. SnoRNAs are also suggested to sustain drug-resistance in cancer cells in which targeted regulation of snoRNA expression increase their sensitivity to tamoxifen therapy. Three box C/D snoRNAS U32a, U33 and U35a are can be relevant in lipotoxic and oxidative stress where the loss of these RNAs prevented propagation of oxidative stress in a well-established lipopolysaccharide (LPS)-mediated liver injury model in mice.

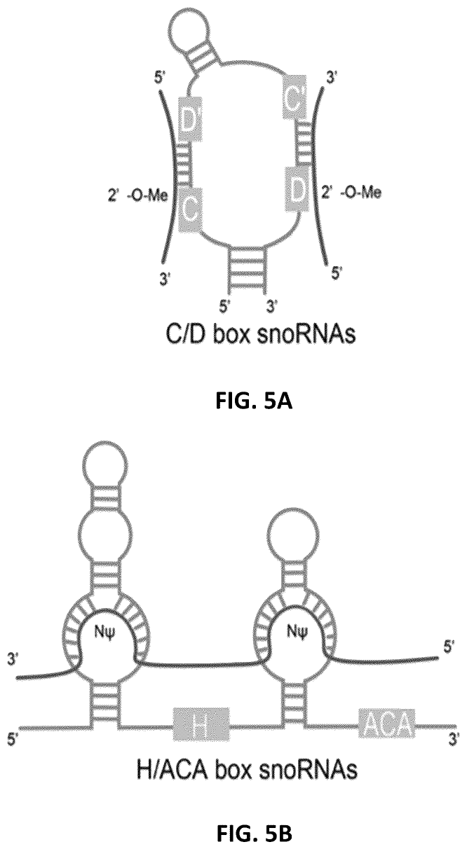

H/ACA box snoRNAs have a characteristic “hairpin-hinge-hairpin-tail” structure. H/ACA box snoRNAs also carry two conserved sequence elements: box H, ANANNA sequence (N represents any nucleotide), and box ACA, trinucleotide ACA as seen in FIG. 5A and FIG. 5B. An internal bulged loop is located in the hairpin of H/ACA box snoRNAs with a 9-13 nt sequence on each strand complementary to substrate RNAs. By designing antisense sites to substrate RNA target sites, engineered nucleic acids can be able to sponge snoRNAs.

In the past, antisense oligonucleotides ASOs have been designed and tested against inhibition of snoRNAs such as U84, HBII295 box C/D snoRNA and H/ACA38 snoRNA6. From assessing target effectiveness to different regions within snoRNA, the study showed that an antisense design against the guide sequence upstream of D box for C/D snoRNAs or against the guide sequence within the internal bulged loop of H/ACA box snoRNAs was most effective.

ASOs delivered to target snoRNAs result in the formation of a snoRNA-ASO hybrid that can be recognized and cleaved by an endogenous endonuclease, likely RNase H. siRNAs are unable to antagonize snoRNA function as they require RISC processing in cytoplasm prior to snoRNA inhibition. Engineered sponges that form snoRNA-RNA hybrid could potentially be degraded by RNase III-like enzymes. Alternatively, single stranded engineered DNA can be designed to carry sponge sites that would lead to snoRNA-DNA hybrid and thus RNase H degradation.

### Methods

Engineered nucleic acids will be synthesized to sequester snoRNAs through antisense interaction with specific motifs that otherwise interact with substrate RNAs. The knockdown specificity of engineered nucleic acids will be assessed by the following ways.

Cells are transfected with engineered nucleic acid sponges carrying binding sites for target ncRNAs. Total RNA is hybridized to an A488 antisense RNA probe against target snoRNAs synthesized using T7 RNA polymerase. RNA fragments are separated by denaturing urea polyacrylamide gel electrophoresis and fluorescent bands are visualized. The intensity of the fluorescent bands corresponds to the levels of respective snoRNAs and would demonstrate the efficacy of engineered nucleic acid sponges. To confirm direct RNA-RNA interaction between engineered nucleic acid sponges and target ncRNA, a biotinylated antisense RNA probe is employed with streptavidin beads to perform an RNA pull-down assay.

Functional snoRNA knockdown are validated by detecting the loss of ribose 2′-0 methylation or pseudouridylation activity. Expression plasmids are constructed with snoRNA target sequences (complementary to the guide sequence) inserted between human RNA polymerase I promoter and the terminator. Plasmids are transfected into cells and the methylation or psuedouridiylation state of the expressed pol I transcripts are monitored by a primer extension system using AMV reverse transcriptase. At low dNTP concentrations, RNA modifications sterically interfere with the passage of reverse transcriptase which results in cDNA termination 1 nt before or at the modified nucleotide. The size of primer extension products can be analyzed on denaturing polyacrylamide gel.

In a similar fashion described as above, piRNAs can also be targeted by engineered RNA sponges. piRNAs are 23-36 nt RNAs that act as guides for a mammalian-specific class of Argonaute proteins known as PIWI proteins. piRNAs are located in the nucleus and cytoplasm and are almost exclusively expressed in gonads. Their function is to preserve genome integrity in germline cells by recognizing and silencing jumping genes post-transcriptionally which may otherwise alter DNA resulting in sterility.

Unlike microRNAs, piRNAs do not require Dicer endonuclease activity for processing. Instead, they rely on RNase type III enzymes to convert double-stranded RNA precursors into functional mature piRNAs. These form piRNA-induced silencing complexes (piRISCs) which largely regulate transposon expression and mediate transcriptional gene silencing.

### Example 3: Targeting Engineered RNA to Different Subcellular Compartments

### Nuclear Localization

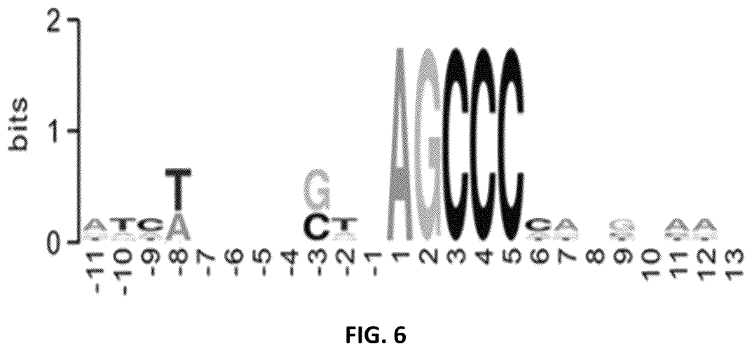

Nuclear localization motifs can be generated to direct engineered RNA to the cell nucleus. Several sequence motifs found on long non-coding RNAs are known to induce nuclear localization. Some of these are regions spanning 100-1000 nt and include 1) a 156 bp repeating RNA domain (RRD) that occurs 8 times in exons of the human lncRNA Firre transcript; 2) large fragments of regions E (1079 nt) and M (1003 nt) of MALATI lncRNA; 3) a miRNA-29b 3′ terminal motif: AGNGUN (where N is any nucleotide) which confers nuclear localization of a functional siRNA directed against luciferase; and 4) a BORG lncRNA having a nuclear-retention motif of 5 nt (AGCCC) with 2 sequence restrictions at positions −8 (A or T) and −3 (G or C) relative to the start of the pentamer. See FIG. 6 depicting the AGCCC motif responsible for nuclear localization.

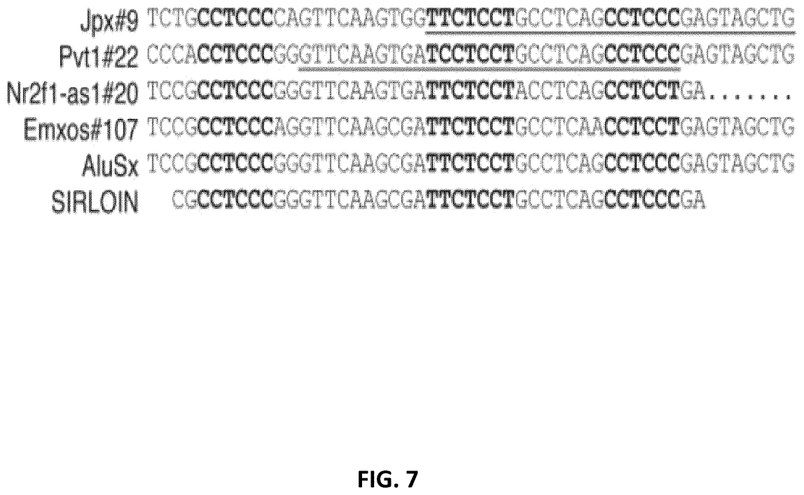

Previous work has shown a 42-nt motif named SINE-derived nuclear-RNA-localization element (SIRLOIN) was identified to be strongly associated with nuclear localization. It is derived from an Alu repeat containing three cytosine-rich elements, two of which matched the consensus RCCTCCC (where R is A/G). The SIRLOIN motif was shown to drive nuclear enrichment of GFP mRNA measured by qPCR and imaging flow cytometry. FIG. 7 depicts sequence alignment of the four most effective regions associated with nuclear localization derived from lncRNAs JPX, PVT1, NR2F1-AS1 and Emxos. The consensus sequence of the AluSx repeat family and the SIRLOIN element are shown. C/T-rich hexamers in bold text.

These elements can be incorporated into engineered nucleic acids (e.g., sfRNAs) for nuclear localization.

### Exosome Localization

RBP hnRNPA2B1 has been reported to regulate miRNA trafficking into exosomes by binding conserved motifs: GGAG and CCCU. Additionally, these specific motifs have been found present in the 5′ end of IncARSR (lncRNA Activated in RCC with Sunitinib Resistance), predominantly localized in cytoplasm. This allowed for IncARSR packaging into exosomes mediated by hnRNPA2B1 binding.

Previous research has shown, three linear 8-mer motifs: ACCAGCCU, CAGUGAGC, UAAUCCCA to be enriched in exosomal RNAs. RBPs YB-1 and NSUN2 are these motif binding partners in the cytosolic cell extract of HEK293. Both proteins are present in exosomes secreted by HEK293 cells. Moreover, these motifs are found in a large representative set of RNA species including mRNAs revealed by next generation sequencing of total exosomal RNA.

Motif preference for circRNAs among exosome fractions of HepG2 cells was previously researched and determined that the exosome could selectively package circRNAs containing the purine-rich 5′-GMWGVWGRAG-3′ motif (SEQ ID NO: 587).

These motifs can be used investigate engineered RNA sponge localization into exosomes. For example, an sfRNA can comprise a motif for exosome localization. After transfection, RNA would be extracted from exosomes isolated from cells and quantified by RT-qPCR to determine if sfRNA was localized to the exosome.

### Chromatin Localization

To determine chromatin localization, fragments of lncRNA and mRNA candidates enriched in the chromatin fraction of human and mouse cells are fused to a cytoplasmic GFP reporter on a sfRNA to identify chromatin-enriched RNA fragments. Additionally, a 7-nt motif (GGUGAGU) resembling a Ul snRNA-recognition site is fused to a cytoplasmic GFP reporter on a sfRNA to identify chromatin-enriched RNA fragments. Engineered sfRNA localization to chromatin is determined by subcellular fractionation.

### Example 4: Design Strategy for Engineered Nucleic Acids

It has been primarily adopted that miRNA function is determined by perfect complementarity between a specific 2-8 nt “seed” region at the 5′ end of the mature miRNA and the target MBS. These canonical target sites are important for initiating a miRNA-target duplex dictated by seed-pairing rules. However, evidence of functional non-canonical target sites has surfaced since 2006. Further analysis of non-canonical interactions led to a general “pivot pairing” rule. The rule indicates that the nucleotide chosen to be inserted at position 6 of the miRNA binding site to create a bulge should be determined by base-pairing competency to a nucleotide in position 6 of the miRNA (termed “pivot” nucleotide). This enables 5 consecutive base pairs (between positions 2-6) that confers thermodynamic stability required for miRNA-target duplex initiation. It is worthwhile noting that the first nucleotide of miRNA is unavailable for pairing as it is not part of the prearranged A-form helix.

In the RISC complex, as miRNA loads onto Ago2, it gets reshaped to overcome kinks that are present at nucleotides 7 and 10 of the miRNA due to Ago2 structure. In theory, the first 5 consecutive base pairs, between nucleotides 2-6, get prearranged into A-form helical structures that favor base-pairing. Following formation of a base-paired helix, a structural shift from the RNA helix at position 7 is required to overcome the kink caused by Ago2 structure. Thus, the “pivot pairing” rule enables the stable formation of the initial 5 consecutive base pairs, and following a subsequent shift at position 7, extended hybridization occurs with the rest of the miRNA towards 3′ end.

In addition to inserting a nucleotide complementary to the miRNA pivot nucleotide, substitution of other nucleotides may still allow for favorable transitional nucleation stability despite only forming 4 consecutive base pairs. Evidence has suggested that downstream compensatory interactions with 3′ miRNA region could contribute to this stability. Thus each of the four nucleotides A, G, C or U were incorporated when creating imperfect seed complementarity in target sites to investigate which nucleotide would be most favorable in the various miRNA contexts as described next.

### Results

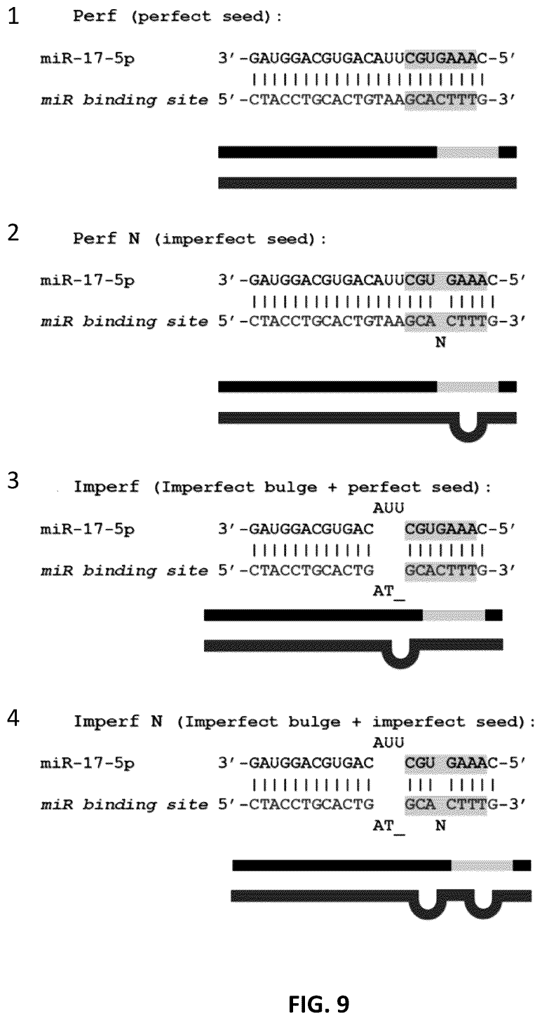

Snowflake constructs harboring each miRNA binding site (MBS) type illustrated in FIG. 9 were generated to target hsa-miR-17-5p, hsa-miR-18a-5p, hsa-miR-132-3p, hsa-miR-155-5p. The four main types of MBS studied here are 100% perfect complementarity (Perf) (FIG. 9, number 1), imperfect seed complementarity created by inserting either an A, G, C or U nucleotide in position 6 of the miRNA (Perf N) (FIG. 9, number 2), imperfect bulge created by one deletion and two mismatches at positions 9-11 of the miRNA (Imperf) (FIG. 9, number 3) and imperfect bulge at position 9-11 together with an imperfect seed complementarity (Imperf N) (FIG. 9, number 4).

The functional efficacy between these MBS types within snowflakes was validated in a luciferase rescue assay. First, six Perf or Imperf MBS for each hsa-miR-17-5p, hsa-miR-18a-5p, hsa-miR-132-3p, hsa-miR-155-5p were inserted into the 3′-UTR of a Renilla luciferase control construct from a dual-luciferase reporter system (control Perf miR x6 or control Imperf miR x6).

Accordingly, a luciferase rescue reporter assay was performed using a dual reporter construct with six Perf or Imperf miRNA binding sites inserted into the 3′-UTR of the Renilla luciferase gene. HEK293T cells were co-transfected with control reporter plasmids, miR-155 or miR-132 mimics where necessary and respective snowflake RNAs for 48 h, to determine the effect of snowflakes with different miRNA binding site types targeting (A) miR-155, (B) miR-132, (C) miR-17 and (D) miR-18a. (n=3); * P<0.05, **P<0.01, ***P<0.001, ****P<0.0001 (Perf/Imperf snowflake versus control Perf/Imperf miR x6 with mimics) for (A-B), (Perf/Imperf snowflake versus control Perf/Imperf miR x6) for (C-D). One-way ANOVA with Bonferroni correction.

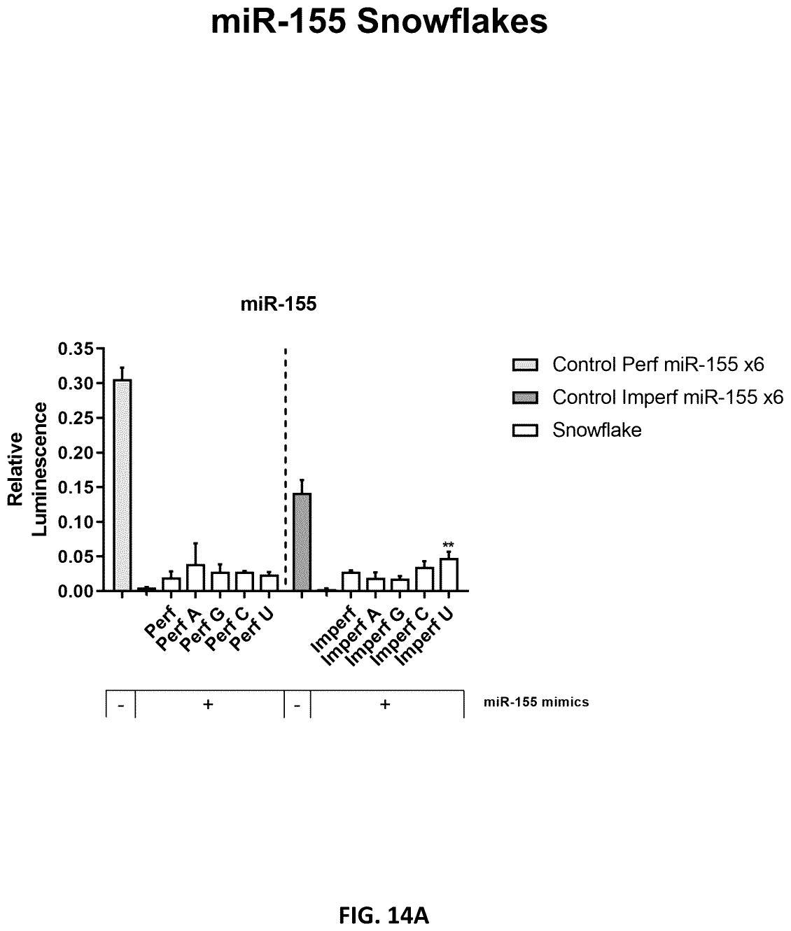

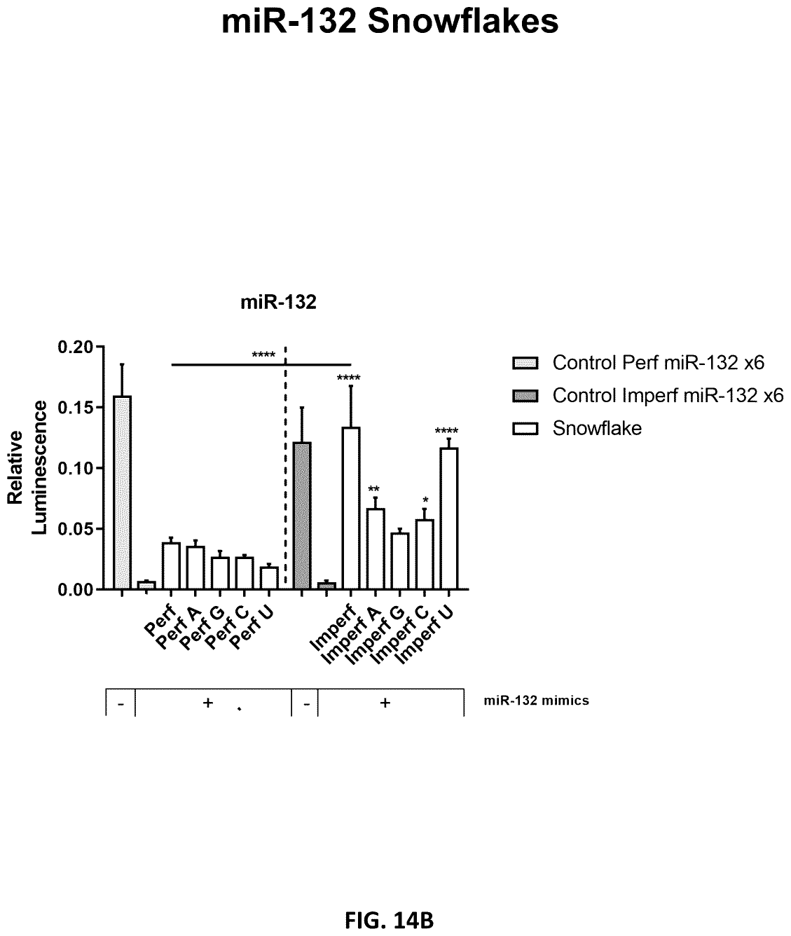

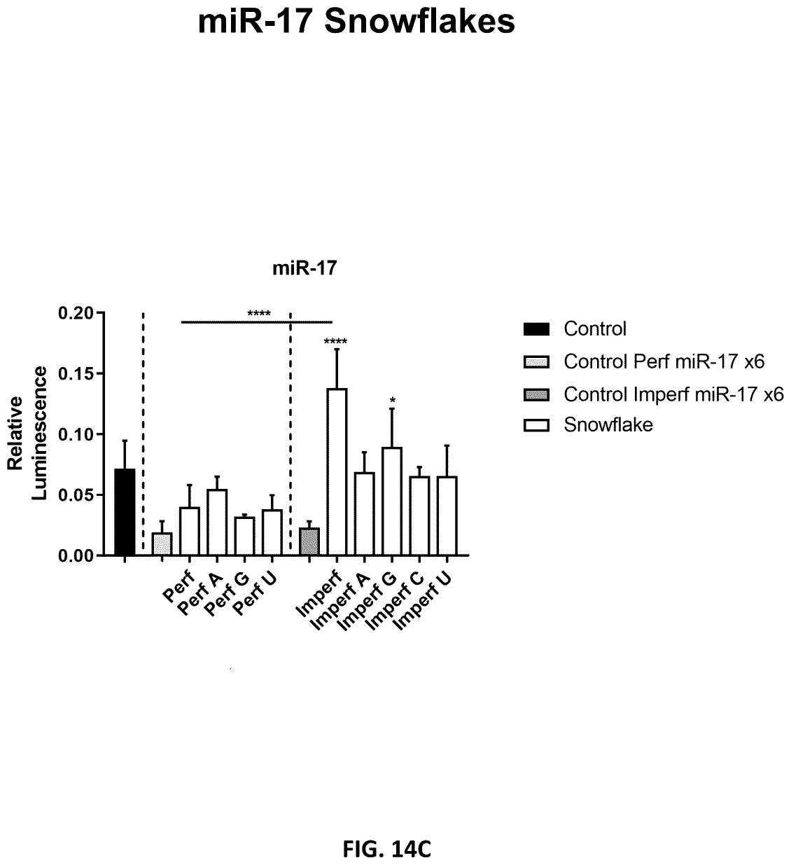

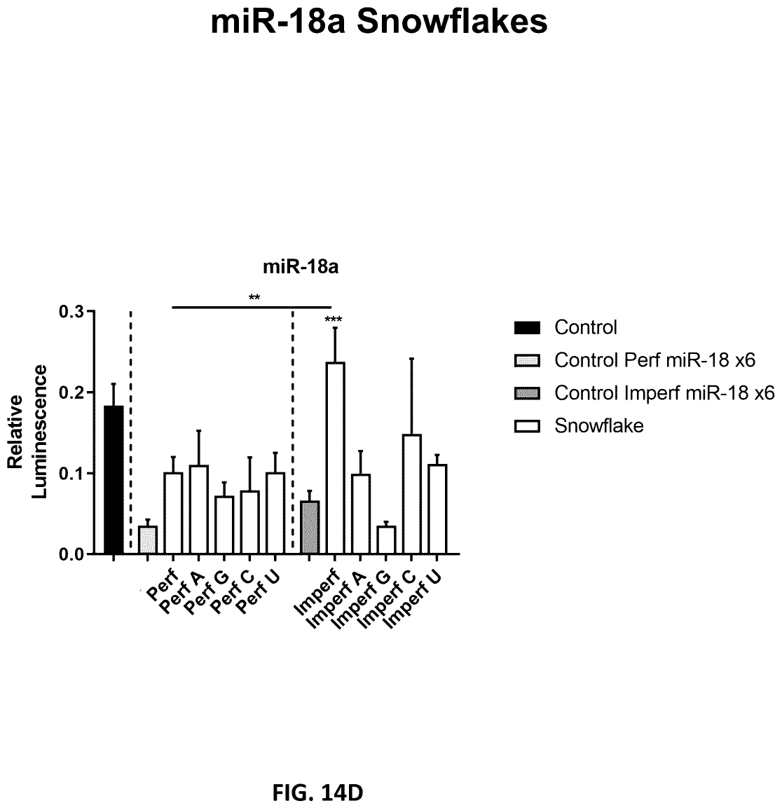

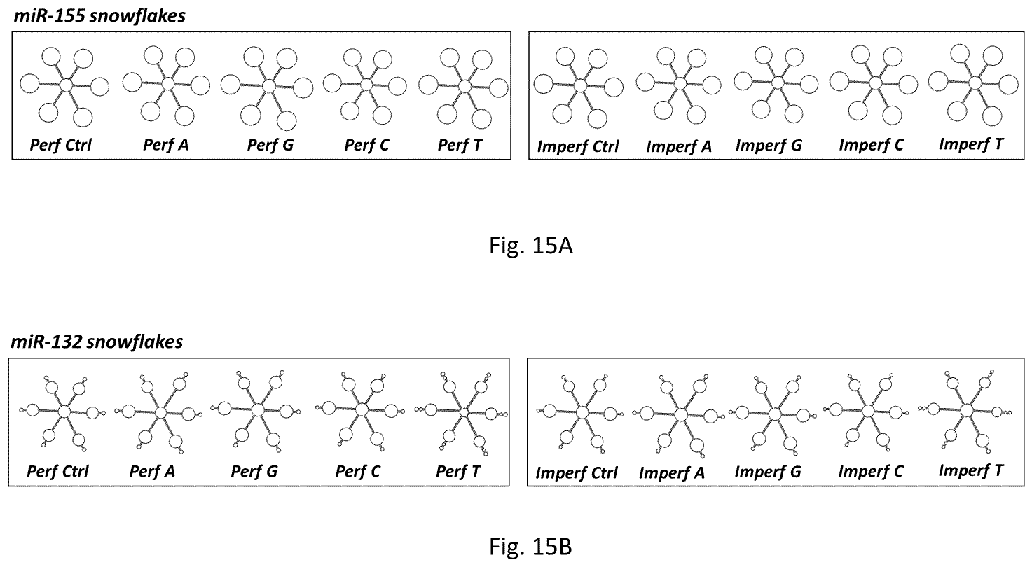

Co-transfecting the respective control constructs with miR-155 mimics in HEK293T cells resulted in a significant reduction in Renilla activity (FIG. 14A). Upon introduction of snowflakes carrying miR-155 binding sites, Renilla activity trended to increase although these changes did not reach statistical significance except for snowflakes carrying Imperf U binding sites (bulge at positions 9-12 nt together with a bulge in the seed region created by inserting U nucleotide at position 6) (FIG. 14A). No significant difference was seen between Perf and Imperf snowflakes (FIG. 14A).

Co-transfecting control constructs with miR-132 mimics in HEK293T cells resulted in a significant reduction in Renilla activity as anticipated (FIG. 14B). Upon introduction of synthetic snowflake carrying miR-132 binding sites, Renilla activity was significantly rescued by Imperf, Imperf A, Imperf C and Imperf U miR-132 snowflakes (FIG. 14B). Imperf snowflakes show a greater rescue effect than Perf snowflakes (FIG. 14B). Notably, Imperf and Imperf U snowflakes showed similar rescue effects suggesting that having perfect complementarity within the seed region is not entirely necessary for miRNA inhibition in snowflake designs. Imperf U showed the greatest rescue effect followed by nucleotides A (Imperf A) and C (Imperf C). Imperf G increased Renilla activity although not statistically significant (FIG. 14B).

Both miR-17 and miR-18a are endogenously expressed in HEK293T cells. Hence, miRNA mimics were not required for the following luciferase rescue assays.

Transfecting miR-17 or miR-18 synthetic RNA control constructs in HEK293T cells resulted in a reduction in Renilla activity (FIG. 14C and FIG. 14D). Upon introduction of synthetic RNA snowflake carrying miR-17 binding sites, Renilla activity was significantly rescued by Imperf and Imperf G miR-17 snowflakes (FIG. 14C). No statistically significant rescue was detected, although trends towards rescue were observed for the rest of the miR-17 snowflakes. These trends appeared greater in Imperfect versus Perfect groups. Of note, Imperf snowflake show a significantly greater rescue effect than Perf snowflake (FIG. 14C).

Imperf miR-18 snowflake showed a significant rescue effect compared to control construct (FIG. 14D). Rescue effects for all other miR-18 tested snowflakes, except Imperf G, demonstrate trends towards rescue although not statistically significant (FIG. 14D).

DNA sequences for the RNAs used in the experiments above are found in Table 7, SEQ ID NOs: 175-252, Table 7 also indicates the target RNA and design of the engineered nucleic acid. SEQ ID NOs: 214-252 contain the DNA sequences of the synthesized RNA sequence which comprises “ggg” added to the 5′ end of the sequence. The synthetic RNA sequences used in the experiments above are found in Table 8, SEQ ID NOs: 253-330 contains the same information but show the RNA sequences. SEQ ID NOs: 292-330 contain the synthesized RNA sequences which comprise “ggg” added to the 5′ end of the sequence.

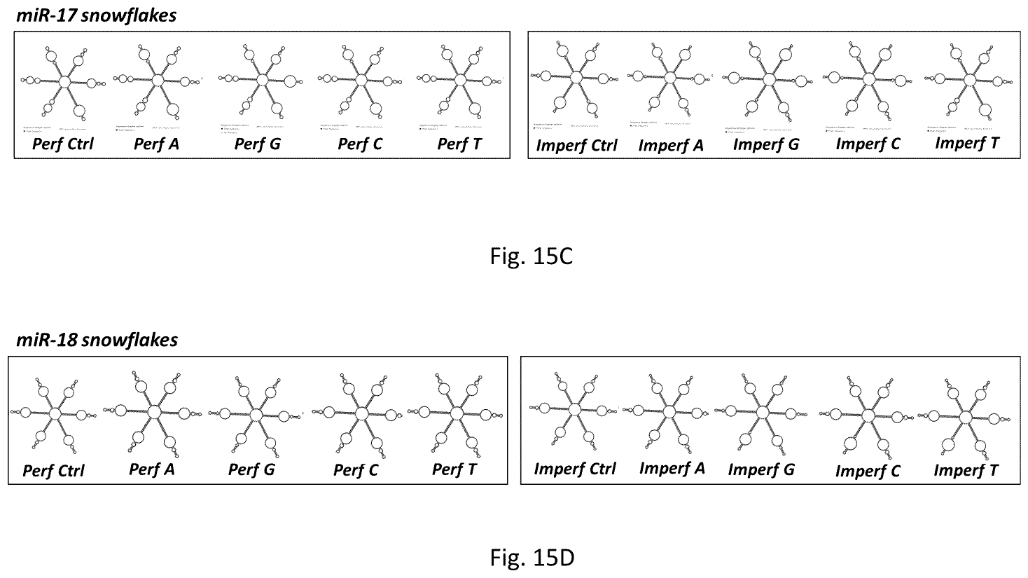

Overall, these findings indicate that different types of miRNA binding sites within snowflakes exert varying effects for inhibition of miRNAs: miR-155, miR-132, miR-17, miR-18a. In general, Imperf snowflakes show a greater efficacy compared to Perf snowflakes. However, this finding was consistent with Imperf snowflakes designed for the inhibition of miR-132, miR-17 and miR-18a, except for miR-155. This discrepancy could be attributed to the snowflake structure (FIGS. 15A-D). Unstructured loops containing miRNA binding sites could only be designed for miR-155 whereas short stems were present in loops carrying binding sites for miR-132, miR-17 and miR-18a (FIGS. 15A-D). The short stems are a result of self-complementary within target site and this is dependent upon each unique miRNA sequence. Thus, there could be a scenario where differences between Perf and Imperf binding sites cease to exist in unstructured loops (FIG. 15A) whereas differences between Perf and Imperf binding sites are more apparent in loops with short stem regions (FIGS. 15B-D). Further testing in which snowflakes designed for miRNAs, other than miR-155, that allow for unstructured loop formation is required to make a clear deduction.

Additionally, the type of nucleotide inserted at position 6 to create an imperfect seed complementarity in the binding site showed varying effects for each miRNA tested. For miR-155, Imperf U snowflake showed the greatest rescue effect compared to Imperf A/G/C. For miR-132, Imperf U snowflake showed the greatest efficacy followed by Imperf A snowflake. For miR-17, Imperf G snowflake performed better than snowflakes carrying imperf target sites with A/C/T insertions. For miR-18, Imperf G showed no rescue while Imperf A/C/U snowflakes trended towards a rescue effect. These results likely indicate the need to carry out testing of binding site types with various nucleotide seed insertions for each unique miRNA prior to deciding which MBS would be best suited for use in the snowflake design as a general trend cannot be deduced yet.

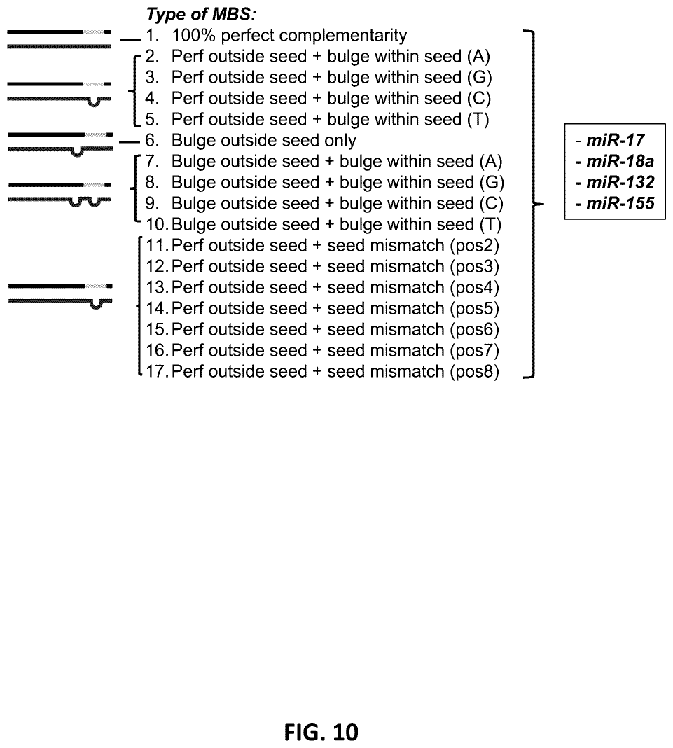

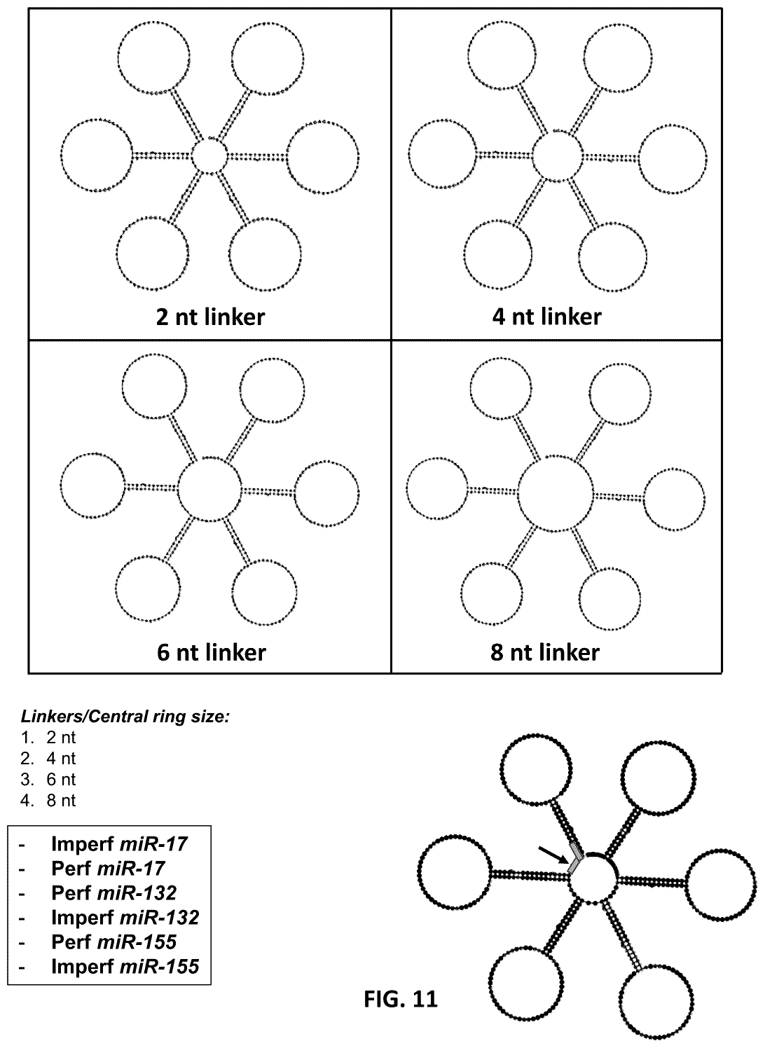

This design may be employed for a variety of binding sites for various miRNAs such as miR-17, miR-18a, miR-132, miR-155 is shown in FIG. 10 and (SEQ ID NOs: 79-174, Table 6). Table 6 provides the DNA sequence of the binding sites for various miRNAs and the description of the binding sites (SEQ ID NOs: 79-174 and also provides the RNA sequence of binding sites for various miRNAs (SEQ ID NOs: 331-426). The description provides details on the complementarity and design of the binding sites. Any DNA sequence provided herein also includes the RNA sequence wherein all T's are substituted for U's. An engineered polynucleotide disclosed herein can be engineered to incorporate any one of SEQ ID NOs 79-174 (DNA sequences) or 331-426 (RNA sequences).

TABLE 6
miRNA binding site sequences

| Description | Sequence |
|---|---|
| miR-17-5p Perf seed | CTACCTGCACTGTAAGCACTTTG (SEQ ID NO: 79) |
| 100% perfect complementarity | CUACCUGCACUGUAAGCACUUUG (SEQ ID NO: 331) |
| miR-17-5p Perf A | CTACCTGCACTGTAAGCAaCTTTG (SEQ ID NO: 80) |
| Perf outside seed + bulge within seed (A) | CUACCUGCACUGUAAGCAaCUUUG (SEQ ID NO: 332) |
| miR-17-5p Perf G | CTACCTGCACTGTAAGCAgCTTTG (SEQ ID NO: 81) |
| Perf outside seed + bulge within seed (G) | CUACCUGCACUGUAAGCAgCUUUG (SEQ ID NO: 333) |
| miR-17-5p Perf C | CTACCTGCACTGTAAGCAcCTTTG (SEQ ID NO: 82) |
| Perf outside seed + bulge within seed (C) | CUACCUGCACUGUAAGCAcCUUUG (SEQ ID NO: 334 |
| miR-17-5p Perf T | CTACCTGCACTGTAAGCAtCTTTG (SEQ ID NO: 83) |
| Perf outside seed + bulge within seed (T) | CUACCUGCACUGUAAGCAuCUUUG (SEQ ID NO: 335) |
| miR-17-5p Imperf seed | CTACCTGCACTGATGCACTTTG (SEQ ID NO: 84) |
| Bulge outside seed only | CUACCUGCACUGAUGCACUUUG (SEQ ID NO: 336) |
| miR-17-5p Imperf A | CTACCTGCACTGATGCAaCTTTG (SEQ ID NO: 85) |
| Bulge outside seed + bulge within seed (A) | CUACCUGCACUGAUGCAaCUUUG (SEQ ID NO: 337) |
| miR-17-5p Imperf G | CTACCTGCACTGATGCAgCTTTG (SEQ ID NO: 86) |
| Bulge outside seed + bulge within seed (G) | CUACCUGCACUGAUGCAgCUUUG (SEQ ID NO: 338) |
| miR-17-5p Imperf C | CTACCTGCACTGATGCAcCTTTG (SEQ ID NO: 87) |
| Bulge outside seed + bulge within seed (C) | CUACCUGCACUGAUGCAcCUUUG (SEQ ID NO: 339) |
| miR-17-5p Imperf T | CTACCTGCACTGATGCAtCTTTG (SEQ ID NO: 88) |
| Bulge outside seed + bulge within seed (T) | CUACCUGCACUGAUGCAuCUUUG (SEQ ID NO: 340) |
| miR-17-5p Perf pos2 | CTACCTGCACTGTAAGCACTTAG (SEQ ID NO: 89) |
| Perf outside seed + seed mismatch (pos2) | CUACCUGCACUGUAAGCACUUAG (SEQ ID NO: 341) |
| miR-17-5p Perf pos3 | CTACCTGCACTGTAAGCACTATG (SEQ ID NO: 90) |
| Perf outside seed + seed mismatch (pos3) | CUACCUGCACUGUAAGCACUAUG (SEQ ID NO: 342) |
| miR-17-5p Perf pos4 | CTACCTGCACTGTAAGCACATTG (SEQ ID NO: 91) |
| Perf outside seed + seed mismatch (pos4) | CUACCUGCACUGUAAGCACAUUG (SEQ ID NO: 343) |
| miR-17-5p Perf pos5 | CTACCTGCACTGTAAGCAGTTTG (SEQ ID NO: 92) |
| Perf outside seed + seed mismatch (pos5) | CUACCUGCACUGUAAGCAGUUUG (SEQ ID NO: 344) |
| miR-17-5p Perf pos6 | CTACCTGCACTGTAAGCTCTTTG (SEQ ID NO: 93) |
| Perf outside seed + seed mismatch (pos6) | CUACCUGCACUGUAAGCUCUUUG (SEQ ID NO: 345) |
| miR-17-5p Perf pos7 | CTACCTGCACTGTAAGGACTTTG (SEQ ID NO: 94) |
| Perf outside seed + seed mismatch (pos7) | CUACCUGCACUGUAAGGACUUUG (SEQ ID NO: 346) |
| miR-17-5p Perf pos8 | CTACCTGCACTGTAACCACTTTG (SEQ ID NO: 95) |
| Perf outside seed + seed mismatch (pos8) | CUACCUGCACUGUAACCACUUUG (SEQ ID NO: 347) |
| miR-17-5p Imperf pos2 | CTACCTGCACTGATGCACTTAG (SEQ ID NO: 96) |
| Imperf outside seed + seed mismatch (pos2) | CUACCUGCACUGAUGCACUUAG (SEQ ID NO: 348) |
| miR-17-5p Imperf pos3 | CTACCTGCACTGATGCACTATG (SEQ ID NO: 97) |
| Imperf outside seed + seed mismatch (pos3) | CUACCUGCACUGAUGCACUAUG (SEQ ID NO: 349) |
| miR-17-5p Imperf pos4 | CTACCTGCACTGATGCACATTG (SEQ ID NO: 98) |
| Imperf outside seed + seed mismatch (pos4) | CUACCUGCACUGAUGCACAUUG (SEQ ID NO: 350) |
| miR-17-5p Imperf pos5 | CTACCTGCACTGATGCAGTTTG (SEQ ID NO: 99) |
| Imperf outside seed + seed mismatch (pos5) | CUACCUGCACUGAUGCAGUUUG (SEQ ID NO: 351) |
| miR-17-5p Imperf pos6 | CTACCTGCACTGATGCTCTTTG (SEQ ID NO: 100) |
| Imperf outside seed + seed mismatch (pos6) | CUACCUGCACUGAUGCUCUUUG (SEQ ID NO: 352) |
| miR-17-5p Imperf pos7 | CTACCTGCACTGATGGACTTTG (SEQ ID NO: 101) |
| Imperf outside seed + seed mismatch (pos7) | CUACCUGCACUGAUGGACUUUG (SEQ ID NO: 353) |
| miR-17-5p Imperf pos8 | CTACCTGCACTGATCCACTTTG (SEQ ID NO: 102) |
| Imperf outside seed + seed mismatch (pos8) | CUACCUGCACUGAUCCACUUUG (SEQ ID NO: 354) |
| miR-18a-5p Perf seed | CTATCTGCACTAGATGCACCTTA (SEQ ID NO: 103) |
| 100% perfect complementarity | CUAUCUGCACUAGAUGCACCUUA (SEQ ID NO: 355) |
| miR-18a-5p Perf A | CTATCTGCACTAGATGCAaCCTTA (SEQ ID NO: 104) |
| Perf outside seed + bulge within seed (A) | CUAUCUGCACUAGAUGCAaCCUUA (SEQ ID NO: 356) |
| miR-18a-5p Perf G | CTATCTGCACTAGATGCAgCCTTA (SEQ ID NO: 105) |
| Perf outside seed + bulge within seed (G) | CUAUCUGCACUAGAUGCAgCCUUA (SEQ ID NO: 357) |
| miR-18a-5p Perf C | CTATCTGCACTAGATGCAcCCTTA (SEQ ID NO: 106) |
| Perf outside seed + bulge within seed (C) | CUAUCUGCACUAGAUGCAcCCUUA (SEQ ID NO: 358) |
| miR-18a-5p Perf T | CTATCTGCACTAGATGCAtCCTTA (SEQ ID NO: 107) |
| Perf outside seed + bulge within seed (T) | CUAUCUGCACUAGAUGCAuCCUUA (SEQ ID NO: 359) |
| miR-18a-5p Imperf seed | CTATCTGCACTACTGCACCTTA (SEQ ID NO: 108) |
| Bulge outside seed only | CUAUCUGCACUACUGCACCUUA (SEQ ID NO: 360) |
| miR-18a-5p Imperf A | CTATCTGCACTACTGCAaCCTTA (SEQ ID NO: 109) |
| Bulge outside seed + bulge within seed (A) | CUAUCUGCACUACUGCAaCCUUA (SEQ ID NO: 361) |
| miR-18a-5p Imperf G | CTATCTGCACTACTGCAgCCTTA (SEQ ID NO: 110) |
| Bulge outside seed + bulge within seed (G) | CUAUCUGCACUACUGCAgCCUUA (SEQ ID NO: 362) |
| miR-18a-5p Imperf C | CTATCTGCACTACTGCAcCCTTA (SEQ ID NO: 111) |
| Bulge outside seed + bulge within seed (C) | CUAUCUGCACUACUGCAcCCUUA (SEQ ID NO: 363) |
| miR-18a-5p Imperf T | CTATCTGCACTACTGCAtCCTTA (SEQ ID NO: 112) |
| Bulge outside seed + bulge within seed (T) | CUAUCUGCACUACUGCAuCCUUA (SEQ ID NO: 364) |
| miR-18a-5p Perf pos2 | CTATCTGCACTAGATGCACCTAA (SEQ ID NO: 113) |
| Perf outside seed + seed mismatch (pos2) | CUAUCUGCACUAGAUGCACCUAA (SEQ ID NO: 365) |
| miR-18a-5p Perf pos3 | CTATCTGCACTAGATGCACCATA (SEQ ID NO: 114) |
| Perf outside seed + seed mismatch (pos3) | CUAUCUGCACUAGAUGCACCAUA (SEQ ID NO: 366) |
| miR-18a-5p Perf pos4 | CTATCTGCACTAGATGCACGTTA (SEQ ID NO: 115) |
| Perf outside seed + seed mismatch (pos4) | CUAUCUGCACUAGAUGCACGUUA (SEQ ID NO: 367) |
| miR-18a-5p Perf pos5 | CTATCTGCACTAGATGCAGCTTA (SEQ ID NO: 116) |
| Perf outside seed + seed mismatch (pos5) | CUAUCUGCACUAGAUGCAGCUUA (SEQ ID NO: 368) |
| miR-18a-5p Perf pos6 | CTATCTGCACTAGATGCTCCTTA (SEQ ID NO: 117) |
| Perf outside seed + seed mismatch (pos6) | CUAUCUGCACUAGAUGCUCCUUA (SEQ ID NO: 369) |
| miR-18a-5p Perf pos7 | CTATCTGCACTAGATGGACCTTA (SEQ ID NO: 118) |
| Perf outside seed + seed mismatch (pos7) | CUAUCUGCACUAGAUGGACCUUA (SEQ ID NO: 370) |
| miR-18a-5p Perf pos8 | CTATCTGCACTAGATCCACCTTA (SEQ ID NO: 119) |
| Perf outside seed + seed mismatch (pos8) | CUAUCUGCACUAGAUCCACCUUA (SEQ ID NO: 371) |
| miR-18a-5p Imperf pos2 | CTATCTGCACTACTGCACCTAA (SEQ ID NO: 120) |
| Imperf outside seed + seed mismatch (pos2) | CUAUCUGCACUACUGCACCUAA (SEQ ID NO: 372) |
| miR-18a-5p Imperf pos3 | CTATCTGCACTACTGCACCATA (SEQ ID NO: 121) |
| Imperf outside seed + seed mismatch (pos3) | CUAUCUGCACUACUGCACCAUA (SEQ ID NO: 373) |
| miR-18a-5p Imperf pos4 | CTATCTGCACTACTGCACGTTA (SEQ ID NO: 122) |
| Imperf outside seed + seed mismatch (pos4) | CUAUCUGCACUACUGCACGUUA (SEQ ID NO: 374) |
| miR-18a-5p Imperf pos5 | CTATCTGCACTACTGCAGCTTA (SEQ ID NO: 123) |
| Imperf outside seed + seed mismatch (pos5) | CUAUCUGCACUACUGCAGCUUA (SEQ ID NO: 375) |
| miR-18a-5p Imperf pos6 | CTATCTGCACTACTGCTCCTTA (SEQ ID NO: 124) |
| Imperf outside seed + seed mismatch (pos6) | CUAUCUGCACUACUGCUCCUUA (SEQ ID NO: 376) |
| miR-18a-5p Imperf pos7 | CTATCTGCACTACTGGACCTTA (SEQ ID NO: 125) |
| Imperf outside seed + seed mismatch (pos7) | CUAUCUGCACUACUGGACCUUA (SEQ ID NO: 377) |
| miR-18a-5p Imperf pos8 | CTATCTGCACTACTCCACCTTA (SEQ ID NO: 126) |
| Imperf outside seed + seed mismatch (pos8) | CUAUCUGCACUACUCCACCUUA (SEQ ID NO: 378) |
| miR-132-3p Perf seed | CGACCATGGCTGTAGACTGTTA (SEQ ID NO: 127) |
| 100% perfect complementarity | CGACCAUGGCUGUAGACUGUUA (SEQ ID NO: 379) |
| miR-132-3p Perf A | CGACCATGGCTGTAGACaTGTTA (SEQ ID NO: 128) |
| Perf outside seed + bulge within seed (A) | CGACCAUGGCUGUAGACaUGUUA (SEQ ID NO: 380) |
| miR-132-3p Perf G | CGACCATGGCTGTAGACgTGTTA (SEQ ID NO: 129) |
| Perf outside seed + bulge within seed (G) | CGACCAUGGCUGUAGACgUGUUA (SEQ ID NO: 381) |
| miR-132-3p Perf C | CGACCATGGCTGTAGACcTGTTA (SEQ ID NO: 130) |
| Perf outside seed + bulge within seed (C) | CGACCAUGGCUGUAGACcUGUUA (SEQ ID NO: 382) |
| miR-132-3p Perf T | CGACCATGGCTGTAGACtTGTTA (SEQ ID NO: 131) |
| Perf outside seed + bulge within seed (T) | CGACCAUGGCUGUAGACuUGUUA (SEQ ID NO: 383) |
| miR-132-3p Imperf seed | CGACCATGGCTCAGACTGTTA (SEQ ID NO: 132) |
| Bulge outside seed only | CGACCAUGGCUCAGACUGUUA (SEQ ID NO: 384) |
| miR-132-3p Imperf A | CGACCATGGCTCAGACaTGTTA (SEQ ID NO: 133) |
| Bulge outside seed + bulge within seed (A) | CGACCAUGGCUCAGACaUGUUA (SEQ ID NO: 385) |
| miR-132-3p Imperf G | CGACCATGGCTCAGACgTGTTA (SEQ ID NO: 134) |
| Bulge outside seed + bulge within seed (G) | CGACCAUGGCUCAGACgUGUUA (SEQ ID NO: 386) |
| miR-132-3p Imperf C | CGACCATGGCTCAGACcTGTTA (SEQ ID NO: 135) |
| Bulge outside seed + bulge within seed (C) | CGACCAUGGCUCAGACcUGUUA (SEQ ID NO: 387) |
| miR-132-3p Imperf T | CGACCATGGCTCAGACtTGTTA (SEQ ID NO: 136) |
| Bulge outside seed + bulge within seed (T) | CGACCAUGGCUCAGACuUGUUA (SEQ ID NO: 388) |
| miR-132-3p Perf pos2 | CGACCATGGCTGTAGACTGTAA (SEQ ID NO: 137) |
| Perf outside seed + seed mismatch (pos2) | CGACCAUGGCUGUAGACUGUAA (SEQ ID NO: 389) |
| miR-132-3p Perf pos3 | CGACCATGGCTGTAGACTGATA (SEQ ID NO: 138) |
| Perf outside seed + seed mismatch (pos3) | CGACCAUGGCUGUAGACUGAUA (SEQ ID NO: 390) |
| miR-132-3p Perf pos4 | CGACCATGGCTGTAGACTCTTA (SEQ ID NO: 139) |
| Perf outside seed + seed mismatch (pos4) | CGACCAUGGCUGUAGACUCUUA (SEQ ID NO: 391) |
| miR-132-3p Perf pos5 | CGACCATGGCTGTAGACAGTTA (SEQ ID NO: 140) |
| Perf outside seed + seed mismatch (pos5) | CGACCAUGGCUGUAGACAGUUA (SEQ ID NO: 392) |
| miR-132-3p Perf pos6 | CGACCATGGCTGTAGAGTGTTA (SEQ ID NO: 141) |
| Perf outside seed + seed mismatch (pos6) | CGACCAUGGCUGUAGAGUGUUA (SEQ ID NO: 393) |
| miR-132-3p Perf pos7 | CGACCATGGCTGTAGTCTGTTA (SEQ ID NO: 142) |
| Perf outside seed + seed mismatch (pos7) | CGACCAUGGCUGUAGUCUGUUA (SEQ ID NO: 394) |
| miR-132-3p Perf pos8 | CGACCATGGCTGTACACTGTTA (SEQ ID NO: 143) |
| Perf outside seed + seed mismatch (pos8) | CGACCAUGGCUGUACACUGUUA (SEQ ID NO: 395) |
| miR-132-3p Imperf pos2 | CGACCATGGCTCAGACTGTAA (SEQ ID NO: 144) |
| Imperf outside seed + seed mismatch (pos2) | CGACCAUGGCUCAGACUGUAA (SEQ ID NO: 396) |
| miR-132-3p Imperf pos3 | CGACCATGGCTCAGACTGATA (SEQ ID NO: 145) |
| Imperf outside seed + seed mismatch (pos3) | CGACCAUGGCUCAGACUGAUA (SEQ ID NO: 397) |
| miR-132-3p Imperf pos4 | CGACCATGGCTCAGACTCTTA (SEQ ID NO: 146) |
| Imperf outside seed + seed mismatch (pos4) | CGACCAUGGCUCAGACUCUUA (SEQ ID NO: 398) |
| miR-132-3p Imperf pos5 | CGACCATGGCTCAGACAGTTA (SEQ ID NO: 147) |
| Imperf outside seed + seed mismatch (pos5) | CGACCAUGGCUCAGACAGUUA (SEQ ID NO: 399) |
| miR-132-3p Imperf pos6 | CGACCATGGCTCAGAGTGTTA (SEQ ID NO: 148) |
| Imperf outside seed + seed mismatch (pos6) | CGACCAUGGCUCAGAGUGUUA (SEQ ID NO: 400) |
| miR-132-3p Imperf pos7 | CGACCATGGCTCAGTCTGTTA (SEQ ID NO: 149) |
| Imperf outside seed + seed mismatch (pos7) | CGACCAUGGCUCAGUCUGUUA (SEQ ID NO: 401) |
| miR-132-3p Imperf pos8 | CGACCATGGCTCACACTGTTA (SEQ ID NO: 150) |
| Imperf outside seed + seed mismatch (pos8) | CGACCAUGGCUCACACUGUUA (SEQ ID NO: 402) |
| miR-155-5p Perf seed | AACCCCTATCACGATTAGCATTAA (SEQ ID NO: 151) |
| 100% perfect complementarity | AACCCCUAUCACGAUUAGCAUUAA (SEQ ID NO: 403) |
| miR-155-5p Perf A | AACCCCTATCACGATTAGCaATTAA (SEQ ID NO: 152) |
| Perf outside seed + bulge within seed (A) | AACCCCUAUCACGAUUAGCaAUUAA (SEQ ID NO: 404) |
| miR-155-5p Perf G | AACCCCTATCACGATTAGCgATTAA (SEQ ID NO: 153) |
| Perf outside seed + bulge within seed (G) | AACCCCUAUCACGAUUAGCgAUUAA (SEQ ID NO: 405) |
| miR-155-5p Perf C | AACCCCTATCACGATTAGCcATTAA (SEQ ID NO: 154) |
| Perf outside seed + bulge within seed (C) | AACCCCUAUCACGAUUAGCcAUUAA (SEQ ID NO: 406) |
| miR-155-5p Perf T | AACCCCTATCACGATTAGCtATTAA (SEQ ID NO: 155) |
| Perf outside seed + bulge within seed (T) | AACCCCUAUCACGAUUAGCuAUUAA (SEQ ID NO: 407) |
| miR-155-5p Imperf seed | AACCCCTATCACGTAAGCATTAA (SEQ ID NO: 156) |
| Bulge outside seed only | AACCCCUAUCACGUAAGCAUUAA (SEQ ID NO: 408) |
| miR-155-5p Imperf A | AACCCCTATCACGTAAGCaATTAA (SEQ ID NO: 157) |
| Bulge outside seed + bulge within seed (A) | AACCCCUAUCACGUAAGCaAUUAA (SEQ ID NO: 409) |
| miR-155-5p Imperf G | AACCCCTATCACGTAAGCgATTAA (SEQ ID NO: 158) |
| Bulge outside seed + bulge within seed (G) | AACCCCUAUCACGUAAGCgAUUAA (SEQ ID NO: 410) |
| miR-155-5p Imperf C | AACCCCTATCACGTAAGCcATTAA (SEQ ID NO: 159) |
| Bulge outside seed + bulge within seed (C) | AACCCCUAUCACGUAAGCcAUUAA (SEQ ID NO: 411) |
| miR-155-5p Imperf T | AACCCCTATCACGTAAGCtATTAA (SEQ ID NO: 160) |
| Bulge outside seed + bulge within seed (T) | AACCCCUAUCACGUAAGCuAUUAA (SEQ ID NO: 412) |
| miR-155-5p Perf pos2 | AACCCCTATCACGATTAGCATTTA (SEQ ID NO: 161) |
| Perf outside seed + seed mismatch (pos2) | AACCCCUAUCACGAUUAGCAUUUA (SEQ ID NO: 413) |
| miR-155-5p Perf pos3 | AACCCCTATCACGATTAGCATAAA (SEQ ID NO: 162) |
| Perf outside seed + seed mismatch (pos3) | AACCCCUAUCACGAUUAGCAUAAA (SEQ ID NO: 414) |
| miR-155-5p Perf pos4 | AACCCCTATCACGATTAGCAATAA (SEQ ID NO: 163) |
| Perf outside seed + seed mismatch (pos4) | AACCCCUAUCACGAUUAGCAAUAA (SEQ ID NO: 415) |
| miR-155-5p Perf pos5 | AACCCCTATCACGATTAGCTTTAA (SEQ ID NO: 164) |
| Perf outside seed + seed mismatch (pos5) | AACCCCUAUCACGAUUAGCUUUAA (SEQ ID NO: 416) |
| miR-155-5p Perf pos6 | AACCCCTATCACGATTAGGATTAA (SEQ ID NO: 165) |
| Perf outside seed + seed mismatch (pos6) | AACCCCUAUCACGAUUAGGAUUAA (SEQ ID NO: 417) |
| miR-155-5p Perf pos7 | AACCCCTATCACGATTACCATTAA (SEQ ID NO: 166) |
| Perf outside seed + seed mismatch (pos7) | AACCCCUAUCACGAUUACCAUUAA (SEQ ID NO: 418) |
| miR-155-5p Perf pos8 | AACCCCTATCACGATTTGCATTAA (SEQ ID NO: 167) |
| Perf outside seed + seed mismatch (pos8) | AACCCCUAUCACGAUUUGCAUUAA (SEQ ID NO: 419) |
| miR-155-5p Imperf pos2 | AACCCCTATCACGTAAGCATTTA (SEQ ID NO: 168) |
| Imperf outside seed + seed mismatch (pos2) | AACCCCUAUCACGUAAGCAUUUA (SEQ ID NO: 420) |
| miR-155-5p Imperf pos3 | AACCCCTATCACGTAAGCATAAA (SEQ ID NO: 169) |
| Imperf outside seed + seed mismatch (pos3) | AACCCCUAUCACGUAAGCAUAAA (SEQ ID NO: 421) |
| miR-155-5p Imperf pos4 | AACCCCTATCACGTAAGCAATAA (SEQ ID NO: 170) |
| Imperf outside seed + seed mismatch (pos4) | AACCCCUAUCACGUAAGCAAUAA (SEQ ID NO: 422) |
| miR-155-5p Imperf pos5 | AACCCCTATCACGTAAGCTTTAA (SEQ ID NO: 171) |
| Imperf outside seed + seed mismatch (pos5) | AACCCCUAUCACGUAAGCUUUAA (SEQ ID NO: 423) |
| miR-155-5p Imperf pos6 | AACCCCTATCACGTAAGGATTAA (SEQ ID NO: 172) |
| Imperf outside seed + seed mismatch (pos6) | AACCCCUAUCACGUAAGGAUUAA (SEQ ID NO: 424) |
| miR-155-5p Imperf pos7 | AACCCCTATCACGTAACCATTAA (SEQ ID NO: 173) |
| Imperf outside seed + seed mismatch (pos7) | AACCCCUAUCACGUAACCAUUAA (SEQ ID NO: 425) |
| miR-155-5p Imperf pos8 | AACCCCTATCACGTATGCATTAA (SEQ ID NO: 174) |
| Imperf outside seed + seed mismatch (pos8) | AACCCCUAUCACGUAUGCAUUAA (SEQ ID NO: 426) |

TABLE 7
Engineered Nucleic Acid Sequences (DNA)

| Sequence | | |
|---|---|---|
| ID | Description | Sequence |
| SEQ ID NO: | miR-132 | tgatatacgaccagtTCTTATTCTCCcgaccatggctgtagactgttaAATACTTCAAATactggtctatat |
| 175 | engineered | cattatgagctgataagaaatgactTCAACCAAATCcgaccatggctgtagactgttaACAATATCCCTT |
| | Perf ctrl | agtcattcttatcattcagacacattagtatgttaagTATATCAAATTcgaccatggctgtagactgttaATATTC |
| | | TCTCCTcttaacaactaatgtttagtagtcaatacagattatgTACACATAATTcgaccatggctgtagactgtta |
| | | ATAATCCTATTTcataatcgtattgattctttcactatatacttcaaggTATTCTCTTCTcgaccatggctgta |
| | | gactgttaACACAACACACTccttgaatatatagttcatgccctgtttattgacatgTACTCATATTTcgac |
| | | catggctgtagactgttaATTTATCCTATTcatgtcataaacagttatacaa |
| SEQ ID NO: | miR-132 | TGATATACGACCAGTatagactacaaCGACCATGGCTGTAGACaTGTTAgaaactcaaga |
| 176 | Perf A | tACTGGTCTATATCAttatgagcTGATAAGAAATGACTcaacttacgtaCGACCATGGC |
| | | TGTAGACaTGTTAcaatatcacctcAGTCATTCTTATCActtagacaCATTAGTATGTTA |
| | | AGctcatccttatCGACCATGGCTGTAGACaTGTTAcactcaaatcatCTTAACAACTAAT |
| | | GtttagtagTCAATACAGATTATGactctatcaaaCGACCATGGCTGTAGACaTGTTAgca |
| | | acatcatcaCATAATCGTATTGAcatgcaatCTATATACTTCAAGGaagatgactaaCGACC |
| | | ATGGCTGTAGACaTGTTAcactgctagtaaCCTTGAATATATAGctgtagttCTGTTTAT |
| | | TGACATGtcaacatcaatCGACCATGGCTGTAGACaTGTTAcactttacctaaCATGTCAT |
| | | AAACAGtaactgaa |
| SEQ ID NO: | miR-132 | TGATATACGACCAGTatagactacaaCGACCATGGCTGTAGACgTGTTAgaaactcaag |
| 177 | Perf G | atACTGGTCTATATCAttatgagcTGATAAGAAATGACTcaacttacgtaCGACCATGGC |
| | | TGTAGACgTGTTAcaatatcacctcAGTCATTCTTATCActtagacaCATTAGTATGTTA |
| | | AGctcatccttatCGACCATGGCTGTAGACgTGTTAcactcaaatcatCTTAACAACTAAT |
| | | GtttagtagTCAATACAGATTATGactctatcaaaCGACCATGGCTGTAGACgTGTTAgc |
| | | atcatcatcaCATAATCGTATTGAcatgcaatCTATATACTTCAAGGaagatgactaaCGACC |
| | | ATGGCTGTAGACgTGTTAcactgctagtaaCCTTGAATATATAGctgaagttCTGTTTAT |
| | | TGACATGtcaacatcaatCGACCATGGCTGTAGACgTGTTAcactttacctaaCATGTCAT |
| | | AAACAGtaaccgaa |
| SEQ ID NO: | miR-132 | tgatatacgaccagtCACATACCTCCcgaccatggctgtagacctgttaATACTCTCACATactggtctat |
| 178 | Perf C | atcattatgagctgataagaaatgactACTATTCACAAcgaccatggctgtagacctgttaAATCCAAATT |
| | | CAagtcattcttatcattcagacacattagtatgttaagCCTCACTTCCCcgaccatggctgtagacctgttaAAC |
| | | ATTCCACCTcttaacaactaatgtttagtagtcaatacagattatgCACCCTATCAAcgaccatggctgtaga |
| | | cctgttaAACATACACACTcataatcgtattgattctttcactatatacttcaaggCCTTCTTCTATcgaccat |
| | | ggctgtagacctgttaACCTCACTAATAccttgaatatatagttcatgccctgtttattgacatgACACTCCCT |
| | | ATcgaccatggctgtagacctgttaCTTCTTCTATCAcatgtcataaacagttatacaa |
| SEQ ID NO: | miR-132 | TGATATACGACCAGTtacttcactctCGACCATGGCTGTAGACtTGTTActtacttcaactA |
| 179 | Perf T | CTGGTCTATATCAtcttgagcTGATAAGAAATGACTcatactaacttCGACCATGGCTG |
| | | TAGACtTGTTAAccttaTcattcAGTCATTCTTATCActtagacaCATTAGTATGTTAAG |
| | | ctcttactaatCGACCATGGCTGTAGACtTGTTActaacttaccctCTTAACAACTAATGttta |
| | | gtagTCAATACAGATTATGtcattccctatCGACCATGGCTGTAGACtTGTTAactcttacct |
| | | caCATAATCGTATTGAcatgcaatCTATATACTTCAAGGtttcatcccttCGACCATGGC |
| | | TGTAGACtTGTTAaatcccttcattCCTTGAATATATAGctttagttCTGTTTATTGACATG |
| | | tccactttaatCGACCATGGCTGTAGACtTGTTAccaactttcaatCATGTCATAAACAGtaa |
| | | ccgaa |
| SEQ ID NO: | miR-132 | TGATATACGACCAGTatagactacaaCGACCATGGCTCAGACTGTTAgaaactcaagatA |
| 180 | Imperf ctrl | CTGGTCTATATCAttatgagcTGATAAGAAATGACTcaacttacgtaCGACCATGGCTC |
| | | AGACTGTTAcaatatcacctcAGTCATTCTTATCActtagacaCATTAGTATGTTAAGctc |
| | | atccttatCGACCATGGCTCAGACTGTTAcactcaaatcatCTTAACAACTAATGtttagtag |
| | | TCAATACAGATTATGactctatcaaaCGACCATGGCTCAGACTGTTAgcaacatcatcaC |
| | | ATAATCGTATTGActtgcaatCTATATACTTCAAGGaagatcactaaCGACCATGGCTC |
| | | AGACTGTTAcactactaccaaCCTTGAATATATAGccgtagttCTGTTTATTGACATGtca |
| | | acatcaatCGACCATGGCTCAGACTGTTAcactttacctaaCATGTCATAAACAGtaacaga |
| | | a |
| SEQ ID NO: | miR-132 | TGATATACGACCAGTATAGACTACAACGACCATGGCTCAGACATGTTAGAA |
| 181 | Imperf A | ACTCAAGATACTGGTCTATATCATTATGAGCTGATAAGAAATGACTCAACT |
| | | TACGTACGACCATGGCTCAGACATGTTACAATATCACCTCAGTCATTCTTAT |
| | | CACTTAGACACATTAGTATGTTAAGCTCATCCTTATCGACCATGGCTCAGA |
| | | CATGTTACACTCAAATCATCTTAACAACTAATGTTTAGTAGTCAATACAGA |
| | | TTATGACTCTATCAAACGACCATGGCTCAGACATGTTAGCAACATCATCAC |
| | | ATAATCGTATTGACTTGCAATCTATATACTTCAAGGAAGATCACTAACGAC |
| | | CATGGCTCAGACATGTTACACTACTACCAACCTTGAATATATAGCCGTAGT |
| | | TCTGTTTATTGACATGTCAACATCAATCGACCATGGCTCAGACATGTTACAC |
| | | TTTACCTAACATGTCATAAACAGTAACAGAA |
| SEQ ID NO: | miR-132 | TGATATACGACCAGTatagactacaaCGACCATGGCTCAGACgTGTTAgaaactcaagat |
| 182 | Imperf G | ACTGGTCTATATCAttatgagcTGATAAGAAATGACTcaacttacgtaCGACCATGGCT |
| | | CAGACgTGTTAcaatatcacctcAGTCATTCTTATCActtagacaCATTAGTATGTTAAG |
| | | ctcatccttatCGACCATGGCTCAGACgTGTTAcactcaaatcatCTTAACAACTAATGtttag |
| | | tagTCAATACAGATTATGactctatcaaaCGACCATGGCTCAGACgTGTTAgcaacatcat |
| | | caCATAATCGTATTGActtgcaatCTATATACTTCAAGGaagatcactaaCGACCATGGC |
| | | TCAGACgTGTTAcactactaccaaCCTTGAATATATAGccgtagttCTGTTTATTGACAT |
| | | GtcaacatcaatCGACCATGGCTCAGACgTGTTAcactttacctaaCATGTCATAAACAGta |
| | | acagaa |
| SEQ ID NO: | miR-132 | TGATATACGACCAGTatagactacaaCGACCATGGCTCAGACcTGTTAgaaactcaagat |
| 183 | Imperf C | ACTGGTCTATATCAttatgagcTGATAAGAAATGACTcaacttacgtaCGACCATGGCT |
| | | CAGACCTGTTAcaatatcacctcAGTCATTCTTATCActtagacaCATTAGTATGTTAAG |
| | | ctcatccttatCGACCATGGCTCAGACCTGTTAcactcaaatcatCTTAACAACTAATGtttag |
| | | tagTCAATACAGATTATGactctatcaaaCGACCATGGCTCAGACCTGTTAgcaacatcatc |
| | | aCATAATCGTATTGActtgcaatCTATATACTTCAAGGaagatcactaaCGACCATGGC |
| | | TCAGACCTGTTAcactactaccaaCCTTGAATATATAGccgtagttCTGTTTATTGACAT |
| | | GtcaacatcaatCGACCATGGCTCAGACCTGTTAcactttacctaaCATGTCATAAACAGta |
| | | acagaa |
| SEQ ID NO: | miR-132 | TGATATACGACCAGTatagactacaaCGACCATGGCTCAGACtTGTTAgaaactcaagat |
| 184 | Imperf T | ACTGGTCTATATCAttatgagcTGATAAGAAATGACTcaacttacctaCGACCATGGCT |
| | | CAGACtTGTTAcaatatcacctcAGTCATTCTTATCActtagacaCATTAGTATGTTAAGc |
| | | ttacgcatacCGACCATGGCTCAGACtTGTTAcactctaatcctCTTAACAACTAATGtttagt |
| | | agTCAATACAGATTATGactctatcaaaCGACCATGGCTCAGACtTGTTAgcaacatcatca |
| | | CATAATCGTATTGActtgcaatCTATATACTTCAAGGatcctcacactCGACCATGGCT |
| | | CAGACtTGTTAcactacatccatCCTTGAATATATAGccgtagttCTGTTTATTGACATGt |
| | | caacatcaatCGACCATGGCTCAGACtTGTTAcactttacctaaCATGTCATAAACAGtaaca |
| | | gaa |
| SEQ ID NO: | miR-155 | TGATATACGACCAGTgcgcactacacAACCCCTATCACGATTAGCaATTAAgaaactc |
| 185 | Perf A | aagatACTGGTCTATATCAttatgagcTGATAAGAAATGACTactttaactacAACCCCTA |
| | | TCACGATTAGCaATTAAgaacacGacatCAGTCATTCTTATCAttcagacaCATTAGTA |
| | | TGTTAAGttaagcatcatAACCCCTATCACGATTAGCaATTAAcaacgaaacgatCTTAAC |
| | | AACTAATGtttagtagTCAATACAGATTATGacagcatctatAACCCCTATCACGATTA |
| | | GCaATTAAtacaccacgacgCATAATCGTATTGAttctttcaCTATATACTTCAAGGaccat |
| | | cactacAACCCCTATCACGATTAGCaATTAAcactactagcagCCTTGAATATATAGttc |
| | | atgccCTGTTTATTGACATGtcacatctcacAACCCCTATCACGATTAGCaATTAAcaca |
| | | tcacgcatCATGTCATAAACAGttatacaa |
| SEQ ID NO: | miR-155 | TGATATACGACCAGTtcatactacacAACCCCTATCACGATTAGCgATTAAgaaactca |
| 186 | Perf G | atatACTGGTCTATATCAttatgagcTGATAAGAAATGACTacaccgacataAACCCCTA |
| | | TCACGATTAGCgATTAAcctacagagattAGTCATTCTTATCAttcagacaCATTAGTAT |
| | | GTTAAGtgactcatcatAACCCCTATCACGATTAGCgATTAAattcagaacgatCTTAACA |
| | | ACTAATGtttagtagTCAATACAGATTATGacagcaactatAACCCCTATCACGATTAG |
| | | CgATTAAtacaccacgaatCATAATCGTATTGAttctttcaCTATATACTTCAAGGacatacct |
| | | atcAACCCCTATCACGATTAGCgATTAAcaccatgaccagCCTTGAATATATAGttcatg |
| | | ccCTGTTTATTGACATGtcacatctcacAACCCCTATCACGATTAGCgATTAAcactcca |
| | | ctcatCATGTCATAAACAGttatacaa |
| SEQ ID NO: | miR-155 | TGATATACGACCAGTgcgcactacacAACCCCTATCACGATTAGCcATTAAgaaactc |
| 187 | Perf C | aagatACTGGTCTATATCAttatgagcTGATAAGAAATGACTactttaactacAACCCCTA |
| | | TCACGATTAGCCATTAAgaacacGacatCAGTCATTCTTATCAttcagacaCATTAGTA |
| | | TGTTAAGttaagcatcatAACCCCTATCACGATTAGCcATTAAcaacgaaacgatCTTAAC |
| | | AACTAATGtttagtagTCAATACAGATTATGacagcatctatAACCCCTATCACGATTA |
| | | GCcATTAAtacaccacgacgCATAATCGTATTGAttctttcaCTATATACTTCAAGGaccat |
| | | cactacAACCCCTATCACGATTAGCCATTAAcactactagcagCCTTGAATATATAGttc |
| | | atgccCTGTTTATTGACATGtcacatctcacAACCCCTATCACGATTAGCcATTAAcaca |
| | | tcacgcatCATGTCATAAACAGttatacaa |
| SEQ ID NO: | miR-155 | TGATATACGACCAGTgcgcactacacAACCCCTATCACGATTAGCtATTAAgaaactca |
| 188 | Perf T | agatACTGGTCTATATCAttatgagcTGATAAGAAATGACTactttaactacAACCCCTAT |
| | | CACGATTAGCtATTAAgaacacGacatCAGTCATTCTTATCAttcagacaCATTAGTAT |
| | | GTTAAGttaccgatcatAACCCCTATCACGATTAGCtATTAActtacaaacgatCTTAACA |
| | | ACTAATGtttagtagTCAATACAGATTATGacatcacgcatAACCCCTATCACGATTAG |
| | | CtATTAAtacaccacgacgCATAATCGTATTGAttctttcaCTATATACTTCAAGGactctatc |
| | | tacAACCCCTATCACGATTAGCtATTAAcacttcacgcctCCTTGAATATATAGttcatgc |
| | | cCTGTTTATTGACATGtcacatctcacAACCCCTATCACGATTAGCtATTAAcacatcac |
| | | gcatCATGTCATAAACAGttatacaa |
| SEQ ID NO: | miR-155 | TGATATACGACCAGTgcgcactacacAACCCCTATCACGTAAGCATTAAgaaactcaa |
| 189 | Imperf ctrl | gatACTGGTCTATATCAttatgagcTGATAAGAAATGACTatccgaactacAACCCCTAT |
| | | CACGTAAGCATTAAtcatccaacatgAGTCATTCTTATCAttcagacaCATTAGTATGTT |
| | | AAGtgactcatcatAACCCCTATCACGTAAGCATTAActactcaacgatCTTAACAACTA |
| | | ATGtttagtagTCAATACAGATTATGacagcatctatAACCCCTATCACGTAAGCATTA |
| | | AtacaccacgacgCATAATCGTATTGAttctttcaCTATATACTTCAAGGaccatcactacAAC |
| | | CCCTATCACGTAAGCATTAAcactactagcagCCTTGAATATATAGttcatgccCTGTTT |
| | | ATTGACATGtcacatctcacAACCCCTATCACGTAAGCATTAAcacatcacgcatCATGT |
| | | CATAAACAGttatacaa |
| SEQ ID NO: | miR-155 | TGATATACGACCAGTgcgcactacacAACCCCTATCACGTAAGCaATTAAgaaactcaa |
| 190 | Imperf A | gatACTGGTCTATATCAttatgagcTGATAAGAAATGACTatccgaactacAACCCCTAT |
| | | CACGTAAGCaATTAAtcatccaacatgAGTCATTCTTATCAttcagacaCATTAGTATGT |
| | | TAAGtgactcatcatAACCCCTATCACGTAAGCaATTAActactcaacgatCTTAACAACT |
| | | AATGtttagtagTCAATACAGATTATGacagcatctatAACCCCTATCACGTAAGCaATT |
| | | AAtacaccacgacgCATAATCGTATTGAttctttcaCTATATACTTCAAGGaccatcactacAA |
| | | CCCCTATCACGTAAGCaATTAAcactactagcagCCTTGAATATATAGttcatgccCTGT |
| | | TTATTGACATGtcacatctcacAACCCCTATCACGTAAGCaATTAAcacatcacgcatCAT |
| | | GTCATAAACAGttatacaa |
| SEQ ID NO: | miR-155 | TGATATACGACCAGTtcagactacacAACCCCTATCACGTAAGCgATTAAgaaactcaa |
| 191 | Imperf G | tatACTGGTCTATATCAttatgagcTGATAAGAAATGACTacaccgacataAACCCCTAT |
| | | CACGTAAGCgATTAAcctacagagattAGTCATTCTTATCAttcagacaCATTAGTATGT |
| | | TAAGtgactcagcatAACCCCTATCACGTAAGCgATTAAattcagaacaatCTTAACAACT |
| | | AATGtttagtagTCAATACAGATTATGacagcaactatAACCCCTATCACGTAAGCgAT |
| | | TAAtacaccacgaatCATAATCGTATTGAttctttcaCTATATACTTCAAGGacatacctatcA |
| | | ACCCCTATCACGTAAGCgATTAAcaccatcaccagCCTTGAATATATAGttcatgccCT |
| | | GTTTATTGACATGtcacatctcacAACCCCTATCACGTAAGCgATTAAcactccactcatC |
| | | ATGTCATAAACAGttatacaa |
| SEQ ID NO: | miR-155 | TGATATACGACCAGTgcgcactacacAACCCCTATCACGTAAGCcATTAAgaaactcaa |
| 192 | Imperf C | gatACTGGTCTATATCAttatgagcTGATAAGAAATGACTatccgaactacAACCCCTAT |
| | | CACGTAAGCcATTAAtcatccaacatgAGTCATTCTTATCAttcagacaCATTAGTATGT |
| | | TAAGtgactcatcatAACCCCTATCACGTAAGCcATTAActactcaacgatCTTAACAACT |
| | | AATGtttagtagTCAATACAGATTATGacagcatctatAACCCCTATCACGTAAGCcATT |
| | | AAtacaccacgacgCATAATCGTATTGAttctttcaCTATATACTTCAAGGaccatcactacAA |
| | | CCCCTATCACGTAAGCCATTAAcactactagcagCCTTGAATATATAGttcatgccCTGT |
| | | TTATTGACATGtcacatctcacAACCCCTATCACGTAAGCcATTAAcacatcacgcatCAT |
| | | GTCATAAACAGttatacaa |
| SEQ ID NO: | miR-155 | TGATATACGACCAGTgcgcactacacAACCCCTATCACGTAAGCtATTAAgaaactcaa |
| 193 | Imperf T | gatACTGGTCTATATCAttatgagcTGATAAGAAATGACTatccgaactacAACCCCTAT |
| | | CACGTAAGCtATTAAtcatccaacatgAGTCATTCTTATCAttcagacaCATTAGTATGT |
| | | TAAGtgactcatcatAACCCCTATCACGTAAGCtATTAActactcaacgatCTTAACAACT |
| | | AATGtttagtagTCAATACAGATTATGacaccatctatAACCCCTATCACGTAAGCtATT |
| | | AAtacaccacgacgCATAATCGTATTGAttctttcaCTATATACTTCAAGGaccatcacaacA |
| | | ACCCCTATCACGTAAGCtATTAAcacgaccacgagCCTTGAATATATAGttcatgccCT |
| | | GTTTATTGACATGtcacatctcacAACCCCTATCACGTAAGCtATTAAcacatcacgcatC |
| | | ATGTCATAAACAGttatacaa |
| SEQ ID NO: | miR-18a | TGATATACGACCAGTccgcactacacCTATCTGCACTAGATGCACCTTAtaaactcagaa |
| 194 | Perf ctrl | tACTGGTCTATATCAttatgagcTGATAAGAAATGACTagctcaagtccCTATCTGCAC |
| | | TAGATGCACCTTAataagtcctccgAGTCATTCTTATCAttcagacaCATTAGTATGTTA |
| | | AGtcactcacgatCTATCTGCACTAGATGCACCTTActactcaactatCTTAACAACTAAT |
| | | GtttagtagTCAATACAGATTATGacagcatagatCTATCTGCACTAGATGCACCTTAta |
| | | caccacgacgCATAATCGTATTGAttctttcaCTATATACTTCAAGGaccatcactacCTATC |
| | | TGCACTAGATGCACCTTAcactactaccagCCTTGAATATATAGttcatgccCTGTTTAT |
| | | TGACATGtcacatctcacCTATCTGCACTAGATGCACCTTAcacatcacgcatCATGTCAT |
| | | AAACAGttatacaa |
| SEQ ID NO: | miR-18a | TGATATACGACCAGTccgcactacacCTATCTGCACTAGATGCAaCCTTAtaaactcaga |
| 195 | Perf A | atACTGGTCTATATCAttatgagcTGATAAGAAATGACTagctcaagtccCTATCTGCAC |
| | | TAGATGCAaCCTTAataagtcctccgAGTCATTCTTATCAttcagacaCATTAGTATGTT |
| | | AAGtcactcacgatCTATCTGCACTAGATGCAaCCTTActactcaactatCTTAACAACTA |
| | | ATGtttagtagTCAATACAGATTATGacagcatagatCTATCTGCACTAGATGCAaCCTT |
| | | AtacaccacgacgCATAATCGTATTGAttctttcaCTATATACTTCAAGGaccatcactacCTA |
| | | TCTGCACTAGATGCAaCCTTAcactactaccagCCTTGAATATATAGttcatgccCTGTT |
| | | TATTGACATGtcacatctcacCTATCTGCACTAGATGCAaCCTTAcacatcacgcatCATG |
| | | TCATAAACAGttatacaa |
| SEQ ID NO: | miR-18a | TGATATACGACCAGTccgcactacacCTATCTGCACTAGATGCAgCCTTAtaaactcag |
| 196 | Perf G | aatACTGGTCTATATCAttatgagcTGATAAGAAATGACTagctcaagtccCTATCTGCA |
| | | CTAGATGCAgCCTTAataagtcctccgAGTCATTCTTATCAttcagacaCATTAGTATGT |
| | | TAAGtcactcacgatCTATCTGCACTAGATGCAgCCTTActactcaactatCTTAACAACT |
| | | AATGtttagtagTCAATACAGATTATGacagcatagatCTATCTGCACTAGATGCAgCC |
| | | TTAtacaccacgacgCATAATCGTATTGAttctttcaCTATATACTTCAAGGaccatcactacC |
| | | TATCTGCACTAGATGCAgCCTTAcactactaccagCCTTGAATATATAGttcatgccCTG |
| | | TTTATTGACATGtcacatctcacCTATCTGCACTAGATGCAgCCTTAcacatcacgcatCA |
| | | TGTCATAAACAGttatacaa |
| SEQ ID NO: | miR-18a | TGATATACGACCAGTccgcactacacCTATCTGCACTAGATGCAcCCTTAtaaactcaga |
| 197 | Perf C | atACTGGTCTATATCAttatgagcTGATAAGAAATGACTagctcaagtccCTATCTGCAC |
| | | TAGATGCAcCCTTAataagtcctccgAGTCATTCTTATCAttcagacaCATTAGTATGTT |
| | | AAGtcactcacgatCTATCTGCACTAGATGCAcCCTTActactcaactatCTTAACAACTA |
| | | ATGtttagtagTCAATACAGATTATGacagcatagatCTATCTGCACTAGATGCAcCCTT |
| | | AtacaccacgacgCATAATCGTATTGAttctttcaCTATATACTTCAAGGaccatcactacCTA |
| | | TCTGCACTAGATGCAcCCTTAcactactaccagCCTTGAATATATAGttcatgccCTGTT |
| | | TATTGACATGtcacatctcacCTATCTGCACTAGATGCAcCCTTAcacatcacgcatCATG |
| | | TCATAAACAGttatacaa |
| SEQ ID NO: | miR-18a | TGATATACGACCAGTccgcactacacCTATCTGCACTAGATGCAtCCTTAtaaactcaga |
| 198 | Perf T | atACTGGTCTATATCAttatgagcTGATAAGAAATGACTagctcaagtccCTATCTGCAC |
| | | TAGATGCAtCCTTAataagtcctccgAGTCATTCTTATCAttcagacaCATTAGTATGTT |
| | | AAGtcactcactatCTATCTGCACTAGATGCAtCCTTActactcaactatCTTAACAACTAA |
| | | TGtttagtagTCAATACAGATTATGacagcatagacCTATCTGCACTAGATGCAtCCTTA |
| | | tacaccacgacgCATAATCGTATTGAttctttcaCTATATACTTCAAGGaccatcactacCTAT |
| | | CTGCACTAGATGCAtCCTTAcactactaccagCCTTGAATATATAGttcatgccCTGTTT |
| | | ATTGACATGtcacatctcacCTATCTGCACTAGATGCAtCCTTAcacatcacgcatCATGT |
| | | CATAAACAGttatacaa |
| SEQ ID NO: | miR-18a | TGATATACGACCAGTccgcactacacCTATCTGCACTACTGCACCTTAtaaactcagaat |
| 199 | Imperf ctrl | ACTGGTCTATATCAttatgagcTGATAAGAAATGACTtcttcaagtccCTATCTGCACT |
| | | ACTGCACCTTAataagtcctcctAGTCATTCTTATCAttcagacaCATTAGTATGTTAAGt |
| | | gactcatcatCTATCTGCACTACTGCACCTTActactcaacgatCTTAACAACTAATGtttag |
| | | tagTCAATACAGATTATGacagcatctatCTATCTGCACTACTGCACCTTAtacaccacga |
| | | cgCATAATCGTATTGAttctttcaCTATATACTTCAAGGaccatcactacCTATCTGCACT |
| | | ACTGCACCTTAcactactagcagCCTTGAATATATAGttcatgccCTGTTTATTGACATG |
| | | tcacatctcacCTATCTGCACTACTGCACCTTAcacatcacgcatCATGTCATAAACAGttat |
| | | acaa |
| SEQ ID NO: | miR-18a | TGATATACGACCAGTccgcactacacCTATCTGCACTACTGCAaCCTTAtaaactcagaat |
| 200 | Imperf A | ACTGGTCTATATCAttatgagcTGATAAGAAATGACTtcttcaagtccCTATCTGCACT |
| | | ACTGCAaCCTTAataagtcctcctAGTCATTCTTATCAttcagacaCATTAGTATGTTAA |
| | | GtcactcatcatCTATCTGCACTACTGCAaCCTTActactcaactgtCTTAACAACTAATGtt |
| | | tagtagTCAATACAGATTATGacagcatagatCTATCTGCACTACTGCAaCCTTAtacacc |
| | | acgacgCATAATCGTATTGAttctttcaCTATATACTTCAAGGaccatcactacCTATCTGC |
| | | ACTACTGCAaCCTTAcactactaccagCCTTGAATATATAGttcatgccCTGTTTATTGA |
| | | CATGtcacatctcacCTATCTGCACTACTGCAaCCTTAcacatcacgcatCATGTCATAAA |
| | | CAGttatacaa |
| SEQ ID NO: | miR-18a | TGATATACGACCAGTccgcactacacCTATCTGCACTACTGCAgCCTTAtaaactcagaat |
| 201 | Imperf G | ACTGGTCTATATCAttatgagcTGATAAGAAATGACTtcttcaagtccCTATCTGCACT |
| | | ACTGCAgCCTTAataagtcctcctAGTCATTCTTATCAttcagacaCATTAGTATGTTAA |
| | | GtcactcatcatCTATCTGCACTACTGCAgCCTTActactcaactgtCTTAACAACTAATGtt |
| | | tagtagTCAATACAGATTATGacagcatagatCTATCTGCACTACTGCAgCCTTAtacacc |
| | | acgacgCATAATCGTATTGAttctttcaCTATATACTTCAAGGaccatcactacCTATCTGC |
| | | ACTACTGCAgCCTTAcactactaccagCCTTGAATATATAGttcatgccCTGTTTATTGA |
| | | CATGtcacatctcacCTATCTGCACTACTGCAgCCTTAcacatcacgcatCATGTCATAAA |
| | | CAGttatacaa |
| SEQ ID NO: | miR-18a | TGATATACGACCAGTccgcactacacCTATCTGCACTACTGCAcCCTTAtaaactcagaat |
| 202 | Imperf C | ACTGGTCTATATCAttatgagcTGATAAGAAATGACTtcttcaagtccCTATCTGCACT |
| | | ACTGCAcCCTTAataagtcctcctAGTCATTCTTATCAttcagacaCATTAGTATGTTAA |
| | | GtcactcatcatCTATCTGCACTACTGCAcCCTTActactcaactgtCTTAACAACTAATGtt |
| | | tagtagTCAATACAGATTATGacagcatagatCTATCTGCACTACTGCAcCCTTAtacacc |
| | | acgacgCATAATCGTATTGAttctttcaCTATATACTTCAAGGaccatcactacCTATCTGC |
| | | ACTACTGCAcCCTTAcactactaccagCCTTGAATATATAGttcatgccCTGTTTATTGA |
| | | CATGtcacatctcacCTATCTGCACTACTGCAcCCTTAcacatcacgcatCATGTCATAAA |
| | | CAGttatacaa |
| SEQ ID NO: | miR-18a | TGATATACGACCAGTccgcactacacCTATCTGCACTACTGCAtCCTTAtaaactcagaat |
| 203 | Imperf T | ACTGGTCTATATCAttatgagcTGATAAGAAATGACTtcttcaagtccCTATCTGCACT |
| | | ACTGCAtCCTTAataagtcctcctAGTCATTCTTATCAttcagacaCATTAGTATGTTAAG |
| | | tcactcatcatCTATCTGCACTACTGCAtCCTTActactcaactgtCTTAACAACTAATGttta |
| | | gtagTCAATACAGATTATGacagcatagatCTATCTGCACTACTGCAtCCTTAtacaccac |
| | | gacgCATAATCGTATTGAttctttcaCTATATACTTCAAGGaccatcactacCTATCTGCA |
| | | CTACTGCAtCCTTAcactactaccagCCTTGAATATATAGttcatgccCTGTTTATTGAC |
| | | ATGtcacatctcacCTATCTGCACTACTGCAtCCTTAcacatcacgcatCATGTCATAAAC |
| | | AGttatacaa |
| SEQ ID NO: | miR-17 | TGATATACGACCAGTcctcactactaCTACCTGCACTGTAAGCACTTTGacttcgccgatt |
| 204 | Perf ctrl | ACTGGTCTATATCAttatgagcTGATAAGAAATGACTatcctgactacCTACCTGCACT |
| | | GTAAGCACTTTGacatcctacctgAGTCATTCTTATCAttcagacaCATTAGTATGTTAA |
| | | GtcaagccacatCTACCTGCACTGTAAGCACTTTGctaagcgacactCTTAACAACTAAT |
| | | GtttagtagTCAATACAGATTATGacttccgatctCTACCTGCACTGTAAGCACTTTGtect |
| | | attacgtcCATAATCGTATTGAttctttcaCTATATACTTCAAGGaccatcacgaaCTACCTG |
| | | CACTGTAAGCACTTTGacctactaccatCCTTGAATATATAGttcatgccCTGTTTATTG |
| | | ACATGtcactccatacCTACCTGCACTGTAAGCACTTTGaccattcctcttCATGTCATAA |
| | | ACAGttatacaa |
| SEQ ID NO: | miR-17 | TGATATACGACCAGTcctcactactaCTACCTGCACTGTAAGCAaCTTTGacttcgccgat |
| 205 | Perf A | tACTGGTCTATATCAttatgagcTGATAAGAAATGACTatcctgactacCTACCTGCACT |
| | | GTAAGCAaCTTTGacatcctacctgAGTCATTCTTATCAttcagacaCATTAGTATGTTA |
| | | AGtcaagccacatCTACCTGCACTGTAAGCAaCTTTGctaagcgacactCTTAACAACTA |
| | | ATGtttagtagTCAATACAGATTATGacttccgatctCTACCTGCACTGTAAGCAaCTTT |
| | | GtcctattacgtcCATAATCGTATTGAttctttcaCTATATACTTCAAGGaccatcacgaaCTAC |
| | | CTGCACTGTAAGCAaCTTTGacctactaccatCCTTGAATATATAGttcatgccCTGTTT |
| | | ATTGACATGtcactccatacCTACCTGCACTGTAAGCAaCTTTGaccattcctcttCATGTC |
| | | ATAAACAGttatacaa |
| SEQ ID NO: | miR-17 | TGATATACGACCAGTcctcactactaCTACCTGCACTGTAAGCAgCTTTGacttcgccgat |
| 206 | Perf G | tACTGGTCTATATCAttatgagcTGATAAGAAATGACTatcctgactacCTACCTGCACT |
| | | GTAAGCAgCTTTGacatcctacctgAGTCATTCTTATCAttcagacaCATTAGTATGTTA |
| | | AGtcaagccacatCTACCTGCACTGTAAGCAgCTTTGctaagcgacactCTTAACAACTA |
| | | ATGtttagtagTCAATACAGATTATGacttccgatctCTACCTGCACTGTAAGCAgCTTT |
| | | GtcctattacgtcCATAATCGTATTGAttctttcaCTATATACTTCAAGGaccatcacgaaCTAC |
| | | CTGCACTGTAAGCAgCTTTGacctactaccatCCTTGAATATATAGttcatgccCTGTTT |
| | | ATTGACATGtcactccatacCTACCTGCACTGTAAGCAgCTTTGaccattcctcttCATGTC |
| | | ATAAACAGttatacaa |
| SEQ ID NO: | miR-17 | TGATATACGACCAGTcctcactactaCTACCTGCACTGTAAGCAcCTTTGacttcgccgat |
| 207 | Perf C | tACTGGTCTATATCAttatgagcTGATAAGAAATGACTatcctgactacCTACCTGCACT |
| | | GTAAGCAcCTTTGacatcctacctgAGTCATTCTTATCAttcagacaCATTAGTATGTTA |
| | | AGtcaagccacatCTACCTGCACTGTAAGCAcCTTTGctaagcgacactCTTAACAACTA |
| | | ATGtttagtagTCAATACAGATTATGacttccgatctCTACCTGCACTGTAAGCAcCTTT |
| | | GtcctattacgtcCATAATCGTATTGAttctttcaCTATATACTTCAAGGaccatcacgaaCTAC |
| | | CTGCACTGTAAGCAcCTTTGacctactaccatCCTTGAATATATAGttcatgccCTGTTT |
| | | ATTGACATGtcactccatacCTACCTGCACTGTAAGCAcCTTTGaccattcctcttCATGTC |
| | | ATAAACAGttatacaa |
| SEQ ID NO: | miR-17 | TGATATACGACCAGTcctcactactaCTACCTGCACTGTAAGCAtCTTTGacttcgccgat |
| 208 | Perf T | tACTGGTCTATATCAttatgagcTGATAAGAAATGACTatcctgactacCTACCTGCACT |
| | | GTAAGCAtCTTTGacatcctacctgAGTCATTCTTATCAttcagacaCATTAGTATGTTA |
| | | AGtcatgacacacCTACCTGCACTGTAAGCAtCTTTGacatccgacactCTTAACAACTAA |
| | | TGtttagtagTCAATACAGATTATGacttccgatctCTACCTGCACTGTAAGCAtCTTTGt |
| | | cctattacgtcCATAATCGTATTGAttctttcaCTATATACTTCAAGGaccatcacgaaCTACC |
| | | TGCACTGTAAGCAtCTTTGacctactaccatCCTTGAATATATAGttcatgccCTGTTTAT |
| | | TGACATGtcactccatacCTACCTGCACTGTAAGCAtCTTTGaccattcctcttCATGTCAT |
| | | AAACAGttatacaa |
| SEQ ID NO: | miR-17 | TGATATACGACCAGTccgcactacacCTACCTGCACTGATGCACTTTGtaaactcctgat |
| 209 | Imperf ctrl | ACTGGTCTATATCAttatgagcTGATAAGAAATGACTatccgaactacCTACCTGCACT |
| | | GATGCACTTTGccatccaacctgAGTCATTCTTATCAttcagacaCATTAGTATGTTAAG |
| | | tgactcctcatCTACCTGCACTGATGCACTTTGctactcaactatCTTAACAACTAATGtttag |
| | | tagTCAATACAGATTATGacatcatatatCTACCTGCACTGATGCACTTTGcccacctcga |
| | | cgCATAATCGTATTGAttctttcaCTATATACTTCAAGGaccatcactatCTACCTGCACT |
| | | GATGCACTTTGtcctactaccagCCTTGAATATATAGttcatgccCTGTTTATTGACATGt |
| | | cacatctcacCTACCTGCACTGATGCACTTTGcataacactcatCATGTCATAAACAGttata |
| | | caa |
| SEQ ID NO: | miR-17 | TGATATACGACCAGTccatacgacacCTACCTGCACTGATGCAaCTTTGctaactgatcat |
| 210 | Imperf A | ACTGGTCTATATCAttatgagcTGATAAGAAATGACTatccgaactacCTACCTGCACT |
| | | GATGCAaCTTTGacatgctgactgAGTCATTCTTATCAttcagacaCATTAGTATGTTAA |
| | | GtgactccacatCTACCTGCACTGATGCAaCTTTGctacgcctctatCTTAACAACTAATGt |
| | | ttagtagTCAATACAGATTATGactttcacgccCTACCTGCACTGATGCAaCTTTGactact |
| | | ttaccgCATAATCGTATTGAttctttcaCTATATACTTCAAGGagtctcacgctCTACCTGC |
| | | ACTGATGCAaCTTTGtactactaccagCCTTGAATATATAGttcatgccCTGTTTATTGA |
| | | CATGcgacaccactcCTACCTGCACTGATGCAaCTTTGctgaacactgatCATGTCATAA |
| | | ACAGttatacaa |
| SEQ ID NO: | miR-17 | TGATATACGACCAGTccatacgacacCTACCTGCACTGATGCAgCTTTGctaactgatcat |
| 211 | Imperf G | ACTGGTCTATATCAttatgagcTGATAAGAAATGACTatccgaactacCTACCTGCACT |
| | | GATGCAgCTTTGacatgctgactgAGTCATTCTTATCAttcagacaCATTAGTATGTTAA |
| | | GtgactccacatCTACCTGCACTGATGCAgCTTTGctacgcctctatCTTAACAACTAATGt |
| | | ttagtagTCAATACAGATTATGactttcacgccCTACCTGCACTGATGCAgCTTTGactact |
| | | ttaccgCATAATCGTATTGAttctttcaCTATATACTTCAAGGagtctcacgctCTACCTGC |
| | | ACTGATGCAgCTTTGtactactaccagCCTTGAATATATAGttcatgccCTGTTTATTGA |
| | | CATGcgacaccactcCTACCTGCACTGATGCAgCTTTGctgaacactgatCATGTCATAA |
| | | ACAGttatacaa |
| SEQ ID NO: | miR-17 | TGATATACGACCAGTccatacgacacCTACCTGCACTGATGCAcCTTTGctaactgatcat |
| 212 | Imperf C | ACTGGTCTATATCAttatgagcTGATAAGAAATGACTatccgaactacCTACCTGCACT |
| | | GATGCAcCTTTGacatgctgactgAGTCATTCTTATCAttcagacaCATTAGTATGTTAA |
| | | GtgactccacatCTACCTGCACTGATGCAcCTTTGctacgcctctatCTTAACAACTAATGt |
| | | ttagtagTCAATACAGATTATGactttcacgccCTACCTGCACTGATGCAcCTTTGactact |
| | | ttaccgCATAATCGTATTGAttctttcaCTATATACTTCAAGGagtctcacgctCTACCTGC |
| | | ACTGATGCAcCTTTGtactactaccagCCTTGAATATATAGttcatgccCTGTTTATTGA |
| | | CATGcgacaccactcCTACCTGCACTGATGCAcCTTTGctgaacactgatCATGTCATAA |
| | | ACAGttatacaa |
| SEQ ID NO: | miR-17 | TGATATACGACCAGTcctcactactaCTACCTGCACTGATGCAtCTTTGacttcgccgatt |
| 213 | Imperf T | ACTGGTCTATATCAttatgagcTGATAAGAAATGACTatcctgactacCTACCTGCACT |
| | | GATGCAtCTTTGacctgctacctgAGTCATTCTTATCAttcagacaCATTAGTATGTTAA |
| | | GtcaagccacatCTACCTGCACTGATGCAtCTTTGctaagcgacactCTTAACAACTAATG |
| | | tttagtagTCAATACAGATTATGacttccgatctCTACCTGCACTGATGCAtCTTTGtcctatt |
| | | acgtcCATAATCGTATTGAttctttcaCTATATACTTCAAGGaccttcacgaaCTACCTGCA |
| | | CTGATGCAtCTTTGacctactaccatCCTTGAATATATAGttcatgccCTGTTTATTGACA |
| | | TGtcactccatacCTACCTGCACTGATGCAtCTTTGaccattcctcttCATGTCATAAACAG |
| | | ttatacaa |
| SEQ ID NO: | Synthesized | gggtgatatacgaccagtTCTTATTCTCCcgaccatggctgtagactgttaAATACTTCAAATactggtct |
| 214 | miR-132 | atatcattatgagctgataagaaatgactTCAACCAAATCcgaccatggctgtagactgttaACAATATCCC |
| | engineered | TTagtcattcttatcattcagacacattagtatgttaagTATATCAAATTcgaccatggctgtagactgttaATAT |
| | Perf ctrl | TCTCTCCTcttaacaactaatgtttagtagtcaatacagattatgTACACATAATTcgaccatggctgtagact |
| | | gttaATAATCCTATTTcataatcgtattgattctttcactatatacttcaaggTATTCTCTTCTcgaccatggc |
| | | tgtagactgttaACACAACACACTccttgaatatatagttcatgccctgtttattgacatgTACTCATATTTc |
| | | gaccatggctgtagactgttaATTTATCCTATTcatgtcataaacagttatacaa |
| SEQ ID NO: | Synthesized | gggTGATATACGACCAGTatagactacaaCGACCATGGCTGTAGACaTGTTAgaaactc |
| 215 | miR-132 | aagatACTGGTCTATATCAttatgagcTGATAAGAAATGACTcaacttacgtaCGACCATG |
| | Perf A | GCTGTAGACaTGTTAcaatatcacctcAGTCATTCTTATCActtagacaCATTAGTATGT |
| | | TAAGctcatccttatCGACCATGGCTGTAGACaTGTTAcactcaaatcatCTTAACAACTA |
| | | ATGtttagtagTCAATACAGATTATGactctatcaaaCGACCATGGCTGTAGACaTGTTA |
| | | gcaacatcatcaCATAATCGTATTGAcatgcaatCTATATACTTCAAGGaagatgactaaCGA |
| | | CCATGGCTGTAGACaTGTTAcactgctagtaaCCTTGAATATATAGctgtagttCTGTTT |
| | | ATTGACATGtcaacatcaatCGACCATGGCTGTAGACaTGTTAcactttacctaaCATGTC |
| | | ATAAACAGtaactgaa |
| SEQ ID NO: | Synthesized | gggTGATATACGACCAGTatagactacaaCGACCATGGCTGTAGACgTGTTAgaaactc |
| 216 | miR-132 | aagatACTGGTCTATATCAttatgagcTGATAAGAAATGACTcaacttacgtaCGACCATG |
| | Perf G | GCTGTAGACgTGTTAcaatatcacctcAGTCATTCTTATCActtagacaCATTAGTATGT |
| | | TAAGctcatccttatCGACCATGGCTGTAGACgTGTTAcactcaaatcatCTTAACAACTA |
| | | ATGtttagtagTCAATACAGATTATGactctatcaaaCGACCATGGCTGTAGACgTGTTA |
| | | gcatcatcatcaCATAATCGTATTGAcatgcaatCTATATACTTCAAGGaagatgactaaCGA |
| | | CCATGGCTGTAGACgTGTTAcactgctagtaaCCTTGAATATATAGctgaagttCTGTTT |
| | | ATTGACATGtcaacatcaatCGACCATGGCTGTAGACgTGTTAcactttacctaaCATGTC |
| | | ATAAACAGtaaccgaa |
| SEQ ID NO: | Synthesized | gggtgatatacgaccagtCACATACCTCCcgaccatggctgtagacctgttaATACTCTCACATactggt |
| 217 | miR-132 | ctatatcattatgagctgataagaaatgactACTATTCACAAcgaccatggctgtagacctgttaAATCCAAA |
| | Perf C | TTCAagtcattcttatcattcagacacattagtatgttaagCCTCACTTCCCcgaccatggctgtagacctgttaA |
| | | ACATTCCACCTcttaacaactaatgtttagtagtcaatacagattatgCACCCTATCAAcgaccatggctgt |
| | | agacctgttaAACATACACACTcataatcgtattgattctttcactatatacttcaaggCCTTCTTCTATcga |
| | | ccatggctgtagacctgttaACCTCACTAATAccttgaatatatagttcatgccctgtttattgacatgACACTC |
| | | CCTATcgaccatggctgtagacctgttaCTTCTTCTATCAcatgtcataaacagttatacaa |
| SEQ ID NO: | Synthesized | gggTGATATACGACCAGTtacttcactctCGACCATGGCTGTAGACtTGTTActtacttcaa |
| 218 | miR-132 | ctACTGGTCTATATCAtcttgagcTGATAAGAAATGACTcatactaacttCGACCATGGC |
| | Perf T | TGTAGACtTGTTAAccttaTcattcAGTCATTCTTATCActtagacaCATTAGTATGTTA |
| | | AGctcttactaatCGACCATGGCTGTAGACtTGTTActaacttaccctCTTAACAACTAATG |
| | | tttagtagTCAATACAGATTATGtcattccctatCGACCATGGCTGTAGACtTGTTAactctta |
| | | cctcaCATAATCGTATTGAcatgcaatCTATATACTTCAAGGtttcatcccttCGACCATGG |
| | | CTGTAGACtTGTTAaatcccttcattCCTTGAATATATAGctttagttCTGTTTATTGACAT |
| | | GtccactttaatCGACCATGGCTGTAGACtTGTTAccaactttcaatCATGTCATAAACAGta |
| | | accgaa |
| SEQ ID NO: | Synthesized | gggTGATATACGACCAGTatagactacaaCGACCATGGCTCAGACTGTTAgaaactcaag |
| 219 | miR-132 | atACTGGTCTATATCAttatgagcTGATAAGAAATGACTcaacttacgtaCGACCATGGC |
| | Imperf ctrl | TCAGACTGTTAcaatatcacctcAGTCATTCTTATCActtagacaCATTAGTATGTTAAG |
| | | ctcatccttatCGACCATGGCTCAGACTGTTAcactcaaatcatCTTAACAACTAATGtttagt |
| | | agTCAATACAGATTATGactctatcaaaCGACCATGGCTCAGACTGTTAgcaacatcatcC |
| | | ATAATCGTATTGActtgcaatCTATATACTTCAAGGaagatcactaaCGACCATGGCTC |
| | | AGACTGTTAcactactaccaaCCTTGAATATATAGccgtagttCTGTTTATTGACATGtca |
| | | acatcaatCGACCATGGCTCAGACTGTTAcactttacctaaCATGTCATAAACAGtaacaga |
| | | a |
| SEQ ID NO: | Synthesized | gggTGATATACGACCAGTATAGACTACAACGACCATGGCTCAGACATGTTAG |
| 220 | miR-132 | AAACTCAAGATACTGGTCTATATCATTATGAGCTGATAAGAAATGACTCAA |
| | Imperf A | CTTACGTACGACCATGGCTCAGACATGTTACAATATCACCTCAGTCATTCTT |
| | | ATCACTTAGACACATTAGTATGTTAAGCTCATCCTTATCGACCATGGCTCAG |
| | | ACATGTTACACTCAAATCATCTTAACAACTAATGTTTAGTAGTCAATACAG |
| | | ATTATGACTCTATCAAACGACCATGGCTCAGACATGTTAGCAACATCATCA |
| | | CATAATCGTATTGACTTGCAATCTATATACTTCAAGGAAGATCACTAACGA |
| | | CCATGGCTCAGACATGTTACACTACTACCAACCTTGAATATATAGCCGTAG |
| | | TTCTGTTTATTGACATGTCAACATCAATCGACCATGGCTCAGACATGTTACA |
| | | CTTTACCTAACATGTCATAAACAGTAACAGAA |
| SEQ ID NO: | Synthesized | gggTGATATACGACCAGTatagactacaaCGACCATGGCTCAGACgTGTTAgaaactcaa |
| 221 | miR-132 | gatACTGGTCTATATCAttatgagcTGATAAGAAATGACTcaacttacgtaCGACCATGG |
| | Imperf G | CTCAGACgTGTTAcaatatcacctcAGTCATTCTTATCActtagacaCATTAGTATGTTA |
| | | AGctcatccttatCGACCATGGCTCAGACgTGTTAcactcaaatcatCTTAACAACTAATGt |
| | | ttagtagTCAATACAGATTATGactctatcaaaCGACCATGGCTCAGACgTGTTAgcaacat |
| | | catcaCATAATCGTATTGActtgcaatCTATATACTTCAAGGaagatcactaaCGACCATG |
| | | GCTCAGACgTGTTAcactactaccaaCCTTGAATATATAGccgtagttCTGTTTATTGAC |
| | | ATGtcaacatcaatCGACCATGGCTCAGACgTGTTAcactttacctaaCATGTCATAAACA |
| | | Gtaacagaa |
| SEQ ID NO: | Synthesized | gggTGATATACGACCAGTatagactacaaCGACCATGGCTCAGACcTGTTAgaaactcaa |
| 222 | miR-132 | gatACTGGTCTATATCAttatgagcTGATAAGAAATGACTcaacttacgtaCGACCATGG |
| | Imperf C | CTCAGACCTGTTAcaatatcacctcAGTCATTCTTATCActtagacaCATTAGTATGTTA |
| | | AGctcatccttatCGACCATGGCTCAGACCTGTTAcactcaaatcatCTTAACAACTAATGt |
| | | ttagtagTCAATACAGATTATGactctatcaaaCGACCATGGCTCAGACCTGTTAgcaacat |
| | | catcaCATAATCGTATTGActtgcaatCTATATACTTCAAGGaagatcactaaCGACCATG |
| | | GCTCAGACCTGTTAcactactaccaaCCTTGAATATATAGccgtagttCTGTTTATTGAC |
| | | ATGtcaacatcaatCGACCATGGCTCAGACCTGTTAcactttacctaaCATGTCATAAACA |
| | | Gtaacagaa |
| SEQ ID NO: | Synthesized | gggTGATATACGACCAGTatagactacaaCGACCATGGCTCAGACtTGTTAgaaactcaa |
| 223 | miR-132 | gatACTGGTCTATATCAttatgagcTGATAAGAAATGACTcaacttacctaCGACCATGG |
| | Imperf T | CTCAGACtTGTTAcaatatcacctcAGTCATTCTTATCActtagacaCATTAGTATGTTA |
| | | AGcttacgcatacCGACCATGGCTCAGACtTGTTAcactctaatcctCTTAACAACTAATGt |
| | | ttagtagTCAATACAGATTATGactctatcaaaCGACCATGGCTCAGACtTGTTAgcaacat |
| | | catcaCATAATCGTATTGActtgcaatCTATATACTTCAAGGatcctcacactCGACCATG |
| | | GCTCAGACtTGTTAcactacatccatCCTTGAATATATAGccgtagttCTGTTTATTGAC |
| | | ATGtcaacatcaatCGACCATGGCTCAGACtTGTTAcactttacctaaCATGTCATAAACA |
| | | Gtaacagaa |
| SEQ ID NO: | Synthesized | gggTGATATACGACCAGTgcgcactacacAACCCCTATCACGATTAGCaATTAAgaa |
| 224 | miR-155 | actcaagatACTGGTCTATATCAttatgagcTGATAAGAAATGACTactttaactacAACCCC |
| | Perf A | TATCACGATTAGCaATTAAgaacacGacatCAGTCATTCTTATCAttcagacaCATTAG |
| | | TATGTTAAGttaagcatcatAACCCCTATCACGATTAGCaATTAAcaacgaaacgatCTTA |
| | | ACAACTAATGtttagtagTCAATACAGATTATGacagcatctatAACCCCTATCACGAT |
| | | TAGCaATTAAtacaccacgacgCATAATCGTATTGAttctttcaCTATATACTTCAAGGac |
| | | catcactacAACCCCTATCACGATTAGCaATTAAcactactagcagCCTTGAATATATAG |
| | | ttcatgccCTGTTTATTGACATGtcacatctcacAACCCCTATCACGATTAGCaATTAAca |
| | | catcacgcatCATGTCATAAACAGttatacaa |
| SEQ ID NO: | Synthesized | gggTGATATACGACCAGTtcatactacacAACCCCTATCACGATTAGCgATTAAgaaa |
| 225 | miR-155 | ctcaatatACTGGTCTATATCAttatgagcTGATAAGAAATGACTacaccgacataAACCCC |
| | Perf G | TATCACGATTAGCgATTAAcctacagagattAGTCATTCTTATCAttcagacaCATTAGT |
| | | ATGTTAAGtgactcatcatAACCCCTATCACGATTAGCgATTAAattcagaacgatCTTAA |
| | | CAACTAATGtttagtagTCAATACAGATTATGacagcaactatAACCCCTATCACGATT |
| | | AGCgATTAAtacaccacgaatCATAATCGTATTGAttctttcaCTATATACTTCAAGGacat |
| | | acctatcAACCCCTATCACGATTAGCgATTAAcaccatgaccagCCTTGAATATATAGtt |
| | | catgccCTGTTTATTGACATGtcacatctcacAACCCCTATCACGATTAGCgATTAAcac |
| | | tccactcatCATGTCATAAACAGttatacaa |
| SEQ ID NO: | Synthesized | gggTGATATACGACCAGTgcgcactacacAACCCCTATCACGATTAGCcATTAAgaa |
| 226 | miR-155 | actcaagatACTGGTCTATATCAttatgagcTGATAAGAAATGACTactttaactacAACCCC |
| | Perf C | TATCACGATTAGCCATTAAgaacacGacatCAGTCATTCTTATCAttcagacaCATTAG |
| | | TATGTTAAGttaagcatcatAACCCCTATCACGATTAGCcATTAAcaacgaaacgatCTTA |
| | | ACAACTAATGtttagtagTCAATACAGATTATGacagcatctatAACCCCTATCACGAT |
| | | TAGCcATTAAtacaccacgacgCATAATCGTATTGAttctttcaCTATATACTTCAAGGac |
| | | catcactacAACCCCTATCACGATTAGCcATTAAcactactagcagCCTTGAATATATAG |
| | | ttcatgccCTGTTTATTGACATGtcacatctcacAACCCCTATCACGATTAGCcATTAAca |
| | | catcacgcatCATGTCATAAACAGttatacaa |
| SEQ ID NO: | Synthesized | gggTGATATACGACCAGTgcgcactacacAACCCCTATCACGATTAGCtATTAAgaaa |
| 227 | miR-155 | ctcaagatACTGGTCTATATCAttatgagcTGATAAGAAATGACTactttaactacAACCCC |
| | Perf T | TATCACGATTAGCtATTAAgaacacGacatCAGTCATTCTTATCAttcagacaCATTAG |
| | | TATGTTAAGttaccgatcatAACCCCTATCACGATTAGCtATTAActtacaaacgatCTTA |
| | | ACAACTAATGtttagtagTCAATACAGATTATGacatcacgcatAACCCCTATCACGAT |
| | | TAGCtATTAAtacaccacgacgCATAATCGTATTGAttctttcaCTATATACTTCAAGGac |
| | | tctatctacAACCCCTATCACGATTAGCtATTAAcacttcacgcctCCTTGAATATATAGtt |
| | | catgccCTGTTTATTGACATGtcacatctcacAACCCCTATCACGATTAGCtATTAAcac |
| | | atcacgcatCATGTCATAAACAGttatacaa |
| SEQ ID NO: | Synthesized | gggTGATATACGACCAGTgcgcactacacAACCCCTATCACGTAAGCATTAAgaaact |
| 228 | miR-155 | caagatACTGGTCTATATCAttatgagcTGATAAGAAATGACTatccgaactacAACCCCT |
| | Imperf ctrl | ATCACGTAAGCATTAAtcatccaacatgAGTCATTCTTATCAttcagacaCATTAGTAT |
| | | GTTAAGtgactcatcatAACCCCTATCACGTAAGCATTAActactcaacgatCTTAACAAC |
| | | TAATGtttagtagTCAATACAGATTATGacagcatctatAACCCCTATCACGTAAGCAT |
| | | TAAtacaccacgacgCATAATCGTATTGAttctttcaCTATATACTTCAAGGaccatcactacA |
| | | ACCCCTATCACGTAAGCATTAAcactactagcagCCTTGAATATATAGttcatgccCTG |
| | | TTTATTGACATGtcacatctcacAACCCCTATCACGTAAGCATTAAcacatcacgcatCAT |
| | | GTCATAAACAGttatacaa |
| SEQ ID NO: | Synthesized | gggTGATATACGACCAGTgcgcactacacAACCCCTATCACGTAAGCaATTAAgaaac |
| 229 | miR-155 | tcaagatACTGGTCTATATCAttatgagcTGATAAGAAATGACTatccgaactacAACCCCT |
| | Imperf A | ATCACGTAAGCaATTAAtcatccaacatgAGTCATTCTTATCAttcagacaCATTAGTAT |
| | | GTTAAGtgactcatcatAACCCCTATCACGTAAGCaATTAActactcaacgatCTTAACAA |
| | | CTAATGtttagtagTCAATACAGATTATGacagcatctatAACCCCTATCACGTAAGCa |
| | | ATTAAtacaccacgacgCATAATCGTATTGAttctttcaCTATATACTTCAAGGaccatcacta |
| | | CAACCCCTATCACGTAAGCaATTAAcactactagcagCCTTGAATATATAGttcatgccC |
| | | TGTTTATTGACATGtcacatctcacAACCCCTATCACGTAAGCaATTAAcacatcacgcat |
| | | CATGTCATAAACAGttatacaa |
| SEQ ID NO: | Synthesized | gggTGATATACGACCAGTtcagactacacAACCCCTATCACGTAAGCgATTAAgaaact |
| 230 | miR-155 | caatatACTGGTCTATATCAttatgagcTGATAAGAAATGACTacaccgacataAACCCCT |
| | Imperf G | ATCACGTAAGCgATTAAcctacagagattAGTCATTCTTATCAttcagacaCATTAGTAT |
| | | GTTAAGtgactcagcatAACCCCTATCACGTAAGCgATTAAattcagaacaatCTTAACA |
| | | ACTAATGtttagtagTCAATACAGATTATGacagcaactatAACCCCTATCACGTAAGC |
| | | gATTAAtacaccacgaatCATAATCGTATTGAttctttcaCTATATACTTCAAGGacataccta |
| | | tcAACCCCTATCACGTAAGCgATTAAcaccatcaccagCCTTGAATATATAGttcatgcc |
| | | CTGTTTATTGACATGtcacatctcacAACCCCTATCACGTAAGCgATTAAcactccactca |
| | | tCATGTCATAAACAGttatacaa |
| SEQ ID NO: | Synthesized | gggTGATATACGACCAGTgcgcactacacAACCCCTATCACGTAAGCcATTAAgaaac |
| 231 | miR-155 | tcaagatACTGGTCTATATCAttatgagcTGATAAGAAATGACTatccgaactacAACCCCT |
| | Imperf C | ATCACGTAAGCcATTAAtcatccaacatgAGTCATTCTTATCAttcagacaCATTAGTAT |
| | | GTTAAGtgactcatcatAACCCCTATCACGTAAGCcATTAActactcaacgatCTTAACAA |
| | | CTAATGtttagtagTCAATACAGATTATGacagcatctatAACCCCTATCACGTAAGCc |
| | | ATTAAtacaccacgacgCATAATCGTATTGAttctttcaCTATATACTTCAAGGaccatcacta |
| | | CAACCCCTATCACGTAAGCCATTAAcactactagcagCCTTGAATATATAGttcatgccC |
| | | TGTTTATTGACATGtcacatctcacAACCCCTATCACGTAAGCcATTAAcacatcacgcat |
| | | CATGTCATAAACAGttatacaa |
| SEQ ID NO: | Synthesized | gggTGATATACGACCAGTgcgcactacacAACCCCTATCACGTAAGCtATTAAgaaact |
| 232 | miR-155 | caagatACTGGTCTATATCAttatgagcTGATAAGAAATGACTatccgaactacAACCCCT |
| | Imperf T | ATCACGTAAGCtATTAAtcatccaacatgAGTCATTCTTATCAttcagacaCATTAGTAT |
| | | GTTAAGtgactcatcatAACCCCTATCACGTAAGCtATTAActactcaacgatCTTAACAA |
| | | CTAATGtttagtagTCAATACAGATTATGacaccatctatAACCCCTATCACGTAAGCtA |
| | | TTAAtacaccacgacgCATAATCGTATTGAttctttcaCTATATACTTCAAGGaccatcacaac |
| | | AACCCCTATCACGTAAGCtATTAAcacgaccacgagCCTTGAATATATAGttcatgccC |
| | | TGTTTATTGACATGtcacatctcacAACCCCTATCACGTAAGCtATTAAcacatcacgcat |
| | | CATGTCATAAACAGttatacaa |
| SEQ ID NO: | Synthesized | gggTGATATACGACCAGTccgcactacacCTATCTGCACTAGATGCACCTTAtaaactc |
| 233 | miR-18a | agaatACTGGTCTATATCAttatgagcTGATAAGAAATGACTagctcaagtccCTATCTGC |
| | Perf ctrl | ACTAGATGCACCTTAataagtcctccgAGTCATTCTTATCAttcagacaCATTAGTATGT |
| | | TAAGtcactcacgatCTATCTGCACTAGATGCACCTTActactcaactatCTTAACAACTA |
| | | ATGtttagtagTCAATACAGATTATGacagcatagatCTATCTGCACTAGATGCACCTT |
| | | AtacaccacgacgCATAATCGTATTGAttctttcaCTATATACTTCAAGGaccatcactacCTA |
| | | TCTGCACTAGATGCACCTTAcactactaccagCCTTGAATATATAGttcatgccCTGTTT |
| | | ATTGACATGtcacatctcacCTATCTGCACTAGATGCACCTTAcacatcacgcatCATGTC |
| | | ATAAACAGttatacaa |
| SEQ ID NO: | Synthesized | gggTGATATACGACCAGTccgcactacacCTATCTGCACTAGATGCAaCCTTAtaaact |
| 234 | miR-18a | cagaatACTGGTCTATATCAttatgagcTGATAAGAAATGACTagctcaagtccCTATCTG |
| | Perf A | CACTAGATGCAaCCTTAataagtcctccgAGTCATTCTTATCAttcagacaCATTAGTAT |
| | | GTTAAGtcactcacgatCTATCTGCACTAGATGCAaCCTTActactcaactatCTTAACAA |
| | | CTAATGtttagtagTCAATACAGATTATGacagcatagatCTATCTGCACTAGATGCAa |
| | | CCTTAtacaccacgacgCATAATCGTATTGAttctttcaCTATATACTTCAAGGaccatcacta |
| | | CCTATCTGCACTAGATGCAaCCTTAcactactaccagCCTTGAATATATAGttcatgccC |
| | | TGTTTATTGACATGtcacatctcacCTATCTGCACTAGATGCAaCCTTAcacatcacgcat |
| | | CATGTCATAAACAGttatacaa |
| SEQ ID NO: | Synthesized | gggTGATATACGACCAGTccgcactacacCTATCTGCACTAGATGCAgCCTTAtaaact |
| 235 | miR-18a | cagaatACTGGTCTATATCAttatgagcTGATAAGAAATGACTagctcaagtccCTATCTG |
| | Perf G | CACTAGATGCAgCCTTAataagtcctccgAGTCATTCTTATCAttcagacaCATTAGTAT |
| | | GTTAAGtcactcacgatCTATCTGCACTAGATGCAgCCTTActactcaactatCTTAACAA |
| | | CTAATGtttagtagTCAATACAGATTATGacagcatagatCTATCTGCACTAGATGCAg |
| | | CCTTAtacaccacgacgCATAATCGTATTGAttctttcaCTATATACTTCAAGGaccatcacta |
| | | cCTATCTGCACTAGATGCAgCCTTAcactactaccagCCTTGAATATATAGttcatgccC |
| | | TGTTTATTGACATGtcacatctcacCTATCTGCACTAGATGCAgCCTTAcacatcacgcat |
| | | CATGTCATAAACAGttatacaa |
| SEQ ID NO: | Synthesized | gggTGATATACGACCAGTccgcactacacCTATCTGCACTAGATGCAcCCTTAtaaact |
| 236 | miR-18a | cagaatACTGGTCTATATCAttatgagcTGATAAGAAATGACTagctcaagtccCTATCTG |
| | Perf C | CACTAGATGCAcCCTTAataagtcctccgAGTCATTCTTATCAttcagacaCATTAGTAT |
| | | GTTAAGtcactcacgatCTATCTGCACTAGATGCAcCCTTActactcaactatCTTAACAA |
| | | CTAATGtttagtagTCAATACAGATTATGacagcatagatCTATCTGCACTAGATGCAc |
| | | CCTTAtacaccacgacgCATAATCGTATTGAttctttcaCTATATACTTCAAGGaccatcacta |
| | | cCTATCTGCACTAGATGCAcCCTTAcactactaccagCCTTGAATATATAGttcatgccC |
| | | TGTTTATTGACATGtcacatctcacCTATCTGCACTAGATGCAcCCTTAcacatcacgcat |
| | | CATGTCATAAACAGttatacaa |
| SEQ ID NO: | Synthesized | gggTGATATACGACCAGTccgcactacacCTATCTGCACTAGATGCAtCCTTAtaaactc |
| 237 | miR-18a | agaatACTGGTCTATATCAttatgagcTGATAAGAAATGACTagctcaagtccCTATCTGC |
| | Perf T | ACTAGATGCAtCCTTAataagtcctccgAGTCATTCTTATCAttcagacaCATTAGTATG |
| | | TTAAGtcactcactatCTATCTGCACTAGATGCAtCCTTActactcaactatCTTAACAACT |
| | | AATGtttagtagTCAATACAGATTATGacagcatagacCTATCTGCACTAGATGCAtCC |
| | | TTAtacaccacgacgCATAATCGTATTGAttctttcaCTATATACTTCAAGGaccatcactacC |
| | | TATCTGCACTAGATGCAtCCTTAcactactaccagCCTTGAATATATAGttcatgccCTG |
| | | TTTATTGACATGtcacatctcacCTATCTGCACTAGATGCAtCCTTAcacatcacgcatCAT |
| | | GTCATAAACAGttatacaa |
| SEQ ID NO: | Synthesized | gggTGATATACGACCAGTccgcactacacCTATCTGCACTACTGCACCTTAtaaactcag |
| 238 | miR-18a | aatACTGGTCTATATCAttatgagcTGATAAGAAATGACTtcttcaagtccCTATCTGCAC |
| | Imperf ctrl | TACTGCACCTTAataagtcctcctAGTCATTCTTATCAttcagacaCATTAGTATGTTAA |
| | | GtgactcatcatCTATCTGCACTACTGCACCTTActactcaacgatCTTAACAACTAATGttt |
| | | agtagTCAATACAGATTATGacagcatctatCTATCTGCACTACTGCACCTTAtacaccac |
| | | gacgCATAATCGTATTGAttctttcaCTATATACTTCAAGGaccatcactacCTATCTGCA |
| | | CTACTGCACCTTAcactactagcagCCTTGAATATATAGttcatgccCTGTTTATTGACA |
| | | TGtcacatctcacCTATCTGCACTACTGCACCTTAcacatcacgcatCATGTCATAAACAG |
| | | ttatacaa |
| SEQ ID NO: | Synthesized | gggTGATATACGACCAGTccgcactacacCTATCTGCACTACTGCAaCCTTAtaaactca |
| 239 | miR-18a | gaatACTGGTCTATATCAttatgagcTGATAAGAAATGACTtcttcaagtccCTATCTGCA |
| | Imperf A | CTACTGCAaCCTTAataagtcctcctAGTCATTCTTATCAttcagacaCATTAGTATGTTA |
| | | AGtcactcatcatCTATCTGCACTACTGCAaCCTTActactcaactgtCTTAACAACTAATG |
| | | tttagtagTCAATACAGATTATGacagcatagatCTATCTGCACTACTGCAaCCTTAtacac |
| | | cacgacgCATAATCGTATTGAttctttcaCTATATACTTCAAGGaccatcactacCTATCTG |
| | | CACTACTGCAaCCTTAcactactaccagCCTTGAATATATAGttcatgccCTGTTTATTG |
| | | ACATGtcacatctcacCTATCTGCACTACTGCAaCCTTAcacatcacgcatCATGTCATAA |
| | | ACAGttatacaa |
| SEQ ID NO: | Synthesized | gggTGATATACGACCAGTccgcactacacCTATCTGCACTACTGCAgCCTTAtaaactca |
| 240 | miR-18a | gaatACTGGTCTATATCAttatgagcTGATAAGAAATGACTtcttcaagtccCTATCTGCA |
| | Imperf G | CTACTGCAgCCTTAataagtcctcctAGTCATTCTTATCAttcagacaCATTAGTATGTT |
| | | AAGtcactcatcatCTATCTGCACTACTGCAgCCTTActactcaactgtCTTAACAACTAAT |
| | | GtttagtagTCAATACAGATTATGacagcatagatCTATCTGCACTACTGCAgCCTTAtac |
| | | accacgacgCATAATCGTATTGAttctttcaCTATATACTTCAAGGaccatcactacCTATCT |
| | | GCACTACTGCAgCCTTAcactactaccagCCTTGAATATATAGttcatgccCTGTTTATT |
| | | GACATGtcacatctcacCTATCTGCACTACTGCAgCCTTAcacatcacgcatCATGTCATA |
| | | AACAGttatacaa |
| SEQ ID NO: | Synthesized | gggTGATATACGACCAGTccgcactacacCTATCTGCACTACTGCAcCCTTAtaaactca |
| 241 | miR-18a | gaatACTGGTCTATATCAttatgagcTGATAAGAAATGACTtcttcaagtccCTATCTGCA |
| | Imperf C | CTACTGCAcCCTTAataagtcctcctAGTCATTCTTATCAttcagacaCATTAGTATGTTA |
| | | AGtcactcatcatCTATCTGCACTACTGCAcCCTTActactcaactgtCTTAACAACTAATG |
| | | tttagtagTCAATACAGATTATGacagcatagatCTATCTGCACTACTGCAcCCTTAtacac |
| | | cacgacgCATAATCGTATTGAttctttcaCTATATACTTCAAGGaccatcactacCTATCTG |
| | | CACTACTGCAcCCTTAcactactaccagCCTTGAATATATAGttcatgccCTGTTTATTG |
| | | ACATGtcacatctcacCTATCTGCACTACTGCAcCCTTAcacatcacgcatCATGTCATAA |
| | | ACAGttatacaa |
| SEQ ID NO: | Synthesized | gggTGATATACGACCAGTccgcactacacCTATCTGCACTACTGCAtCCTTAtaaactca |
| 242 | miR-18a | gaatACTGGTCTATATCAttatgagcTGATAAGAAATGACTtcttcaagtccCTATCTGCA |
| | Imperf T | CTACTGCAtCCTTAataagtcctcctAGTCATTCTTATCAttcagacaCATTAGTATGTTA |
| | | AGtcactcatcatCTATCTGCACTACTGCAtCCTTActactcaactgtCTTAACAACTAATG |
| | | tttagtagTCAATACAGATTATGacagcatagatCTATCTGCACTACTGCAtCCTTAtacac |
| | | cacgacgCATAATCGTATTGAttctttcaCTATATACTTCAAGGaccatcactacCTATCTG |
| | | CACTACTGCAtCCTTAcactactaccagCCTTGAATATATAGttcatgccCTGTTTATTG |
| | | ACATGtcacatctcacCTATCTGCACTACTGCAtCCTTAcacatcacgcatCATGTCATAA |
| | | ACAGttatacaa |
| SEQ ID NO: | Synthesized | gggTGATATACGACCAGTcctcactactaCTACCTGCACTGTAAGCACTTTGacttcgcc |
| 243 | miR-17 | gattACTGGTCTATATCAttatgagcTGATAAGAAATGACTatcctgactacCTACCTGCA |
| | Perf ctrl | CTGTAAGCACTTTGacatcctacctgAGTCATTCTTATCAttcagacaCATTAGTATGTT |
| | | AAGtcaagccacatCTACCTGCACTGTAAGCACTTTGctaagcgacactCTTAACAACTA |
| | | ATGtttagtagTCAATACAGATTATGacttccgatctCTACCTGCACTGTAAGCACTTTG |
| | | tcctattacgtcCATAATCGTATTGAttctttcaCTATATACTTCAAGGaccatcacgaaCTACC |
| | | TGCACTGTAAGCACTTTGacctactaccatCCTTGAATATATAGttcatgccCTGTTTAT |
| | | TGACATGtcactccatacCTACCTGCACTGTAAGCACTTTGaccattcctcttCATGTCAT |
| | | AAACAGttatacaa |
| SEQ ID NO: | Synthesized | gggTGATATACGACCAGTcctcactactaCTACCTGCACTGTAAGCAaCTTTGacttcgc |
| 244 | miR-17 | cgattACTGGTCTATATCAttatgagcTGATAAGAAATGACTatcctgactacCTACCTGC |
| | Perf A | ACTGTAAGCAaCTTTGacatcctacctgAGTCATTCTTATCAttcagacaCATTAGTATG |
| | | TTAAGtcaagccacatCTACCTGCACTGTAAGCAaCTTTGctaagegacactCTTAACAA |
| | | CTAATGtttagtagTCAATACAGATTATGacttccgatctCTACCTGCACTGTAAGCAaC |
| | | TTTGtcctattacgtcCATAATCGTATTGAttctttcaCTATATACTTCAAGGaccatcacgaaC |
| | | TACCTGCACTGTAAGCAaCTTTGacctactaccatCCTTGAATATATAGttcatgccCTG |
| | | TTTATTGACATGtcactccatacCTACCTGCACTGTAAGCAaCTTTGaccattcctcttCAT |
| | | GTCATAAACAGttatacaa |
| SEQ ID NO: | Synthesized | gggTGATATACGACCAGTcctcactactaCTACCTGCACTGTAAGCAgCTTTGacttegc |
| 245 | miR-17 | cgattACTGGTCTATATCAttatgagcTGATAAGAAATGACTatcctgactacCTACCTGC |
| | Perf G | ACTGTAAGCAgCTTTGacatcctacctgAGTCATTCTTATCAttcagacaCATTAGTATG |
| | | TTAAGtcaagccacatCTACCTGCACTGTAAGCAgCTTTGctaagcgacactCTTAACAA |
| | | CTAATGtttagtagTCAATACAGATTATGacttccgatctCTACCTGCACTGTAAGCAgC |
| | | TTTGtcctattacgtcCATAATCGTATTGAttctttcaCTATATACTTCAAGGaccatcacgaaC |
| | | TACCTGCACTGTAAGCAgCTTTGacctactaccatCCTTGAATATATAGttcatgccCTG |
| | | TTTATTGACATGtcactccatacCTACCTGCACTGTAAGCAgCTTTGaccattcctcttCAT |
| | | GTCATAAACAGttatacaa |
| SEQ ID NO: | Synthesized | gggTGATATACGACCAGTcctcactactaCTACCTGCACTGTAAGCAcCTTTGacttcgc |
| 246 | miR-17 | cgattACTGGTCTATATCAttatgagcTGATAAGAAATGACTatcctgactacCTACCTGC |
| | Perf C | ACTGTAAGCAcCTTTGacatcctacctgAGTCATTCTTATCAttcagacaCATTAGTATG |
| | | TTAAGtcaagccacatCTACCTGCACTGTAAGCAcCTTTGctaagcgacactCTTAACAA |
| | | CTAATGtttagtagTCAATACAGATTATGacttccgatctCTACCTGCACTGTAAGCAcC |
| | | TTTGtcctattacgtcCATAATCGTATTGAttctttcaCTATATACTTCAAGGaccatcacgaaC |
| | | TACCTGCACTGTAAGCAcCTTTGacctactaccatCCTTGAATATATAGttcatgccCTG |
| | | TTTATTGACATGtcactccatacCTACCTGCACTGTAAGCAcCTTTGaccattcctcttCAT |
| | | GTCATAAACAGttatacaa |
| SEQ ID NO: | Synthesized | gggTGATATACGACCAGTcctcactactaCTACCTGCACTGTAAGCAtCTTTGacttcgc |
| 247 | miR-17 | cgattACTGGTCTATATCAttatgagcTGATAAGAAATGACTatcctgactacCTACCTGC |
| | Perf T | ACTGTAAGCAtCTTTGacatcctacctgAGTCATTCTTATCAttcagacaCATTAGTATG |
| | | TTAAGtcatgacacacCTACCTGCACTGTAAGCAtCTTTGacatccgacactCTTAACAAC |
| | | TAATGtttagtagTCAATACAGATTATGacttccgatctCTACCTGCACTGTAAGCAtCT |
| | | TTGtcctattacgtcCATAATCGTATTGAttctttcaCTATATACTTCAAGGaccatcacgaaCT |
| | | ACCTGCACTGTAAGCAtCTTTGacctactaccatCCTTGAATATATAGttcatgccCTGTT |
| | | TATTGACATGtcactccatacCTACCTGCACTGTAAGCAtCTTTGaccattcctcttCATGT |
| | | CATAAACAGttatacaa |
| SEQ ID NO: | Synthesized | gggTGATATACGACCAGTccgcactacacCTACCTGCACTGATGCACTTTGtaaactect |
| 248 | miR-17 | gatACTGGTCTATATCAttatgagcTGATAAGAAATGACTatccgaactacCTACCTGCA |
| | Imperf ctrl | CTGATGCACTTTGccatccaacctgAGTCATTCTTATCAttcagacaCATTAGTATGTTA |
| | | AGtgactcctcatCTACCTGCACTGATGCACTTTGctactcaactatCTTAACAACTAATGt |
| | | ttagtagTCAATACAGATTATGacatcatatatCTACCTGCACTGATGCACTTTGcccacct |
| | | cgacgCATAATCGTATTGAttctttcaCTATATACTTCAAGGaccatcactatCTACCTGCA |
| | | CTGATGCACTTTGtcctactaccagCCTTGAATATATAGttcatgccCTGTTTATTGACA |
| | | TGtcacatctcacCTACCTGCACTGATGCACTTTGcataacactcatCATGTCATAAACAG |
| | | ttatacaa |
| SEQ ID NO: | Synthesized | gggTGATATACGACCAGTccatacgacacCTACCTGCACTGATGCAaCTTTGctaactga |
| 249 | miR-17 | tcatACTGGTCTATATCAttatgagcTGATAAGAAATGACTatcegaactacCTACCTGCA |
| | Imperf A | CTGATGCAaCTTTGacatgctgactgAGTCATTCTTATCAttcagacaCATTAGTATGTT |
| | | AAGtgactccacatCTACCTGCACTGATGCAaCTTTGctacgcctctatCTTAACAACTAA |
| | | TGtttagtagTCAATACAGATTATGactttcacgccCTACCTGCACTGATGCAaCTTTGac |
| | | tactttaccgCATAATCGTATTGAttctttcaCTATATACTTCAAGGagtctcacgctCTACCT |
| | | GCACTGATGCAaCTTTGtactactaccagCCTTGAATATATAGttcatgccCTGTTTATT |
| | | GACATGcgacaccactcCTACCTGCACTGATGCAaCTTTGetgaacactgatCATGTCAT |
| | | AAACAGttatacaa |
| SEQ ID NO: | Synthesized | gggTGATATACGACCAGTccatacgacacCTACCTGCACTGATGCAgCTTTGctaactga |
| 250 | miR-17 | tcatACTGGTCTATATCAttatgagcTGATAAGAAATGACTatccgaactacCTACCTGCA |
| | Imperf G | CTGATGCAgCTTTGacatgctgactgAGTCATTCTTATCAttcagacaCATTAGTATGTT |
| | | AAGtgactccacatCTACCTGCACTGATGCAgCTTTGctacgcctctatCTTAACAACTAA |
| | | TGtttagtagTCAATACAGATTATGactttcacgccCTACCTGCACTGATGCAgCTTTGac |
| | | tactttaccgCATAATCGTATTGAttctttcaCTATATACTTCAAGGagtctcacgctCTACCT |
| | | GCACTGATGCAgCTTTGtactactaccagCCTTGAATATATAGttcatgccCTGTTTATT |
| | | GACATGcgacaccactcCTACCTGCACTGATGCAgCTTTGetgaacactgatCATGTCAT |
| | | AAACAGttatacaa |
| SEQ ID NO: | Synthesized | gggTGATATACGACCAGTccatacgacacCTACCTGCACTGATGCAcCTTTGctaactga |
| 251 | miR-17 | tcatACTGGTCTATATCAttatgagcTGATAAGAAATGACTatccgaactacCTACCTGCA |
| | Imperf C | CTGATGCAcCTTTGacatgctgactgAGTCATTCTTATCAttcagacaCATTAGTATGTT |
| | | AAGtgactccacatCTACCTGCACTGATGCAcCTTTGctacgcctctatCTTAACAACTAA |
| | | TGtttagtagTCAATACAGATTATGactttcacgccCTACCTGCACTGATGCAcCTTTGac |
| | | tactttaccgCATAATCGTATTGAttctttcaCTATATACTTCAAGGagtctcacgctCTACCT |
| | | GCACTGATGCAcCTTTGtactactaccagCCTTGAATATATAGttcatgccCTGTTTATT |
| | | GACATGcgacaccactcCTACCTGCACTGATGCAcCTTTGctgaacactgatCATGTCAT |
| | | AAACAGttatacaa |
| SEQ ID NO: | Synthesized | gggTGATATACGACCAGTcctcactactaCTACCTGCACTGATGCAtCTTTGacttcgccg |
| 252 | miR-17 | attACTGGTCTATATCAttatgagcTGATAAGAAATGACTatcctgactacCTACCTGCAC |
| | Imperf T | TGATGCAtCTTTGacctgctacctgAGTCATTCTTATCAttcagacaCATTAGTATGTTA |
| | | AGtcaagccacatCTACCTGCACTGATGCAtCTTTGctaagcgacactCTTAACAACTAAT |
| | | GtttagtagTCAATACAGATTATGacttccgatctCTACCTGCACTGATGCAtCTTTGtccta |
| | | ttacgtcCATAATCGTATTGAttctttcaCTATATACTTCAAGGaccttcacgaaCTACCTGC |
| | | ACTGATGCAtCTTTGacctactaccatCCTTGAATATATAGttcatgccCTGTTTATTGAC |
| | | ATGtcactccatacCTACCTGCACTGATGCAtCTTTGaccattcctcttCATGTCATAAACA |
| | | Gttatacaa |

TABLE 8
Engineered Nucleic Acid Sequences (RNA)

| Sequence ID | Description | Sequence |
|---|---|---|
| SEQ ID NO: | miR-132 | ugauauacgaccaguUCUUAUUCUCCcgaccauggcuguagacuguuaAAUACUUCAAAUac |
| 253 | engineered | uggucuauaucauuaugagcugauaagaaaugacuUCAACCAAAUCcgaccauggcuguagacuguua |
| | Perf ctrl | ACAAUAUCCCUUagucauucuuaucauucagacacauuaguauguuaagUAUAUCAAAUUcg |
| | | accauggcuguagacuguuaAUAUUCUCUCCUcuuaacaacuaauguuuaguagucaauacagauuau |
| | | gUACACAUAAUUcgaccauggcuguagacuguuaAUAAUCCUAUUUcauaaucguauugauu |
| | | cuuucacuauauacuucaaggUAUUCUCUUCUcgaccauggcuguagacuguuaACACAACAC |
| | | ACUccuugaauauauaguucaugcccuguuuauugacaugUACUCAUAUUUcgaccauggcuguaga |
| | | cuguuaAUUUAUCCUAUUcaugucauaaacaguuauacaa |
| SEQ ID NO: | miR-132 | UGAUAUACGACCAGUauagacuacaaCGACCAUGGCUGUAGACaUGUUAgaaacuc |
| 254 | Perf A | aagauACUGGUCUAUAUCAuuaugagcUGAUAAGAAAUGACUcaacuuacguaCGAC |
| | | CAUGGCUGUAGACaUGUUAcaauaucaccucAGUCAUUCUUAUCAcuuagacaCAU |
| | | UAGUAUGUUAAGcucauccuuauCGACCAUGGCUGUAGACaUGUUAcacucaaauca |
| | | uCUUAACAACUAAUGuuuaguagUCAAUACAGAUUAUGacucuaucaaaCGACCAU |
| | | GGCUGUAGACaUGUUAgcaacaucaucaCAUAAUCGUAUUGAcaugcaauCUAUAU |
| | | ACUUCAAGGaagaugacuaaCGACCAUGGCUGUAGACaUGUUAcacugcuaguaaCC |
| | | UUGAAUAUAUAGcuguaguuCUGUUUAUUGACAUGucaacaucaauCGACCAUGG |
| | | CUGUAGACaUGUUAcacuuuaccuaaCAUGUCAUAAACAGuaacugaa |
| SEQ ID NO: | miR-132 | UGAUAUACGACCAGUauagacuacaaCGACCAUGGCUGUAGACgUGUUAgaaacuc |
| 255 | Perf G | aagauACUGGUCUAUAUCAuuaugagcUGAUAAGAAAUGACUcaacuuacguaCGAC |
| | | CAUGGCUGUAGACgUGUUAcaauaucaccucAGUCAUUCUUAUCAcuuagacaCAU |
| | | UAGUAUGUUAAGcucauccuuauCGACCAUGGCUGUAGACgUGUUAcacucaaauca |
| | | uCUUAACAACUAAUGuuuaguagUCAAUACAGAUUAUGacucuaucaaaCGACCAU |
| | | GGCUGUAGACgUGUUAgcaucaucaucaCAUAAUCGUAUUGAcaugcaauCUAUAU |
| | | ACUUCAAGGaagaugacuaaCGACCAUGGCUGUAGACgUGUUAcacugcuaguaaCC |
| | | UUGAAUAUAUAGcugaaguuCUGUUUAUUGACAUGucaacaucaauCGACCAUGG |
| | | CUGUAGACgUGUUAcacuuuaccuaaCAUGUCAUAAACAGuaaccgaa |
| SEQ ID NO: | miR-132 | ugauauacgaccaguCACAUACCUCCcgaccauggcuguagaccuguuaAUACUCUCACAUac |
| 256 | Perf C | uggucuauaucauuaugagcugauaagaaaugacuACUAUUCACAAcgaccauggcuguagaccuguu |
| | | aAAUCCAAAUUCAagucauucuuaucauucagacacauuaguauguuaagCCUCACUUCCCcg |
| | | accauggcuguagaccuguuaAACAUUCCACCUcuuaacaacuaauguuuaguagucaauacagauua |
| | | ugCACCCUAUCAAcgaccauggcuguagaccuguuaAACAUACACACUcauaaucguauuga |
| | | uucuuucacuauauacuucaaggCCUUCUUCUAUcgaccauggcuguagaccuguuaACCUCACU |
| | | AAUAccuugaauauauaguucaugcccuguuuauugacaugACACUCCCUAUcgaccauggcugua |
| | | gaccuguuaCUUCUUCUAUCAcaugucauaaacaguuauacaa |
| SEQ ID NO: | miR-132 | UGAUAUACGACCAGUuacuucacucuCGACCAUGGCUGUAGACuUGUUAcuuacu |
| 257 | Perf U | ucaacuACUGGUCUAUAUCAucuugagcUGAUAAGAAAUGACUcauacuaacuuCGA |
| | | CCAUGGCUGUAGACuUGUUAAccuuaUcauucAGUCAUUCUUAUCAcuuagacaC |
| | | AUUAGUAUGUUAAGcucuuacuaauCGACCAUGGCUGUAGACuUGUUAcuaacuu |
| | | acccuCUUAACAACUAAUGuuuaguagUCAAUACAGAUUAUGucauucccuauCGAC |
| | | CAUGGCUGUAGACuUGUUAacucuuaccucaCAUAAUCGUAUUGAcaugcaauCUA |
| | | UAUACUUCAAGGuuucaucccuuCGACCAUGGCUGUAGACuUGUUAaaucccuucau |
| | | uCCUUGAAUAUAUAGcuuuaguuCUGUUUAUUGACAUGuccacuuuaauCGACCA |
| | | UGGCUGUAGACuUGUUAccaacuuucaauCAUGUCAUAAACAGuaaccgaa |
| SEQ ID NO: | miR-132 | UGAUAUACGACCAGUauagacuacaaCGACCAUGGCUCAGACUGUUAgaaacucaa |
| 258 | Imperf ctrl | gauACUGGUCUAUAUCAuuaugagcUGAUAAGAAAUGACUcaacuuacguaCGACC |
| | | AUGGCUCAGACUGUUAcaauaucaccucAGUCAUUCUUAUCAcuuagacaCAUUAG |
| | | UAUGUUAAGcucauccuuauCGACCAUGGCUCAGACUGUUAcacucaaaucauCUUA |
| | | ACAACUAAUGuuuaguagUCAAUACAGAUUAUGacucuaucaaaCGACCAUGGCU |
| | | CAGACUGUUAgcaacaucaucaCAUAAUCGUAUUGAcuugcaauCUAUAUACUUCA |
| | | AGGaagaucacuaaCGACCAUGGCUCAGACUGUUAcacuacuaccaaCCUUGAAUAU |
| | | AUAGccguaguuCUGUUUAUUGACAUGucaacaucaauCGACCAUGGCUCAGACU |
| | | GUUAcacuuuaccuaaCAUGUCAUAAACAGuaacagaa |
| SEQ ID NO: | miR-132 | UGAUAUACGACCAGUAUAGACUACAACGACCAUGGCUCAGACAUGUUAG |
| 259 | Imperf A | AAACUCAAGAUACUGGUCUAUAUCAUUAUGAGCUGAUAAGAAAUGACUC |
| | | AACUUACGUACGACCAUGGCUCAGACAUGUUACAAUAUCACCUCAGUCA |
| | | UUCUUAUCACUUAGACACAUUAGUAUGUUAAGCUCAUCCUUAUCGACCA |
| | | UGGCUCAGACAUGUUACACUCAAAUCAUCUUAACAACUAAUGUUUAGUA |
| | | GUCAAUACAGAUUAUGACUCUAUCAAACGACCAUGGCUCAGACAUGUUA |
| | | GCAACAUCAUCACAUAAUCGUAUUGACUUGCAAUCUAUAUACUUCAAGG |
| | | AAGAUCACUAACGACCAUGGCUCAGACAUGUUACACUACUACCAACCUU |
| | | GAAUAUAUAGCCGUAGUUCUGUUUAUUGACAUGUCAACAUCAAUCGACC |
| | | AUGGCUCAGACAUGUUACACUUUACCUAACAUGUCAUAAACAGUAACAG |
| | | AA |
| SEQ ID NO: | miR-132 | UGAUAUACGACCAGUauagacuacaaCGACCAUGGCUCAGACgUGUUAgaaacuca |
| 260 | Imperf G | agauACUGGUCUAUAUCAuuaugagcUGAUAAGAAAUGACUcaacuuacguaCGACC |
| | | AUGGCUCAGACgUGUUAcaauaucaccucAGUCAUUCUUAUCAcuuagacaCAUUA |
| | | GUAUGUUAAGcucauccuuauCGACCAUGGCUCAGACgUGUUAcacucaaaucauCU |
| | | UAACAACUAAUGuuuaguagUCAAUACAGAUUAUGacucuaucaaaCGACCAUGG |
| | | CUCAGACgUGUUAgcaacaucaucaCAUAAUCGUAUUGAcuugcaauCUAUAUACU |
| | | UCAAGGaagaucacuaaCGACCAUGGCUCAGACgUGUUAcacuacuaccaaCCUUGAA |
| | | UAUAUAGccguaguuCUGUUUAUUGACAUGucaacaucaauCGACCAUGGCUCAG |
| | | ACgUGUUAcacuuuaccuaaCAUGUCAUAAACAGuaacagaa |
| SEQ ID NO: | miR-132 | UGAUAUACGACCAGUauagacuacaaCGACCAUGGCUCAGACcUGUUAgaaacuca |
| 261 | Imperf C | agauACUGGUCUAUAUCAuuaugagcUGAUAAGAAAUGACUcaacuuacguaCGACC |
| | | AUGGCUCAGACCUGUUAcaauaucaccucAGUCAUUCUUAUCAcuuagacaCAUUA |
| | | GUAUGUUAAGcucauccuuauCGACCAUGGCUCAGACcUGUUAcacucaaaucauCU |
| | | UAACAACUAAUGuuuaguagUCAAUACAGAUUAUGacucuaucaaaCGACCAUGG |
| | | CUCAGACcUGUUAgcaacaucaucaCAUAAUCGUAUUGAcuugcaauCUAUAUACU |
| | | UCAAGGaagaucacuaaCGACCAUGGCUCAGACcUGUUAcacuacuaccaaCCUUGAA |
| | | UAUAUAGccguaguuCUGUUUAUUGACAUGucaacaucaauCGACCAUGGCUCAG |
| | | ACCUGUUAcacuuuaccuaaCAUGUCAUAAACAGuaacagaa |
| SEQ ID NO: | miR-132 | UGAUAUACGACCAGUauagacuacaaCGACCAUGGCUCAGACuUGUUAgaaacuca |
| 262 | Imperf U | agauACUGGUCUAUAUCAuuaugagcUGAUAAGAAAUGACUcaacuuaccuaCGACC |
| | | AUGGCUCAGACuUGUUAcaauaucaccucAGUCAUUCUUAUCAcuuagacaCAUUA |
| | | GUAUGUUAAGcuuacgcauacCGACCAUGGCUCAGACuUGUUAcacucuaauccuCU |
| | | UAACAACUAAUGuuuaguagUCAAUACAGAUUAUGacucuaucaaaCGACCAUGG |
| | | CUCAGACuUGUUAgcaacaucaucaCAUAAUCGUAUUGAcuugcaauCUAUAUACU |
| | | UCAAGGauccucacacuCGACCAUGGCUCAGACuUGUUAcacuacauccauCCUUGA |
| | | AUAUAUAGccguaguuCUGUUUAUUGACAUGucaacaucaauCGACCAUGGCUCA |
| | | GACuUGUUAcacuuuaccuaaCAUGUCAUAAACAGuaacagaa |
| SEQ ID NO: | miR-155 | UGAUAUACGACCAGUgcgcacuacacAACCCCUAUCACGAUUAGCaAUUAAgaaa |
| 263 | Perf A | cucaagauACUGGUCUAUAUCAuuaugagcUGAUAAGAAAUGACUacuuuaacuacAA |
| | | CCCCUAUCACGAUUAGCaAUUAAgaacacGacauCAGUCAUUCUUAUCAuucaga |
| | | caCAUUAGUAUGUUAAGuuaagcaucauAACCCCUAUCACGAUUAGCaAUUAAc |
| | | aacgaaacgauCUUAACAACUAAUGuuuaguagUCAAUACAGAUUAUGacagcaucuau |
| | | AACCCCUAUCACGAUUAGCaAUUAAuacaccacgacgCAUAAUCGUAUUGAuucu |
| | | uucaCUAUAUACUUCAAGGaccaucacuacAACCCCUAUCACGAUUAGCaAUUAA |
| | | cacuacuagcagCCUUGAAUAUAUAGuucaugccCUGUUUAUUGACAUGucacaucucac |
| | | AACCCCUAUCACGAUUAGCaAUUAAcacaucacgcauCAUGUCAUAAACAGuuau |
| | | acaa |
| SEQ ID NO: | miR-155 | UGAUAUACGACCAGUucauacuacacAACCCCUAUCACGAUUAGCgAUUAAgaaa |
| 264 | Perf G | cucaauauACUGGUCUAUAUCAuuaugagcUGAUAAGAAAUGACUacaccgacauaAA |
| | | CCCCUAUCACGAUUAGCgAUUAAccuacagagauuAGUCAUUCUUAUCAuucagac |
| | | aCAUUAGUAUGUUAAGugacucaucauAACCCCUAUCACGAUUAGCgAUUAAau |
| | | ucagaacgauCUUAACAACUAAUGuuuaguagUCAAUACAGAUUAUGacagcaacuauA |
| | | ACCCCUAUCACGAUUAGCgAUUAAuacaccacgaauCAUAAUCGUAUUGAuucuu |
| | | ucaCUAUAUACUUCAAGGacauaccuaucAACCCCUAUCACGAUUAGCgAUUAAc |
| | | accaugaccagCCUUGAAUAUAUAGuucaugccCUGUUUAUUGACAUGucacaucucac |
| | | AACCCCUAUCACGAUUAGCgAUUAAcacuccacucauCAUGUCAUAAACAGuuau |
| | | acaa |
| SEQ ID NO: | miR-155 | UGAUAUACGACCAGUgcgcacuacacAACCCCUAUCACGAUUAGCcAUUAAgaaa |
| 265 | Perf C | cucaagauACUGGUCUAUAUCAuuaugagcUGAUAAGAAAUGACUacuuuaacuacAA |
| | | CCCCUAUCACGAUUAGCcAUUAAgaacacGacauCAGUCAUUCUUAUCAuucaga |
| | | caCAUUAGUAUGUUAAGuuaagcaucauAACCCCUAUCACGAUUAGCcAUUAAc |
| | | aacgaaacgauCUUAACAACUAAUGuuuaguagUCAAUACAGAUUAUGacagcaucuau |
| | | AACCCCUAUCACGAUUAGCcAUUAAuacaccacgacgCAUAAUCGUAUUGAuucu |
| | | uucaCUAUAUACUUCAAGGaccaucacuacAACCCCUAUCACGAUUAGCcAUUAA |
| | | cacuacuagcagCCUUGAAUAUAUAGuucaugccCUGUUUAUUGACAUGucacaucucac |
| | | AACCCCUAUCACGAUUAGCcAUUAAcacaucacgcauCAUGUCAUAAACAGuuau |
| | | acaa |
| SEQ ID NO: | miR-155 | UGAUAUACGACCAGUgcgcacuacacAACCCCUAUCACGAUUAGCuAUUAAgaaa |
| 266 | Perf U | cucaagauACUGGUCUAUAUCAuuaugagcUGAUAAGAAAUGACUacuuuaacuacAA |
| | | CCCCUAUCACGAUUAGCuAUUAAgaacacGacauCAGUCAUUCUUAUCAuucaga |
| | | caCAUUAGUAUGUUAAGuuaccgaucauAACCCCUAUCACGAUUAGCuAUUAAc |
| | | uuacaaacgauCUUAACAACUAAUGuuuaguagUCAAUACAGAUUAUGacaucacgcau |
| | | AACCCCUAUCACGAUUAGCuAUUAAuacaccacgacgCAUAAUCGUAUUGAuucu |
| | | uucaCUAUAUACUUCAAGGacucuaucuacAACCCCUAUCACGAUUAGCuAUUA |
| | | AcacuucacgccuCCUUGAAUAUAUAGuucaugccCUGUUUAUUGACAUGucacaucuc |
| | | acAACCCCUAUCACGAUUAGCuAUUAAcacaucacgcauCAUGUCAUAAACAGuu |
| | | auacaa |
| SEQ ID NO: | miR-155 | UGAUAUACGACCAGUggcacuacacAACCCCUAUCACGUAAGCAUUAAgaaacu |
| 267 | Imperf ctrl | caagauACUGGUCUAUAUCAuuaugagcUGAUAAGAAAUGACUauccgaacuacAAC |
| | | CCCUAUCACGUAAGCAUUAAucauccaacaugAGUCAUUCUUAUCAuucagacaCA |
| | | UUAGUAUGUUAAGugacucaucauAACCCCUAUCACGUAAGCAUUAAcuacucaac |
| | | gauCUUAACAACUAAUGuuuaguagUCAAUACAGAUUAUGacagcaucuauAACCC |
| | | CUAUCACGUAAGCAUUAAuacaccacgacgCAUAAUCGUAUUGAuucuuucaCUAU |
| | | AUACUUCAAGGaccaucacuacAACCCCUAUCACGUAAGCAUUAAcacuacuagcag |
| | | CCUUGAAUAUAUAGuucaugccCUGUUUAUUGACAUGucacaucucacAACCCCU |
| | | AUCACGUAAGCAUUAAcacaucacgcauCAUGUCAUAAACAGuuauacaa |
| SEQ ID NO: | miR-155 | UGAUAUACGACCAGUgcgcacuacacAACCCCUAUCACGUAAGCaAUUAAgaaac |
| 268 | Imperf A | ucaagauACUGGUCUAUAUCAuuaugagcUGAUAAGAAAUGACUauccgaacuacAAC |
| | | CCCUAUCACGUAAGCaAUUAAucauccaacaugAGUCAUUCUUAUCAuucagacaC |
| | | AUUAGUAUGUUAAGugacucaucauAACCCCUAUCACGUAAGCaAUUAAcuacuc |
| | | aacgauCUUAACAACUAAUGuuuaguagUCAAUACAGAUUAUGacagcaucuauAAC |
| | | CCCUAUCACGUAAGCaAUUAAuacaccacgacgCAUAAUCGUAUUGAuucuuucaC |
| | | UAUAUACUUCAAGGaccaucacuacAACCCCUAUCACGUAAGCaAUUAAcacuacu |
| | | agcagCCUUGAAUAUAUAGuucaugccCUGUUUAUUGACAUGucacaucucacAACC |
| | | CCUAUCACGUAAGCaAUUAAcacaucacgcauCAUGUCAUAAACAGuuauacaa |
| SEQ ID NO: | miR-155 | UGAUAUACGACCAGUucagacuacacAACCCCUAUCACGUAAGCgAUUAAgaaac |
| 269 | Imperf G | ucaauauACUGGUCUAUAUCAuuaugagcUGAUAAGAAAUGACUacaccgacauaAAC |
| | | CCCUAUCACGUAAGCgAUUAAccuacagagauuAGUCAUUCUUAUCAuucagacaC |
| | | AUUAGUAUGUUAAGugacucagcauAACCCCUAUCACGUAAGCgAUUAAauucag |
| | | aacaauCUUAACAACUAAUGuuuaguagUCAAUACAGAUUAUGacagcaacuauAAC |
| | | CCCUAUCACGUAAGCgAUUAAuacaccacgaauCAUAAUCGUAUUGAuucuuucaC |
| | | UAUAUACUUCAAGGacauaccuaucAACCCCUAUCACGUAAGCgAUUAAcaccauc |
| | | accagCCUUGAAUAUAUAGuucaugccCUGUUUAUUGACAUGucacaucucacAACC |
| | | CCUAUCACGUAAGCgAUUAAcacuccacucauCAUGUCAUAAACAGuuauacaa |
| SEQ ID NO: | miR-155 | UGAUAUACGACCAGUgcgcacuacacAACCCCUAUCACGUAAGCcAUUAAgaaac |
| 270 | Imperf C | ucaagauACUGGUCUAUAUCAuuaugagcUGAUAAGAAAUGACUauccgaacuacAAC |
| | | CCCUAUCACGUAAGCcAUUAAucauccaacaugAGUCAUUCUUAUCAuucagacaC |
| | | AUUAGUAUGUUAAGugacucaucauAACCCCUAUCACGUAAGCcAUUAAcuacuc |
| | | aacgauCUUAACAACUAAUGuuuaguagUCAAUACAGAUUAUGacagcaucuauAAC |
| | | CCCUAUCACGUAAGCcAUUAAuacaccacgacgCAUAAUCGUAUUGAuucuuucaC |
| | | UAUAUACUUCAAGGaccaucacuacAACCCCUAUCACGUAAGCcAUUAAcacuacu |
| | | agcagCCUUGAAUAUAUAGuucaugccCUGUUUAUUGACAUGucacaucucacAACC |
| | | CCUAUCACGUAAGCcAUUAAcacaucacgcauCAUGUCAUAAACAGuuauacaa |
| SEQ ID NO: | miR-155 | UGAUAUACGACCAGUgcgcacuacacAACCCCUAUCACGUAAGCuAUUAAgaaac |
| 271 | Imperf U | ucaagauACUGGUCUAUAUCAuuaugagcUGAUAAGAAAUGACUauccgaacuacAAC |
| | | CCCUAUCACGUAAGCuAUUAAucauccaacaugAGUCAUUCUUAUCAuucagacaC |
| | | AUUAGUAUGUUAAGugacucaucauAACCCCUAUCACGUAAGCuAUUAAcuacuc |
| | | aacgauCUUAACAACUAAUGuuuaguagUCAAUACAGAUUAUGacaccaucuauAAC |
| | | CCCUAUCACGUAAGCuAUUAAuacaccacgacgCAUAAUCGUAUUGAuucuuucaC |
| | | UAUAUACUUCAAGGaccaucacaacAACCCCUAUCACGUAAGCuAUUAAcacgacc |
| | | acgagCCUUGAAUAUAUAGuucaugccCUGUUUAUUGACAUGucacaucucacAACC |
| | | CCUAUCACGUAAGCuAUUAAcacaucacgcauCAUGUCAUAAACAGuuauacaa |
| SEQ ID NO: | miR-18a | UGAUAUACGACCAGUccgcacuacacCUAUCUGCACUAGAUGCACCUUAuaaacu |
| 272 | Perf ctrl | cagaauACUGGUCUAUAUCAuuaugagcUGAUAAGAAAUGACUagcucaaguccCUA |
| | | UCUGCACUAGAUGCACCUUAauaaguccuccgAGUCAUUCUUAUCAuucagacaCA |
| | | UUAGUAUGUUAAGucacucacgauCUAUCUGCACUAGAUGCACCUUAcuacucaac |
| | | uauCUUAACAACUAAUGuuuaguagUCAAUACAGAUUAUGacagcauagauCUAUC |
| | | UGCACUAGAUGCACCUUAuacaccacgacgCAUAAUCGUAUUGAuucuuucaCUAU |
| | | AUACUUCAAGGaccaucacuacCUAUCUGCACUAGAUGCACCUUAcacuacuaccag |
| | | CCUUGAAUAUAUAGuucaugccCUGUUUAUUGACAUGucacaucucacCUAUCUG |
| | | CACUAGAUGCACCUUAcacaucacgcauCAUGUCAUAAACAGuuauacaa |
| SEQ ID NO: | miR-18a | UGAUAUACGACCAGUccgcacuacacCUAUCUGCACUAGAUGCAaCCUUAuaaac |
| 273 | Perf A | ucagaauACUGGUCUAUAUCAuuaugagcUGAUAAGAAAUGACUagcucaaguccCUA |
| | | UCUGCACUAGAUGCAaCCUUAauaaguccuccgAGUCAUUCUUAUCAuucagacaC |
| | | AUUAGUAUGUUAAGucacucacgauCUAUCUGCACUAGAUGCAaCCUUAcuacuc |
| | | aacuauCUUAACAACUAAUGuuuaguagUCAAUACAGAUUAUGacagcauagauCUA |
| | | UCUGCACUAGAUGCAaCCUUAuacaccacgacgCAUAAUCGUAUUGAuucuuucaC |
| | | UAUAUACUUCAAGGaccaucacuacCUAUCUGCACUAGAUGCAaCCUUAcacuacu |
| | | accagCCUUGAAUAUAUAGuucaugccCUGUUUAUUGACAUGucacaucucacCUAU |
| | | CUGCACUAGAUGCAaCCUUAcacaucacgcauCAUGUCAUAAACAGuuauacaa |
| SEQ ID NO: | miR-18a | UGAUAUACGACCAGUccgcacuacacCUAUCUGCACUAGAUGCAgCCUUAuaaac |
| 274 | Perf G | ucagaauACUGGUCUAUAUCAuuaugagcUGAUAAGAAAUGACUagcucaaguccCUA |
| | | UCUGCACUAGAUGCAgCCUUAauaaguccuccgAGUCAUUCUUAUCAuucagacaC |
| | | AUUAGUAUGUUAAGucacucacgauCUAUCUGCACUAGAUGCAgCCUUAcuacuc |
| | | aacuauCUUAACAACUAAUGuuuaguagUCAAUACAGAUUAUGacagcauagauCUA |
| | | UCUGCACUAGAUGCAgCCUUAuacaccacgacgCAUAAUCGUAUUGAuucuuucaC |
| | | UAUAUACUUCAAGGaccaucacuacCUAUCUGCACUAGAUGCAgCCUUAcacuacu |
| | | accagCCUUGAAUAUAUAGuucaugccCUGUUUAUUGACAUGucacaucucacCUAU |
| | | CUGCACUAGAUGCAgCCUUAcacaucacgcauCAUGUCAUAAACAGuuauacaa |
| SEQ ID NO: | miR-18a | UGAUAUACGACCAGUccgcacuacacCUAUCUGCACUAGAUGCAcCCUUAuaaac |
| 275 | Perf C | ucagaauACUGGUCUAUAUCAuuaugagcUGAUAAGAAAUGACUagcucaaguccCUA |
| | | UCUGCACUAGAUGCAcCCUUAauaaguccuccgAGUCAUUCUUAUCAuucagacaC |
| | | AUUAGUAUGUUAAGucacucacgauCUAUCUGCACUAGAUGCAcCCUUAcuacuc |
| | | aacuauCUUAACAACUAAUGuuuaguagUCAAUACAGAUUAUGacagcauagauCUA |
| | | UCUGCACUAGAUGCAcCCUUAuacaccacgacgCAUAAUCGUAUUGAuucuuucaC |
| | | UAUAUACUUCAAGGaccaucacuacCUAUCUGCACUAGAUGCAcCCUUAcacuacu |
| | | accagCCUUGAAUAUAUAGuucaugccCUGUUUAUUGACAUGucacaucucacCUAU |
| | | CUGCACUAGAUGCAcCCUUAcacaucacgcauCAUGUCAUAAACAGuuauacaa |
| SEQ ID NO: | miR-18a | UGAUAUACGACCAGUccgcacuacacCUAUCUGCACUAGAUGCAuCCUUAuaaac |
| 276 | Perf U | ucagaauACUGGUCUAUAUCAuuaugagcUGAUAAGAAAUGACUagcucaaguccCUA |
| | | UCUGCACUAGAUGCAuCCUUAauaaguccuccgAGUCAUUCUUAUCAuucagacaC |
| | | AUUAGUAUGUUAAGucacucacuauCUAUCUGCACUAGAUGCAuCCUUAcuacuc |
| | | aacuauCUUAACAACUAAUGuuuaguagUCAAUACAGAUUAUGacagcauagacCUA |
| | | UCUGCACUAGAUGCAuCCUUAuacaccacgacgCAUAAUCGUAUUGAuucuuucaC |
| | | UAUAUACUUCAAGGaccaucacuacCUAUCUGCACUAGAUGCAuCCUUAcacuacu |
| | | accagCCUUGAAUAUAUAGuucaugccCUGUUUAUUGACAUGucacaucucacCUAU |
| | | CUGCACUAGAUGCAuCCUUAcacaucacgcauCAUGUCAUAAACAGuuauacaa |
| SEQ ID NO: | miR-18a | UGAUAUACGACCAGUccgcacuacacCUAUCUGCACUACUGCACCUUAuaaacuca |
| 277 | Imperf ctrl | gaauACUGGUCUAUAUCAuuaugagcUGAUAAGAAAUGACUucuucaaguccCUAU |
| | | CUGCACUACUGCACCUUAauaaguccuccuAGUCAUUCUUAUCAuucagacaCAUU |
| | | AGUAUGUUAAGugacucaucauCUAUCUGCACUACUGCACCUUAcuacucaacgauC |
| | | UUAACAACUAAUGuuuaguagUCAAUACAGAUUAUGacagcaucuauCUAUCUGC |
| | | ACUACUGCACCUUAuacaccacgacgCAUAAUCGUAUUGAuucuuucaCUAUAUAC |
| | | UUCAAGGaccaucacuacCUAUCUGCACUACUGCACCUUAcacuacuagcagCCUUG |
| | | AAUAUAUAGuucaugccCUGUUUAUUGACAUGucacaucucacCUAUCUGCACUA |
| | | CUGCACCUUAcacaucacgcauCAUGUCAUAAACAGuuauacaa |
| SEQ ID NO: | miR-18a | UGAUAUACGACCAGUccgcacuacacCUAUCUGCACUACUGCAaCCUUAuaaacuc |
| 278 | Imperf A | agaauACUGGUCUAUAUCAuuaugagcUGAUAAGAAAUGACUucuucaaguccCUAU |
| | | CUGCACUACUGCAaCCUUAauaaguccuccuAGUCAUUCUUAUCAuucagacaCAU |
| | | UAGUAUGUUAAGucacucaucauCUAUCUGCACUACUGCAaCCUUAcuacucaacug |
| | | uCUUAACAACUAAUGuuuaguagUCAAUACAGAUUAUGacagcauagauCUAUCU |
| | | GCACUACUGCAaCCUUAuacaccacgacgCAUAAUCGUAUUGAuucuuucaCUAUA |
| | | UACUUCAAGGaccaucacuacCUAUCUGCACUACUGCAaCCUUAcacuacuaccagCC |
| | | UUGAAUAUAUAGuucaugccCUGUUUAUUGACAUGucacaucucacCUAUCUGCA |
| | | CUACUGCAaCCUUAcacaucacgcauCAUGUCAUAAACAGuuauacaa |
| SEQ ID NO: | miR-18a | UGAUAUACGACCAGUccgcacuacacCUAUCUGCACUACUGCAgCCUUAuaaacuc |
| 279 | Imperf G | agaauACUGGUCUAUAUCAuuaugagcUGAUAAGAAAUGACUucuucaaguccCUAU |
| | | CUGCACUACUGCAgCCUUAauaaguccuccuAGUCAUUCUUAUCAuucagacaCAU |
| | | UAGUAUGUUAAGucacucaucauCUAUCUGCACUACUGCAgCCUUAcuacucaacug |
| | | uCUUAACAACUAAUGuuuaguagUCAAUACAGAUUAUGacagcauagauCUAUCU |
| | | GCACUACUGCAgCCUUAuacaccacgacgCAUAAUCGUAUUGAuucuuucaCUAUA |
| | | UACUUCAAGGaccaucacuacCUAUCUGCACUACUGCAgCCUUAcacuacuaccagCC |
| | | UUGAAUAUAUAGuucaugccCUGUUUAUUGACAUGucacaucucacCUAUCUGCA |
| | | CUACUGCAgCCUUAcacaucacgcauCAUGUCAUAAACAGuuauacaa |
| SEQ ID NO: | miR-18a | UGAUAUACGACCAGUccgcacuacacCUAUCUGCACUACUGCAcCCUUAuaaacuc |
| 280 | Imperf C | agaauACUGGUCUAUAUCAuuaugagcUGAUAAGAAAUGACUucuucaaguccCUAU |
| | | CUGCACUACUGCAcCCUUAauaaguccuccuAGUCAUUCUUAUCAuucagacaCAU |
| | | UAGUAUGUUAAGucacucaucauCUAUCUGCACUACUGCAcCCUUAcuacucaacug |
| | | uCUUAACAACUAAUGuuuaguagUCAAUACAGAUUAUGacagcauagauCUAUCU |
| | | GCACUACUGCAcCCUUAuacaccacgacgCAUAAUCGUAUUGAuucuuucaCUAUA |
| | | UACUUCAAGGaccaucacuacCUAUCUGCACUACUGCAcCCUUAcacuacuaccagCC |
| | | UUGAAUAUAUAGuucaugccCUGUUUAUUGACAUGucacaucucacCUAUCUGCA |
| | | CUACUGCAcCCUUAcacaucacgcauCAUGUCAUAAACAGuuauacaa |
| SEQ ID NO: | miR-18a | UGAUAUACGACCAGUccgcacuacacCUAUCUGCACUACUGCAuCCUUAuaaacuc |
| 281 | Imperf U | agaauACUGGUCUAUAUCAuuaugagcUGAUAAGAAAUGACUucuucaaguccCUAU |
| | | CUGCACUACUGCAuCCUUAauaaguccuccuAGUCAUUCUUAUCAuucagacaCAU |
| | | UAGUAUGUUAAGucacucaucauCUAUCUGCACUACUGCAuCCUUAcuacucaacug |
| | | uCUUAACAACUAAUGuuuaguagUCAAUACAGAUUAUGacagcauagauCUAUCU |
| | | GCACUACUGCAuCCUUAuacaccacgacgCAUAAUCGUAUUGAuucuuucaCUAUA |
| | | UACUUCAAGGaccaucacuacCUAUCUGCACUACUGCAuCCUUAcacuacuaccagCC |
| | | UUGAAUAUAUAGuucaugccCUGUUUAUUGACAUGucacaucucacCUAUCUGCA |
| | | CUACUGCAuCCUUAcacaucacgcauCAUGUCAUAAACAGuuauacaa |
| SEQ ID NO: | miR-17 | UGAUAUACGACCAGUccucacuacuaCUACCUGCACUGUAAGCACUUUGacuucg |
| 282 | Perf ctrl | ccgauuACUGGUCUAUAUCAuuaugagcUGAUAAGAAAUGACUauccugacuacCUA |
| | | CCUGCACUGUAAGCACUUUGacauccuaccugAGUCAUUCUUAUCAuucagacaCA |
| | | UUAGUAUGUUAAGucaagccacauCUACCUGCACUGUAAGCACUUUGcuaagcgac |
| | | acuCUUAACAACUAAUGuuuaguagUCAAUACAGAUUAUGacuuccgaucuCUACC |
| | | UGCACUGUAAGCACUUUGuccuauuacgucCAUAAUCGUAUUGAuucuuucaCUA |
| | | UAUACUUCAAGGaccaucacgaaCUACCUGCACUGUAAGCACUUUGaccuacuacca |
| | | uCCUUGAAUAUAUAGuucaugccCUGUUUAUUGACAUGucacuccauacCUACCUG |
| | | CACUGUAAGCACUUUGaccauuccucuuCAUGUCAUAAACAGuuauacaa |
| SEQ ID NO: | miR-17 | UGAUAUACGACCAGUccucacuacuaCUACCUGCACUGUAAGCAaCUUUGacuuc |
| 283 | Perf A | gccgauuACUGGUCUAUAUCAuuaugagcUGAUAAGAAAUGACUauccugacuacCU |
| | | ACCUGCACUGUAAGCAaCUUUGacauccuaccugAGUCAUUCUUAUCAuucagaca |
| | | CAUUAGUAUGUUAAGucaagccacauCUACCUGCACUGUAAGCAaCUUUGcuaag |
| | | cgacacuCUUAACAACUAAUGuuuaguagUCAAUACAGAUUAUGacuuccgaucuCUA |
| | | CCUGCACUGUAAGCAaCUUUGuccuauuacgucCAUAAUCGUAUUGAuucuuucaC |
| | | UAUAUACUUCAAGGaccaucacgaaCUACCUGCACUGUAAGCAaCUUUGaccuacu |
| | | accauCCUUGAAUAUAUAGuucaugccCUGUUUAUUGACAUGucacuccauacCUAC |
| | | CUGCACUGUAAGCAaCUUUGaccauuccucuuCAUGUCAUAAACAGuuauacaa |
| SEQ ID NO: | miR-17 | UGAUAUACGACCAGUccucacuacuaCUACCUGCACUGUAAGCAgCUUUGacuuc |
| 284 | Perf G | gccgauuACUGGUCUAUAUCAuuaugagcUGAUAAGAAAUGACUauccugacuacCU |
| | | ACCUGCACUGUAAGCAgCUUUGacauccuaccugAGUCAUUCUUAUCAuucagaca |
| | | CAUUAGUAUGUUAAGucaagccacauCUACCUGCACUGUAAGCAgCUUUGcuaag |
| | | cgacacuCUUAACAACUAAUGuuuaguagUCAAUACAGAUUAUGacuuccgaucuCUA |
| | | CCUGCACUGUAAGCAgCUUUGuccuauuacgucCAUAAUCGUAUUGAuucuuucaC |
| | | UAUAUACUUCAAGGaccaucacgaaCUACCUGCACUGUAAGCAgCUUUGaccuacu |
| | | accauCCUUGAAUAUAUAGuucaugccCUGUUUAUUGACAUGucacuccauacCUAC |
| | | CUGCACUGUAAGCAgCUUUGaccauuccucuuCAUGUCAUAAACAGuuauacaa |
| SEQ ID NO: | miR-17 | UGAUAUACGACCAGUccucacuacuaCUACCUGCACUGUAAGCAcCUUUGacuuc |
| 285 | Perf C | gccgauuACUGGUCUAUAUCAuuaugagcUGAUAAGAAAUGACUauccugacuacCU |
| | | ACCUGCACUGUAAGCAcCUUUGacauccuaccugAGUCAUUCUUAUCAuucagaca |
| | | CAUUAGUAUGUUAAGucaagccacauCUACCUGCACUGUAAGCAcCUUUGcuaag |
| | | cgacacuCUUAACAACUAAUGuuuaguagUCAAUACAGAUUAUGacuuccgaucuCUA |
| | | CCUGCACUGUAAGCAcCUUUGuccuauuacgucCAUAAUCGUAUUGAuucuuucaC |
| | | UAUAUACUUCAAGGaccaucacgaaCUACCUGCACUGUAAGCAcCUUUGaccuacu |
| | | accauCCUUGAAUAUAUAGuucaugccCUGUUUAUUGACAUGucacuccauacCUAC |
| | | CUGCACUGUAAGCAcCUUUGaccauuccucuuCAUGUCAUAAACAGuuauacaa |
| SEQ ID NO: | miR-17 | UGAUAUACGACCAGUccucacuacuaCUACCUGCACUGUAAGCAuCUUUGacuuc |
| 286 | Perf U | gccgauuACUGGUCUAUAUCAuuaugagcUGAUAAGAAAUGACUauccugacuacCU |
| | | ACCUGCACUGUAAGCAuCUUUGacauccuaccugAGUCAUUCUUAUCAuucagaca |
| | | CAUUAGUAUGUUAAGucaugacacacCUACCUGCACUGUAAGCAuCUUUGacauc |
| | | cgacacuCUUAACAACUAAUGuuuaguagUCAAUACAGAUUAUGacuuccgaucuCUA |
| | | CCUGCACUGUAAGCAuCUUUGuccuauuacgucCAUAAUCGUAUUGAuucuuucaC |
| | | UAUAUACUUCAAGGaccaucacgaaCUACCUGCACUGUAAGCAuCUUUGaccuacu |
| | | accauCCUUGAAUAUAUAGuucaugccCUGUUUAUUGACAUGucacuccauacCUAC |
| | | CUGCACUGUAAGCAuCUUUGaccauuccucuuCAUGUCAUAAACAGuuauacaa |
| SEQ ID NO: | miR-17 | UGAUAUACGACCAGUccgcacuacacCUACCUGCACUGAUGCACUUUGuaaacucc |
| 287 | Imperf ctrl | ugauACUGGUCUAUAUCAuuaugagcUGAUAAGAAAUGACUauccgaacuacCUACC |
| | | UGCACUGAUGCACUUUGccauccaaccugAGUCAUUCUUAUCAuucagacaCAUUA |
| | | GUAUGUUAAGugacuccucauCUACCUGCACUGAUGCACUUUGcuacucaacuauCU |
| | | UAACAACUAAUGuuuaguagUCAAUACAGAUUAUGacaucauauauCUACCUGCA |
| | | CUGAUGCACUUUGcccaccucgacgCAUAAUCGUAUUGAuucuuucaCUAUAUACU |
| | | UCAAGGaccaucacuauCUACCUGCACUGAUGCACUUUGuccuacuaccagCCUUGA |
| | | AUAUAUAGuucaugccCUGUUUAUUGACAUGucacaucucacCUACCUGCACUGA |
| | | UGCACUUUGcauaacacucauCAUGUCAUAAACAGuuauacaa |
| SEQ ID NO: | miR-17 | UGAUAUACGACCAGUccauacgacacCUACCUGCACUGAUGCAaCUUUGcuaacug |
| 288 | Imperf A | aucauACUGGUCUAUAUCAuuaugagcUGAUAAGAAAUGACUauccgaacuacCUAC |
| | | CUGCACUGAUGCAaCUUUGacaugcugacugAGUCAUUCUUAUCAuucagacaCAU |
| | | UAGUAUGUUAAGugacuccacauCUACCUGCACUGAUGCAaCUUUGcuacgccucua |
| | | uCUUAACAACUAAUGuuuaguagUCAAUACAGAUUAUGacuuucacgccCUACCUG |
| | | CACUGAUGCAaCUUUGacuacuuuaccgCAUAAUCGUAUUGAuucuuucaCUAUAU |
| | | ACUUCAAGGagucucacgcuCUACCUGCACUGAUGCAaCUUUGuacuacuaccagCC |
| | | UUGAAUAUAUAGuucaugccCUGUUUAUUGACAUGegacaccacucCUACCUGCA |
| | | CUGAUGCAaCUUUGcugaacacugauCAUGUCAUAAACAGuuauacaa |
| SEQ ID NO: | miR-17 | UGAUAUACGACCAGUccauacgacacCUACCUGCACUGAUGCAgCUUUGcuaacug |
| 289 | Imperf G | aucauACUGGUCUAUAUCAuuaugagcUGAUAAGAAAUGACUauccgaacuacCUAC |
| | | CUGCACUGAUGCAgCUUUGacaugcugacugAGUCAUUCUUAUCAuucagacaCAU |
| | | UAGUAUGUUAAGugacuccacauCUACCUGCACUGAUGCAgCUUUGcuacgccucua |
| | | uCUUAACAACUAAUGuuuaguagUCAAUACAGAUUAUGacuuucacgccCUACCUG |
| | | CACUGAUGCAgCUUUGacuacuuuaccgCAUAAUCGUAUUGAuucuuucaCUAUAU |
| | | ACUUCAAGGagucucacgcuCUACCUGCACUGAUGCAgCUUUGuacuacuaccagCC |
| | | UUGAAUAUAUAGuucaugccCUGUUUAUUGACAUGcgacaccacucCUACCUGCA |
| | | CUGAUGCAgCUUUGcugaacacugauCAUGUCAUAAACAGuuauacaa |
| SEQ ID NO: | miR-17 | UGAUAUACGACCAGUccauacgacacCUACCUGCACUGAUGCAcCUUUGcuaacug |
| 290 | Imperf C | aucauACUGGUCUAUAUCAuuaugagcUGAUAAGAAAUGACUauccgaacuacCUAC |
| | | CUGCACUGAUGCAcCUUUGacaugcugacugAGUCAUUCUUAUCAuucagacaCAU |
| | | UAGUAUGUUAAGugacuccacauCUACCUGCACUGAUGCAcCUUUGcuacgccucua |
| | | uCUUAACAACUAAUGuuuaguagUCAAUACAGAUUAUGacuuucacgccCUACCUG |
| | | CACUGAUGCAcCUUUGacuacuuuaccgCAUAAUCGUAUUGAuucuuucaCUAUAU |
| | | ACUUCAAGGagucucacgcuCUACCUGCACUGAUGCAcCUUUGuacuacuaccagCC |
| | | UUGAAUAUAUAGuucaugccCUGUUUAUUGACAUGcgacaccacucCUACCUGCA |
| | | CUGAUGCAcCUUUGcugaacacugauCAUGUCAUAAACAGuuauacaa |
| SEQ ID NO: | miR-17 | UGAUAUACGACCAGUccucacuacuaCUACCUGCACUGAUGCAuCUUUGacuucgc |
| 291 | Imperf U | cgauuACUGGUCUAUAUCAuuaugagcUGAUAAGAAAUGACUauccugacuacCUAC |
| | | CUGCACUGAUGCAuCUUUGaccugcuaccugAGUCAUUCUUAUCAuucagacaCAU |
| | | UAGUAUGUUAAGucaagccacauCUACCUGCACUGAUGCAuCUUUGcuaagcgacac |
| | | uCUUAACAACUAAUGuuuaguagUCAAUACAGAUUAUGacuuccgaucuCUACCU |
| | | GCACUGAUGCAuCUUUGuccuauuacgucCAUAAUCGUAUUGAuucuuucaCUAUA |
| | | UACUUCAAGGaccuucacgaaCUACCUGCACUGAUGCAuCUUUGaccuacuaccauCC |
| | | UUGAAUAUAUAGuucaugccCUGUUUAUUGACAUGucacuccauacCUACCUGCA |
| | | CUGAUGCAuCUUUGaccauuccucuuCAUGUCAUAAACAGuuauacaa |
| SEQ ID NO: | Synthesized | gggugauauacgaccaguUCUUAUUCUCCcgaccauggcuguagacuguuaAAUACUUCAAA |
| 292 | miR-132 | UacuggucuauaucauuaugagcugauaagaaaugacuUCAACCAAAUCcgaccauggcuguagacug |
| | engineered | uuaACAAUAUCCCUUagucauucuuaucauucagacacauuaguauguuaagUAUAUCAAAU |
| | Perf ctrl | UcgaccauggcuguagacuguuaAUAUUCUCUCCUcuuaacaacuaauguuuaguagucaauacagau |
| | | uaugUACACAUAAUUcgaccauggcuguagacuguuaAUAAUCCUAUUUcauaaucguauug |
| | | auucuuucacuauauacuucaaggUAUUCUCUUCUcgaccauggcuguagacuguuaACACAAC |
| | | ACACUccuugaauauauaguucaugcccuguuuauugacaugUACUCAUAUUUcgaccauggcugu |
| | | agacuguuaAUUUAUCCUAUUcaugucauaaacaguuauacaa |
| SEQ ID NO: | Synthesized | gggUGAUAUACGACCAGUauagacuacaaCGACCAUGGCUGUAGACaUGUUAgaa |
| 293 | miR-132 | acucaagauACUGGUCUAUAUCAuuaugagcUGAUAAGAAAUGACUcaacuuacguaC |
| | Perf A | GACCAUGGCUGUAGACaUGUUAcaauaucaccucAGUCAUUCUUAUCAcuuagaca |
| | | CAUUAGUAUGUUAAGcucauccuuauCGACCAUGGCUGUAGACaUGUUAcacuca |
| | | aaucauCUUAACAACUAAUGuuuaguagUCAAUACAGAUUAUGacucuaucaaaCGA |
| | | CCAUGGCUGUAGACaUGUUAgcaacaucaucaCAUAAUCGUAUUGAcaugcaauCU |
| | | AUAUACUUCAAGGaagaugacuaaCGACCAUGGCUGUAGACaUGUUAcacugcuag |
| | | uaaCCUUGAAUAUAUAGcuguaguuCUGUUUAUUGACAUGucaacaucaauCGACC |
| | | AUGGCUGUAGACaUGUUAcacuuuaccuaaCAUGUCAUAAACAGuaacugaa |
| SEQ ID NO: | Synthesized | gggUGAUAUACGACCAGUauagacuacaaCGACCAUGGCUGUAGACgUGUUAgaa |
| 294 | miR-132 | acucaagauACUGGUCUAUAUCAuuaugagcUGAUAAGAAAUGACUcaacuuacguaC |
| | Perf G | GACCAUGGCUGUAGACgUGUUAcaauaucaccucAGUCAUUCUUAUCAcuuagaca |
| | | CAUUAGUAUGUUAAGcucauccuuauCGACCAUGGCUGUAGACgUGUUAcacuca |
| | | aaucauCUUAACAACUAAUGuuuaguagUCAAUACAGAUUAUGacucuaucaaaCGA |
| | | CCAUGGCUGUAGACgUGUUAgcaucaucaucaCAUAAUCGUAUUGAcaugcaauCU |
| | | AUAUACUUCAAGGaagaugacuaaCGACCAUGGCUGUAGACgUGUUAcacugcuag |
| | | uaaCCUUGAAUAUAUAGcugaaguuCUGUUUAUUGACAUGucaacaucaauCGACC |
| | | AUGGCUGUAGACgUGUUAcacuuuaccuaaCAUGUCAUAAACAGuaaccgaa |
| SEQ ID NO: | Synthesized | gggugauauacgaccaguCACAUACCUCCcgaccauggcuguagaccuguuaAUACUCUCACA |
| 295 | miR-132 | UacuggucuauaucauuaugagcugauaagaaaugacuACUAUUCACAAcgaccauggcuguagaccu |
| | Perf C | guuaAAUCCAAAUUCAagucauucuuaucauucagacacauuaguauguuaagCCUCACUUCC |
| | | CcgaccauggcuguagaccuguuaAACAUUCCACCUcuuaacaacuaauguuuaguagucaauacaga |
| | | uuaugCACCCUAUCAAcgaccauggcuguagaccuguuaAACAUACACACUcauaaucguau |
| | | ugauucuuucacuauauacuucaaggCCUUCUUCUAUcgaccauggcuguagaccuguuaACCUCA |
| | | CUAAUAccuugaauauauaguucaugcccuguuuauugacaugACACUCCCUAUcgaccauggcu |
| | | guagaccuguuaCUUCUUCUAUCAcaugucauaaacaguuauacaa |
| SEQ ID NO: | Synthesized | gggUGAUAUACGACCAGUuacuucacucuCGACCAUGGCUGUAGACuUGUUAcuu |
| 296 | miR-132 | acuucaacuACUGGUCUAUAUCAucuugagcUGAUAAGAAAUGACUcauacuaacuuC |
| | Perf U | GACCAUGGCUGUAGACuUGUUAAccuuaUcauucAGUCAUUCUUAUCAcuuagac |
| | | aCAUUAGUAUGUUAAGcucuuacuaauCGACCAUGGCUGUAGACuUGUUAcuaac |
| | | uuacccuCUUAACAACUAAUGuuuaguagUCAAUACAGAUUAUGucauucccuauCG |
| | | ACCAUGGCUGUAGACuUGUUAacucuuaccucaCAUAAUCGUAUUGAcaugcaauC |
| | | UAUAUACUUCAAGGuuucaucccuuCGACCAUGGCUGUAGACuUGUUAaaucccuu |
| | | cauuCCUUGAAUAUAUAGcuuuaguuCUGUUUAUUGACAUGuccacuuuaauCGAC |
| | | CAUGGCUGUAGACuUGUUAccaacuuucaauCAUGUCAUAAACAGuaaccgaa |
| SEQ ID NO: | Synthesized | gggUGAUAUACGACCAGUauagacuacaaCGACCAUGGCUCAGACUGUUAgaaacu |
| 297 | miR-132 | caagauACUGGUCUAUAUCAuuaugagcUGAUAAGAAAUGACUcaacuuacguaCGA |
| | Imperf ctrl | CCAUGGCUCAGACUGUUAcaauaucaccucAGUCAUUCUUAUCAcuuagacaCAUU |
| | | AGUAUGUUAAGcucauccuuauCGACCAUGGCUCAGACUGUUAcacucaaaucauCU |
| | | UAACAACUAAUGuuuaguagUCAAUACAGAUUAUGacucuaucaaaCGACCAUGG |
| | | CUCAGACUGUUAgcaacaucaucCAUAAUCGUAUUGAcuugcaauCUAUAUACUU |
| | | CAAGGaagaucacuaaCGACCAUGGCUCAGACUGUUAcacuacuaccaaCCUUGAAU |
| | | AUAUAGccguaguuCUGUUUAUUGACAUGucaacaucaauCGACCAUGGCUCAGA |
| | | CUGUUAcacuuuaccuaaCAUGUCAUAAACAGuaacagaa |
| SEQ ID NO: | Synthesized | gggUGAUAUACGACCAGUAUAGACUACAACGACCAUGGCUCAGACAUGUU |
| 298 | miR-132 | AGAAACUCAAGAUACUGGUCUAUAUCAUUAUGAGCUGAUAAGAAAUGAC |
| | Imperf A | UCAACUUACGUACGACCAUGGCUCAGACAUGUUACAAUAUCACCUCAGU |
| | | CAUUCUUAUCACUUAGACACAUUAGUAUGUUAAGCUCAUCCUUAUCGAC |
| | | CAUGGCUCAGACAUGUUACACUCAAAUCAUCUUAACAACUAAUGUUUAG |
| | | UAGUCAAUACAGAUUAUGACUCUAUCAAACGACCAUGGCUCAGACAUGU |
| | | UAGCAACAUCAUCACAUAAUCGUAUUGACUUGCAAUCUAUAUACUUCAA |
| | | GGAAGAUCACUAACGACCAUGGCUCAGACAUGUUACACUACUACCAACC |
| | | UUGAAUAUAUAGCCGUAGUUCUGUUUAUUGACAUGUCAACAUCAAUCGA |
| | | CCAUGGCUCAGACAUGUUACACUUUACCUAACAUGUCAUAAACAGUAAC |
| | | AGAA |
| SEQ ID NO: | Synthesized | gggUGAUAUACGACCAGUauagacuacaaCGACCAUGGCUCAGACgUGUUAgaaac |
| 299 | miR-132 | ucaagauACUGGUCUAUAUCAuuaugagcUGAUAAGAAAUGACUcaacuuacguaCGA |
| | Imperf G | CCAUGGCUCAGACgUGUUAcaauaucaccucAGUCAUUCUUAUCAcuuagacaCAU |
| | | UAGUAUGUUAAGcucauccuuauCGACCAUGGCUCAGACgUGUUAcacucaaaucau |
| | | CUUAACAACUAAUGuuuaguagUCAAUACAGAUUAUGacucuaucaaaCGACCAU |
| | | GGCUCAGACgUGUUAgcaacaucaucaCAUAAUCGUAUUGAcuugcaauCUAUAUA |
| | | CUUCAAGGaagaucacuaaCGACCAUGGCUCAGACgUGUUAcacuacuaccaaCCUUG |
| | | AAUAUAUAGccguaguuCUGUUUAUUGACAUGucaacaucaauCGACCAUGGCUC |
| | | AGACgUGUUAcacuuuaccuaaCAUGUCAUAAACAGuaacagaa |
| SEQ ID NO: | Synthesized | gggUGAUAUACGACCAGUauagacuacaaCGACCAUGGCUCAGACcUGUUAgaaac |
| 300 | miR-132 | ucaagauACUGGUCUAUAUCAuuaugagcUGAUAAGAAAUGACUcaacuuacguaCGA |
| | Imperf C | CCAUGGCUCAGACcUGUUAcaauaucaccucAGUCAUUCUUAUCAcuuagacaCAU |
| | | UAGUAUGUUAAGcucauccuuauCGACCAUGGCUCAGACcUGUUAcacucaaaucau |
| | | CUUAACAACUAAUGuuuaguagUCAAUACAGAUUAUGacucuaucaaaCGACCAU |
| | | GGCUCAGACcUGUUAgcaacaucaucaCAUAAUCGUAUUGAcuugcaauCUAUAUA |
| | | CUUCAAGGaagaucacuaaCGACCAUGGCUCAGACcUGUUAcacuacuaccaaCCUUG |
| | | AAUAUAUAGccguaguuCUGUUUAUUGACAUGucaacaucaauCGACCAUGGCUC |
| | | AGACcUGUUAcacuuuaccuaaCAUGUCAUAAACAGuaacagaa |
| SEQ ID NO: | Synthesized | gggUGAUAUACGACCAGUauagacuacaaCGACCAUGGCUCAGACuUGUUAgaaac |
| 301 | miR-132 | ucaagauACUGGUCUAUAUCAuuaugagcUGAUAAGAAAUGACUcaacuuaccuaCGA |
| | Imperf U | CCAUGGCUCAGACuUGUUAcaauaucaccucAGUCAUUCUUAUCAcuuagacaCAU |
| | | UAGUAUGUUAAGcuuacgcauacCGACCAUGGCUCAGACuUGUUAcacucuaauccu |
| | | CUUAACAACUAAUGuuuaguagUCAAUACAGAUUAUGacucuaucaaaCGACCAU |
| | | GGCUCAGACuUGUUAgcaacaucaucaCAUAAUCGUAUUGAcuugcaauCUAUAUA |
| | | CUUCAAGGauccucacacuCGACCAUGGCUCAGACuUGUUAcacuacauccauCCUUG |
| | | AAUAUAUAGccguaguuCUGUUUAUUGACAUGucaacaucaauCGACCAUGGCUC |
| | | AGACuUGUUAcacuuuaccuaaCAUGUCAUAAACAGuaacagaa |
| SEQ ID NO: | Synthesized | gggUGAUAUACGACCAGUgcgcacuacacAACCCCUAUCACGAUUAGCaAUUAA |
| 302 | miR-155 | gaaacucaagauACUGGUCUAUAUCAuuaugagcUGAUAAGAAAUGACUacuuuaacua |
| | Perf A | CAACCCCUAUCACGAUUAGCaAUUAAgaacacGacauCAGUCAUUCUUAUCAuu |
| | | cagacaCAUUAGUAUGUUAAGuuaagcaucauAACCCCUAUCACGAUUAGCaAUU |
| | | AAcaacgaaacgauCUUAACAACUAAUGuuuaguagUCAAUACAGAUUAUGacagcau |
| | | cuauAACCCCUAUCACGAUUAGCaAUUAAuacaccacgacgCAUAAUCGUAUUGA |
| | | uucuuucaCUAUAUACUUCAAGGaccaucacuacAACCCCUAUCACGAUUAGCaAU |
| | | UAAcacuacuagcagCCUUGAAUAUAUAGuucaugccCUGUUUAUUGACAUGucacau |
| | | cucacAACCCCUAUCACGAUUAGCaAUUAAcacaucacgcauCAUGUCAUAAACA |
| | | Guuauacaa |
| SEQ ID NO: | Synthesized | gggUGAUAUACGACCAGUucauacuacacAACCCCUAUCACGAUUAGCgAUUAA |
| 303 | miR-155 | gaaacucaauauACUGGUCUAUAUCAuuaugagcUGAUAAGAAAUGACUacaccgacaua |
| | Perf G | AACCCCUAUCACGAUUAGCgAUUAAccuacagagauuAGUCAUUCUUAUCAuuca |
| | | gacaCAUUAGUAUGUUAAGugacucaucauAACCCCUAUCACGAUUAGCgAUUA |
| | | AauucagaacgauCUUAACAACUAAUGuuuaguagUCAAUACAGAUUAUGacagcaacu |
| | | auAACCCCUAUCACGAUUAGCgAUUAAuacaccacgaauCAUAAUCGUAUUGAuu |
| | | cuuucaCUAUAUACUUCAAGGacauaccuaucAACCCCUAUCACGAUUAGCgAUU |
| | | AAcaccaugaccagCCUUGAAUAUAUAGuucaugccCUGUUUAUUGACAUGucacauc |
| | | ucacAACCCCUAUCACGAUUAGCgAUUAAcacuccacucauCAUGUCAUAAACAG |
| | | uuauacaa |
| SEQ ID NO: | Synthesized | gggUGAUAUACGACCAGUgcgcacuacacAACCCCUAUCACGAUUAGCcAUUAA |
| 304 | miR-155 | gaaacucaagauACUGGUCUAUAUCAuuaugagcUGAUAAGAAAUGACUacuuuaacua |
| | Perf C | CAACCCCUAUCACGAUUAGCcAUUAAgaacacGacauCAGUCAUUCUUAUCAuu |
| | | cagacaCAUUAGUAUGUUAAGuuaagcaucauAACCCCUAUCACGAUUAGCCAUU |
| | | AAcaacgaaacgauCUUAACAACUAAUGuuuaguagUCAAUACAGAUUAUGacagcau |
| | | cuauAACCCCUAUCACGAUUAGCcAUUAAuacaccacgacgCAUAAUCGUAUUGA |
| | | uucuuucaCUAUAUACUUCAAGGaccaucacuacAACCCCUAUCACGAUUAGCcAU |
| | | UAAcacuacuagcagCCUUGAAUAUAUAGuucaugccCUGUUUAUUGACAUGucacau |
| | | cucacAACCCCUAUCACGAUUAGCcAUUAAcacaucacgcauCAUGUCAUAAACA |
| | | Guuauacaa |
| SEQ ID NO: | Synthesized | gggUGAUAUACGACCAGUgcgcacuacacAACCCCUAUCACGAUUAGCuAUUAA |
| 305 | miR-155 | gaaacucaagauACUGGUCUAUAUCAuuaugagcUGAUAAGAAAUGACUacuuuaacua |
| | Perf U | CAACCCCUAUCACGAUUAGCuAUUAAgaacacGacauCAGUCAUUCUUAUCAuu |
| | | cagacaCAUUAGUAUGUUAAGuuaccgaucauAACCCCUAUCACGAUUAGCuAUU |
| | | AAcuuacaaacgauCUUAACAACUAAUGuuuaguagUCAAUACAGAUUAUGacaucac |
| | | gcauAACCCCUAUCACGAUUAGCuAUUAAuacaccacgacgCAUAAUCGUAUUGA |
| | | uucuuucaCUAUAUACUUCAAGGacucuaucuacAACCCCUAUCACGAUUAGCuAU |
| | | UAAcacuucacgccuCCUUGAAUAUAUAGuucaugccCUGUUUAUUGACAUGucacau |
| | | cucacAACCCCUAUCACGAUUAGCuAUUAAcacaucacgcauCAUGUCAUAAACA |
| | | Guuauacaa |
| SEQ ID NO: | Synthesized | gggUGAUAUACGACCAGUgcgcacuacacAACCCCUAUCACGUAAGCAUUAAgaa |
| 306 | miR-155 | acucaagauACUGGUCUAUAUCAuuaugagcUGAUAAGAAAUGACUauccgaacuacA |
| | Imperf ctrl | ACCCCUAUCACGUAAGCAUUAAucauccaacaugAGUCAUUCUUAUCAuucagaca |
| | | CAUUAGUAUGUUAAGugacucaucauAACCCCUAUCACGUAAGCAUUAAcuacuc |
| | | aacgauCUUAACAACUAAUGuuuaguagUCAAUACAGAUUAUGacagcaucuauAAC |
| | | CCCUAUCACGUAAGCAUUAAuacaccacgacgCAUAAUCGUAUUGAuucuuucaCU |
| | | AUAUACUUCAAGGaccaucacuacAACCCCUAUCACGUAAGCAUUAAcacuacuag |
| | | cagCCUUGAAUAUAUAGuucaugccCUGUUUAUUGACAUGucacaucucacAACCCC |
| | | UAUCACGUAAGCAUUAAcacaucacgcauCAUGUCAUAAACAGuuauacaa |
| SEQ ID NO: | Synthesized | gggUGAUAUACGACCAGUgcgcacuacacAACCCCUAUCACGUAAGCaAUUAAga |
| 307 | miR-155 | aacucaagauACUGGUCUAUAUCAuuaugagcUGAUAAGAAAUGACUauccgaacuacA |
| | Imperf A | ACCCCUAUCACGUAAGCaAUUAAucauccaacaugAGUCAUUCUUAUCAuucagac |
| | | aCAUUAGUAUGUUAAGugacucaucauAACCCCUAUCACGUAAGCaAUUAAcuac |
| | | ucaacgauCUUAACAACUAAUGuuuaguagUCAAUACAGAUUAUGacagcaucuauAA |
| | | CCCCUAUCACGUAAGCaAUUAAuacaccacgacgCAUAAUCGUAUUGAuucuuuca |
| | | CUAUAUACUUCAAGGaccaucacuacAACCCCUAUCACGUAAGCaAUUAAcacuac |
| | | uagcagCCUUGAAUAUAUAGuucaugccCUGUUUAUUGACAUGucacaucucacAAC |
| | | CCCUAUCACGUAAGCaAUUAAcacaucacgcauCAUGUCAUAAACAGuuauacaa |
| SEQ ID NO: | Synthesized | gggUGAUAUACGACCAGUucagacuacacAACCCCUAUCACGUAAGCgAUUAAga |
| 308 | miR-155 | aacucaauauACUGGUCUAUAUCAuuaugagcUGAUAAGAAAUGACUacaccgacauaA |
| | Imperf G | ACCCCUAUCACGUAAGCgAUUAAccuacagagauuAGUCAUUCUUAUCAuucagac |
| | | aCAUUAGUAUGUUAAGugacucagcauAACCCCUAUCACGUAAGCgAUUAAauuc |
| | | agaacaauCUUAACAACUAAUGuuuaguagUCAAUACAGAUUAUGacagcaacuauAA |
| | | CCCCUAUCACGUAAGCgAUUAAuacaccacgaauCAUAAUCGUAUUGAuucuuuca |
| | | CUAUAUACUUCAAGGacauaccuaucAACCCCUAUCACGUAAGCgAUUAAcacca |
| | | ucaccagCCUUGAAUAUAUAGuucaugccCUGUUUAUUGACAUGucacaucucacAAC |
| | | CCCUAUCACGUAAGCgAUUAAcacuccacucauCAUGUCAUAAACAGuuauacaa |
| SEQ ID NO: | Synthesized | gggUGAUAUACGACCAGUgcgcacuacacAACCCCUAUCACGUAAGCcAUUAAga |
| 309 | miR-155 | aacucaagauACUGGUCUAUAUCAuuaugagcUGAUAAGAAAUGACUauccgaacuacA |
| | Imperf C | ACCCCUAUCACGUAAGCcAUUAAucauccaacaugAGUCAUUCUUAUCAuucagac |
| | | aCAUUAGUAUGUUAAGugacucaucauAACCCCUAUCACGUAAGCcAUUAAcuac |
| | | ucaacgauCUUAACAACUAAUGuuuaguagUCAAUACAGAUUAUGacagcaucuauAA |
| | | CCCCUAUCACGUAAGCcAUUAAuacaccacgacgCAUAAUCGUAUUGAuucuuuca |
| | | CUAUAUACUUCAAGGaccaucacuacAACCCCUAUCACGUAAGCcAUUAAcacuac |
| | | uagcagCCUUGAAUAUAUAGuucaugccCUGUUUAUUGACAUGucacaucucacAAC |
| | | CCCUAUCACGUAAGCcAUUAAcacaucacgcauCAUGUCAUAAACAGuuauacaa |
| SEQ ID NO: | Synthesized | gggUGAUAUACGACCAGUgcgcacuacacAACCCCUAUCACGUAAGCuAUUAAga |
| 310 | miR-155 | aacucaagauACUGGUCUAUAUCAuuaugagcUGAUAAGAAAUGACUauccgaacuacA |
| | Imperf U | ACCCCUAUCACGUAAGCuAUUAAucauccaacaugAGUCAUUCUUAUCAuucagac |
| | | aCAUUAGUAUGUUAAGugacucaucauAACCCCUAUCACGUAAGCuAUUAAcuac |
| | | ucaacgauCUUAACAACUAAUGuuuaguagUCAAUACAGAUUAUGacaccaucuauAA |
| | | CCCCUAUCACGUAAGCuAUUAAuacaccacgacgCAUAAUCGUAUUGAuucuuuca |
| | | CUAUAUACUUCAAGGaccaucacaacAACCCCUAUCACGUAAGCuAUUAAcacgac |
| | | cacgagCCUUGAAUAUAUAGuucaugccCUGUUUAUUGACAUGucacaucucacAAC |
| | | CCCUAUCACGUAAGCuAUUAAcacaucacgcauCAUGUCAUAAACAGuuauacaa |
| SEQ ID NO: | Synthesized | gggUGAUAUACGACCAGUccgcacuacacCUAUCUGCACUAGAUGCACCUUAuaa |
| 311 | miR-18a | acucagaauACUGGUCUAUAUCAuuaugagcUGAUAAGAAAUGACUagcucaaguccC |
| | Perf ctrl | UAUCUGCACUAGAUGCACCUUAauaaguccuccgAGUCAUUCUUAUCAuucagaca |
| | | CAUUAGUAUGUUAAGucacucacgauCUAUCUGCACUAGAUGCACCUUAcuacuc |
| | | aacuauCUUAACAACUAAUGuuuaguagUCAAUACAGAUUAUGacagcauagauCUA |
| | | UCUGCACUAGAUGCACCUUAuacaccacgacgCAUAAUCGUAUUGAuucuuucaCU |
| | | AUAUACUUCAAGGaccaucacuacCUAUCUGCACUAGAUGCACCUUAcacuacuacc |
| | | agCCUUGAAUAUAUAGuucaugccCUGUUUAUUGACAUGucacaucucacCUAUCU |
| | | GCACUAGAUGCACCUUAcacaucacgcauCAUGUCAUAAACAGuuauacaa |
| SEQ ID NO: | Synthesized | gggUGAUAUACGACCAGUccgcacuacacCUAUCUGCACUAGAUGCAaCCUUAua |
| 312 | miR-18a | aacucagaauACUGGUCUAUAUCAuuaugagcUGAUAAGAAAUGACUagcucaaguccC |
| | Perf A | UAUCUGCACUAGAUGCAaCCUUAauaaguccuccgAGUCAUUCUUAUCAuucagac |
| | | aCAUUAGUAUGUUAAGucacucacgauCUAUCUGCACUAGAUGCAaCCUUAcuac |
| | | ucaacuauCUUAACAACUAAUGuuuaguagUCAAUACAGAUUAUGacagcauagauCU |
| | | AUCUGCACUAGAUGCAaCCUUAuacaccacgacgCAUAAUCGUAUUGAuucuuuca |
| | | CUAUAUACUUCAAGGaccaucacuacCUAUCUGCACUAGAUGCAaCCUUAcacuac |
| | | uaccagCCUUGAAUAUAUAGuucaugccCUGUUUAUUGACAUGucacaucucacCUA |
| | | UCUGCACUAGAUGCAaCCUUAcacaucacgcauCAUGUCAUAAACAGuuauacaa |
| SEQ ID NO: | Synthesized | gggUGAUAUACGACCAGUccgcacuacacCUAUCUGCACUAGAUGCAgCCUUAua |
| 313 | miR-18a | aacucagaauACUGGUCUAUAUCAuuaugagcUGAUAAGAAAUGACUagcucaaguccC |
| | Perf G | UAUCUGCACUAGAUGCAgCCUUAauaaguccuccgAGUCAUUCUUAUCAuucagac |
| | | aCAUUAGUAUGUUAAGucacucacgauCUAUCUGCACUAGAUGCAgCCUUAcuac |
| | | ucaacuauCUUAACAACUAAUGuuuaguagUCAAUACAGAUUAUGacagcauagauCU |
| | | AUCUGCACUAGAUGCAgCCUUAuacaccacgacgCAUAAUCGUAUUGAuucuuuca |
| | | CUAUAUACUUCAAGGaccaucacuacCUAUCUGCACUAGAUGCAgCCUUAcacua |
| | | cuaccagCCUUGAAUAUAUAGuucaugccCUGUUUAUUGACAUGucacaucucacCUA |
| | | UCUGCACUAGAUGCAgCCUUAcacaucacgcauCAUGUCAUAAACAGuuauacaa |
| SEQ ID NO: | Synthesized | gggUGAUAUACGACCAGUccgcacuacacCUAUCUGCACUAGAUGCAcCCUUAua |
| 314 | miR-18a | aacucagaauACUGGUCUAUAUCAuuaugagcUGAUAAGAAAUGACUagcucaaguccC |
| | Perf C | UAUCUGCACUAGAUGCAcCCUUAauaaguccuccgAGUCAUUCUUAUCAuucagac |
| | | aCAUUAGUAUGUUAAGucacucacgauCUAUCUGCACUAGAUGCAcCCUUAcuac |
| | | ucaacuauCUUAACAACUAAUGuuuaguagUCAAUACAGAUUAUGacagcauagauCU |
| | | AUCUGCACUAGAUGCAcCCUUAuacaccacgacgCAUAAUCGUAUUGAuucuuuca |
| | | CUAUAUACUUCAAGGaccaucacuacCUAUCUGCACUAGAUGCAcCCUUAcacuac |
| | | uaccagCCUUGAAUAUAUAGuucaugccCUGUUUAUUGACAUGucacaucucacCUA |
| | | UCUGCACUAGAUGCAcCCUUAcacaucacgcauCAUGUCAUAAACAGuuauacaa |
| SEQ ID NO: | Synthesized | gggUGAUAUACGACCAGUccgcacuacacCUAUCUGCACUAGAUGCAuCCUUAua |
| 315 | miR-18a | aacucagaauACUGGUCUAUAUCAuuaugagcUGAUAAGAAAUGACUagcucaaguccC |
| | Perf U | UAUCUGCACUAGAUGCAuCCUUAauaaguccuccgAGUCAUUCUUAUCAuucagac |
| | | aCAUUAGUAUGUUAAGucacucacuauCUAUCUGCACUAGAUGCAuCCUUAcuac |
| | | ucaacuauCUUAACAACUAAUGuuuaguagUCAAUACAGAUUAUGacagcauagacCU |
| | | AUCUGCACUAGAUGCAuCCUUAuacaccacgacgCAUAAUCGUAUUGAuucuuuca |
| | | CUAUAUACUUCAAGGaccaucacuacCUAUCUGCACUAGAUGCAuCCUUAcacua |
| | | cuaccagCCUUGAAUAUAUAGuucaugccCUGUUUAUUGACAUGucacaucucacCUA |
| | | UCUGCACUAGAUGCAuCCUUAcacaucacgcauCAUGUCAUAAACAGuuauacaa |
| SEQ ID NO: | Synthesized | gggUGAUAUACGACCAGUccgcacuacacCUAUCUGCACUACUGCACCUUAuaaac |
| 316 | miR-18a | ucagaauACUGGUCUAUAUCAuuaugagcUGAUAAGAAAUGACUucuucaaguccCU |
| | Imperf ctrl | AUCUGCACUACUGCACCUUAauaaguccuccuAGUCAUUCUUAUCAuucagacaCA |
| | | UUAGUAUGUUAAGugacucaucauCUAUCUGCACUACUGCACCUUAcuacucaacga |
| | | uCUUAACAACUAAUGuuuaguagUCAAUACAGAUUAUGacagcaucuauCUAUCU |
| | | GCACUACUGCACCUUAuacaccacgacgCAUAAUCGUAUUGAuucuuucaCUAUAU |
| | | ACUUCAAGGaccaucacuacCUAUCUGCACUACUGCACCUUAcacuacuagcagCCU |
| | | UGAAUAUAUAGuucaugccCUGUUUAUUGACAUGucacaucucacCUAUCUGCAC |
| | | UACUGCACCUUAcacaucacgcauCAUGUCAUAAACAGuuauacaa |
| SEQ ID NO: | Synthesized | gggUGAUAUACGACCAGUccgcacuacacCUAUCUGCACUACUGCAaCCUUAuaaa |
| 317 | miR-18a | cucagaauACUGGUCUAUAUCAuuaugagcUGAUAAGAAAUGACUucuucaaguccCU |
| | Imperf A | AUCUGCACUACUGCAaCCUUAauaaguccuccuAGUCAUUCUUAUCAuucagacaC |
| | | AUUAGUAUGUUAAGucacucaucauCUAUCUGCACUACUGCAaCCUUAcuacucaa |
| | | cuguCUUAACAACUAAUGuuuaguagUCAAUACAGAUUAUGacagcauagauCUAU |
| | | CUGCACUACUGCAaCCUUAuacaccacgacgCAUAAUCGUAUUGAuucuuucaCUA |
| | | UAUACUUCAAGGaccaucacuacCUAUCUGCACUACUGCAaCCUUAcacuacuaccag |
| | | CCUUGAAUAUAUAGuucaugccCUGUUUAUUGACAUGucacaucucacCUAUCUG |
| | | CACUACUGCAaCCUUAcacaucacgcauCAUGUCAUAAACAGuuauacaa |
| SEQ ID NO: | Synthesized | gggUGAUAUACGACCAGUccgcacuacacCUAUCUGCACUACUGCAgCCUUAuaaa |
| 318 | miR-18a | cucagaauACUGGUCUAUAUCAuuaugagcUGAUAAGAAAUGACUucuucaaguccCU |
| | Imperf G | AUCUGCACUACUGCAgCCUUAauaaguccuccuAGUCAUUCUUAUCAuucagacaC |
| | | AUUAGUAUGUUAAGucacucaucauCUAUCUGCACUACUGCAgCCUUAcuacucaa |
| | | cuguCUUAACAACUAAUGuuuaguagUCAAUACAGAUUAUGacagcauagauCUAU |
| | | CUGCACUACUGCAgCCUUAuacaccacgacgCAUAAUCGUAUUGAuucuuucaCUA |
| | | UAUACUUCAAGGaccaucacuacCUAUCUGCACUACUGCAgCCUUAcacuacuaccag |
| | | CCUUGAAUAUAUAGuucaugccCUGUUUAUUGACAUGucacaucucacCUAUCUG |
| | | CACUACUGCAgCCUUAcacaucacgcauCAUGUCAUAAACAGuuauacaa |
| SEQ ID NO: | Synthesized | gggUGAUAUACGACCAGUccgcacuacacCUAUCUGCACUACUGCAcCCUUAuaaa |
| 319 | miR-18a | cucagaauACUGGUCUAUAUCAuuaugagcUGAUAAGAAAUGACUucuucaaguccCU |
| | Imperf C | AUCUGCACUACUGCAcCCUUAauaaguccuccuAGUCAUUCUUAUCAuucagacaC |
| | | AUUAGUAUGUUAAGucacucaucauCUAUCUGCACUACUGCAcCCUUAcuacucaa |
| | | cuguCUUAACAACUAAUGuuuaguagUCAAUACAGAUUAUGacagcauagauCUAU |
| | | CUGCACUACUGCAcCCUUAuacaccacgacgCAUAAUCGUAUUGAuucuuucaCUA |
| | | UAUACUUCAAGGaccaucacuacCUAUCUGCACUACUGCAcCCUUAcacuacuaccag |
| | | CCUUGAAUAUAUAGuucaugccCUGUUUAUUGACAUGucacaucucacCUAUCUG |
| | | CACUACUGCAcCCUUAcacaucacgcauCAUGUCAUAAACAGuuauacaa |
| SEQ ID NO: | Synthesized | gggUGAUAUACGACCAGUccgcacuacacCUAUCUGCACUACUGCAuCCUUAuaaa |
| 320 | miR-18a | cucagaauACUGGUCUAUAUCAuuaugagcUGAUAAGAAAUGACUucuucaaguccCU |
| | Imperf U | AUCUGCACUACUGCAuCCUUAauaaguccuccuAGUCAUUCUUAUCAuucagacaC |
| | | AUUAGUAUGUUAAGucacucaucauCUAUCUGCACUACUGCAuCCUUAcuacucaa |
| | | cuguCUUAACAACUAAUGuuuaguagUCAAUACAGAUUAUGacagcauagauCUAU |
| | | CUGCACUACUGCAuCCUUAuacaccacgacgCAUAAUCGUAUUGAuucuuucaCUA |
| | | UAUACUUCAAGGaccaucacuacCUAUCUGCACUACUGCAuCCUUAcacuacuaccag |
| | | CCUUGAAUAUAUAGuucaugccCUGUUUAUUGACAUGucacaucucacCUAUCUG |
| | | CACUACUGCAuCCUUAcacaucacgcauCAUGUCAUAAACAGuuauacaa |
| SEQ ID NO: | Synthesized | gggUGAUAUACGACCAGUccucacuacuaCUACCUGCACUGUAAGCACUUUGacu |
| 321 | miR-17 | ucgccgauuACUGGUCUAUAUCAuuaugagcUGAUAAGAAAUGACUauccugacuacC |
| | Perf ctrl | UACCUGCACUGUAAGCACUUUGacauccuaccugAGUCAUUCUUAUCAuucagaca |
| | | CAUUAGUAUGUUAAGucaagccacauCUACCUGCACUGUAAGCACUUUGcuaagc |
| | | gacacuCUUAACAACUAAUGuuuaguagUCAAUACAGAUUAUGacuuccgaucuCUA |
| | | CCUGCACUGUAAGCACUUUGuccuauuacgucCAUAAUCGUAUUGAuucuuucaCU |
| | | AUAUACUUCAAGGaccaucacgaaCUACCUGCACUGUAAGCACUUUGaccuacuacc |
| | | auCCUUGAAUAUAUAGuucaugccCUGUUUAUUGACAUGucacuccauacCUACCU |
| | | GCACUGUAAGCACUUUGaccauuccucuuCAUGUCAUAAACAGuuauacaa |
| SEQ ID NO: | Synthesized | gggUGAUAUACGACCAGUccucacuacuaCUACCUGCACUGUAAGCAaCUUUGac |
| 322 | miR-17 | uucgccgauuACUGGUCUAUAUCAuuaugagcUGAUAAGAAAUGACUauccugacuac |
| | Perf A | CUACCUGCACUGUAAGCAaCUUUGacauccuaccugAGUCAUUCUUAUCAuucaga |
| | | caCAUUAGUAUGUUAAGucaagccacauCUACCUGCACUGUAAGCAaCUUUGcua |
| | | agcgacacuCUUAACAACUAAUGuuuaguagUCAAUACAGAUUAUGacuuccgaucuC |
| | | UACCUGCACUGUAAGCAaCUUUGuccuauuacgucCAUAAUCGUAUUGAuucuuuc |
| | | aCUAUAUACUUCAAGGaccaucacgaaCUACCUGCACUGUAAGCAaCUUUGaccua |
| | | cuaccauCCUUGAAUAUAUAGuucaugccCUGUUUAUUGACAUGucacuccauacCUA |
| | | CCUGCACUGUAAGCAaCUUUGaccauuccucuuCAUGUCAUAAACAGuuauacaa |
| SEQ ID NO: | Synthesized | gggUGAUAUACGACCAGUccucacuacuaCUACCUGCACUGUAAGCAgCUUUGac |
| 323 | miR-17 | uucgccgauuACUGGUCUAUAUCAuuaugagcUGAUAAGAAAUGACUauccugacuac |
| | Perf G | CUACCUGCACUGUAAGCAgCUUUGacauccuaccugAGUCAUUCUUAUCAuucaga |
| | | caCAUUAGUAUGUUAAGucaagccacauCUACCUGCACUGUAAGCAgCUUUGcua |
| | | agcgacacuCUUAACAACUAAUGuuuaguagUCAAUACAGAUUAUGacuuccgaucuC |
| | | UACCUGCACUGUAAGCAgCUUUGuccuauuacgucCAUAAUCGUAUUGAuucuuu |
| | | caCUAUAUACUUCAAGGaccaucacgaaCUACCUGCACUGUAAGCAgCUUUGacc |
| | | uacuaccauCCUUGAAUAUAUAGuucaugccCUGUUUAUUGACAUGucacuccauacC |
| | | UACCUGCACUGUAAGCAgCUUUGaccauuccucuuCAUGUCAUAAACAGuuauaca |
| | | a |
| SEQ ID NO: | Synthesized | gggUGAUAUACGACCAGUccucacuacuaCUACCUGCACUGUAAGCAcCUUUGac |
| 324 | miR-17 | uucgccgauuACUGGUCUAUAUCAuuaugagcUGAUAAGAAAUGACUauccugacuac |
| | Perf C | CUACCUGCACUGUAAGCAcCUUUGacauccuaccugAGUCAUUCUUAUCAuucaga |
| | | caCAUUAGUAUGUUAAGucaagccacauCUACCUGCACUGUAAGCAcCUUUGcua |
| | | agcgacacuCUUAACAACUAAUGuuuaguagUCAAUACAGAUUAUGacuuccgaucuC |
| | | UACCUGCACUGUAAGCAcCUUUGuccuauuacgucCAUAAUCGUAUUGAuucuuuc |
| | | aCUAUAUACUUCAAGGaccaucacgaaCUACCUGCACUGUAAGCAcCUUUGaccua |
| | | cuaccauCCUUGAAUAUAUAGuucaugccCUGUUUAUUGACAUGucacuccauacCUA |
| | | CCUGCACUGUAAGCAcCUUUGaccauuccucuuCAUGUCAUAAACAGuuauacaa |
| SEQ ID NO: | Synthesized | gggUGAUAUACGACCAGUccucacuacuaCUACCUGCACUGUAAGCAuCUUUGac |
| 325 | miR-17 | uucgccgauuACUGGUCUAUAUCAuuaugagcUGAUAAGAAAUGACUauccugacuac |
| | Perf U | CUACCUGCACUGUAAGCAuCUUUGacauccuaccugAGUCAUUCUUAUCAuucaga |
| | | caCAUUAGUAUGUUAAGucaugacacacCUACCUGCACUGUAAGCAuCUUUGaca |
| | | uccgacacuCUUAACAACUAAUGuuuaguagUCAAUACAGAUUAUGacuuccgaucuC |
| | | caCUAUAUACUUCAAGGaccaucacgaaCUACCUGCACUGUAAGCAuCUUUGacc |
| | | uacuaccauCCUUGAAUAUAUAGuucaugccCUGUUUAUUGACAUGucacuccauacC |
| | | UACCUGCACUGUAAGCAuCUUUGaccauuccucuuCAUGUCAUAAACAGuuauaca |
| | | a |
| SEQ ID NO: | Synthesized | gggUGAUAUACGACCAGUccgcacuacacCUACCUGCACUGAUGCACUUUGuaaac |
| 326 | miR-17 | uccugauACUGGUCUAUAUCAuuaugagcUGAUAAGAAAUGACUauccgaacuacCUA |
| | Imperf ctrl | CCUGCACUGAUGCACUUUGccauccaaccugAGUCAUUCUUAUCAuucagacaCAU |
| | | UAGUAUGUUAAGugacuccucauCUACCUGCACUGAUGCACUUUGcuacucaacuau |
| | | CUUAACAACUAAUGuuuaguagUCAAUACAGAUUAUGacaucauauauCUACCUG |
| | | CACUGAUGCACUUUGcccaccucgacgCAUAAUCGUAUUGAuucuuucaCUAUAUA |
| | | CUUCAAGGaccaucacuauCUACCUGCACUGAUGCACUUUGuccuacuaccagCCUU |
| | | GAAUAUAUAGuucaugccCUGUUUAUUGACAUGucacaucucacCUACCUGCACU |
| | | GAUGCACUUUGcauaacacucauCAUGUCAUAAACAGuuauacaa |
| SEQ ID NO: | Synthesized | gggUGAUAUACGACCAGUccauacgacacCUACCUGCACUGAUGCAaCUUUGcuaa |
| 327 | miR-17 | cugaucauACUGGUCUAUAUCAuuaugagcUGAUAAGAAAUGACUaucegaacuacCU |
| | Imperf A | ACCUGCACUGAUGCAaCUUUGacaugcugacugAGUCAUUCUUAUCAuucagacaC |
| | | AUUAGUAUGUUAAGugacuccacauCUACCUGCACUGAUGCAaCUUUGcuacgccu |
| | | cuauCUUAACAACUAAUGuuuaguagUCAAUACAGAUUAUGacuuucacgccCUACC |
| | | UGCACUGAUGCAaCUUUGacuacuuuaccgCAUAAUCGUAUUGAuucuuucaCUAU |
| | | AUACUUCAAGGagucucacgcuCUACCUGCACUGAUGCAaCUUUGuacuacuaccag |
| | | CCUUGAAUAUAUAGuucaugccCUGUUUAUUGACAUGcgacaccacucCUACCUGC |
| | | ACUGAUGCAaCUUUGcugaacacugauCAUGUCAUAAACAGuuauacaa |
| SEQ ID NO: | Synthesized | gggUGAUAUACGACCAGUccauacgacacCUACCUGCACUGAUGCAgCUUUGcuaa |
| 328 | miR-17 | cugaucauACUGGUCUAUAUCAuuaugagcUGAUAAGAAAUGACUauccgaacuacCU |
| | Imperf G | ACCUGCACUGAUGCAgCUUUGacaugcugacugAGUCAUUCUUAUCAuucagacaC |
| | | AUUAGUAUGUUAAGugacuccacauCUACCUGCACUGAUGCAgCUUUGcuacgccu |
| | | cuauCUUAACAACUAAUGuuuaguagUCAAUACAGAUUAUGacuuucacgccCUACC |
| | | UGCACUGAUGCAgCUUUGacuacuuuaccgCAUAAUCGUAUUGAuucuuucaCUAU |
| | | AUACUUCAAGGagucucacgcuCUACCUGCACUGAUGCAgCUUUGuacuacuaccag |
| | | CCUUGAAUAUAUAGuucaugccCUGUUUAUUGACAUGegacaccacucCUACCUGC |
| | | ACUGAUGCAgCUUUGcugaacacugauCAUGUCAUAAACAGuuauacaa |
| SEQ ID NO: | Synthesized | gggUGAUAUACGACCAGUccauacgacacCUACCUGCACUGAUGCAcCUUUGcuaa |
| 329 | miR-17 | cugaucauACUGGUCUAUAUCAuuaugagcUGAUAAGAAAUGACUauccgaacuacCU |
| | Imperf C | ACCUGCACUGAUGCAcCUUUGacaugcugacugAGUCAUUCUUAUCAuucagacaC |
| | | AUUAGUAUGUUAAGugacuccacauCUACCUGCACUGAUGCAcCUUUGcuacgccu |
| | | cuauCUUAACAACUAAUGuuuaguagUCAAUACAGAUUAUGacuuucacgccCUACC |
| | | UGCACUGAUGCAcCUUUGacuacuuuaccgCAUAAUCGUAUUGAuucuuucaCUAU |
| | | AUACUUCAAGGagucucacgcuCUACCUGCACUGAUGCAcCUUUGuacuacuaccag |
| | | CCUUGAAUAUAUAGuucaugccCUGUUUAUUGACAUGcgacaccacucCUACCUGC |
| | | ACUGAUGCAcCUUUGcugaacacugauCAUGUCAUAAACAGuuauacaa |
| SEQ ID NO: | Synthesized | gggUGAUAUACGACCAGUccucacuacuaCUACCUGCACUGAUGCAuCUUUGacu |
| 330 | miR-17 | ucgccgauuACUGGUCUAUAUCAuuaugagcUGAUAAGAAAUGACUauccugacuacC |
| | Imperf U | UACCUGCACUGAUGCAuCUUUGaccugcuaccugAGUCAUUCUUAUCAuucagaca |
| | | CAUUAGUAUGUUAAGucaagccacauCUACCUGCACUGAUGCAuCUUUGcuaagc |
| | | gacacuCUUAACAACUAAUGuuuaguagUCAAUACAGAUUAUGacuuccgaucuCUA |
| | | CCUGCACUGAUGCAuCUUUGuccuauuacgucCAUAAUCGUAUUGAuucuuucaCU |
| | | AUAUACUUCAAGGaccuucacgaaCUACCUGCACUGAUGCAuCUUUGaccuacuacc |
| | | auCCUUGAAUAUAUAGuucaugccCUGUUUAUUGACAUGucacuccauacCUACCU |
| | | GCACUGAUGCAuCUUUGaccauuccucuuCAUGUCAUAAACAGuuauacaa |

### Example 5: Determining the Number of Binding Sites in an Engineered sfRNA Targeting miR-155

sfRNAs were tested with varying numbers of imperf miR-155 binding sites. The engineered RNAs were tested for their ability to bind miR-155 mimics and rescue luciferase activity. Briefly, the functional efficacy of the number of binding sites in an engineered polynucleotide was validated in a luciferase rescue assay. miRNA binding sites (MBS) for miR-155 was inserted into the 3′-UTR of a Renilla luciferase control construct from a dual-luciferase reporter system.

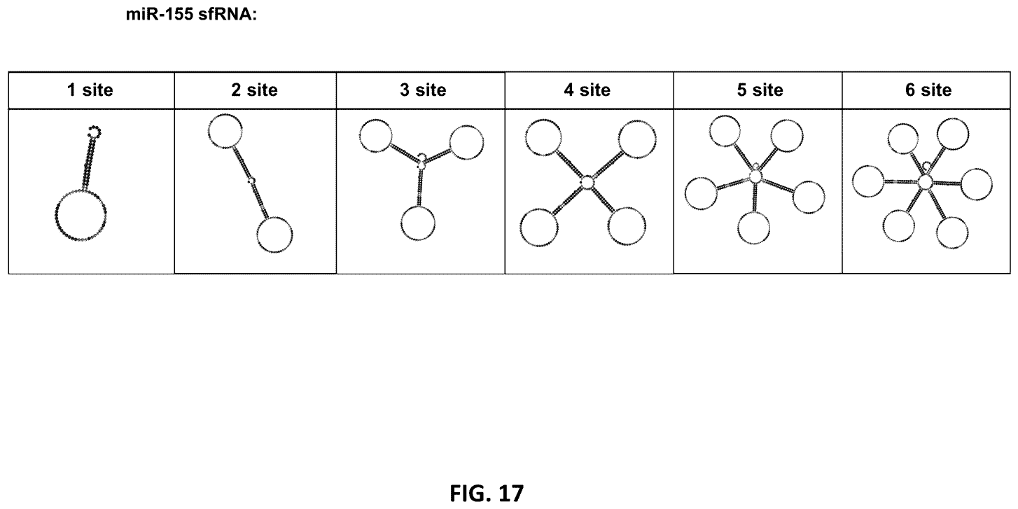

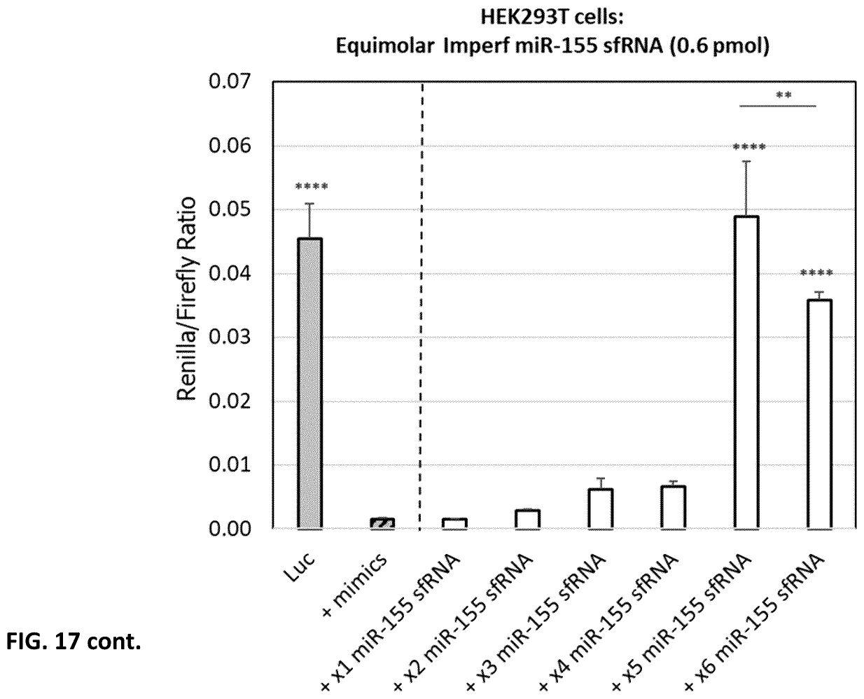

HEK293T cells were co-transfected with reporter plasmids, miR-155 mimics and/or respective engineered polynucleotides (snowflake RNA-sfRNA) targeting the miR-155 mimics for 48 h at 0.6 pmol (equimolar) concentrations to determine the effect of the number of binding sites on the sfRNA. The sfRNA was tested with 1, 2, 3, 4, 5, and 6 binding sites as shown in FIG. 17. Also FIG. 17, shows that 5 or 6 binding sites on a sfRNA were the most effective in sponging and reducing the effect of miR-155 on the luciferase expression. Notably, 5 binding sites showed a greater luciferase rescue effect compared to 6 binding sites.

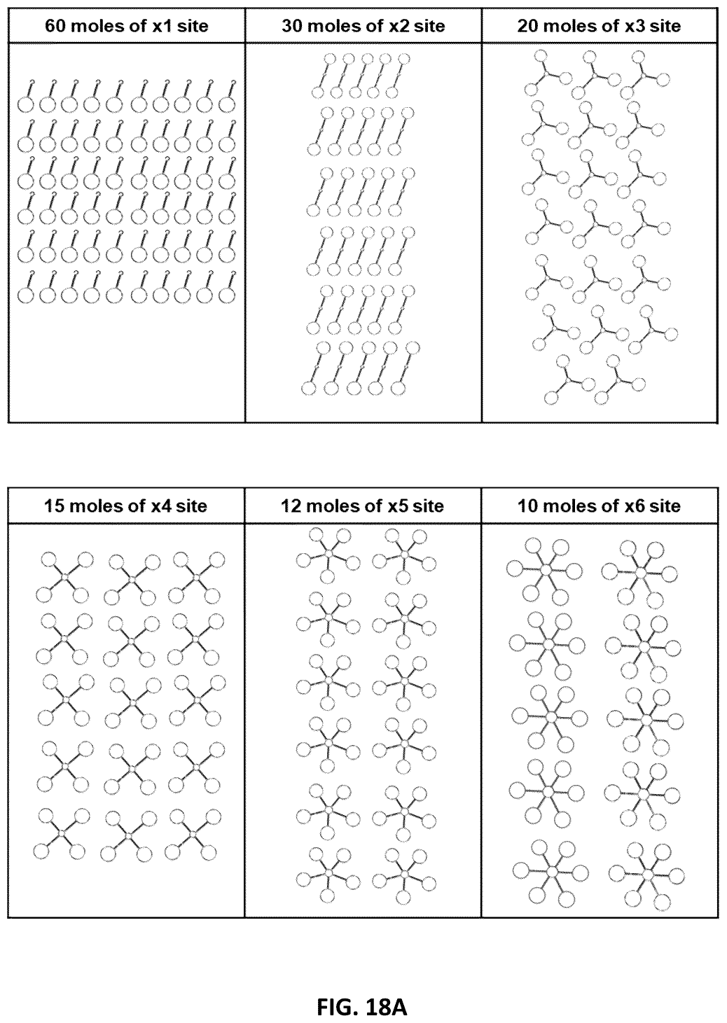

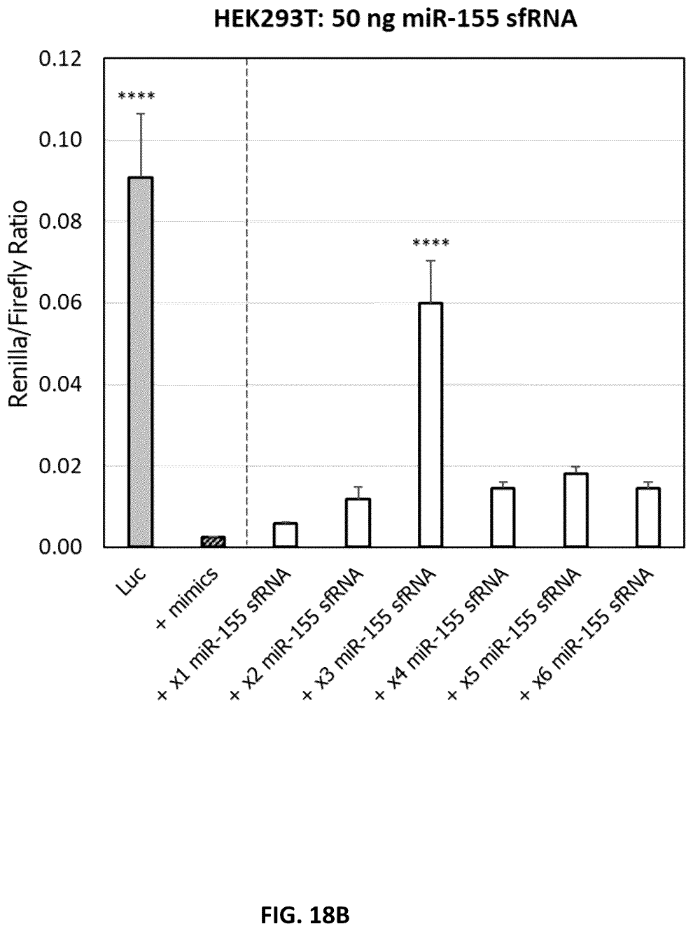

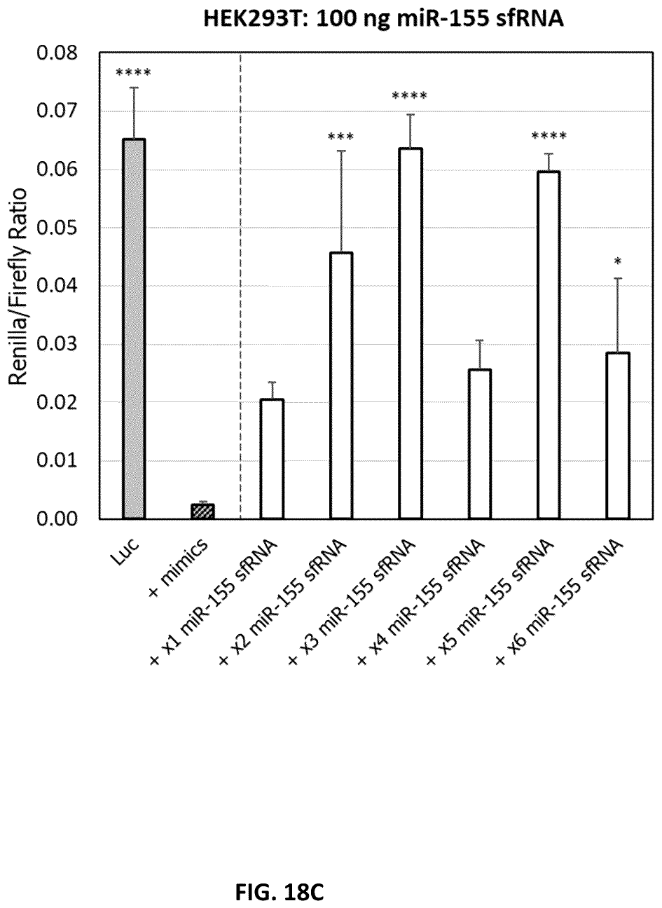

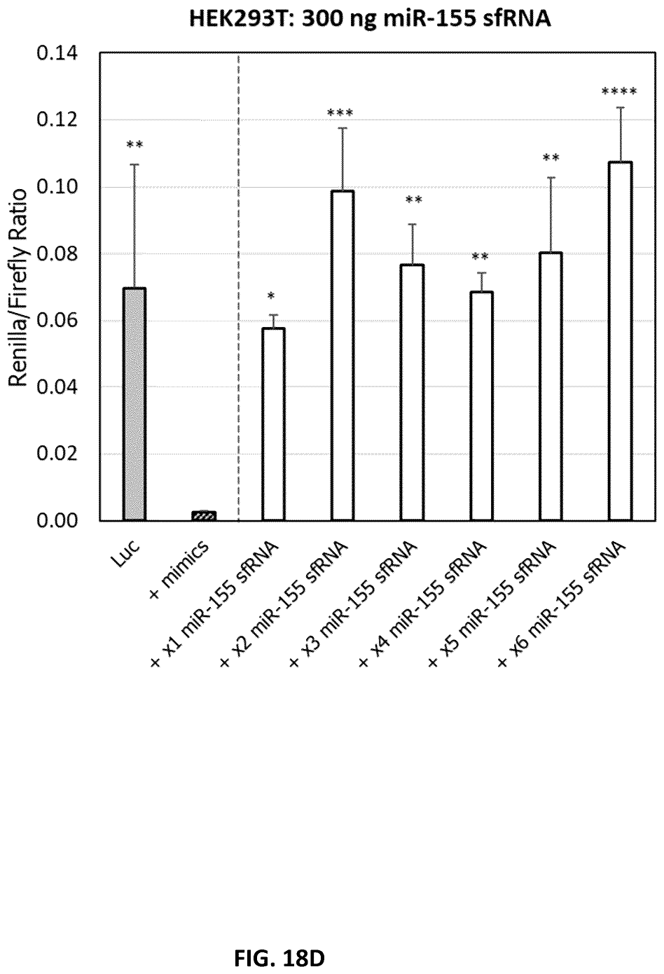

Additionally, an equal number of binding sites presented by sfRNAs with varying numbers of binding sites were tested in their ability to sponge miR-155 in the dual-luciferase reporter system. The following molar ratios of sfRNA were transfected at either 50, 100 or 300 ng to achieve a total number of 60 binding sites: 60 moles of 1 binding site, 30 moles of 2 binding sites, 20 moles of 3 binding sites, 15 moles of 4 binding sites, 12 moles of 5 binding sites, and 10 moles of 6 binding sites as shown in FIGS. 18A-D. FIG. 18B and FIG. 18C show that at 50 and 100 ng of sfRNA, 20 moles of the 3 binding site sfRNA was most effective at sponging the nucleic acid. At 300 ng, the effect was saturated and there was no significant difference between sfRNAs of different binding sites (FIG. 18D).

### Example 6: Determining Linker Size in sfRNA Targeting miR-155

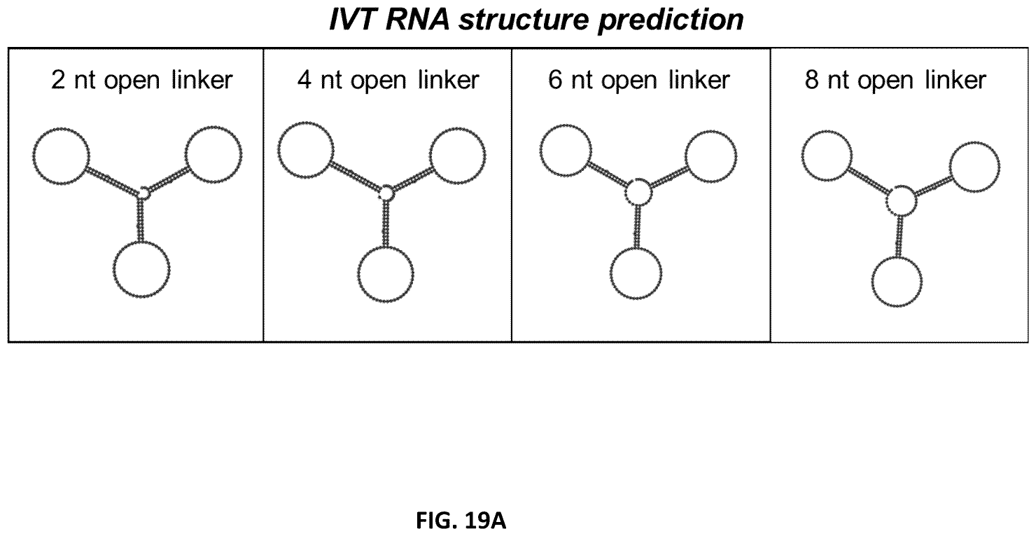

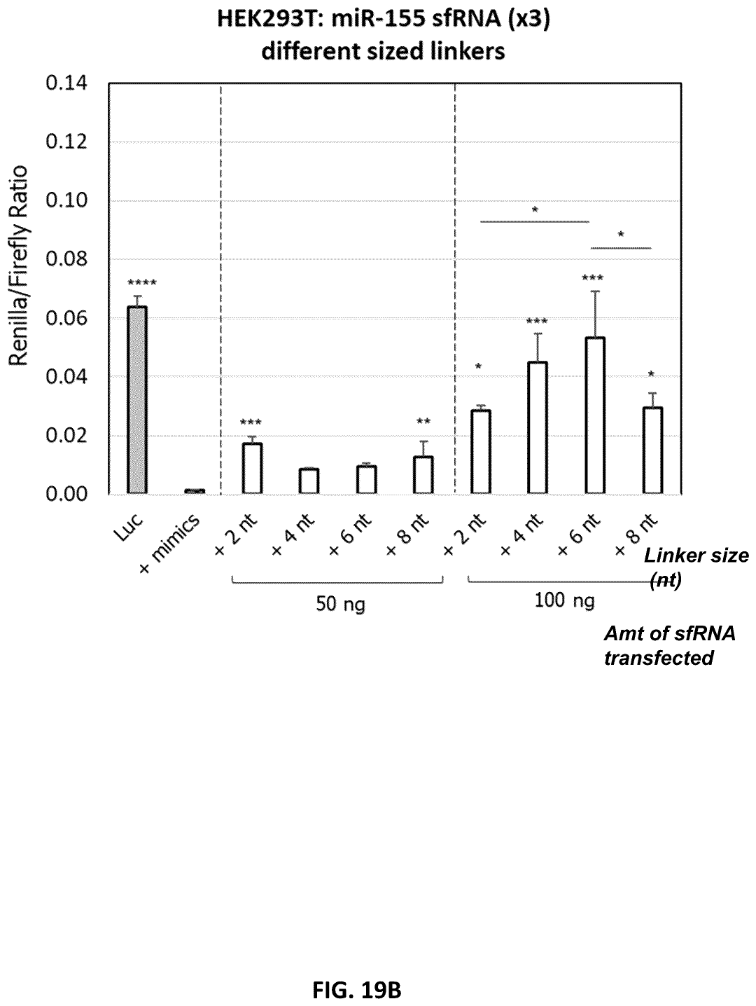

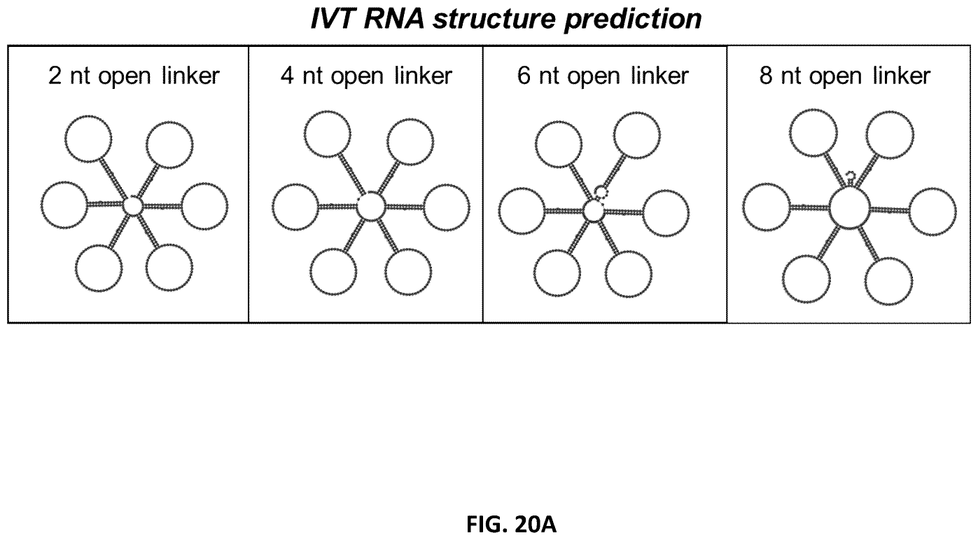

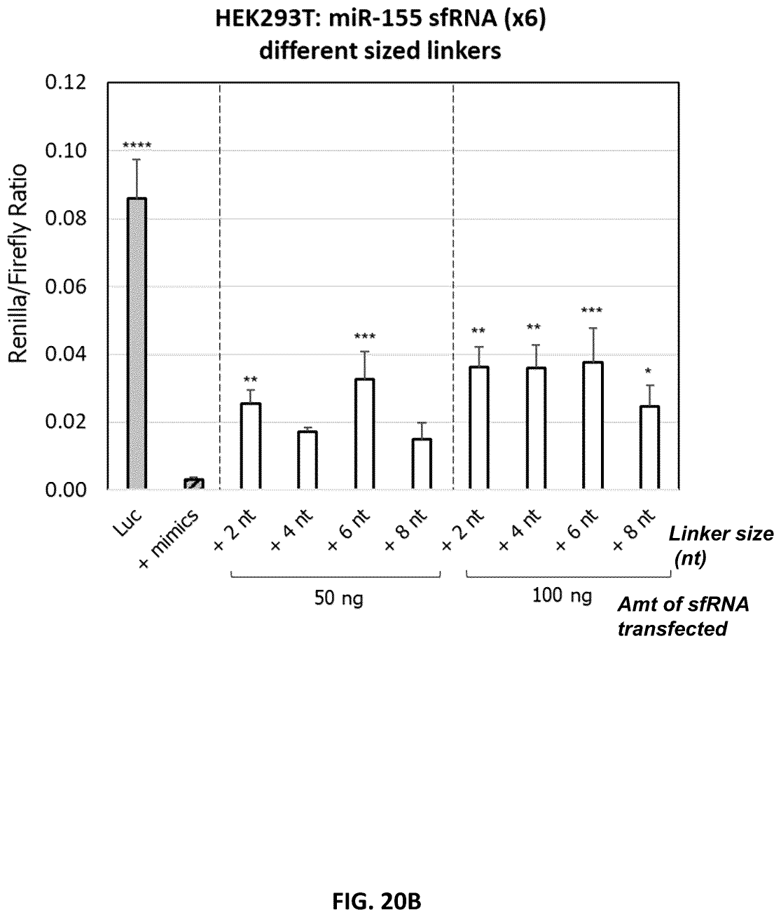

Engineered RNAs were tested with varying linker sizes. 2 nucleotide (nt), 4 nt, 6 nt, and 8 nt linker sizes between stem loops were tested in sfRNAs with 3 and 6 binding sites as shown in FIG. 19A and FIG. 20A. The engineered RNAs were tested for their ability to bind miR-155 mimics. Briefly, the functional efficacy of the linker sizes in an engineered polynucleotide was validated in a dual-luciferase reporter system assay described above.

HEK293T cells were transfected with 25 ng luciferase vector, 0.5 pmol of miR-155 mimics and various doses (50 100 ng) of in vitro T7 synthesized sfRNAs with different sized linkers (2, 4, 6, 8 nt) using Lipofectamine 2000 reagent. The luciferase assay was completed 48 hours post transfection. FIG. 19B and FIG. 20B shows luciferase results for sfRNA with different sized linkers for 3 and 6 binding sites respectively. FIG. 19B shows that at a 100 ng dose, 3 site sfRNA with 6 nt linkers were more effective than 2 nt and 8 nt linkers. Additionally, at a 100 ng dose, 6 site sfRNA showed similar effects across all linker sizes (FIG. 20B).

### Example 7: Determining Stem Design in sfRNA Targeting miR-155

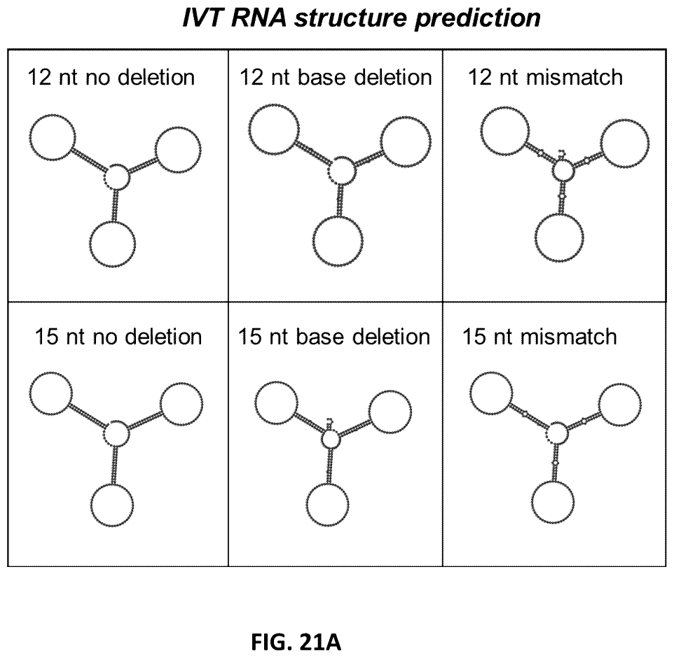

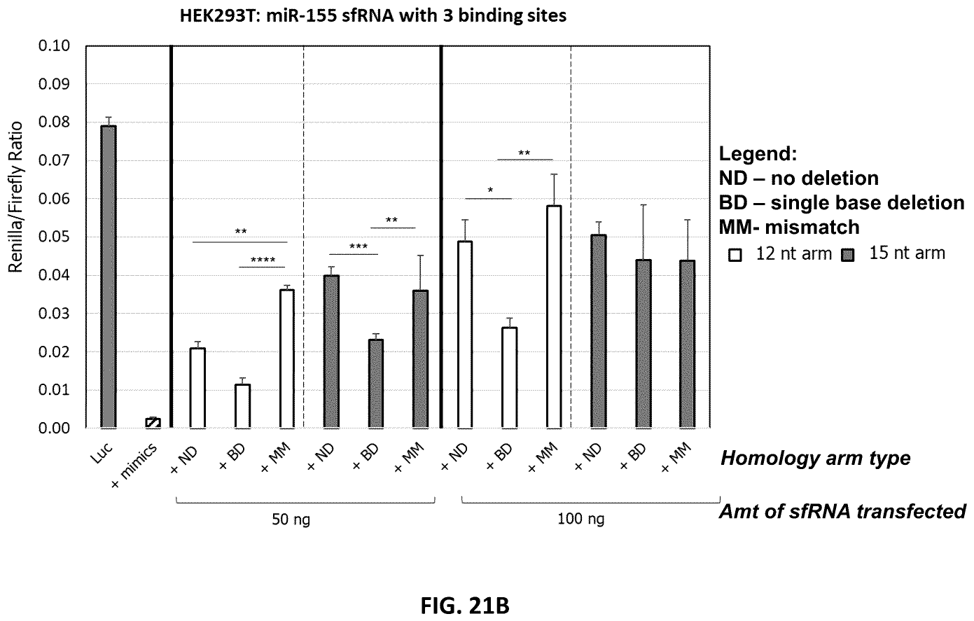

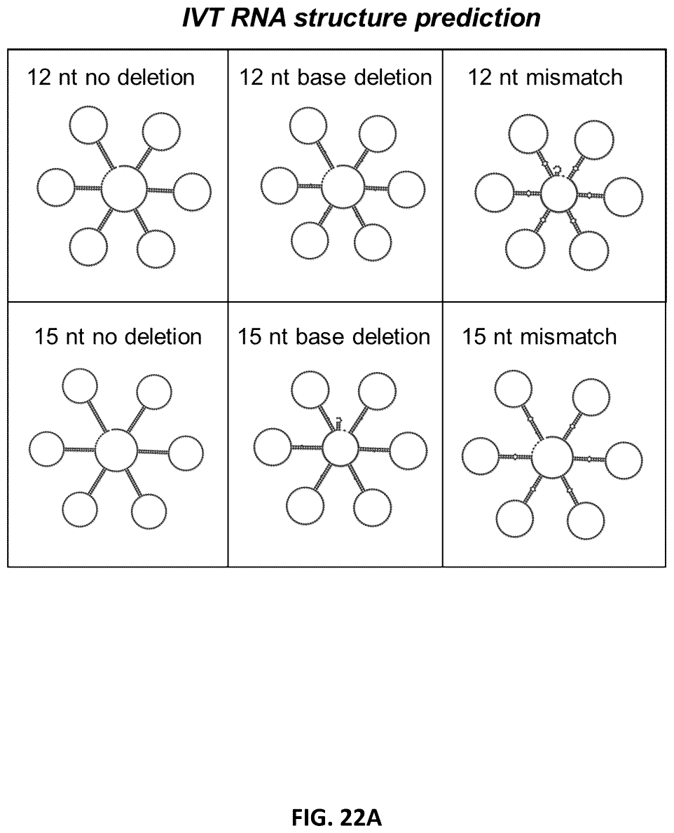

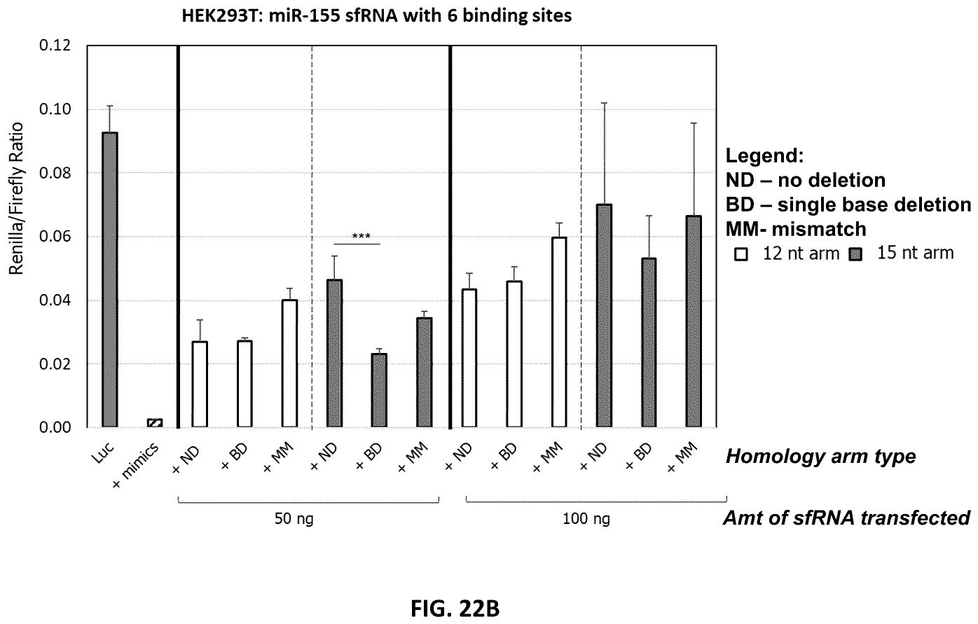

Engineered RNAs were tested with varying stem sizes (of stem loop, i.e., homology arm) sizes and structure modifications. 12 nucleotide (nt) and 15 nt homology arm sizes containing no deletions (ND), a single base deletion in one strand of an homology arm (BD), or a mismatch sequence in the homology arm (MM) were tested in the sfRNAs with 3 and 6 binding sites as shown in FIG. 21A and FIG. 22A. The engineered RNAs were tested for their ability to bind and sponge miR-155 mimics. Briefly, the functional efficacy of the different homology arm sizes in an engineered polynucleotide was validated in a dual-luciferase reporter system assay described above.

HEK293T cells were transfected with 25 ng luciferase vector, 0.5 pmol of miR-155 mimics and various doses (50 or 100 ng) of in vitro T7 synthesized miR-155 sfRNA with different homology arm type (12 or 15 nt with ND, BD or MM) using Lipofectamine 2000 reagent. The luciferase assay was completed 48 hours post transfection.

FIG. 21B shows the luciferase results for the 3 site sfRNA with different sized homology arms. FIG. 21B shows that sfRNAs carrying 12 nt BD homology arms showed a reduced luciferase rescue effect compared to sfRNAs carrying 12 nt MM or ND homology arms at both 50 and 100 ng. sfRNAs carrying 15 nt BD homology arms also showed a reduced functional effect compared to 15 nt MM or ND homology arms at 50 ng. MM homology arms exerted a stronger effect than ND homology arms for 12 nt but not 15 nt at 50 ng.

FIG. 22B shows the luciferase results for the 6 site sfRNA with different sized homology arms. FIG. 22B shows that 12 nt ND, BD or MM homology arms showed similar effects across the different doses. Similar trend was observed with 15 nt homology arms with the exception of BD homology arms showing a reduced rescue effect compared to ND homology arms at 50 ng.

### Example 8: Determining Binding Site Type in sfRNA Targeting miR-155

Engineered RNAs were tested with varying binding site types as described above. The engineered RNAs were tested for their ability to bind miR-155 mimics. Briefly, the functional efficacy of binding site types in an engineered polynucleotide was validated in a dual-luciferase reporter system assay described above.

HEK293T cells were transfected with 25 ng luciferase vector. 0.5 pmol of miR-155 mimics and 100 ng of in vitro T7 synthesized sfRNAs with different binding site types using Lipofectamine 2000 reagent. The luciferase assay was completed 48 hours post transfection.

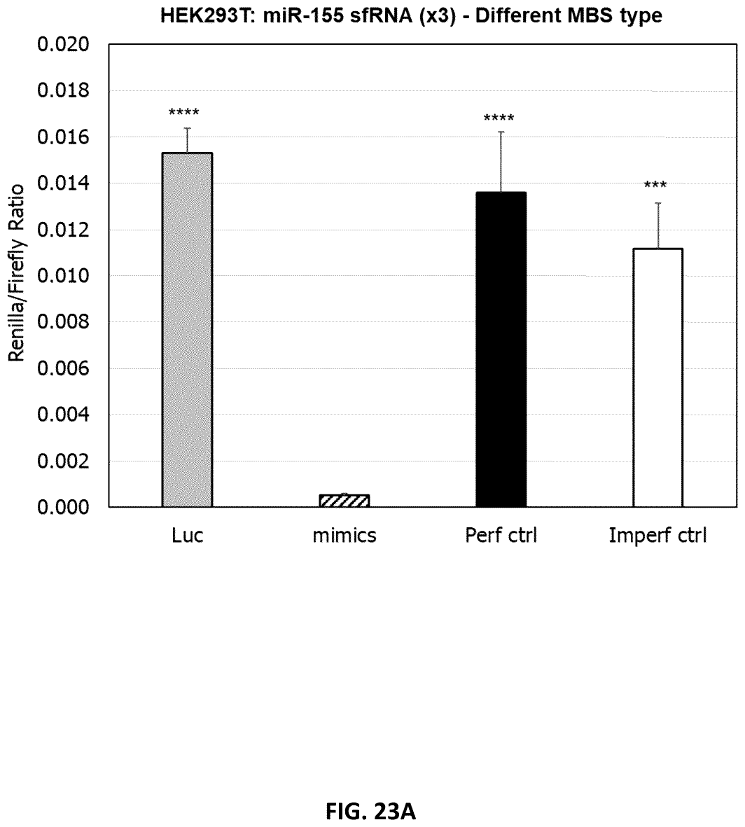

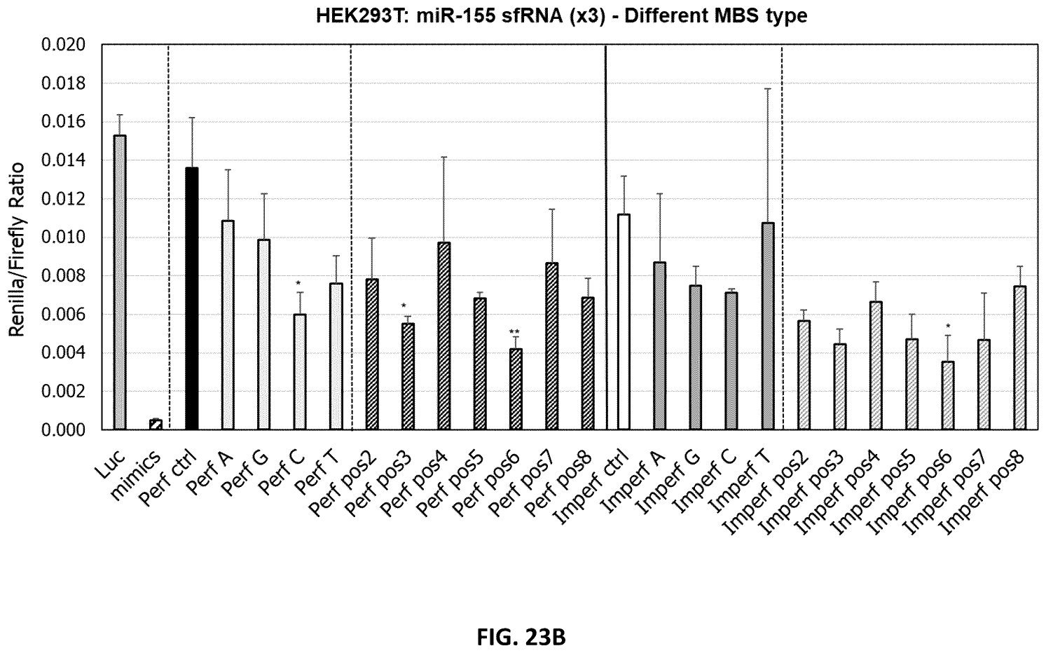

FIG. 23A-B shows the luciferase results for the 3 site sfRNA with different binding site types. Both perf and imperf ctrl binding sites showed a significant luciferase rescue effect (FIG. 23A). Findings indicate that certain seed mutations are as effective as the perfect seed region in ctrl (Perf/Imperf) binding sites. All mutations except Perf C. Perf pos3 and Perf pos6 showed comparable activity to Perf ctrl binding site (FIG. 23B). All mutations except Imperf pos6 showed comparable activity to Imperf ctrl binding site (FIG. 23B).

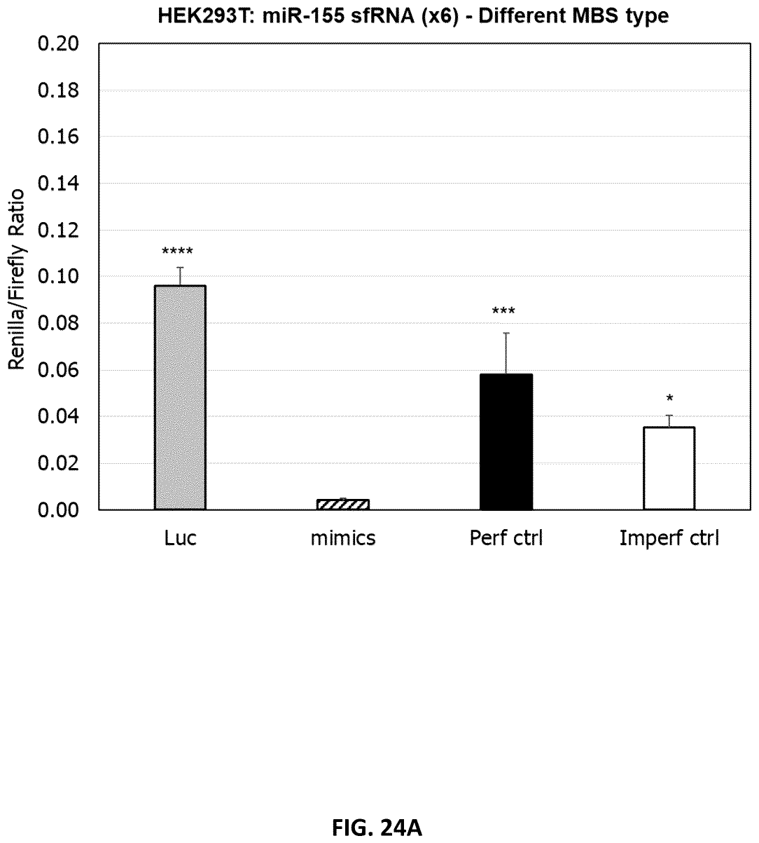

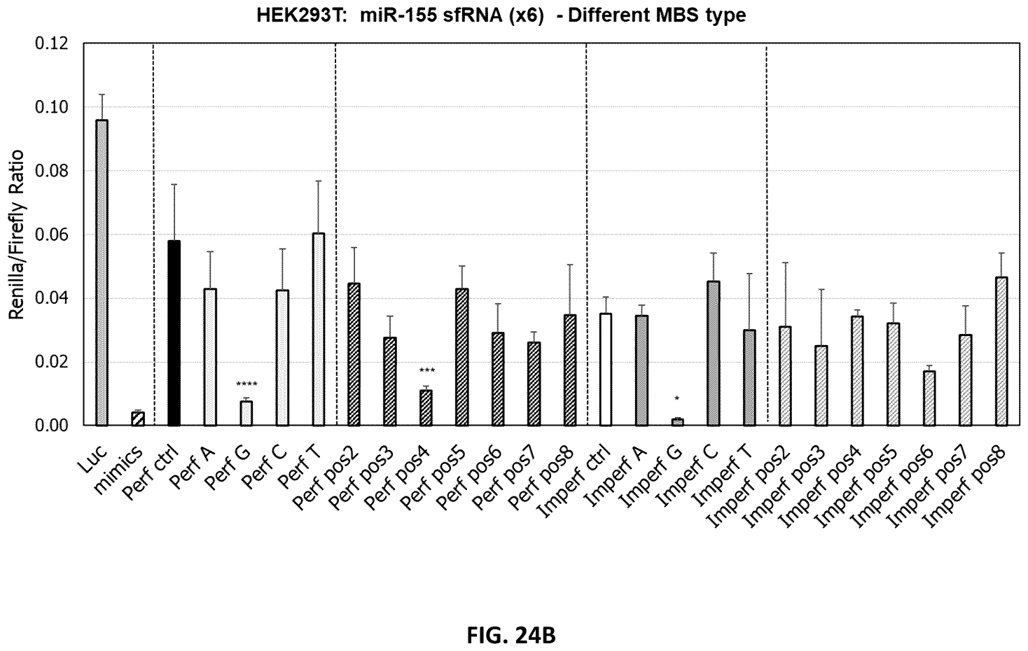

FIG. 24A-B shows the luciferase results for the 6 site sfRNA with different binding site types. Both perf and imperf ctrl binding sites showed a significant luciferase rescue effect (FIG. 24A). Imperf ctrl sfRNA showed a weaker rescue effect than perf ctrl sfRNA, although this difference was not statistically significant (FIG. 24A). All mutations except Perf G and Perf pos4 showed comparable activity to Perf ctrl binding site (FIG. 24B). All mutations except Imperf G showed comparable activity to Imperf ctrl binding site (FIG. 24B).

### Example 9: Determining Auxiliary Arm Length in sfRNA Targeting miR-155

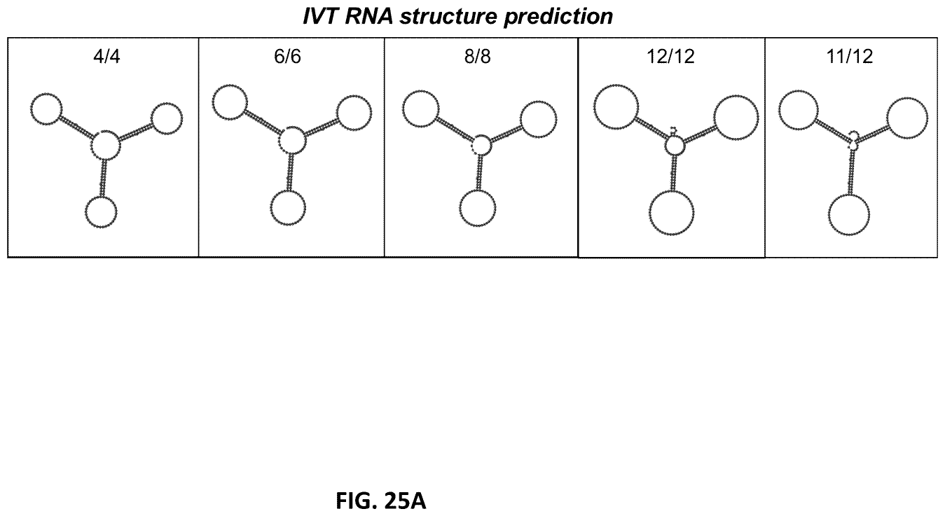

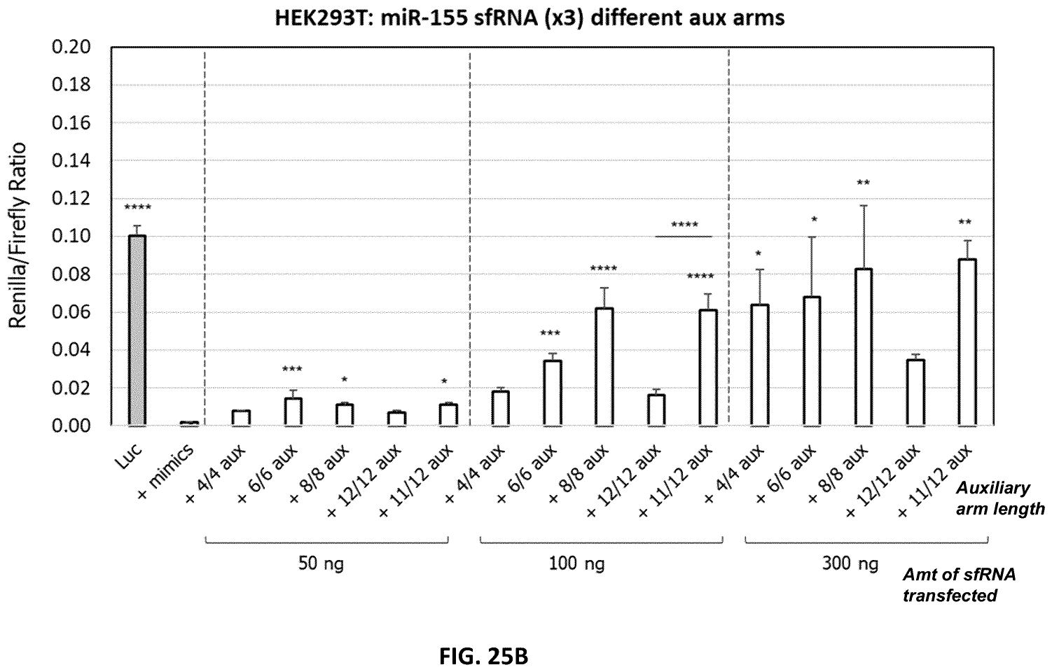

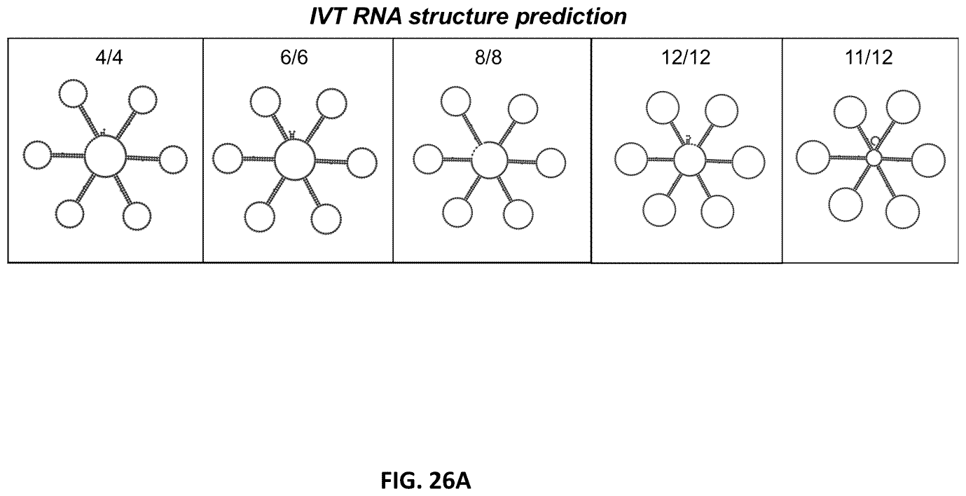

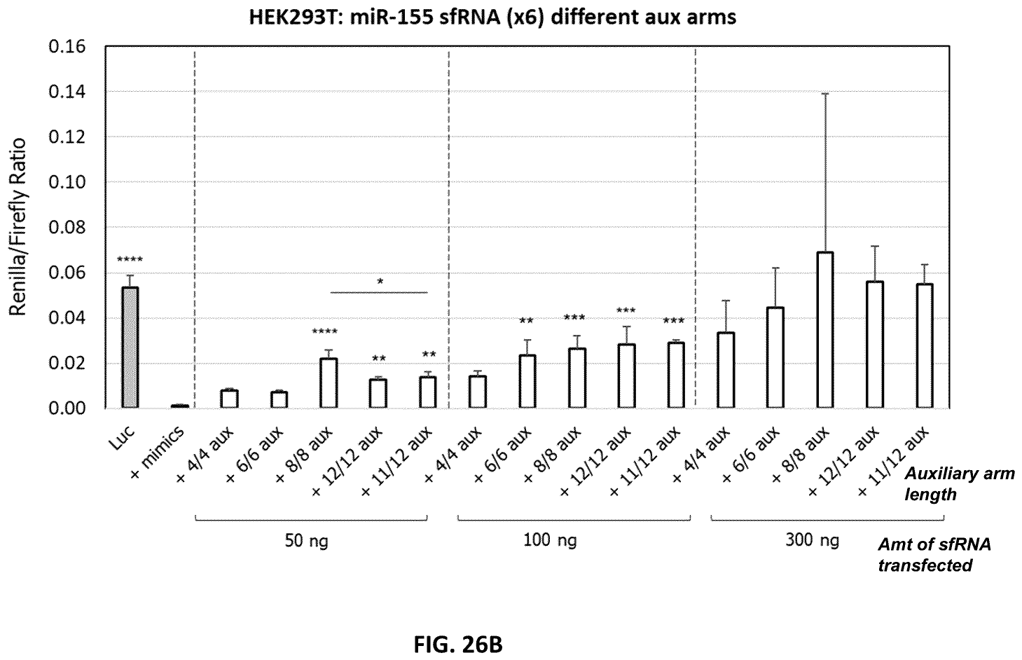

Engineered RNAs were tested with varying auxiliary arm lengths. The following auxiliary arm lengths: 4 nucleotide (nt) pair (4/4). 6 nt pair (6/6). 8 nt pair (8/8). 12 nt pair (12/12). 11 nt and 12 nt pair (11/12) flanking the binding site were tested in sfRNAs with 3 and 6 binding sites as shown in FIG. 25A and FIG. 26A. The engineered RNAs were tested for their ability to bind miR-155 mimics. Briefly, the functional efficacy of the auxiliary arm lengths in an engineered polynucleotide was validated in a dual-luciferase reporter system assay described above.

HEK293T cells were transfected with 25 ng luciferase vector, 0.5 pmol of miR-155 mimics and various doses (50, 100 or 300 ng) of in vitro T7 synthesized sfRNAs with different auxiliary arm lengths (4/4, 6/6, 8/8, 12/12, 11/12 nt) using Lipofectamine 2000 reagent. The luciferase assay was completed 48 hours post transfection.

FIG. 25B shows the luciferase results for sfRNA with different auxiliary arm lengths for 3 binding sites. Luciferase rescue effect increased progressively with increasing auxiliary arm length from 4 to 6 to 8 nt (FIG. 25B). However, the effect dropped by 3-fold when auxiliary arm length increased from 8 to 12 nt (FIG. 25B). A single nt deletion resulting in a 11 nt and 12 nt auxiliary arm pair improved function similar to 8 nt auxiliary arms (FIG. 25B). These differences were noted at 100 and 300 ng doses, whereas 50 ng was too low to detect any significant changes.

FIG. 26B shows the luciferase results for sfRNA with different auxiliary arm lengths for 6 binding sites. sfRNA carrying auxiliary arm lengths of 8 nt showed better function compared to 11/12 nt auxiliary arms at 50 ng (FIG. 26B). No significant changes across different auxiliary arm lengths were observed at 100 or 300 ng (FIG. 26B).

### Example 10: Determining the Number of Binding Sites in an Engineered sfRNA Targeting miR-132

sfRNAs were tested with varying numbers of imperf miR-132 binding sites. The engineered RNAs were tested for their ability to bind miR-132 mimics and rescue luciferase activity. Briefly, the functional efficacy of the number of binding sites in an engineered polynucleotide was validated in a luciferase rescue assay. miRNA binding sites (MBS) for miR-132 was inserted into the 3′-UTR of a Renilla luciferase control construct from a dual-luciferase reporter system.

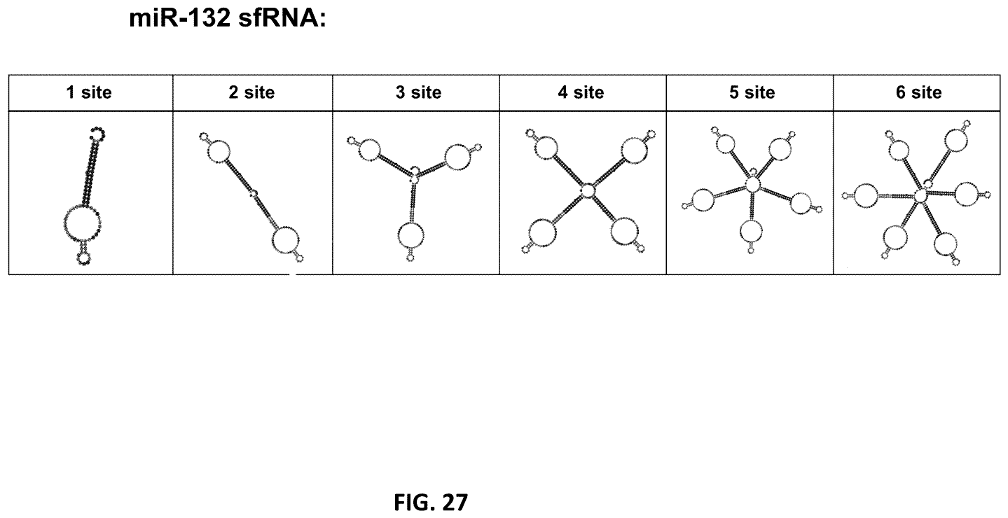

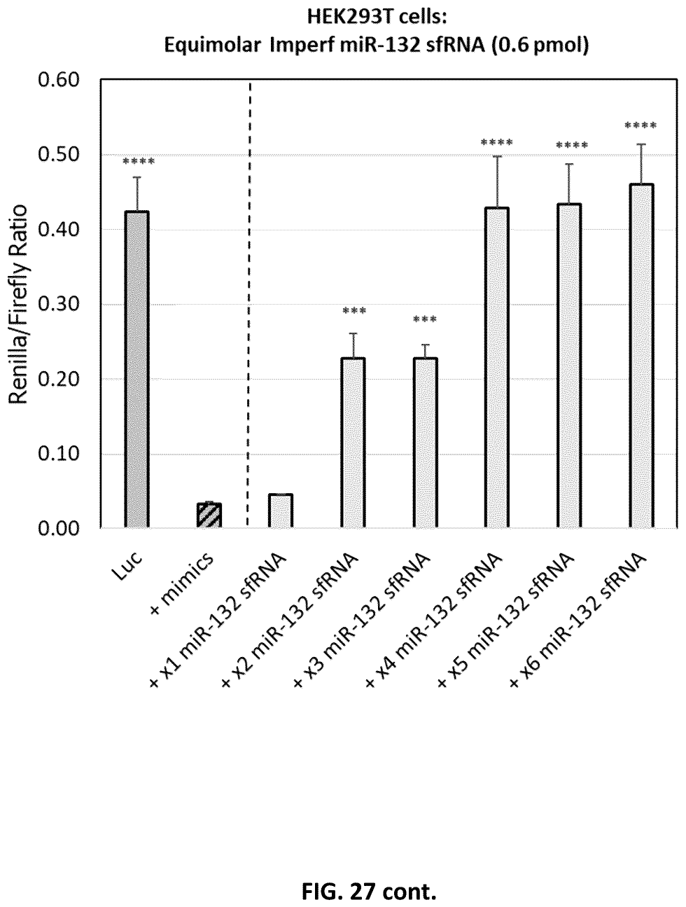

HEK293T cells were co-transfected with reporter plasmids, miR-132 mimics and/or respective engineered polynucleotides (snowflake RNA-sfRNA) targeting the miR-132 mimics for 48 h at 0.6 pmol (equimolar) concentrations to determine the effect of the number of binding sites on the sfRNA. The sfRNA was tested with 1, 2. 3, 4, 5, and 6 binding sites as shown in FIG. 27. Also FIG. 27, shows that 4, 5 or 6 binding sites on a sfRNA were the most effective in sponging and reducing the effect of miR-132 on luciferase expression.

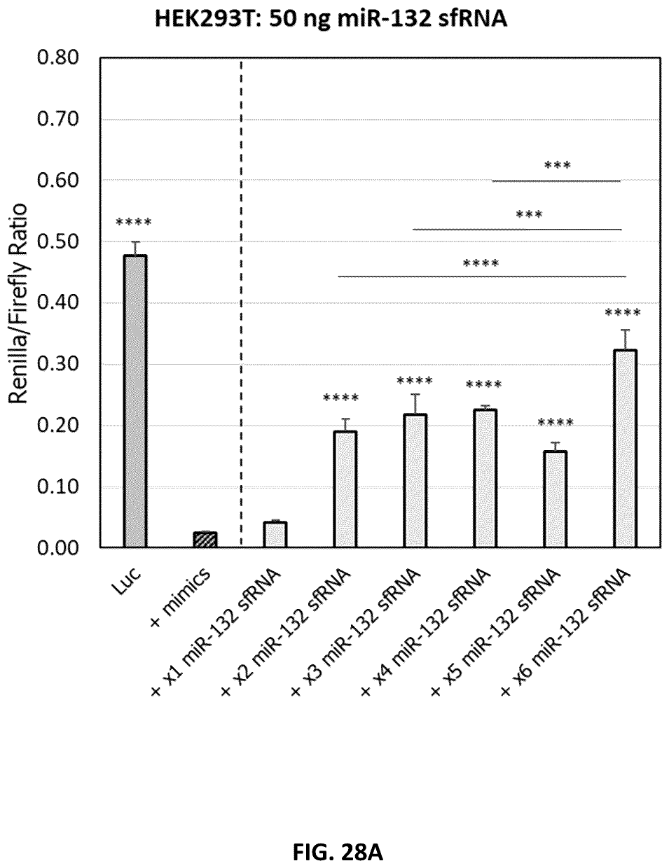

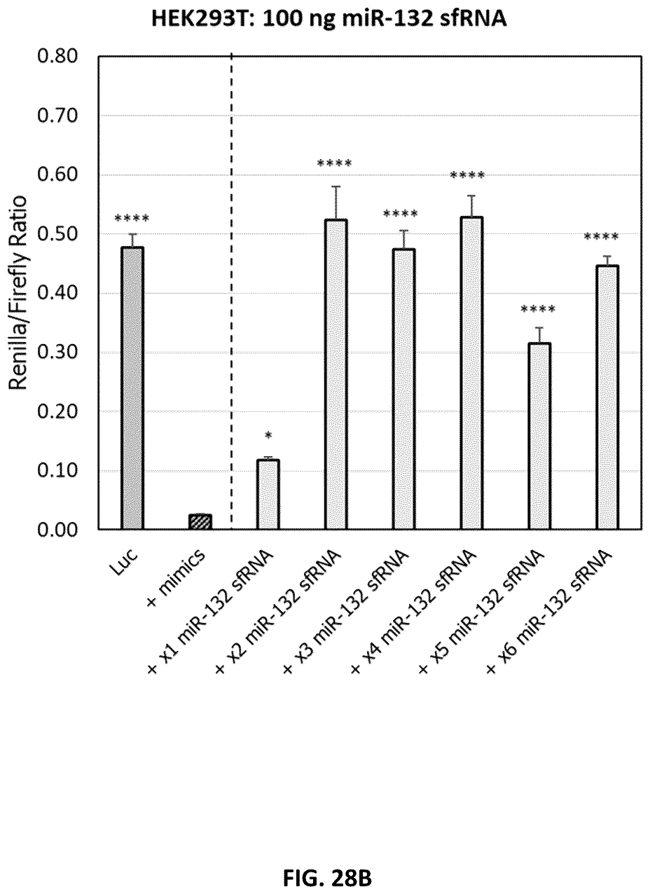

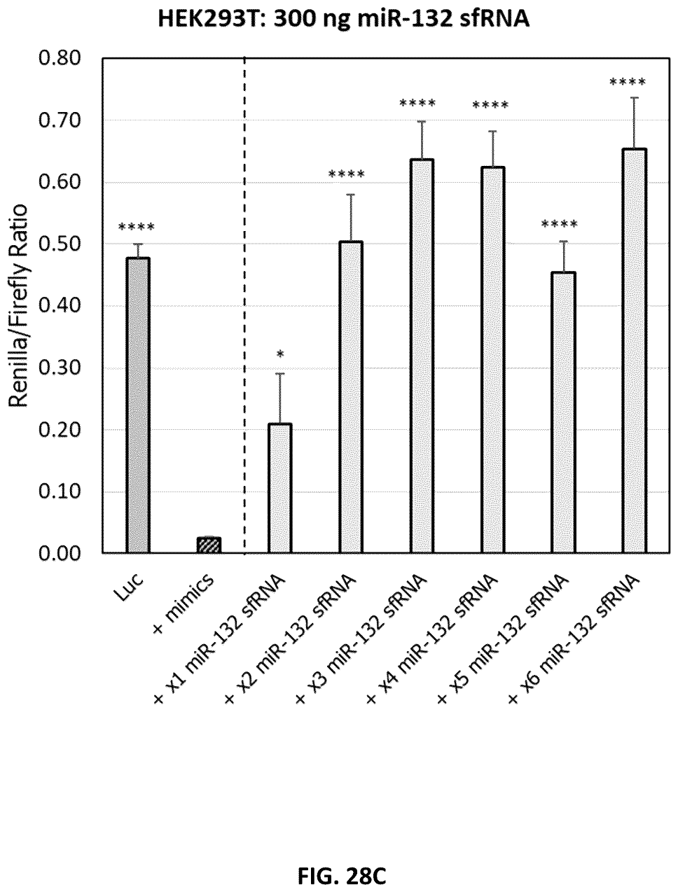

Additionally, an equal number of binding sites presented by sfRNAs with varying numbers of binding sites were tested in their ability to sponge miR-155 in the dual-luciferase reporter system. The following molar ratios of sfRNA were transfected at either 50, 100 or 300 ng to achieve a total number of 60 binding sites: 60 moles of 1 binding site, 30 moles of 2 binding sites, 20 moles of 3 binding sites, 15 moles of 4 binding sites, 12 moles of 5 binding sites, and 10 moles of 6 binding sites as shown in FIG. 18A. FIGS. 28A-C shows that across all three doses (FIG. 28A—50 ng; FIG. 28B—100 ng, FIG. 28C—300 ng), 1 and 5 binding sites consistently displayed a weaker functional effect. While 6 binding sites showed the most effective rescue followed by 2, 3 and 4 sites at 50 ng, similar effect was observed across 2, 3, 4 and 6 sites at 100 and 300 ng.

### Example 11: Determining Linker Size in sfRNA Targeting miR-132

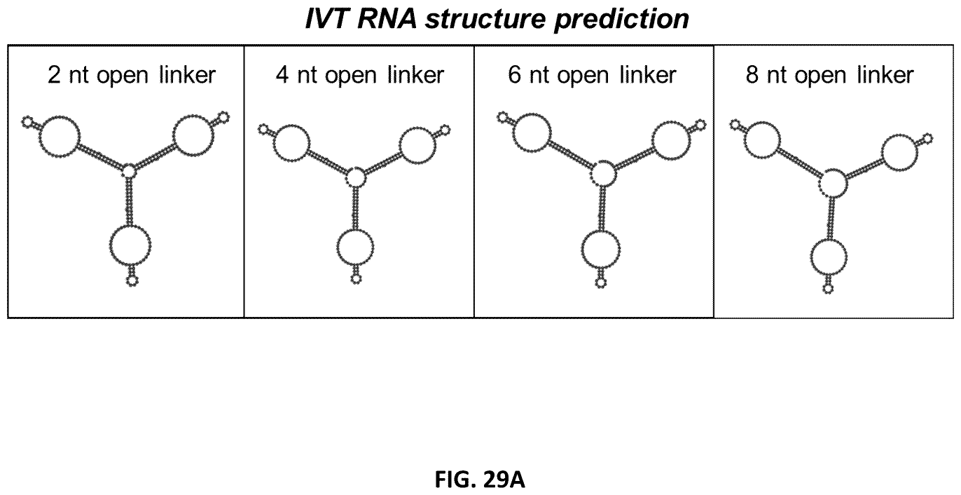

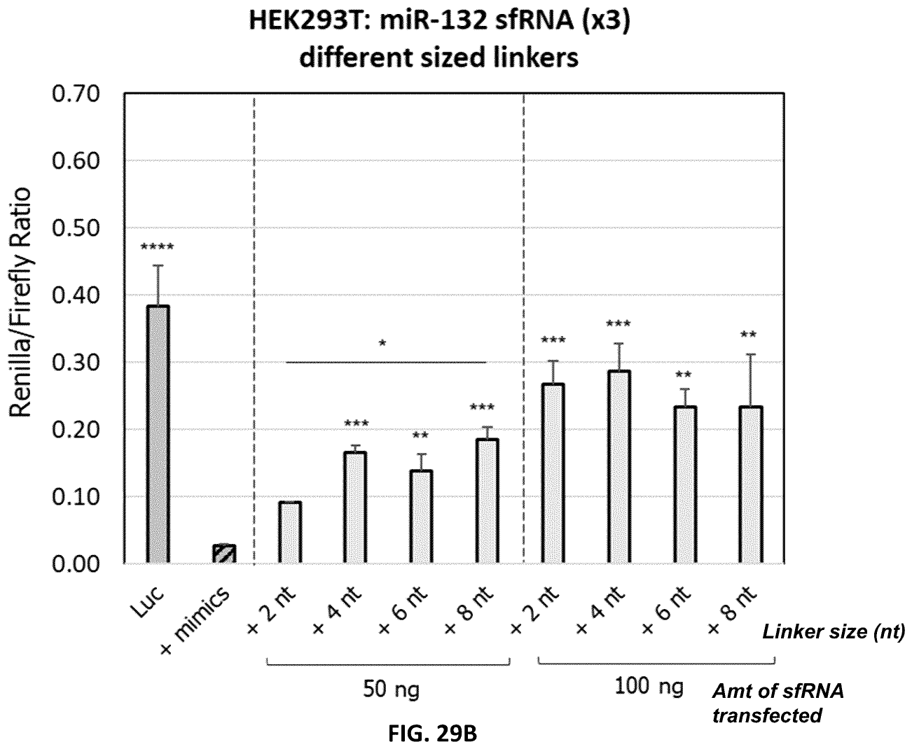

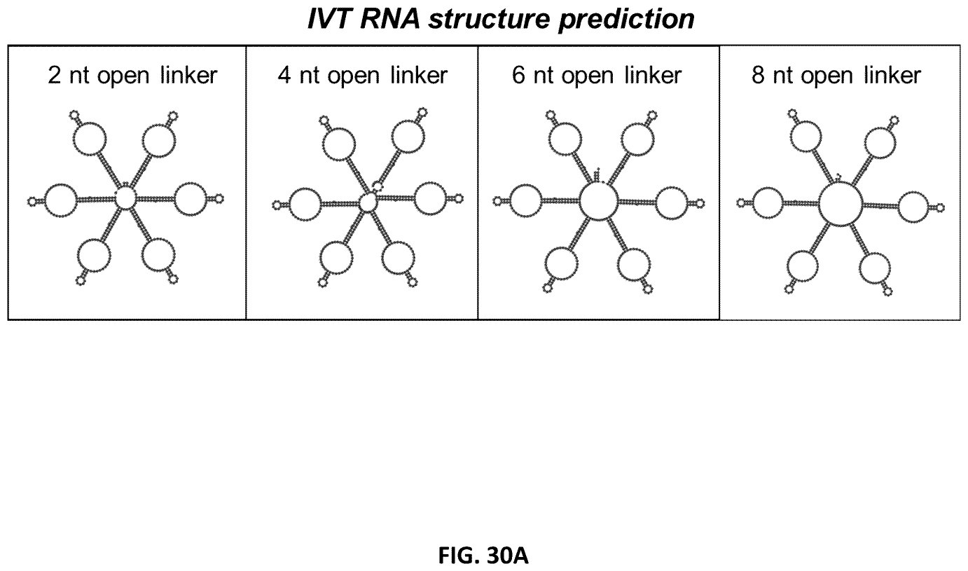

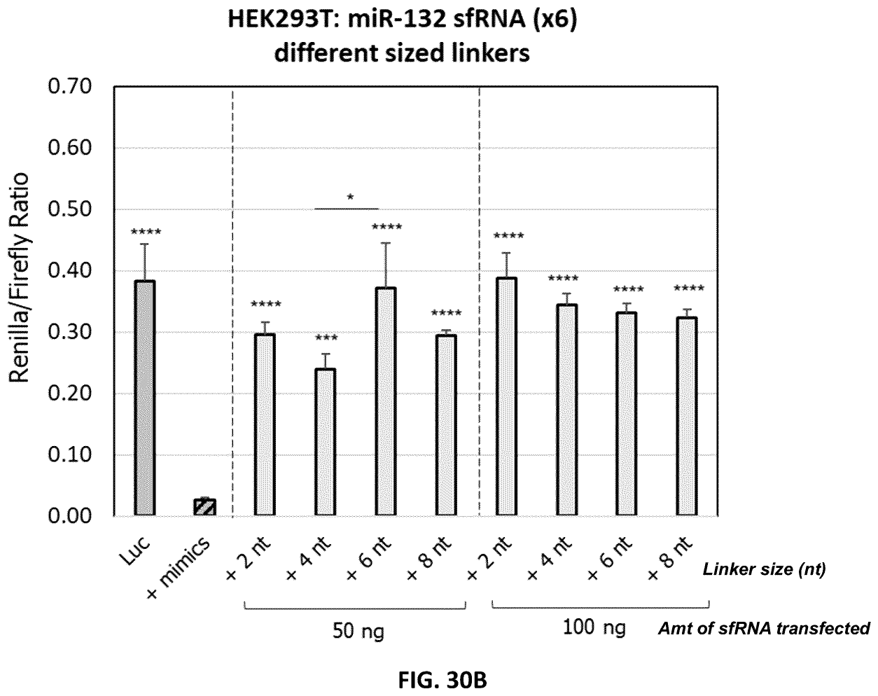

Engineered RNAs were tested with varying linker sizes. 2 nucleotide (nt), 4 nt, 6 nt, and 8 nt linker sizes between stem loops were tested in sfRNAs with 3 and 6 binding sites as shown in FIG. 29A and FIG. 30A. The engineered RNAs were tested for their ability to bind miR-132 mimics. Briefly, the functional efficacy of the linker sizes in an engineered polynucleotide was validated in a dual-luciferase reporter system assay described above.

HEK293T cells were transfected with 25 ng luciferase vector, 0.5 pmol of miR-132 mimics and various doses (50 or 100 ng) of in vitro T7 synthesized sfRNAs with different sized linkers (2, 4, 6, 8 nt) using Lipofectamine 2000 reagent. The luciferase assay was completed 48 hours post transfection. FIG. 29B and FIG. 30B shows luciferase results for sfRNA with different sized linkers for 3 and 6 binding sites respectively.

3 site sfRNA with 8 nt linkers were more effective than 2 nt linkers at 50 ng dose (FIG. 29B). 6 site sfRNA with 6 nt linkers were more effective than 4 nt linkers at 50 ng dose (FIG. 30B). No significant changes across different sized linkers were observed at 100 ng doses for both 3 and 6 site sfRNA (FIGS. 29B and 30B).

### Example 12: Determining Stem Design in sfRNA Targeting miR-132

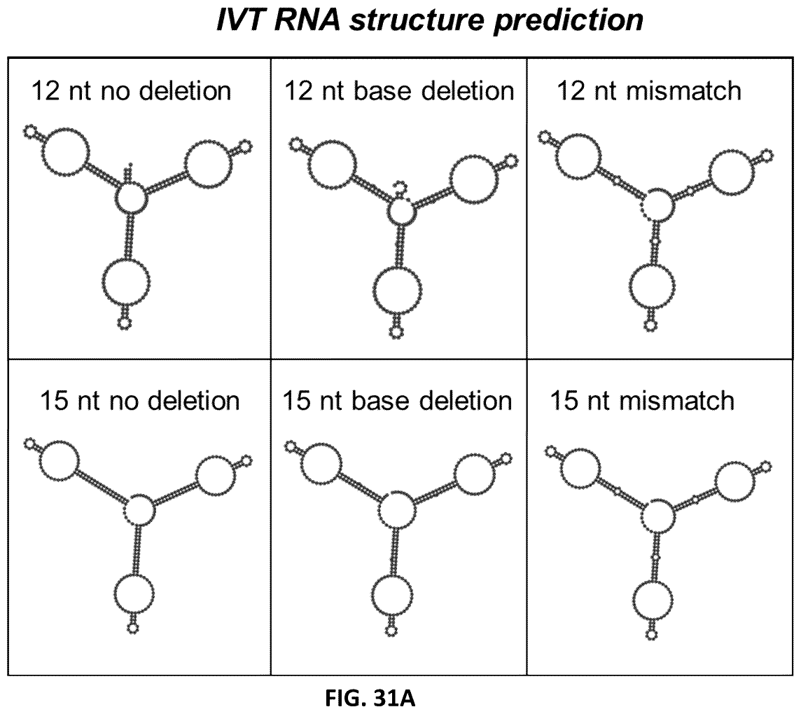

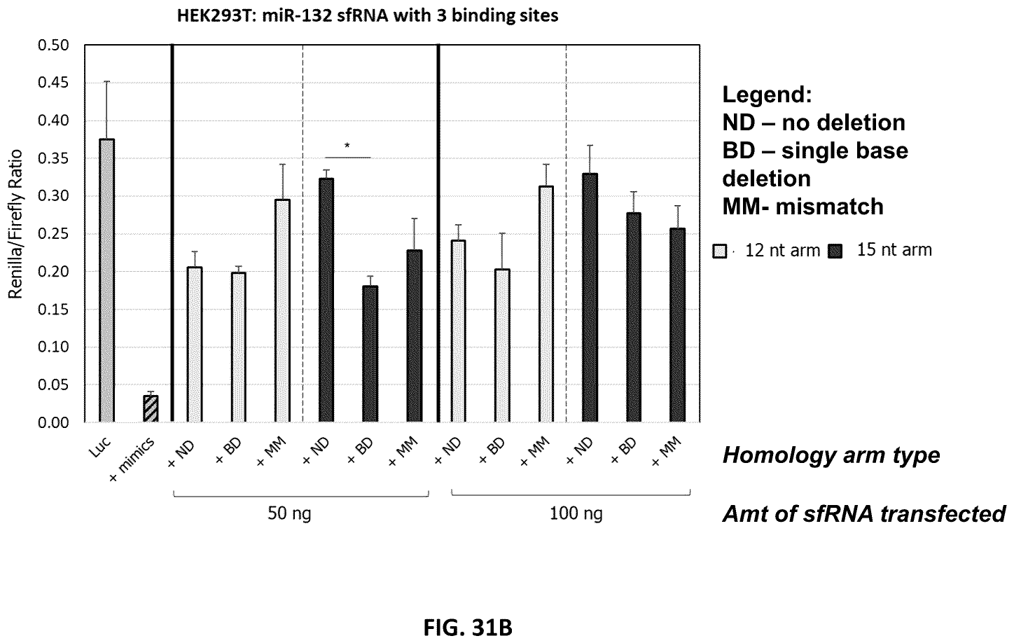

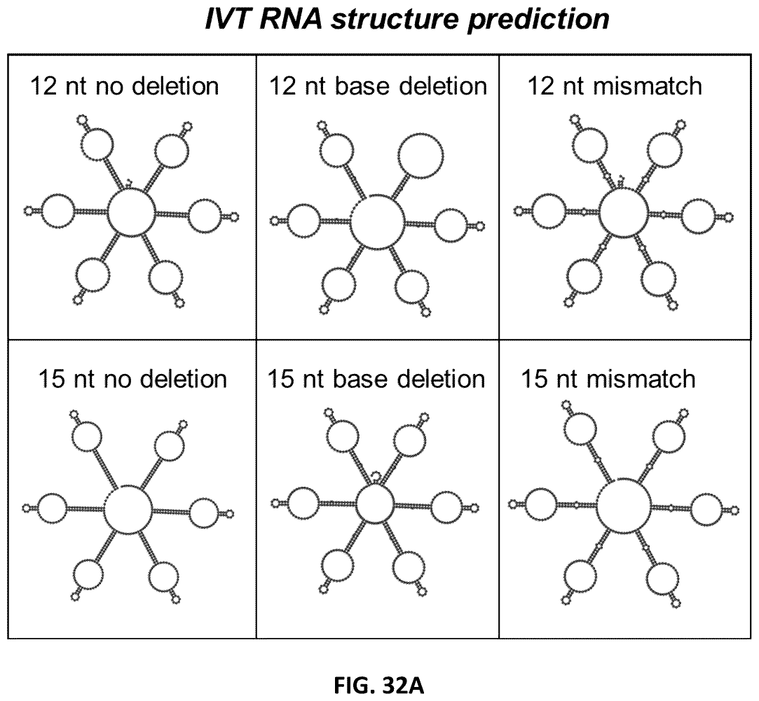

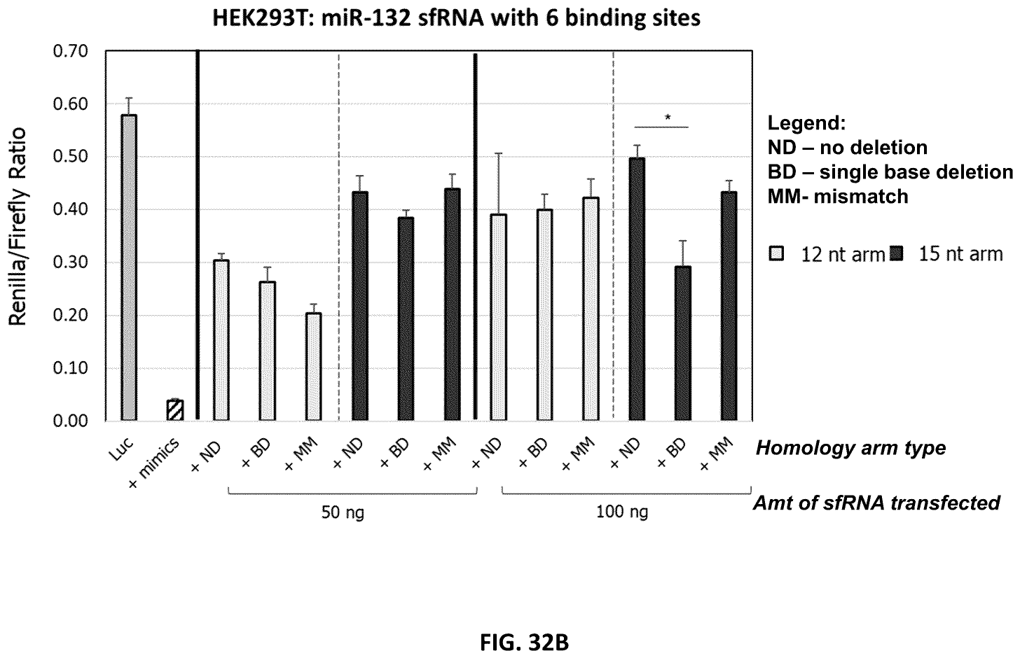

Engineered RNAs were tested with varying stem sizes (of stem loop, i.e., homology arm) sizes and structure modifications. 12 nucleotide (nt) and 15 nt homology arm sizes containing no deletions (ND), a single base deletion in one strand of an homology arm (BD), or a mismatch sequence in the homology arm (MM) were tested in the sfRNAs with 3 and 6 binding sites as shown in FIG. 31A and FIG. 32A. The engineered RNAs were tested for their ability to bind and sponge miR-132 mimics. Briefly, the functional efficacy of the different homology arm sizes in an engineered polynucleotide was validated in a dual-luciferase reporter system assay described above.

HEK293T cells were transfected with 25 ng luciferase vector, 0.5 pmol of miR-132 mimics and various doses (50 or 100 ng) of in vitro T7 synthesized miR-132 sfRNA with different homology arm type (12 or 15 nt with ND, BD or MM) using Lipofectamine 2000 reagent. The luciferase assay was completed 48 hours post transfection.

FIG. 31B shows the luciferase results for 3 site sfRNA with different sized homology arms. At 50 and 100 ng, sfRNAs carrying 12 nt MM homology arms showed greater luciferase rescue effect compared to 12 nt ND or BD homology arms although this difference was not significant. At 50 ng, sfRNA carrying 15 nt ND arms showed greater luciferase effect compared to 15 nt BD arms although this difference was not observed at a higher dose of 100 ng at which effect might be saturating.

FIG. 32B shows the luciferase results for 6 site sfRNA with different sized homology arms. While no significant differences were observed among different 15 nt arm types at 50 ng. 15 nt ND homology arms showed better rescue than 15 nt BD arms at 100 ng (FIG. 32B). Similar effect was observed across 12 nt ND, BD or MM homology arms at different doses (FIG. 32B).

### Example 13: Determining Binding Site Type in sfRNA Targeting miR-132

Engineered RNAs were tested with varying binding site types as described above. The engineered RNAs were tested for their ability to bind miR-132 mimics. Briefly, the functional efficacy of binding site types in an engineered polynucleotide was validated in a dual-luciferase reporter system assay described above.

HEK293T cells were transfected with 25 ng luciferase vector, 0.5 pmol of miR-132 mimics and 100 ng of in vitro T7 synthesized sfRNAs with different binding site types using Lipofectamine 2000 reagent. The luciferase assay was completed 48 hours post transfection.

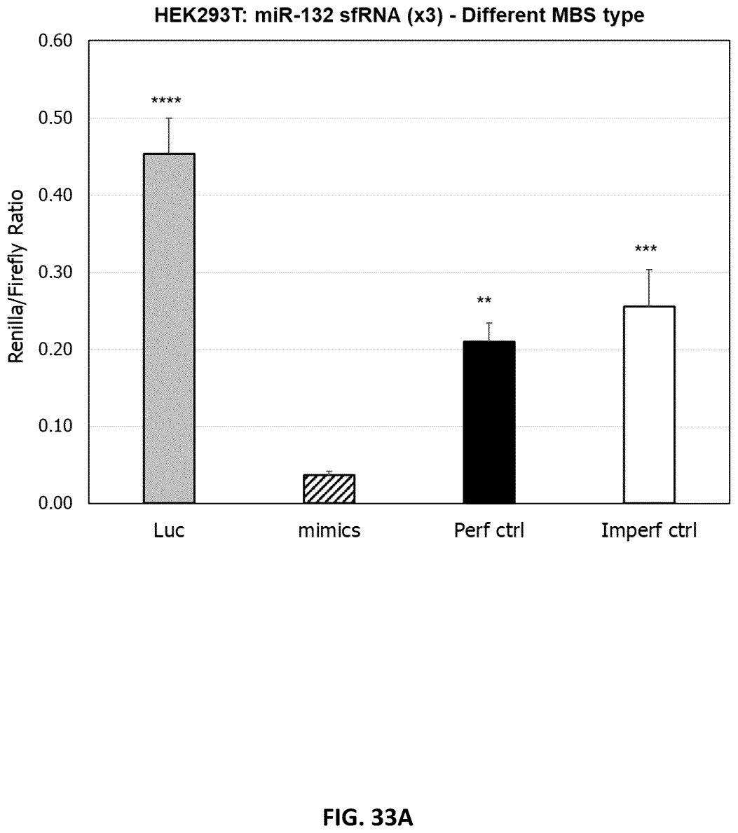

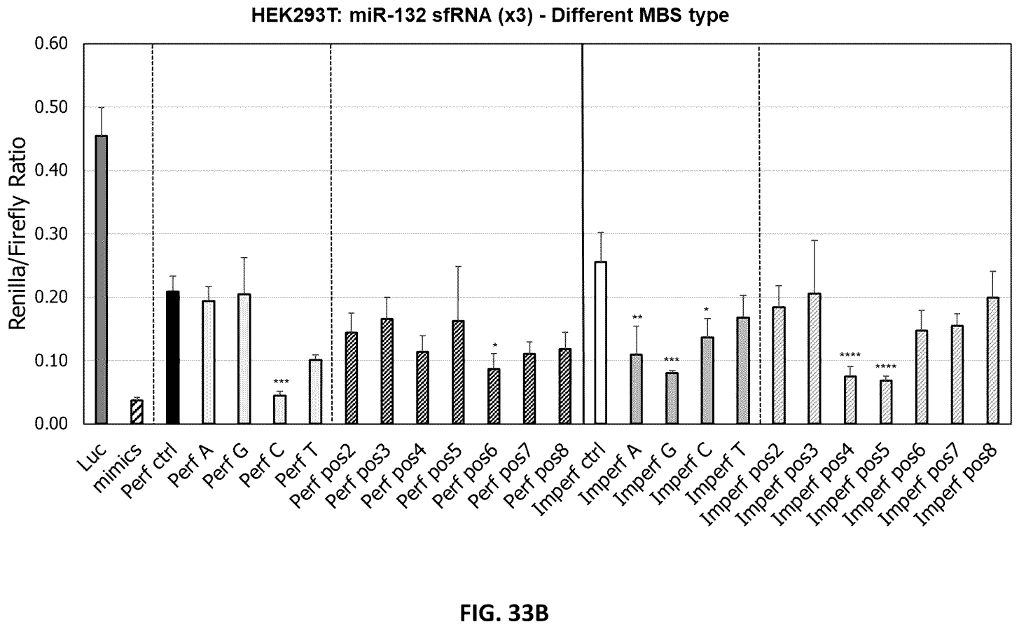

FIGS. 33A-B shows the luciferase results for the 3 site sfRNA with different binding site types. Both perf and imperf ctrl binding sites showed a significant luciferase rescue effect in which no difference was observed between the two. (FIG. 33A). Findings indicate that certain seed mutations are as effective as the perfect seed region in ctrl (Perf/Imperf) binding sites. All mutations except Perf C and Perf pos6 showed comparable activity to Perf ctrl binding site (FIG. 33B). All mutations except Imperf A. Imperf G, Imperf C, Imperf pos4 and Imperf pos5 showed comparable activity to Imperf ctrl binding site (FIG. 33B).

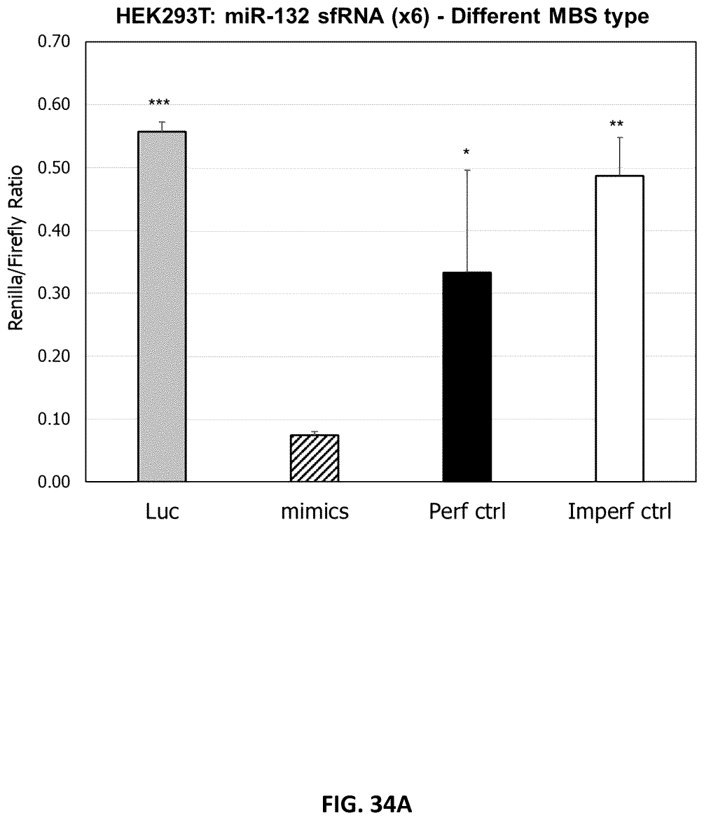

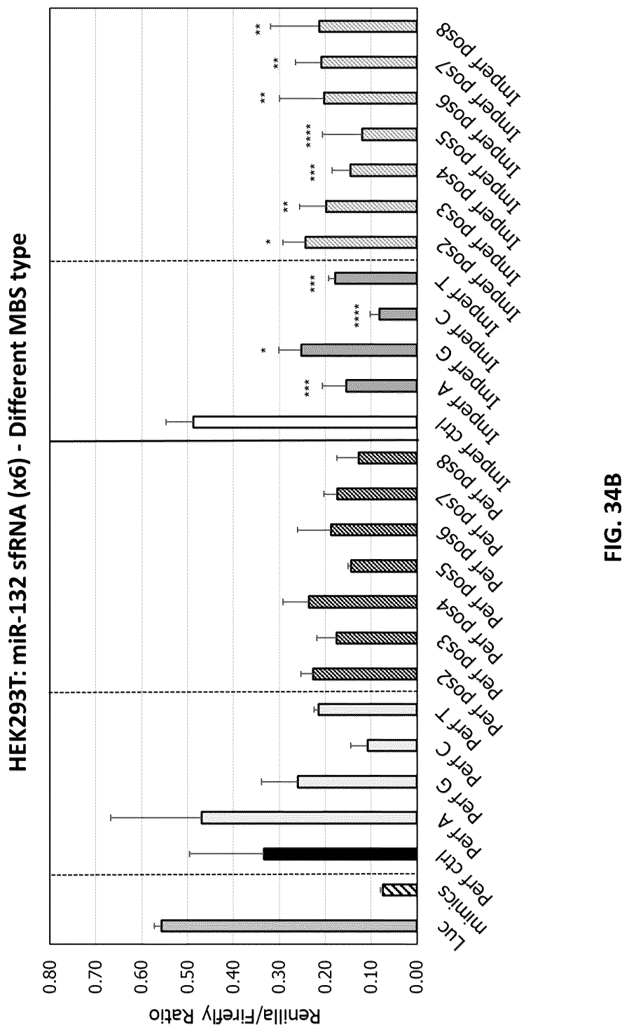

FIGS. 34A-B shows the luciferase results for the 6 site sfRNA with different binding site types. Both perf and imperf ctrl binding sites showed a significant luciferase rescue effect in which no difference was observed between the two (FIG. 34A). All perf mutations showed comparable activity to Perf ctrl binding site (FIG. 34B). On the contrary, all imperf mutations showed weaker activity compared to Imperf ctrl binding site (FIG. 34B).

### Example 14: Determining Auxiliary Arm Length in sfRNA Targeting miR-132

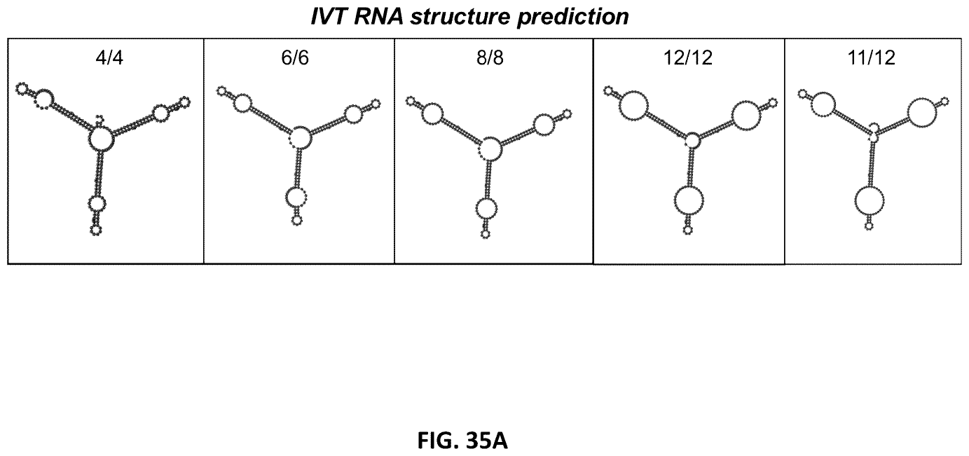

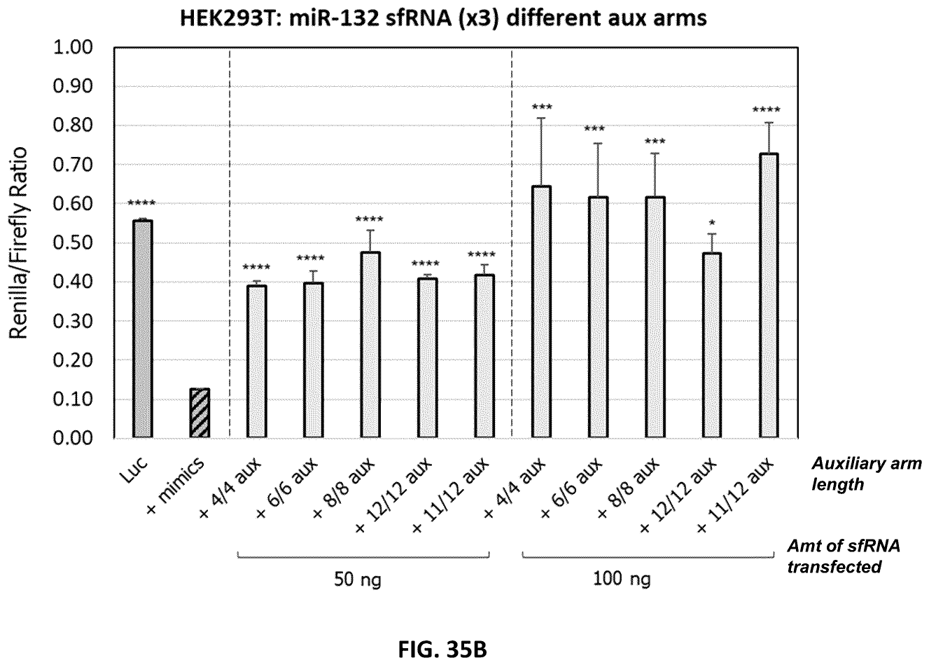

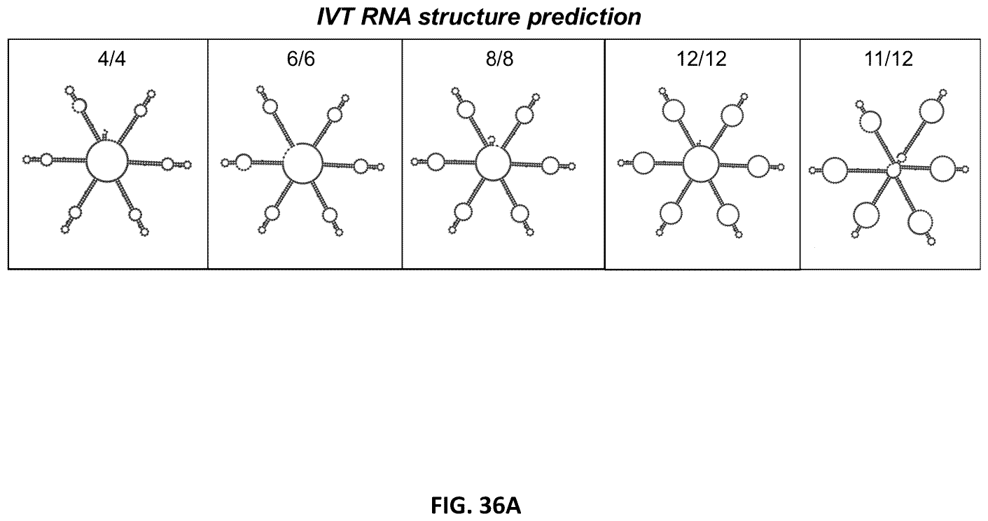

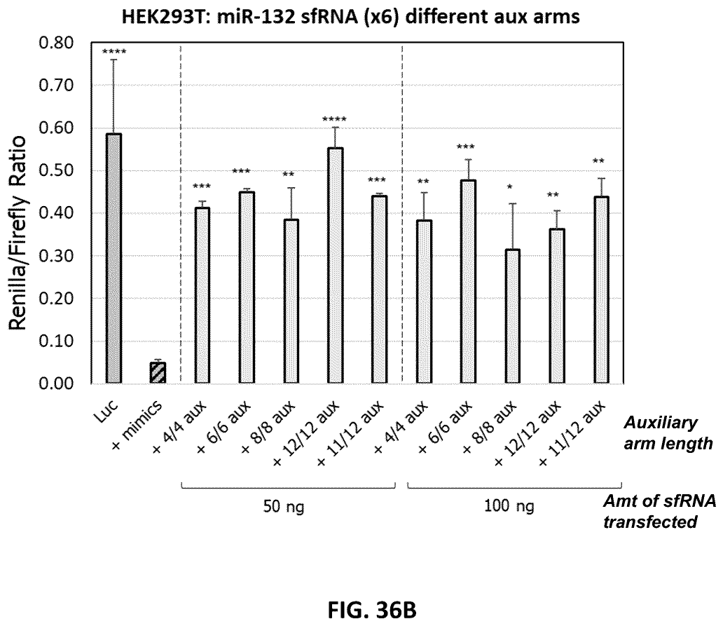

Engineered RNAs were tested with varying auxiliary arm lengths. The following auxiliary arm lengths: 4 nucleotide (nt) pair (4/4), 6 nt pair (6/6). 8 nt pair (8/8), 12 nt pair (12/12), 11 nt and 12 nt pair (11/12) flanking the binding site were tested in sfRNAs with 3 and 6 binding sites as shown in FIG. 35A and FIG. 36A. The engineered RNAs were tested for their ability to bind miR-132 mimics. Briefly, the functional efficacy of the auxiliary arm lengths in an engineered polynucleotide was validated in a dual-luciferase reporter system assay described above.

HEK293T cells were transfected with 25 ng luciferase vector, 0.5 pmol of miR-132 mimics and various doses (50 or 100 ng) of in vitro T7 synthesized sfRNAs with different auxiliary arm lengths (4/4, 6/6, 8/8, 12/12, 11/12 nt) using Lipofectamine 2000 reagent. The luciferase assay was completed 48 hours post transfection.

FIG. 35B shows the luciferase results for sfRNA with different auxiliary arm lengths for 3 binding sites. Luciferase rescue effect was similar across auxiliary arm lengths 4, 6, 8 12 and 11/12 nt with no significant differences across them (FIG. 35B).

FIG. 36B shows the luciferase results for sfRNA with different auxiliary arm lengths for 6 binding sites. Luciferase rescue effect was similar across auxiliary arm lengths 4, 6, 8. 12 and 11/12 nt (FIG. 36B).

### Example 15: sfRNA Targeting miR-155 with Modified Nucleotides Reduced Immunogenicity

A sfRNA targeting miR-155 engineered with complete or partial incorporation of modified nucleotides was produced. For modification substitution, the modified nucleotides, methylcytosine (5mCTP), pseudouridine (Ψ-UTP). N1-methylpseudouridine (me1Ψ-UTP) and m6A, were added in place of the standard unmodified nucleotides, cytosine, uridine and adenosine, respectively, during the in vitro transcription reaction.

Expression levels of innate immune response genes were measured in HEK293T cells transfected with unmodified or modified sfRNAs with either 5mCTP, Ψ-UTP, 5mCTP and Ψ-UTP, me1Ψ-UTP or m6A modifications. Total RNA was extracted using Trizol Reagent (Invitrogen) and subjected to reverse transcription to generate cDNA. qRT-PCR analysis for immune related genes was performed using SYBR green (Quantabio).

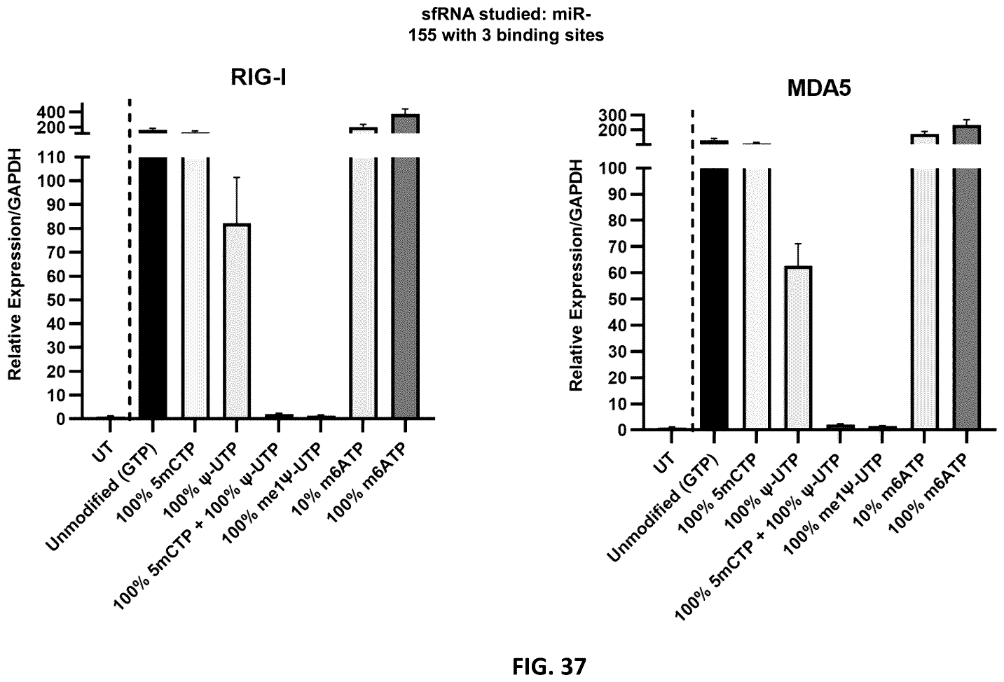

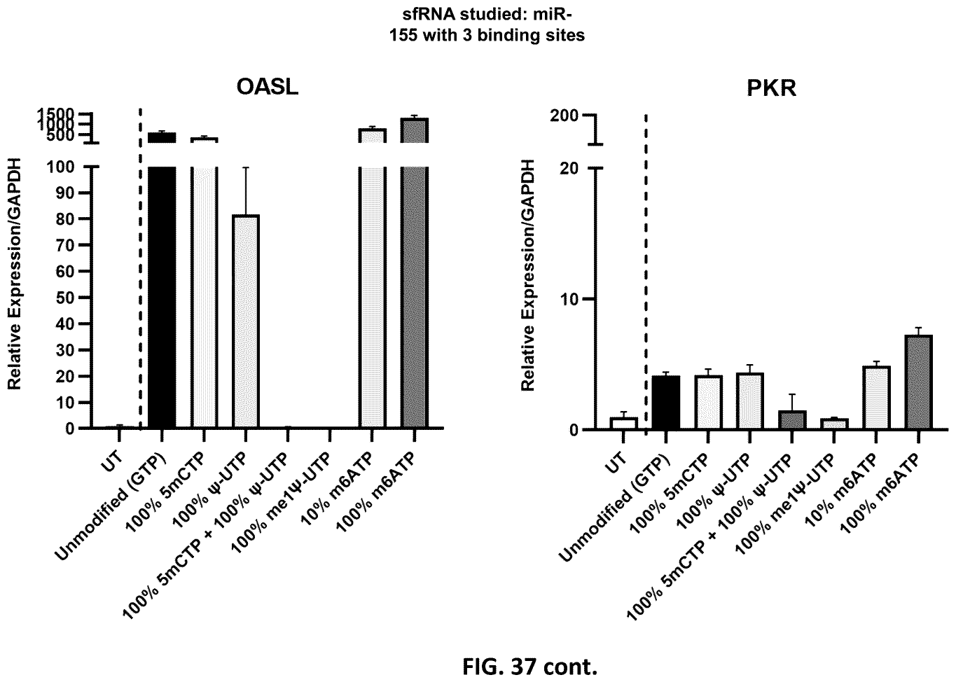

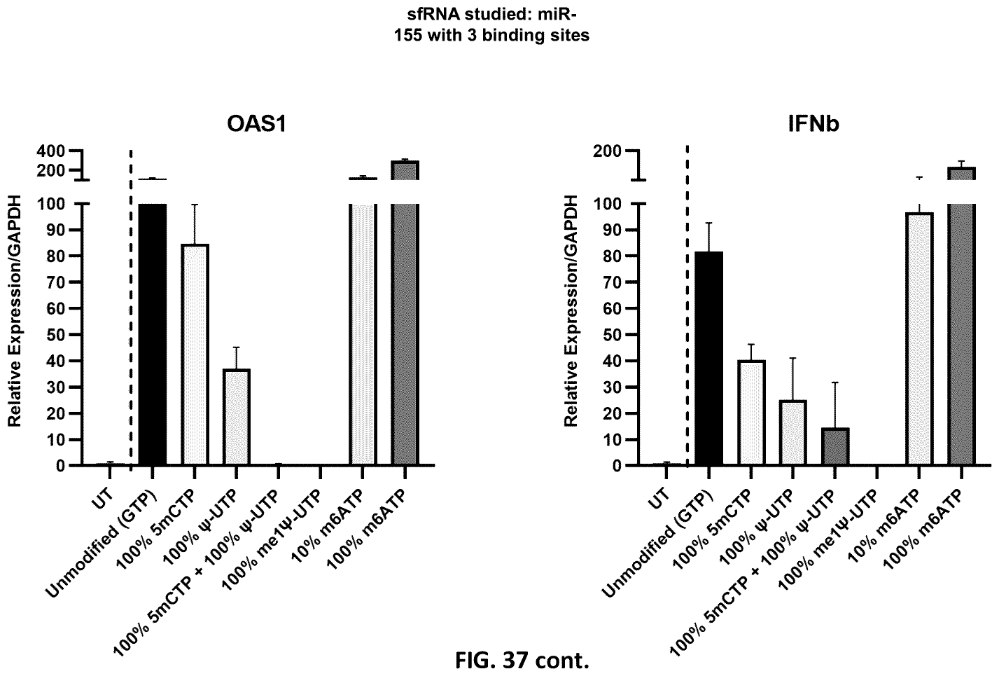

As shown in FIG. 37. 5mCTP and Ψ-UTP or me1Ψ-UTP modified sfRNAs showed the greatest reduction in the expression levels of RIG-I. MDA5. PKR. IFN-beta and OAS as compared to unmodified sfRNA. Ψ-UTP modified sfRNA showed a slight reduction in levels of immune response genes as compared to unmodified sfRNA (FIG. 37). 5mCTP and m6A modified sfRNA showed similar levels immune regulators expression as unmodified sfRNA (FIG. 37). Thus, the type of modification introduced in sfRNA influences activation of innate immune regulators.

### Example 16: sfRNA Targeting miR-155 with Modified Nucleotides and 5′ Monophosphate End Reduced Immunogenicity

A sfRNA targeting miR-155 engineered with a 5′ monophosphate end and modified nucleotides was produced. sfRNAs produced via in vitro transcription carry 5′ triphosphate (GTP) ends by convention. To synthesise sfRNAs with 5′ monophosphate (GMP) ends. GMP was added 10-fold in excess to GTP during the in vitro transcription reaction. For modification substitution, the modified nucleotides, methylcytosine (5mCTP), pseudouridine (Ψ-UTP), N1-methylpseudouridine (me1Ψ-UTP) and m6A, were added in place of the standard unmodified nucleotides, cytosine, uridine and adenosine, respectively, during the in vitro transcription reaction.

Expression levels of innate immune response genes were measured in HEK293T cells transfected with unmodified sfRNAs with 5′ GTP or 5′ GMP ends or modified sfRNAs with 5′ GMP ends carrying either 5mCTP. Ψ-UTP, 5mCTP and Ψ-UTP, me1Ψ-UTP or m6A modifications. Total RNA was extracted using Trizol Reagent (Invitrogen) and subjected to reverse transcription to generate cDNA. qRT-PCR analysis for immune related genes was performed using SYBR green (Quantabio).

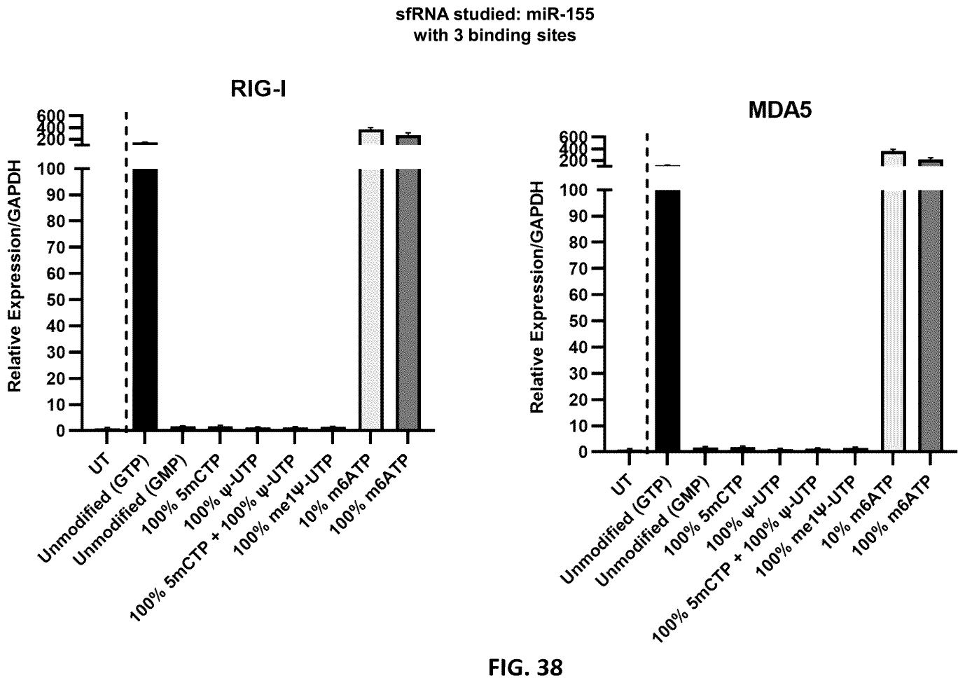

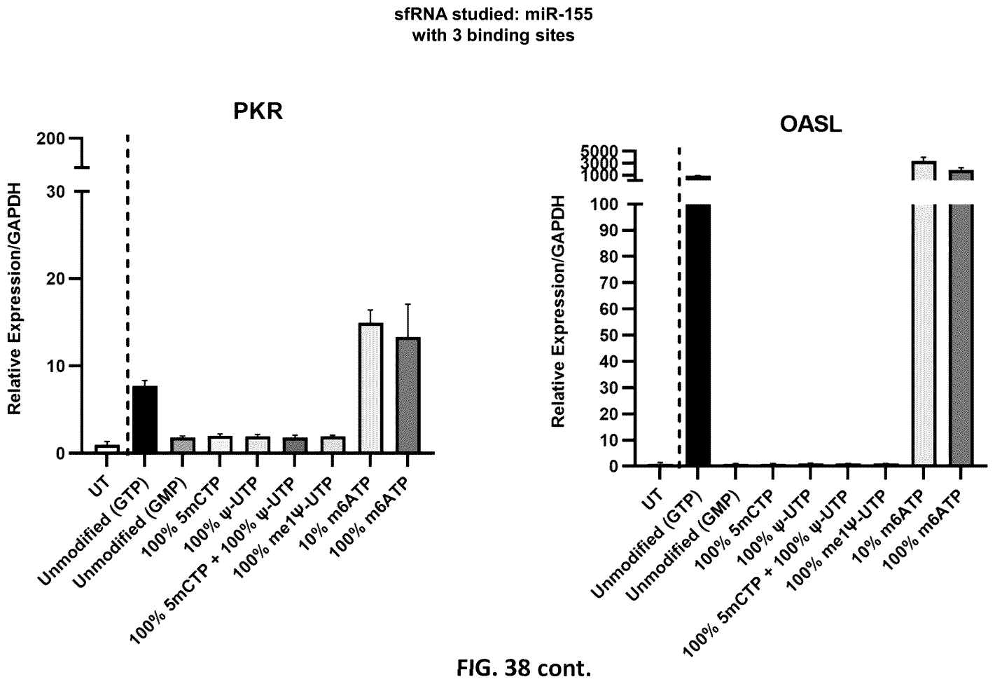

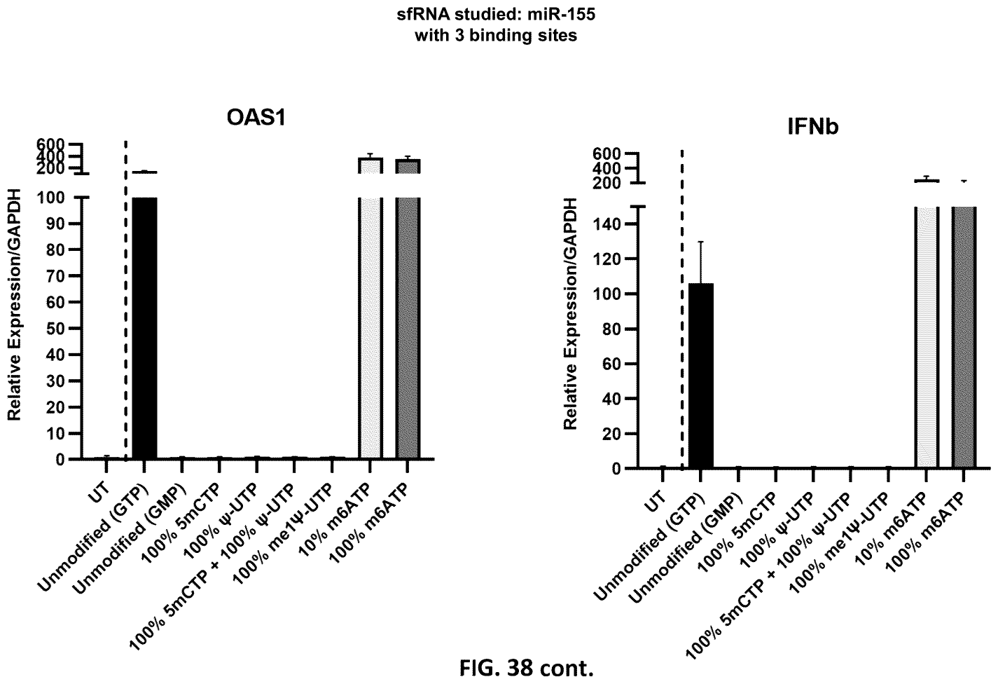

As shown in FIG. 38, unmodified GMP sfRNA showed complete reduction in the levels of RIG-I, MDA5, PKR, IFN-beta and OAS expression compared to unmodified GTP sfRNA. Together with observations from Example 15, converting the 5′ end from GTP to GMP for 5mCTP or Ψ-UTP modified sfRNAs resulted in a complete reduction in levels of innate immune genes (FIG. 37 and FIG. 38). As seen with 5mCTP and Ψ-UTP or me1Ψ-UTP modified GTP sfRNAs in Example 15, 5mCTP and Ψ-UTP or me1Ψ-UTP modified GMP sfRNAs showed reduced immunogenicity compared to unmodified GTP sfRNA (FIG. 38). 10% or 100% m6A modified sfRNAs failed to reduce immunogenicity regardless of 5′ GTP or 5′ GMP ends (FIG. 37 and FIG. 38).

Thus, the type of modification introduced together with the level of 5′ end phosphorylation influences activation of innate immune regulators.

### Example 17: sfRNA Targeting miR-132 with Modified Nucleotides Reduced Immunogenicity

A sfRNA targeting miR-132 engineered with complete or partial incorporation of modified nucleotides was produced. For modification substitution, the modified nucleotides, methylcytosine (5mCTP), pseudouridine (Ψ-UTP), N1-methylpseudouridine (me1Ψ-UTP) and m6A, were added in place of the standard unmodified nucleotides, cytosine, uridine and adenosine, respectively, during the in vitro transcription reaction.

Expression levels of innate immune response genes were measured in HEK293T cells transfected with unmodified or modified sfRNAs with either 5mCTP, Ψ-UTP, 5mCTP and Ψ-UTP, me1Ψ-UTP or m6A modifications. Total RNA was extracted using Trizol Reagent (Invitrogen) and subjected to reverse transcription to generate cDNA. qRT-PCR analysis for immune related genes was performed using SYBR green (Quantabio).

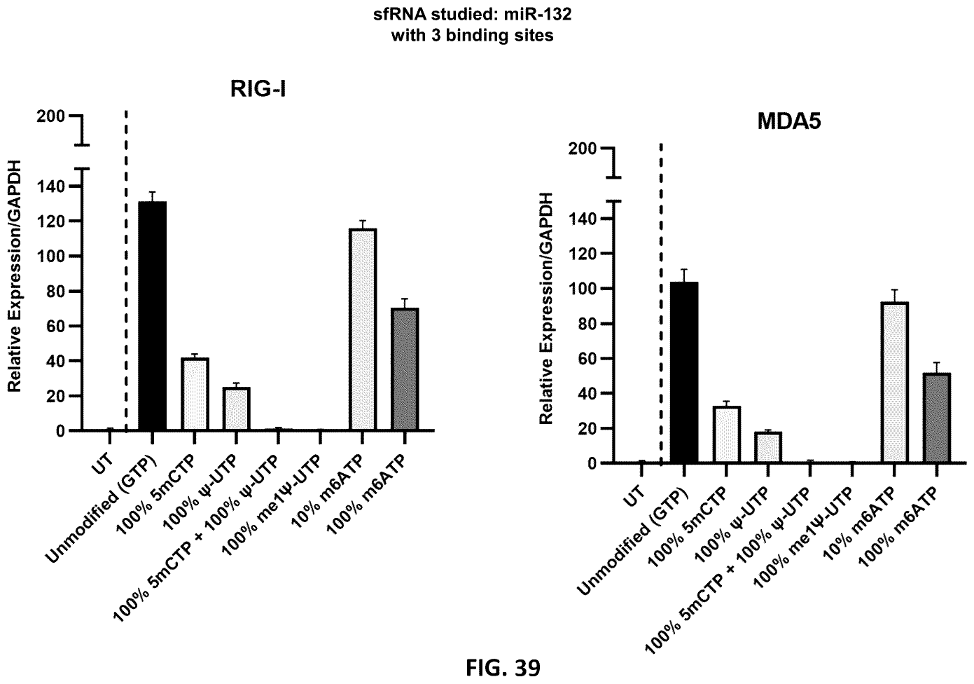

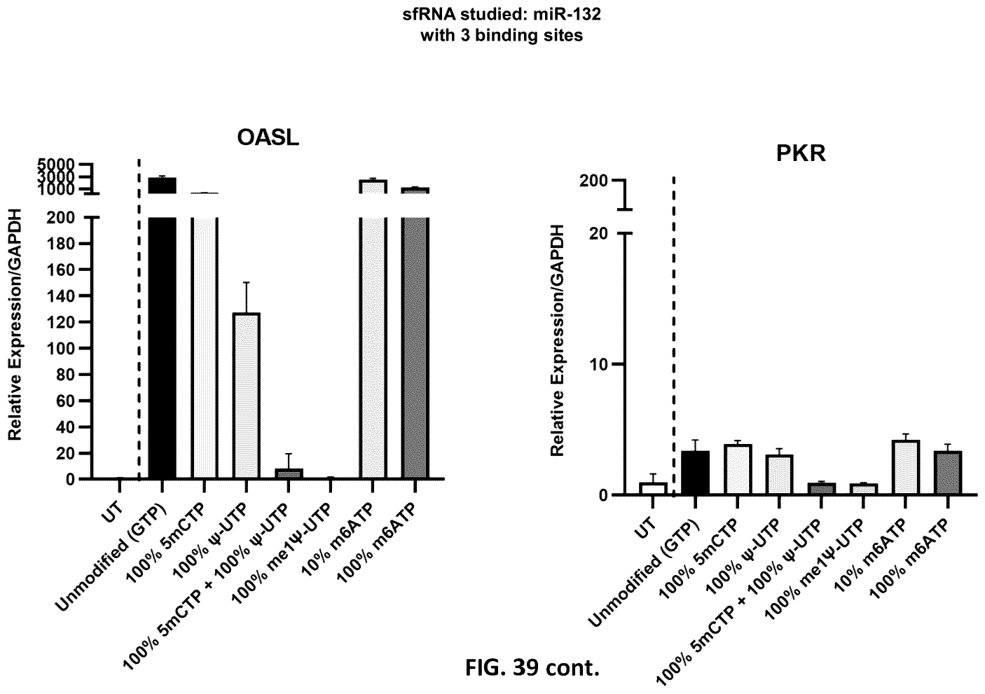

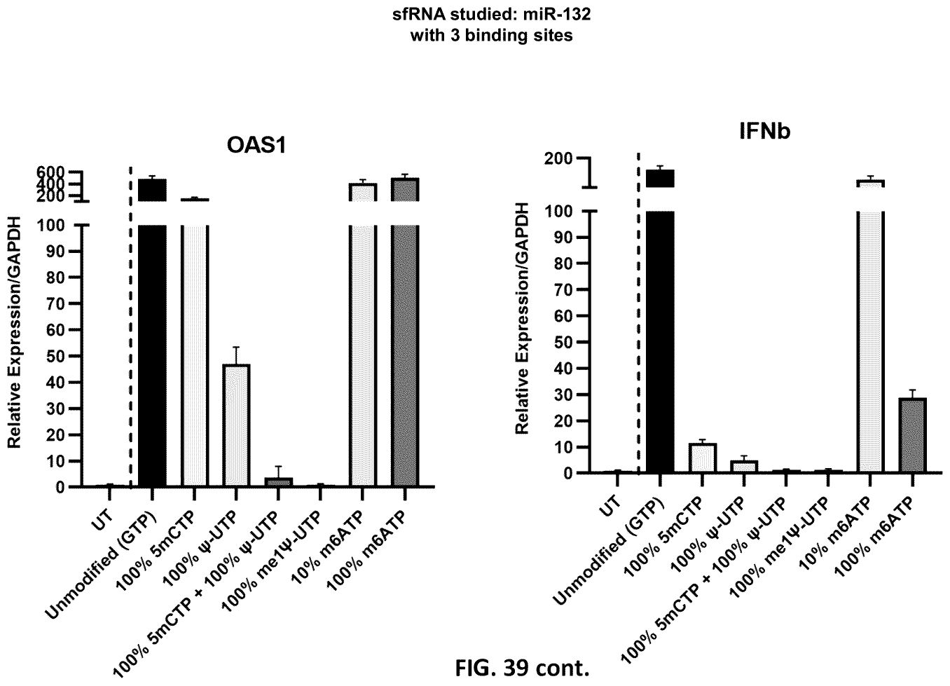

As shown in FIG. 39, 5mCTP and Ψ-UTP or me1Ψ-UTP modified sfRNAs showed complete reduction in the expression levels of RIG-I, MDA5, PKR. IFN-beta and OAS as compared to unmodified sfRNA. 5mCTP or Ψ-UTP modified sfRNA decreased levels of RIG-I. MDA5 and IFN-beta expression as compared to unmodified sfRNA (FIG. 39). m6A modified sfRNA failed to reduce immunogenicity except in instances where 100% m6A modified sfRNA decreased RIG-I. MDA5 and IFN-beta expression levels, although to a lesser extent than 5mCTP or Ψ-UTP modified sfRNA (FIG. 39).

Comparing modification substitutions in miR-155 sfRNA (FIG. 37) and miR-132 sfRNA (FIG. 39). 5mCTP or Ψ-UTP modifications reduced immunogenicity to a greater extent in miR-132 sfRNA than in miR-155 sfRNA. 5mCTP and Ψ-UTP or me1Ψ-UTP modifications in both miR-155 and miR-132 sfRNA eliminated immunogenicity (FIG. 37 and FIG. 39). Thus, the type of modification introduced and its influence on activation of innate immune regulators could vary across different sfRNA structures.

### Example 18: sfRNA Targeting miR-132 with Modified Nucleotides and 5′ Monophosphate End Reduced Immunogenicity

A sfRNA targeting miR-132 engineered with a 5′ monophosphate end and modified nucleotides was produced. sfRNAs produced via in vitro transcription carry 5′ triphosphate (GTP) ends by convention. To synthesise sfRNAs with 5′ monophosphate (GMP) ends. GMP was added 10-fold in excess to GTP during the in vitro transcription reaction. For modification substitution, the modified nucleotides, methylcytosine (5mCTP), pseudouridine (Ψ-UTP), N1-methylpseudouridine (me1Ψ-UTP) and m6A, were added in place of the standard unmodified nucleotides, cytosine, uridine and adenosine, respectively, during the in vitro transcription reaction.

Expression levels of innate immune response genes were measured in HEK293T cells transfected with unmodified sfRNAs with 5′ GTP or 5′ GMP ends or modified sfRNAs with 5′ GMP ends carrying either 5mCTP, Ψ-UTP. 5mCTP and Ψ-UTP, me1Ψ-UTP or m6A modifications. Total RNA was extracted using Trizol Reagent (Invitrogen) and subjected to reverse transcription to generate cDNA. qRT-PCR analysis for immune related genes was performed using SYBR green (Quantabio).

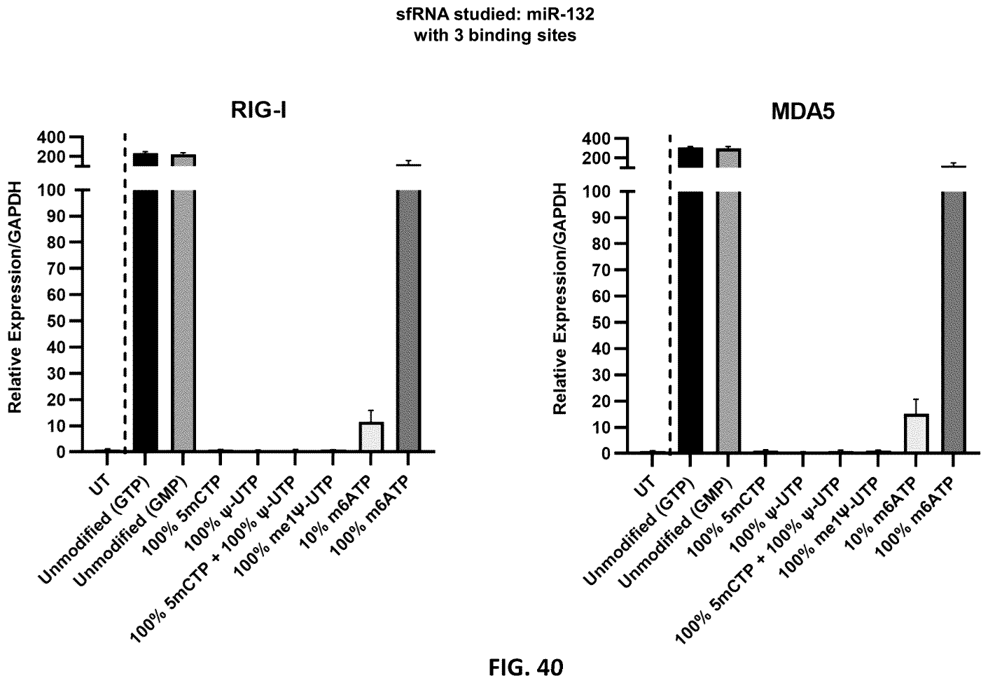

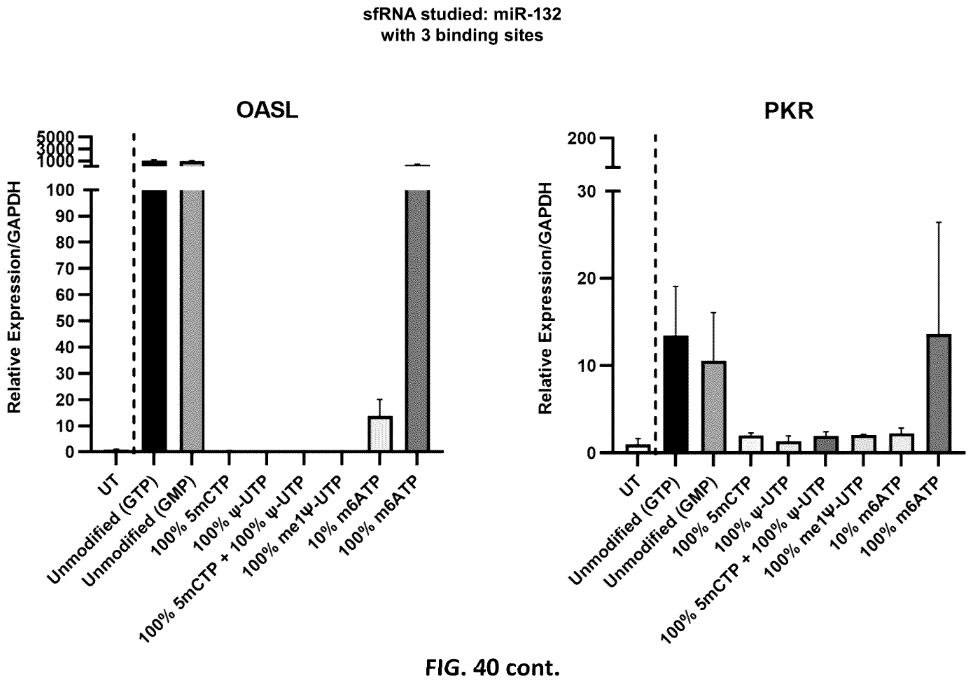

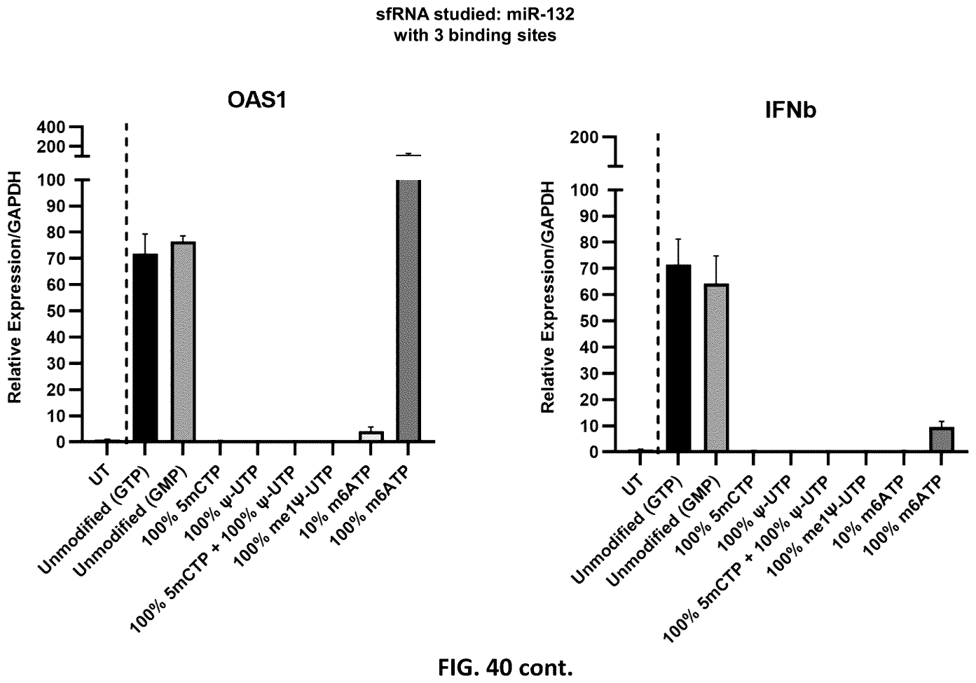

As shown in FIG. 40, unmodified GMP sfRNA showed a similar effect to unmodified GTP sfRNA, failing to reduce immunogenicity. Together with observations from Example 17, converting the 5′ end from GTP to GMP for 5mCTP or Ψ-UTP modified sfRNAs resulted in a complete reduction in levels of innate immune genes (FIG. 39 and FIG. 40). Similar to 5mCTP and Ψ-UTP or me1Ψ-UTP modified GTP sfRNAs in Example 17, 5mCTP and Ψ-UTP or me1Ψ-UTP modified GMP sfRNAs eliminated immunogenicity (FIG. 40). Notably, converting the 5′ end of 10% m6A modified sfRNA from GTP to GMP decreased expression levels of innate immune regulators (FIG. 40). 100% m6A modified sfRNAs failed to reduce immunogenicity regardless of 5′ GTP or 5′ GMP ends (FIG. 39 and FIG. 40).

Converting the 5′ end of 10% m6A modified sfRNA from GTP to GMP reduced immunogenicity for miR-132 sfRNAs but not miR-155 sfRNAs (FIG. 38 and FIG. 40). Thus, the type of modification introduced in combination with the level of 5′ end phosphorylation and its influence on activation of innate immune regulators could vary across different sfRNA structures

### Example 19: Determining the Structure of an Engineered Nucleic Acid

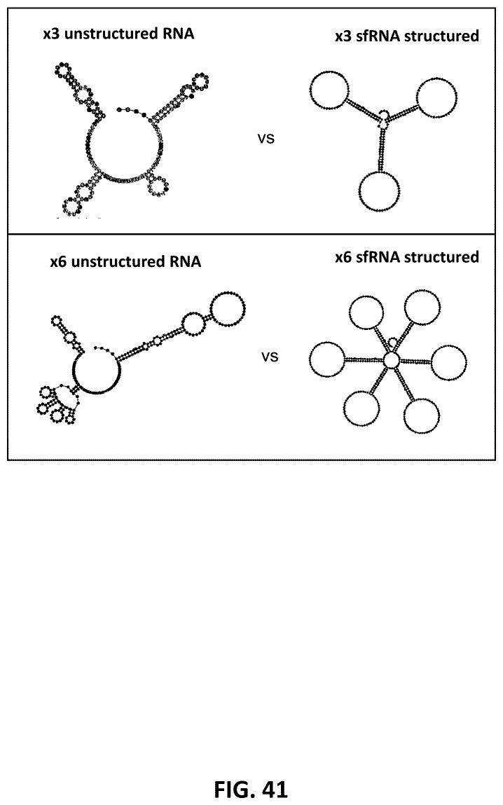

Engineered RNAs were tested as structured sfRNAs or unstructured RNA molecules with 3 and 6 binding sites as shown in FIG. 41. The engineered RNAs were tested for their ability to bind and sponge miR-155 mimics. Briefly, the functional efficacy of the structured sfRNA vs unstructured RNA was validated in a dual-luciferase reporter system assay described above.

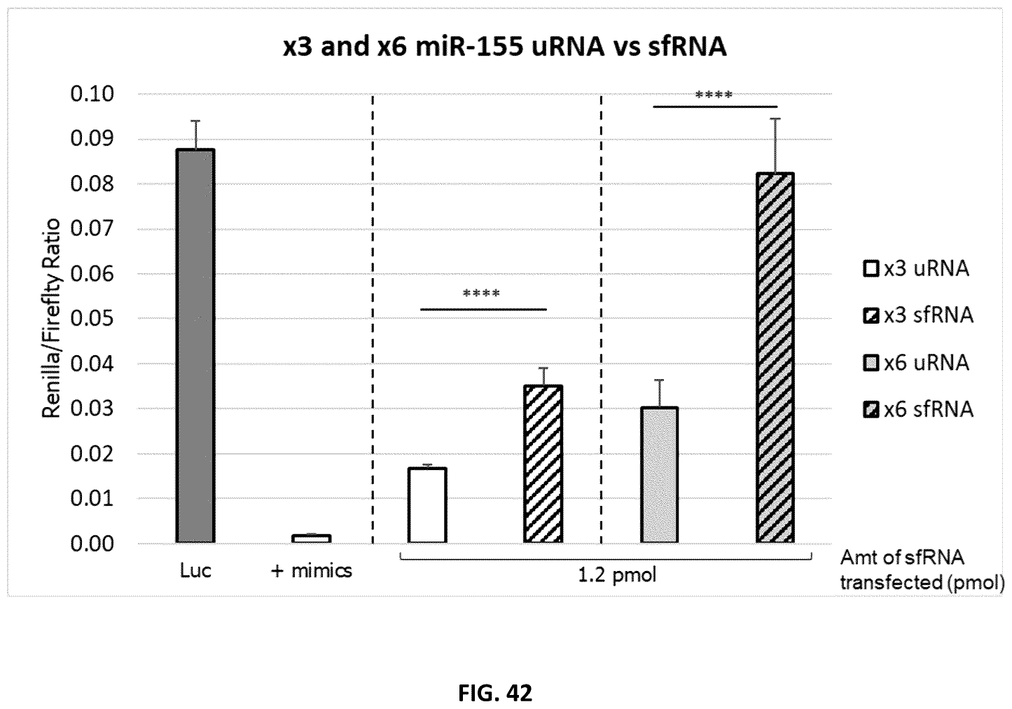

HEK293T cells were transfected with 25 ng luciferase vector. 0.5 pmol of miR-155 mimics and 1.2 pmol of in vitro T7 synthesized miR-155 structured sfRNA or in vitro T7 synthesized miR-155 unstructured RNA using Lipofectamine 2000 reagent. The luciferase assay was completed 48 hours post transfection. FIG. 42 shows the luciferase results for the 3× and 6× sfRNA vs the 3× and 6× unstructured RNA (uRNA). FIG. 42 showed in both 3 and 6 binding site constructs, sfRNA showed a greater rescue effect compared to unstructured RNA. Additionally, the rescue effect of sfRNA with 6 sites is greater than 3 sites, consistent with the previous Example 5 in which equimolar comparison of sfRNAs carrying an increasing number of binding sites (from 1-6) showed a proportional increase in rescue effect.

### Example 20: Determining Specificity of sfRNA Targeting miR-155 with 12 nt Arm No Deletion Stem Design

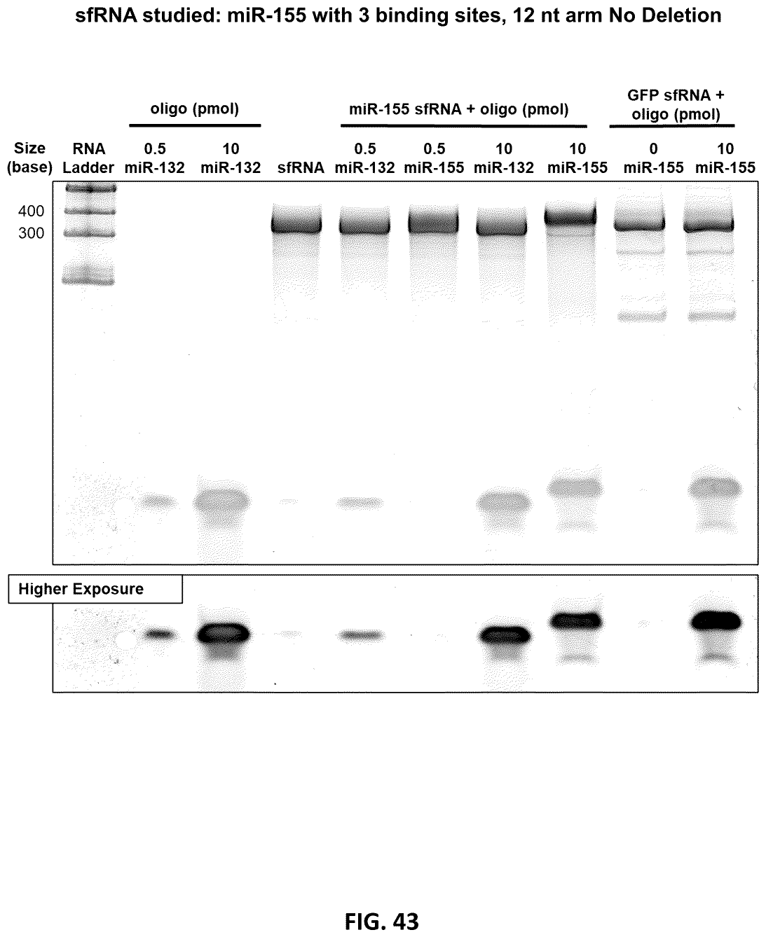

Engineered RNAs were tested for their specificity to sponge miR-155 as sfRNAs with 3 binding sites designed to bind to miR-155 with a 12 nt homology arm size containing no deletions. Briefly, increasing amounts of miRNA oligo (single stranded miR-155-5p or miR-132-3p) were added to a constant amount. 500 ng, of sfRNA. sfRNA configured to target GFP was used as a negative control. All samples were pre-incubated together in a low salt solution (10 mM Tris/100 mM NaCl) at 85° C. for 5 minutes and allowed to slow cool to room temperature. Samples were then heated to 95° C. in denaturing loading buffer and loaded onto a 10% TBE-Urea gel. The results are shown in FIG. 43.

FIG. 43 shows the engineered sfRNA bound to miR-155 but not to the miR-132 oligo. 0.5 pmol amounts of only miR-155 oligo, and not miR-132 oligo, showed complete hybridisation with sfRNA. Additionally, there was a clear-shift in the band representing the miR-155-sfRNA complex which was observed with 10 pmol amounts of miR-155 oligo but not with the miR-132 oligo. Overall, the miR-155 targeting sfRNA binds specifically to the intended miR-155 sequence it was designed to target.

### Example 21: Determining Specificity of sfRNA Targeting miR-155 with 15 nt Arm Base Deletion Stem Design

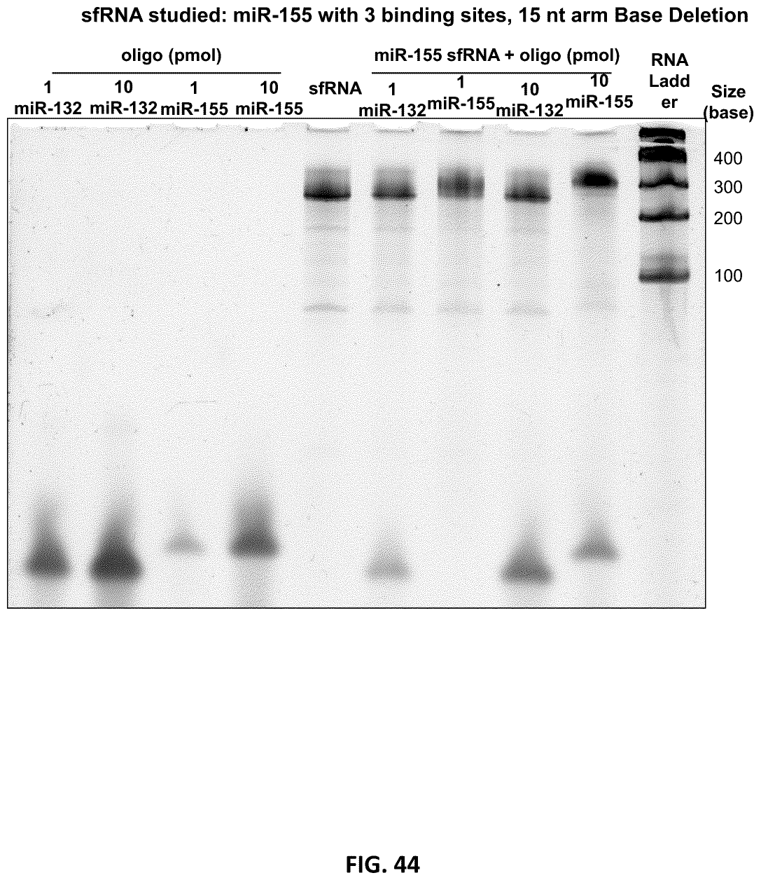

Engineered RNAs were tested for their specificity to sponge miR-155 as sfRNAs with 3 binding sites designed to bind to miR-155 with a 15 nt homology arm size containing a single base deletion in one strand of an homology arm. Briefly, increasing amounts of miRNA oligo (single stranded miR-155-5p or miR-132-3p) were added to a constant amount. 500 ng, of sfRNA. All samples were pre-incubated together in a low salt solution (10 mM Tris/100 mM NaCl) at 85° C. for 5 minutes and allowed to slow cool to room temperature. Samples were then heated to 95° C. in denaturing loading buffer and loaded onto a 10% TBE-Urea gel. The results are shown in FIG. 44.

FIG. 44 shows the engineered sfRNA bound to miR-155 but not to the miR-132 oligo. 1 pmol amounts of only miR-155 oligo, and not miR-132 oligo, showed complete hybridisation with sfRNA. Additionally, there was a clear-shift in the band representing the miR-155-sfRNA complex which was observed with 10 pmol amounts of miR-155 oligo but not with the miR-132 oligo. Overall, the miR-155 targeting sfRNA binds specifically to the intended miR-155 sequence it was designed to target regardless of the size or type of homology arm (i.e., 12 nt no deletion seen in FIG. 43).

### Example 22: Determining Specificity of sfRNA Targeting miR-132 with 15 nt Arm Base Deletion Stem Design

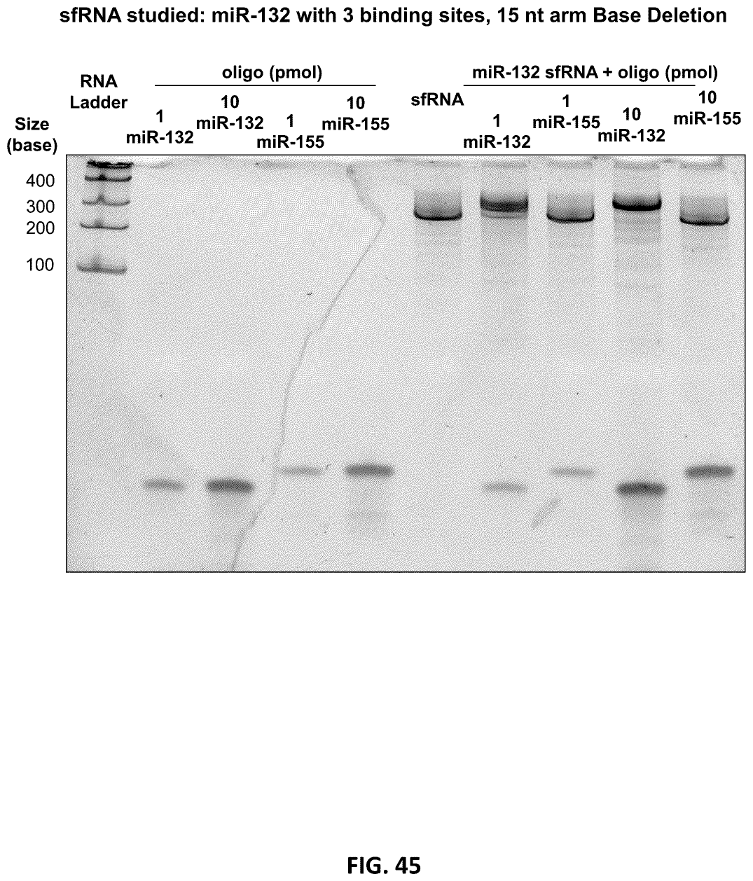

Engineered RNAs were tested for their specificity to sponge miR-132 as sfRNAs with 3 binding sites designed to bind to miR-132 with a 15 nt homology arm size containing a single base deletion in one strand of an homology arm. Briefly, increasing amounts of miRNA oligo (single stranded miR-155-5p or miR-132-3p) were added to a constant amount. 500 ng, of sfRNA. All samples were pre-incubated together in a low salt solution (10 mM Tris/100 mM NaCl) at 85° C. for 5 minutes and allowed to slow cool to room temperature. Samples were then heated to 95° C. in denaturing loading buffer and loaded onto a 10% TBE-Urea gel. The results are shown in FIG. 45.

FIG. 45 shows the engineered sfRNA bound to miR-132 but not to the miR-155 oligo. Additionally, there was a clear-shift in the band representing the miR-132-sfRNA complex which was observed with both 1 and 10 pmol amounts of miR-132 oligo but not with the miR-155 oligo. Notably, miR-132 targeting sfRNA was not able to show complete hybridisation of 1 pmol amounts of miR-132 oligo unlike miR-155 targeting sfRNA in FIG. 44. Overall, the miR-132 targeting sfRNA binds specifically to the intended miR-312 sequence it was designed to target.

### Example 23: sfRNA Targeting miR-155 with Modified Nucleotides Show Differences in Functional Effect

Engineered RNAs were tested with complete or partial incorporation of modified nucleotides as described above. The engineered RNAs were tested for their ability to bind miR-155 mimics. Briefly, the functional efficacy of the auxiliary arm lengths in an engineered polynucleotide was validated in a dual-luciferase reporter system assay described above.

HEK293T cells were transfected with 25 ng luciferase vector, 0.5 pmol of miR-155 mimics and 100 ng of in vitro T7 synthesized sfRNAs with different modifications using Lipofectamine 2000 reagent. The luciferase assay was completed 48 hours post transfection.

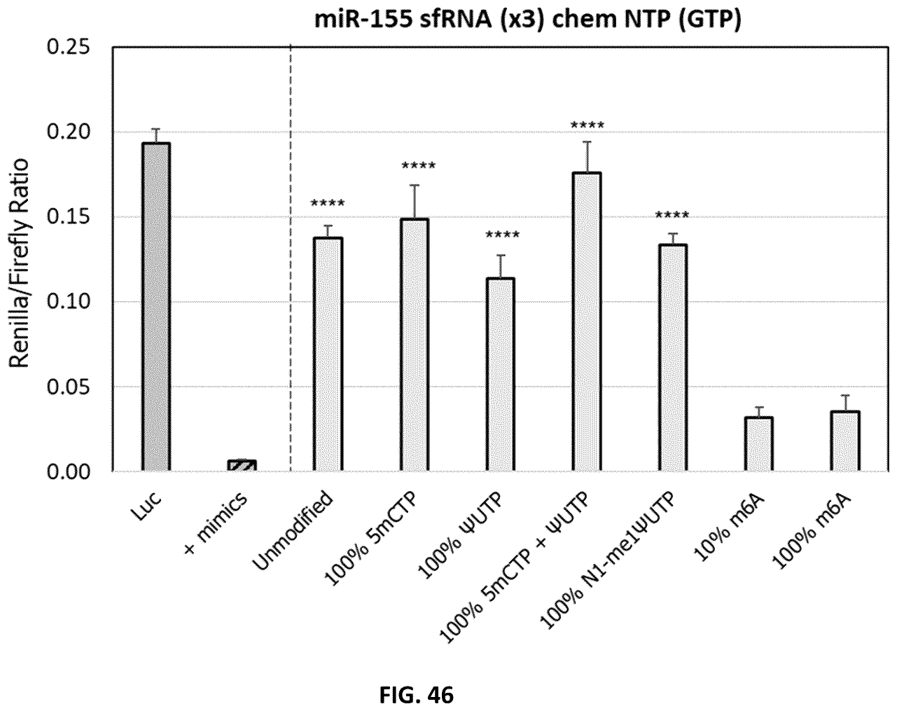

FIG. 46 shows the luciferase results for unmodified and modified miR-155 sfRNA with 5′ triphosphate end for 3 binding sites. Luciferase rescue effect was similar across unmodified and all modifications except m6A modifications as shown in FIG. 46. 10% or 100% m6A modified sfRNA failed to show a rescue effect (FIG. 46).

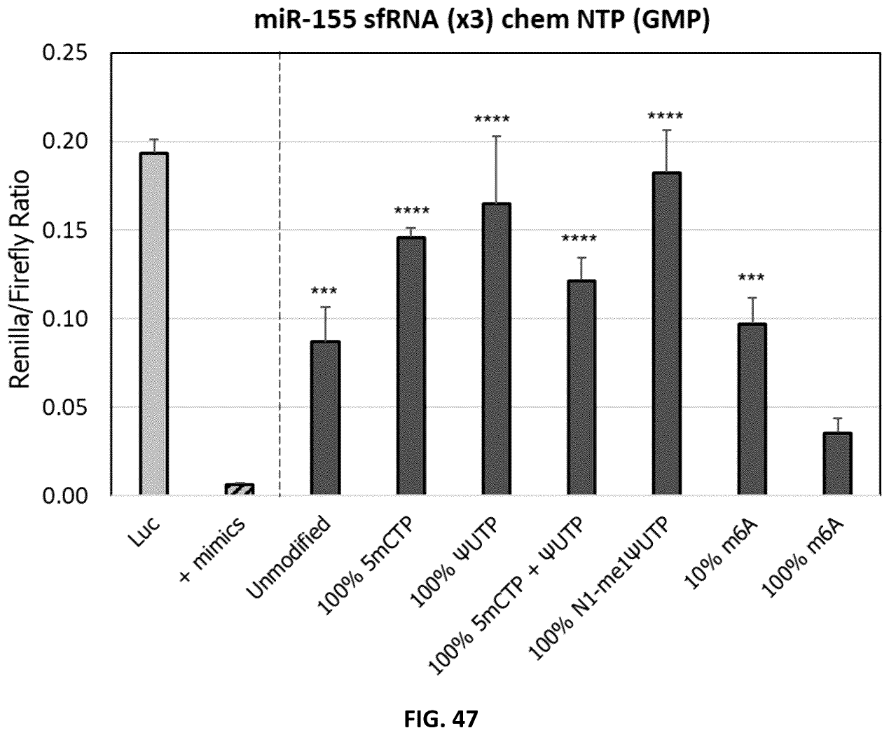

FIG. 47 shows the luciferase results for unmodified and modified miR-155 sfRNA with 5′ monophosphate end for 3 binding sites. Luciferase rescue effect was observed across unmodified and all modifications except for the 100% m6A modification as shown in FIG. 47. Thus, the modification introduced in sfRNA influences function.

### Example 24: sfRNA Targeting miR-132 with Modified Nucleotides Show Differences in Functional Effect

Engineered RNAs were tested with complete or partial incorporation of modified nucleotides as described above. The engineered RNAs were tested for their ability to bind miR-132 mimics. Briefly, the functional efficacy of the auxiliary arm lengths in an engineered polynucleotide was validated in a dual-luciferase reporter system assay described above.

HEK293T cells were transfected with 25 ng luciferase vector, 0.5 pmol of miR-132 mimics and 100 ng of in vitro T7 synthesized sfRNAs with different modifications using Lipofectamine 2000 reagent. The luciferase assay was completed 48 hours post transfection.

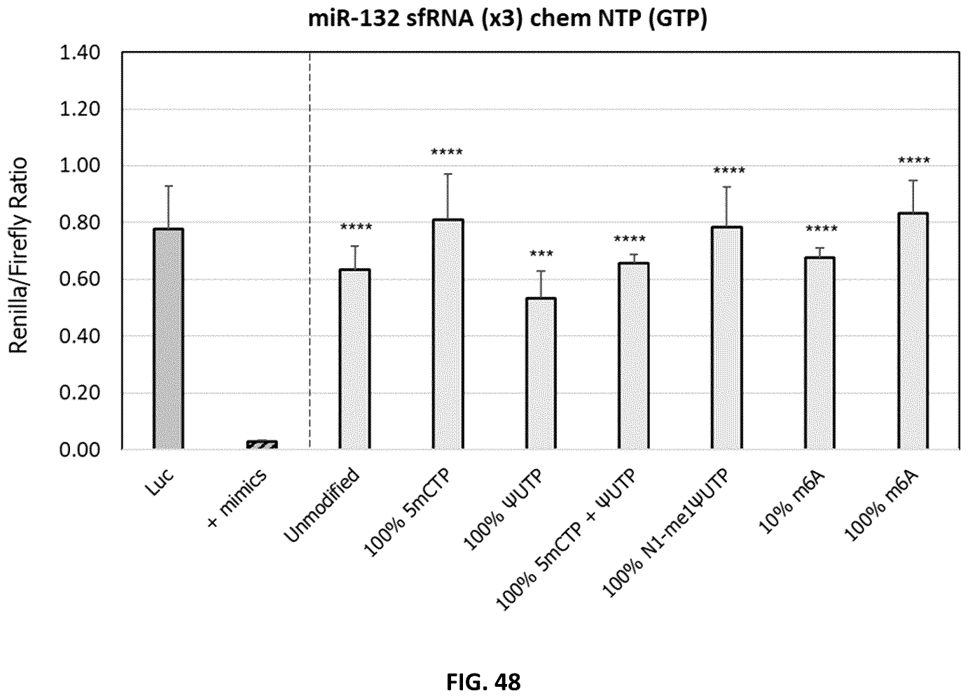

FIG. 48 shows the luciferase results for unmodified and modified miR-132 sfRNA with 5′ triphosphate end for 3 binding sites. Luciferase rescue effect was similar across unmodified and all modifications sfRNAs as shown in FIG. 48. Notably, 10% and 100% m6A modified miR-132 sfRNAs showed functional rescue of luciferase effect (FIG. 48) unlike m6A modified miR-155 sfRNAs (FIG. 46).

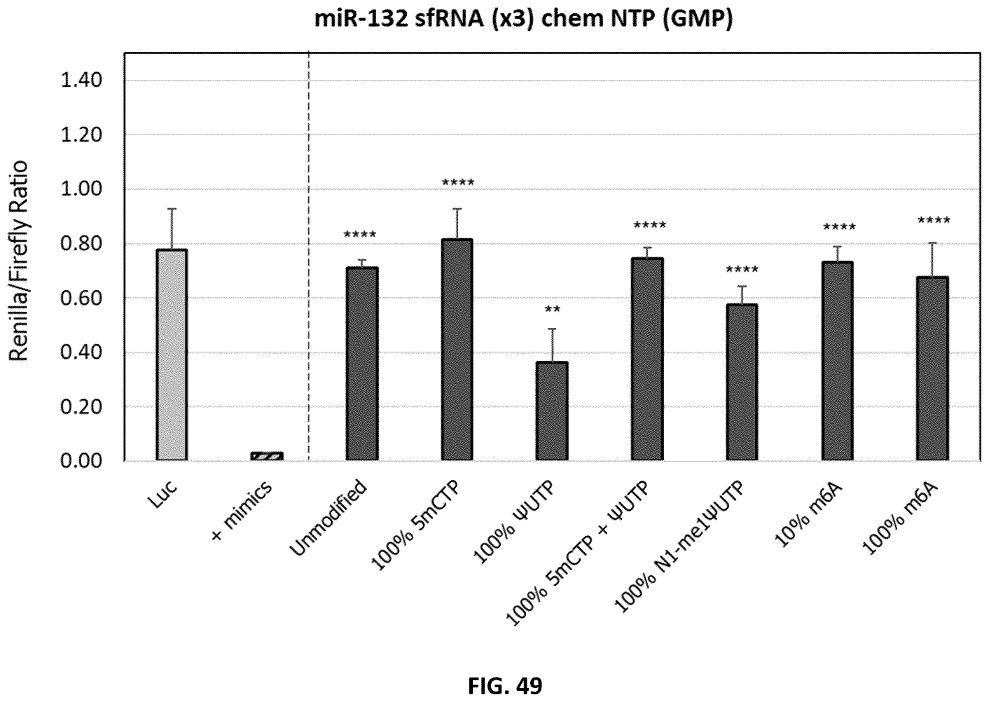

FIG. 49 shows the luciferase results for unmodified and modified miR-132 sfRNA with 5′ monophosphate end for 3 binding sites. Luciferase rescue effect was observed across unmodified and all modifications as shown in FIG. 49. Across all modifications, 100% Ψ-UTP modified sfRNA showed the lowest rescue effect. However, 100% m6A modified miR-132 sfRNA with 5′ monophosphate showed functional rescue (FIG. 49) unlike that observed with miR-155 sfRNA with 5′ monophosphate (FIG. 47). Thus, modified NTPs introduced in sfRNA exert different functional effects depending on the associated structure and sequence of the sfRNA targeting either miR-155 or miR-132.

While preferred aspects of the present disclosure have been shown and described herein, such aspects are provided by way of example only. Numerous variations, changes, and substitutions can occur. It should be understood that various alternatives to the aspects of the disclosure described herein may be employed in practicing the disclosure. It is intended that the following claims define the scope of the disclosure and that methods and structures within the scope of these claims and their equivalents be covered thereby

1. An engineered nucleic acid comprising a plurality of stem loops wherein each stem loop of the plurality is joined to at least one additional stem loop of the plurality of stem loops by a linker sequence, wherein each loop of each stem loop of the plurality of stem loops independently comprises a binding site that is independently configured to hybridize to a target RNA and when hybridized to the target RNA comprises a first mismatched base with respect to a first corresponding base of the target RNA, and wherein the engineered nucleic is not circularized.

2. The engineered nucleic acid of claim 1, which comprises from about 50 to about 550 nucleotides, and wherein the plurality of stem loops comprises 3, 4, 5, 6, 7, or 8 stem loops.

3. (canceled)

4. The engineered nucleic acid of claim 1, wherein each stem loop of the plurality of stem loops independently comprises a stem comprising from about 5 to about 20 base pairs or about 12 to about 15 base pairs.

5. The engineered nucleic acid of claim 4, wherein the stem comprises a deletion in one arm of the stem, a mismatch in the stem, or both.

6. (canceled)

7. The engineered nucleic acid of claim 1, wherein each linker sequence is independently from about 2 to about 12 nucleotides in length or about 6 to 8 nucleotides in length.

8. The engineered nucleic acid of claim 1, wherein each loop of each stem loop of the plurality of stem loops independently comprises from about 15 to about 75 nucleotides.

9. (canceled)

10. The engineered nucleic acid of claim 1, wherein each binding site of each loop of the plurality of stem loops, when independently hybridized to an individual target RNA, individually comprises a second mismatched base with respect to a second corresponding base of the target RNA.

11. The engineered of claim 1, wherein the first corresponding base of the target RNA is positioned within a first 8 nucleotides of a 5′ end of the target RNA.

12. The engineered nucleic acid of claim 1, wherein each stem loop of the plurality of stem loops is the same.

13. The engineered nucleic acid of claim 1, wherein at least one stem loop of the plurality of stem loops differs from the remaining stem loops of the plurality of stem loops.

14. The engineered nucleic acid of claim 1, wherein a binding site of a loop of a stem loop of the engineered nucleic acid is configured upon the binding or hybridizing to a target RNA to produce at least one mismatch between a base of the binding site of a loop of a stem loop and a base of the target RNA.

15.-23. (canceled)

24. The engineered nucleic acid of any one of claim 1, wherein the loop of a stem loop can comprise a first binding site and a second binding site.

25.-26. (canceled)

27. The engineered nucleic acid of claim 1, wherein each loop of each stem loop of the plurality of stem loops is configured to hybridize to an identical target RNA.

28. (canceled)

29. The engineered nucleic acid of any one of claim 1, wherein each stem loop of the plurality of stem loops is substantially radially disposed at a substantially regular distance around a partial loop comprising linkers.

30.-31. (canceled)

32. The engineered nucleic acid of claim 1, wherein the engineered nucleic acid comprises a modified nucleotide and wherein the modified nucleotide comprises pseudouridine, 5-methylcytidine, n1-methylpseudouridine, N6-methyladenosine, or any combination thereof.

33. (canceled)

34. The engineered nucleic acid of claim 1, further comprising a chemical modification and wherein the chemical modification comprises a phosphodiester modification, methylation of a hydroxyl group, an epigenetically marked base, a deoxyribose sugar, or a combination thereof.

35.-39. (canceled)

40. The engineered nucleic acid of any one of claim 1, wherein the target RNA is a messenger RNA (mRNA), a ribosomal RNA (rRNA), a transfer RNA (tRNA), a ribozyme a transfer messenger RNA (tmRNA), a double stranded RNA (dsRNA), a small nuclear RNA (ssRNA), a small nucleolar RNA (snoRNA), a Piwi-interacting RNAs (piRNA), a microRNA (miRNA), a small interfering RNA (siRNA), a long non-coding RNA (lncRNA), or any combination thereof.

41.-43. (canceled)

44. The engineered nucleic acid of claim 1, wherein the engineered nucleic acid comprises at least about 80%, 85%, 90%, 95%, 96%, 97%, 98%, or 99% sequence identity to any one of SEQ ID NOs: 253-330, 47-78, 331-426, 427-432, 433-511, or 512-583.

45.-52. (canceled)

53. A DNA encoding the engineered nucleic acid of claim 1.

54. A vector containing the engineered nucleic acid of claim 1.

55.-67. (canceled)

68. A pharmaceutical composition in unit dose form comprising the engineered nucleic acid of claim 1 and a pharmaceutically acceptable: excipient, diluent, or carrier.

69.-71. (canceled)

72. A kit comprising: the engineered nucleic acid of claim 1 and a container.

73. A method of treating or preventing a disease or condition in a subject, the method comprising administering to the subject a therapeutically effective amount of the pharmaceutical composition of claims 68.

74.-103. (canceled)


SEQUENCE LISTING (597 sequences)

SEQ ID NO: 1 - DNA, 120 bp, Human immunodeficiency virus 1
  source 1..120: mol_type = genomic DNA
ctttgagccaattcccatacattattgtgccccggctggttttgcgattctaaaatgtaataataagacgttcaatggaacaggaccatgtacaaatgtcagcacagtacaatgtacaca

SEQ ID NO: 2 - RNA, 20 nt, synthetic construct
  source 1..20: mol_type = other RNA
ttattacattttagaatcgc

SEQ ID NO: 3 - DNA, 744 bp, Dengue virus 3
  source 1..744: mol_type = genomic DNA
aatgaaatgggattgttggaaaccacaaagagagatttggggatgtccaaagaaccaggtgttgtttcgccaaccagttatttggatgtggatttacacccagcatcagcctggacattgtacgctgtggccacaacagtaataacaccaatgttgagacataccatagagaattccacagcaaatgtgtccctagcagctatagccaaccaggcagtggtcctgatgggtttagacaaaggatggccgatatcgaaaatggacttaggcgtgccactattggcactgggttgctattcacaagtgaacccactaactctcacagcggcagtactcctgctagtcacgcattatgctattataggtccaggattgcaggcaaaagctactcgtgaggctcaaaaaaggacggctgctggaataatgaagaatccaacggtggatgggataatgacaatagacctagatcctgtaatatacgactcaaaatttgaaaaacaactaggacaagttatgctcctggttctgtgtgcagttcaacttttgttaatgagaacatcatgggcattttgtgaagctctaaccctagccacaggaccaataacaacactctgggaaggatcacctgggaagttctggaacaccacgatagctgtttccatggcaaacatctttagagggagctatctggcaggagctgggctagctttttctatcatgaaatcagttggaacaggaaagaga

SEQ ID NO: 4 - RNA, 20 nt, synthetic construct
  source 1..20: mol_type = other RNA
agtgccaatagtggcacgcc

SEQ ID NO: 5 - DNA, 2764 bp, Severe acute respiratory syndrome coronavirus 2
  source 1..2764: mol_type = genomic DNA
tcagctgatgcacaatcgtttttaaacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacag

SEQ ID NO: 6 - RNA, 20 nt, synthetic construct
  source 1..20: mol_type = other RNA
ctagccactagaccttgaga

SEQ ID NO: 7 - DNA, 1215 bp, Epstein-Barr virus
  source 1..1215: mol_type = genomic DNA
atggaaaccactcagactctccgctttaagaccaaggccctagccgtcctgtccaagtgctatgaccatgcccagactcatctcaagggaggagtgctgcaggtaaaccttctgtctgtaaactatggaggcccccggctggccgccgtggccaacgcaggcacggccgggctaatcagcttcgaggtctcccctgacgctgtggccgagtggcagaatcaccagagcccagaggaggccccggccgccgtgtcatttagaaaccttgcctacgggcgcacctgtgtcctgggcaaggagctgtttggctcggctgtggagcaggcttccctgcaattttacaagcggccacaagggggttcccggcctgaatttgttaagctcactatggaatatgatgataaggtgtccaagagccaccacacctgcgccctgatgccctatatgcccccggccagcgacaggctgaggaacgagcagatgattgggcaggtgctgttgatgcccaagacggcttcctcgttgcagaagtgggcacgccagcaaggctcaggcggcgttaaggtgacactcaatccggatctctacgtcaccacgtatacttctggggaggcctgcctcaccctagactacaagcctctgagtgtggggccatacgaggccttcactggccctgtggccaaggctcaggacgtgggggccgttgaggcccacgttgtctgctcggtagcagcggactcgctggcggcggcgcttagcctctgccgcattccggccgttagcgtgccaatcttgaggttttacaggtctggcatcatagctgtggtggccggcctgctgacgtcagcgggggacctgccgttggatcttagtgttattttatttaaccacgcctccgaagaggcggccgccagtacggcctctgagccagaagataaaagtccccgggtgcaaccactgggcacaggactccaacaacgccccagacatacggtcagtccatctccttcacctccgccacctcctaggacccctacttgggagagtccggcaaggccagagacaccctcgcctgccattcccagccactccagcaacaccgcactggagaggcctctggctgttcagctcgcgaggaaaaggacatcgtcggaggccaggcagaagcagaagcaccccaagaaagtgaagcaggcctttaaccccctcatttaa

SEQ ID NO: 8 - RNA, 20 nt, synthetic construct
  source 1..20: mol_type = other RNA
cagtgaaggcctcgtatggc

SEQ ID NO: 9 - DNA, 1016 bp, Influenza A virus
  source 1..1016: mol_type = genomic DNA
ggagcaaaagcaggtagatattgaaagatgagtcttctaaccgaggtcgaaacgtacgttctctctatcatcccgtcaggccccctcaaagccgagatcgcacagagacttgaagatgtatttgctggaaagaataccgatcttgaggctctcatggagtggctaaagacaagaccaatcctgtcacctctgactaaggggattttaggatttgtgttcacgctcaccgtgcccagtgagcgaggactgcagcgtagacgctttgtccaaaatgccctcaatgggaatggggatccaaataatatggacagagcagtcaaactttatcgaaagcttaagagggagataacattccatggggccaaagaaatagcactcagttattctgctggtgcacttgccagttgtatgggactcatatacaacaggatgggggctgtgaccaccgaatcagcatttggccttatatgtgcaacctgtgaacagattgccgactcccagcataagtctcacaggcaaatggtaacaacaaccaatccattaataagacatgagaacagaatggttctggccagcactacagctaaggctatggagcaaatggctggatcgagcgaacaagcagctgaggccatggaggttgctagtcaggccaggcagatggtgcaggcaatgagagccattgggactcatcctagctctagcactggtctgaaaaatgatctccttgaaaatttacaggcctatcagaaacgaatgggggtgcagatgcaacgattcaagtgatcctcttgttgttgccgcaagtataattgggattgtgcacttgatattgtggattattgatcgccttttttccaaaagcatttatcgtatctttaaacacggtttaaaaagagggccttctacggaaggagtaccagagtctatgagggaagaatatcgagaggaacagcagaatgctgtggatgctgacgatgatcattttgtcagcatagagctagagtaaaaaactc

SEQ ID NO: 10 - RNA, 20 nt, synthetic construct
  source 1..20: mol_type = other RNA
tgcagtcctcgctcactggg

SEQ ID NO: 11 - DNA, 6384 bp, Lyssavirus rabies
  source 1..6384: mol_type = genomic DNA
atgctcgatcctggagaggtctatgatgaccctattgacccaatcgagttagaggctgaacccagaggaacccccattgtccccaacatcttgaggaactctgactacaatctcaactctcctttgatagaagatcctgctagactaatgttagaatggttaaaaacagggaatagaccttatcggatgactctaacagacaattgctccaggtctttcagagttttgaaagattatttcaagaaggtagatttgggttctctcaaggtgggcggaatggctgcacagtcaatgatttctctctggttatatggtgcccactctgaatccaacaggagccggagatgtataacagacttggcccatttctattccaagtcgtcccccatagagaagctgttgaatctcacgctaggaaatagagggctgagaatccccccagagggagtgttaagttgccttgagagggttgattatgataatgcatttggaaggtatcttgccaacacgtattcctcttacttgttcttccatgtaatcaccttatacatgaacgccctagactgggatgaagaaaagaccatcctagcattatggaaagatttaacctcagtggacatcgggaaggacttggtaaagttcaaagaccaaatatggggactgctgatcgtgacaaaggactttgtttactcccaaagttccaattgtctttttgacagaaactacacacttatgctaaaagatcttttcttgtctcgcttcaactccttaatggtcttgctctctcccccagagccccgatactcagatgacttgatatctcaactatgccagctgtacattgctggggatcaagtcttgtctatgtgtggaaactccggctatgaagtcatcaaaatattggagccatatgtcgtgaatagtttagtccagagagcagaaaagtttaggcctctcattcattccttgggagactttcctgtatttataaaagacaaggtaagtcaacttgaagagacgttcggtccctgtgcaagaaggttctttagggctctggatcaattcgacaacatacatgacttggtttttgtgtttggctgttacaggcattgggggcacccatatatagattatcgaaagggtctgtcaaaactatatgatcaggttcaccttaaaaaaatgatagataagtcctaccaggagtgcttagcaagcgacctagccaggaggatccttagatggggttttgataagtactccaagtggtatctggattcaagattcctagcccgagaccaccccttgactccttatatcaaaacccaaacatggccacccaaacatattgtagacttggtgggggatacatggcacaagctcccgatcacgcagatctttgagattcctgaatcaatggatccgtcagaaatattggatgacaaatcacattctttcaccagaacgagactagcttcttggctgtcagaaaaccgaggggggcctgttcctagcgaaaaagttattatcacggccctgtctaagccgcctgtcaatccccgagagtttctgaggtctatagacctcggaggattgccagatgaagacttgataattggcctcaagccaaaggaacgggaattgaagattgaaggtcgattctttgctctaatgtcatggaatctaagattgtattttgtcatcactgaaaaactcttggccaactacatcttgccactttttgacgcgctgactatgacagacaacctgaacaaggtgtttaaaaagctgatcgacagggtcaccgggcaagggcttttggactattcaagggtcacatatgcatttcacctggactatgaaaagtggaacaaccatcaaagattagagtcaacagaggatgtattttctgtcctagatcaagtgtttggattgaagagagtgttttctagaacacacgagttttttcaaaaggcctggatctattattcagacagatcagacctcatcgggttacgggaggatcaaatatactgcttagatgcgtccaacggcccaacctgttggaatggccaggatggcgggctagaaggcttacggcagaagggctggagtctagtcagcttattgatgatagatagagaatctcaaatcaggaacacaagaaccaaaatactagctcaaggagacaaccaggttttatgtccgacatacatgttgtcgccagggctatctcaagaggggctcctctatgaattggagagaatatcaaggaatgcactttcgatatacagagccgtcgaggaaggggcatctaagctagggctgatcatcaagaaagaagagaccatgtgtagttatgacttcctcatctatggaaaaacccctttgtttagaggtaacatattggtgcctgagtccaaaagatgggccagagtctcttgcgtctctaatgaccaaatagtcaacctcgccaatataatgtcgacagtgtccaccaatgcgctaacagtggcacaacactctcaatctttgatcaaaccgatgagggattttctgctcatgtcagtacaggcagtctttcactacctgctatttagcccaatcttaaagggaagagtttacaagattctgagcgctgaaggggagagctttctcctagccatgtcaaggataatctatctagatccttctttgggagggatatctggaatgtccctcggaagattccatatacgacagttctcagaccctgtctctgaagggttatccttctggagagagatctggttaagctcccaagagtcctggattcacgcgttgtgtcaagaggctggaaacccagatcttggagagagaacactcgagagcttcactcgccttctagaagatccgaccaccttaaatatcagaggaggggccagtcctaccattctactcaaggatgcaatcagaaaggctttatatgacgaggtggacaaggtggaaaattcagagtttcgagaggcaatcctgttgtccaagacccatagagataattttatactcttcttaatatctgttgagcctctgtttcctcgatttctcagtgagctattcagttcgtcttttttgggaatccccgagtcaatcattggattgatacaaaactcccgaacgataagaaggcagtttagaaagagtctctcaaaaactttagaagaatccttctacaactcagagatccacgggattagtcggatgacccagacacctcagagggttgggggggtgtggccttgctcttcagagagggcagatctacttagggagatctcttggggaagaaaagtggtaggcacgacagttcctcacccttctgagatgttgggattacttcccaagtcctctatttcttgcacttgtggagcaacaggaggaggcaatcctagagtttctgtatcagtactcccgtcctttgatcagtcatttttttcacgaggccccctaaagggatacttgggctcgtccacctctatgtcgacccagctattccatgcatgggaaaaagtcactaatgttcatgtggtgaagagagctctatcgttaaaagaatctataaactggttcattactagagattccaacttggctcaagctctaattaggaacattatgtctctgacaggccctgatttccctctagaggaggcccctgtcttcaaaaggacggggtcagccttgcataggttcaagtctgccagatacagcgaaggagggtattcttctgtctgcccgaacctcctctctcatatttctgttagtacagacaccatgtctgatttgacccaagacgggaagaactacgatttcatgttccagccattgatgctttatgcacagacatggacatcagagctggtacagagagacacaaggctaagagactctacgtttcattggcacctccgatgcaacaggtgtgtgagacccattgacgacgtgaccctggagacctctcagatcttcgagtttccggatgtgtcgaaaagaatatccagaatggtttctggggctgtgcctcacttccagaggcttcccgatatccgtctgagaccaggagattttgaatctctaagcggtagagaaaagtctcaccatatcggatcagctcaggggctcttatactcaatcttagtggcaattcacgactcaggatacaatgatggaaccatcttccctgtcaacatatacggcaaggtttcccctagagactatttgagagggctcgcaaggggagtattgataggatcctcgatttgcttcttgacaagaatgacaaatatcaatattaatagacctcttgaattggtctcaggggtaatctcatatattctcctgaggctagataaccatccctccttgtacataatgctcagagaaccgtctcttagaggagagatattttctatccctcagaaaatccccgccgcttatccaaccactatgaaagaaggcaacagatcaatcttgtgttatctccaacatgtgctacgctatgagcgagagataatcacggcgtctccagagaatgactggctatggatcttttcagactttagaagtgccaaaatgacgtacctatccctcattacttaccagtctcatcttctactccagagggttgagagaaacctatctaagagtatgagagataacctgcgacaattgagttctttgatgaggcaggtgctgggcgggcacggagaagataccttagagtcagacgacaacattcaacgactgctaaaagactctttacgaaggacaagatgggtggatcaagaggtgcgccatgcagctagaaccatgactggagattacagccccaacaagaaggtgtcccgtaaggtaggatgttcagaatgggtctgctctgctcaacaggttgcagtctctacctcagcaaacccggcccctgtctcggagcttgacataagggccctctctaagaggttccagaaccctttgatctcgggcttgagagtggttcagtgggcaaccggtgctcattataagcttaagcctattctagatgatctcaatgttttcccatctctctgccttgtagttggggacgggtcaggggggatatcaagggcagtcctcaacatgtttccagatgccaagcttgtgttcaacagtcttttagaggtgaatgacctgatggcttccggaacacatccactgcctccttcagcaatcatgaggggaggaaatgatatcgtctccagagtgatagatcttgactcaatctgggaaaaaccgtccgacttgagaaacttggcaacctggaaatacttccagtcagtccaaaagcaggtcaacatgtcctatgacctcattatttgcgatgcagaagttactgacattgcatctatcaaccggatcaccctgttaatgtccgattttgcattgtctatagatggaccactctatttggtcttcaaaacttatgggactatgctagtaaatccaaactacaaggctattcaacacctgtcaagagcgttcccctcggtcacagggtttatcacccaagtaacttcgtctttttcatctgagctctacctccgattctccaaacgagggaagtttttcagagatgctgagtacttgacctcttccacccttcgagaaatgagccttgtgttattcaattgtagcagccccaagagtgagatgcagagagctcgttccttgaactatcaggatcttgtgagaggatttcctgaagaaatcatatcaaatccttacaatgagatgatcataactctgattgacagtgatgtagaatcttttctagtccacaagatggttgatgatcttgagttacagaggggaactctgtctaaagtggctatcattatagccatcatgatagttttctccaacagagtcttcaacgtttccaaacccctaactgacccctcgttctatccaccgtctgatcccaaaatcctgaggcacttcaacatatgttgcagtactatgatgtatctatctactgctttaggtgacgtccctagcttcgcaagacttcacgacctgtataacagacctataacttattacttcagaaagcaagtcattcgagggaacgtttatctatcttggagttggtccaacgacacctcagtgttcaaaagggtagcctgtaattctagcctgagtctgtcatctcactggatcaggttgatttacaagatagtgaagactaccagactcgttggcagcatcaaggatctatccagagaagtggaaagacaccttcataggtacaacaggtggatcaccctagaggatatcagatctagatcatccctactagactacagttgcctgtga

SEQ ID NO: 12 - RNA, 20 nt, synthetic construct
  source 1..20: mol_type = other RNA
tataatgagcaccggttgcc

SEQ ID NO: 13 - DNA, 1908 bp, Variola virus
  source 1..1908: mol_type = genomic DNA
ttataattttaccatttgactcatgacttcattgatatctttatacgagctacgaatatataattctttataactgaactgagatatatacactggatctatggtttccataattgagtaaataaatgctctgcaataactaatggcaaatgtataaaacaacaaaattatactagagttgttaaagttaatattttctattagttgttccaataaattatttgttgtgactgtgttcaagtcataaatcattttgatactatccaataaacagtctttaagttctggaatattatcatcccattgtaaagcccctaattcgactatcgaataccctgctctggtagccgtttcaatatcgacggacgtcaatactgtaataaaggtggtagtattgtcatcatcatgatacacaacaggaatatggtcgttagtaggtacggtgactttacacaacgcgatatataactttccttttgtgccatttttaacgtagttgggacgtcctgcagggtattgttttgaagaaatgatatcgagaacagatttgatacgatatttgttggatttctgattattcactataatataatctagacagatagatgattcgataaataaagaagatatatcgttggtaggataatacatccccattccagtattcttggatactctattgatgacactagttaagaacatgtcttcaattctagaaaacgaaaacatcctacacggactcattaaaacttctagcgctcctgattgtgtctcaaatgcctcatacaatgatttcaaggatgtcatagattctttaacccacgatttaatattgcgtttagcatctattttttttattaaatcgaatggtcggctctctggtttgctaccccaatgataacaatagtcttgtaaagataaaccgcaagaaaatttatacgcatccatccaaataactcttgcaccatcagatgatatcagtatattattatagattttccatccacaattattgggccagtatactgttagcaacggtatatcgaatagattactcatgtaacctactagaatgatagttcgtgtactagtcataatatctttaatccaatctaagaaatttaaaattagatcttttacactgttaaagttaacaaaggtattacccgggtacgtggatatcatatatggcattggtccattatcagtaatagctccataaactgatacggtgatggtttttatatgtgtttgatctaacgaggaagaaatttgcgcccataattcatctctagatatgtatttaatatcaaacggtaacacatcaatttcgggacgcgtatatgtttctaaatttttaatccaaatataatgatgacctatatgccctattatcatactgtcaactatagtacacttagagaacttacgatacatctgtttcctataatcgttaaattttacaaatctataacatgctaaaccttttgacgacaaccattcattaatttctgatatggaatctgtattctcgatacagtatcgttttaaagccagtgctatatctccctgttcgtgagaacgctttcgtataatatcaatcaacggataatctgaggtttttggagaataatatgactcatgatctatttcgtccataaacaatctagacataggaattggaggcgatgatcttaattttgtgcaatgagtagttaatcctataacttctaatcttgtaatattcatcatcgacataatactatctatgttatcatcgtatattagtataccatgaccttcttcatttcgtgccaaaatgatatacagtcttaaatagttacgcaatatctcaatagtttcataattgttagctgttttcattaagatttgtaccctgtttaacat

SEQ ID NO: 14 - RNA, 20 nt, synthetic construct
  source 1..20: mol_type = other RNA
cagggagatatagcactggc

SEQ ID NO: 15 - DNA, 365 bp, Homo sapiens
  source 1..365: mol_type = genomic DNA
gagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgctataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttga

SEQ ID NO: 16 - RNA, 20 nt, synthetic construct
  source 1..20: mol_type = other RNA
tatagcagcaggtgactggg

SEQ ID NO: 17 - DNA, 2923 bp, Homo sapiens
  source 1..2923: mol_type = genomic DNA
aggctgtgacagtcatctgtctggacgcgctgggtggatgcggggggctcctgggaactgtgttggagccgagcaagcgctagccaggcgcaagcgcgcacagactgtagccatccgaggacacccccgcccccccggcccacccggagacacccgcgcagaatcgcctccggatcccctgcagtcggcgggaggtaaggagcagggcttgcaaaccgcccggcgcccagggaagcgacgagcgccggggcaaggcaagccctggacgggattgcgacgtgcgcaccgggcgccctaatatgcccgggggactgtttctgcttccgaaacaaaaccatctctgggttttcccagaaaagccagttccagccccgaaggcatcctggctagaggagacccgccctaatccttttgcagcccttaccggggggagtaatggcttctgcgaaaagaaattccctcggctctagaagatctgtctgtgtttgagctgtcggagagccgtgttggaggtcggcgccggcccccgccttccgcgccccccacgggaaggaagcacccccggtattaaaacgaacggggcggaaagaagccctcagtcgccggccgggaggcgagccgatgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctagacgcttctcaaaactggacagtcactgccactttgcacattttgattttttttttaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttctttttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggaagttcatacctaagtactgtaataatacctcaatgtttgaggagcatgttttgtatacaaatatattgttaatctctgttatgtactgtactaattcttacactgcctgtatactttagtatgacgctgatacataactaaatttgatacttatattttcgtatgaaaatgagttgtgaaagttttgagtagatattactttatcactttttgaactaagaaacttttgtaaagaaatttactatatatatatgcctttttcctagcctgtttcttcctgttaatgtatttgttcatgtttggtgcatagaactgggtaaatgcaaagttctgtgtttaatttcttcaaaatgtatatatttagtgctgcatcttatagcactttgaaatacctcatgtttatgaaaataaatagcttaaaattaaa

SEQ ID NO: 18 - RNA, 20 nt, synthetic construct
  source 1..20: mol_type = other RNA
ccgaactcgacaggggaccg

SEQ ID NO: 19 - DNA, 2456 bp, Homo sapiens
  source 1..2456: mol_type = genomic DNA
gcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatcta

SEQ ID NO: 20 - RNA, 20 nt, synthetic construct
  source 1..20: mol_type = other RNA
ctctgccaggacgctcatcc

SEQ ID NO: 21 - DNA, 5250 bp, Homo sapiens
  source 1..5250: mol_type = genomic DNA
gccgtgctagccgttgggcctgcctcggaggaggcgtcgccgccgccgctgccgctgccggcgccgttgccgctgccgggaaacacaaggaaagggaaccagcgcagcgtggcgatgggcgggggtagagccccgccggagaggctgggcggctgccggtgacagactgtgccctgtccacggtgcctcctgcatgtcctgctgccctgagctgtcccgagctaggtgacagcgtaccacgctgccaccatgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcggaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgagtgagcagtctgcccacgcagaggacgcacgctcgctgccatcaccgctgggtggttttttccccctaactttttacttagcctttttggtttgtgtccccacccccacctcctcaccccctttccagttcttcttcaggcccctcccagacgcaccccagcggcccctgcagcccctgcctccagcctccagcctcacctttgtgcccagactcgcatttggaagactccacctcccgcccaggcctgggctgttgggcggttggagattcaggttttaatccacacaagccccagtgaggggtgaagcatggcgcctggggcctgcctgagtttctggcctgggtgtcgtgctggtgtctgcctccgcgctgctgcatctggacgaaggctgccttctggtgggacgcgacacccggcagacagtggtgctgccttccaggccccgtggcctaggctcggagtggccaggcacggggcggtccaatcccccacccgctgtccccctatgggggcagaaaagcaataatgtccaggggcaggcaggggcccttgggagctgcagggctgggggttagggctgctccctggtgaatggagtcagatcctaggatctgtaccatggggaaccaggagtggccgggctgggtgccgcctcctggtccggcctcctccccaccaaactgtcctcaccctatggatgaggcaggaggaacatttggggccaaacctgcctgcctcccagccccgtgccttactagggcttccttccagctggccttacctcccgctggaccctgggcctggcctggccccactgggggctatgggctgggctcaccctctcctctgcgggggtggagggccaccagccttggctgttacaatcttacaccggacagtattgggccccatggacttggtcagggaggggtgggggtgggcatctctggtacctattggggtggggggcctctgaaaagggaggctcctaggcccccctcacccctccctctccccagggccccacgttctgcagccttaaggttgaacatgagtgcacgtccatgtcagtgctgtgggactcctgtgcgtgcctcggactgcgtgtgtcggcgggacgcaggcacacgtgggtgtgtgtgcatgtgtgtttgtgtgagggcagcgtgtcctccagtgtgcatggtgtgtgggcttgggccccatccctggcccgagcatttcatcctgtgggggaggggtgctgacctagtgggaggagccccactgtgatccatgagctgccctgcccacgcctcccctccctgtagcaacacctctgggtgtttggagtttagcttttgtgggtttgctctccctatcccatctcctgtactacacagttcatggcagggtggggaggggtggggttggttcgggtgggtgagggtctttttcctctgtgtgcgatgttgttatctgacagttctccgtccctactggcctttctcctcgtcttcatatttgtacggtacaagcaataaagacactcatttcagaccagggcccagcctgcactcacgccagcccaaccactctgggctttgccttggtgatggagtcagacccctgggccccagctcctcctgtactagccgttcccttcagcaaggagggcactgagctcagggtgagggcagctggggtgtgtgcaggagctcaggctggagagggtgggtggagctggtgctgtggggctgaggggtatgggaaggctccccgcatgtgggggtggggtggacagagaccactccaggccctcagtgctgcttaggctaagagaggtggggtggagggacagggctggaagatctgggtagcccagaatgaggagggtgcctgtgctgtcactgaatgagagggagtggttcattccacccggctgccgagcctcagaggggggcattcctatcctgccccacctccctgtttatgctgccacctggaagccttgaggcccccaaattccagtacagacccagtggtgtgttcatggtggcgtggttgctgtcacctgggagctcctgagcgtttggttagaaccctgttcagcttggggtcagccctcccctagtcactgccctttagcctggatgtgtctgggcccctgcacttcccgtgcttgagtcacgtggctgcatggccgggcgctggccggatggaacacctcccccagcaagggaccagggaccagagccctggcctgccctgctgagccctgctgtgcagagggcctggcacagatgaatttgagattttgccgcaaggtgttagcacttcacacccattgagtctttgagattttaagtgaatgtaagcagaaaaagtcagatccaatttacagaaatcagagttagctacagctaggactcgtttggttggggttttttagtttgtctttctaaagtcatgtggaccttaatttaattacaaaagtctaccctggtggtcataaaataggcaggcctatgaagaaaggccttttactcttccatctcgtcccagccccgagttgacccacgttgctgctcctcacaccatggtgatgcaggtctcgtagtgtgggcacaggcctggctacctcatctttttagtgcctctctcctcttccacaggatggggtcccacagctgcagcagctggccccgtagttgagcatgtgtggttatcctgtagagcttttcccaagaagggtgtttgaacttagagtcttaataaaatcttaccaaataaattttgagtagaataatcgtcttttgcaatgtacattttaaaaatttcacacattcttttttgtatataaagaacagtgactgggcacagtggctcatgcctgtaatcccagcaatttgggaggccgaggcgggcgggtctcttgaggccaggggttcgagaccagcctgggcatcatagggagaccttcatctctacaaaaaatacaaaaattagctgggcatggtggtgcatgcctgcaatcccagctaacttggaaggctgaggtgaggtgggaagatcacttgagcccaggagtttgaggctgcagtgagctatgattgcggcactgcactgcagcctgggacaatgagactgtgtctctaaaaataaaaaaaaaaaaaacatgatacatgctattaaaaaagacagcaaagcaggagtataagaaaggaaattcacccgaggtcgcagggccttgagtactcattttggtgctgattacctctctgcaaatggacacggcatcataaattggtagtttcctgctctttttgtgtaatcttttccagttaatgtgaagcctctgggggctgccctcgtgcactgatggttgtgtggagtcgggggcggcagtgcgattcccttttagctgctgcatggggggaactcaggctttccagctgcttcctggggttccatggggtagacccctcaaccgcttcagctgccccgttaacaggaattgacttggtttcgtttggtgctaccagcagtcctgtaataaactagctatccatctgta

SEQ ID NO: 22 - RNA, 20 nt, Homo sapiens
  source 1..20: mol_type = genomic RNA
agctcctgctcacaccccag

SEQ ID NO: 23 - DNA, 779 bp, Shigella sonnei
  source 1..779: mol_type = genomic DNA
cccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggtgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgctgccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgatag

SEQ ID NO: 24 - RNA, 20 nt, Shigella sonnei
  source 1..20: mol_type = genomic RNA
atggcttcattcagctccgg

SEQ ID NO: 25 - DNA, 595 bp, Klebsiella pneumoniae
  source 1..595: mol_type = genomic DNA
atgatagaaatggatctggccagcggccgcacgctgaccgcctggcgcgccgatgaacgctttcccatgatgagcacctttaaagtagtgctctgcggcgcagtgctggcgcgggtggatgccggtgacgaacagctggagcgaaagatccactatcgccagcaggatctggtggactactcgccggtcagcgaaaaacatcttgccgacggcatgacggtcggcgaactctgtgccgccgccattaccatgagcgataacagcgccgccaatctgctgctggccaccgtcggcggccccgcaggattgactgcctttttgcgccagatcgacgacaacgtcacccgccttgaccgctgggaaacggaactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgatcgggtcgccggaccgttgatccgctccgtgctgccggcgggctggtttatcgccgataagaccggagctggcgagcggggtgcgcgcg

SEQ ID NO: 26 - RNA, 20 nt, Klebsiella pneumoniae
  source 1..20: mol_type = genomic RNA
ccggcatccacccgcgccag

SEQ ID NO: 27 - DNA, 876 bp, Escherichia coli
  source 1..876: mol_type = genomic DNA
atggtgacaaagagagtgcaacggatgatgttcgcggcggcggcgtgcattccgctgctgctgggcagcgcgccgctttatgcgcagacgagtgcggtgcagcaaaagctggcggcgctggagaaaagcagcggagggcggctgggcgtcgcgctcatcgataccgcagataatacgcaggtgctttatcgcggtgatgaacgctttccaatgtgcagtaccagtaaagttatggcggccgcggcggtgcttaagcagagtgaaacgcaaaagcagctgcttaatcagcctgtcgagatcaagcctgccgatctggttaactacagtccgattgccgaaaaacacgtcaacggcacaatgacgctggcagaactgagcgcggccgtgttgcagtacagcgacaataccgccatgaacaaattgattgcccagctcggtggcccgggaggcgtgacggcttttgcccgcgcgatcggcgatgagacgtttcgtctggatcgcactgaacctacgctgaataccgccattcccggcgacccgagagacaccaccacgccgcgggcgatggcgcagacgttgcgtcagcttacgctgggtcatgcgctgggcgaaacccagcgggcgcagttggtgacgtggctcaaaggcaatacgaccggcgcagccagcattcgggccggcttaccgacgtcgtggactgtgggtgataagaccggcagcggcggctacggcaccaccaatgatattgcggtgatctggccgcagggtcgtgcgccgctggttctggtgacctattttacccagccgcaacagaacgcagagagccgccgcgatgtgctggcttcagcggcgagaatcatcgccgaagggctgtaa

SEQ ID NO: 28 - RNA, 20 nt, Escherichia coli
  source 1..20: mol_type = genomic RNA
acgtctgcgccatcgcccgc

SEQ ID NO: 29 - DNA, 612 bp, Proteus mirabilis
  source 1..612: mol_type = genomic DNA
aggaactgaaggttgttttttactttacgatgcatccacaaacgctgaaattgctcaattcaataaagcaaagtgtgcaacgcaaatggcaccagattcaactttcaagatcgcattatcacttatggcatttgatgcggaaataatagatcagaaaaccatattcaaatgggataaaacccccaaaggaatggagatctggaacagcaatcatacaccaaagacgtggatgcaattttctgttgtttgggtttcgcaagaaataacccaaaaaattggattaaataaaatcaagaattatctcaaagattttgattatggaaatcaagacttctctggagataaagaaagaaacaacggattaacagaagcatggctcgaaagtagcttaaaaatttcaccagaagaacaaattcaattcctgcgtaaaattattaatcacaatctcccagttaaaaactcagccatagaaaacaccatagagaacatgtatctacaagatctggataatagtacaaaactgtatgggaaaactggtgcaggattcacagcaaatagaaccttacaaaacggatggtttgaagggtttattataagcaaatcaggacataa

SEQ ID NO: 30 - RNA, 20 nt, Proteus mirabilis
  source 1..20: mol_type = genomic RNA
gattaataattttacgcagg

SEQ ID NO: 31 - DNA, 659 bp, Raoultella ornithinolytica
  source 1..659: mol_type = genomic DNA
attaaacaaagcgaaagccagctgtcgggccgcgtaggcatgatagaaatggatctggccagcggccgcacgctgaccgcctggcgcgccgatgaacgctttcccatgatgagcacctttaaagtagtgctctgcggcgcagtgctggcgcgggtggatgccggtgacgaacagctggacgaaagatccactatcgccagcaggatctggtggactactcgccggtcagcgaaaaacaccttgccgacggcatgacggtcggcgaactctgcgccgccgccattaccatgagcgataacagcgccgccaatctgctgctggccaccgtcggcggccccgcaggattgactgcctttttgcgccagatcggcgacaacgtcacccgccttgaccgctgggaaacggaactgaatgaggcgcttcccggcgacgcccgcgacaccactaccccggccagcatggccgcgaccctgcgcaagctgctgaccagccagcgtctgagcgccgttcgcaacggcagctgctgcagtggatggtggacgatcgggtcgccggaccgttgatccgctccgtgctgccgaacggcgctcagacgctggggcgggctggtttatcgccgataagaccggagctagcagcggggtgcgcgcgggattgtc

SEQ ID NO: 32 - RNA, 20 nt, Raoultella ornithinolytica
  source 1..20: mol_type = genomic RNA
gaacggcgctcagacgctgg

SEQ ID NO: 33 - DNA, 741 bp, Klebsiella oxytoca
  source 1..741: mol_type = genomic DNA
atgagcaagttatttgtattctttatgtttttgttttgtagcattactgccgcagcagagtctttgccagatttaaaaattgagaggcttgatgaaggcgtttatgttcatacttcgtttgaagaagttaacggttggggtgttgttcctaaacacggcttggtggttcttgtaaatactgaggcctatctgattgacactccatttacggctaaagatactgaaaagttagtcacttggtttgtgggacgcggctataaaataaaaggcagtatttcctctcattttcatagcgacagcacgggcggaatagagtggcttaattctcaatctatccccacgtatgcatctgaattaacaaatgaacttcttaaaaaagacggtaaggtacaagctaaaaattcatttggcggagttagctattggctagttaagaataagattgaagttttttatcctggtccagggcacactccagataacgtagtggtttggctacctgaaaatagagttttgttcggtggttgttttgttaaaccgtacggtcttggtaatttgggtgacgcaaatttagaagcttggccaaagtccgccaaattattaatgtccaaatatggtaaggcaaaactggttgttccaagtcacagtgaagttggagacgcatcactcttgaagcgaacattagaacatgcggttaaagggttaaatgaaagtaaaaaaccatcaaaaccaagtaactaa

SEQ ID NO: 34 - RNA, 20 nt, Klebsiella oxytoca
  source 1..20: mol_type = genomic RNA
caagtcacagtgaagttgga

SEQ ID NO: 35 - DNA, 914 bp, Enterobacter hormaechei
  source 1..914: mol_type = genomic DNA
gttatgccgcacccacccctatggagtcttgatgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcacgcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtagcagatgcggcataacaaatcgttggagcgggacttttgctacgcaggctgcgcctactccgcaaaagcccctcaactcaggc

SEQ ID NO: 36 - RNA, 20 nt, Enterobacter hormaechei
  source 1..20: mol_type = genomic RNA
gaaggacatcaacgccgccg

SEQ ID NO: 37 - DNA, 789 bp, Pseudomonas aeruginosa
  source 1..789: mol_type = genomic DNA
atgcgccctctcctcttgagtgcccttctcctgctttccgggcatacccaggccagcgaatggaacgacagccaggccgtggacaagctattcggcgcggccggggtgaaaggcaccttcgtcctctacgatgtgcagcggcagcgctatgtcggccatgaccgggagcgcgcggaaacccgcttcgttcccgcttccacctacaaggtggcgaacagcctgatcggcttatccacaggggcggttagatccgccgacgaggttcttccctatggcggcaagccccagcgcttcaaggcctgggagcacgacatgagcctgcgcgaggcgatcaaggcatcgaacgtaccggtctaccaggaactggcgcggcgcatcggcctggagcggatgcgcgccaatgtctcgcgcctgggttacggcaacgcggaaatcggccaggttgtggataacttctggttggtgggaccgctgaagatcagcgcgatggaacagacccactttctgctccgactggcgcagggagaattgccattccccgccccggtgcagtccaccgtgcgcgccatgaccctgctggaaagcggcccgggctgggagctgcacggcaagaccggctggtgcttcgactgcacgccggaactcggctggtgggtgggctgggtgaagcgcaacgagcggctctacggcttcgccctgaacatcgacatgcccggcggcgaggccgacatcggcaagcgcgtcgaactgggcaaggccagtctcaaggctctcgggatactgccctga

SEQ ID NO: 38 - RNA, 20 nt, Pseudomonas aeruginosa
  source 1..20: mol_type = genomic RNA
ccagcagggtcatggcgcgc

SEQ ID NO: 39 - DNA, 234 bp, Pseudomonas aeruginosa
  source 1..234: mol_type = genomic DNA
gccgcatatcggcgacggcctgaagccggtgcagcgacgcatcgtctacgccatgagcgaactggggctggatgccgattccaagcacaagaagtcggcgcgcaccgtcggcgacgtgctcggcaagttccacccgcacggcgattcggcctgctacgaggccatggtgctgatggcgcagccgttctcctatcgctatccgctggtggacggccagggcaactggggggctcc

SEQ ID NO: 40 - RNA, 20 nt, Pseudomonas aeruginosa
  source 1..20: mol_type = genomic RNA
catggcgtagacgatgcgtc

SEQ ID NO: 41-44 - skipped (4 entries)

SEQ ID NO: 45 - DNA, 1710 bp, Candida albicans
  source 1..1710: mol_type = genomic DNA
ttttggagataaccactagcgaagtaagacatattacaaaaatttccagccactattttgtacctgtaactttcaaaaatgattacactttttgacttcgttatcagtggcttcatattatatagtacttatcaaagctttaatacttttgaggtatcataaccttaagaaattgttttcaatctattactcgtctcacacggttagaagtcatttgtaggattttcagcagaacaaccatttttgttgtatagaagatagacgccaagaaaaaaacaagagtacgttatactttgaacaagatacacaaagagttaaataccgtaaaaacgcaaacttcattcttgactgcaaaagcgcctaaacttgtaaagcccgcccacaaatcttacatacaggagatattgagtaatcactacaaaccctataatcgtcaacaattcaaaacgaattgcacccaataggatcagtaaattctgctcaaaatatgtaaaagatacttttgcaattgctttattttattatttgttactttttttagtcgtacttgtcatgctatttttaagtggcttctcataaatgaagggctcagaattgaagatataaatacatcaaataacccacctatttcaaaattgttttatttttgtttatctctacaacaaacaactttcctttattttactaccaactattttccattcgttaaaatgaaattctccaaaattgcctgtgctactgtttttgctttatcttctcaagctgccatcatccaccatgctccagaattcaacatgaagagagatgttgctccagctgccccagctgctccagctgaccaagcacctactgttcctgcacctcaagaattcaatactgctattaccaaaagaagtattattggaattattatgggtattcttggcaacattccacaagtaatccaaatcatcatgagtattgtcaaagctttcaaaggtaacaagagagaagatattgattctgttgttgctggtatcattgctgatatgccatttgttgtcagagctgttgacacagccatgacttctgttgcttctaccaagagagatggagctaatgatgacgttgctaatgccgtcgtcagattgccagaaattgttgctcgtgttgccactggtgttcaacaatccatcgaaaatgccaagagagatggcgttccagatgttggccttaatcttgttgctaatgctccaagacttatctctaacgtttttgatggcgtcctggaaactgttcaacaagctaagagagatggtcttgaagattttcttgatgaacttcttcaaagactcccacaactcattactagatcagctgaatctgctttgaaagacagtcaaccagttaaaagagatgccggctcagtagcacttagcaatttaatcaaaaagagcattgaaactgtcggtattgaaaatgctgctcaaattgtttcagaaagagatatttcttctttgattgaagaatatttcggaaaagcttaaatgctcagcagataaaaatttgttttccacaagcttaatcttttattccataagtcttgtgacacttttgctgtaactgatttttttaattgttgttttttgtggtggtgttgaattgctttaatatcattttgatagcattcacaccatagtttatattacctatattttacttagatatcaa

SEQ ID NO: 46 - DNA, 20 bp, Candida albicans
  source 1..20: mol_type = genomic DNA
ttgatatctaagtaaaatat

SEQ ID NO: 47 - RNA, 76 nt, synthetic construct
  source 1..76: mol_type = other RNA
tgatatacgaccagtgacgactaagcaacccctatcacgtaagcattaagaaactcaagatactggtcgtatatca

SEQ ID NO: 48 - RNA, 75 nt, synthetic construct
  source 1..75: mol_type = other RNA
tgatatacgaccagtgcgcactacacaacccctatcacgtaagcattaagaaactcaagatactggtcgtatatc

SEQ ID NO: 49 - RNA, 79 nt, synthetic construct
  source 1..79: mol_type = other RNA
tgatatacgaccagtgcgcactacacaacccctatcacgtaagcattaagaaactcaagatactggtcgtatatcattt

SEQ ID NO: 50 - RNA, 79 nt, synthetic construct
  source 1..79: mol_type = other RNA
tgataagaaatgacagcgcactacacaacccctatcacgtaagcattaagaaactcaagattgtcatttcttatcattt

SEQ ID NO: 51 - RNA, 79 nt, synthetic construct
  source 1..79: mol_type = other RNA
cattagtatgttaaggcgcactacacaacccctatcacgtaagcattaagaaactcaagatcttaacatactaatgttt

SEQ ID NO: 52 - RNA, 79 nt, synthetic construct
  source 1..79: mol_type = other RNA
tcaatacagattattgcgcactacacaacccctatcacgtaagcattaagaaactcaagataataatctgtattgattt

SEQ ID NO: 53 - RNA, 79 nt, synthetic construct
  source 1..79: mol_type = other RNA
ttatatacttcaagggcgcactacacaacccctatcacgtaagcattaagaaactcaagatccttgaagtatataattt

SEQ ID NO: 54 - RNA, 79 nt, synthetic construct
  source 1..79: mol_type = other RNA
ttgtttattgacattgcgcactacacaacccctatcacgtaagcattaagaaactcaagataatgtcaataaacaattt

SEQ ID NO: 55 - RNA, 79 nt, synthetic construct; identical to SEQ ID NO: 49
  source 1..79: mol_type = other RNA

SEQ ID NO: 56 - RNA, 79 nt, synthetic construct
  source 1..79: mol_type = other RNA
tgataagaaatgactgcgcactacacaacccctatcacgtaagcattaagaaactcaagatagtcatttcttatcattt

SEQ ID NO: 57 - RNA, 79 nt, synthetic construct; identical to SEQ ID NO: 51
  source 1..79: mol_type = other RNA

SEQ ID NO: 58 - RNA, 79 nt, synthetic construct
  source 1..79: mol_type = other RNA
tcaatacagattatggcgcactacacaacccctatcacgtaagcattaagaaactcaagatcataatctgtattgattt

SEQ ID NO: 59 - RNA, 79 nt, synthetic construct; identical to SEQ ID NO: 53
  source 1..79: mol_type = other RNA

SEQ ID NO: 60 - RNA, 79 nt, synthetic construct
  source 1..79: mol_type = other RNA
ttgtttattgacatggcgcactacacaacccctatcacgtaagcattaagaaactcaagatcatgtcaataaacaattt

SEQ ID NO: 61 - RNA, 78 nt, synthetic construct
  source 1..78: mol_type = other RNA
tgatatacgaccagtgcgcactacacaacccctatcacgtaagcattaagaaactcaagatactggtctatatcattt

SEQ ID NO: 62 - RNA, 78 nt, synthetic construct
  source 1..78: mol_type = other RNA
tgataagaaatgactgcgcactacacaacccctatcacgtaagcattaagaaactcaagatagtcattcttatcattt

SEQ ID NO: 63 - RNA, 78 nt, synthetic construct
  source 1..78: mol_type = other RNA
cattagtatgttaaggcgcactacacaacccctatcacgtaagcattaagaaactcaagatcttaacaactaatgttt

SEQ ID NO: 64 - RNA, 78 nt, synthetic construct
  source 1..78: mol_type = other RNA
tcaatacagattatggcgcactacacaacccctatcacgtaagcattaagaaactcaagatcataatcgtattgattt

SEQ ID NO: 65 - RNA, 78 nt, synthetic construct
  source 1..78: mol_type = other RNA
ttatatacttcaagggcgcactacacaacccctatcacgtaagcattaagaaactcaagatccttgaatatataattt

SEQ ID NO: 66 - RNA, 78 nt, synthetic construct
  source 1..78: mol_type = other RNA
ttgtttattgacatggcgcactacacaacccctatcacgtaagcattaagaaactcaagatcatgtcataaacaattt

SEQ ID NO: 67 - RNA, 83 nt, synthetic construct
  source 1..83: mol_type = other RNA
tgatatacgaccagtgcgcactacacaacccctatcacgtaagcattaagaaactcaagatactggtctatatcattatgagc

SEQ ID NO: 68 - RNA, 83 nt, synthetic construct
  source 1..83: mol_type = other RNA
tgataagaaatgactgcgcactacacaacccctatcacgtaagcattaagaaactcaagatagtcattcttatcattcagaca

SEQ ID NO: 69 - RNA, 83 nt, synthetic construct
  source 1..83: mol_type = other RNA
cattagtatgttaaggcgcactacacaacccctatcacgtaagcattaagaaactcaagatcttaacaactaatgtttagtag

SEQ ID NO: 70 - RNA, 83 nt, synthetic construct
  source 1..83: mol_type = other RNA
tcaatacagattatggcgcactacacaacccctatcacgtaagcattaagaaactcaagatcataatcgtattgattctttca

SEQ ID NO: 71 - RNA, 83 nt, synthetic construct
  source 1..83: mol_type = other RNA
ttatatacttcaagggcgcactacacaacccctatcacgtaagcattaagaaactcaagatccttgaatatataattcatgcc

SEQ ID NO: 72 - RNA, 83 nt, synthetic construct
  source 1..83: mol_type = other RNA
ttgtttattgacatggcgcactacacaacccctatcacgtaagcattaagaaactcaagatcatgtcataaacaattatacaa

SEQ ID NO: 73 - RNA, 83 nt, synthetic construct; identical to SEQ ID NO: 67
  source 1..83: mol_type = other RNA

SEQ ID NO: 74 - RNA, 83 nt, synthetic construct
  source 1..83: mol_type = other RNA
tgataagaaatgactatccgaactacaacccctatcacgtaagcattaatcatccaacatgagtcattcttatcattcagaca

SEQ ID NO: 75 - RNA, 83 nt, synthetic construct
  source 1..83: mol_type = other RNA
cattagtatgttaagtgactcatcataacccctatcacgtaagcattaactactcaacgatcttaacaactaatgtttagtag

SEQ ID NO: 76 - RNA, 83 nt, synthetic construct
  source 1..83: mol_type = other RNA
tcaatacagattatgacagcatctataacccctatcacgtaagcattaatacaccacgacgcataatcgtattgattctttca

SEQ ID NO: 77 - RNA, 83 nt, synthetic construct
  source 1..83: mol_type = other RNA
ctatatacttcaaggaccatcactacaacccctatcacgtaagcattaacactactagcagccttgaatatatagttcatgcc

SEQ ID NO: 78 - RNA, 83 nt, synthetic construct
  source 1..83: mol_type = other RNA
ctgtttattgacatgtcacatctcacaacccctatcacgtaagcattaacacatcacgcatcatgtcataaacagttatacaa

SEQ ID NO: 79 - DNA, 23 bp, synthetic construct
  source 1..23: mol_type = other DNA
ctacctgcactgtaagcactttg

SEQ ID NO: 80 - DNA, 24 bp, synthetic construct
  source 1..24: mol_type = other DNA
ctacctgcactgtaagcaactttg

SEQ ID NO: 81 - DNA, 24 bp, synthetic construct
  source 1..24: mol_type = other DNA
ctacctgcactgtaagcagctttg

SEQ ID NO: 82 - DNA, 24 bp, synthetic construct
  source 1..24: mol_type = other DNA
ctacctgcactgtaagcacctttg

SEQ ID NO: 83 - DNA, 24 bp, synthetic construct
  source 1..24: mol_type = other DNA
ctacctgcactgtaagcatctttg

SEQ ID NO: 84 - DNA, 22 bp, synthetic construct
  source 1..22: mol_type = other DNA
ctacctgcactgatgcactttg

SEQ ID NO: 85 - DNA, 23 bp, synthetic construct
  source 1..23: mol_type = other DNA
ctacctgcactgatgcaactttg

SEQ ID NO: 86 - DNA, 23 bp, synthetic construct
  source 1..23: mol_type = other DNA
ctacctgcactgatgcagctttg

SEQ ID NO: 87 - DNA, 23 bp, synthetic construct
  source 1..23: mol_type = other DNA
ctacctgcactgatgcacctttg

SEQ ID NO: 88 - DNA, 23 bp, synthetic construct
  source 1..23: mol_type = other DNA
ctacctgcactgatgcatctttg

SEQ ID NO: 89 - DNA, 23 bp, synthetic construct
  source 1..23: mol_type = other DNA
ctacctgcactgtaagcacttag

SEQ ID NO: 90 - DNA, 23 bp, synthetic construct
  source 1..23: mol_type = other DNA
ctacctgcactgtaagcactatg

SEQ ID NO: 91 - DNA, 23 bp, synthetic construct
  source 1..23: mol_type = other DNA
ctacctgcactgtaagcacattg

SEQ ID NO: 92 - DNA, 23 bp, synthetic construct
  source 1..23: mol_type = other DNA
ctacctgcactgtaagcagtttg

SEQ ID NO: 93 - DNA, 23 bp, synthetic construct
  source 1..23: mol_type = other DNA
ctacctgcactgtaagctctttg

SEQ ID NO: 94 - DNA, 23 bp, synthetic construct
  source 1..23: mol_type = other DNA
ctacctgcactgtaaggactttg

SEQ ID NO: 95 - DNA, 23 bp, synthetic construct
  source 1..23: mol_type = other DNA
ctacctgcactgtaaccactttg

SEQ ID NO: 96 - DNA, 22 bp, synthetic construct
  source 1..22: mol_type = other DNA
ctacctgcactgatgcacttag

SEQ ID NO: 97 - DNA, 22 bp, synthetic construct
  source 1..22: mol_type = other DNA
ctacctgcactgatgcactatg

SEQ ID NO: 98 - DNA, 22 bp, synthetic construct
  source 1..22: mol_type = other DNA
ctacctgcactgatgcacattg

SEQ ID NO: 99 - DNA, 22 bp, synthetic construct
  source 1..22: mol_type = other DNA
ctacctgcactgatgcagtttg

SEQ ID NO: 100 - DNA, 22 bp, synthetic construct
  source 1..22: mol_type = other DNA
ctacctgcactgatgctctttg

SEQ ID NO: 101 - DNA, 22 bp, synthetic construct
  source 1..22: mol_type = other DNA
ctacctgcactgatggactttg

SEQ ID NO: 102 - DNA, 22 bp, synthetic construct
  source 1..22: mol_type = other DNA
ctacctgcactgatccactttg

SEQ ID NO: 103 - DNA, 23 bp, synthetic construct
  source 1..23: mol_type = other DNA
ctatctgcactagatgcacctta

SEQ ID NO: 104 - DNA, 24 bp, synthetic construct
  source 1..24: mol_type = other DNA
ctatctgcactagatgcaacctta

SEQ ID NO: 105 - DNA, 24 bp, synthetic construct
  source 1..24: mol_type = other DNA
ctatctgcactagatgcagcctta

SEQ ID NO: 106 - DNA, 24 bp, synthetic construct
  source 1..24: mol_type = other DNA
ctatctgcactagatgcaccctta

SEQ ID NO: 107 - DNA, 24 bp, synthetic construct
  source 1..24: mol_type = other DNA
ctatctgcactagatgcatcctta

SEQ ID NO: 108 - DNA, 22 bp, synthetic construct
  source 1..22: mol_type = other DNA
ctatctgcactactgcacctta

SEQ ID NO: 109 - DNA, 23 bp, synthetic construct
  source 1..23: mol_type = other DNA
ctatctgcactactgcaacctta

SEQ ID NO: 110 - DNA, 23 bp, synthetic construct
  source 1..23: mol_type = other DNA
ctatctgcactactgcagcctta

SEQ ID NO: 111 - DNA, 23 bp, synthetic construct
  source 1..23: mol_type = other DNA
ctatctgcactactgcaccctta

SEQ ID NO: 112 - DNA, 23 bp, synthetic construct
  source 1..23: mol_type = other DNA
ctatctgcactactgcatcctta

SEQ ID NO: 113 - DNA, 23 bp, synthetic construct
  source 1..23: mol_type = other DNA
ctatctgcactagatgcacctaa

SEQ ID NO: 114 - DNA, 23 bp, synthetic construct
  source 1..23: mol_type = other DNA
ctatctgcactagatgcaccata

SEQ ID NO: 115 - DNA, 23 bp, synthetic construct
  source 1..23: mol_type = other DNA
ctatctgcactagatgcacgtta

SEQ ID NO: 116 - DNA, 23 bp, synthetic construct
  source 1..23: mol_type = other DNA
ctatctgcactagatgcagctta

SEQ ID NO: 117 - DNA, 23 bp, synthetic construct
  source 1..23: mol_type = other DNA
ctatctgcactagatgctcctta

SEQ ID NO: 118 - DNA, 23 bp, synthetic construct
  source 1..23: mol_type = other DNA
ctatctgcactagatggacctta

SEQ ID NO: 119 - DNA, 23 bp, synthetic construct
  source 1..23: mol_type = other DNA
ctatctgcactagatccacctta

SEQ ID NO: 120 - DNA, 22 bp, synthetic construct
  source 1..22: mol_type = other DNA
ctatctgcactactgcacctaa

SEQ ID NO: 121 - DNA, 22 bp, synthetic construct
  source 1..22: mol_type = other DNA
ctatctgcactactgcaccata

SEQ ID NO: 122 - DNA, 22 bp, synthetic construct
  source 1..22: mol_type = other DNA
ctatctgcactactgcacgtta

SEQ ID NO: 123 - DNA, 22 bp, synthetic construct
  source 1..22: mol_type = other DNA
ctatctgcactactgcagctta

SEQ ID NO: 124 - DNA, 22 bp, synthetic construct
  source 1..22: mol_type = other DNA
ctatctgcactactgctcctta

SEQ ID NO: 125 - DNA, 22 bp, synthetic construct
  source 1..22: mol_type = other DNA
ctatctgcactactggacctta

SEQ ID NO: 126 - DNA, 22 bp, synthetic construct
  source 1..22: mol_type = other DNA
ctatctgcactactccacctta

SEQ ID NO: 127 - DNA, 22 bp, synthetic construct
  source 1..22: mol_type = other DNA
cgaccatggctgtagactgtta

SEQ ID NO: 128 - DNA, 23 bp, synthetic construct
  source 1..23: mol_type = other DNA
cgaccatggctgtagacatgtta

SEQ ID NO: 129 - DNA, 23 bp, synthetic construct
  source 1..23: mol_type = other DNA
cgaccatggctgtagacgtgtta

SEQ ID NO: 130 - DNA, 23 bp, synthetic construct
  source 1..23: mol_type = other DNA
cgaccatggctgtagacctgtta

SEQ ID NO: 131 - DNA, 23 bp, synthetic construct
  source 1..23: mol_type = other DNA
cgaccatggctgtagacttgtta

SEQ ID NO: 132 - DNA, 21 bp, synthetic construct
  source 1..21: mol_type = other DNA
cgaccatggctcagactgtta

SEQ ID NO: 133 - DNA, 22 bp, synthetic construct
  source 1..22: mol_type = other DNA
cgaccatggctcagacatgtta

SEQ ID NO: 134 - DNA, 22 bp, synthetic construct
  source 1..22: mol_type = other DNA
cgaccatggctcagacgtgtta

SEQ ID NO: 135 - DNA, 22 bp, synthetic construct
  source 1..22: mol_type = other DNA
cgaccatggctcagacctgtta

SEQ ID NO: 136 - DNA, 22 bp, synthetic construct
  source 1..22: mol_type = other DNA
cgaccatggctcagacttgtta

SEQ ID NO: 137 - DNA, 22 bp, synthetic construct
  source 1..22: mol_type = other DNA
cgaccatggctgtagactgtaa

SEQ ID NO: 138 - DNA, 22 bp, synthetic construct
  source 1..22: mol_type = other DNA
cgaccatggctgtagactgata

SEQ ID NO: 139 - DNA, 22 bp, synthetic construct
  source 1..22: mol_type = other DNA
cgaccatggctgtagactctta

SEQ ID NO: 140 - DNA, 22 bp, synthetic construct
  source 1..22: mol_type = other DNA
cgaccatggctgtagacagtta

SEQ ID NO: 141 - DNA, 22 bp, synthetic construct
  source 1..22: mol_type = other DNA
cgaccatggctgtagagtgtta

SEQ ID NO: 142 - DNA, 22 bp, synthetic construct
  source 1..22: mol_type = other DNA
cgaccatggctgtagtctgtta

SEQ ID NO: 143 - DNA, 22 bp, synthetic construct
  source 1..22: mol_type = other DNA
cgaccatggctgtacactgtta

SEQ ID NO: 144 - DNA, 21 bp, synthetic construct
  source 1..21: mol_type = other DNA
cgaccatggctcagactgtaa

SEQ ID NO: 145 - DNA, 21 bp, synthetic construct
  source 1..21: mol_type = other DNA
cgaccatggctcagactgata

SEQ ID NO: 146 - DNA, 21 bp, synthetic construct
  source 1..21: mol_type = other DNA
cgaccatggctcagactctta

SEQ ID NO: 147 - DNA, 21 bp, synthetic construct
  source 1..21: mol_type = other DNA
cgaccatggctcagacagtta

SEQ ID NO: 148 - DNA, 21 bp, synthetic construct
  source 1..21: mol_type = other DNA
cgaccatggctcagagtgtta

SEQ ID NO: 149 - DNA, 21 bp, synthetic construct
  source 1..21: mol_type = other DNA
cgaccatggctcagtctgtta

SEQ ID NO: 150 - DNA, 21 bp, synthetic construct
  source 1..21: mol_type = other DNA
cgaccatggctcacactgtta

SEQ ID NO: 151 - DNA, 24 bp, synthetic construct
  source 1..24: mol_type = other DNA
aacccctatcacgattagcattaa

SEQ ID NO: 152 - DNA, 25 bp, synthetic construct
  source 1..25: mol_type = other DNA
aacccctatcacgattagcaattaa

SEQ ID NO: 153 - DNA, 25 bp, synthetic construct
  source 1..25: mol_type = other DNA
aacccctatcacgattagcgattaa

SEQ ID NO: 154 - DNA, 25 bp, synthetic construct
  source 1..25: mol_type = other DNA
aacccctatcacgattagccattaa

SEQ ID NO: 155 - DNA, 25 bp, synthetic construct
  source 1..25: mol_type = other DNA
aacccctatcacgattagctattaa

SEQ ID NO: 156 - DNA, 23 bp, synthetic construct
  source 1..23: mol_type = other DNA
aacccctatcacgtaagcattaa

SEQ ID NO: 157 - DNA, 24 bp, synthetic construct
  source 1..24: mol_type = other DNA
aacccctatcacgtaagcaattaa

SEQ ID NO: 158 - DNA, 24 bp, synthetic construct
  source 1..24: mol_type = other DNA
aacccctatcacgtaagcgattaa

SEQ ID NO: 159 - DNA, 24 bp, synthetic construct
  source 1..24: mol_type = other DNA
aacccctatcacgtaagccattaa

SEQ ID NO: 160 - DNA, 24 bp, synthetic construct
  source 1..24: mol_type = other DNA
aacccctatcacgtaagctattaa

SEQ ID NO: 161 - DNA, 24 bp, synthetic construct
  source 1..24: mol_type = other DNA
aacccctatcacgattagcattta

SEQ ID NO: 162 - DNA, 24 bp, synthetic construct
  source 1..24: mol_type = other DNA
aacccctatcacgattagcataaa

SEQ ID NO: 163 - DNA, 24 bp, synthetic construct
  source 1..24: mol_type = other DNA
aacccctatcacgattagcaataa

SEQ ID NO: 164 - DNA, 24 bp, synthetic construct
  source 1..24: mol_type = other DNA
aacccctatcacgattagctttaa

SEQ ID NO: 165 - DNA, 24 bp, synthetic construct
  source 1..24: mol_type = other DNA
aacccctatcacgattaggattaa

SEQ ID NO: 166 - DNA, 24 bp, synthetic construct
  source 1..24: mol_type = other DNA
aacccctatcacgattaccattaa

SEQ ID NO: 167 - DNA, 24 bp, synthetic construct
  source 1..24: mol_type = other DNA
aacccctatcacgatttgcattaa

SEQ ID NO: 168 - DNA, 23 bp, synthetic construct
  source 1..23: mol_type = other DNA
aacccctatcacgtaagcattta

SEQ ID NO: 169 - DNA, 23 bp, synthetic construct
  source 1..23: mol_type = other DNA
aacccctatcacgtaagcataaa

SEQ ID NO: 170 - DNA, 23 bp, synthetic construct
  source 1..23: mol_type = other DNA
aacccctatcacgtaagcaataa

SEQ ID NO: 171 - DNA, 23 bp, synthetic construct
  source 1..23: mol_type = other DNA
aacccctatcacgtaagctttaa

SEQ ID NO: 172 - DNA, 23 bp, synthetic construct
  source 1..23: mol_type = other DNA
aacccctatcacgtaaggattaa

SEQ ID NO: 173 - DNA, 23 bp, synthetic construct
  source 1..23: mol_type = other DNA
aacccctatcacgtaaccattaa

SEQ ID NO: 174 - DNA, 23 bp, synthetic construct
  source 1..23: mol_type = other DNA
aacccctatcacgtatgcattaa

SEQ ID NO: 175 - DNA, 492 bp, synthetic construct
  source 1..492: mol_type = other DNA
tgatatacgaccagttcttattctcccgaccatggctgtagactgttaaatacttcaaatactggtctatatcattatgagctgataagaaatgacttcaaccaaatccgaccatggctgtagactgttaacaatatcccttagtcattcttatcattcagacacattagtatgttaagtatatcaaattcgaccatggctgtagactgttaatattctctcctcttaacaactaatgtttagtagtcaatacagattatgtacacataattcgaccatggctgtagactgttaataatcctatttcataatcgtattgattctttcactatatacttcaaggtattctcttctcgaccatggctgtagactgttaacacaacacactccttgaatatatagttcatgccctgtttattgacatgtactcatatttcgaccatggctgtagactgttaatttatcctattcatgtcataaacagttatacaa

SEQ ID NO: 176 - DNA, 498 bp, synthetic construct
  source 1..498: mol_type = other DNA
tgatatacgaccagtatagactacaacgaccatggctgtagacatgttagaaactcaagatactggtctatatcattatgagctgataagaaatgactcaacttacgtacgaccatggctgtagacatgttacaatatcacctcagtcattcttatcacttagacacattagtatgttaagctcatccttatcgaccatggctgtagacatgttacactcaaatcatcttaacaactaatgtttagtagtcaatacagattatgactctatcaaacgaccatggctgtagacatgttagcaacatcatcacataatcgtattgacatgcaatctatatacttcaaggaagatgactaacgaccatggctgtagacatgttacactgctagtaaccttgaatatatagctgtagttctgtttattgacatgtcaacatcaatcgaccatggctgtagacatgttacactttacctaacatgtcataaacagtaactgaa

SEQ ID NO: 177 - DNA, 498 bp, synthetic construct
  source 1..498: mol_type = other DNA
tgatatacgaccagtatagactacaacgaccatggctgtagacgtgttagaaactcaagatactggtctatatcattatgagctgataagaaatgactcaacttacgtacgaccatggctgtagacgtgttacaatatcacctcagtcattcttatcacttagacacattagtatgttaagctcatccttatcgaccatggctgtagacgtgttacactcaaatcatcttaacaactaatgtttagtagtcaatacagattatgactctatcaaacgaccatggctgtagacgtgttagcatcatcatcacataatcgtattgacatgcaatctatatacttcaaggaagatgactaacgaccatggctgtagacgtgttacactgctagtaaccttgaatatatagctgaagttctgtttattgacatgtcaacatcaatcgaccatggctgtagacgtgttacactttacctaacatgtcataaacagtaaccgaa

SEQ ID NO: 178 - DNA, 498 bp, synthetic construct
  source 1..498: mol_type = other DNA
tgatatacgaccagtcacatacctcccgaccatggctgtagacctgttaatactctcacatactggtctatatcattatgagctgataagaaatgactactattcacaacgaccatggctgtagacctgttaaatccaaattcaagtcattcttatcattcagacacattagtatgttaagcctcacttccccgaccatggctgtagacctgttaaacattccacctcttaacaactaatgtttagtagtcaatacagattatgcaccctatcaacgaccatggctgtagacctgttaaacatacacactcataatcgtattgattctttcactatatacttcaaggccttcttctatcgaccatggctgtagacctgttaacctcactaataccttgaatatatagttcatgccctgtttattgacatgacactccctatcgaccatggctgtagacctgttacttcttctatcacatgtcataaacagttatacaa

SEQ ID NO: 179 - DNA, 498 bp, synthetic construct
  source 1..498: mol_type = other DNA
tgatatacgaccagttacttcactctcgaccatggctgtagacttgttacttacttcaactactggtctatatcatcttgagctgataagaaatgactcatactaacttcgaccatggctgtagacttgttaaccttatcattcagtcattcttatcacttagacacattagtatgttaagctcttactaatcgaccatggctgtagacttgttactaacttaccctcttaacaactaatgtttagtagtcaatacagattatgtcattccctatcgaccatggctgtagacttgttaactcttacctcacataatcgtattgacatgcaatctatatacttcaaggtttcatcccttcgaccatggctgtagacttgttaaatcccttcattccttgaatatatagctttagttctgtttattgacatgtccactttaatcgaccatggctgtagacttgttaccaactttcaatcatgtcataaacagtaaccgaa

SEQ ID NO: 180 - DNA, 486 bp, synthetic construct
  source 1..486: mol_type = other DNA
tgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagacacattagtatgttaagctcatccttatcgaccatggctcagactgttacactcaaatcatcttaacaactaatgtttagtagtcaatacagattatgactctatcaaacgaccatggctcagactgttagcaacatcatcacataatcgtattgacttgcaatctatatacttcaaggaagatcactaacgaccatggctcagactgttacactactaccaaccttgaatatatagccgtagttctgtttattgacatgtcaacatcaatcgaccatggctcagactgttacactttacctaacatgtcataaacagtaacagaa

SEQ ID NO: 181 - DNA, 492 bp, synthetic construct
  source 1..492: mol_type = other DNA
tgatatacgaccagtatagactacaacgaccatggctcagacatgttagaaactcaagatactggtctatatcattatgagctgataagaaatgactcaacttacgtacgaccatggctcagacatgttacaatatcacctcagtcattcttatcacttagacacattagtatgttaagctcatccttatcgaccatggctcagacatgttacactcaaatcatcttaacaactaatgtttagtagtcaatacagattatgactctatcaaacgaccatggctcagacatgttagcaacatcatcacataatcgtattgacttgcaatctatatacttcaaggaagatcactaacgaccatggctcagacatgttacactactaccaaccttgaatatatagccgtagttctgtttattgacatgtcaacatcaatcgaccatggctcagacatgttacactttacctaacatgtcataaacagtaacagaa

SEQ ID NO: 182 - DNA, 492 bp, synthetic construct
  source 1..492: mol_type = other DNA
tgatatacgaccagtatagactacaacgaccatggctcagacgtgttagaaactcaagatactggtctatatcattatgagctgataagaaatgactcaacttacgtacgaccatggctcagacgtgttacaatatcacctcagtcattcttatcacttagacacattagtatgttaagctcatccttatcgaccatggctcagacgtgttacactcaaatcatcttaacaactaatgtttagtagtcaatacagattatgactctatcaaacgaccatggctcagacgtgttagcaacatcatcacataatcgtattgacttgcaatctatatacttcaaggaagatcactaacgaccatggctcagacgtgttacactactaccaaccttgaatatatagccgtagttctgtttattgacatgtcaacatcaatcgaccatggctcagacgtgttacactttacctaacatgtcataaacagtaacagaa

SEQ ID NO: 183 - DNA, 492 bp, synthetic construct
  source 1..492: mol_type = other DNA
tgatatacgaccagtatagactacaacgaccatggctcagacctgttagaaactcaagatactggtctatatcattatgagctgataagaaatgactcaacttacgtacgaccatggctcagacctgttacaatatcacctcagtcattcttatcacttagacacattagtatgttaagctcatccttatcgaccatggctcagacctgttacactcaaatcatcttaacaactaatgtttagtagtcaatacagattatgactctatcaaacgaccatggctcagacctgttagcaacatcatcacataatcgtattgacttgcaatctatatacttcaaggaagatcactaacgaccatggctcagacctgttacactactaccaaccttgaatatatagccgtagttctgtttattgacatgtcaacatcaatcgaccatggctcagacctgttacactttacctaacatgtcataaacagtaacagaa

SEQ ID NO: 184 - DNA, 492 bp, synthetic construct
  source 1..492: mol_type = other DNA
tgatatacgaccagtatagactacaacgaccatggctcagacttgttagaaactcaagatactggtctatatcattatgagctgataagaaatgactcaacttacctacgaccatggctcagacttgttacaatatcacctcagtcattcttatcacttagacacattagtatgttaagcttacgcataccgaccatggctcagacttgttacactctaatcctcttaacaactaatgtttagtagtcaatacagattatgactctatcaaacgaccatggctcagacttgttagcaacatcatcacataatcgtattgacttgcaatctatatacttcaaggatcctcacactcgaccatggctcagacttgttacactacatccatccttgaatatatagccgtagttctgtttattgacatgtcaacatcaatcgaccatggctcagacttgttacactttacctaacatgtcataaacagtaacagaa

SEQ ID NO: 185 - DNA, 510 bp, synthetic construct
  source 1..510: mol_type = other DNA
tgatatacgaccagtgcgcactacacaacccctatcacgattagcaattaagaaactcaagatactggtctatatcattatgagctgataagaaatgactactttaactacaacccctatcacgattagcaattaagaacacgacatcagtcattcttatcattcagacacattagtatgttaagttaagcatcataacccctatcacgattagcaattaacaacgaaacgatcttaacaactaatgtttagtagtcaatacagattatgacagcatctataacccctatcacgattagcaattaatacaccacgacgcataatcgtattgattctttcactatatacttcaaggaccatcactacaacccctatcacgattagcaattaacactactagcagccttgaatatatagttcatgccctgtttattgacatgtcacatctcacaacccctatcacgattagcaattaacacatcacgcatcatgtcataaacagttatacaa

SEQ ID NO: 186 - DNA, 510 bp, synthetic construct
  source 1..510: mol_type = other DNA
tgatatacgaccagttcatactacacaacccctatcacgattagcgattaagaaactcaatatactggtctatatcattatgagctgataagaaatgactacaccgacataaacccctatcacgattagcgattaacctacagagattagtcattcttatcattcagacacattagtatgttaagtgactcatcataacccctatcacgattagcgattaaattcagaacgatcttaacaactaatgtttagtagtcaatacagattatgacagcaactataacccctatcacgattagcgattaatacaccacgaatcataatcgtattgattctttcactatatacttcaaggacatacctatcaacccctatcacgattagcgattaacaccatgaccagccttgaatatatagttcatgccctgtttattgacatgtcacatctcacaacccctatcacgattagcgattaacactccactcatcatgtcataaacagttatacaa

SEQ ID NO: 187 - DNA, 510 bp, synthetic construct
  source 1..510: mol_type = other DNA
tgatatacgaccagtgcgcactacacaacccctatcacgattagccattaagaaactcaagatactggtctatatcattatgagctgataagaaatgactactttaactacaacccctatcacgattagccattaagaacacgacatcagtcattcttatcattcagacacattagtatgttaagttaagcatcataacccctatcacgattagccattaacaacgaaacgatcttaacaactaatgtttagtagtcaatacagattatgacagcatctataacccctatcacgattagccattaatacaccacgacgcataatcgtattgattctttcactatatacttcaaggaccatcactacaacccctatcacgattagccattaacactactagcagccttgaatatatagttcatgccctgtttattgacatgtcacatctcacaacccctatcacgattagccattaacacatcacgcatcatgtcataaacagttatacaa

SEQ ID NO: 188 - DNA, 510 bp, synthetic construct
  source 1..510: mol_type = other DNA
tgatatacgaccagtgcgcactacacaacccctatcacgattagctattaagaaactcaagatactggtctatatcattatgagctgataagaaatgactactttaactacaacccctatcacgattagctattaagaacacgacatcagtcattcttatcattcagacacattagtatgttaagttaccgatcataacccctatcacgattagctattaacttacaaacgatcttaacaactaatgtttagtagtcaatacagattatgacatcacgcataacccctatcacgattagctattaatacaccacgacgcataatcgtattgattctttcactatatacttcaaggactctatctacaacccctatcacgattagctattaacacttcacgcctccttgaatatatagttcatgccctgtttattgacatgtcacatctcacaacccctatcacgattagctattaacacatcacgcatcatgtcataaacagttatacaa

SEQ ID NO: 189 - DNA, 498 bp, synthetic construct
  source 1..498: mol_type = other DNA
tgatatacgaccagtgcgcactacacaacccctatcacgtaagcattaagaaactcaagatactggtctatatcattatgagctgataagaaatgactatccgaactacaacccctatcacgtaagcattaatcatccaacatgagtcattcttatcattcagacacattagtatgttaagtgactcatcataacccctatcacgtaagcattaactactcaacgatcttaacaactaatgtttagtagtcaatacagattatgacagcatctataacccctatcacgtaagcattaatacaccacgacgcataatcgtattgattctttcactatatacttcaaggaccatcactacaacccctatcacgtaagcattaacactactagcagccttgaatatatagttcatgccctgtttattgacatgtcacatctcacaacccctatcacgtaagcattaacacatcacgcatcatgtcataaacagttatacaa

SEQ ID NO: 190 - DNA, 504 bp, synthetic construct
  source 1..504: mol_type = other DNA
tgatatacgaccagtgcgcactacacaacccctatcacgtaagcaattaagaaactcaagatactggtctatatcattatgagctgataagaaatgactatccgaactacaacccctatcacgtaagcaattaatcatccaacatgagtcattcttatcattcagacacattagtatgttaagtgactcatcataacccctatcacgtaagcaattaactactcaacgatcttaacaactaatgtttagtagtcaatacagattatgacagcatctataacccctatcacgtaagcaattaatacaccacgacgcataatcgtattgattctttcactatatacttcaaggaccatcactacaacccctatcacgtaagcaattaacactactagcagccttgaatatatagttcatgccctgtttattgacatgtcacatctcacaacccctatcacgtaagcaattaacacatcacgcatcatgtcataaacagttatacaa

SEQ ID NO: 191 - DNA, 504 bp, synthetic construct
  source 1..504: mol_type = other DNA
tgatatacgaccagttcagactacacaacccctatcacgtaagcgattaagaaactcaatatactggtctatatcattatgagctgataagaaatgactacaccgacataaacccctatcacgtaagcgattaacctacagagattagtcattcttatcattcagacacattagtatgttaagtgactcagcataacccctatcacgtaagcgattaaattcagaacaatcttaacaactaatgtttagtagtcaatacagattatgacagcaactataacccctatcacgtaagcgattaatacaccacgaatcataatcgtattgattctttcactatatacttcaaggacatacctatcaacccctatcacgtaagcgattaacaccatcaccagccttgaatatatagttcatgccctgtttattgacatgtcacatctcacaacccctatcacgtaagcgattaacactccactcatcatgtcataaacagttatacaa

SEQ ID NO: 192 - DNA, 504 bp, synthetic construct
  source 1..504: mol_type = other DNA
tgatatacgaccagtgcgcactacacaacccctatcacgtaagccattaagaaactcaagatactggtctatatcattatgagctgataagaaatgactatccgaactacaacccctatcacgtaagccattaatcatccaacatgagtcattcttatcattcagacacattagtatgttaagtgactcatcataacccctatcacgtaagccattaactactcaacgatcttaacaactaatgtttagtagtcaatacagattatgacagcatctataacccctatcacgtaagccattaatacaccacgacgcataatcgtattgattctttcactatatacttcaaggaccatcactacaacccctatcacgtaagccattaacactactagcagccttgaatatatagttcatgccctgtttattgacatgtcacatctcacaacccctatcacgtaagccattaacacatcacgcatcatgtcataaacagttatacaa

SEQ ID NO: 193 - DNA, 504 bp, synthetic construct
  source 1..504: mol_type = other DNA
tgatatacgaccagtgcgcactacacaacccctatcacgtaagctattaagaaactcaagatactggtctatatcattatgagctgataagaaatgactatccgaactacaacccctatcacgtaagctattaatcatccaacatgagtcattcttatcattcagacacattagtatgttaagtgactcatcataacccctatcacgtaagctattaactactcaacgatcttaacaactaatgtttagtagtcaatacagattatgacaccatctataacccctatcacgtaagctattaatacaccacgacgcataatcgtattgattctttcactatatacttcaaggaccatcacaacaacccctatcacgtaagctattaacacgaccacgagccttgaatatatagttcatgccctgtttattgacatgtcacatctcacaacccctatcacgtaagctattaacacatcacgcatcatgtcataaacagttatacaa

SEQ ID NO: 194 - DNA, 498 bp, synthetic construct
  source 1..498: mol_type = other DNA
tgatatacgaccagtccgcactacacctatctgcactagatgcaccttataaactcagaatactggtctatatcattatgagctgataagaaatgactagctcaagtccctatctgcactagatgcaccttaataagtcctccgagtcattcttatcattcagacacattagtatgttaagtcactcacgatctatctgcactagatgcaccttactactcaactatcttaacaactaatgtttagtagtcaatacagattatgacagcatagatctatctgcactagatgcaccttatacaccacgacgcataatcgtattgattctttcactatatacttcaaggaccatcactacctatctgcactagatgcaccttacactactaccagccttgaatatatagttcatgccctgtttattgacatgtcacatctcacctatctgcactagatgcaccttacacatcacgcatcatgtcataaacagttatacaa

SEQ ID NO: 195 - DNA, 504 bp, synthetic construct
  source 1..504: mol_type = other DNA
tgatatacgaccagtccgcactacacctatctgcactagatgcaaccttataaactcagaatactggtctatatcattatgagctgataagaaatgactagctcaagtccctatctgcactagatgcaaccttaataagtcctccgagtcattcttatcattcagacacattagtatgttaagtcactcacgatctatctgcactagatgcaaccttactactcaactatcttaacaactaatgtttagtagtcaatacagattatgacagcatagatctatctgcactagatgcaaccttatacaccacgacgcataatcgtattgattctttcactatatacttcaaggaccatcactacctatctgcactagatgcaaccttacactactaccagccttgaatatatagttcatgccctgtttattgacatgtcacatctcacctatctgcactagatgcaaccttacacatcacgcatcatgtcataaacagttatacaa

SEQ ID NO: 196 - DNA, 504 bp, synthetic construct
  source 1..504: mol_type = other DNA
tgatatacgaccagtccgcactacacctatctgcactagatgcagccttataaactcagaatactggtctatatcattatgagctgataagaaatgactagctcaagtccctatctgcactagatgcagccttaataagtcctccgagtcattcttatcattcagacacattagtatgttaagtcactcacgatctatctgcactagatgcagccttactactcaactatcttaacaactaatgtttagtagtcaatacagattatgacagcatagatctatctgcactagatgcagccttatacaccacgacgcataatcgtattgattctttcactatatacttcaaggaccatcactacctatctgcactagatgcagccttacactactaccagccttgaatatatagttcatgccctgtttattgacatgtcacatctcacctatctgcactagatgcagccttacacatcacgcatcatgtcataaacagttatacaa

SEQ ID NO: 197 - DNA, 504 bp, synthetic construct
  source 1..504: mol_type = other DNA
tgatatacgaccagtccgcactacacctatctgcactagatgcacccttataaactcagaatactggtctatatcattatgagctgataagaaatgactagctcaagtccctatctgcactagatgcacccttaataagtcctccgagtcattcttatcattcagacacattagtatgttaagtcactcacgatctatctgcactagatgcacccttactactcaactatcttaacaactaatgtttagtagtcaatacagattatgacagcatagatctatctgcactagatgcacccttatacaccacgacgcataatcgtattgattctttcactatatacttcaaggaccatcactacctatctgcactagatgcacccttacactactaccagccttgaatatatagttcatgccctgtttattgacatgtcacatctcacctatctgcactagatgcacccttacacatcacgcatcatgtcataaacagttatacaa

SEQ ID NO: 198 - DNA, 504 bp, synthetic construct
  source 1..504: mol_type = other DNA
tgatatacgaccagtccgcactacacctatctgcactagatgcatccttataaactcagaatactggtctatatcattatgagctgataagaaatgactagctcaagtccctatctgcactagatgcatccttaataagtcctccgagtcattcttatcattcagacacattagtatgttaagtcactcactatctatctgcactagatgcatccttactactcaactatcttaacaactaatgtttagtagtcaatacagattatgacagcatagacctatctgcactagatgcatccttatacaccacgacgcataatcgtattgattctttcactatatacttcaaggaccatcactacctatctgcactagatgcatccttacactactaccagccttgaatatatagttcatgccctgtttattgacatgtcacatctcacctatctgcactagatgcatccttacacatcacgcatcatgtcataaacagttatacaa

SEQ ID NO: 199 - DNA, 492 bp, synthetic construct
  source 1..492: mol_type = other DNA
tgatatacgaccagtccgcactacacctatctgcactactgcaccttataaactcagaatactggtctatatcattatgagctgataagaaatgacttcttcaagtccctatctgcactactgcaccttaataagtcctcctagtcattcttatcattcagacacattagtatgttaagtgactcatcatctatctgcactactgcaccttactactcaacgatcttaacaactaatgtttagtagtcaatacagattatgacagcatctatctatctgcactactgcaccttatacaccacgacgcataatcgtattgattctttcactatatacttcaaggaccatcactacctatctgcactactgcaccttacactactagcagccttgaatatatagttcatgccctgtttattgacatgtcacatctcacctatctgcactactgcaccttacacatcacgcatcatgtcataaacagttatacaa

SEQ ID NO: 200 - DNA, 498 bp, synthetic construct
  source 1..498: mol_type = other DNA
tgatatacgaccagtccgcactacacctatctgcactactgcaaccttataaactcagaatactggtctatatcattatgagctgataagaaatgacttcttcaagtccctatctgcactactgcaaccttaataagtcctcctagtcattcttatcattcagacacattagtatgttaagtcactcatcatctatctgcactactgcaaccttactactcaactgtcttaacaactaatgtttagtagtcaatacagattatgacagcatagatctatctgcactactgcaaccttatacaccacgacgcataatcgtattgattctttcactatatacttcaaggaccatcactacctatctgcactactgcaaccttacactactaccagccttgaatatatagttcatgccctgtttattgacatgtcacatctcacctatctgcactactgcaaccttacacatcacgcatcatgtcataaacagttatacaa

SEQ ID NO: 201 - DNA, 498 bp, synthetic construct
  source 1..498: mol_type = other DNA
tgatatacgaccagtccgcactacacctatctgcactactgcagccttataaactcagaatactggtctatatcattatgagctgataagaaatgacttcttcaagtccctatctgcactactgcagccttaataagtcctcctagtcattcttatcattcagacacattagtatgttaagtcactcatcatctatctgcactactgcagccttactactcaactgtcttaacaactaatgtttagtagtcaatacagattatgacagcatagatctatctgcactactgcagccttatacaccacgacgcataatcgtattgattctttcactatatacttcaaggaccatcactacctatctgcactactgcagccttacactactaccagccttgaatatatagttcatgccctgtttattgacatgtcacatctcacctatctgcactactgcagccttacacatcacgcatcatgtcataaacagttatacaa

SEQ ID NO: 202 - DNA, 498 bp, synthetic construct
  source 1..498: mol_type = other DNA
tgatatacgaccagtccgcactacacctatctgcactactgcacccttataaactcagaatactggtctatatcattatgagctgataagaaatgacttcttcaagtccctatctgcactactgcacccttaataagtcctcctagtcattcttatcattcagacacattagtatgttaagtcactcatcatctatctgcactactgcacccttactactcaactgtcttaacaactaatgtttagtagtcaatacagattatgacagcatagatctatctgcactactgcacccttatacaccacgacgcataatcgtattgattctttcactatatacttcaaggaccatcactacctatctgcactactgcacccttacactactaccagccttgaatatatagttcatgccctgtttattgacatgtcacatctcacctatctgcactactgcacccttacacatcacgcatcatgtcataaacagttatacaa

SEQ ID NO: 203 - DNA, 498 bp, synthetic construct
  source 1..498: mol_type = other DNA
tgatatacgaccagtccgcactacacctatctgcactactgcatccttataaactcagaatactggtctatatcattatgagctgataagaaatgacttcttcaagtccctatctgcactactgcatccttaataagtcctcctagtcattcttatcattcagacacattagtatgttaagtcactcatcatctatctgcactactgcatccttactactcaactgtcttaacaactaatgtttagtagtcaatacagattatgacagcatagatctatctgcactactgcatccttatacaccacgacgcataatcgtattgattctttcactatatacttcaaggaccatcactacctatctgcactactgcatccttacactactaccagccttgaatatatagttcatgccctgtttattgacatgtcacatctcacctatctgcactactgcatccttacacatcacgcatcatgtcataaacagttatacaa

SEQ ID NO: 204 - DNA, 498 bp, synthetic construct
  source 1..498: mol_type = other DNA
tgatatacgaccagtcctcactactactacctgcactgtaagcactttgacttcgccgattactggtctatatcattatgagctgataagaaatgactatcctgactacctacctgcactgtaagcactttgacatcctacctgagtcattcttatcattcagacacattagtatgttaagtcaagccacatctacctgcactgtaagcactttgctaagcgacactcttaacaactaatgtttagtagtcaatacagattatgacttccgatctctacctgcactgtaagcactttgtcctattacgtccataatcgtattgattctttcactatatacttcaaggaccatcacgaactacctgcactgtaagcactttgacctactaccatccttgaatatatagttcatgccctgtttattgacatgtcactccatacctacctgcactgtaagcactttgaccattcctcttcatgtcataaacagttatacaa

SEQ ID NO: 205 - DNA, 504 bp, synthetic construct
  source 1..504: mol_type = other DNA
tgatatacgaccagtcctcactactactacctgcactgtaagcaactttgacttcgccgattactggtctatatcattatgagctgataagaaatgactatcctgactacctacctgcactgtaagcaactttgacatcctacctgagtcattcttatcattcagacacattagtatgttaagtcaagccacatctacctgcactgtaagcaactttgctaagcgacactcttaacaactaatgtttagtagtcaatacagattatgacttccgatctctacctgcactgtaagcaactttgtcctattacgtccataatcgtattgattctttcactatatacttcaaggaccatcacgaactacctgcactgtaagcaactttgacctactaccatccttgaatatatagttcatgccctgtttattgacatgtcactccatacctacctgcactgtaagcaactttgaccattcctcttcatgtcataaacagttatacaa

SEQ ID NO: 206 - DNA, 504 bp, synthetic construct
  source 1..504: mol_type = other DNA
tgatatacgaccagtcctcactactactacctgcactgtaagcagctttgacttcgccgattactggtctatatcattatgagctgataagaaatgactatcctgactacctacctgcactgtaagcagctttgacatcctacctgagtcattcttatcattcagacacattagtatgttaagtcaagccacatctacctgcactgtaagcagctttgctaagcgacactcttaacaactaatgtttagtagtcaatacagattatgacttccgatctctacctgcactgtaagcagctttgtcctattacgtccataatcgtattgattctttcactatatacttcaaggaccatcacgaactacctgcactgtaagcagctttgacctactaccatccttgaatatatagttcatgccctgtttattgacatgtcactccatacctacctgcactgtaagcagctttgaccattcctcttcatgtcataaacagttatacaa

SEQ ID NO: 207 - DNA, 504 bp, synthetic construct
  source 1..504: mol_type = other DNA
tgatatacgaccagtcctcactactactacctgcactgtaagcacctttgacttcgccgattactggtctatatcattatgagctgataagaaatgactatcctgactacctacctgcactgtaagcacctttgacatcctacctgagtcattcttatcattcagacacattagtatgttaagtcaagccacatctacctgcactgtaagcacctttgctaagcgacactcttaacaactaatgtttagtagtcaatacagattatgacttccgatctctacctgcactgtaagcacctttgtcctattacgtccataatcgtattgattctttcactatatacttcaaggaccatcacgaactacctgcactgtaagcacctttgacctactaccatccttgaatatatagttcatgccctgtttattgacatgtcactccatacctacctgcactgtaagcacctttgaccattcctcttcatgtcataaacagttatacaa

SEQ ID NO: 208 - DNA, 504 bp, synthetic construct
  source 1..504: mol_type = other DNA
tgatatacgaccagtcctcactactactacctgcactgtaagcatctttgacttcgccgattactggtctatatcattatgagctgataagaaatgactatcctgactacctacctgcactgtaagcatctttgacatcctacctgagtcattcttatcattcagacacattagtatgttaagtcatgacacacctacctgcactgtaagcatctttgacatccgacactcttaacaactaatgtttagtagtcaatacagattatgacttccgatctctacctgcactgtaagcatctttgtcctattacgtccataatcgtattgattctttcactatatacttcaaggaccatcacgaactacctgcactgtaagcatctttgacctactaccatccttgaatatatagttcatgccctgtttattgacatgtcactccatacctacctgcactgtaagcatctttgaccattcctcttcatgtcataaacagttatacaa

SEQ ID NO: 209 - DNA, 492 bp, synthetic construct
  source 1..492: mol_type = other DNA
tgatatacgaccagtccgcactacacctacctgcactgatgcactttgtaaactcctgatactggtctatatcattatgagctgataagaaatgactatccgaactacctacctgcactgatgcactttgccatccaacctgagtcattcttatcattcagacacattagtatgttaagtgactcctcatctacctgcactgatgcactttgctactcaactatcttaacaactaatgtttagtagtcaatacagattatgacatcatatatctacctgcactgatgcactttgcccacctcgacgcataatcgtattgattctttcactatatacttcaaggaccatcactatctacctgcactgatgcactttgtcctactaccagccttgaatatatagttcatgccctgtttattgacatgtcacatctcacctacctgcactgatgcactttgcataacactcatcatgtcataaacagttatacaa

SEQ ID NO: 210 - DNA, 498 bp, synthetic construct
  source 1..498: mol_type = other DNA
tgatatacgaccagtccatacgacacctacctgcactgatgcaactttgctaactgatcatactggtctatatcattatgagctgataagaaatgactatccgaactacctacctgcactgatgcaactttgacatgctgactgagtcattcttatcattcagacacattagtatgttaagtgactccacatctacctgcactgatgcaactttgctacgcctctatcttaacaactaatgtttagtagtcaatacagattatgactttcacgccctacctgcactgatgcaactttgactactttaccgcataatcgtattgattctttcactatatacttcaaggagtctcacgctctacctgcactgatgcaactttgtactactaccagccttgaatatatagttcatgccctgtttattgacatgcgacaccactcctacctgcactgatgcaactttgctgaacactgatcatgtcataaacagttatacaa

SEQ ID NO: 211 - DNA, 498 bp, synthetic construct
  source 1..498: mol_type = other DNA
tgatatacgaccagtccatacgacacctacctgcactgatgcagctttgctaactgatcatactggtctatatcattatgagctgataagaaatgactatccgaactacctacctgcactgatgcagctttgacatgctgactgagtcattcttatcattcagacacattagtatgttaagtgactccacatctacctgcactgatgcagctttgctacgcctctatcttaacaactaatgtttagtagtcaatacagattatgactttcacgccctacctgcactgatgcagctttgactactttaccgcataatcgtattgattctttcactatatacttcaaggagtctcacgctctacctgcactgatgcagctttgtactactaccagccttgaatatatagttcatgccctgtttattgacatgcgacaccactcctacctgcactgatgcagctttgctgaacactgatcatgtcataaacagttatacaa

SEQ ID NO: 212 - DNA, 498 bp, synthetic construct
  source 1..498: mol_type = other DNA
tgatatacgaccagtccatacgacacctacctgcactgatgcacctttgctaactgatcatactggtctatatcattatgagctgataagaaatgactatccgaactacctacctgcactgatgcacctttgacatgctgactgagtcattcttatcattcagacacattagtatgttaagtgactccacatctacctgcactgatgcacctttgctacgcctctatcttaacaactaatgtttagtagtcaatacagattatgactttcacgccctacctgcactgatgcacctttgactactttaccgcataatcgtattgattctttcactatatacttcaaggagtctcacgctctacctgcactgatgcacctttgtactactaccagccttgaatatatagttcatgccctgtttattgacatgcgacaccactcctacctgcactgatgcacctttgctgaacactgatcatgtcataaacagttatacaa

SEQ ID NO: 213 - DNA, 498 bp, synthetic construct
  source 1..498: mol_type = other DNA
tgatatacgaccagtcctcactactactacctgcactgatgcatctttgacttcgccgattactggtctatatcattatgagctgataagaaatgactatcctgactacctacctgcactgatgcatctttgacctgctacctgagtcattcttatcattcagacacattagtatgttaagtcaagccacatctacctgcactgatgcatctttgctaagcgacactcttaacaactaatgtttagtagtcaatacagattatgacttccgatctctacctgcactgatgcatctttgtcctattacgtccataatcgtattgattctttcactatatacttcaaggaccttcacgaactacctgcactgatgcatctttgacctactaccatccttgaatatatagttcatgccctgtttattgacatgtcactccatacctacctgcactgatgcatctttgaccattcctcttcatgtcataaacagttatacaa

SEQ ID NO: 214 - DNA, 495 bp, synthetic construct
  source 1..495: mol_type = other DNA
gggtgatatacgaccagttcttattctcccgaccatggctgtagactgttaaatacttcaaatactggtctatatcattatgagctgataagaaatgacttcaaccaaatccgaccatggctgtagactgttaacaatatcccttagtcattcttatcattcagacacattagtatgttaagtatatcaaattcgaccatggctgtagactgttaatattctctcctcttaacaactaatgtttagtagtcaatacagattatgtacacataattcgaccatggctgtagactgttaataatcctatttcataatcgtattgattctttcactatatacttcaaggtattctcttctcgaccatggctgtagactgttaacacaacacactccttgaatatatagttcatgccctgtttattgacatgtactcatatttcgaccatggctgtagactgttaatttatcctattcatgtcataaacagttatacaa

SEQ ID NO: 215 - DNA, 501 bp, synthetic construct
  source 1..501: mol_type = other DNA
gggtgatatacgaccagtatagactacaacgaccatggctgtagacatgttagaaactcaagatactggtctatatcattatgagctgataagaaatgactcaacttacgtacgaccatggctgtagacatgttacaatatcacctcagtcattcttatcacttagacacattagtatgttaagctcatccttatcgaccatggctgtagacatgttacactcaaatcatcttaacaactaatgtttagtagtcaatacagattatgactctatcaaacgaccatggctgtagacatgttagcaacatcatcacataatcgtattgacatgcaatctatatacttcaaggaagatgactaacgaccatggctgtagacatgttacactgctagtaaccttgaatatatagctgtagttctgtttattgacatgtcaacatcaatcgaccatggctgtagacatgttacactttacctaacatgtcataaacagtaactgaa

SEQ ID NO: 216 - DNA, 501 bp, synthetic construct
  source 1..501: mol_type = other DNA
gggtgatatacgaccagtatagactacaacgaccatggctgtagacgtgttagaaactcaagatactggtctatatcattatgagctgataagaaatgactcaacttacgtacgaccatggctgtagacgtgttacaatatcacctcagtcattcttatcacttagacacattagtatgttaagctcatccttatcgaccatggctgtagacgtgttacactcaaatcatcttaacaactaatgtttagtagtcaatacagattatgactctatcaaacgaccatggctgtagacgtgttagcatcatcatcacataatcgtattgacatgcaatctatatacttcaaggaagatgactaacgaccatggctgtagacgtgttacactgctagtaaccttgaatatatagctgaagttctgtttattgacatgtcaacatcaatcgaccatggctgtagacgtgttacactttacctaacatgtcataaacagtaaccgaa

SEQ ID NO: 217 - DNA, 501 bp, synthetic construct
  source 1..501: mol_type = other DNA
gggtgatatacgaccagtcacatacctcccgaccatggctgtagacctgttaatactctcacatactggtctatatcattatgagctgataagaaatgactactattcacaacgaccatggctgtagacctgttaaatccaaattcaagtcattcttatcattcagacacattagtatgttaagcctcacttccccgaccatggctgtagacctgttaaacattccacctcttaacaactaatgtttagtagtcaatacagattatgcaccctatcaacgaccatggctgtagacctgttaaacatacacactcataatcgtattgattctttcactatatacttcaaggccttcttctatcgaccatggctgtagacctgttaacctcactaataccttgaatatatagttcatgccctgtttattgacatgacactccctatcgaccatggctgtagacctgttacttcttctatcacatgtcataaacagttatacaa

SEQ ID NO: 218 - DNA, 501 bp, synthetic construct
  source 1..501: mol_type = other DNA
gggtgatatacgaccagttacttcactctcgaccatggctgtagacttgttacttacttcaactactggtctatatcatcttgagctgataagaaatgactcatactaacttcgaccatggctgtagacttgttaaccttatcattcagtcattcttatcacttagacacattagtatgttaagctcttactaatcgaccatggctgtagacttgttactaacttaccctcttaacaactaatgtttagtagtcaatacagattatgtcattccctatcgaccatggctgtagacttgttaactcttacctcacataatcgtattgacatgcaatctatatacttcaaggtttcatcccttcgaccatggctgtagacttgttaaatcccttcattccttgaatatatagctttagttctgtttattgacatgtccactttaatcgaccatggctgtagacttgttaccaactttcaatcatgtcataaacagtaaccgaa

SEQ ID NO: 219 - DNA, 488 bp, synthetic construct
  source 1..488: mol_type = other DNA
gggtgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagacacattagtatgttaagctcatccttatcgaccatggctcagactgttacactcaaatcatcttaacaactaatgtttagtagtcaatacagattatgactctatcaaacgaccatggctcagactgttagcaacatcatccataatcgtattgacttgcaatctatatacttcaaggaagatcactaacgaccatggctcagactgttacactactaccaaccttgaatatatagccgtagttctgtttattgacatgtcaacatcaatcgaccatggctcagactgttacactttacctaacatgtcataaacagtaacagaa

SEQ ID NO: 220 - DNA, 495 bp, synthetic construct
  source 1..495: mol_type = other DNA
gggtgatatacgaccagtatagactacaacgaccatggctcagacatgttagaaactcaagatactggtctatatcattatgagctgataagaaatgactcaacttacgtacgaccatggctcagacatgttacaatatcacctcagtcattcttatcacttagacacattagtatgttaagctcatccttatcgaccatggctcagacatgttacactcaaatcatcttaacaactaatgtttagtagtcaatacagattatgactctatcaaacgaccatggctcagacatgttagcaacatcatcacataatcgtattgacttgcaatctatatacttcaaggaagatcactaacgaccatggctcagacatgttacactactaccaaccttgaatatatagccgtagttctgtttattgacatgtcaacatcaatcgaccatggctcagacatgttacactttacctaacatgtcataaacagtaacagaa

SEQ ID NO: 221 - DNA, 495 bp, synthetic construct
  source 1..495: mol_type = other DNA
gggtgatatacgaccagtatagactacaacgaccatggctcagacgtgttagaaactcaagatactggtctatatcattatgagctgataagaaatgactcaacttacgtacgaccatggctcagacgtgttacaatatcacctcagtcattcttatcacttagacacattagtatgttaagctcatccttatcgaccatggctcagacgtgttacactcaaatcatcttaacaactaatgtttagtagtcaatacagattatgactctatcaaacgaccatggctcagacgtgttagcaacatcatcacataatcgtattgacttgcaatctatatacttcaaggaagatcactaacgaccatggctcagacgtgttacactactaccaaccttgaatatatagccgtagttctgtttattgacatgtcaacatcaatcgaccatggctcagacgtgttacactttacctaacatgtcataaacagtaacagaa

SEQ ID NO: 222 - DNA, 495 bp, synthetic construct
  source 1..495: mol_type = other DNA
gggtgatatacgaccagtatagactacaacgaccatggctcagacctgttagaaactcaagatactggtctatatcattatgagctgataagaaatgactcaacttacgtacgaccatggctcagacctgttacaatatcacctcagtcattcttatcacttagacacattagtatgttaagctcatccttatcgaccatggctcagacctgttacactcaaatcatcttaacaactaatgtttagtagtcaatacagattatgactctatcaaacgaccatggctcagacctgttagcaacatcatcacataatcgtattgacttgcaatctatatacttcaaggaagatcactaacgaccatggctcagacctgttacactactaccaaccttgaatatatagccgtagttctgtttattgacatgtcaacatcaatcgaccatggctcagacctgttacactttacctaacatgtcataaacagtaacagaa

SEQ ID NO: 223 - DNA, 495 bp, synthetic construct
  source 1..495: mol_type = other DNA
gggtgatatacgaccagtatagactacaacgaccatggctcagacttgttagaaactcaagatactggtctatatcattatgagctgataagaaatgactcaacttacctacgaccatggctcagacttgttacaatatcacctcagtcattcttatcacttagacacattagtatgttaagcttacgcataccgaccatggctcagacttgttacactctaatcctcttaacaactaatgtttagtagtcaatacagattatgactctatcaaacgaccatggctcagacttgttagcaacatcatcacataatcgtattgacttgcaatctatatacttcaaggatcctcacactcgaccatggctcagacttgttacactacatccatccttgaatatatagccgtagttctgtttattgacatgtcaacatcaatcgaccatggctcagacttgttacactttacctaacatgtcataaacagtaacagaa

SEQ ID NO: 224 - DNA, 513 bp, synthetic construct
  source 1..513: mol_type = other DNA
gggtgatatacgaccagtgcgcactacacaacccctatcacgattagcaattaagaaactcaagatactggtctatatcattatgagctgataagaaatgactactttaactacaacccctatcacgattagcaattaagaacacgacatcagtcattcttatcattcagacacattagtatgttaagttaagcatcataacccctatcacgattagcaattaacaacgaaacgatcttaacaactaatgtttagtagtcaatacagattatgacagcatctataacccctatcacgattagcaattaatacaccacgacgcataatcgtattgattctttcactatatacttcaaggaccatcactacaacccctatcacgattagcaattaacactactagcagccttgaatatatagttcatgccctgtttattgacatgtcacatctcacaacccctatcacgattagcaattaacacatcacgcatcatgtcataaacagttatacaa

SEQ ID NO: 225 - DNA, 513 bp, synthetic construct
  source 1..513: mol_type = other DNA
gggtgatatacgaccagttcatactacacaacccctatcacgattagcgattaagaaactcaatatactggtctatatcattatgagctgataagaaatgactacaccgacataaacccctatcacgattagcgattaacctacagagattagtcattcttatcattcagacacattagtatgttaagtgactcatcataacccctatcacgattagcgattaaattcagaacgatcttaacaactaatgtttagtagtcaatacagattatgacagcaactataacccctatcacgattagcgattaatacaccacgaatcataatcgtattgattctttcactatatacttcaaggacatacctatcaacccctatcacgattagcgattaacaccatgaccagccttgaatatatagttcatgccctgtttattgacatgtcacatctcacaacccctatcacgattagcgattaacactccactcatcatgtcataaacagttatacaa

SEQ ID NO: 226 - DNA, 513 bp, synthetic construct
  source 1..513: mol_type = other DNA
gggtgatatacgaccagtgcgcactacacaacccctatcacgattagccattaagaaactcaagatactggtctatatcattatgagctgataagaaatgactactttaactacaacccctatcacgattagccattaagaacacgacatcagtcattcttatcattcagacacattagtatgttaagttaagcatcataacccctatcacgattagccattaacaacgaaacgatcttaacaactaatgtttagtagtcaatacagattatgacagcatctataacccctatcacgattagccattaatacaccacgacgcataatcgtattgattctttcactatatacttcaaggaccatcactacaacccctatcacgattagccattaacactactagcagccttgaatatatagttcatgccctgtttattgacatgtcacatctcacaacccctatcacgattagccattaacacatcacgcatcatgtcataaacagttatacaa

SEQ ID NO: 227 - DNA, 513 bp, synthetic construct
  source 1..513: mol_type = other DNA
gggtgatatacgaccagtgcgcactacacaacccctatcacgattagctattaagaaactcaagatactggtctatatcattatgagctgataagaaatgactactttaactacaacccctatcacgattagctattaagaacacgacatcagtcattcttatcattcagacacattagtatgttaagttaccgatcataacccctatcacgattagctattaacttacaaacgatcttaacaactaatgtttagtagtcaatacagattatgacatcacgcataacccctatcacgattagctattaatacaccacgacgcataatcgtattgattctttcactatatacttcaaggactctatctacaacccctatcacgattagctattaacacttcacgcctccttgaatatatagttcatgccctgtttattgacatgtcacatctcacaacccctatcacgattagctattaacacatcacgcatcatgtcataaacagttatacaa

SEQ ID NO: 228 - DNA, 501 bp, synthetic construct
  source 1..501: mol_type = other DNA
gggtgatatacgaccagtgcgcactacacaacccctatcacgtaagcattaagaaactcaagatactggtctatatcattatgagctgataagaaatgactatccgaactacaacccctatcacgtaagcattaatcatccaacatgagtcattcttatcattcagacacattagtatgttaagtgactcatcataacccctatcacgtaagcattaactactcaacgatcttaacaactaatgtttagtagtcaatacagattatgacagcatctataacccctatcacgtaagcattaatacaccacgacgcataatcgtattgattctttcactatatacttcaaggaccatcactacaacccctatcacgtaagcattaacactactagcagccttgaatatatagttcatgccctgtttattgacatgtcacatctcacaacccctatcacgtaagcattaacacatcacgcatcatgtcataaacagttatacaa

SEQ ID NO: 229 - DNA, 507 bp, synthetic construct
  source 1..507: mol_type = other DNA
gggtgatatacgaccagtgcgcactacacaacccctatcacgtaagcaattaagaaactcaagatactggtctatatcattatgagctgataagaaatgactatccgaactacaacccctatcacgtaagcaattaatcatccaacatgagtcattcttatcattcagacacattagtatgttaagtgactcatcataacccctatcacgtaagcaattaactactcaacgatcttaacaactaatgtttagtagtcaatacagattatgacagcatctataacccctatcacgtaagcaattaatacaccacgacgcataatcgtattgattctttcactatatacttcaaggaccatcactacaacccctatcacgtaagcaattaacactactagcagccttgaatatatagttcatgccctgtttattgacatgtcacatctcacaacccctatcacgtaagcaattaacacatcacgcatcatgtcataaacagttatacaa

SEQ ID NO: 230 - DNA, 507 bp, synthetic construct
  source 1..507: mol_type = other DNA
gggtgatatacgaccagttcagactacacaacccctatcacgtaagcgattaagaaactcaatatactggtctatatcattatgagctgataagaaatgactacaccgacataaacccctatcacgtaagcgattaacctacagagattagtcattcttatcattcagacacattagtatgttaagtgactcagcataacccctatcacgtaagcgattaaattcagaacaatcttaacaactaatgtttagtagtcaatacagattatgacagcaactataacccctatcacgtaagcgattaatacaccacgaatcataatcgtattgattctttcactatatacttcaaggacatacctatcaacccctatcacgtaagcgattaacaccatcaccagccttgaatatatagttcatgccctgtttattgacatgtcacatctcacaacccctatcacgtaagcgattaacactccactcatcatgtcataaacagttatacaa

SEQ ID NO: 231 - DNA, 507 bp, synthetic construct
  source 1..507: mol_type = other DNA
gggtgatatacgaccagtgcgcactacacaacccctatcacgtaagccattaagaaactcaagatactggtctatatcattatgagctgataagaaatgactatccgaactacaacccctatcacgtaagccattaatcatccaacatgagtcattcttatcattcagacacattagtatgttaagtgactcatcataacccctatcacgtaagccattaactactcaacgatcttaacaactaatgtttagtagtcaatacagattatgacagcatctataacccctatcacgtaagccattaatacaccacgacgcataatcgtattgattctttcactatatacttcaaggaccatcactacaacccctatcacgtaagccattaacactactagcagccttgaatatatagttcatgccctgtttattgacatgtcacatctcacaacccctatcacgtaagccattaacacatcacgcatcatgtcataaacagttatacaa

SEQ ID NO: 232 - DNA, 507 bp, synthetic construct
  source 1..507: mol_type = other DNA
gggtgatatacgaccagtgcgcactacacaacccctatcacgtaagctattaagaaactcaagatactggtctatatcattatgagctgataagaaatgactatccgaactacaacccctatcacgtaagctattaatcatccaacatgagtcattcttatcattcagacacattagtatgttaagtgactcatcataacccctatcacgtaagctattaactactcaacgatcttaacaactaatgtttagtagtcaatacagattatgacaccatctataacccctatcacgtaagctattaatacaccacgacgcataatcgtattgattctttcactatatacttcaaggaccatcacaacaacccctatcacgtaagctattaacacgaccacgagccttgaatatatagttcatgccctgtttattgacatgtcacatctcacaacccctatcacgtaagctattaacacatcacgcatcatgtcataaacagttatacaa

SEQ ID NO: 233 - DNA, 501 bp, synthetic construct
  source 1..501: mol_type = other DNA
gggtgatatacgaccagtccgcactacacctatctgcactagatgcaccttataaactcagaatactggtctatatcattatgagctgataagaaatgactagctcaagtccctatctgcactagatgcaccttaataagtcctccgagtcattcttatcattcagacacattagtatgttaagtcactcacgatctatctgcactagatgcaccttactactcaactatcttaacaactaatgtttagtagtcaatacagattatgacagcatagatctatctgcactagatgcaccttatacaccacgacgcataatcgtattgattctttcactatatacttcaaggaccatcactacctatctgcactagatgcaccttacactactaccagccttgaatatatagttcatgccctgtttattgacatgtcacatctcacctatctgcactagatgcaccttacacatcacgcatcatgtcataaacagttatacaa

SEQ ID NO: 234 - DNA, 507 bp, synthetic construct
  source 1..507: mol_type = other DNA
gggtgatatacgaccagtccgcactacacctatctgcactagatgcaaccttataaactcagaatactggtctatatcattatgagctgataagaaatgactagctcaagtccctatctgcactagatgcaaccttaataagtcctccgagtcattcttatcattcagacacattagtatgttaagtcactcacgatctatctgcactagatgcaaccttactactcaactatcttaacaactaatgtttagtagtcaatacagattatgacagcatagatctatctgcactagatgcaaccttatacaccacgacgcataatcgtattgattctttcactatatacttcaaggaccatcactacctatctgcactagatgcaaccttacactactaccagccttgaatatatagttcatgccctgtttattgacatgtcacatctcacctatctgcactagatgcaaccttacacatcacgcatcatgtcataaacagttatacaa

SEQ ID NO: 235 - DNA, 507 bp, synthetic construct
  source 1..507: mol_type = other DNA
gggtgatatacgaccagtccgcactacacctatctgcactagatgcagccttataaactcagaatactggtctatatcattatgagctgataagaaatgactagctcaagtccctatctgcactagatgcagccttaataagtcctccgagtcattcttatcattcagacacattagtatgttaagtcactcacgatctatctgcactagatgcagccttactactcaactatcttaacaactaatgtttagtagtcaatacagattatgacagcatagatctatctgcactagatgcagccttatacaccacgacgcataatcgtattgattctttcactatatacttcaaggaccatcactacctatctgcactagatgcagccttacactactaccagccttgaatatatagttcatgccctgtttattgacatgtcacatctcacctatctgcactagatgcagccttacacatcacgcatcatgtcataaacagttatacaa

SEQ ID NO: 236 - DNA, 507 bp, synthetic construct
  source 1..507: mol_type = other DNA
gggtgatatacgaccagtccgcactacacctatctgcactagatgcacccttataaactcagaatactggtctatatcattatgagctgataagaaatgactagctcaagtccctatctgcactagatgcacccttaataagtcctccgagtcattcttatcattcagacacattagtatgttaagtcactcacgatctatctgcactagatgcacccttactactcaactatcttaacaactaatgtttagtagtcaatacagattatgacagcatagatctatctgcactagatgcacccttatacaccacgacgcataatcgtattgattctttcactatatacttcaaggaccatcactacctatctgcactagatgcacccttacactactaccagccttgaatatatagttcatgccctgtttattgacatgtcacatctcacctatctgcactagatgcacccttacacatcacgcatcatgtcataaacagttatacaa

SEQ ID NO: 237 - DNA, 507 bp, synthetic construct
  source 1..507: mol_type = other DNA
gggtgatatacgaccagtccgcactacacctatctgcactagatgcatccttataaactcagaatactggtctatatcattatgagctgataagaaatgactagctcaagtccctatctgcactagatgcatccttaataagtcctccgagtcattcttatcattcagacacattagtatgttaagtcactcactatctatctgcactagatgcatccttactactcaactatcttaacaactaatgtttagtagtcaatacagattatgacagcatagacctatctgcactagatgcatccttatacaccacgacgcataatcgtattgattctttcactatatacttcaaggaccatcactacctatctgcactagatgcatccttacactactaccagccttgaatatatagttcatgccctgtttattgacatgtcacatctcacctatctgcactagatgcatccttacacatcacgcatcatgtcataaacagttatacaa

SEQ ID NO: 238 - DNA, 495 bp, synthetic construct
  source 1..495: mol_type = other DNA
gggtgatatacgaccagtccgcactacacctatctgcactactgcaccttataaactcagaatactggtctatatcattatgagctgataagaaatgacttcttcaagtccctatctgcactactgcaccttaataagtcctcctagtcattcttatcattcagacacattagtatgttaagtgactcatcatctatctgcactactgcaccttactactcaacgatcttaacaactaatgtttagtagtcaatacagattatgacagcatctatctatctgcactactgcaccttatacaccacgacgcataatcgtattgattctttcactatatacttcaaggaccatcactacctatctgcactactgcaccttacactactagcagccttgaatatatagttcatgccctgtttattgacatgtcacatctcacctatctgcactactgcaccttacacatcacgcatcatgtcataaacagttatacaa

SEQ ID NO: 239 - DNA, 501 bp, synthetic construct
  source 1..501: mol_type = other DNA
gggtgatatacgaccagtccgcactacacctatctgcactactgcaaccttataaactcagaatactggtctatatcattatgagctgataagaaatgacttcttcaagtccctatctgcactactgcaaccttaataagtcctcctagtcattcttatcattcagacacattagtatgttaagtcactcatcatctatctgcactactgcaaccttactactcaactgtcttaacaactaatgtttagtagtcaatacagattatgacagcatagatctatctgcactactgcaaccttatacaccacgacgcataatcgtattgattctttcactatatacttcaaggaccatcactacctatctgcactactgcaaccttacactactaccagccttgaatatatagttcatgccctgtttattgacatgtcacatctcacctatctgcactactgcaaccttacacatcacgcatcatgtcataaacagttatacaa

SEQ ID NO: 240 - DNA, 501 bp, synthetic construct
  source 1..501: mol_type = other DNA
gggtgatatacgaccagtccgcactacacctatctgcactactgcagccttataaactcagaatactggtctatatcattatgagctgataagaaatgacttcttcaagtccctatctgcactactgcagccttaataagtcctcctagtcattcttatcattcagacacattagtatgttaagtcactcatcatctatctgcactactgcagccttactactcaactgtcttaacaactaatgtttagtagtcaatacagattatgacagcatagatctatctgcactactgcagccttatacaccacgacgcataatcgtattgattctttcactatatacttcaaggaccatcactacctatctgcactactgcagccttacactactaccagccttgaatatatagttcatgccctgtttattgacatgtcacatctcacctatctgcactactgcagccttacacatcacgcatcatgtcataaacagttatacaa

SEQ ID NO: 241 - DNA, 501 bp, synthetic construct
  source 1..501: mol_type = other DNA
gggtgatatacgaccagtccgcactacacctatctgcactactgcacccttataaactcagaatactggtctatatcattatgagctgataagaaatgacttcttcaagtccctatctgcactactgcacccttaataagtcctcctagtcattcttatcattcagacacattagtatgttaagtcactcatcatctatctgcactactgcacccttactactcaactgtcttaacaactaatgtttagtagtcaatacagattatgacagcatagatctatctgcactactgcacccttatacaccacgacgcataatcgtattgattctttcactatatacttcaaggaccatcactacctatctgcactactgcacccttacactactaccagccttgaatatatagttcatgccctgtttattgacatgtcacatctcacctatctgcactactgcacccttacacatcacgcatcatgtcataaacagttatacaa

SEQ ID NO: 242 - DNA, 501 bp, synthetic construct
  source 1..501: mol_type = other DNA
gggtgatatacgaccagtccgcactacacctatctgcactactgcatccttataaactcagaatactggtctatatcattatgagctgataagaaatgacttcttcaagtccctatctgcactactgcatccttaataagtcctcctagtcattcttatcattcagacacattagtatgttaagtcactcatcatctatctgcactactgcatccttactactcaactgtcttaacaactaatgtttagtagtcaatacagattatgacagcatagatctatctgcactactgcatccttatacaccacgacgcataatcgtattgattctttcactatatacttcaaggaccatcactacctatctgcactactgcatccttacactactaccagccttgaatatatagttcatgccctgtttattgacatgtcacatctcacctatctgcactactgcatccttacacatcacgcatcatgtcataaacagttatacaa

SEQ ID NO: 243 - DNA, 501 bp, synthetic construct
  source 1..501: mol_type = other DNA
gggtgatatacgaccagtcctcactactactacctgcactgtaagcactttgacttcgccgattactggtctatatcattatgagctgataagaaatgactatcctgactacctacctgcactgtaagcactttgacatcctacctgagtcattcttatcattcagacacattagtatgttaagtcaagccacatctacctgcactgtaagcactttgctaagcgacactcttaacaactaatgtttagtagtcaatacagattatgacttccgatctctacctgcactgtaagcactttgtcctattacgtccataatcgtattgattctttcactatatacttcaaggaccatcacgaactacctgcactgtaagcactttgacctactaccatccttgaatatatagttcatgccctgtttattgacatgtcactccatacctacctgcactgtaagcactttgaccattcctcttcatgtcataaacagttatacaa

SEQ ID NO: 244 - DNA, 507 bp, synthetic construct
  source 1..507: mol_type = other DNA
gggtgatatacgaccagtcctcactactactacctgcactgtaagcaactttgacttcgccgattactggtctatatcattatgagctgataagaaatgactatcctgactacctacctgcactgtaagcaactttgacatcctacctgagtcattcttatcattcagacacattagtatgttaagtcaagccacatctacctgcactgtaagcaactttgctaagcgacactcttaacaactaatgtttagtagtcaatacagattatgacttccgatctctacctgcactgtaagcaactttgtcctattacgtccataatcgtattgattctttcactatatacttcaaggaccatcacgaactacctgcactgtaagcaactttgacctactaccatccttgaatatatagttcatgccctgtttattgacatgtcactccatacctacctgcactgtaagcaactttgaccattcctcttcatgtcataaacagttatacaa

SEQ ID NO: 245 - DNA, 507 bp, synthetic construct
  source 1..507: mol_type = other DNA
gggtgatatacgaccagtcctcactactactacctgcactgtaagcagctttgacttcgccgattactggtctatatcattatgagctgataagaaatgactatcctgactacctacctgcactgtaagcagctttgacatcctacctgagtcattcttatcattcagacacattagtatgttaagtcaagccacatctacctgcactgtaagcagctttgctaagcgacactcttaacaactaatgtttagtagtcaatacagattatgacttccgatctctacctgcactgtaagcagctttgtcctattacgtccataatcgtattgattctttcactatatacttcaaggaccatcacgaactacctgcactgtaagcagctttgacctactaccatccttgaatatatagttcatgccctgtttattgacatgtcactccatacctacctgcactgtaagcagctttgaccattcctcttcatgtcataaacagttatacaa

SEQ ID NO: 246 - DNA, 507 bp, synthetic construct
  source 1..507: mol_type = other DNA
gggtgatatacgaccagtcctcactactactacctgcactgtaagcacctttgacttcgccgattactggtctatatcattatgagctgataagaaatgactatcctgactacctacctgcactgtaagcacctttgacatcctacctgagtcattcttatcattcagacacattagtatgttaagtcaagccacatctacctgcactgtaagcacctttgctaagcgacactcttaacaactaatgtttagtagtcaatacagattatgacttccgatctctacctgcactgtaagcacctttgtcctattacgtccataatcgtattgattctttcactatatacttcaaggaccatcacgaactacctgcactgtaagcacctttgacctactaccatccttgaatatatagttcatgccctgtttattgacatgtcactccatacctacctgcactgtaagcacctttgaccattcctcttcatgtcataaacagttatacaa

SEQ ID NO: 247 - DNA, 507 bp, synthetic construct
  source 1..507: mol_type = other DNA
gggtgatatacgaccagtcctcactactactacctgcactgtaagcatctttgacttcgccgattactggtctatatcattatgagctgataagaaatgactatcctgactacctacctgcactgtaagcatctttgacatcctacctgagtcattcttatcattcagacacattagtatgttaagtcatgacacacctacctgcactgtaagcatctttgacatccgacactcttaacaactaatgtttagtagtcaatacagattatgacttccgatctctacctgcactgtaagcatctttgtcctattacgtccataatcgtattgattctttcactatatacttcaaggaccatcacgaactacctgcactgtaagcatctttgacctactaccatccttgaatatatagttcatgccctgtttattgacatgtcactccatacctacctgcactgtaagcatctttgaccattcctcttcatgtcataaacagttatacaa

SEQ ID NO: 248 - DNA, 495 bp, synthetic construct
  source 1..495: mol_type = other DNA
gggtgatatacgaccagtccgcactacacctacctgcactgatgcactttgtaaactcctgatactggtctatatcattatgagctgataagaaatgactatccgaactacctacctgcactgatgcactttgccatccaacctgagtcattcttatcattcagacacattagtatgttaagtgactcctcatctacctgcactgatgcactttgctactcaactatcttaacaactaatgtttagtagtcaatacagattatgacatcatatatctacctgcactgatgcactttgcccacctcgacgcataatcgtattgattctttcactatatacttcaaggaccatcactatctacctgcactgatgcactttgtcctactaccagccttgaatatatagttcatgccctgtttattgacatgtcacatctcacctacctgcactgatgcactttgcataacactcatcatgtcataaacagttatacaa

SEQ ID NO: 249 - DNA, 501 bp, synthetic construct
  source 1..501: mol_type = other DNA
gggtgatatacgaccagtccatacgacacctacctgcactgatgcaactttgctaactgatcatactggtctatatcattatgagctgataagaaatgactatccgaactacctacctgcactgatgcaactttgacatgctgactgagtcattcttatcattcagacacattagtatgttaagtgactccacatctacctgcactgatgcaactttgctacgcctctatcttaacaactaatgtttagtagtcaatacagattatgactttcacgccctacctgcactgatgcaactttgactactttaccgcataatcgtattgattctttcactatatacttcaaggagtctcacgctctacctgcactgatgcaactttgtactactaccagccttgaatatatagttcatgccctgtttattgacatgcgacaccactcctacctgcactgatgcaactttgctgaacactgatcatgtcataaacagttatacaa

SEQ ID NO: 250 - DNA, 501 bp, synthetic construct
  source 1..501: mol_type = other DNA
gggtgatatacgaccagtccatacgacacctacctgcactgatgcagctttgctaactgatcatactggtctatatcattatgagctgataagaaatgactatccgaactacctacctgcactgatgcagctttgacatgctgactgagtcattcttatcattcagacacattagtatgttaagtgactccacatctacctgcactgatgcagctttgctacgcctctatcttaacaactaatgtttagtagtcaatacagattatgactttcacgccctacctgcactgatgcagctttgactactttaccgcataatcgtattgattctttcactatatacttcaaggagtctcacgctctacctgcactgatgcagctttgtactactaccagccttgaatatatagttcatgccctgtttattgacatgcgacaccactcctacctgcactgatgcagctttgctgaacactgatcatgtcataaacagttatacaa

SEQ ID NO: 251 - DNA, 501 bp, synthetic construct
  source 1..501: mol_type = other DNA
gggtgatatacgaccagtccatacgacacctacctgcactgatgcacctttgctaactgatcatactggtctatatcattatgagctgataagaaatgactatccgaactacctacctgcactgatgcacctttgacatgctgactgagtcattcttatcattcagacacattagtatgttaagtgactccacatctacctgcactgatgcacctttgctacgcctctatcttaacaactaatgtttagtagtcaatacagattatgactttcacgccctacctgcactgatgcacctttgactactttaccgcataatcgtattgattctttcactatatacttcaaggagtctcacgctctacctgcactgatgcacctttgtactactaccagccttgaatatatagttcatgccctgtttattgacatgcgacaccactcctacctgcactgatgcacctttgctgaacactgatcatgtcataaacagttatacaa

SEQ ID NO: 252 - DNA, 501 bp, synthetic construct
  source 1..501: mol_type = other DNA
gggtgatatacgaccagtcctcactactactacctgcactgatgcatctttgacttcgccgattactggtctatatcattatgagctgataagaaatgactatcctgactacctacctgcactgatgcatctttgacctgctacctgagtcattcttatcattcagacacattagtatgttaagtcaagccacatctacctgcactgatgcatctttgctaagcgacactcttaacaactaatgtttagtagtcaatacagattatgacttccgatctctacctgcactgatgcatctttgtcctattacgtccataatcgtattgattctttcactatatacttcaaggaccttcacgaactacctgcactgatgcatctttgacctactaccatccttgaatatatagttcatgccctgtttattgacatgtcactccatacctacctgcactgatgcatctttgaccattcctcttcatgtcataaacagttatacaa

SEQ ID NO: 253 - RNA, 492 nt, synthetic construct
  source 1..492: mol_type = other RNA
tgatatacgaccagttcttattctcccgaccatggctgtagactgttaaatacttcaaatactggtctatatcattatgagctgataagaaatgacttcaaccaaatccgaccatggctgtagactgttaacaatatcccttagtcattcttatcattcagacacattagtatgttaagtatatcaaattcgaccatggctgtagactgttaatattctctcctcttaacaactaatgtttagtagtcaatacagattatgtacacataattcgaccatggctgtagactgttaataatcctatttcataatcgtattgattctttcactatatacttcaaggtattctcttctcgaccatggctgtagactgttaacacaacacactccttgaatatatagttcatgccctgtttattgacatgtactcatatttcgaccatggctgtagactgttaatttatcctattcatgtcataaacagttatacaa

SEQ ID NO: 254 - RNA, 498 nt, synthetic construct
  source 1..498: mol_type = other RNA
tgatatacgaccagtatagactacaacgaccatggctgtagacatgttagaaactcaagatactggtctatatcattatgagctgataagaaatgactcaacttacgtacgaccatggctgtagacatgttacaatatcacctcagtcattcttatcacttagacacattagtatgttaagctcatccttatcgaccatggctgtagacatgttacactcaaatcatcttaacaactaatgtttagtagtcaatacagattatgactctatcaaacgaccatggctgtagacatgttagcaacatcatcacataatcgtattgacatgcaatctatatacttcaaggaagatgactaacgaccatggctgtagacatgttacactgctagtaaccttgaatatatagctgtagttctgtttattgacatgtcaacatcaatcgaccatggctgtagacatgttacactttacctaacatgtcataaacagtaactgaa

SEQ ID NO: 255 - RNA, 498 nt, synthetic construct
  source 1..498: mol_type = other RNA
tgatatacgaccagtatagactacaacgaccatggctgtagacgtgttagaaactcaagatactggtctatatcattatgagctgataagaaatgactcaacttacgtacgaccatggctgtagacgtgttacaatatcacctcagtcattcttatcacttagacacattagtatgttaagctcatccttatcgaccatggctgtagacgtgttacactcaaatcatcttaacaactaatgtttagtagtcaatacagattatgactctatcaaacgaccatggctgtagacgtgttagcatcatcatcacataatcgtattgacatgcaatctatatacttcaaggaagatgactaacgaccatggctgtagacgtgttacactgctagtaaccttgaatatatagctgaagttctgtttattgacatgtcaacatcaatcgaccatggctgtagacgtgttacactttacctaacatgtcataaacagtaaccgaa

SEQ ID NO: 256 - RNA, 498 nt, synthetic construct
  source 1..498: mol_type = other RNA
tgatatacgaccagtcacatacctcccgaccatggctgtagacctgttaatactctcacatactggtctatatcattatgagctgataagaaatgactactattcacaacgaccatggctgtagacctgttaaatccaaattcaagtcattcttatcattcagacacattagtatgttaagcctcacttccccgaccatggctgtagacctgttaaacattccacctcttaacaactaatgtttagtagtcaatacagattatgcaccctatcaacgaccatggctgtagacctgttaaacatacacactcataatcgtattgattctttcactatatacttcaaggccttcttctatcgaccatggctgtagacctgttaacctcactaataccttgaatatatagttcatgccctgtttattgacatgacactccctatcgaccatggctgtagacctgttacttcttctatcacatgtcataaacagttatacaa

SEQ ID NO: 257 - RNA, 498 nt, synthetic construct
  source 1..498: mol_type = other RNA
tgatatacgaccagttacttcactctcgaccatggctgtagacttgttacttacttcaactactggtctatatcatcttgagctgataagaaatgactcatactaacttcgaccatggctgtagacttgttaaccttatcattcagtcattcttatcacttagacacattagtatgttaagctcttactaatcgaccatggctgtagacttgttactaacttaccctcttaacaactaatgtttagtagtcaatacagattatgtcattccctatcgaccatggctgtagacttgttaactcttacctcacataatcgtattgacatgcaatctatatacttcaaggtttcatcccttcgaccatggctgtagacttgttaaatcccttcattccttgaatatatagctttagttctgtttattgacatgtccactttaatcgaccatggctgtagacttgttaccaactttcaatcatgtcataaacagtaaccgaa

SEQ ID NO: 258 - RNA, 486 nt, synthetic construct
  source 1..486: mol_type = other RNA
tgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagacacattagtatgttaagctcatccttatcgaccatggctcagactgttacactcaaatcatcttaacaactaatgtttagtagtcaatacagattatgactctatcaaacgaccatggctcagactgttagcaacatcatcacataatcgtattgacttgcaatctatatacttcaaggaagatcactaacgaccatggctcagactgttacactactaccaaccttgaatatatagccgtagttctgtttattgacatgtcaacatcaatcgaccatggctcagactgttacactttacctaacatgtcataaacagtaacagaa

SEQ ID NO: 259 - RNA, 492 nt, synthetic construct
  source 1..492: mol_type = other RNA
tgatatacgaccagtatagactacaacgaccatggctcagacatgttagaaactcaagatactggtctatatcattatgagctgataagaaatgactcaacttacgtacgaccatggctcagacatgttacaatatcacctcagtcattcttatcacttagacacattagtatgttaagctcatccttatcgaccatggctcagacatgttacactcaaatcatcttaacaactaatgtttagtagtcaatacagattatgactctatcaaacgaccatggctcagacatgttagcaacatcatcacataatcgtattgacttgcaatctatatacttcaaggaagatcactaacgaccatggctcagacatgttacactactaccaaccttgaatatatagccgtagttctgtttattgacatgtcaacatcaatcgaccatggctcagacatgttacactttacctaacatgtcataaacagtaacagaa

SEQ ID NO: 260 - RNA, 492 nt, synthetic construct
  source 1..492: mol_type = other RNA
tgatatacgaccagtatagactacaacgaccatggctcagacgtgttagaaactcaagatactggtctatatcattatgagctgataagaaatgactcaacttacgtacgaccatggctcagacgtgttacaatatcacctcagtcattcttatcacttagacacattagtatgttaagctcatccttatcgaccatggctcagacgtgttacactcaaatcatcttaacaactaatgtttagtagtcaatacagattatgactctatcaaacgaccatggctcagacgtgttagcaacatcatcacataatcgtattgacttgcaatctatatacttcaaggaagatcactaacgaccatggctcagacgtgttacactactaccaaccttgaatatatagccgtagttctgtttattgacatgtcaacatcaatcgaccatggctcagacgtgttacactttacctaacatgtcataaacagtaacagaa

SEQ ID NO: 261 - RNA, 492 nt, synthetic construct
  source 1..492: mol_type = other RNA
tgatatacgaccagtatagactacaacgaccatggctcagacctgttagaaactcaagatactggtctatatcattatgagctgataagaaatgactcaacttacgtacgaccatggctcagacctgttacaatatcacctcagtcattcttatcacttagacacattagtatgttaagctcatccttatcgaccatggctcagacctgttacactcaaatcatcttaacaactaatgtttagtagtcaatacagattatgactctatcaaacgaccatggctcagacctgttagcaacatcatcacataatcgtattgacttgcaatctatatacttcaaggaagatcactaacgaccatggctcagacctgttacactactaccaaccttgaatatatagccgtagttctgtttattgacatgtcaacatcaatcgaccatggctcagacctgttacactttacctaacatgtcataaacagtaacagaa

SEQ ID NO: 262 - RNA, 492 nt, synthetic construct
  source 1..492: mol_type = other RNA
tgatatacgaccagtatagactacaacgaccatggctcagacttgttagaaactcaagatactggtctatatcattatgagctgataagaaatgactcaacttacctacgaccatggctcagacttgttacaatatcacctcagtcattcttatcacttagacacattagtatgttaagcttacgcataccgaccatggctcagacttgttacactctaatcctcttaacaactaatgtttagtagtcaatacagattatgactctatcaaacgaccatggctcagacttgttagcaacatcatcacataatcgtattgacttgcaatctatatacttcaaggatcctcacactcgaccatggctcagacttgttacactacatccatccttgaatatatagccgtagttctgtttattgacatgtcaacatcaatcgaccatggctcagacttgttacactttacctaacatgtcataaacagtaacagaa

SEQ ID NO: 263 - RNA, 510 nt, synthetic construct
  source 1..510: mol_type = other RNA
tgatatacgaccagtgcgcactacacaacccctatcacgattagcaattaagaaactcaagatactggtctatatcattatgagctgataagaaatgactactttaactacaacccctatcacgattagcaattaagaacacgacatcagtcattcttatcattcagacacattagtatgttaagttaagcatcataacccctatcacgattagcaattaacaacgaaacgatcttaacaactaatgtttagtagtcaatacagattatgacagcatctataacccctatcacgattagcaattaatacaccacgacgcataatcgtattgattctttcactatatacttcaaggaccatcactacaacccctatcacgattagcaattaacactactagcagccttgaatatatagttcatgccctgtttattgacatgtcacatctcacaacccctatcacgattagcaattaacacatcacgcatcatgtcataaacagttatacaa

SEQ ID NO: 264 - RNA, 510 nt, synthetic construct
  source 1..510: mol_type = other RNA
tgatatacgaccagttcatactacacaacccctatcacgattagcgattaagaaactcaatatactggtctatatcattatgagctgataagaaatgactacaccgacataaacccctatcacgattagcgattaacctacagagattagtcattcttatcattcagacacattagtatgttaagtgactcatcataacccctatcacgattagcgattaaattcagaacgatcttaacaactaatgtttagtagtcaatacagattatgacagcaactataacccctatcacgattagcgattaatacaccacgaatcataatcgtattgattctttcactatatacttcaaggacatacctatcaacccctatcacgattagcgattaacaccatgaccagccttgaatatatagttcatgccctgtttattgacatgtcacatctcacaacccctatcacgattagcgattaacactccactcatcatgtcataaacagttatacaa

SEQ ID NO: 265 - RNA, 510 nt, synthetic construct
  source 1..510: mol_type = other RNA
tgatatacgaccagtgcgcactacacaacccctatcacgattagccattaagaaactcaagatactggtctatatcattatgagctgataagaaatgactactttaactacaacccctatcacgattagccattaagaacacgacatcagtcattcttatcattcagacacattagtatgttaagttaagcatcataacccctatcacgattagccattaacaacgaaacgatcttaacaactaatgtttagtagtcaatacagattatgacagcatctataacccctatcacgattagccattaatacaccacgacgcataatcgtattgattctttcactatatacttcaaggaccatcactacaacccctatcacgattagccattaacactactagcagccttgaatatatagttcatgccctgtttattgacatgtcacatctcacaacccctatcacgattagccattaacacatcacgcatcatgtcataaacagttatacaa

SEQ ID NO: 266 - RNA, 510 nt, synthetic construct
  source 1..510: mol_type = other RNA
tgatatacgaccagtgcgcactacacaacccctatcacgattagctattaagaaactcaagatactggtctatatcattatgagctgataagaaatgactactttaactacaacccctatcacgattagctattaagaacacgacatcagtcattcttatcattcagacacattagtatgttaagttaccgatcataacccctatcacgattagctattaacttacaaacgatcttaacaactaatgtttagtagtcaatacagattatgacatcacgcataacccctatcacgattagctattaatacaccacgacgcataatcgtattgattctttcactatatacttcaaggactctatctacaacccctatcacgattagctattaacacttcacgcctccttgaatatatagttcatgccctgtttattgacatgtcacatctcacaacccctatcacgattagctattaacacatcacgcatcatgtcataaacagttatacaa

SEQ ID NO: 267 - RNA, 498 nt, synthetic construct
  source 1..498: mol_type = other RNA
tgatatacgaccagtgcgcactacacaacccctatcacgtaagcattaagaaactcaagatactggtctatatcattatgagctgataagaaatgactatccgaactacaacccctatcacgtaagcattaatcatccaacatgagtcattcttatcattcagacacattagtatgttaagtgactcatcataacccctatcacgtaagcattaactactcaacgatcttaacaactaatgtttagtagtcaatacagattatgacagcatctataacccctatcacgtaagcattaatacaccacgacgcataatcgtattgattctttcactatatacttcaaggaccatcactacaacccctatcacgtaagcattaacactactagcagccttgaatatatagttcatgccctgtttattgacatgtcacatctcacaacccctatcacgtaagcattaacacatcacgcatcatgtcataaacagttatacaa

SEQ ID NO: 268 - RNA, 504 nt, synthetic construct
  source 1..504: mol_type = other RNA
tgatatacgaccagtgcgcactacacaacccctatcacgtaagcaattaagaaactcaagatactggtctatatcattatgagctgataagaaatgactatccgaactacaacccctatcacgtaagcaattaatcatccaacatgagtcattcttatcattcagacacattagtatgttaagtgactcatcataacccctatcacgtaagcaattaactactcaacgatcttaacaactaatgtttagtagtcaatacagattatgacagcatctataacccctatcacgtaagcaattaatacaccacgacgcataatcgtattgattctttcactatatacttcaaggaccatcactacaacccctatcacgtaagcaattaacactactagcagccttgaatatatagttcatgccctgtttattgacatgtcacatctcacaacccctatcacgtaagcaattaacacatcacgcatcatgtcataaacagttatacaa

SEQ ID NO: 269 - RNA, 504 nt, synthetic construct
  source 1..504: mol_type = other RNA
tgatatacgaccagttcagactacacaacccctatcacgtaagcgattaagaaactcaatatactggtctatatcattatgagctgataagaaatgactacaccgacataaacccctatcacgtaagcgattaacctacagagattagtcattcttatcattcagacacattagtatgttaagtgactcagcataacccctatcacgtaagcgattaaattcagaacaatcttaacaactaatgtttagtagtcaatacagattatgacagcaactataacccctatcacgtaagcgattaatacaccacgaatcataatcgtattgattctttcactatatacttcaaggacatacctatcaacccctatcacgtaagcgattaacaccatcaccagccttgaatatatagttcatgccctgtttattgacatgtcacatctcacaacccctatcacgtaagcgattaacactccactcatcatgtcataaacagttatacaa

SEQ ID NO: 270 - RNA, 504 nt, synthetic construct
  source 1..504: mol_type = other RNA
tgatatacgaccagtgcgcactacacaacccctatcacgtaagccattaagaaactcaagatactggtctatatcattatgagctgataagaaatgactatccgaactacaacccctatcacgtaagccattaatcatccaacatgagtcattcttatcattcagacacattagtatgttaagtgactcatcataacccctatcacgtaagccattaactactcaacgatcttaacaactaatgtttagtagtcaatacagattatgacagcatctataacccctatcacgtaagccattaatacaccacgacgcataatcgtattgattctttcactatatacttcaaggaccatcactacaacccctatcacgtaagccattaacactactagcagccttgaatatatagttcatgccctgtttattgacatgtcacatctcacaacccctatcacgtaagccattaacacatcacgcatcatgtcataaacagttatacaa

SEQ ID NO: 271 - RNA, 504 nt, synthetic construct
  source 1..504: mol_type = other RNA
tgatatacgaccagtgcgcactacacaacccctatcacgtaagctattaagaaactcaagatactggtctatatcattatgagctgataagaaatgactatccgaactacaacccctatcacgtaagctattaatcatccaacatgagtcattcttatcattcagacacattagtatgttaagtgactcatcataacccctatcacgtaagctattaactactcaacgatcttaacaactaatgtttagtagtcaatacagattatgacaccatctataacccctatcacgtaagctattaatacaccacgacgcataatcgtattgattctttcactatatacttcaaggaccatcacaacaacccctatcacgtaagctattaacacgaccacgagccttgaatatatagttcatgccctgtttattgacatgtcacatctcacaacccctatcacgtaagctattaacacatcacgcatcatgtcataaacagttatacaa

SEQ ID NO: 272 - RNA, 498 nt, synthetic construct
  source 1..498: mol_type = other RNA
tgatatacgaccagtccgcactacacctatctgcactagatgcaccttataaactcagaatactggtctatatcattatgagctgataagaaatgactagctcaagtccctatctgcactagatgcaccttaataagtcctccgagtcattcttatcattcagacacattagtatgttaagtcactcacgatctatctgcactagatgcaccttactactcaactatcttaacaactaatgtttagtagtcaatacagattatgacagcatagatctatctgcactagatgcaccttatacaccacgacgcataatcgtattgattctttcactatatacttcaaggaccatcactacctatctgcactagatgcaccttacactactaccagccttgaatatatagttcatgccctgtttattgacatgtcacatctcacctatctgcactagatgcaccttacacatcacgcatcatgtcataaacagttatacaa

SEQ ID NO: 273 - RNA, 504 nt, synthetic construct
  source 1..504: mol_type = other RNA
tgatatacgaccagtccgcactacacctatctgcactagatgcaaccttataaactcagaatactggtctatatcattatgagctgataagaaatgactagctcaagtccctatctgcactagatgcaaccttaataagtcctccgagtcattcttatcattcagacacattagtatgttaagtcactcacgatctatctgcactagatgcaaccttactactcaactatcttaacaactaatgtttagtagtcaatacagattatgacagcatagatctatctgcactagatgcaaccttatacaccacgacgcataatcgtattgattctttcactatatacttcaaggaccatcactacctatctgcactagatgcaaccttacactactaccagccttgaatatatagttcatgccctgtttattgacatgtcacatctcacctatctgcactagatgcaaccttacacatcacgcatcatgtcataaacagttatacaa

SEQ ID NO: 274 - RNA, 504 nt, synthetic construct
  source 1..504: mol_type = other RNA
tgatatacgaccagtccgcactacacctatctgcactagatgcagccttataaactcagaatactggtctatatcattatgagctgataagaaatgactagctcaagtccctatctgcactagatgcagccttaataagtcctccgagtcattcttatcattcagacacattagtatgttaagtcactcacgatctatctgcactagatgcagccttactactcaactatcttaacaactaatgtttagtagtcaatacagattatgacagcatagatctatctgcactagatgcagccttatacaccacgacgcataatcgtattgattctttcactatatacttcaaggaccatcactacctatctgcactagatgcagccttacactactaccagccttgaatatatagttcatgccctgtttattgacatgtcacatctcacctatctgcactagatgcagccttacacatcacgcatcatgtcataaacagttatacaa

SEQ ID NO: 275 - RNA, 504 nt, synthetic construct
  source 1..504: mol_type = other RNA
tgatatacgaccagtccgcactacacctatctgcactagatgcacccttataaactcagaatactggtctatatcattatgagctgataagaaatgactagctcaagtccctatctgcactagatgcacccttaataagtcctccgagtcattcttatcattcagacacattagtatgttaagtcactcacgatctatctgcactagatgcacccttactactcaactatcttaacaactaatgtttagtagtcaatacagattatgacagcatagatctatctgcactagatgcacccttatacaccacgacgcataatcgtattgattctttcactatatacttcaaggaccatcactacctatctgcactagatgcacccttacactactaccagccttgaatatatagttcatgccctgtttattgacatgtcacatctcacctatctgcactagatgcacccttacacatcacgcatcatgtcataaacagttatacaa

SEQ ID NO: 276 - RNA, 504 nt, synthetic construct
  source 1..504: mol_type = other RNA
tgatatacgaccagtccgcactacacctatctgcactagatgcatccttataaactcagaatactggtctatatcattatgagctgataagaaatgactagctcaagtccctatctgcactagatgcatccttaataagtcctccgagtcattcttatcattcagacacattagtatgttaagtcactcactatctatctgcactagatgcatccttactactcaactatcttaacaactaatgtttagtagtcaatacagattatgacagcatagacctatctgcactagatgcatccttatacaccacgacgcataatcgtattgattctttcactatatacttcaaggaccatcactacctatctgcactagatgcatccttacactactaccagccttgaatatatagttcatgccctgtttattgacatgtcacatctcacctatctgcactagatgcatccttacacatcacgcatcatgtcataaacagttatacaa

SEQ ID NO: 277 - RNA, 492 nt, synthetic construct
  source 1..492: mol_type = other RNA
tgatatacgaccagtccgcactacacctatctgcactactgcaccttataaactcagaatactggtctatatcattatgagctgataagaaatgacttcttcaagtccctatctgcactactgcaccttaataagtcctcctagtcattcttatcattcagacacattagtatgttaagtgactcatcatctatctgcactactgcaccttactactcaacgatcttaacaactaatgtttagtagtcaatacagattatgacagcatctatctatctgcactactgcaccttatacaccacgacgcataatcgtattgattctttcactatatacttcaaggaccatcactacctatctgcactactgcaccttacactactagcagccttgaatatatagttcatgccctgtttattgacatgtcacatctcacctatctgcactactgcaccttacacatcacgcatcatgtcataaacagttatacaa

SEQ ID NO: 278 - RNA, 498 nt, synthetic construct
  source 1..498: mol_type = other RNA
tgatatacgaccagtccgcactacacctatctgcactactgcaaccttataaactcagaatactggtctatatcattatgagctgataagaaatgacttcttcaagtccctatctgcactactgcaaccttaataagtcctcctagtcattcttatcattcagacacattagtatgttaagtcactcatcatctatctgcactactgcaaccttactactcaactgtcttaacaactaatgtttagtagtcaatacagattatgacagcatagatctatctgcactactgcaaccttatacaccacgacgcataatcgtattgattctttcactatatacttcaaggaccatcactacctatctgcactactgcaaccttacactactaccagccttgaatatatagttcatgccctgtttattgacatgtcacatctcacctatctgcactactgcaaccttacacatcacgcatcatgtcataaacagttatacaa

SEQ ID NO: 279 - RNA, 498 nt, synthetic construct
  source 1..498: mol_type = other RNA
tgatatacgaccagtccgcactacacctatctgcactactgcagccttataaactcagaatactggtctatatcattatgagctgataagaaatgacttcttcaagtccctatctgcactactgcagccttaataagtcctcctagtcattcttatcattcagacacattagtatgttaagtcactcatcatctatctgcactactgcagccttactactcaactgtcttaacaactaatgtttagtagtcaatacagattatgacagcatagatctatctgcactactgcagccttatacaccacgacgcataatcgtattgattctttcactatatacttcaaggaccatcactacctatctgcactactgcagccttacactactaccagccttgaatatatagttcatgccctgtttattgacatgtcacatctcacctatctgcactactgcagccttacacatcacgcatcatgtcataaacagttatacaa

SEQ ID NO: 280 - RNA, 498 nt, synthetic construct
  source 1..498: mol_type = other RNA
tgatatacgaccagtccgcactacacctatctgcactactgcacccttataaactcagaatactggtctatatcattatgagctgataagaaatgacttcttcaagtccctatctgcactactgcacccttaataagtcctcctagtcattcttatcattcagacacattagtatgttaagtcactcatcatctatctgcactactgcacccttactactcaactgtcttaacaactaatgtttagtagtcaatacagattatgacagcatagatctatctgcactactgcacccttatacaccacgacgcataatcgtattgattctttcactatatacttcaaggaccatcactacctatctgcactactgcacccttacactactaccagccttgaatatatagttcatgccctgtttattgacatgtcacatctcacctatctgcactactgcacccttacacatcacgcatcatgtcataaacagttatacaa

SEQ ID NO: 281 - RNA, 498 nt, synthetic construct
  source 1..498: mol_type = other RNA
tgatatacgaccagtccgcactacacctatctgcactactgcatccttataaactcagaatactggtctatatcattatgagctgataagaaatgacttcttcaagtccctatctgcactactgcatccttaataagtcctcctagtcattcttatcattcagacacattagtatgttaagtcactcatcatctatctgcactactgcatccttactactcaactgtcttaacaactaatgtttagtagtcaatacagattatgacagcatagatctatctgcactactgcatccttatacaccacgacgcataatcgtattgattctttcactatatacttcaaggaccatcactacctatctgcactactgcatccttacactactaccagccttgaatatatagttcatgccctgtttattgacatgtcacatctcacctatctgcactactgcatccttacacatcacgcatcatgtcataaacagttatacaa

SEQ ID NO: 282 - RNA, 498 nt, synthetic construct
  source 1..498: mol_type = other RNA
tgatatacgaccagtcctcactactactacctgcactgtaagcactttgacttcgccgattactggtctatatcattatgagctgataagaaatgactatcctgactacctacctgcactgtaagcactttgacatcctacctgagtcattcttatcattcagacacattagtatgttaagtcaagccacatctacctgcactgtaagcactttgctaagcgacactcttaacaactaatgtttagtagtcaatacagattatgacttccgatctctacctgcactgtaagcactttgtcctattacgtccataatcgtattgattctttcactatatacttcaaggaccatcacgaactacctgcactgtaagcactttgacctactaccatccttgaatatatagttcatgccctgtttattgacatgtcactccatacctacctgcactgtaagcactttgaccattcctcttcatgtcataaacagttatacaa

SEQ ID NO: 283 - RNA, 504 nt, synthetic construct
  source 1..504: mol_type = other RNA
tgatatacgaccagtcctcactactactacctgcactgtaagcaactttgacttcgccgattactggtctatatcattatgagctgataagaaatgactatcctgactacctacctgcactgtaagcaactttgacatcctacctgagtcattcttatcattcagacacattagtatgttaagtcaagccacatctacctgcactgtaagcaactttgctaagcgacactcttaacaactaatgtttagtagtcaatacagattatgacttccgatctctacctgcactgtaagcaactttgtcctattacgtccataatcgtattgattctttcactatatacttcaaggaccatcacgaactacctgcactgtaagcaactttgacctactaccatccttgaatatatagttcatgccctgtttattgacatgtcactccatacctacctgcactgtaagcaactttgaccattcctcttcatgtcataaacagttatacaa

SEQ ID NO: 284 - RNA, 504 nt, synthetic construct
  source 1..504: mol_type = other RNA
tgatatacgaccagtcctcactactactacctgcactgtaagcagctttgacttcgccgattactggtctatatcattatgagctgataagaaatgactatcctgactacctacctgcactgtaagcagctttgacatcctacctgagtcattcttatcattcagacacattagtatgttaagtcaagccacatctacctgcactgtaagcagctttgctaagcgacactcttaacaactaatgtttagtagtcaatacagattatgacttccgatctctacctgcactgtaagcagctttgtcctattacgtccataatcgtattgattctttcactatatacttcaaggaccatcacgaactacctgcactgtaagcagctttgacctactaccatccttgaatatatagttcatgccctgtttattgacatgtcactccatacctacctgcactgtaagcagctttgaccattcctcttcatgtcataaacagttatacaa

SEQ ID NO: 285 - RNA, 504 nt, synthetic construct
  source 1..504: mol_type = other RNA
tgatatacgaccagtcctcactactactacctgcactgtaagcacctttgacttcgccgattactggtctatatcattatgagctgataagaaatgactatcctgactacctacctgcactgtaagcacctttgacatcctacctgagtcattcttatcattcagacacattagtatgttaagtcaagccacatctacctgcactgtaagcacctttgctaagcgacactcttaacaactaatgtttagtagtcaatacagattatgacttccgatctctacctgcactgtaagcacctttgtcctattacgtccataatcgtattgattctttcactatatacttcaaggaccatcacgaactacctgcactgtaagcacctttgacctactaccatccttgaatatatagttcatgccctgtttattgacatgtcactccatacctacctgcactgtaagcacctttgaccattcctcttcatgtcataaacagttatacaa

SEQ ID NO: 286 - RNA, 504 nt, synthetic construct
  source 1..504: mol_type = other RNA
tgatatacgaccagtcctcactactactacctgcactgtaagcatctttgacttcgccgattactggtctatatcattatgagctgataagaaatgactatcctgactacctacctgcactgtaagcatctttgacatcctacctgagtcattcttatcattcagacacattagtatgttaagtcatgacacacctacctgcactgtaagcatctttgacatccgacactcttaacaactaatgtttagtagtcaatacagattatgacttccgatctctacctgcactgtaagcatctttgtcctattacgtccataatcgtattgattctttcactatatacttcaaggaccatcacgaactacctgcactgtaagcatctttgacctactaccatccttgaatatatagttcatgccctgtttattgacatgtcactccatacctacctgcactgtaagcatctttgaccattcctcttcatgtcataaacagttatacaa

SEQ ID NO: 287 - RNA, 492 nt, synthetic construct
  source 1..492: mol_type = other RNA
tgatatacgaccagtccgcactacacctacctgcactgatgcactttgtaaactcctgatactggtctatatcattatgagctgataagaaatgactatccgaactacctacctgcactgatgcactttgccatccaacctgagtcattcttatcattcagacacattagtatgttaagtgactcctcatctacctgcactgatgcactttgctactcaactatcttaacaactaatgtttagtagtcaatacagattatgacatcatatatctacctgcactgatgcactttgcccacctcgacgcataatcgtattgattctttcactatatacttcaaggaccatcactatctacctgcactgatgcactttgtcctactaccagccttgaatatatagttcatgccctgtttattgacatgtcacatctcacctacctgcactgatgcactttgcataacactcatcatgtcataaacagttatacaa

SEQ ID NO: 288 - RNA, 498 nt, synthetic construct
  source 1..498: mol_type = other RNA
tgatatacgaccagtccatacgacacctacctgcactgatgcaactttgctaactgatcatactggtctatatcattatgagctgataagaaatgactatccgaactacctacctgcactgatgcaactttgacatgctgactgagtcattcttatcattcagacacattagtatgttaagtgactccacatctacctgcactgatgcaactttgctacgcctctatcttaacaactaatgtttagtagtcaatacagattatgactttcacgccctacctgcactgatgcaactttgactactttaccgcataatcgtattgattctttcactatatacttcaaggagtctcacgctctacctgcactgatgcaactttgtactactaccagccttgaatatatagttcatgccctgtttattgacatgcgacaccactcctacctgcactgatgcaactttgctgaacactgatcatgtcataaacagttatacaa

SEQ ID NO: 289 - RNA, 498 nt, synthetic construct
  source 1..498: mol_type = other RNA
tgatatacgaccagtccatacgacacctacctgcactgatgcagctttgctaactgatcatactggtctatatcattatgagctgataagaaatgactatccgaactacctacctgcactgatgcagctttgacatgctgactgagtcattcttatcattcagacacattagtatgttaagtgactccacatctacctgcactgatgcagctttgctacgcctctatcttaacaactaatgtttagtagtcaatacagattatgactttcacgccctacctgcactgatgcagctttgactactttaccgcataatcgtattgattctttcactatatacttcaaggagtctcacgctctacctgcactgatgcagctttgtactactaccagccttgaatatatagttcatgccctgtttattgacatgcgacaccactcctacctgcactgatgcagctttgctgaacactgatcatgtcataaacagttatacaa

SEQ ID NO: 290 - RNA, 498 nt, synthetic construct
  source 1..498: mol_type = other RNA
tgatatacgaccagtccatacgacacctacctgcactgatgcacctttgctaactgatcatactggtctatatcattatgagctgataagaaatgactatccgaactacctacctgcactgatgcacctttgacatgctgactgagtcattcttatcattcagacacattagtatgttaagtgactccacatctacctgcactgatgcacctttgctacgcctctatcttaacaactaatgtttagtagtcaatacagattatgactttcacgccctacctgcactgatgcacctttgactactttaccgcataatcgtattgattctttcactatatacttcaaggagtctcacgctctacctgcactgatgcacctttgtactactaccagccttgaatatatagttcatgccctgtttattgacatgcgacaccactcctacctgcactgatgcacctttgctgaacactgatcatgtcataaacagttatacaa

SEQ ID NO: 291 - RNA, 498 nt, synthetic construct
  source 1..498: mol_type = other RNA
tgatatacgaccagtcctcactactactacctgcactgatgcatctttgacttcgccgattactggtctatatcattatgagctgataagaaatgactatcctgactacctacctgcactgatgcatctttgacctgctacctgagtcattcttatcattcagacacattagtatgttaagtcaagccacatctacctgcactgatgcatctttgctaagcgacactcttaacaactaatgtttagtagtcaatacagattatgacttccgatctctacctgcactgatgcatctttgtcctattacgtccataatcgtattgattctttcactatatacttcaaggaccttcacgaactacctgcactgatgcatctttgacctactaccatccttgaatatatagttcatgccctgtttattgacatgtcactccatacctacctgcactgatgcatctttgaccattcctcttcatgtcataaacagttatacaa

SEQ ID NO: 292 - RNA, 495 nt, synthetic construct
  source 1..495: mol_type = other RNA
gggtgatatacgaccagttcttattctcccgaccatggctgtagactgttaaatacttcaaatactggtctatatcattatgagctgataagaaatgacttcaaccaaatccgaccatggctgtagactgttaacaatatcccttagtcattcttatcattcagacacattagtatgttaagtatatcaaattcgaccatggctgtagactgttaatattctctcctcttaacaactaatgtttagtagtcaatacagattatgtacacataattcgaccatggctgtagactgttaataatcctatttcataatcgtattgattctttcactatatacttcaaggtattctcttctcgaccatggctgtagactgttaacacaacacactccttgaatatatagttcatgccctgtttattgacatgtactcatatttcgaccatggctgtagactgttaatttatcctattcatgtcataaacagttatacaa

SEQ ID NO: 293 - RNA, 501 nt, synthetic construct
  source 1..501: mol_type = other RNA
gggtgatatacgaccagtatagactacaacgaccatggctgtagacatgttagaaactcaagatactggtctatatcattatgagctgataagaaatgactcaacttacgtacgaccatggctgtagacatgttacaatatcacctcagtcattcttatcacttagacacattagtatgttaagctcatccttatcgaccatggctgtagacatgttacactcaaatcatcttaacaactaatgtttagtagtcaatacagattatgactctatcaaacgaccatggctgtagacatgttagcaacatcatcacataatcgtattgacatgcaatctatatacttcaaggaagatgactaacgaccatggctgtagacatgttacactgctagtaaccttgaatatatagctgtagttctgtttattgacatgtcaacatcaatcgaccatggctgtagacatgttacactttacctaacatgtcataaacagtaactgaa

SEQ ID NO: 294 - RNA, 501 nt, synthetic construct
  source 1..501: mol_type = other RNA
gggtgatatacgaccagtatagactacaacgaccatggctgtagacgtgttagaaactcaagatactggtctatatcattatgagctgataagaaatgactcaacttacgtacgaccatggctgtagacgtgttacaatatcacctcagtcattcttatcacttagacacattagtatgttaagctcatccttatcgaccatggctgtagacgtgttacactcaaatcatcttaacaactaatgtttagtagtcaatacagattatgactctatcaaacgaccatggctgtagacgtgttagcatcatcatcacataatcgtattgacatgcaatctatatacttcaaggaagatgactaacgaccatggctgtagacgtgttacactgctagtaaccttgaatatatagctgaagttctgtttattgacatgtcaacatcaatcgaccatggctgtagacgtgttacactttacctaacatgtcataaacagtaaccgaa

SEQ ID NO: 295 - RNA, 501 nt, synthetic construct
  source 1..501: mol_type = other RNA
gggtgatatacgaccagtcacatacctcccgaccatggctgtagacctgttaatactctcacatactggtctatatcattatgagctgataagaaatgactactattcacaacgaccatggctgtagacctgttaaatccaaattcaagtcattcttatcattcagacacattagtatgttaagcctcacttccccgaccatggctgtagacctgttaaacattccacctcttaacaactaatgtttagtagtcaatacagattatgcaccctatcaacgaccatggctgtagacctgttaaacatacacactcataatcgtattgattctttcactatatacttcaaggccttcttctatcgaccatggctgtagacctgttaacctcactaataccttgaatatatagttcatgccctgtttattgacatgacactccctatcgaccatggctgtagacctgttacttcttctatcacatgtcataaacagttatacaa

SEQ ID NO: 296 - RNA, 501 nt, synthetic construct
  source 1..501: mol_type = other RNA
gggtgatatacgaccagttacttcactctcgaccatggctgtagacttgttacttacttcaactactggtctatatcatcttgagctgataagaaatgactcatactaacttcgaccatggctgtagacttgttaaccttatcattcagtcattcttatcacttagacacattagtatgttaagctcttactaatcgaccatggctgtagacttgttactaacttaccctcttaacaactaatgtttagtagtcaatacagattatgtcattccctatcgaccatggctgtagacttgttaactcttacctcacataatcgtattgacatgcaatctatatacttcaaggtttcatcccttcgaccatggctgtagacttgttaaatcccttcattccttgaatatatagctttagttctgtttattgacatgtccactttaatcgaccatggctgtagacttgttaccaactttcaatcatgtcataaacagtaaccgaa

SEQ ID NO: 297 - RNA, 488 nt, synthetic construct
  source 1..488: mol_type = other RNA
gggtgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagacacattagtatgttaagctcatccttatcgaccatggctcagactgttacactcaaatcatcttaacaactaatgtttagtagtcaatacagattatgactctatcaaacgaccatggctcagactgttagcaacatcatccataatcgtattgacttgcaatctatatacttcaaggaagatcactaacgaccatggctcagactgttacactactaccaaccttgaatatatagccgtagttctgtttattgacatgtcaacatcaatcgaccatggctcagactgttacactttacctaacatgtcataaacagtaacagaa

SEQ ID NO: 298 - RNA, 495 nt, synthetic construct
  source 1..495: mol_type = other RNA
gggtgatatacgaccagtatagactacaacgaccatggctcagacatgttagaaactcaagatactggtctatatcattatgagctgataagaaatgactcaacttacgtacgaccatggctcagacatgttacaatatcacctcagtcattcttatcacttagacacattagtatgttaagctcatccttatcgaccatggctcagacatgttacactcaaatcatcttaacaactaatgtttagtagtcaatacagattatgactctatcaaacgaccatggctcagacatgttagcaacatcatcacataatcgtattgacttgcaatctatatacttcaaggaagatcactaacgaccatggctcagacatgttacactactaccaaccttgaatatatagccgtagttctgtttattgacatgtcaacatcaatcgaccatggctcagacatgttacactttacctaacatgtcataaacagtaacagaa

SEQ ID NO: 299 - RNA, 495 nt, synthetic construct
  source 1..495: mol_type = other RNA
gggtgatatacgaccagtatagactacaacgaccatggctcagacgtgttagaaactcaagatactggtctatatcattatgagctgataagaaatgactcaacttacgtacgaccatggctcagacgtgttacaatatcacctcagtcattcttatcacttagacacattagtatgttaagctcatccttatcgaccatggctcagacgtgttacactcaaatcatcttaacaactaatgtttagtagtcaatacagattatgactctatcaaacgaccatggctcagacgtgttagcaacatcatcacataatcgtattgacttgcaatctatatacttcaaggaagatcactaacgaccatggctcagacgtgttacactactaccaaccttgaatatatagccgtagttctgtttattgacatgtcaacatcaatcgaccatggctcagacgtgttacactttacctaacatgtcataaacagtaacagaa

SEQ ID NO: 300 - RNA, 495 nt, synthetic construct
  source 1..495: mol_type = other RNA
gggtgatatacgaccagtatagactacaacgaccatggctcagacctgttagaaactcaagatactggtctatatcattatgagctgataagaaatgactcaacttacgtacgaccatggctcagacctgttacaatatcacctcagtcattcttatcacttagacacattagtatgttaagctcatccttatcgaccatggctcagacctgttacactcaaatcatcttaacaactaatgtttagtagtcaatacagattatgactctatcaaacgaccatggctcagacctgttagcaacatcatcacataatcgtattgacttgcaatctatatacttcaaggaagatcactaacgaccatggctcagacctgttacactactaccaaccttgaatatatagccgtagttctgtttattgacatgtcaacatcaatcgaccatggctcagacctgttacactttacctaacatgtcataaacagtaacagaa

SEQ ID NO: 301 - RNA, 495 nt, synthetic construct
  source 1..495: mol_type = other RNA
gggtgatatacgaccagtatagactacaacgaccatggctcagacttgttagaaactcaagatactggtctatatcattatgagctgataagaaatgactcaacttacctacgaccatggctcagacttgttacaatatcacctcagtcattcttatcacttagacacattagtatgttaagcttacgcataccgaccatggctcagacttgttacactctaatcctcttaacaactaatgtttagtagtcaatacagattatgactctatcaaacgaccatggctcagacttgttagcaacatcatcacataatcgtattgacttgcaatctatatacttcaaggatcctcacactcgaccatggctcagacttgttacactacatccatccttgaatatatagccgtagttctgtttattgacatgtcaacatcaatcgaccatggctcagacttgttacactttacctaacatgtcataaacagtaacagaa

SEQ ID NO: 302 - RNA, 513 nt, synthetic construct
  source 1..513: mol_type = other RNA
gggtgatatacgaccagtgcgcactacacaacccctatcacgattagcaattaagaaactcaagatactggtctatatcattatgagctgataagaaatgactactttaactacaacccctatcacgattagcaattaagaacacgacatcagtcattcttatcattcagacacattagtatgttaagttaagcatcataacccctatcacgattagcaattaacaacgaaacgatcttaacaactaatgtttagtagtcaatacagattatgacagcatctataacccctatcacgattagcaattaatacaccacgacgcataatcgtattgattctttcactatatacttcaaggaccatcactacaacccctatcacgattagcaattaacactactagcagccttgaatatatagttcatgccctgtttattgacatgtcacatctcacaacccctatcacgattagcaattaacacatcacgcatcatgtcataaacagttatacaa

SEQ ID NO: 303 - RNA, 513 nt, synthetic construct
  source 1..513: mol_type = other RNA
gggtgatatacgaccagttcatactacacaacccctatcacgattagcgattaagaaactcaatatactggtctatatcattatgagctgataagaaatgactacaccgacataaacccctatcacgattagcgattaacctacagagattagtcattcttatcattcagacacattagtatgttaagtgactcatcataacccctatcacgattagcgattaaattcagaacgatcttaacaactaatgtttagtagtcaatacagattatgacagcaactataacccctatcacgattagcgattaatacaccacgaatcataatcgtattgattctttcactatatacttcaaggacatacctatcaacccctatcacgattagcgattaacaccatgaccagccttgaatatatagttcatgccctgtttattgacatgtcacatctcacaacccctatcacgattagcgattaacactccactcatcatgtcataaacagttatacaa

SEQ ID NO: 304 - RNA, 513 nt, synthetic construct
  source 1..513: mol_type = other RNA
gggtgatatacgaccagtgcgcactacacaacccctatcacgattagccattaagaaactcaagatactggtctatatcattatgagctgataagaaatgactactttaactacaacccctatcacgattagccattaagaacacgacatcagtcattcttatcattcagacacattagtatgttaagttaagcatcataacccctatcacgattagccattaacaacgaaacgatcttaacaactaatgtttagtagtcaatacagattatgacagcatctataacccctatcacgattagccattaatacaccacgacgcataatcgtattgattctttcactatatacttcaaggaccatcactacaacccctatcacgattagccattaacactactagcagccttgaatatatagttcatgccctgtttattgacatgtcacatctcacaacccctatcacgattagccattaacacatcacgcatcatgtcataaacagttatacaa

SEQ ID NO: 305 - RNA, 513 nt, synthetic construct
  source 1..513: mol_type = other RNA
gggtgatatacgaccagtgcgcactacacaacccctatcacgattagctattaagaaactcaagatactggtctatatcattatgagctgataagaaatgactactttaactacaacccctatcacgattagctattaagaacacgacatcagtcattcttatcattcagacacattagtatgttaagttaccgatcataacccctatcacgattagctattaacttacaaacgatcttaacaactaatgtttagtagtcaatacagattatgacatcacgcataacccctatcacgattagctattaatacaccacgacgcataatcgtattgattctttcactatatacttcaaggactctatctacaacccctatcacgattagctattaacacttcacgcctccttgaatatatagttcatgccctgtttattgacatgtcacatctcacaacccctatcacgattagctattaacacatcacgcatcatgtcataaacagttatacaa

SEQ ID NO: 306 - RNA, 501 nt, synthetic construct
  source 1..501: mol_type = other RNA
gggtgatatacgaccagtgcgcactacacaacccctatcacgtaagcattaagaaactcaagatactggtctatatcattatgagctgataagaaatgactatccgaactacaacccctatcacgtaagcattaatcatccaacatgagtcattcttatcattcagacacattagtatgttaagtgactcatcataacccctatcacgtaagcattaactactcaacgatcttaacaactaatgtttagtagtcaatacagattatgacagcatctataacccctatcacgtaagcattaatacaccacgacgcataatcgtattgattctttcactatatacttcaaggaccatcactacaacccctatcacgtaagcattaacactactagcagccttgaatatatagttcatgccctgtttattgacatgtcacatctcacaacccctatcacgtaagcattaacacatcacgcatcatgtcataaacagttatacaa

SEQ ID NO: 307 - RNA, 507 nt, synthetic construct
  source 1..507: mol_type = other RNA
gggtgatatacgaccagtgcgcactacacaacccctatcacgtaagcaattaagaaactcaagatactggtctatatcattatgagctgataagaaatgactatccgaactacaacccctatcacgtaagcaattaatcatccaacatgagtcattcttatcattcagacacattagtatgttaagtgactcatcataacccctatcacgtaagcaattaactactcaacgatcttaacaactaatgtttagtagtcaatacagattatgacagcatctataacccctatcacgtaagcaattaatacaccacgacgcataatcgtattgattctttcactatatacttcaaggaccatcactacaacccctatcacgtaagcaattaacactactagcagccttgaatatatagttcatgccctgtttattgacatgtcacatctcacaacccctatcacgtaagcaattaacacatcacgcatcatgtcataaacagttatacaa

SEQ ID NO: 308 - RNA, 507 nt, synthetic construct
  source 1..507: mol_type = other RNA
gggtgatatacgaccagttcagactacacaacccctatcacgtaagcgattaagaaactcaatatactggtctatatcattatgagctgataagaaatgactacaccgacataaacccctatcacgtaagcgattaacctacagagattagtcattcttatcattcagacacattagtatgttaagtgactcagcataacccctatcacgtaagcgattaaattcagaacaatcttaacaactaatgtttagtagtcaatacagattatgacagcaactataacccctatcacgtaagcgattaatacaccacgaatcataatcgtattgattctttcactatatacttcaaggacatacctatcaacccctatcacgtaagcgattaacaccatcaccagccttgaatatatagttcatgccctgtttattgacatgtcacatctcacaacccctatcacgtaagcgattaacactccactcatcatgtcataaacagttatacaa

SEQ ID NO: 309 - RNA, 507 nt, synthetic construct
  source 1..507: mol_type = other RNA
gggtgatatacgaccagtgcgcactacacaacccctatcacgtaagccattaagaaactcaagatactggtctatatcattatgagctgataagaaatgactatccgaactacaacccctatcacgtaagccattaatcatccaacatgagtcattcttatcattcagacacattagtatgttaagtgactcatcataacccctatcacgtaagccattaactactcaacgatcttaacaactaatgtttagtagtcaatacagattatgacagcatctataacccctatcacgtaagccattaatacaccacgacgcataatcgtattgattctttcactatatacttcaaggaccatcactacaacccctatcacgtaagccattaacactactagcagccttgaatatatagttcatgccctgtttattgacatgtcacatctcacaacccctatcacgtaagccattaacacatcacgcatcatgtcataaacagttatacaa

SEQ ID NO: 310 - RNA, 507 nt, synthetic construct
  source 1..507: mol_type = other RNA
gggtgatatacgaccagtgcgcactacacaacccctatcacgtaagctattaagaaactcaagatactggtctatatcattatgagctgataagaaatgactatccgaactacaacccctatcacgtaagctattaatcatccaacatgagtcattcttatcattcagacacattagtatgttaagtgactcatcataacccctatcacgtaagctattaactactcaacgatcttaacaactaatgtttagtagtcaatacagattatgacaccatctataacccctatcacgtaagctattaatacaccacgacgcataatcgtattgattctttcactatatacttcaaggaccatcacaacaacccctatcacgtaagctattaacacgaccacgagccttgaatatatagttcatgccctgtttattgacatgtcacatctcacaacccctatcacgtaagctattaacacatcacgcatcatgtcataaacagttatacaa

SEQ ID NO: 311 - RNA, 501 nt, synthetic construct
  source 1..501: mol_type = other RNA
gggtgatatacgaccagtccgcactacacctatctgcactagatgcaccttataaactcagaatactggtctatatcattatgagctgataagaaatgactagctcaagtccctatctgcactagatgcaccttaataagtcctccgagtcattcttatcattcagacacattagtatgttaagtcactcacgatctatctgcactagatgcaccttactactcaactatcttaacaactaatgtttagtagtcaatacagattatgacagcatagatctatctgcactagatgcaccttatacaccacgacgcataatcgtattgattctttcactatatacttcaaggaccatcactacctatctgcactagatgcaccttacactactaccagccttgaatatatagttcatgccctgtttattgacatgtcacatctcacctatctgcactagatgcaccttacacatcacgcatcatgtcataaacagttatacaa

SEQ ID NO: 312 - RNA, 507 nt, synthetic construct
  source 1..507: mol_type = other RNA
gggtgatatacgaccagtccgcactacacctatctgcactagatgcaaccttataaactcagaatactggtctatatcattatgagctgataagaaatgactagctcaagtccctatctgcactagatgcaaccttaataagtcctccgagtcattcttatcattcagacacattagtatgttaagtcactcacgatctatctgcactagatgcaaccttactactcaactatcttaacaactaatgtttagtagtcaatacagattatgacagcatagatctatctgcactagatgcaaccttatacaccacgacgcataatcgtattgattctttcactatatacttcaaggaccatcactacctatctgcactagatgcaaccttacactactaccagccttgaatatatagttcatgccctgtttattgacatgtcacatctcacctatctgcactagatgcaaccttacacatcacgcatcatgtcataaacagttatacaa

SEQ ID NO: 313 - RNA, 507 nt, synthetic construct
  source 1..507: mol_type = other RNA
gggtgatatacgaccagtccgcactacacctatctgcactagatgcagccttataaactcagaatactggtctatatcattatgagctgataagaaatgactagctcaagtccctatctgcactagatgcagccttaataagtcctccgagtcattcttatcattcagacacattagtatgttaagtcactcacgatctatctgcactagatgcagccttactactcaactatcttaacaactaatgtttagtagtcaatacagattatgacagcatagatctatctgcactagatgcagccttatacaccacgacgcataatcgtattgattctttcactatatacttcaaggaccatcactacctatctgcactagatgcagccttacactactaccagccttgaatatatagttcatgccctgtttattgacatgtcacatctcacctatctgcactagatgcagccttacacatcacgcatcatgtcataaacagttatacaa

SEQ ID NO: 314 - RNA, 507 nt, synthetic construct
  source 1..507: mol_type = other RNA
gggtgatatacgaccagtccgcactacacctatctgcactagatgcacccttataaactcagaatactggtctatatcattatgagctgataagaaatgactagctcaagtccctatctgcactagatgcacccttaataagtcctccgagtcattcttatcattcagacacattagtatgttaagtcactcacgatctatctgcactagatgcacccttactactcaactatcttaacaactaatgtttagtagtcaatacagattatgacagcatagatctatctgcactagatgcacccttatacaccacgacgcataatcgtattgattctttcactatatacttcaaggaccatcactacctatctgcactagatgcacccttacactactaccagccttgaatatatagttcatgccctgtttattgacatgtcacatctcacctatctgcactagatgcacccttacacatcacgcatcatgtcataaacagttatacaa

SEQ ID NO: 315 - RNA, 507 nt, synthetic construct
  source 1..507: mol_type = other RNA
gggtgatatacgaccagtccgcactacacctatctgcactagatgcatccttataaactcagaatactggtctatatcattatgagctgataagaaatgactagctcaagtccctatctgcactagatgcatccttaataagtcctccgagtcattcttatcattcagacacattagtatgttaagtcactcactatctatctgcactagatgcatccttactactcaactatcttaacaactaatgtttagtagtcaatacagattatgacagcatagacctatctgcactagatgcatccttatacaccacgacgcataatcgtattgattctttcactatatacttcaaggaccatcactacctatctgcactagatgcatccttacactactaccagccttgaatatatagttcatgccctgtttattgacatgtcacatctcacctatctgcactagatgcatccttacacatcacgcatcatgtcataaacagttatacaa

SEQ ID NO: 316 - RNA, 495 nt, synthetic construct
  source 1..495: mol_type = other RNA
gggtgatatacgaccagtccgcactacacctatctgcactactgcaccttataaactcagaatactggtctatatcattatgagctgataagaaatgacttcttcaagtccctatctgcactactgcaccttaataagtcctcctagtcattcttatcattcagacacattagtatgttaagtgactcatcatctatctgcactactgcaccttactactcaacgatcttaacaactaatgtttagtagtcaatacagattatgacagcatctatctatctgcactactgcaccttatacaccacgacgcataatcgtattgattctttcactatatacttcaaggaccatcactacctatctgcactactgcaccttacactactagcagccttgaatatatagttcatgccctgtttattgacatgtcacatctcacctatctgcactactgcaccttacacatcacgcatcatgtcataaacagttatacaa

SEQ ID NO: 317 - RNA, 501 nt, synthetic construct
  source 1..501: mol_type = other RNA
gggtgatatacgaccagtccgcactacacctatctgcactactgcaaccttataaactcagaatactggtctatatcattatgagctgataagaaatgacttcttcaagtccctatctgcactactgcaaccttaataagtcctcctagtcattcttatcattcagacacattagtatgttaagtcactcatcatctatctgcactactgcaaccttactactcaactgtcttaacaactaatgtttagtagtcaatacagattatgacagcatagatctatctgcactactgcaaccttatacaccacgacgcataatcgtattgattctttcactatatacttcaaggaccatcactacctatctgcactactgcaaccttacactactaccagccttgaatatatagttcatgccctgtttattgacatgtcacatctcacctatctgcactactgcaaccttacacatcacgcatcatgtcataaacagttatacaa

SEQ ID NO: 318 - RNA, 501 nt, synthetic construct
  source 1..501: mol_type = other RNA
gggtgatatacgaccagtccgcactacacctatctgcactactgcagccttataaactcagaatactggtctatatcattatgagctgataagaaatgacttcttcaagtccctatctgcactactgcagccttaataagtcctcctagtcattcttatcattcagacacattagtatgttaagtcactcatcatctatctgcactactgcagccttactactcaactgtcttaacaactaatgtttagtagtcaatacagattatgacagcatagatctatctgcactactgcagccttatacaccacgacgcataatcgtattgattctttcactatatacttcaaggaccatcactacctatctgcactactgcagccttacactactaccagccttgaatatatagttcatgccctgtttattgacatgtcacatctcacctatctgcactactgcagccttacacatcacgcatcatgtcataaacagttatacaa

SEQ ID NO: 319 - RNA, 501 nt, synthetic construct
  source 1..501: mol_type = other RNA
gggtgatatacgaccagtccgcactacacctatctgcactactgcacccttataaactcagaatactggtctatatcattatgagctgataagaaatgacttcttcaagtccctatctgcactactgcacccttaataagtcctcctagtcattcttatcattcagacacattagtatgttaagtcactcatcatctatctgcactactgcacccttactactcaactgtcttaacaactaatgtttagtagtcaatacagattatgacagcatagatctatctgcactactgcacccttatacaccacgacgcataatcgtattgattctttcactatatacttcaaggaccatcactacctatctgcactactgcacccttacactactaccagccttgaatatatagttcatgccctgtttattgacatgtcacatctcacctatctgcactactgcacccttacacatcacgcatcatgtcataaacagttatacaa

SEQ ID NO: 320 - RNA, 501 nt, synthetic construct
  source 1..501: mol_type = other RNA
gggtgatatacgaccagtccgcactacacctatctgcactactgcatccttataaactcagaatactggtctatatcattatgagctgataagaaatgacttcttcaagtccctatctgcactactgcatccttaataagtcctcctagtcattcttatcattcagacacattagtatgttaagtcactcatcatctatctgcactactgcatccttactactcaactgtcttaacaactaatgtttagtagtcaatacagattatgacagcatagatctatctgcactactgcatccttatacaccacgacgcataatcgtattgattctttcactatatacttcaaggaccatcactacctatctgcactactgcatccttacactactaccagccttgaatatatagttcatgccctgtttattgacatgtcacatctcacctatctgcactactgcatccttacacatcacgcatcatgtcataaacagttatacaa

SEQ ID NO: 321 - RNA, 501 nt, synthetic construct
  source 1..501: mol_type = other RNA
gggtgatatacgaccagtcctcactactactacctgcactgtaagcactttgacttcgccgattactggtctatatcattatgagctgataagaaatgactatcctgactacctacctgcactgtaagcactttgacatcctacctgagtcattcttatcattcagacacattagtatgttaagtcaagccacatctacctgcactgtaagcactttgctaagcgacactcttaacaactaatgtttagtagtcaatacagattatgacttccgatctctacctgcactgtaagcactttgtcctattacgtccataatcgtattgattctttcactatatacttcaaggaccatcacgaactacctgcactgtaagcactttgacctactaccatccttgaatatatagttcatgccctgtttattgacatgtcactccatacctacctgcactgtaagcactttgaccattcctcttcatgtcataaacagttatacaa

SEQ ID NO: 322 - RNA, 507 nt, synthetic construct
  source 1..507: mol_type = other RNA
gggtgatatacgaccagtcctcactactactacctgcactgtaagcaactttgacttcgccgattactggtctatatcattatgagctgataagaaatgactatcctgactacctacctgcactgtaagcaactttgacatcctacctgagtcattcttatcattcagacacattagtatgttaagtcaagccacatctacctgcactgtaagcaactttgctaagcgacactcttaacaactaatgtttagtagtcaatacagattatgacttccgatctctacctgcactgtaagcaactttgtcctattacgtccataatcgtattgattctttcactatatacttcaaggaccatcacgaactacctgcactgtaagcaactttgacctactaccatccttgaatatatagttcatgccctgtttattgacatgtcactccatacctacctgcactgtaagcaactttgaccattcctcttcatgtcataaacagttatacaa

SEQ ID NO: 323 - RNA, 507 nt, synthetic construct
  source 1..507: mol_type = other RNA
gggtgatatacgaccagtcctcactactactacctgcactgtaagcagctttgacttcgccgattactggtctatatcattatgagctgataagaaatgactatcctgactacctacctgcactgtaagcagctttgacatcctacctgagtcattcttatcattcagacacattagtatgttaagtcaagccacatctacctgcactgtaagcagctttgctaagcgacactcttaacaactaatgtttagtagtcaatacagattatgacttccgatctctacctgcactgtaagcagctttgtcctattacgtccataatcgtattgattctttcactatatacttcaaggaccatcacgaactacctgcactgtaagcagctttgacctactaccatccttgaatatatagttcatgccctgtttattgacatgtcactccatacctacctgcactgtaagcagctttgaccattcctcttcatgtcataaacagttatacaa

SEQ ID NO: 324 - RNA, 507 nt, synthetic construct
  source 1..507: mol_type = other RNA
gggtgatatacgaccagtcctcactactactacctgcactgtaagcacctttgacttcgccgattactggtctatatcattatgagctgataagaaatgactatcctgactacctacctgcactgtaagcacctttgacatcctacctgagtcattcttatcattcagacacattagtatgttaagtcaagccacatctacctgcactgtaagcacctttgctaagcgacactcttaacaactaatgtttagtagtcaatacagattatgacttccgatctctacctgcactgtaagcacctttgtcctattacgtccataatcgtattgattctttcactatatacttcaaggaccatcacgaactacctgcactgtaagcacctttgacctactaccatccttgaatatatagttcatgccctgtttattgacatgtcactccatacctacctgcactgtaagcacctttgaccattcctcttcatgtcataaacagttatacaa

SEQ ID NO: 325 - RNA, 507 nt, synthetic construct
  source 1..507: mol_type = other RNA
gggtgatatacgaccagtcctcactactactacctgcactgtaagcatctttgacttcgccgattactggtctatatcattatgagctgataagaaatgactatcctgactacctacctgcactgtaagcatctttgacatcctacctgagtcattcttatcattcagacacattagtatgttaagtcatgacacacctacctgcactgtaagcatctttgacatccgacactcttaacaactaatgtttagtagtcaatacagattatgacttccgatctctacctgcactgtaagcatctttgtcctattacgtccataatcgtattgattctttcactatatacttcaaggaccatcacgaactacctgcactgtaagcatctttgacctactaccatccttgaatatatagttcatgccctgtttattgacatgtcactccatacctacctgcactgtaagcatctttgaccattcctcttcatgtcataaacagttatacaa

SEQ ID NO: 326 - RNA, 495 nt, synthetic construct
  source 1..495: mol_type = other RNA
gggtgatatacgaccagtccgcactacacctacctgcactgatgcactttgtaaactcctgatactggtctatatcattatgagctgataagaaatgactatccgaactacctacctgcactgatgcactttgccatccaacctgagtcattcttatcattcagacacattagtatgttaagtgactcctcatctacctgcactgatgcactttgctactcaactatcttaacaactaatgtttagtagtcaatacagattatgacatcatatatctacctgcactgatgcactttgcccacctcgacgcataatcgtattgattctttcactatatacttcaaggaccatcactatctacctgcactgatgcactttgtcctactaccagccttgaatatatagttcatgccctgtttattgacatgtcacatctcacctacctgcactgatgcactttgcataacactcatcatgtcataaacagttatacaa

SEQ ID NO: 327 - RNA, 501 nt, synthetic construct
  source 1..501: mol_type = other RNA
gggtgatatacgaccagtccatacgacacctacctgcactgatgcaactttgctaactgatcatactggtctatatcattatgagctgataagaaatgactatccgaactacctacctgcactgatgcaactttgacatgctgactgagtcattcttatcattcagacacattagtatgttaagtgactccacatctacctgcactgatgcaactttgctacgcctctatcttaacaactaatgtttagtagtcaatacagattatgactttcacgccctacctgcactgatgcaactttgactactttaccgcataatcgtattgattctttcactatatacttcaaggagtctcacgctctacctgcactgatgcaactttgtactactaccagccttgaatatatagttcatgccctgtttattgacatgcgacaccactcctacctgcactgatgcaactttgctgaacactgatcatgtcataaacagttatacaa

SEQ ID NO: 328 - RNA, 501 nt, synthetic construct
  source 1..501: mol_type = other RNA
gggtgatatacgaccagtccatacgacacctacctgcactgatgcagctttgctaactgatcatactggtctatatcattatgagctgataagaaatgactatccgaactacctacctgcactgatgcagctttgacatgctgactgagtcattcttatcattcagacacattagtatgttaagtgactccacatctacctgcactgatgcagctttgctacgcctctatcttaacaactaatgtttagtagtcaatacagattatgactttcacgccctacctgcactgatgcagctttgactactttaccgcataatcgtattgattctttcactatatacttcaaggagtctcacgctctacctgcactgatgcagctttgtactactaccagccttgaatatatagttcatgccctgtttattgacatgcgacaccactcctacctgcactgatgcagctttgctgaacactgatcatgtcataaacagttatacaa

SEQ ID NO: 329 - RNA, 501 nt, synthetic construct
  source 1..501: mol_type = other RNA
gggtgatatacgaccagtccatacgacacctacctgcactgatgcacctttgctaactgatcatactggtctatatcattatgagctgataagaaatgactatccgaactacctacctgcactgatgcacctttgacatgctgactgagtcattcttatcattcagacacattagtatgttaagtgactccacatctacctgcactgatgcacctttgctacgcctctatcttaacaactaatgtttagtagtcaatacagattatgactttcacgccctacctgcactgatgcacctttgactactttaccgcataatcgtattgattctttcactatatacttcaaggagtctcacgctctacctgcactgatgcacctttgtactactaccagccttgaatatatagttcatgccctgtttattgacatgcgacaccactcctacctgcactgatgcacctttgctgaacactgatcatgtcataaacagttatacaa

SEQ ID NO: 330 - RNA, 501 nt, synthetic construct
  source 1..501: mol_type = other RNA
gggtgatatacgaccagtcctcactactactacctgcactgatgcatctttgacttcgccgattactggtctatatcattatgagctgataagaaatgactatcctgactacctacctgcactgatgcatctttgacctgctacctgagtcattcttatcattcagacacattagtatgttaagtcaagccacatctacctgcactgatgcatctttgctaagcgacactcttaacaactaatgtttagtagtcaatacagattatgacttccgatctctacctgcactgatgcatctttgtcctattacgtccataatcgtattgattctttcactatatacttcaaggaccttcacgaactacctgcactgatgcatctttgacctactaccatccttgaatatatagttcatgccctgtttattgacatgtcactccatacctacctgcactgatgcatctttgaccattcctcttcatgtcataaacagttatacaa

SEQ ID NO: 331 - RNA, 23 nt, synthetic construct
  source 1..23: mol_type = other RNA
ctacctgcactgtaagcactttg

SEQ ID NO: 332 - RNA, 24 nt, synthetic construct
  source 1..24: mol_type = other RNA
ctacctgcactgtaagcaactttg

SEQ ID NO: 333 - RNA, 24 nt, synthetic construct
  source 1..24: mol_type = other RNA
ctacctgcactgtaagcagctttg

SEQ ID NO: 334 - RNA, 24 nt, synthetic construct
  source 1..24: mol_type = other RNA
ctacctgcactgtaagcacctttg

SEQ ID NO: 335 - RNA, 24 nt, synthetic construct
  source 1..24: mol_type = other RNA
ctacctgcactgtaagcatctttg

SEQ ID NO: 336 - RNA, 22 nt, synthetic construct
  source 1..22: mol_type = other RNA
ctacctgcactgatgcactttg

SEQ ID NO: 337 - RNA, 23 nt, synthetic construct
  source 1..23: mol_type = other RNA
ctacctgcactgatgcaactttg

SEQ ID NO: 338 - RNA, 23 nt, synthetic construct
  source 1..23: mol_type = other RNA
ctacctgcactgatgcagctttg

SEQ ID NO: 339 - RNA, 23 nt, synthetic construct
  source 1..23: mol_type = other RNA
ctacctgcactgatgcacctttg

SEQ ID NO: 340 - RNA, 23 nt, synthetic construct
  source 1..23: mol_type = other RNA
ctacctgcactgatgcatctttg

SEQ ID NO: 341 - RNA, 23 nt, synthetic construct
  source 1..23: mol_type = other RNA
ctacctgcactgtaagcacttag

SEQ ID NO: 342 - RNA, 23 nt, synthetic construct
  source 1..23: mol_type = other RNA
ctacctgcactgtaagcactatg

SEQ ID NO: 343 - RNA, 23 nt, synthetic construct
  source 1..23: mol_type = other RNA
ctacctgcactgtaagcacattg

SEQ ID NO: 344 - RNA, 23 nt, synthetic construct
  source 1..23: mol_type = other RNA
ctacctgcactgtaagcagtttg

SEQ ID NO: 345 - RNA, 23 nt, synthetic construct
  source 1..23: mol_type = other RNA
ctacctgcactgtaagctctttg

SEQ ID NO: 346 - RNA, 23 nt, synthetic construct
  source 1..23: mol_type = other RNA
ctacctgcactgtaaggactttg

SEQ ID NO: 347 - RNA, 23 nt, synthetic construct
  source 1..23: mol_type = other RNA
ctacctgcactgtaaccactttg

SEQ ID NO: 348 - RNA, 22 nt, synthetic construct
  source 1..22: mol_type = other RNA
ctacctgcactgatgcacttag

SEQ ID NO: 349 - RNA, 22 nt, synthetic construct
  source 1..22: mol_type = other RNA
ctacctgcactgatgcactatg

SEQ ID NO: 350 - RNA, 22 nt, synthetic construct
  source 1..22: mol_type = other RNA
ctacctgcactgatgcacattg

SEQ ID NO: 351 - RNA, 22 nt, synthetic construct
  source 1..22: mol_type = other RNA
ctacctgcactgatgcagtttg

SEQ ID NO: 352 - RNA, 22 nt, synthetic construct
  source 1..22: mol_type = other RNA
ctacctgcactgatgctctttg

SEQ ID NO: 353 - RNA, 22 nt, synthetic construct
  source 1..22: mol_type = other RNA
ctacctgcactgatggactttg

SEQ ID NO: 354 - RNA, 22 nt, synthetic construct
  source 1..22: mol_type = other RNA
ctacctgcactgatccactttg

SEQ ID NO: 355 - RNA, 23 nt, synthetic construct
  source 1..23: mol_type = other RNA
ctatctgcactagatgcacctta

SEQ ID NO: 356 - RNA, 24 nt, synthetic construct
  source 1..24: mol_type = other RNA
ctatctgcactagatgcaacctta

SEQ ID NO: 357 - RNA, 24 nt, synthetic construct
  source 1..24: mol_type = other RNA
ctatctgcactagatgcagcctta

SEQ ID NO: 358 - RNA, 24 nt, synthetic construct
  source 1..24: mol_type = other RNA
ctatctgcactagatgcaccctta

SEQ ID NO: 359 - RNA, 24 nt, synthetic construct
  source 1..24: mol_type = other RNA
ctatctgcactagatgcatcctta

SEQ ID NO: 360 - RNA, 22 nt, synthetic construct
  source 1..22: mol_type = other RNA
ctatctgcactactgcacctta

SEQ ID NO: 361 - RNA, 23 nt, synthetic construct
  source 1..23: mol_type = other RNA
ctatctgcactactgcaacctta

SEQ ID NO: 362 - RNA, 23 nt, synthetic construct
  source 1..23: mol_type = other RNA
ctatctgcactactgcagcctta

SEQ ID NO: 363 - RNA, 23 nt, synthetic construct
  source 1..23: mol_type = other RNA
ctatctgcactactgcaccctta

SEQ ID NO: 364 - RNA, 23 nt, synthetic construct
  source 1..23: mol_type = other RNA
ctatctgcactactgcatcctta

SEQ ID NO: 365 - RNA, 23 nt, synthetic construct
  source 1..23: mol_type = other RNA
ctatctgcactagatgcacctaa

SEQ ID NO: 366 - RNA, 23 nt, synthetic construct
  source 1..23: mol_type = other RNA
ctatctgcactagatgcaccata

SEQ ID NO: 367 - RNA, 23 nt, synthetic construct
  source 1..23: mol_type = other RNA
ctatctgcactagatgcacgtta

SEQ ID NO: 368 - RNA, 23 nt, synthetic construct
  source 1..23: mol_type = other RNA
ctatctgcactagatgcagctta

SEQ ID NO: 369 - RNA, 23 nt, synthetic construct
  source 1..23: mol_type = other RNA
ctatctgcactagatgctcctta

SEQ ID NO: 370 - RNA, 23 nt, synthetic construct
  source 1..23: mol_type = other RNA
ctatctgcactagatggacctta

SEQ ID NO: 371 - RNA, 23 nt, synthetic construct
  source 1..23: mol_type = other RNA
ctatctgcactagatccacctta

SEQ ID NO: 372 - RNA, 22 nt, synthetic construct
  source 1..22: mol_type = other RNA
ctatctgcactactgcacctaa

SEQ ID NO: 373 - RNA, 22 nt, synthetic construct
  source 1..22: mol_type = other RNA
ctatctgcactactgcaccata

SEQ ID NO: 374 - RNA, 22 nt, synthetic construct
  source 1..22: mol_type = other RNA
ctatctgcactactgcacgtta

SEQ ID NO: 375 - RNA, 22 nt, synthetic construct
  source 1..22: mol_type = other RNA
ctatctgcactactgcagctta

SEQ ID NO: 376 - RNA, 22 nt, synthetic construct
  source 1..22: mol_type = other RNA
ctatctgcactactgctcctta

SEQ ID NO: 377 - RNA, 22 nt, synthetic construct
  source 1..22: mol_type = other RNA
ctatctgcactactggacctta

SEQ ID NO: 378 - RNA, 22 nt, synthetic construct
  source 1..22: mol_type = other RNA
ctatctgcactactccacctta

SEQ ID NO: 379 - RNA, 22 nt, synthetic construct
  source 1..22: mol_type = other RNA
cgaccatggctgtagactgtta

SEQ ID NO: 380 - RNA, 23 nt, synthetic construct
  source 1..23: mol_type = other RNA
cgaccatggctgtagacatgtta

SEQ ID NO: 381 - RNA, 23 nt, synthetic construct
  source 1..23: mol_type = other RNA
cgaccatggctgtagacgtgtta

SEQ ID NO: 382 - RNA, 23 nt, synthetic construct
  source 1..23: mol_type = other RNA
cgaccatggctgtagacctgtta

SEQ ID NO: 383 - RNA, 23 nt, synthetic construct
  source 1..23: mol_type = other RNA
cgaccatggctgtagacttgtta

SEQ ID NO: 384 - RNA, 21 nt, synthetic construct
  source 1..21: mol_type = other RNA
cgaccatggctcagactgtta

SEQ ID NO: 385 - RNA, 22 nt, synthetic construct
  source 1..22: mol_type = other RNA
cgaccatggctcagacatgtta

SEQ ID NO: 386 - RNA, 22 nt, synthetic construct
  source 1..22: mol_type = other RNA
cgaccatggctcagacgtgtta

SEQ ID NO: 387 - RNA, 22 nt, synthetic construct
  source 1..22: mol_type = other RNA
cgaccatggctcagacctgtta

SEQ ID NO: 388 - RNA, 22 nt, synthetic construct
  source 1..22: mol_type = other RNA
cgaccatggctcagacttgtta

SEQ ID NO: 389 - RNA, 22 nt, synthetic construct
  source 1..22: mol_type = other RNA
cgaccatggctgtagactgtaa

SEQ ID NO: 390 - RNA, 22 nt, synthetic construct
  source 1..22: mol_type = other RNA
cgaccatggctgtagactgata

SEQ ID NO: 391 - RNA, 22 nt, synthetic construct
  source 1..22: mol_type = other RNA
cgaccatggctgtagactctta

SEQ ID NO: 392 - RNA, 22 nt, synthetic construct
  source 1..22: mol_type = other RNA
cgaccatggctgtagacagtta

SEQ ID NO: 393 - RNA, 22 nt, synthetic construct
  source 1..22: mol_type = other RNA
cgaccatggctgtagagtgtta

SEQ ID NO: 394 - RNA, 22 nt, synthetic construct
  source 1..22: mol_type = other RNA
cgaccatggctgtagtctgtta

SEQ ID NO: 395 - RNA, 22 nt, synthetic construct
  source 1..22: mol_type = other RNA
cgaccatggctgtacactgtta

SEQ ID NO: 396 - RNA, 21 nt, synthetic construct
  source 1..21: mol_type = other RNA
cgaccatggctcagactgtaa

SEQ ID NO: 397 - RNA, 21 nt, synthetic construct
  source 1..21: mol_type = other RNA
cgaccatggctcagactgata

SEQ ID NO: 398 - RNA, 21 nt, synthetic construct
  source 1..21: mol_type = other RNA
cgaccatggctcagactctta

SEQ ID NO: 399 - RNA, 21 nt, synthetic construct
  source 1..21: mol_type = other RNA
cgaccatggctcagacagtta

SEQ ID NO: 400 - RNA, 21 nt, synthetic construct
  source 1..21: mol_type = other RNA
cgaccatggctcagagtgtta

SEQ ID NO: 401 - RNA, 21 nt, synthetic construct
  source 1..21: mol_type = other RNA
cgaccatggctcagtctgtta

SEQ ID NO: 402 - RNA, 21 nt, synthetic construct
  source 1..21: mol_type = other RNA
cgaccatggctcacactgtta

SEQ ID NO: 403 - RNA, 24 nt, synthetic construct
  source 1..24: mol_type = other RNA
aacccctatcacgattagcattaa

SEQ ID NO: 404 - RNA, 25 nt, synthetic construct
  source 1..25: mol_type = other RNA
aacccctatcacgattagcaattaa

SEQ ID NO: 405 - RNA, 25 nt, synthetic construct
  source 1..25: mol_type = other RNA
aacccctatcacgattagcgattaa

SEQ ID NO: 406 - RNA, 25 nt, synthetic construct
  source 1..25: mol_type = other RNA
aacccctatcacgattagccattaa

SEQ ID NO: 407 - RNA, 25 nt, synthetic construct
  source 1..25: mol_type = other RNA
aacccctatcacgattagctattaa

SEQ ID NO: 408 - RNA, 23 nt, synthetic construct
  source 1..23: mol_type = other RNA
aacccctatcacgtaagcattaa

SEQ ID NO: 409 - RNA, 24 nt, synthetic construct
  source 1..24: mol_type = other RNA
aacccctatcacgtaagcaattaa

SEQ ID NO: 410 - RNA, 24 nt, synthetic construct
  source 1..24: mol_type = other RNA
aacccctatcacgtaagcgattaa

SEQ ID NO: 411 - RNA, 24 nt, synthetic construct
  source 1..24: mol_type = other RNA
aacccctatcacgtaagccattaa

SEQ ID NO: 412 - RNA, 24 nt, synthetic construct
  source 1..24: mol_type = other RNA
aacccctatcacgtaagctattaa

SEQ ID NO: 413 - RNA, 24 nt, synthetic construct
  source 1..24: mol_type = other RNA
aacccctatcacgattagcattta

SEQ ID NO: 414 - RNA, 24 nt, synthetic construct
  source 1..24: mol_type = other RNA
aacccctatcacgattagcataaa

SEQ ID NO: 415 - RNA, 24 nt, synthetic construct
  source 1..24: mol_type = other RNA
aacccctatcacgattagcaataa

SEQ ID NO: 416 - RNA, 24 nt, synthetic construct
  source 1..24: mol_type = other RNA
aacccctatcacgattagctttaa

SEQ ID NO: 417 - RNA, 24 nt, synthetic construct
  source 1..24: mol_type = other RNA
aacccctatcacgattaggattaa

SEQ ID NO: 418 - RNA, 24 nt, synthetic construct
  source 1..24: mol_type = other RNA
aacccctatcacgattaccattaa

SEQ ID NO: 419 - RNA, 24 nt, synthetic construct
  source 1..24: mol_type = other RNA
aacccctatcacgatttgcattaa

SEQ ID NO: 420 - RNA, 23 nt, synthetic construct
  source 1..23: mol_type = other RNA
aacccctatcacgtaagcattta

SEQ ID NO: 421 - RNA, 23 nt, synthetic construct
  source 1..23: mol_type = other RNA
aacccctatcacgtaagcataaa

SEQ ID NO: 422 - RNA, 23 nt, synthetic construct
  source 1..23: mol_type = other RNA
aacccctatcacgtaagcaataa

SEQ ID NO: 423 - RNA, 23 nt, synthetic construct
  source 1..23: mol_type = other RNA
aacccctatcacgtaagctttaa

SEQ ID NO: 424 - RNA, 23 nt, synthetic construct
  source 1..23: mol_type = other RNA
aacccctatcacgtaaggattaa

SEQ ID NO: 425 - RNA, 23 nt, synthetic construct
  source 1..23: mol_type = other RNA
aacccctatcacgtaaccattaa

SEQ ID NO: 426 - RNA, 23 nt, synthetic construct
  source 1..23: mol_type = other RNA
aacccctatcacgtatgcattaa

SEQ ID NO: 427 - RNA, 76 nt, synthetic construct
  source 1..76: mol_type = other RNA
tgatatacgaccagtgcgcactacacaacccctatcacgtaagcattaagaaactcaagatactggtcgtatatca

SEQ ID NO: 428 - RNA, 76 nt, synthetic construct
  source 1..76: mol_type = other RNA
tgataagaaatgacagcgcactacacaacccctatcacgtaagcattaagaaactcaagattgtcatttcttatca

SEQ ID NO: 429 - RNA, 76 nt, synthetic construct
  source 1..76: mol_type = other RNA
cattagtatgttaaggcgcactacacaacccctatcacgtaagcattaagaaactcaagatcttaacatactaatg

SEQ ID NO: 430 - RNA, 76 nt, synthetic construct
  source 1..76: mol_type = other RNA
tcaatacagattattgcgcactacacaacccctatcacgtaagcattaagaaactcaagataataatctgtattga

SEQ ID NO: 431 - RNA, 76 nt, synthetic construct
  source 1..76: mol_type = other RNA
ttatatacttcaagggcgcactacacaacccctatcacgtaagcattaagaaactcaagatccttgaagtatataa

SEQ ID NO: 432 - RNA, 76 nt, synthetic construct
  source 1..76: mol_type = other RNA
ttgtttattgacattgcgcactacacaacccctatcacgtaagcattaagaaactcaagataatgtcaataaacaa

SEQ ID NO: 433 - RNA, 84 nt, synthetic construct
  source 1..84: mol_type = other RNA
gggtgatatacgaccagtatagactacaacgaccatggctcagactgttagaaactcaagatactggtctatatcattatgagc

SEQ ID NO: 434 - RNA, 165 nt, synthetic construct
  source 1..165: mol_type = other RNA
gggtgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagaca

SEQ ID NO: 435 - RNA, 246 nt, synthetic construct
  source 1..246: mol_type = other RNA
gggtgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagacacattagtatgttaagctcatccttatcgaccatggctcagactgttacactcaaatcatcttaacaactaatgtttagtag

SEQ ID NO: 436 - RNA, 327 nt, synthetic construct
  source 1..327: mol_type = other RNA
gggtgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagacacattagtatgttaagctcatccttatcgaccatggctcagactgttacactcaaatcatcttaacaactaatgtttagtagtcaatacagattatgactctatcaaacgaccatggctcagactgttagcaacatcatcacataatcgtattgacttgcaat

SEQ ID NO: 437 - RNA, 408 nt, synthetic construct
  source 1..408: mol_type = other RNA
gggtgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagacacattagtatgttaagctcatccttatcgaccatggctcagactgttacactcaaatcatcttaacaactaatgtttagtagtcaatacagattatgactctatcaaacgaccatggctcagactgttagcaacatcatcacataatcgtattgacttgcaatctatatacttcaaggaagatcactaacgaccatggctcagactgttacactactaccaaccttgaatatatagccgtagtt

SEQ ID NO: 438 - RNA, 489 nt, synthetic construct
  source 1..489: mol_type = other RNA
gggtgatatacgaccagtatagactacaacgaccatggctcagactgttagaaactcaagatactggtctatatcattatgagctgataagaaatgactcaacttacgtacgaccatggctcagactgttacaatatcacctcagtcattcttatcacttagacacattagtatgttaagctcatccttatcgaccatggctcagactgttacactcaaatcatcttaacaactaatgtttagtagtcaatacagattatgactctatcaaacgaccatggctcagactgttagcaacatcatcacataatcgtattgacttgcaatctatatacttcaaggaagatcactaacgaccatggctcagactgttacactactaccaaccttgaatatatagccgtagttctgtttattgacatgtcaacatcaatcgaccatggctcagactgttacactttacctaacatgtcataaacagtaacagaa

SEQ ID NO: 439 - RNA, 228 nt, synthetic construct
  source 1..228: mol_type = other RNA
gggtgatatacgaccagtattacattatacgaccatggctcagactgttacttacccactcaactggtctatatcatgtgataagaaatgactctccactttcccgaccatggctcagactgttaaaactcttataaagtcattcttatcaaacattagtatgttaagtaaattcattacgaccatggctcagactgttaccaattcacctccttaacaactaatgcc

SEQ ID NO: 440 - RNA, 234 nt, synthetic construct
  source 1..234: mol_type = other RNA
gggtgatatacgaccagtatttacatattcgaccatggctcagactgttactaattcacaaaactggtctatatcataattgataagaaatgactataccaaatctcgaccatggctcagactgttaaaatcataccaaagtcattcttatcacattcattagtatgttaagctcacacctctcgaccatggctcagactgttaaaatcctaatcacttaacaactaatgtatt

SEQ ID NO: 441 - RNA, 240 nt, synthetic construct
  source 1..240: mol_type = other RNA
gggtgatatacgaccagttcctttatacacgaccatggctcagactgttaacatattctttcactggtctatatcatcattttgataagaaatgactcacgaacaccccgaccatggctcagactgttacctactcctattagtcattcttatcatccacacattagtatgttaagtattcccaacccgaccatggctcagactgttaacaccaatttcccttaacaactaatgcttctc

SEQ ID NO: 442 - RNA, 246 nt, synthetic construct
  source 1..246: mol_type = other RNA
gggtgatatacgaccagtttctcacacttcgaccatggctcagactgttaataccaatctatactggtctatatcacttatacttgataagaaatgactctccacttaaacgaccatggctcagactgttacaactatcctttagtcattcttatcatacctatccattagtatgttaagtaaacctacttcgaccatggctcagactgttaactccttctcatcttaacaactaatgaacaaact

SEQ ID NO: 443 - RNA, 453 nt, synthetic construct
  source 1..453: mol_type = other RNA
gggtgatatacgaccagtattacattatacgaccatggctcagactgttacttacccactcaactggtctatatcatgtgataagaaatgactctccactttcccgaccatggctcagactgttaaaactcttataaagtcattcttatcaaacattagtatgttaagtaaattcattacgaccatggctcagactgttaccaattcacctccttaacaactaatgcctcaatacagattatgtatctcacgtacgaccatggctcagactgttaataactccacctcataatcgtattgactctatatacttcaaggctaccaaacaacgaccatggctcagactgttaacaattaccacaccttgaatatatagccctgtttattgacatgaccctccgcaacgaccatggctcagactgttaccaccttactcacatgtcataaacagcc

SEQ ID NO: 444 - RNA, 465 nt, synthetic construct
  source 1..465: mol_type = other RNA
gggtgatatacgaccagtatttacatattcgaccatggctcagactgttactaattcacaaaactggtctatatcataattgataagaaatgactataccaaatctcgaccatggctcagactgttaaaatcataccaaagtcattcttatcacattcattagtatgttaagctcacacctctcgaccatggctcagactgttaaaatcctaatcacttaacaactaatgtatttcaatacagattatgcaaattaagaacgaccatggctcagactgttacaataccacgcacataatcgtattgatcctctatatacttcaaggtaatccctatccgaccatggctcagactgttacttatccacttcccttgaatatatagcctactgtttattgacatgccctccaccgacgaccatggctcagactgttaccaaccctatcacatgtcataaacagataa

SEQ ID NO: 445 - RNA, 477 nt, synthetic construct
  source 1..477: mol_type = other RNA
gggtgatatacgaccagttcctttatacacgaccatggctcagactgttaacatattctttcactggtctatatcatcattttgataagaaatgactcacgaacaccccgaccatggctcagactgttacctactcctattagtcattcttatcatccacacattagtatgttaagtattcccaacccgaccatggctcagactgttaacaccaatttcccttaacaactaatgcttctctcaatacagattatgtactatatgctcgaccatggctcagactgttacacttaccaaaccataatcgtattgaccaacactatatacttcaaggcgaataacaaacgaccatggctcagactgttaaacctttacaatccttgaatatatagctatcactgtttattgacatgcttaacccgaacgaccatggctcagactgttacaaccaatactacatgtcataaacagcacccg

SEQ ID NO: 446 - RNA, 489 nt, synthetic construct
  source 1..489: mol_type = other RNA
gggtgatatacgaccagtttctcacacttcgaccatggctcagactgttaataccaatctatactggtctatatcacttatacttgataagaaatgactctccacttaaacgaccatggctcagactgttacaactatcctttagtcattcttatcatacctatccattagtatgttaagtaaacctacttcgaccatggctcagactgttaactccttctcatcttaacaactaatgaacaaacttcaatacagattatgcaacgcaaacccgaccatggctcagactgttaaaccaatacacccataatcgtattgacgaaactactatatacttcaaggctcttcacacccgaccatggctcagactgttaaaatcctctatcccttgaatatatagaacctctactgtttattgacatgcaatatttaatcgaccatggctcagactgttaaacttccaacaccatgtcataaacagcgcgcctc

SEQ ID NO: 447 - RNA, 231 nt, synthetic construct
  source 1..231: mol_type = other RNA
gggtcaatacagatttcactttcactcgaccatggctcagactgttacataacctaatcaatctgtattgaaacattacctatatacttcagctccacatatcgaccatggctcagactgttaccctccaatcaatgaagtatatagccattcacctgtttattgaccaccttcaacacgaccatggctcagactgttacctcatatgcacgtcaataaacagttcacctg

SEQ ID NO: 448 - RNA, 228 nt, synthetic construct
  source 1..228: mol_type = other RNA
gggtctgcattgctacccaaccaacacgaccatggctcagactgttaactttcctcaattagcaagcagacaaactcactcttcgaaccatcactaaattccgaccatggctcagactgttacatatcactatttggttcaagagaactccaagccacagcaacagcgtatttaaccgaccatggctcagactgttactttcctcataatgttgcgtggccccaatta

SEQ ID NO: 449 - RNA, 231 nt, synthetic construct
  source 1..231: mol_type = other RNA
gggccgataccatcaatgcaacttcacgaccatggctcagactgttaaacacaactatatgatggaatcggtacctaactctacttcagagcactatacaaacgaccatggctcagactgttaaacatttaccacctctgatgtagatcatacaggcatatacaacgctccaaccgaacgaccatggctcagactgttacctaacctacaccgttgtttatgcgaatctca

SEQ ID NO: 450 - RNA, 249 nt, synthetic construct
  source 1..249: mol_type = other RNA
gggtgatatacgaccagtttattctccgacgaccatggctcagactgttaagactcctacccactggtcgtatatcacccgtctttgataagaaatgactcccttccttatcgaccatggctcagactgttactatacctttccagtcatttcttatcacaactttacattagtatgttaagtgcacctaattcgaccatggctcagactgttaacactacctattcttaacatactaatgatttacaa

SEQ ID NO: 451 - RNA, 246 nt, synthetic construct
  source 1..246: mol_type = other RNA
gggtgatatacgaccagtaaaccaaccaccgaccatggctcagactgttacaatttccatacactggtctatatcataatttattgataagaaatgactctccttctactcgaccatggctcagactgttaaccacttctattagtcattcttatcattctaacccattagtatgttaagtcataccaacccgaccatggctcagactgttacctatatttactcttaacaactaatgacttcact

SEQ ID NO: 452 - RNA, 249 nt, synthetic construct
  source 1..249: mol_type = other RNA
gggtgatatacgaccagttatacattaatcgaccatggctcagactgttaaacactacccacactggtcctatatcatcatataatgataagaaatgactctttcctcaaccgaccatggctcagactgttacacctattcataagtcattacttatcacacctattcattagtatgttaagtattcttatcacgaccatggctcagactgttactaaatctaacccttaacaaactaatgcactatgc

SEQ ID NO: 453 - RNA, 459 nt, synthetic construct
  source 1..459: mol_type = other RNA
gggtatacgaccagtcaacataacgacgaccatggctcagactgttagcacctccataaactggtcgtatataattgcctgataagaaatgctctataatcacgaccatggctcagactgttaattaatctctaacatttcttatcaccctacgacattagtatgtttctctttctaacgaccatggctcagactgttactaacacatattaacatactaatgccttaatatcaatacagatttcactttcactcgaccatggctcagactgttacataacctaatcaatctgtattgaaacattacctatatacttcagctccacatatcgaccatggctcagactgttaccctccaatcaatgaagtatatagccattcacctgtttattgaccaccttcaacacgaccatggctcagactgttacctcatatgcacgtcaataaacagttcacctg

SEQ ID NO: 454 - RNA, 453 nt, synthetic construct
  source 1..453: mol_type = other RNA
ggggtcagatacatccctatacgaaacgaccatggctcagactgttaatttaatgcaccgatgtactgacccctataaccagctcttgcatttccaatacgcgaccatggctcagactgttacaaccctccatttgcaaggctggccctcaactgccaaatgcctacctataccttcgaccatggctcagactgttaataattccctcgaggcattggcactttctcaccgtatagcccttacacaaatctcgaccatggctcagactgttaaatctctatcacagggcttacgggtcccgaactttgtcagcatccacaccttatcgaccatggctcagactgttaaattaatatttcatgctgcaaagattaccgctcagcactcttcactaatcctatcgaccatggctcagactgttaacatcaccgcacgaagaggctgatcttaaag

SEQ ID NO: 455 - RNA, 459 nt, synthetic construct
  source 1..459: mol_type = other RNA
gggtctcgtcactctttcctctcctacgaccatggctcagactgttaaccacttatcttagagtgtcgagaaaccatacacctctaccgcaaacttacttcacgaccatggctcagactgttaacctttcttatctgcggttgaggttacttgactcacgttcttgcccaatactccccgaccatggctcagactgttacccaattccactgcaagatcgtgactatccaaaagcaccagatccattacaaatacgaccatggctcagactgttaacaatactgcaagatctgctgcttcaaatacctaaatatgcttcgataaactacacgaccatggctcagactgttacccttcattgcagaagcaaatttaatcttaactcacaagctcaagaccgataccacgaccatggctcagactgttacatccttactaattgagcatgtgaaatccctt

SEQ ID NO: 456 - RNA, 489 nt, synthetic construct
  source 1..489: mol_type = other RNA
gggtgatatacgaccagtaaaccaaccaccgaccatggctcagactgttacaatttccatacactggtctatatcataatttattgataagaaatgactctccttctactcgaccatggctcagactgttaaccacttctattagtcattcttatcattctaacccattagtatgttaagtcataccaacccgaccatggctcagactgttacctatatttactcttaacaactaatgacttcacttcaatacagattatgcacctaaatcacgaccatggctcagactgttacttatacacttccataatcgtattgactaacttcctatatacttcaaggcgactacactacgaccatggctcagactgttaccacacgatttaccttgaatatatagacacctgactgtttattgacatgaatctacaatacgaccatggctcagactgttaccaaccacaccacatgtcataaacagcctttctg

SEQ ID NO: 457 - RNA, 495 nt, synthetic construct
  source 1..495: mol_type = other RNA
gggtgatatacgaccagttatacattaatcgaccatggctcagactgttaaacactacccacactggtcctatatcatcatataatgataagaaatgactctttcctcaaccgaccatggctcagactgttacacctattcataagtcattacttatcacacctattcattagtatgttaagtattcttatcacgaccatggctcagactgttactaaatctaacccttaacaaactaatgcactatgctcaatacagattatgcattcaaatcacgaccatggctcagactgttaaagaaattacaacataatcagtattgactacattcctatatacttcaaggaatctttctcacgaccatggctcagactgttaacttacctttcaccttgaactatatagctaaccaactgtttattgacatgatctatacgcacgaccatggctcagactgttacattatcacctacatgtcattaaacagaattctgt

SEQ ID NO: 458 - RNA, 249 nt, synthetic construct
  source 1..249: mol_type = other RNA
gggtgatatacgaccagttatcaaattaacgaccatggctgtagactgttagacaatcaaatcactggtctatatcaccaaactttgataagaaatgactttactaatcaacgaccatggctgtagactgttagacacatattctagtcattcttatcaccgaataccattagtatgttaagtataaaccgaacgaccatggctgtagactgttaaacctctcaaatcttaacaactaatgatgcaact

SEQ ID NO: 459 - RNA, 252 nt, synthetic construct
  source 1..252: mol_type = other RNA
gggtgatatacgaccagttcacaccgtaccgaccatggctgtagacatgttaactgacaattatactggtctatatcatcgactcgtgataagaaatgactgttcaatgtaccgaccatggctgtagacatgttaccaccgctttagagtcattcttatcagctcaatccattagtatgttaagctttcaatatccgaccatggctgtagacatgttattattccgtattcttaacaactaatgatttccta

SEQ ID NO: 460 - RNA, 252 nt, synthetic construct
  source 1..252: mol_type = other RNA
gggtgatatacgaccagttacgcacctttcgaccatggctgtagacgtgttagttccgaacattactggtctatatcaccgactcctgataagaaatgactcctttccttaacgaccatggctgtagacgtgttatgccgcttattcagtcattcttatcaacttcacccattagtatgttaagaataacccgcccgaccatggctgtagacgtgttatgacaatttcagcttaacaactaatgattatcca

SEQ ID NO: 461 - RNA, 252 nt, synthetic construct
  source 1..252: mol_type = other RNA
gggtgatatacgaccagttcccacgatctcgaccatggctgtagacctgttaatctgaactcctactggtctatatcaccaacgactgataagaaatgactgaatcacaatgcgaccatggctgtagacctgttataaacctcttaaagtcattcttatcacacccaaccattagtatgttaagaaccatatccccgaccatggctgtagacctgttacctcaattatcgcttaacaactaatgacaacgcg

SEQ ID NO: 462 - RNA, 252 nt, synthetic construct
  source 1..252: mol_type = other RNA
gggtgatatacgaccagtcctatttaattcgaccatggctgtagacttgttaaacccttccaacactggtctatatcaaccctatctgataagaaatgactatccactaacacgaccatggctgtagacttgttaatattacttatcagtcattcttatcaacgccgaccattagtatgttaagtccaaataacccgaccatggctgtagacttgttaaaccctccgcttcttaacaactaatgattactta

SEQ ID NO: 463 - RNA, 249 nt, synthetic construct
  source 1..249: mol_type = other RNA
gggtgatatacgaccagtactaagaatatcgaccatggctgtagactgtaatacttaatttaaactggtctatatcattatgagctgataagaaatgactataaattatcacgaccatggctgtagactgtaactaatcaactaaagtcattcttatcattcagacacattagtatgttaagactaattatctcgaccatggctgtagactgtaactaatccttcaccttaacaactaatgtttagtag

SEQ ID NO: 464 - RNA, 249 nt, synthetic construct
  source 1..249: mol_type = other RNA
gggtgatatacgaccagtagagaacataacgaccatggctgtagactgataataacgtaccgaactggtctatatcattatgagctgataagaaatgactgtataatttcccgaccatggctgtagactgataacttaccacataagtcattcttatcattcagacacattagtatgttaagttccaactcttcgaccatggctgtagactgataatccgactcttccttaacaactaatgtttagtag

SEQ ID NO: 465 - RNA, 249 nt, synthetic construct
  source 1..249: mol_type = other RNA
gggtgatatacgaccagtaacacattaatcgaccatggctgtagactcttatccttacttaaaactggtctatatcattatgagctgataagaaatgacttcaaagtatatcgaccatggctgtagactcttaatccctaatcctagtcattcttatcattcagacacattagtatgttaagcttatcctagacgaccatggctgtagactcttaatatcaaatcaacttaacaactaatgtttagtag

SEQ ID NO: 466 - RNA, 249 nt, synthetic construct
  source 1..249: mol_type = other RNA
gggtgatatacgaccagtacacaacgttccgaccatggctgtagacagttaatataattcaagactggtctatatcattatgagctgataagaaatgacttgaacacctaccgaccatggctgtagacagttaaacctttataatagtcattcttatcattcagacacattagtatgttaagaagacatttaccgaccatggctgtagacagttaaacaatcacatacttaacaactaatgtttagtag

SEQ ID NO: 467 - RNA, 249 nt, synthetic construct
  source 1..249: mol_type = other RNA
gggtgatatacgaccagtcatcaactaatcgaccatggctgtagagtgttaatatctcaattcactggtctatatcattatgagctgataagaaatgactctccctcacgacgaccatggctgtagagtgttagatcaattcataagtcattcttatcattcagacacattagtatgttaagttaattcaaaccgaccatggctgtagagtgttacctccctcaattcttaacaactaatgtttagtag

SEQ ID NO: 468 - RNA, 249 nt, synthetic construct
  source 1..249: mol_type = other RNA
gggtgatatacgaccagtgatataactttcgaccatggctgtagtctgttaacacccgctcaaactggtctatatcattatgagctgataagaaatgactttcaataagtacgaccatggctgtagtctgttattcacaccctctagtcattcttatcattcagacacattagtatgttaagctccaccgtcgcgaccatggctgtagtctgttatcatatgtctttcttaacaactaatgtttagtag

SEQ ID NO: 469 - RNA, 249 nt, synthetic construct
  source 1..249: mol_type = other RNA
gggtgatatacgaccagtcattaatcctccgaccatggctgtacactgttattaccaaacactactggtctatatcattatgagctgataagaaatgactctctcgcttatcgaccatggctgtacactgttacctaacctaatcagtcattcttatcattcagacacattagtatgttaagtcctaatatcgcgaccatggctgtacactgttaataaagtcaattcttaacaactaatgtttagtag

SEQ ID NO: 470 - RNA, 249 nt, synthetic construct
  source 1..249: mol_type = other RNA
gggtgatatacgaccagtgtgctccacctcgaccatggctcagacatgttacatactttgatgactggtctatatcaccacaatttgataagaaatgactattaagattaccgaccatggctcagacatgttaaaatagatatcgagtcattcttatcaccagatcccattagtatgttaagaaaccgttctgcgaccatggctcagacatgttaaaataagatatgcttaacaactaatgcagtagca

SEQ ID NO: 471 - RNA, 249 nt, synthetic construct
  source 1..249: mol_type = other RNA
gggtgatatacgaccagttcctcagtaaacgaccatggctcagacgtgttatattctaatcccactggtctatatcattcacacttgataagaaatgactagaaattgactcgaccatggctcagacgtgttaaaatattgaataagtcattcttatcataaatccacattagtatgttaagaatgaacccaccgaccatggctcagacgtgttaaaatattgtgaacttaacaactaatggaatgact

SEQ ID NO: 472 - RNA, 249 nt, synthetic construct
  source 1..249: mol_type = other RNA
gggtgatatacgaccagttcactcttaaacgaccatggctcagacctgttaaaaccttacattactggtctatatcaataatgtttgataagaaatgacttgatatctacccgaccatggctcagacctgttaaaatgctcctatagtcattcttatcatttactctcattagtatgttaagagactagaatacgaccatggctcagacctgttaaaatatccacaacttaacaactaatgtatgaacc

SEQ ID NO: 473 - RNA, 249 nt, synthetic construct
  source 1..249: mol_type = other RNA
gggtgatatacgaccagttgcacgcactccgaccatggctcagacttgttaaaattatacactactggtctatatcacccttacttgataagaaatgactctaaatgcactcgaccatggctcagacttgttaaatattctacacagtcattcttatcattcgtctccattagtatgttaagatctctatcttcgaccatggctcagacttgttacacttaaattcgcttaacaactaatgtctcaacc

SEQ ID NO: 474 - RNA, 246 nt, synthetic construct
  source 1..246: mol_type = other RNA
gggtgatatacgaccagtcaaccaactatcgaccatggctcagactgtaacatttaactcaaactggtctatatcaccactccctgataagaaatgactatatactaattcgaccatggctcagactgtaatataagacatcaagtcattcttatcaaaactatccattagtatgttaagtgacacgcacccgaccatggctcagactgtaaattccaaatcctcttaacaactaatgcattccaa

SEQ ID NO: 475 - RNA, 246 nt, synthetic construct
  source 1..246: mol_type = other RNA
gggtgatatacgaccagtaatacacttcacgaccatggctcagactgatattactataaaccactggtctatatcactacaatatgataagaaatgacttttccacactacgaccatggctcagactgatataaatctaacatagtcattcttatcaagcaaccacattagtatgttaagatacataattacgaccatggctcagactgataaccttccttccccttaacaactaatgctttaata

SEQ ID NO: 476 - RNA, 246 nt, synthetic construct
  source 1..246: mol_type = other RNA
gggtgatatacgaccagttcagacaaacacgaccatggctcagactcttactatctcaacacactggtctatatcacatttacatgataagaaatgacttgaacaaacgacgaccatggctcagactcttactaaattctcttagtcattcttatcacatctactcattagtatgttaaggaacagcattccgaccatggctcagactcttagatacagtaccacttaacaactaatgcatcaata

SEQ ID NO: 477 - RNA, 246 nt, synthetic construct
  source 1..246: mol_type = other RNA
gggtgatatacgaccagttatacagactacgaccatggctcagacagttacaaacgcgaaccactggtctatatcacacaattttgataagaaatgactttacaaatatccgaccatggctcagacagttaaaaccgcaacatagtcattcttatcatcccgcatcattagtatgttaagctacaaatcaccgaccatggctcagacagttagacaccttccctcttaacaactaatgttctacac

SEQ ID NO: 478 - RNA, 246 nt, synthetic construct
  source 1..246: mol_type = other RNA
gggtgatatacgaccagttttctatccttcgaccatggctcagagtgttaatactatatcatactggtctatatcatctatctttgataagaaatgactatcctacccgacgaccatggctcagagtgttatatccattgacgagtcattcttatcattaccaaccattagtatgttaagaaatcttccaccgaccatggctcagagtgttaaaccatctacagcttaacaactaatgtgctcatc

SEQ ID NO: 479 - RNA, 246 nt, synthetic construct
  source 1..246: mol_type = other RNA
gggtgatatacgaccagtctgcgaatacccgaccatggctcagtctgttacctttgatcttaactggtctatatcatctacctctgataagaaatgactgtgaacattcccgaccatggctcagtctgttatctaacgaaataagtcattcttatcaaactcaaccattagtatgttaagagatacttactcgaccatggctcagtctgttatcctaccttccgcttaacaactaatgtactcatc

SEQ ID NO: 480 - RNA, 246 nt, synthetic construct
  source 1..246: mol_type = other RNA
gggtgatatacgaccagtaccctcatctacgaccatggctcacactgttattcttatcattaactggtctatatcattccaaattgataagaaatgactctgactattcgcgaccatggctcacactgttaaatatgaaacctagtcattcttatcacactcaaccattagtatgttaaggcaatctacttcgaccatggctcacactgttatacaacctacgacttaacaactaatgaagctcag

SEQ ID NO: 481 - RNA, 495 nt, synthetic construct
  source 1..495: mol_type = other RNA
gggtgatatacgaccagttatcaaattaacgaccatggctgtagactgttagacaatcaaatcactggtctatatcaccaaactttgataagaaatgactttactaatcaacgaccatggctgtagactgttagacacatattctagtcattcttatcaccgaataccattagtatgttaagtataaaccgaacgaccatggctgtagactgttaaacctctcaaatcttaacaactaatgatgcaacttcaatacagattatgatacaaattcacgaccatggctgtagactgttagattacctttagcataatcgtattgactctgaacctatatacttcaaggctctaatctttcgaccatggctgtagactgttaaaatgtaccaatccttgaatatatagaagatttactgtttattgacatgtactcataatccgaccatggctgtagactgttaataacccaacctcatgtcataaacagaaacataa

SEQ ID NO: 482 - RNA, 501 nt, synthetic construct
  source 1..501: mol_type = other RNA
gggtgatatacgaccagttcacaccgtaccgaccatggctgtagacatgttaactgacaattatactggtctatatcatcgactcgtgataagaaatgactgttcaatgtaccgaccatggctgtagacatgttaccaccgctttagagtcattcttatcagctcaatccattagtatgttaagctttcaatatccgaccatggctgtagacatgttattattccgtattcttaacaactaatgatttcctatcaatacagattatgagagtcccatacgaccatggctgtagacatgttaagactaatcaagcataatcgtattgaatctgaacctatatacttcaaggtccacgctccacgaccatggctgtagacatgttaactgaatcatacccttgaatatatagctgccacgctgtttattgacatgctaattaaccccgaccatggctgtagacatgttaagcaatacatatcatgtcataaacagatattaag

SEQ ID NO: 483 - RNA, 501 nt, synthetic construct
  source 1..501: mol_type = other RNA
gggtgatatacgaccagttacgcacctttcgaccatggctgtagacgtgttagttccgaacattactggtctatatcaccgactcctgataagaaatgactcctttccttaacgaccatggctgtagacgtgttatgccgcttattcagtcattcttatcaacttcacccattagtatgttaagaataacccgcccgaccatggctgtagacgtgttatgacaatttcagcttaacaactaatgattatccatcaatacagattatgagaacatccctcgaccatggctgtagacgtgttattccaacctaaccataatcgtattgaaattacaactatatacttcaagggactataaactcgaccatggctgtagacgtgttacctattaagataccttgaatatatagcactatacctgtttattgacatgttgctttctcccgaccatggctgtagacgtgttattccttgaactccatgtcataaacagtgcacttt

SEQ ID NO: 484 - RNA, 501 nt, synthetic construct
  source 1..501: mol_type = other RNA
gggtgatatacgaccagttcccacgatctcgaccatggctgtagacctgttaatctgaactcctactggtctatatcaccaacgactgataagaaatgactgaatcacaatgcgaccatggctgtagacctgttataaacctcttaaagtcattcttatcacacccaaccattagtatgttaagaaccatatccccgaccatggctgtagacctgttacctcaattatcgcttaacaactaatgacaacgcgtcaatacagattatgactatcctagacgaccatggctgtagacctgttaattcactcctaacataatcgtattgaatccaatcctatatacttcaaggcataactaacacgaccatggctgtagacctgttaattacaagattaccttgaatatatagtagagcacctgtttattgacatgaatcctcaacacgaccatggctgtagacctgttaagacttaaccaacatgtcataaacagtagaccaa

SEQ ID NO: 485 - RNA, 501 nt, synthetic construct
  source 1..501: mol_type = other RNA
gggtgatatacgaccagtcctatttaattcgaccatggctgtagacttgttaaacccttccaacactggtctatatcaaccctatctgataagaaatgactatccactaacacgaccatggctgtagacttgttaatattacttatcagtcattcttatcaacgccgaccattagtatgttaagtccaaataacccgaccatggctgtagacttgttaaaccctccgcttcttaacaactaatgattacttatcaatacagattatgactccttcacacgaccatggctgtagacttgttaaactccttactgcataatcgtattgaatgaaatactatatacttcaaggttcatattaatcgaccatggctgtagacttgttaccactctcaaatccttgaatatatagatgatctcctgtttattgacatgtgcataaattccgaccatggctgtagacttgttaaattaccaatatcatgtcataaacagatttacaa

SEQ ID NO: 486 - RNA, 495 nt, synthetic construct
  source 1..495: mol_type = other RNA
gggtgatatacgaccagtactaagaatatcgaccatggctgtagactgtaatacttaatttaaactggtctatatcattatgagctgataagaaatgactataaattatcacgaccatggctgtagactgtaactaatcaactaaagtcattcttatcattcagacacattagtatgttaagactaattatctcgaccatggctgtagactgtaactaatccttcaccttaacaactaatgtttagtagtcaatacagattatgtcactcaaacccgaccatggctgtagactgtaattcacttcatatcataatcgtattgattctttcactatatacttcaagggacttgaaacacgaccatggctgtagactgtaaaaataactcttaccttgaatatatagttcatgccctgtttattgacatgtaactttccttcgaccatggctgtagactgtaaatcctcacatatcatgtcataaacagttatacaa

SEQ ID NO: 487 - RNA, 495 nt, synthetic construct
  source 1..495: mol_type = other RNA
gggtgatatacgaccagtagagaacataacgaccatggctgtagactgataataacgtaccgaactggtctatatcattatgagctgataagaaatgactgtataatttcccgaccatggctgtagactgataacttaccacataagtcattcttatcattcagacacattagtatgttaagttccaactcttcgaccatggctgtagactgataatccgactcttccttaacaactaatgtttagtagtcaatacagattatgtgaacctccgacgaccatggctgtagactgataatatcccgcaatcataatcgtattgattctttcactatatacttcaaggataatttaattcgaccatggctgtagactgataatccaataacccccttgaatatatagttcatgccctgtttattgacatgattccctagaacgaccatggctgtagactgataaatattactttccatgtcataaacagttatacaa

SEQ ID NO: 488 - RNA, 495 nt, synthetic construct
  source 1..495: mol_type = other RNA
gggtgatatacgaccagtaacacattaatcgaccatggctgtagactcttatccttacttaaaactggtctatatcattatgagctgataagaaatgacttcaaagtatatcgaccatggctgtagactcttaatccctaatcctagtcattcttatcattcagacacattagtatgttaagcttatcctagacgaccatggctgtagactcttaatatcaaatcaacttaacaactaatgtttagtagtcaatacagattatgcattaactttccgaccatggctgtagactcttatccctcattatacataatcgtattgattctttcactatatacttcaaggattaaccgcatcgaccatggctgtagactcttaaataacccgcaaccttgaatatatagttcatgccctgtttattgacatgtaatccaatttcgaccatggctgtagactcttaattaatccctatcatgtcataaacagttatacaa

SEQ ID NO: 489 - RNA, 495 nt, synthetic construct
  source 1..495: mol_type = other RNA
gggtgatatacgaccagtacacaacgttccgaccatggctgtagacagttaatataattcaagactggtctatatcattatgagctgataagaaatgacttgaacacctaccgaccatggctgtagacagttaaacctttataatagtcattcttatcattcagacacattagtatgttaagaagacatttaccgaccatggctgtagacagttaaacaatcacatacttaacaactaatgtttagtagtcaatacagattatgcccgctcctatcgaccatggctgtagacagttacctatcctaacccataatcgtattgattctttcactatatacttcaaggacattataaatcgaccatggctgtagacagttacttaatctaacaccttgaatatatagttcatgccctgtttattgacatgcttaagacgaacgaccatggctgtagacagttaaattacaactaccatgtcataaacagttatacaa

SEQ ID NO: 490 - RNA, 495 nt, synthetic construct
  source 1..495: mol_type = other RNA
gggtgatatacgaccagtcatcaactaatcgaccatggctgtagagtgttaatatctcaattcactggtctatatcattatgagctgataagaaatgactctccctcacgacgaccatggctgtagagtgttagatcaattcataagtcattcttatcattcagacacattagtatgttaagttaattcaaaccgaccatggctgtagagtgttacctccctcaattcttaacaactaatgtttagtagtcaatacagattatgatatcccacatcgaccatggctgtagagtgttaaatccgtatccccataatcgtattgattctttcactatatacttcaaggctcccttaacccgaccatggctgtagagtgttataaattcctaatccttgaatatatagttcatgccctgtttattgacatgttcccttaatccgaccatggctgtagagtgttaaacaaatctcctcatgtcataaacagttatacaa

SEQ ID NO: 491 - RNA, 495 nt, synthetic construct
  source 1..495: mol_type = other RNA
gggtgatatacgaccagtgatataactttcgaccatggctgtagtctgttaacacccgctcaaactggtctatatcattatgagctgataagaaatgactttcaataagtacgaccatggctgtagtctgttattcacaccctctagtcattcttatcattcagacacattagtatgttaagctccaccgtcgcgaccatggctgtagtctgttatcatatgtctttcttaacaactaatgtttagtagtcaatacagattatgtacattatcttcgaccatggctgtagtctgttaccatacattctccataatcgtattgattctttcactatatacttcaaggcctcatcctatcgaccatggctgtagtctgttaatcccgcgacatccttgaatatatagttcatgccctgtttattgacatgtccaaccttcgcgaccatggctgtagtctgttatctttcaacactcatgtcataaacagttatacaa

SEQ ID NO: 492 - RNA, 495 nt, synthetic construct
  source 1..495: mol_type = other RNA
gggtgatatacgaccagtcattaatcctccgaccatggctgtacactgttattaccaaacactactggtctatatcattatgagctgataagaaatgactctctcgcttatcgaccatggctgtacactgttacctaacctaatcagtcattcttatcattcagacacattagtatgttaagtcctaatatcgcgaccatggctgtacactgttaataaagtcaattcttaacaactaatgtttagtagtcaatacagattatgacttcactcaacgaccatggctgtacactgttatcctaatctccgcataatcgtattgattctttcactatatacttcaaggctcttatcttccgaccatggctgtacactgttaattaagcttctcccttgaatatatagttcatgccctgtttattgacatgcaacccttccacgaccatggctgtacactgttagatactccgaaccatgtcataaacagttatacaa

SEQ ID NO: 493 - RNA, 495 nt, synthetic construct
  source 1..495: mol_type = other RNA
gggtgatatacgaccagtgtgctccacctcgaccatggctcagacatgttacatactttgatgactggtctatatcaccacaatttgataagaaatgactattaagattaccgaccatggctcagacatgttaaaatagatatcgagtcattcttatcaccagatcccattagtatgttaagaaaccgttctgcgaccatggctcagacatgttaaaataagatatgcttaacaactaatgcagtagcatcaatacagattatgcctcaatccttcgaccatggctcagacatgttaaaatataatgctcataatcgtattgaaatttagactatatacttcaaggaaacagtagatcgaccatggctcagacatgttacaccagaaagaaccttgaatatatagccaagaacctgtttattgacatgtacttcaagaacgaccatggctcagacatgttaaaataagcttaccatgtcataaacagaccttatt

SEQ ID NO: 494 - RNA, 495 nt, synthetic construct
  source 1..495: mol_type = other RNA
gggtgatatacgaccagttcctcagtaaacgaccatggctcagacgtgttatattctaatcccactggtctatatcattcacacttgataagaaatgactagaaattgactcgaccatggctcagacgtgttaaaatattgaataagtcattcttatcataaatccacattagtatgttaagaatgaacccaccgaccatggctcagacgtgttaaaatattgtgaacttaacaactaatggaatgacttcaatacagattatgtcaactatctacgaccatggctcagacgtgttaaaatcttattcccataatcgtattgactccacgcctatatacttcaaggacaacttaccacgaccatggctcagacgtgttaaaatattaccacccttgaatatatagtttgttacctgtttattgacatgcctattaacagcgaccatggctcagacgtgttaaaatattcattccatgtcataaacagaactctcc

SEQ ID NO: 495 - RNA, 495 nt, synthetic construct
  source 1..495: mol_type = other RNA
gggtgatatacgaccagttcactcttaaacgaccatggctcagacctgttaaaaccttacattactggtctatatcaataatgtttgataagaaatgacttgatatctacccgaccatggctcagacctgttaaaatgctcctatagtcattcttatcatttactctcattagtatgttaagagactagaatacgaccatggctcagacctgttaaaatatccacaacttaacaactaatgtatgaacctcaatacagattatgaactcactccacgaccatggctcagacctgttaaaacgcgctacacataatcgtattgacttaatgactatatacttcaaggccacctaactgcgaccatggctcagacctgttaaaacttcaccaaccttgaatatatagactgatctctgtttattgacatgacaagaaacttcgaccatggctcagacctgttactaatatacacccatgtcataaacagttccttgt

SEQ ID NO: 496 - RNA, 495 nt, synthetic construct
  source 1..495: mol_type = other RNA
gggtgatatacgaccagttgcacgcactccgaccatggctcagacttgttaaaattatacactactggtctatatcacccttacttgataagaaatgactctaaatgcactcgaccatggctcagacttgttaaatattctacacagtcattcttatcattcgtctccattagtatgttaagatctctatcttcgaccatggctcagacttgttacacttaaattcgcttaacaactaatgtctcaacctcaatacagattatgtgcattaaacccgaccatggctcagacttgttaaaacttccatctcataatcgtattgaacaactatctatatacttcaaggtactagaaatccgaccatggctcagacttgttaaataacctcaatccttgaatatatagtctaaatactgtttattgacatgctctaagaactcgaccatggctcagacttgttaaataacgcatcccatgtcataaacaggctacaac

SEQ ID NO: 497 - RNA, 489 nt, synthetic construct
  source 1..489: mol_type = other RNA
gggtgatatacgaccagtcaaccaactatcgaccatggctcagactgtaacatttaactcaaactggtctatatcaccactccctgataagaaatgactatatactaattcgaccatggctcagactgtaatataagacatcaagtcattcttatcaaaactatccattagtatgttaagtgacacgcacccgaccatggctcagactgtaaattccaaatcctcttaacaactaatgcattccaatcaatacagattatgtaacgtatcaccgaccatggctcagactgtaaacattcctcattcataatcgtattgaacatctacctatatacttcaaggttccctaatgacgaccatggctcagactgtaacaacgacctactccttgaatatatagatactatcctgtttattgacatgtcaatttcaaccgaccatggctcagactgtaatattcacccgctcatgtcataaacagacaacctt

SEQ ID NO: 498 - RNA, 489 nt, synthetic construct
  source 1..489: mol_type = other RNA
gggtgatatacgaccagtaatacacttcacgaccatggctcagactgatattactataaaccactggtctatatcactacaatatgataagaaatgacttttccacactacgaccatggctcagactgatataaatctaacatagtcattcttatcaagcaaccacattagtatgttaagatacataattacgaccatggctcagactgataaccttccttccccttaacaactaatgctttaatatcaatacagattatgtaaacttaactcgaccatggctcagactgatatttaatactaaccataatcgtattgaccctccaactatatacttcaaggctccacacgatcgaccatggctcagactgatataacaattcttaccttgaatatatagcttcatacctgtttattgacatgaatcacaagaacgaccatggctcagactgataaacctaatcctccatgtcataaacagtaccttat

SEQ ID NO: 499 - RNA, 489 nt, synthetic construct
  source 1..489: mol_type = other RNA
gggtgatatacgaccagttcagacaaacacgaccatggctcagactcttactatctcaacacactggtctatatcacatttacatgataagaaatgacttgaacaaacgacgaccatggctcagactcttactaaattctcttagtcattcttatcacatctactcattagtatgttaaggaacagcattccgaccatggctcagactcttagatacagtaccacttaacaactaatgcatcaatatcaatacagattatggtaaactataccgaccatggctcagactcttaattacattcaaacataatcgtattgaccatcttgctatatacttcaaggacacaacgttacgaccatggctcagactcttacaaacttatcacccttgaatatatagatttaaccctgtttattgacatggataccaattacgaccatggctcagactcttacttacacaaccgcatgtcataaacagcacaaccg

SEQ ID NO: 500 - RNA, 489 nt, synthetic construct
  source 1..489: mol_type = other RNA
gggtgatatacgaccagttatacagactacgaccatggctcagacagttacaaacgcgaaccactggtctatatcacacaattttgataagaaatgactttacaaatatccgaccatggctcagacagttaaaaccgcaacatagtcattcttatcatcccgcatcattagtatgttaagctacaaatcaccgaccatggctcagacagttagacaccttccctcttaacaactaatgttctacactcaatacagattatgtaaatcctacccgaccatggctcagacagttaacctctttcactcataatcgtattgataatcaatctatatacttcaaggtacaagcacgacgaccatggctcagacagttaaacctccacaatccttgaatatatagctttaatactgtttattgacatgaagaatccctacgaccatggctcagacagttacataacctacagcatgtcataaacagaacacaga

SEQ ID NO: 501 - RNA, 489 nt, synthetic construct
  source 1..489: mol_type = other RNA
gggtgatatacgaccagttttctatccttcgaccatggctcagagtgttaatactatatcatactggtctatatcatctatctttgataagaaatgactatcctacccgacgaccatggctcagagtgttatatccattgacgagtcattcttatcattaccaaccattagtatgttaagaaatcttccaccgaccatggctcagagtgttaaaccatctacagcttaacaactaatgtgctcatctcaatacagattatgtgttactttcacgaccatggctcagagtgttatctatcatatcccataatcgtattgaagtcctctctatatacttcaaggagcaacctttacgaccatggctcagagtgttaatcttacgaatcccttgaatatatagcgtcaaccctgtttattgacatgtataaatcacgcgaccatggctcagagtgttaccaacagaatttcatgtcataaacagatccttct

SEQ ID NO: 502 - RNA, 489 nt, synthetic construct
  source 1..489: mol_type = other RNA
gggtgatatacgaccagtctgcgaatacccgaccatggctcagtctgttacctttgatcttaactggtctatatcatctacctctgataagaaatgactgtgaacattcccgaccatggctcagtctgttatctaacgaaataagtcattcttatcaaactcaaccattagtatgttaagagatacttactcgaccatggctcagtctgttatcctaccttccgcttaacaactaatgtactcatctcaatacagattatgctccactattccgaccatggctcagtctgttaaaactcgatcttcataatcgtattgatctagccactatatacttcaaggaccaaagcgcccgaccatggctcagtctgttagttaattccataccttgaatatatagccacttagctgtttattgacatggcctctcattgcgaccatggctcagtctgttaattccttccaagcatgtcataaacagaatctcca

SEQ ID NO: 503 - RNA, 489 nt, synthetic construct
  source 1..489: mol_type = other RNA
gggtgatatacgaccagtaccctcatctacgaccatggctcacactgttattcttatcattaactggtctatatcattccaaattgataagaaatgactctgactattcgcgaccatggctcacactgttaaatatgaaacctagtcattcttatcacactcaaccattagtatgttaaggcaatctacttcgaccatggctcacactgttatacaacctacgacttaacaactaatgaagctcagtcaatacagattatgcacgcttcattcgaccatggctcacactgttaattaactaatctcataatcgtattgaattccacactatatacttcaaggactaacacatacgaccatggctcacactgttacaagtcctctccccttgaatatatagccgcctacctgtttattgacatgatttacatctacgaccatggctcacactgttacactcaaattaccatgtcataaacagtacccttc

SEQ ID NO: 504 - RNA, 201 nt, synthetic construct
  source 1..201: mol_type = other RNA
gggtgatatacgaccagttccccgaccatggctcagactgttagaacactggtctatatcatgcgactatgataagaaatgactacgacgaccatggctcagactgttactaaagtcattcttatcaatccgaatcattagtatgttaagcatccgaccatggctcagactgttaattacttaacaactaatgctaccata

SEQ ID NO: 505 - RNA, 213 nt, synthetic construct
  source 1..213: mol_type = other RNA
gggtgatatacgaccagtataacgcgaccatggctcagactgttactacccactggtctatatcatgcccgattgataagaaatgactgacacccgaccatggctcagactgttagcaataagtcattcttatcatgaactcacattagtatgttaagttattccgaccatggctcagactgttaatcactcttaacaactaatgaactaaat

SEQ ID NO: 506 - RNA, 225 nt, synthetic construct
  source 1..225: mol_type = other RNA
gggtgatatacgaccagtaaatgcaacgaccatggctcagactgttaactaaatgactggtctatatcacaccttcctgataagaaatgactccgcacgacgaccatggctcagactgttacatacccaagtcattcttatcatacattctcattagtatgttaagtatcacaacgaccatggctcagactgttaacctacctcttaacaactaatgttacaacc

SEQ ID NO: 507 - RNA, 249 nt, synthetic construct
  source 1..249: mol_type = other RNA
gggtgatatacgaccagtctccctccgaatcgaccatggctcagactgttaactatattacacactggtctatatcaaactcccatgataagaaatgacttgcaacctctcacgaccatggctcagactgttaactccctcaactagtcattcttatcagaccctaccattagtatgttaagcgacaataaacacgaccatggctcagactgttaatctcacaacctcttaacaactaatgcttctatc

SEQ ID NO: 508 - RNA, 399 nt, synthetic construct
  source 1..399: mol_type = other RNA
gggtgatatacgaccagttccccgaccatggctcagactgttagaacactggtctatatcatgcgactatgataagaaatgactacgacgaccatggctcagactgttactaaagtcattcttatcaatccgaatcattagtatgttaagcatccgaccatggctcagactgttaattacttaacaactaatgctaccatatcaatacagattatgttcacgaccatggctcagactgttaataccataatcgtattgactcccatactatatacttcaaggtccgcgaccatggctcagactgttaccttccttgaatatatagagaaattactgtttattgacatgcacacgaccatggctcagactgttaaacccatgtcataaacagactccctg

SEQ ID NO: 509 - RNA, 423 nt, synthetic construct
  source 1..423: mol_type = other RNA
gggtgatatacgaccagtataacgcgaccatggctcagactgttactacccactggtctatatcatgcccgattgataagaaatgactgacacccgaccatggctcagactgttagcaataagtcattcttatcatgaactcacattagtatgttaagttattccgaccatggctcagactgttaatcactcttaacaactaatgaactaaattcaatacagattatgaacttacgaccatggctcagactgttactttcccataatcgtattgagatccatactatatacttcaagggttcgacgaccatggctcagactgttacacataccttgaatatatagaccacaccctgtttattgacatgaataaccgaccatggctcagactgttaaccaagcatgtcataaacagaaccactg

SEQ ID NO: 510 - RNA, 447 nt, synthetic construct
  source 1..447: mol_type = other RNA
gggtgatatacgaccagtaaatgcaacgaccatggctcagactgttaactaaatgactggtctatatcacaccttcctgataagaaatgactccgcacgacgaccatggctcagactgttacatacccaagtcattcttatcatacattctcattagtatgttaagtatcacaacgaccatggctcagactgttaacctacctcttaacaactaatgttacaacctcaatacagattatgacgaaatacgaccatggctcagactgttacaccttaacataatcgtattgaaatccctcctatatacttcaaggtacactcccgaccatggctcagactgttacagacactccttgaatatatagcgcacaacctgtttattgacatgtttaagaacgaccatggctcagactgttacaactcaccatgtcataaacagcccatagc

SEQ ID NO: 511 - RNA, 495 nt, synthetic construct
  source 1..495: mol_type = other RNA
gggtgatatacgaccagtccgatcactctacgaccatggctcagactgttacacatatcctaaactggtctatatcacagctagctgataagaaatgactatctacaacttacgaccatggctcagactgttaccaaacacctaaagtcattcttatcatatctcttcattagtatgttaaggcatcaccgaatcgaccatggctcagactgttaaactcctacaaacttaacaactaatgctcactattcaatacagattatgactatcctaaatcgaccatggctcagactgttaacaatttcaccgcataatcgtattgataccatacctatatacttcaaggcactccttctctcgaccatggctcagactgttacattaatacccaccttgaatatatagcagatcccctgtttattgacatgattactaatctacgaccatggctcagactgttacttaatttcctacatgtcataaacagactctcca

SEQ ID NO: 512 - RNA, 86 nt, synthetic construct
  source 1..86: mol_type = other RNA
gggtgatatacgaccagtgcgcactacacaacccctatcacgtaagcattaagaaactcaagatactggtctatatcattatgagc

SEQ ID NO: 513 - RNA, 169 nt, synthetic construct
  source 1..169: mol_type = other RNA
gggtgatatacgaccagtgcgcactacacaacccctatcacgtaagcattaagaaactcaagatactggtctatatcattatgagctgataagaaatgactatccgaactacaacccctatcacgtaagcattaatcatccaacatgagtcattcttatcattcagaca

SEQ ID NO: 514 - RNA, 252 nt, synthetic construct
  source 1..252: mol_type = other RNA
gggtgatatacgaccagtgcgcactacacaacccctatcacgtaagcattaagaaactcaagatactggtctatatcattatgagctgataagaaatgactatccgaactacaacccctatcacgtaagcattaatcatccaacatgagtcattcttatcattcagacacattagtatgttaagtgactcatcataacccctatcacgtaagcattaactactcaacgatcttaacaactaatgtttagtag

SEQ ID NO: 515 - RNA, 335 nt, synthetic construct
  source 1..335: mol_type = other RNA
gggtgatatacgaccagtgcgcactacacaacccctatcacgtaagcattaagaaactcaagatactggtctatatcattatgagctgataagaaatgactatccgaactacaacccctatcacgtaagcattaatcatccaacatgagtcattcttatcattcagacacattagtatgttaagtgactcatcataacccctatcacgtaagcattaactactcaacgatcttaacaactaatgtttagtagtcaatacagattatgacagcatctataacccctatcacgtaagcattaatacaccacgacgcataatcgtattgattctttca

SEQ ID NO: 516 - RNA, 418 nt, synthetic construct
  source 1..418: mol_type = other RNA
gggtgatatacgaccagtgcgcactacacaacccctatcacgtaagcattaagaaactcaagatactggtctatatcattatgagctgataagaaatgactatccgaactacaacccctatcacgtaagcattaatcatccaacatgagtcattcttatcattcagacacattagtatgttaagtgactcatcataacccctatcacgtaagcattaactactcaacgatcttaacaactaatgtttagtagtcaatacagattatgacagcatctataacccctatcacgtaagcattaatacaccacgacgcataatcgtattgattctttcactatatacttcaaggaccatcactacaacccctatcacgtaagcattaacactactagcagccttgaatatatagttcatgcc

SEQ ID NO: 517 - RNA, 501 nt, synthetic construct; identical to SEQ ID NO: 306
  source 1..501: mol_type = other RNA

SEQ ID NO: 518 - RNA, 234 nt, synthetic construct
  source 1..234: mol_type = other RNA
gggtgatatacgaccagtaacatataaccaacccctatcacgtaagcattaaactaactattccactggtctatatcacctgataagaaatgacttacattctcaaaacccctatcacgtaagcattaagatacaccgaacagtcattcttatcacacattagtatgttaagatatagcaacaaacccctatcacgtaagcattaacctcacagaccacttaacaactaatgcc

SEQ ID NO: 519 - RNA, 240 nt, synthetic construct
  source 1..240: mol_type = other RNA
gggtgatatacgaccagttaaagcaatctaacccctatcacgtaagcattaagcacctctcactactggtctatatcacctatgataagaaatgactttccactcgcaaacccctatcacgtaagcattaatacctacctcatagtcattcttatcaataacattagtatgttaagtaatccactctaacccctatcacgtaagcattaaaccactctgacccttaacaactaatgccaa

SEQ ID NO: 520 - RNA, 246 nt, synthetic construct
  source 1..246: mol_type = other RNA
gggtgatatacgaccagtcaacctaattcaacccctatcacgtaagcattaaccatctcctaaaactggtctatatcatatacatgataagaaatgactcattcttagccaacccctatcacgtaagcattaatcgatccgccaaagtcattcttatcacaaccgcattagtatgttaagactctactctaaacccctatcacgtaagcattaattatactccacccttaacaactaatgatctat

SEQ ID NO: 521 - RNA, 252 nt, synthetic construct
  source 1..252: mol_type = other RNA
gggtgatatacgaccagtcccgaccagttaacccctatcacgtaagcattaaatgaacagaaatactggtctatatcatcaaatcctgataagaaatgactttaccacattcaacccctatcacgtaagcattaattaccttccatcagtcattcttatcaccatcacccattagtatgttaagtacattccaagaacccctatcacgtaagcattaaatcacgactcgccttaacaactaatgcaattcaa

SEQ ID NO: 522 - RNA, 465 nt, synthetic construct
  source 1..465: mol_type = other RNA
gggtgatatacgaccagtaacatataaccaacccctatcacgtaagcattaaactaactattccactggtctatatcacctgataagaaatgacttacattctcaaaacccctatcacgtaagcattaagatacaccgaacagtcattcttatcacacattagtatgttaagatatagcaacaaacccctatcacgtaagcattaacctcacagaccacttaacaactaatgcctcaatacagattatgaaactatatccaacccctatcacgtaagcattaatgtcgccacacccataatcgtattgaacctatatacttcaaggatacaatcaaaaacccctatcacgtaagcattaaacaaatacattgccttgaatatatagctctgtttattgacatgcccattatatcaacccctatcacgtaagcattaaagtctctgacctcatgtcataaacagcg

SEQ ID NO: 523 - RNA, 477 nt, synthetic construct
  source 1..477: mol_type = other RNA
gggtgatatacgaccagttaaagcaatctaacccctatcacgtaagcattaagcacctctcactactggtctatatcacctatgataagaaatgactttccactcgcaaacccctatcacgtaagcattaatacctacctcatagtcattcttatcaataacattagtatgttaagtaatccactctaacccctatcacgtaagcattaaaccactctgacccttaacaactaatgccaatcaatacagattatgcacttcaatttaacccctatcacgtaagcattaaccaacctttcttcataatcgtattgaccatctatatacttcaaggctaccctaatcaacccctatcacgtaagcattaaccttcaccctcaccttgaatatatagcgccctgtttattgacatgtccgtaaaccgaacccctatcacgtaagcattaagacccaatcaatcatgtcataaacagtcta

SEQ ID NO: 524 - RNA, 489 nt, synthetic construct
  source 1..489: mol_type = other RNA
gggtgatatacgaccagtcaacctaattcaacccctatcacgtaagcattaaccatctcctaaaactggtctatatcatatacatgataagaaatgactcattcttagccaacccctatcacgtaagcattaatcgatccgccaaagtcattcttatcacaaccgcattagtatgttaagactctactctaaacccctatcacgtaagcattaattatactccacccttaacaactaatgatctattcaatacagattatgaacctcaattaaacccctatcacgtaagcattaacctttcaatatccataatcgtattgatttcaactatatacttcaaggcccacaccgtcaacccctatcacgtaagcattaaccgttccatcacccttgaatatatagaccaaactgtttattgacatgaaaccttaataaacccctatcacgtaagcattaacatattccctagcatgtcataaacagctctat

SEQ ID NO: 525 - RNA, 501 nt, synthetic construct
  source 1..501: mol_type = other RNA
gggtgatatacgaccagtacattccgcaaaacccctatcacgtaagcattaacaactgaccgaaactggtctatatcatataaccttgataagaaatgacttcacttcagccaacccctatcacgtaagcattaagatataaccaatagtcattcttatcacaattctccattagtatgttaagccaccgtcgcaaacccctatcacgtaagcattaaacctagtttcatcttaacaactaatgactttctctcaatacagattatgtaactttaaccaacccctatcacgtaagcattaattagccacaaatcataatcgtattgatattccttctatatacttcaaggtaattaccgtcaacccctatcacgtaagcattaaccataatcgactccttgaatatatagtatagactctgtttattgacatgcctccaattctaacccctatcacgtaagcattaaatacaacttcaacatgtcataaacagcccagcac

SEQ ID NO: 526 - RNA, 237 nt, synthetic construct
  source 1..237: mol_type = other RNA
gggtatacgaccagttttcaatacttaacccctatcacgtaagcattaactaccgcaactcactggtcgtatatttacctctgataagaaatgaccgtaccctcaacccctatcacgtaagcattaaaccgactaataccatttcttatcacccttatacattagtatgttcacgccaataaaacccctatcacgtaagcattaaagtcctaccaataacatactaatgatttattc

SEQ ID NO: 527 - RNA, 234 nt, synthetic construct
  source 1..234: mol_type = other RNA
gggtatacgaccagtgccctatacttaacccctatcacgtaagcattaacgacctctaagaactggtgtatatccctctatgataagaaatgacagtagcattaacccctatcacgtaagcattaaattccctctagccatttctatcaattatcagcattagtatgtttcttccgctccaacccctatcacgtaagcattaatttatccgccataacatataatgacaatcca

SEQ ID NO: 528 - RNA, 237 nt, synthetic construct
  source 1..237: mol_type = other RNA
gggtatacgaccagtccatatccatcaacccctatcacgtaagcattaatatcctaagatcactggtggtatacgttccactgataagaaatgcctcaagaacaaacccctatcacgtaagcattaatgacttccaatacatttcatatcaaccatcaccattagtatgtttattccatctcaacccctatcacgtaagcattaatccatttcgactaacatagtaatgtcaccata

SEQ ID NO: 529 - RNA, 255 nt, synthetic construct
  source 1..255: mol_type = other RNA
gggtgatatacgaccagtctatttcctagaacccctatcacgtaagcattaaagactcacaaccactggtcgtatatcaccgacttctgataagaaatgactcaaatactaataacccctatcacgtaagcattaaattcaagaactcagtcatttcttatcaaatattcccattagtatgttaagatcatttaccgaacccctatcacgtaagcattaaacagaccacaaacttaacatactaatgtgttacgt

SEQ ID NO: 530 - RNA, 252 nt, synthetic construct
  source 1..252: mol_type = other RNA
gggtgatatacgaccagtttaacaaataaaacccctatcacgtaagcattaagtaactatctctactggtctatatcaaagaaatctgataagaaatgactacccttcagcaaacccctatcacgtaagcattaaattccctcttcaagtcattcttatcacaacatgtcattagtatgttaagtcaatctagcaaacccctatcacgtaagcattaaacaacttgtcctcttaacaactaatgtcctcaca

SEQ ID NO: 531 - RNA, 255 nt, synthetic construct
  source 1..255: mol_type = other RNA
gggtgatatacgaccagtttaattccaacaacccctatcacgtaagcattaaagtaaatcgcacactggtcctatatcaacccaacctgataagaaatgacttagcctaacataacccctatcacgtaagcattaagtccgtaaccacagtcattacttatcatgagtcctcattagtatgttaagtctcagccagaaacccctatcacgtaagcattaaatcaatctatatcttaacaaactaatgtcaccgtt

SEQ ID NO: 532 - RNA, 471 nt, synthetic construct
  source 1..471: mol_type = other RNA
gggtatacgaccagtttaaccatccaaacccctatcacgtaagcattaagagccactatttactggtcgtataccattgcatgataagaaatgcccgatctgtcaacccctatcacgtaagcattaagtaccaaccctacatttcttatcacatacacccattagtatgttcctccacgcctaacccctatcacgtaagcattaaccttcgaaatctaacatactaatgttcatacctcaatacagattagacccaacctaacccctatcacgtaagcattaaacaacttcacccaatctgtattgatcccacacctatatacttcagaatacaaccgaacccctatcacgtaagcattaacctctagcccagtgaagtatatagatcacaagctgtttattgacccatattcaagaacccctatcacgtaagcattaactcaacgatttagtcaataaacagacaccaga

SEQ ID NO: 533 - RNA, 465 nt, synthetic construct
  source 1..465: mol_type = other RNA
gggtatacgaccagtgccctatacttaacccctatcacgtaagcattaacgacctctaagaactggtgtatatccctctatgataagaaatgacagtagcattaacccctatcacgtaagcattaaattccctctagccatttctatcaattatcagcattagtatgtttcttccgctccaacccctatcacgtaagcattaatttatccgccataacatataatgacaatccatcaatacagattcaattcaaactaacccctatcacgtaagcattaaccacttcccgtaaatctgattgaaccttcagctatatacttcataatattccgcaacccctatcacgtaagcattaaaagaacaaactttgaagttatagatacctcactgtttattgaccccaagcacttaacccctatcacgtaagcattaaaacgaccagtccgtcaataacagacaattta

SEQ ID NO: 534 - RNA, 471 nt, synthetic construct
  source 1..471: mol_type = other RNA
gggtatacgaccagtccatatccatcaacccctatcacgtaagcattaatatcctaagatcactggtggtatacgttccactgataagaaatgcctcaagaacaaacccctatcacgtaagcattaatgacttccaatacatttcatatcaaccatcaccattagtatgtttattccatctcaacccctatcacgtaagcattaatccatttcgactaacatagtaatgtcaccatatcaatacagattcatataaataaaacccctatcacgtaagcattaaattcccaactctaatctgaattgaaattacttctatatacttcattcagcccagcaacccctatcacgtaagcattaacccacagtcacctgaagtttatagtccagaaactgtttattgacatcagtcccagaacccctatcacgtaagcattaaatccaatagtaagtcaattaacaggccaatat

SEQ ID NO: 535 - RNA, 507 nt, synthetic construct
  source 1..507: mol_type = other RNA
gggtgatatacgaccagtctatttcctagaacccctatcacgtaagcattaaagactcacaaccactggtcgtatatcaccgacttctgataagaaatgactcaaatactaataacccctatcacgtaagcattaaattcaagaactcagtcatttcttatcaaatattcccattagtatgttaagatcatttaccgaacccctatcacgtaagcattaaacagaccacaaacttaacatactaatgtgttacgttcaatacagattatgttccacgcagaaacccctatcacgtaagcattaacaaagaccgactcataatctgtattgaccaatactctatatacttcaaggaacaaacaccgaacccctatcacgtaagcattaatttcacatagaaccttgaagtatatagtgctcactctgtttattgacatgccgtaccgcccaacccctatcacgtaagcattaaataacagtcaaccatgtcaataaacagcaaattac

SEQ ID NO: 536 - RNA, 501 nt, synthetic construct
  source 1..501: mol_type = other RNA
gggtgatatacgaccagtttaacaaataaaacccctatcacgtaagcattaagtaactatctctactggtctatatcaaagaaatctgataagaaatgactacccttcagcaaacccctatcacgtaagcattaaattccctcttcaagtcattcttatcacaacatgtcattagtatgttaagtcaatctagcaaacccctatcacgtaagcattaaacaacttgtcctcttaacaactaatgtcctcacatcaatacagattatgtcgcctgtcctaacccctatcacgtaagcattaaatccctctaattcataatcgtattgactaactcgctatatacttcaaggaccgactcacaaacccctatcacgtaagcattaagaaatcactgaaccttgaatatatagatataaacctgtttattgacatgccacttcattaaacccctatcacgtaagcattaaccattcgtctcacatgtcataaacagtatccagt

SEQ ID NO: 537 - RNA, 507 nt, synthetic construct
  source 1..507: mol_type = other RNA
gggtgatatacgaccagtttaattccaacaacccctatcacgtaagcattaaagtaaatcgcacactggtcctatatcaacccaacctgataagaaatgacttagcctaacataacccctatcacgtaagcattaagtccgtaaccacagtcattacttatcatgagtcctcattagtatgttaagtctcagccagaaacccctatcacgtaagcattaaatcaatctatatcttaacaaactaatgtcaccgtttcaatacagattatgaacatctcaagaacccctatcacgtaagcattaaaccaaacatcgacataatcagtattgatttcattactatatacttcaaggctccataccaaaacccctatcacgtaagcattaaaactcgaagacaccttgaactatatagaccgcgtactgtttattgacatgaaaccgaactaaacccctatcacgtaagcattaatatcatgaccgacatgtcattaaacagccttcctg

SEQ ID NO: 538 - RNA, 255 nt, synthetic construct
  source 1..255: mol_type = other RNA
gggtgatatacgaccagtgcgcactacacaacccctatcacgattagcattaagaaactcaagatactggtctatatcattatgagctgataagaaatgactactttaactacaacccctatcacgattagcattaagaacacgacatcagtcattcttatcattcagacacattagtatgttaagttaccgatcataacccctatcacgattagcattaacttacaaacgatcttaacaactaatgtttagtag

SEQ ID NO: 539 - RNA, 258 nt, synthetic construct
  source 1..258: mol_type = other RNA
gggtgatatacgaccagtgcgcactacacaacccctatcacgattagcaattaagaaactcaagatactggtctatatcattatgagctgataagaaatgactactttaactacaacccctatcacgattagcaattaagaacacgacatcagtcattcttatcattcagacacattagtatgttaagttaagcatcataacccctatcacgattagcaattaacaacgaaacgatcttaacaactaatgtttagtag

SEQ ID NO: 540 - RNA, 258 nt, synthetic construct
  source 1..258: mol_type = other RNA
gggtgatatacgaccagttcatactacacaacccctatcacgattagcgattaagaaactcaatatactggtctatatcattatgagctgataagaaatgactacaccgacataaacccctatcacgattagcgattaacctacagagattagtcattcttatcattcagacacattagtatgttaagtgactcatcataacccctatcacgattagcgattaaattcagaacgatcttaacaactaatgtttagtag

SEQ ID NO: 541 - RNA, 258 nt, synthetic construct
  source 1..258: mol_type = other RNA
gggtgatatacgaccagtgcgcactacacaacccctatcacgattagccattaagaaactcaagatactggtctatatcattatgagctgataagaaatgactactttaactacaacccctatcacgattagccattaagaacacgacatcagtcattcttatcattcagacacattagtatgttaagttaagcatcataacccctatcacgattagccattaacaacgaaacgatcttaacaactaatgtttagtag

SEQ ID NO: 542 - RNA, 258 nt, synthetic construct
  source 1..258: mol_type = other RNA
gggtgatatacgaccagtgcgcactacacaacccctatcacgattagctattaagaaactcaagatactggtctatatcattatgagctgataagaaatgactactttaactacaacccctatcacgattagctattaagaacacgacatcagtcattcttatcattcagacacattagtatgttaagttaccgatcataacccctatcacgattagctattaacttacaaacgatcttaacaactaatgtttagtag

SEQ ID NO: 543 - RNA, 255 nt, synthetic construct
  source 1..255: mol_type = other RNA
gggtgatatacgaccagtattcttaacccaacccctatcacgattagcatttacacttcgcatcaactggtctatatcattatgagctgataagaaatgactaccatcacctcaacccctatcacgattagcatttataactatatttaagtcattcttatcattcagacacattagtatgttaagttctcacgaccaacccctatcacgattagcatttaaatagcacatatcttaacaactaatgtttagtag

SEQ ID NO: 544 - RNA, 255 nt, synthetic construct
  source 1..255: mol_type = other RNA
gggtgatatacgaccagtgtcacataaataacccctatcacgattagcataaaaccgagaataaaactggtctatatcattatgagctgataagaaatgactccatcacttcaaacccctatcacgattagcataaaaattctcaatctagtcattcttatcattcagacacattagtatgttaagcattcaaccctaacccctatcacgattagcataaattacgcctttcccttaacaactaatgtttagtag

SEQ ID NO: 545 - RNA, 255 nt, synthetic construct
  source 1..255: mol_type = other RNA
gggtgatatacgaccagttttcagcctcaaacccctatcacgattagcaataactccttcccaccactggtctatatcattatgagctgataagaaatgactccaacctctaaaacccctatcacgattagcaataacactcaaactctagtcattcttatcattcagacacattagtatgttaagagaaacgccttaacccctatcacgattagcaataaatactgagccaacttaacaactaatgtttagtag

SEQ ID NO: 546 - RNA, 255 nt, synthetic construct
  source 1..255: mol_type = other RNA
gggtgatatacgaccagtctaacttactaaacccctatcacgattagctttaaatatcacattccactggtctatatcattatgagctgataagaaatgactcgaatttccgcaacccctatcacgattagctttaacctataccacacagtcattcttatcattcagacacattagtatgttaagtaaacactaacaacccctatcacgattagctttaactcactttcgaccttaacaactaatgtttagtag

SEQ ID NO: 547 - RNA, 255 nt, synthetic construct
  source 1..255: mol_type = other RNA
gggtgatatacgaccagtttaacatttcaaacccctatcacgattaggattaacatcacaacctcactggtctatatcattatgagctgataagaaatgacttaaactcactcaacccctatcacgattaggattaataccgctaccctagtcattcttatcattcagacacattagtatgttaagcccatacatcaaacccctatcacgattaggattaaatacacgccaaccttaacaactaatgtttagtag

SEQ ID NO: 548 - RNA, 255 nt, synthetic construct
  source 1..255: mol_type = other RNA
gggtgatatacgaccagtattctacaaccaacccctatcacgattaccattaactatagcctcacactggtctatatcattatgagctgataagaaatgactcataaacacataacccctatcacgattaccattaaccagcataacacagtcattcttatcattcagacacattagtatgttaagcgctctactttaacccctatcacgattaccattaactaacgaaacaccttaacaactaatgtttagtag

SEQ ID NO: 549 - RNA, 255 nt, synthetic construct
  source 1..255: mol_type = other RNA
gggtgatatacgaccagtagactccacgaaacccctatcacgatttgcattaacatacacttaacactggtctatatcattatgagctgataagaaatgactgaactacatataacccctatcacgatttgcattaaatacacccactaagtcattcttatcattcagacacattagtatgttaagtaaccttacgaaacccctatcacgatttgcattaataaacaatataccttaacaactaatgtttagtag

SEQ ID NO: 550 - RNA, 255 nt, synthetic construct
  source 1..255: mol_type = other RNA
gggtgatatacgaccagtgcgcactacacaacccctatcacgtaagcaattaagaaactcaagatactggtctatatcattatgagctgataagaaatgactatccgaactacaacccctatcacgtaagcaattaatcatccaacatgagtcattcttatcattcagacacattagtatgttaagtgactcatcataacccctatcacgtaagcaattaactactcaacgatcttaacaactaatgtttagtag

SEQ ID NO: 551 - RNA, 255 nt, synthetic construct
  source 1..255: mol_type = other RNA
gggtgatatacgaccagttcagactacacaacccctatcacgtaagcgattaagaaactcaatatactggtctatatcattatgagctgataagaaatgactacaccgacataaacccctatcacgtaagcgattaacctacagagattagtcattcttatcattcagacacattagtatgttaagtgactcagcataacccctatcacgtaagcgattaaattcagaacaatcttaacaactaatgtttagtag

SEQ ID NO: 552 - RNA, 255 nt, synthetic construct
  source 1..255: mol_type = other RNA
gggtgatatacgaccagtgcgcactacacaacccctatcacgtaagccattaagaaactcaagatactggtctatatcattatgagctgataagaaatgactatccgaactacaacccctatcacgtaagccattaatcatccaacatgagtcattcttatcattcagacacattagtatgttaagtgactcatcataacccctatcacgtaagccattaactactcaacgatcttaacaactaatgtttagtag

SEQ ID NO: 553 - RNA, 255 nt, synthetic construct
  source 1..255: mol_type = other RNA
gggtgatatacgaccagtgcgcactacacaacccctatcacgtaagctattaagaaactcaagatactggtctatatcattatgagctgataagaaatgactatccgaactacaacccctatcacgtaagctattaatcatccaacatgagtcattcttatcattcagacacattagtatgttaagtgactcatcataacccctatcacgtaagctattaactactcaacgatcttaacaactaatgtttagtag

SEQ ID NO: 554 - RNA, 252 nt, synthetic construct
  source 1..252: mol_type = other RNA
gggtgatatacgaccagtaccatctccgcaacccctatcacgtaagcatttaaagttcccagccactggtctatatcaaacaccaatgataagaaatgactttacattaacgaacccctatcacgtaagcatttaatataattacttagtcattcttatcagaaactaccattagtatgttaagcacttaaaccaaacccctatcacgtaagcatttaccaaatcgactacttaacaactaatgccaactta

SEQ ID NO: 555 - RNA, 252 nt, synthetic construct
  source 1..252: mol_type = other RNA
gggtgatatacgaccagtcctcaatcgcaaacccctatcacgtaagcataaacctaaacatactactggtctatatcacaattaactgataagaaatgactcgccataacagaacccctatcacgtaagcataaagaaacttccattagtcattcttatcaacctgaaccattagtatgttaagcaactccaataaacccctatcacgtaagcataaacaagaccgaccacttaacaactaatgagccgcat

SEQ ID NO: 556 - RNA, 252 nt, synthetic construct
  source 1..252: mol_type = other RNA
gggtgatatacgaccagtaaaccaacctaaacccctatcacgtaagcaataagccatttccaccactggtctatatcagaaaccactgataagaaatgactatccatcagtcaacccctatcacgtaagcaataacaccattaagccagtcattcttatcaatcttcaccattagtatgttaagattccgcagtcaacccctatcacgtaagcaataacatcttaacttccttaacaactaatgctacttcc

SEQ ID NO: 557 - RNA, 252 nt, synthetic construct
  source 1..252: mol_type = other RNA
gggtgatatacgaccagtcgacatctactaacccctatcacgtaagctttaataatctacctaaactggtctatatcaatcattcctgataagaaatgactcctcccattaaaacccctatcacgtaagctttaaattagacataacagtcattcttatcaatacctaccattagtatgttaagcatcctatactaacccctatcacgtaagctttaaccttctgtacctcttaacaactaatgaactaccc

SEQ ID NO: 558 - RNA, 252 nt, synthetic construct
  source 1..252: mol_type = other RNA
gggtgatatacgaccagtctcacttaaacaacccctatcacgtaaggattaaagacaccgcacaactggtctatatcatgctaaactgataagaaatgactgcctttaacacaacccctatcacgtaaggattaataacgcccaatgagtcattcttatcacctttgttcattagtatgttaagacccaagatcaaacccctatcacgtaaggattaaataacgacaaaccttaacaactaatgccacctgt

SEQ ID NO: 559 - RNA, 252 nt, synthetic construct
  source 1..252: mol_type = other RNA
gggtgatatacgaccagtccaacacccgaaacccctatcacgtaaccattaagcattccgattcactggtctatatcaatctcacctgataagaaatgactaaatccattataacccctatcacgtaaccattaactcctgtctagaagtcattcttatcaaactaatccattagtatgttaagaaatcactataaacccctatcacgtaaccattaattaatcaatcaccttaacaactaatgattcaacc

SEQ ID NO: 560 - RNA, 252 nt, synthetic construct
  source 1..252: mol_type = other RNA
gggtgatatacgaccagtactccaatcaaaacccctatcacgtatgcattaactacttacttcaactggtctatatcatctctacctgataagaaatgactacacgcaatcaaacccctatcacgtatgcattaaatcacaaccctcagtcattcttatcacacattcccattagtatgttaagaatttctctacaacccctatcacgtatgcattaatcaaactaatcgcttaacaactaatgaatcaatt

SEQ ID NO: 561 - RNA, 507 nt, synthetic construct
  source 1..507: mol_type = other RNA
gggtgatatacgaccagtgcgcactacacaacccctatcacgattagcattaagaaactcaagatactggtctatatcattatgagctgataagaaatgactactttaactacaacccctatcacgattagcattaagaacacgacatcagtcattcttatcattcagacacattagtatgttaagttaccgatcataacccctatcacgattagcattaacttacaaacgatcttaacaactaatgtttagtagtcaatacagattatgacatcacgcataacccctatcacgattagcattaatacaccacgacgcataatcgtattgattctttcactatatacttcaaggactctatctacaacccctatcacgattagcattaacacttcacgcctccttgaatatatagttcatgccctgtttattgacatgtcacatctcacaacccctatcacgattagcattaacacatcacgcatcatgtcataaacagttatacaa

SEQ ID NO: 562 - RNA, 507 nt, synthetic construct
  source 1..507: mol_type = other RNA
gggtgatatacgaccagtattcttaacccaacccctatcacgattagcatttacacttcgcatcaactggtctatatcattatgagctgataagaaatgactaccatcacctcaacccctatcacgattagcatttataactatatttaagtcattcttatcattcagacacattagtatgttaagttctcacgaccaacccctatcacgattagcatttaaatagcacatatcttaacaactaatgtttagtagtcaatacagattatggaaatacatacaacccctatcacgattagcatttaagaaatccatatcataatcgtattgattctttcactatatacttcaaggacctctaacataacccctatcacgattagcatttaactatcctctcaccttgaatatatagttcatgccctgtttattgacatgtcacgcaccgaaacccctatcacgattagcatttattacaaacctaccatgtcataaacagttatacaa

SEQ ID NO: 563 - RNA, 507 nt, synthetic construct
  source 1..507: mol_type = other RNA
gggtgatatacgaccagtgtcacataaataacccctatcacgattagcataaaaccgagaataaaactggtctatatcattatgagctgataagaaatgactccatcacttcaaacccctatcacgattagcataaaaattctcaatctagtcattcttatcattcagacacattagtatgttaagcattcaaccctaacccctatcacgattagcataaattacgcctttcccttaacaactaatgtttagtagtcaatacagattatgattatccctctaacccctatcacgattagcataaactatccagcctccataatcgtattgattctttcactatatacttcaaggaccacaccaataacccctatcacgattagcataaacctaaaccattaccttgaatatatagttcatgccctgtttattgacatgatcttaacctcaacccctatcacgattagcataaacaagactttcaccatgtcataaacagttatacaa

SEQ ID NO: 564 - RNA, 507 nt, synthetic construct
  source 1..507: mol_type = other RNA
gggtgatatacgaccagttttcagcctcaaacccctatcacgattagcaataactccttcccaccactggtctatatcattatgagctgataagaaatgactccaacctctaaaacccctatcacgattagcaataacactcaaactctagtcattcttatcattcagacacattagtatgttaagagaaacgccttaacccctatcacgattagcaataaatactgagccaacttaacaactaatgtttagtagtcaatacagattatgaactaacattaaacccctatcacgattagcaataatagcactcaagacataatcgtattgattctttcactatatacttcaaggcctaccttaacaacccctatcacgattagcaataatacaatcaatacccttgaatatatagttcatgccctgtttattgacatgaccactcaaccaacccctatcacgattagcaataacaaatcctacaccatgtcataaacagttatacaa

SEQ ID NO: 565 - RNA, 507 nt, synthetic construct
  source 1..507: mol_type = other RNA
gggtgatatacgaccagtctaacttactaaacccctatcacgattagctttaaatatcacattccactggtctatatcattatgagctgataagaaatgactcgaatttccgcaacccctatcacgattagctttaacctataccacacagtcattcttatcattcagacacattagtatgttaagtaaacactaacaacccctatcacgattagctttaactcactttcgaccttaacaactaatgtttagtagtcaatacagattatgtcacttacactaacccctatcacgattagctttaatttcatctacgtcataatcgtattgattctttcactatatacttcaagggagatcttactaacccctatcacgattagctttaaatactcaataagccttgaatatatagttcatgccctgtttattgacatgtccaccgcgcaaacccctatcacgattagctttaaatacaatccacccatgtcataaacagttatacaa

SEQ ID NO: 566 - RNA, 507 nt, synthetic construct
  source 1..507: mol_type = other RNA
gggtgatatacgaccagtttaacatttcaaacccctatcacgattaggattaacatcacaacctcactggtctatatcattatgagctgataagaaatgacttaaactcactcaacccctatcacgattaggattaataccgctaccctagtcattcttatcattcagacacattagtatgttaagcccatacatcaaacccctatcacgattaggattaaatacacgccaaccttaacaactaatgtttagtagtcaatacagattatgactctcacaacaacccctatcacgattaggattaaagacccacaacccataatcgtattgattctttcactatatacttcaaggcaaatattaagaacccctatcacgattaggattaaaaagctctcatcccttgaatatatagttcatgccctgtttattgacatgctctttaacacaacccctatcacgattaggattaaaaaccgcacactcatgtcataaacagttatacaa

SEQ ID NO: 567 - RNA, 507 nt, synthetic construct
  source 1..507: mol_type = other RNA
gggtgatatacgaccagtattctacaaccaacccctatcacgattaccattaactatagcctcacactggtctatatcattatgagctgataagaaatgactcataaacacataacccctatcacgattaccattaaccagcataacacagtcattcttatcattcagacacattagtatgttaagcgctctactttaacccctatcacgattaccattaactaacgaaacaccttaacaactaatgtttagtagtcaatacagattatgtcagcctccaaaacccctatcacgattaccattaacccgtatcataccataatcgtattgattctttcactatatacttcaaggagaaatctatcaacccctatcacgattaccattaaccataagcacaaccttgaatatatagttcatgccctgtttattgacatgaccatcacatgaacccctatcacgattaccattaaacacgacccacacatgtcataaacagttatacaa

SEQ ID NO: 568 - RNA, 507 nt, synthetic construct
  source 1..507: mol_type = other RNA
gggtgatatacgaccagtagactccacgaaacccctatcacgatttgcattaacatacacttaacactggtctatatcattatgagctgataagaaatgactgaactacatataacccctatcacgatttgcattaaatacacccactaagtcattcttatcattcagacacattagtatgttaagtaaccttacgaaacccctatcacgatttgcattaataaacaatataccttaacaactaatgtttagtagtcaatacagattatgccataactccgaacccctatcacgatttgcattaatactattcagtccataatcgtattgattctttcactatatacttcaaggacaacctcccaaacccctatcacgatttgcattaaccactccatataccttgaatatatagttcatgccctgtttattgacatgaagaaccaagaaacccctatcacgatttgcattaaccacaatatagccatgtcataaacagttatacaa

SEQ ID NO: 569 - RNA, 501 nt, synthetic construct
  source 1..501: mol_type = other RNA
gggtgatatacgaccagtaccatctccgcaacccctatcacgtaagcatttaaagttcccagccactggtctatatcaaacaccaatgataagaaatgactttacattaacgaacccctatcacgtaagcatttaatataattacttagtcattcttatcagaaactaccattagtatgttaagcacttaaaccaaacccctatcacgtaagcatttaccaaatcgactacttaacaactaatgccaacttatcaatacagattatgccctagaccataacccctatcacgtaagcatttaactataacccaccataatcgtattgaacgttcccctatatacttcaaggtaaattccaagaacccctatcacgtaagcatttagccagatcaactccttgaatatatagcgctcttcctgtttattgacatgtaataccgactaacccctatcacgtaagcatttatttattcaccgccatgtcataaacagcatacctt

SEQ ID NO: 570 - RNA, 501 nt, synthetic construct
  source 1..501: mol_type = other RNA
gggtgatatacgaccagtcctcaatcgcaaacccctatcacgtaagcataaacctaaacatactactggtctatatcacaattaactgataagaaatgactcgccataacagaacccctatcacgtaagcataaagaaacttccattagtcattcttatcaacctgaaccattagtatgttaagcaactccaataaacccctatcacgtaagcataaacaagaccgaccacttaacaactaatgagccgcattcaatacagattatgaacgcaccaataacccctatcacgtaagcataaaaatatcaacacccataatcgtattgatcaatcatctatatacttcaaggacacccacagcaacccctatcacgtaagcataaagaaattcgaccaccttgaatatatagctacgtacctgtttattgacatgcaagaacgcccaacccctatcacgtaagcataaatagaacttaagacatgtcataaacagtctccgag

SEQ ID NO: 571 - RNA, 501 nt, synthetic construct
  source 1..501: mol_type = other RNA
gggtgatatacgaccagtaaaccaacctaaacccctatcacgtaagcaataagccatttccaccactggtctatatcagaaaccactgataagaaatgactatccatcagtcaacccctatcacgtaagcaataacaccattaagccagtcattcttatcaatcttcaccattagtatgttaagattccgcagtcaacccctatcacgtaagcaataacatcttaacttccttaacaactaatgctacttcctcaatacagattatgcaattcgcagaaacccctatcacgtaagcaataaaagacataccaacataatcgtattgattcaaatcctatatacttcaaggctcaatttaagaacccctatcacgtaagcaataaacatattcacttccttgaatatatagagtcatctctgtttattgacatgattaatccgcaaacccctatcacgtaagcaataactcccagaattacatgtcataaacagctacctta

SEQ ID NO: 572 - RNA, 501 nt, synthetic construct
  source 1..501: mol_type = other RNA
gggtgatatacgaccagtcgacatctactaacccctatcacgtaagctttaataatctacctaaactggtctatatcaatcattcctgataagaaatgactcctcccattaaaacccctatcacgtaagctttaaattagacataacagtcattcttatcaatacctaccattagtatgttaagcatcctatactaacccctatcacgtaagctttaaccttctgtacctcttaacaactaatgaactaccctcaatacagattatgccacgactaacaacccctatcacgtaagctttaatttccacatctacataatcgtattgaaacacaacctatatacttcaaggatacaaaccgcaacccctatcacgtaagctttaaatgacccaccgcccttgaatatatagcagaaacactgtttattgacatgcataaattcttaacccctatcacgtaagctttaaactcaccattaacatgtcataaacagaatagcta

SEQ ID NO: 573 - RNA, 501 nt, synthetic construct
  source 1..501: mol_type = other RNA
gggtgatatacgaccagtctcacttaaacaacccctatcacgtaaggattaaagacaccgcacaactggtctatatcatgctaaactgataagaaatgactgcctttaacacaacccctatcacgtaaggattaataacgcccaatgagtcattcttatcacctttgttcattagtatgttaagacccaagatcaaacccctatcacgtaaggattaaataacgacaaaccttaacaactaatgccacctgttcaatacagattatgacccacattcaaacccctatcacgtaaggattaaacacgcctcaaacataatcgtattgattccaatactatatacttcaaggatacagaacacaacccctatcacgtaaggattaatttagcacacgaccttgaatatatagattatcccctgtttattgacatgcacaaactacaaacccctatcacgtaaggattaatacacataacatcatgtcataaacagcctcaaac

SEQ ID NO: 574 - RNA, 501 nt, synthetic construct
  source 1..501: mol_type = other RNA
gggtgatatacgaccagtccaacacccgaaacccctatcacgtaaccattaagcattccgattcactggtctatatcaatctcacctgataagaaatgactaaatccattataacccctatcacgtaaccattaactcctgtctagaagtcattcttatcaaactaatccattagtatgttaagaaatcactataaacccctatcacgtaaccattaattaatcaatcaccttaacaactaatgattcaacctcaatacagattatgcatactctcccaacccctatcacgtaaccattaagatcgcaaataccataatcgtattgaaactgcaactatatacttcaaggacccgtctcctaacccctatcacgtaaccattaaccaaccctgtacccttgaatatatagcaaatagcctgtttattgacatgccacaccaagaaacccctatcacgtaaccattaaactagaacgaaacatgtcataaacagtacataca

SEQ ID NO: 575 - RNA, 501 nt, synthetic construct
  source 1..501: mol_type = other RNA
gggtgatatacgaccagtactccaatcaaaacccctatcacgtatgcattaactacttacttcaactggtctatatcatctctacctgataagaaatgactacacgcaatcaaacccctatcacgtatgcattaaatcacaaccctcagtcattcttatcacacattcccattagtatgttaagaatttctctacaacccctatcacgtatgcattaatcaaactaatcgcttaacaactaatgaatcaatttcaatacagattatgagacttatattaacccctatcacgtatgcattaatcttcgcgccaacataatcgtattgacaatcctactatatacttcaagggcaatccttctaacccctatcacgtatgcattaactcaagctaagaccttgaatatatagccttatttctgtttattgacatgctatcagcttcaacccctatcacgtatgcattaacactttctcattcatgtcataaacagtcaacgtt

SEQ ID NO: 576 - RNA, 207 nt, synthetic construct
  source 1..207: mol_type = other RNA
gggtgatatacgaccagtgacgaacccctatcacgtaagcattaaataaactggtctatatcacaagactatgataagaaatgactgaagaacccctatcacgtaagcattaacctaagtcattcttatcagctatttccattagtatgttaagagaaaacccctatcacgtaagcattaaatgacttaacaactaatgaattacaa

SEQ ID NO: 577 - RNA, 219 nt, synthetic construct
  source 1..219: mol_type = other RNA
gggtgatatacgaccagttttccaaacccctatcacgtaagcattaagtatctactggtctatatcatcgatgaatgataagaaatgactatccctaacccctatcacgtaagcattaaatgtaaagtcattcttatcagcacctcccattagtatgttaagaaattaaacccctatcacgtaagcattaaccttaacttaacaactaatgtacaacca

SEQ ID NO: 578 - RNA, 231 nt, synthetic construct
  source 1..231: mol_type = other RNA
gggtgatatacgaccagtaaacttataacccctatcacgtaagcattaaaatccaaaactggtctatatcatacccatgtgataagaaatgactacctatacaacccctatcacgtaagcattaaaccagcacagtcattcttatcaatcattaccattagtatgttaaggcaacataaacccctatcacgtaagcattaaaatctcaacttaacaactaatgactcatcc

SEQ ID NO: 579 - RNA, 255 nt, synthetic construct
  source 1..255: mol_type = other RNA
gggtcaatacagattatgttcctcaaatctaacccctatcacgtaagcattaagtccttaaagtccataatcgtattgacaacttccctatatacttcaaggccctaacttcgcaacccctatcacgtaagcattaatactccacacccccttgaatatatagtcataaatctgtttattgacatgactcttgttctcaacccctatcacgtaagcattaattccatctttcccatgtcataaacagcctccatc

SEQ ID NO: 580 - RNA, 411 nt, synthetic construct
  source 1..411: mol_type = other RNA
gggtgatatacgaccagtgacgaacccctatcacgtaagcattaaataaactggtctatatcacaagactatgataagaaatgactgaagaacccctatcacgtaagcattaacctaagtcattcttatcagctatttccattagtatgttaagagaaaacccctatcacgtaagcattaaatgacttaacaactaatgaattacaatcaatacagattatgactcaacccctatcacgtaagcattaaaacacataatcgtattgacataccttctatatacttcaagggaaaaacccctatcacgtaagcattaatagaccttgaatatatagccgatttcctgtttattgacatgacccaacccctatcacgtaagcattaacctccatgtcataaacagttataccg

SEQ ID NO: 581 - RNA, 435 nt, synthetic construct
  source 1..435: mol_type = other RNA
gggtgatatacgaccagtaatttcaacccctatcacgtaagcattaaacacccactggtctatatcaaactttaatgataagaaatgactactccgaacccctatcacgtaagcattaatattcaagtcattcttatcaaactcatccattagtatgttaagtcgtctaacccctatcacgtaagcattaacaaatccttaacaactaatgttcatgcctcaatacagattatggagcccaacccctatcacgtaagcattaaaaagagcataatcgtattgatttcgaacctatatacttcaaggctatacaacccctatcacgtaagcattaagaactaccttgaatatatagatcaattactgtttattgacatgaccagcaacccctatcacgtaagcattaacgactccatgtcataaacagactaacca

SEQ ID NO: 582 - RNA, 459 nt, synthetic construct
  source 1..459: mol_type = other RNA
gggtgatatacgaccagtaaacttataacccctatcacgtaagcattaaaatccaaaactggtctatatcatacccatgtgataagaaatgactacctatacaacccctatcacgtaagcattaaaccagcacagtcattcttatcaatcattaccattagtatgttaaggcaacataaacccctatcacgtaagcattaaaatctcaacttaacaactaatgactcatcctcaatacagattatgactgaccgaacccctatcacgtaagcattaactatattccataatcgtattgaacttcacactatatacttcaaggaacctactaacccctatcacgtaagcattaactgaactcccttgaatatatagccgttcacctgtttattgacatggtcagtccaacccctatcacgtaagcattaatatcgttacatgtcataaacagttagttag

SEQ ID NO: 583 - RNA, 507 nt, synthetic construct
  source 1..507: mol_type = other RNA
gggtgatatacgaccagtcactccttaacaaacccctatcacgtaagcattaatatacctgccctactggtctatatcagtacctgctgataagaaatgacttccacaaacagcaacccctatcacgtaagcattaaacctcacatctcagtcattcttatcacctttcctcattagtatgttaagatagccatcaccaacccctatcacgtaagcattaagagaatcataaacttaacaactaatgctgtcatatcaatacagattatgttcctcaaatctaacccctatcacgtaagcattaagtccttaaagtccataatcgtattgacaacttccctatatacttcaaggccctaacttcgcaacccctatcacgtaagcattaatactccacacccccttgaatatatagtcataaatctgtttattgacatgactcttgttctcaacccctatcacgtaagcattaattccatctttcccatgtcataaacagcctccatc

SEQ ID NO: 584 - RNA, 513 nt, synthetic construct; identical to SEQ ID NO: 302
  source 1..513: mol_type = other RNA

SEQ ID NO: 585 - RNA, 495 nt, synthetic construct
  source 1..495: mol_type = other RNA
gggtgatatacgaccagtttattctccgacgaccatggctcagactgttaagactcctacccactggtcgtatatcacccgtctttgataagaaatgactcccttccttatcgaccatggctcagactgttactatacctttccagtcatttcttatcacaactttacattagtatgttaagtgcacctaattcgaccatggctcagactgttaacactacctattcttaacatactaatgatttacaatcaatacagattatgttctcattatacgaccatggctcagactgttacaaatctcatctcataatctgtattgaattctgtcctatatacttcaaggatctatcacgacgaccatggctcagactgttacatacataactaccttgaagtatatagaaactcctctgtttattgacatgtctaaacgcaccgaccatggctcagactgttactattcctctcccatgtcaataaacagtcaccaaa

SEQ ID NO: 586 - RNA, 501 nt, synthetic construct; identical to SEQ ID NO: 306
  source 1..501: mol_type = other RNA

SEQ ID NO: 587 - RNA, 10 nt, synthetic construct
  source 1..10: mol_type = other RNA
gmwgvwgrag

SEQ ID NO: 588 - DNA, 51 bp, synthetic construct
  source 1..51: mol_type = other DNA
tctgcctccccagttcaagtggttctcctgcctcagcctcccgagtagctg

SEQ ID NO: 589 - DNA, 51 bp, synthetic construct
  source 1..51: mol_type = other DNA
cccacctcccgggttcaagtgatcctcctgcctcagcctcccgagtagctg

SEQ ID NO: 590 - DNA, 44 bp, synthetic construct
  source 1..44: mol_type = other DNA
tccgcctcccgggttcaagtgattctcctacctcagcctcccga

SEQ ID NO: 591 - DNA, 51 bp, synthetic construct
  source 1..51: mol_type = other DNA
tccgcctcccaggttcaagcgattctcctgcctcagcctcccgagtagctg

SEQ ID NO: 592 - DNA, 51 bp, synthetic construct
  source 1..51: mol_type = other DNA
tccgcctcccgggttcaagcgattctcctgcctcagcctcccgagtagctg

SEQ ID NO: 593 - DNA, 42 bp, synthetic construct
  source 1..42: mol_type = other DNA
cgcctcccgggttcaagcgattctcctgcctcagcctcccga

SEQ ID NO: 594 - DNA, 84 bp, synthetic construct
  source 1..84: mol_type = other DNA
tgatatacgaccagttcagactacacaacccctatcacgtaagcgattaagaaactcaatatactggtctatatcattatgagc

SEQ ID NO: 595 - RNA, 23 nt, synthetic construct
  source 1..23: mol_type = other RNA
caaagtgcttacagtgcaggtag

SEQ ID NO: 596 - DNA, 24 bp, synthetic construct
  source 1..24: mol_type = other DNA
ctacctgcactgtaagcanctttg

SEQ ID NO: 597 - DNA, 23 bp, synthetic construct
  source 1..23: mol_type = other DNA
ctacctgcactgatgcanctttg